(19) United States
(12) Patent Application Publication
(10) Pub. No.: US 2022/0380378 A1
(43) Pub. Date: Dec. 1, 2022

(54) ANDROGEN RECEPTOR MODULATORS AND METHODS FOR THEIR USE

(71) Applicant: ESSA Pharma, Inc., Vancouver (CA)

(72) Inventors: Han-Jie ZHOU, Foster City, CA (US); Peter Virsik, Portola Valley, CA (US); Berenger Biannic, San Carlos, CA (US)

(21) Appl. No.: 17/727,054

(22) Filed: Apr. 22, 2022

Related U.S. Application Data
(60) Provisional application No. 63/250,538, filed on Sep. 30, 2021.
(60) Provisional application No. 63/178,304, filed on Apr. 22, 2021.

(51) Int. Cl. C07D 487/10; C07D 239/47; C07D 403/12; C07D 405/12; C07D 239/42; C07D 403/04; C07D 471/04; C07D 413/12; C07D 405/14; C07D 471/10

(52) U.S. Cl. CPC C07D 487/10; C07D 239/47; C07D 403/12; C07D 405/12; C07D 239/42; C07D 403/04; C07D 471/04; C07D 413/12; C07D 405/14; C07D 471/10

(57) ABSTRACT

The present invention relates to compounds of formula (A), (A-I), (A-II), (A-III), (B), (B-I), (C), (D), (E), (E-I), (F), (G), (H), (J), and (K), or any subgenera thereof, or a pharmaceutically acceptable salt, tautomer or stereoisomer. The compounds of the present disclosure are useful in modulating androgen receptor activity and for treating cancer, including prostate cancer.

### CROSS-REFERENCE TO RELATED APPLICATIONS

This application claims the benefit of and priority to U.S. Provisional Application No. 63/178,304 filed Apr. 22, 2021 and U.S. Provisional Application No. 63/250,538 filed Sep. 30, 2021, each disclosure is hereby incorporated by reference in its entirety.

### FIELD OF THE INVENTION

The present disclosure generally relates to tricyclic compounds and their use for treatment of various indications. In particular the disclosure relates to tricyclic compounds and their use for treatment of various cancers, for example prostate cancer, including but not limited to, primary/localized prostate cancer (newly diagnosed), locally advanced prostate cancer, recurrent prostate cancer, non-metastatic castration-resistant prostate cancer, metastatic prostate cancer, metastatic castration-resistant prostate cancer (CRPC), and hormone-sensitive prostate cancer. This invention also relates to tricyclic compounds and their use for modulating androgen receptor (AR) activity, including truncated AR.

### BACKGROUND OF THE INVENTION

Androgens mediate their effects through the androgen receptor (AR). Androgens play a role in a wide range of developmental and physiological responses and are involved in male sexual differentiation, maintenance of spermatogenesis, and male gonadotropin regulation (R. K. Ross, G. A. Coetzee, C. L. Pearce, J. K. Reichardt, P. Bretsky, L. N. Kolonel, B. E. Henderson, E. Lander, D. Altshuler & G. Daley, Eur Urol 35, 355-361 (1999); A. A. Thomson, Reproduction 121, 187-195 (2001); N. Tanji, K. Aoki & M. Yokoyama, Arch Androl 47, 1-7 (2001)). Several lines of evidence show that androgens are associated with the development of prostate carcinogenesis. Firstly, androgens induce prostatic carcinogenesis in rodent models (R. L. Noble, Cancer Res 37, 1929-1933 (1977); R. L. Noble, Oncology 34, 138-141 (1977)) and men receiving androgens in the form of anabolic steroids have a higher incidence of prostate cancer (J. T. Roberts & D. M. Essenhigh, Lancet 2, 742 (1986); J. A. Jackson, J. Waxman & A. M. Spiekerman, Arch Intern Med 149, 2365-2366 (1989); P. D. Guinan, W. Sadoughi, H. Alsheik, R. J. Ablin, D. Alrenga & I. M. Bush, Am J Surg 131, 599-600 (1976)). Secondly, prostate cancer does not develop if humans or dogs are castrated before puberty (J. D. Wilson & C. Roehrborn, J Clin Endocrinol Metab 84, 4324-4331 (1999); G. Wilding, Cancer Surv 14, 113-130 (1992)). Castration of adult males causes involution of the prostate and apoptosis of prostatic epithelium while eliciting no effect on other male external genitalia (E. M. Bruckheimer & N. Kyprianou, Cell Tissue Res 301, 153-162 (2000); J. T. Isaacs, Prostate 5, 545-557 (1984)). This dependency on androgens provides the underlying rationale for treating prostate cancer with chemical or surgical castration (androgen ablation), also known as androgen ablation therapy (ABT) or androgen depravation therapy (ADT).

Androgens also play a role in female diseases such as polycystic ovary syndrome as well as cancers. One example is ovarian cancer where elevated levels of androgens are associated with an increased risk of developing ovarian cancer (K. J. Helzlsouer, A. J. Alberg, G. B. Gordon, C. Longcope, T. L. Bush, S. C. Hoffman & G. W. Comstock, JAMA 274, 1926-1930 (1995); R. J. Edmondson, J. M. Monaghan & B. R. Davies, Br J Cancer 86, 879-885 (2002)). The AR has been detected in a majority of ovarian cancers (H. A. Risch, J Natl Cancer Inst 90, 1774-1786 (1998); B. R. Rao & B. J. Slotman, Endocr Rev 12, 14-26 (1991); G. M. Clinton & W. Hua, Crit Rev Oncol Hematol 25, 1-9 (1997)), whereas estrogen receptor-alpha (ERa) and the progesterone receptor are detected in less than 50% of ovarian tumors.

The only effective treatment available for advanced prostate cancer is the withdrawal of androgens which are essential for the survival of prostate luminal cells. Androgen ablation therapy causes a temporary reduction in tumor burden concomitant with a decrease in serum prostate-specific antigen (PSA). Unfortunately, prostate cancer can eventually grow again in the absence of testicular androgens (castration-resistant disease) (Huber et al 1987 Scand J. Urol Nephrol. 104, 33-39). Castration-resistant prostate cancer that is still driven by AR is biochemically characterized before the onset of symptoms by a rising titre of serum PSA (Miller et al 1992 J. Urol. 147, 956-961). Once the disease becomes castration-resistant most patients succumb to their disease within two years.

The AR has distinct functional domains that include the carboxy-terminal ligand-binding domain (LBD), a DNA-binding domain (DBD) comprising two zinc finger motifs, and an N-terminus domain (NTD) that contains two transcriptional activation units (tau1 and tau5) within activation function-1 (AF-1). Binding of androgen (ligand) to the LBD of the AR results in its activation such that the receptor can effectively bind to its specific DNA consensus site, termed the androgen response element (ARE), on the promoter and enhancer regions of “normally” androgen regulated genes, such as PSA, to initiate transcription. The AR can be activated in the absence of androgen by stimulation of the cAMP-dependent protein kinase (PKA) pathway, with interleukin-6 (IL-6) and by various growth factors (Culig et al 1994 Cancer Res. 54, 5474-5478; Nazareth et al 1996 J. Biol. Chem. 271, 19900-19907; Sadar 1999 J. Biol. Chem. 274, 7777-7783; Ueda et al 2002 A J. Biol. Chem. 277, 7076-7085; and Ueda et al 2002 B J. Biol. Chem. 277, 38087-38094). The mechanism of ligand-independent transformation of the AR has been shown to involve: 1) increased nuclear AR protein suggesting nuclear translocation; 2) increased AR/ARE complex formation; and 3) the AR-NTD (Sadar 1999 J. Biol. Chem. 274, 7777-7783; Ueda et al 2002 A J Biol. Chem. 277, 7076-7085; and Ueda et al 2002 B J. Biol. Chem. 277, 38087-38094). The AR can be activated in the absence of testicular androgens by alternative signal transduction pathways in castration-resistant disease, which is consistent with the finding that nuclear AR protein is present in secondary prostate cancer tumors (Kim et al 2002 Am. J. Pathol. 160, 219-226; and van der Kwast et al 1991 Inter. J. Cancer 48, 189-193).

The AR-NTD is also a target for drug development (e.g. WO 2000/001813; Myung et al. J. Clin. Invest 2013, 123, 2948), since the NTD contains Activation-Function-1 (AF-1) which is the essential region required for AR transcriptional activity (Jenster et al 1991. Mol Endocrinol. 5, 1396-404). The AR-NTD importantly plays a role in activation of the AR in the absence of androgens (Sadar, M. D. 1999 J. Biol. Chem. 274, 7777-7783; Sadar M D et al 1999 Endocr Relat Cancer. 6, 487-502; Ueda et al 2002 J. Biol. Chem. 277, 7076-7085; Ueda 2002 J. Biol. Chem. 277, 38087-38094; Blaszczyk et al 2004 Clin Cancer Res. 10, 1860-9; Dehm et al 2006 J Biol Chem. 28, 27882-93; Gregory et al 2004 J Biol Chem. 279, 7119-30). The AR-NTD is important in hormonal progression of prostate cancer as shown by application of decoy molecules (Quayle et al 2007, Proc Natl Acad Sci USA. 104, 1331-1336).

While the crystal structure has been resolved for the AR C-terminus LBD, this has not been the case for the NTD due to its high flexibility and intrinsic disorder in solution (Reid et al 2002 J. Biol. Chem. 277, 20079-20086) thereby hampering virtual docking drug discovery approaches. Compounds that modulate AR, potentially through interaction with NTD domain, include the bisphenol compounds disclosed in published PCT Nos: WO 2010/000066, WO 2011/082487; WO 2011/082488; WO 2012/145330; WO 2012/139039; WO 2012/145328; WO 2013/028572; WO 2013/028791; WO 2014/179867; WO 2015/031984; WO 2016/058080; WO 2016/058082; WO 2016/112455; WO 2016/141458; WO 2017/177307; WO 2017/210771; WO 2018/045450, and WO 2019/226991, and WO 2020/081999, and which are hereby incorporated by reference in their entireties.

While significant advances have been made in this field, there remains a need for improved treatment for AR-mediated disorders including prostate cancer, especially metastatic castration-resistant prostate cancer. Development of compounds, via unique interactions with AR NTD, would provide patients alternative options and new hope.

### SUMMARY OF THE INVENTION

The compounds of the present disclosure are androgen receptor modulators which may be useful in treating various diseases and conditions as disclosed herein.

In one embodiment, the present disclosure provides compounds comprising the structure of formula (I-A):

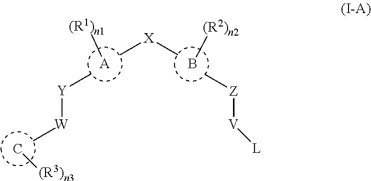

or a pharmaceutically acceptable salt, tautomer, stereoisomer or prodrug thereof, wherein:
A is a 4- to 15-membered ring selected from aryl, carbocyclyl, heteroaryl, or heterocyclyl;
B is a 4- to 15-membered ring selected from aryl, carbocyclyl, heteroaryl, or heterocyclyl;
C is a 3- to 10-membered aryl, carbocyclyl, heteroaryl, or heterocyclyl;
X is a bond, —(CR⁵R⁶)ₜ—, —C(═O)—, or —NR⁷;
Y is a bond, —(CR⁸R⁹)ₘ—, —O—, —S—, —S(═O)—, —SO₂—, —NR⁷—, or —N(COCH₃)—;
W is a bond, —(CR^{8a}R^{9a})ₘ—, —C(═O)—, —NR⁷—, —N(R⁷)CO—, —CONR⁷—, or —NSO₂R⁷—;
Z is a bond, —CH₂—, —C(CH₃)H—, —O—, —S—, —NH—, —NCH₃—, or —N(COCH₃)—;
V is a bond, —(CR^{8a}R^{9a})ₘ—, —CR^{8a}═CR^{9a}—, or phenylene;
L is hydrogen, halogen, —CF₂R¹⁰, —CF₃, —CN, —OR¹⁰, —SO₂R¹⁷, —NR¹¹R¹², or —CONR¹¹R¹²;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, oxo, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —NR¹⁴COOR¹⁶, —(C₁-C₆ alkyl)-NR¹⁴COR¹⁶, —CONR¹⁴R¹⁵, —(C₁-C₃ alkyl)-CONR¹⁴R⁵, —SO₂NR¹⁴R¹⁵, —(C₁-C₃ alkyl)-SO₂NR¹⁴R⁵, —SO₂(C₁-C₃ alkyl), —(C₁-C₆ alkyl)-SO₂(C₁-C₃ alkyl), optionally substituted carbocyclyl, optionally substituted heterocyclyl, optionally substituted aryl, or optionally substituted heteroaryl;
R⁵ and R⁶ are each independently hydrogen, halogen, —OH, optionally substituted C₁-C₃ alkyl, optionally substituted C₂-C₃ alkenyl, optionally substituted C₂-C₃ alkynyl, or C₁-C₃ alkoxy; or R⁵ and R⁶ taken together form an optionally substituted 3- to 6-membered carbocyclyl or heterocyclyl;
R⁷ is hydrogen, C₁-C₆ alkyl, C₂-C₆ alkenyl, or C₂-C₆ alkynyl;
R⁸ and R⁹ are each independently hydrogen, halogen, or C₁-C₃ alkyl;
R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴—(C₁-C₃ alkyl)-NR¹³R¹⁴, —NR¹⁴COR¹⁶—(C₁-C₃ alkyl)-NR¹⁴COR¹⁶, —CONR¹⁴R¹⁵, or —(C₁-C₃ alkyl)-CONR¹⁴R¹⁵; or R^{8a} and R^{9a} taken together form an optionally substituted 3- to 6-membered carbocyclyl or heterocyclyl;
R¹⁰ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl;
R¹¹ and R¹² are each independently hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl; or R¹¹ and R¹² taken together form an optionally substituted heterocyclyl which optionally contains one or two additional heteroatoms selected from N, S, or O;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form an optionally substituted 3- to 6-membered heterocyclyl;
R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;
R¹⁷ is hydrogen, optionally substituted C₁-C₃ alkyl, optionally substituted C₂-C₃ alkenyl, or optionally substituted C₂-C₃ alkynyl;
each m is independently 0, 1, or 2;
n1 and n2 are each independently 0, 1, or 2;
n3 is 0, 1, 2, 3, 4 or 5; and
t is 0, 1 or 2.

In one embodiment, the present disclosure provides compounds comprising the structure of formula (I-B):

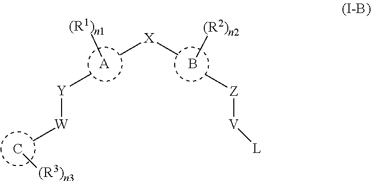

or a pharmaceutically acceptable salt, tautomer, stereoisomer or prodrug thereof, wherein:
A is a 4- to 15-membered ring selected from aryl, carbocyclyl, heteroaryl, or heterocyclyl;
B is a 4- to 15-membered ring selected from aryl, carbocyclyl, heteroaryl, or heterocyclyl;
C is a 3- to 10-membered aryl, carbocyclyl, heteroaryl, or heterocyclyl;
X is a bond, —(CR⁵R⁶)ₜ—, —C(═O)—, or —NR⁷;
Y is a bond, —(CR⁸R⁹)ₘ—, —O—, —S—, —S(═O)—, —SO₂—, —NR⁷—, or —N(COCH₃)—;
W is a bond, —(CR^{8a}R^{9a})ₘ—, —C(═O)—, —N(R⁷)CO—, —CONR⁷—, or —NSO₂R⁷—;
Z is a bond, —CH₂—, —C(CH₃)H—, —O—, —S—, —NH—, —NCH₃—, or —N(COCH₃)—;
V is a bond, —(CR^{8a}R^{9a})ₘ—, or phenylene;
L is hydrogen, halogen, —CF₂R¹⁰, —CF₃, —CN, —OR¹⁰; —NR¹¹R¹², or —CONR¹¹R¹²;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, oxo, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —NR¹⁴COOR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), —(C₁-C₆ alkyl)-SO₂(C₁-C₃ alkyl), optionally substituted carbocyclyl, optionally substituted heterocyclyl, optionally substituted aryl, or optionally substituted heteroaryl;
R⁵ and R⁶ are each independently hydrogen, halogen, —OH, optionally substituted C₁-C₃ alkyl, optionally substituted C₂-C₃ alkenyl, optionally substituted C₂-C₃ alkynyl, or C₁-C₃ alkoxy; or R⁵ and R⁶ taken together form an optionally substituted 3- to 6-membered carbocyclyl or heterocyclyl;
R⁷ is hydrogen, C₁-C₆ alkyl, C₂-C₆ alkenyl, or C₂-C₆ alkynyl;
R⁸ and R⁹ are each independently hydrogen, halogen, or C₁-C₃ alkyl;
R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{9a} taken together form an optionally substituted 3- to 6-membered carbocyclyl or heterocyclyl;
R¹⁰ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl;
R¹¹ and R¹² are each independently hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl; or R¹¹ and R¹² taken together form an optionally substituted heterocyclyl which optionally contains one or two additional heteroatoms selected from N, S, or O;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form an optionally substituted 3- to 6-membered heterocyclyl;
R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;
each m is independently 0, 1, or 2;
n1 and n2 are each independently 0, 1, or 2;
n3 is 0, 1, 2, 3, 4 or 5; and
t is 0, 1 or 2.

In one embodiment, the present disclosure provides compounds of formula (II):

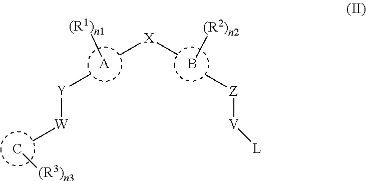

or a pharmaceutically acceptable salt, tautomer, stereoisomer or prodrug thereof, wherein:
A is a 4- to 15-membered ring selected from aryl, carbocyclyl, heteroaryl, or heterocyclyl;
B is a 4- to 15-membered ring selected from aryl, carbocyclyl, heteroaryl, or heterocyclyl;
C is a heteroaryl ring;
X is a bond, —(CR⁵R⁶)ₜ—, —C(═O)—, or —NR⁷;
Y is a bond, —(CR⁸R⁹)ₘ—, —O—, —S—, —S(═O)—, —SO₂—, —NR⁷—, or —N(COCH₃)—;
W is a bond, —(CR^{8a}R^{9a})ₘ—, —C(═O)—, —N(R⁷)CO—, —CONR⁷—, or —NSO₂R⁷—;
Z is a bond, —CH₂—, —C(CH₃)H—, —O—, —S—, —NH—, —NCH₃—, or —N(COCH₃)—;
V is —CH₂— or phenylene and L is halogen, —NH₂, —CHCl₂, —CCl₃, or —CF₃; or
V is —CH₂CH₂— and L is halogen or —NH₂;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, oxo, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;

R³ is selected from halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —NR¹⁴COOR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is hydrogen, C₁-C₆ alkyl, C₂-C₆ alkenyl, or C₂-C₆ alkynyl;
R⁸ and R⁹ are each independently hydrogen, halogen, or C₁-C₃ alkyl;
R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{9a} taken together form an optionally substituted 3- to 6-membered carbocyclyl or heterocyclyl;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form an optionally substituted 3- to 6-membered heterocyclyl;
R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;
each m is independently 0, 1, or 2;
n1 and n2 are each independently 0, 1, or 2;
n3 is 1, 2, 3, 4 or 5; and
t is 0, 1 or 2.

In one embodiment, the present disclosure provides compounds of formula (A-I):

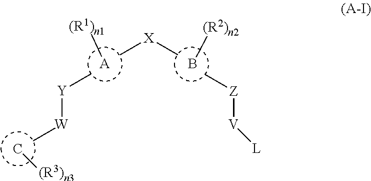

or a pharmaceutically acceptable salt, tautomer, stereoisomer or prodrug thereof, wherein:
A is a fused or a bridged bicyclic 5- to 15-membered ring selected from aryl, carbocyclyl, heteroaryl, or heterocyclyl;
B is a phenyl;
C is a heteroaryl ring;
X is a bond, —CH₂—, —C(CH₃)₂—, or —C(═O)—;
Y is a bond or —O—;
W is —CH₂— or —C(CH₃)H—;
Z is a bond or —O—;
V is —CH₂— and L is halogen, —CHCl₂, —CCl₃, or —CF₃; or
V is —CH₂CH₂— and L is halogen;
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —NHCOO(C₁-C₃ alkyl), —N(CH₃)CO(C₁-C₃ alkyl), or —N(CH₃)COO(C₁-C₃ alkyl);
n1 and n2 are each independently 0, 1, or 2; and
n3 is 1, 2, 3, 4 or 5.

In one embodiment, the present disclosure provides compounds of formula (A-II):

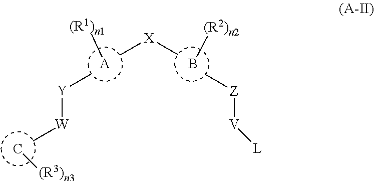

or a pharmaceutically acceptable salt, tautomer, stereoisomer or prodrug thereof, wherein:
A is a fused or a bridged bicyclic 5- to 15-membered ring selected from aryl, carbocyclyl, heteroaryl, or heterocyclyl;
B is a phenyl or a 6-membered heteroaryl or heterocyclyl;
C is a 3- to 10-membered aryl, carbocyclyl, heteroaryl, or heterocyclyl;
X is a bond, —CH₂—, —C(CH₃)₂—, or —C(═O)—;
Y is a bond, —CH₂—, —NH—, or —O—;
W is a bond, —CH₂—, —CH₂CH₂—, —C(CH₃)H—, —N(R⁷)CO—, or —CONR⁷—;
Z is a bond or —O—;
V is a bond, —CH₂(CR^{8a′}R^{9a′})—, or —(CR^{8a}R^{9a})ₘ—;
L is hydrogen, halogen, —CF₂R¹⁰, —CF₃, —CN, —OR¹⁰, —NR¹¹R¹², or —CONR¹¹R¹²;
R¹ and R² are each independently hydrogen, halogen, oxo, —CN, or —CF₃;
at least one R³ is —CH₂NHSO₂CH₃, —CH₂N(CH₃)SO₂CH₃, —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), or —N(CH₃)COO(C₁-C₃ alkyl);
R⁷ is hydrogen or C₁-C₃ alkyl;
R^{8a} and R^{9a} are each independently hydrogen, halogen, or C₁-C₃ alkyl;
R^{8a′} and R^{9a′} taken together form an optionally substituted 3- to 6-membered carbocyclyl or heterocyclyl containing one, two, or three heteroatoms selected from N, S, or O;
R¹⁰ is hydrogen, or C₁-C₃ alkyl;
R¹¹ and R¹² are each independently hydrogen or C₁-C₃ alkyl; or R¹¹ and R¹² taken together form an optionally substituted heterocyclyl which optionally contains one or two additional heteroatoms selected from N, S, or O;
m is 0, 1, or 2;
n1 and n2 are each independently 0, 1, or 2; and
n3 is 1, 2, 3, 4 or 5.

In one embodiment, the present disclosure provides compounds of formula (A-III):

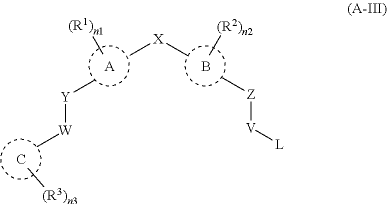

or a pharmaceutically acceptable salt, tautomer, stereoisomer or prodrug thereof, wherein:
A is a fused or a bridged bicyclic 5- to 15-membered ring selected from aryl, carbocyclyl, heteroaryl, or heterocyclyl;
B is a phenyl or a 6-membered heteroaryl or heterocyclyl;
C is a 4- to 10-membered aryl, carbocyclyl, heteroaryl, or heterocyclyl;
X is a bond, —CH₂—, —C(CH₃)₂—, or —C(═O)—;
Y is a bond, —CH₂—, —C(CH₃)H—, —NH—, or —O—;
W is —CH₂—, —CH₂CH₂—, —C(CH₃)H—, —NR⁷—, —N(R⁷)CO—, or —CONR⁷—;
Z is a bond or —O—;
V is a bond, —CH₂(CR^{8a′}R^{9a′})—, —CR^{8a}═CR^{9a}—, or —(CR^{8a}R^{9a})ₘ—;
L is hydrogen, halogen, —CF₂R¹⁰, —CF₃, —CN, —OR¹⁰, —NR¹¹R¹², —SO₂R¹⁷, or —CONR¹¹R¹²;
R¹ and R² are each independently hydrogen, halogen, oxo, —CN, or —CF₃;
at least one R³ is —CH₂NHSO₂CH₃, —CH₂N(CH₃)SO₂CH₃, —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), or —N(CH₃)COO(C₁-C₃ alkyl);
R⁷ is hydrogen or C₁-C₃ alkyl;
R^{8a} and R^{9a} are each independently hydrogen, halogen, or C₁-C₃ alkyl;
R^{8a′} and R^{9a′} taken together form an optionally substituted 3- to 6-membered carbocyclyl or heterocyclyl containing one, two, or three heteroatoms selected from N, S, or O;
R¹⁰ is hydrogen, or C₁-C₃ alkyl;
R¹¹ and R¹² are each independently hydrogen or C₁-C₃ alkyl; or R¹¹ and R¹² taken together form an optionally substituted heterocyclyl which optionally contains one or two additional heteroatoms selected from N, S, or O;
R¹⁷ is hydrogen or C₁-C₃ alkyl;
m is 0, 1, or 2;
n1 and n2 are each independently 0, 1, or 2; and
n3 is 1, 2, 3, 4 or 5.

In one embodiment of the compounds of formula (A), (A-I), (A-II), and/or (A-III), A is a 6,6-fused ring, 6,5-fused ring, or 5,6-fused ring, each optionally substituted with one or two R¹.

In one embodiment of the compounds of formula (A), (A-I), (A-II), and/or (A-III), A is

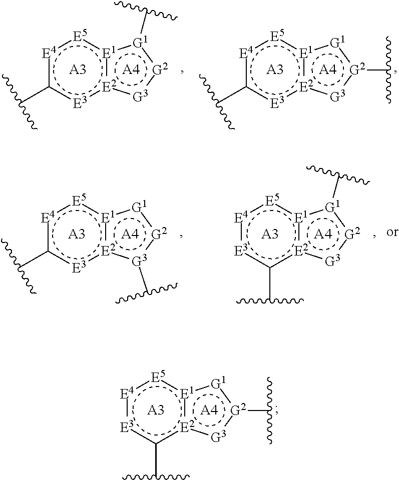

wherein:
ring A3 is aromatic;
E¹, E², E³, E⁴, and E⁵ are each independently, C, CR¹ or N;
G¹, G², and G³, are each independently, C, CR¹, C(R¹)₂, O, S, N, or NR¹;
wherein at least two of E¹, E², E³, E⁴, and E⁵ is C or CR¹; and
wherein at least two of E¹, E², G¹, G², and G³, is C, CR¹ or C(R¹)₂.

In one embodiment of the compounds of formula (A), (A-I), (A-II), and/or (A-III), A is

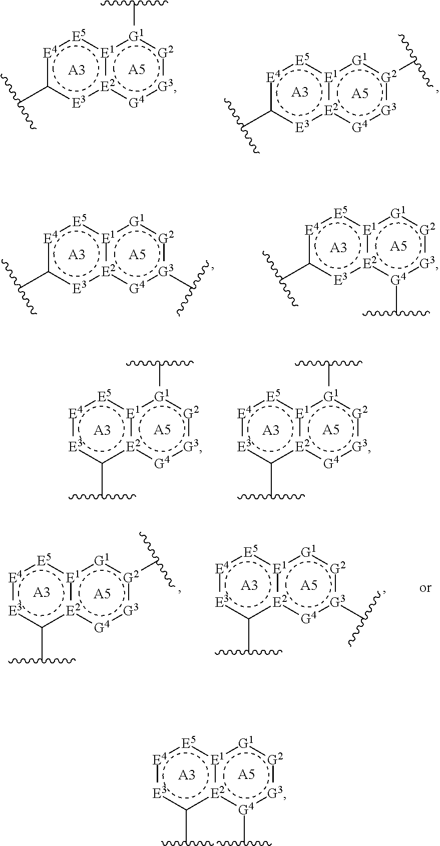

wherein:
ring A3 is aromatic;
E¹, E², E³, E⁴, and E⁵ are each independently, C, CR¹ or N;
G¹, G², G³, and G⁴, are each independently, C, CR¹, C(R¹)₂, O, S, N, or NR¹;
wherein at least two of E¹, E², E³, E⁴, and E⁵ is C or CR¹; and
wherein at least three of E¹, E², G¹, G², G³, and G⁴, is C, CR¹ or C(R¹)₂.

In one embodiment of the compounds of formula (A), (A-I), (A-II), and/or (A-III), A is a ring selected from bicyclo[1.1.1]pentane, 4,5,6,7-tetrahydroindole, indoline, indole, pyrazolo[1,5-a]pyridine, imidazo[1,2-a]pyridine, indazole, benzo[d]imidazole, benzo[d]isoxazole, benzo[b]thiophene, 1,3-dihydroisobenzofuran, quinazoline, 3,4-dihydrobenzo[b][1,4]oxazine, benzo[d][1,2,3]triazole, naphthalene, 1,2-dihydronaphthalene, 1,2,3,4-tetrahydronaphthalene, 2,3-dihydroindene, 2,3-dihydrobenzo[b][1,4]dioxine, isoindoline, or isoindolin-1-one, each ring is optionally substituted with one or two R¹. In one embodiment, A is

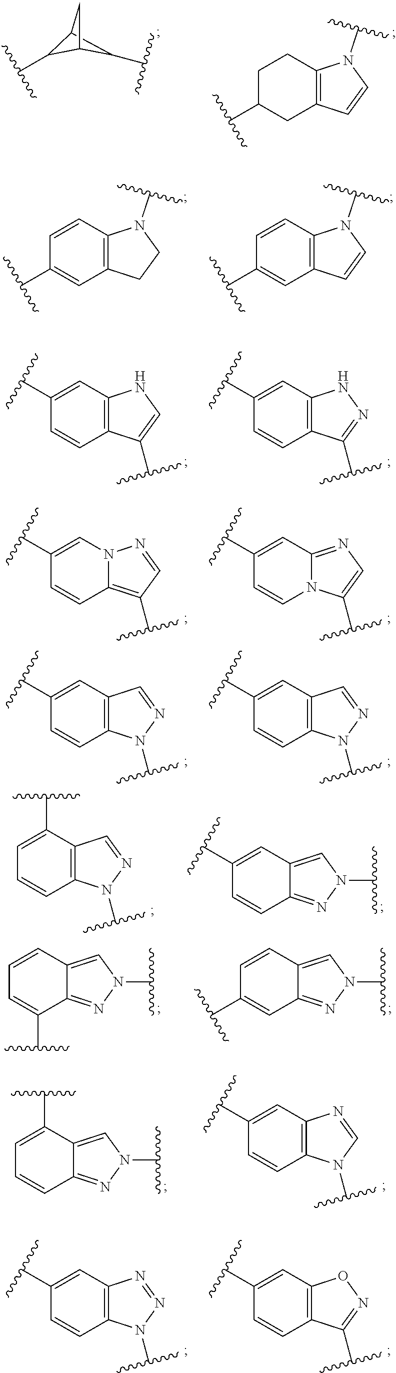

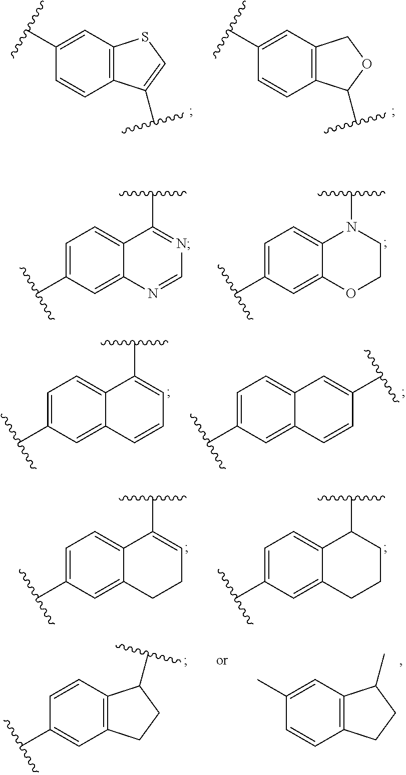

each ring is optionally substituted with one or two R¹.

In one embodiment of the compounds of formula (A), (A-I), (A-II), and/or (A-III), C is

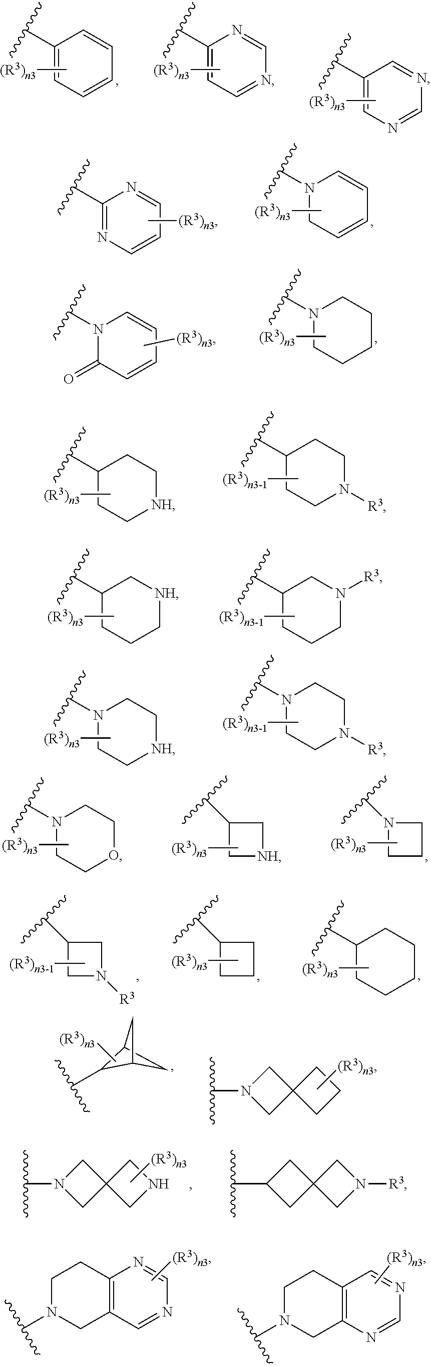

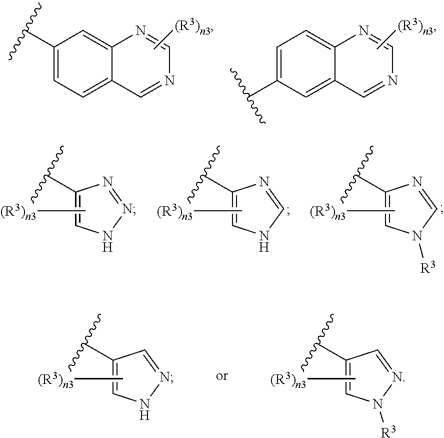

In one embodiment of the compounds of formula (A), (A-I), (A-II), and/or (A-III):
X is bond or —C(CH₃)₂—;
Y and Z are each —O—;
V is —CH₂— or —CH₂CH₂—;
L is halogen;
R¹ and R² are each independently hydrogen, halogen, —CN, or —CF₃; and
R¹⁶ is hydrogen, C₁-C₃ alkyl, C₂-C₃ alkenyl, or C₂-C₃ alkynyl.

In one embodiment of the compounds of formula (A), (A-I), (A-II), and/or (A-III):
W is —CH₂— or —C(CH₃)H—;
V is —CH₂CH₂—; and
R¹ and R² are each independently hydrogen, halogen, —CN, or —CF₃.

In one embodiment of the compounds of formula (A-III):
Y is —CH₂— or —C(CH₃)H—; and
W is —NH—.

In one embodiment of the compounds of formula (A-III), W is —NH—.

In one embodiment of the compounds of formula (A-III):
Y is —CH₂— or —C(CH₃)H—;
W is —NH—;
X is bond or —C(CH₃)₂—;
Z is —O—;
V is —CH₂— or —CH₂CH₂—;
L is halogen; and
R¹ and R² are each independently hydrogen, halogen, —CN, or —CF₃.

In one embodiment, the present disclosure provides compounds of formula (B):

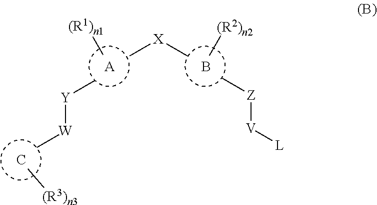

or a pharmaceutically acceptable salt, tautomer, stereoisomer or prodrug thereof, wherein:
A is a phenyl;
B is a 5- to 15-membered ring selected from aryl, carbocyclyl, heteroaryl, or heterocyclyl;
C is a heteroaryl ring;
X is a bond, —(CR⁵R⁶)ₜ—, or —C(═O)—;
Y is a bond, —(CR⁸R⁹)ₘ—, —O—, —S—, —S(═O)—, —SO₂—, —NR⁷—, or —N(COCH₃)—;
W is a bond, —(CR^{8a}R^{9a})ₘ—, —C(═O)—, —N(R⁷)CO—, —CONR⁷—, or —NSO₂R⁷—;
Z is a bond, —CH₂—, —C(CH₃)H—, —O—, —S—, —NH—, —NCH₃—, or —N(COCH₃)—;
V is a bond or —CH₂— and L is halogen, —NH₂, —CHCl₂, —CCl₃, or —CF₃; or
V is —CH₂CH₂— and L is halogen or —NH₂;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, oxo, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —NR¹⁴COOR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3-to 6-membered carbocyclyl or heterocyclyl;
R⁷ is hydrogen, C₁-C₆ alkyl, C₂-C₆ alkenyl, or C₂-C₆ alkynyl;
R⁸ and R⁹ are each independently hydrogen, halogen, or C₁-C₃ alkyl;
R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{9a} taken together form an optionally substituted 3- to 6-membered carbocyclyl or heterocyclyl;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form an optionally substituted 3- to 6-membered heterocyclyl;
R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;
each m is independently 0, 1, or 2;
n1 and n2 are each independently 0, 1, or 2;
n3 is 1, 2, 3, 4 or 5; and
t is 0, 1 or 2.

In one embodiment of the compounds of formula (B), B is a fused or a bridged bicyclic ring, optionally substituted with one or two R².

In one embodiment of the compounds of formula (B):
X is bond or —C(CH₃)₂—;
Y is —O—;
Z is a bond or —O—;
V is bond, —CH₂— or —CH₂CH₂—;
L is halogen;
R¹ and R² are each independently hydrogen, halogen, —CN, or —CF₃; and
R¹⁶ is hydrogen, C₁-C₃ alkyl, C₂-C₃ alkenyl, or C₂-C₃ alkynyl.

In one embodiment of the compounds of formula (B):
Y and Z are each —O—;
W is —CH₂— or —C(CH₃)H—;
V is —CH₂CH₂—; and
R¹ and R² are each independently hydrogen, halogen, —CN, or —CF₃.

In one embodiment, the present disclosure provides compounds of formula (B-I):

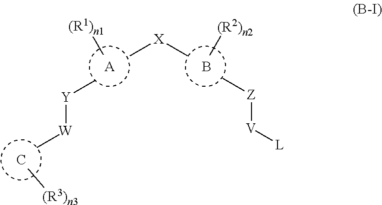

or a pharmaceutically acceptable salt, tautomer, stereoisomer or prodrug thereof, wherein:
A is a phenyl;
B is a fused or a bridged bicyclic 5- to 15-membered ring selected from aryl, carbocyclyl, heteroaryl, or heterocyclyl;
C is a heteroaryl ring;
X is a bond, —CH₂—, —C(CH₃)₂—, or —C(═O)—;
Y is a bond or —O—;
W is —CH₂— or —C(CH₃)H—;
Z is a bond or —O—;
V is a bond or —CH₂— and L is halogen, —CHCl₂, —CCl₃, or —CF₃; or
V is —CH₂CH₂— and L is halogen;
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —NHCOO(C₁-C₃ alkyl), —N(CH₃)CO(C₁-C₃ alkyl), or —N(CH₃)COO(C₁-C₃ alkyl);
n1 and n2 are each independently 0, 1, or 2; and
n3 is 1, 2, 3, 4 or 5.

In one embodiment of the compounds of formula (B) and/or (B-I), B is a 6,6-fused ring, 6,5-fused ring, or 5,6-fused ring, each optionally substituted with one or two R¹.

In one embodiment of the compounds of formula (B) and/or (B-I), B is

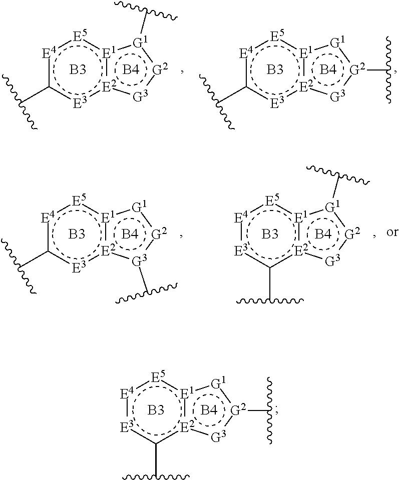

wherein:
ring B3 is aromatic;
E¹, E², E³, E⁴, and E⁵ are each independently, C, CR² or N;
G¹, G², and G³, are each independently, C, CR², C(R²)₂, O, S, N, or NR²;
wherein at least two of E¹, E², E³, E⁴, and E⁵ is C or CR²; and
wherein at least two of E¹, E², G¹, G², and G³, is C, CR² or C(R²)₂.

In one embodiment of the compounds of formula (I), (II), (B) or (B-I), B is

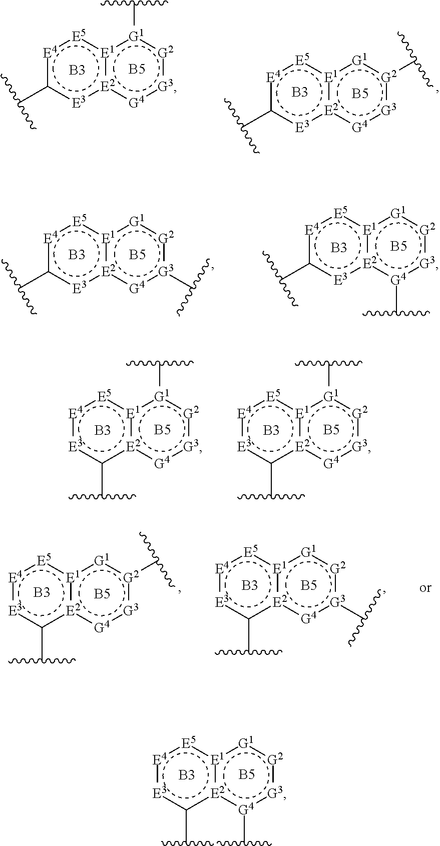

wherein:
ring B3 is aromatic;
E¹, E², E³, E⁴, and E⁵ are each independently, C, CR² or N;
G¹, G², G³, and G⁴, are each independently, C, CR², C(R²)₂, O, S, N, or NR²;
wherein at least two of E¹, E², E³, E⁴, and E⁵ is C or CR²; and
wherein at least three of E¹, E², G¹, G², G³, and G⁴, is C, CR² or C(R²)₂.

In one embodiment of the compounds of formula (B) and/or (B-I), B is ring selected from bicyclo[1.1.1]pentane, indoline, indole, indazole, quinazoline, 3,4-dihydro-2H-benzo[b][1,4]oxazine, 1H-benzo[d][1,2,3]triazole, naphthalene, 1,2-dihydronaphthalene, 1,2,3,4-tetrahydronaphthalene, 2,3-dihydro-1H-indene, 2,3-dihydrobenzo[b][1,4]dioxine, isoindoline, or isoindolin-1-one, each ring is optionally substituted with one or two R². In one embodiment, B is:

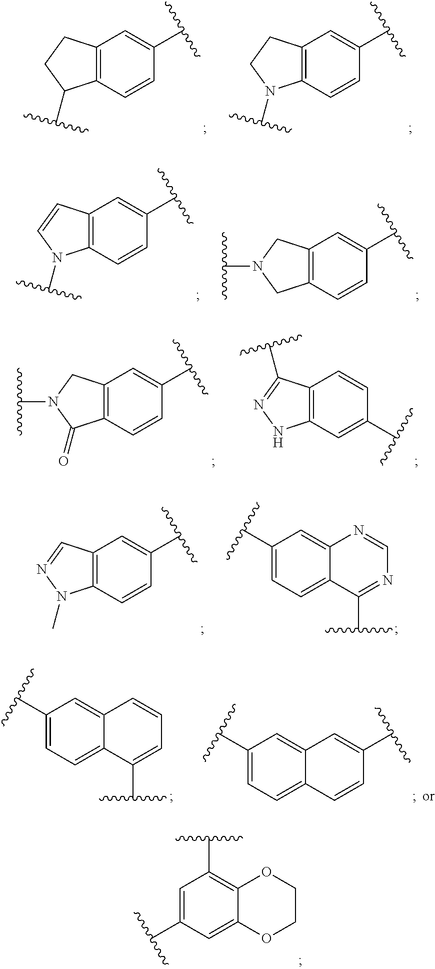

wherein each ring is optionally substituted with one or two R².

In one embodiment of the compounds of formula (B) and/or (B-I),
X is bond or —C(CH₃)₂—;
Y is —O—;
V is bond, —CH₂— or —CH₂CH₂—;
L is halogen;
R¹ and R² are each independently hydrogen, halogen, —CN, or —CF₃; and
R¹⁶ is hydrogen, C₁-C₃ alkyl, C₂-C₃ alkenyl, or C₂-C₃ alkynyl.

In one embodiment of the compounds of formula (B) and (B-I),
Y and Z are each —O—;
V is —CH₂CH₂—; and
R¹ and R² are each independently hydrogen, halogen, —CN, or —CF₃.

In one embodiment of the compounds of formula (II), (A), (A-I), (A-II), (A-III), (B), and/or (B-I), C is a pyrimidine ring.

In one embodiment of the compounds of formula (II), (A), (A-I), (A-II), (A-III), (B), and/or (B-I), R¹ and R² are each independently Cl, —CN, or —CF₃.

In one embodiment of the compounds of formula (II), (A), (A-I), (A-II), (A-III), (B), and/or (B-I), R³ is —NHSO₂CH₃, —NHSO₂CH₂CH₃, —SO₂NH₂, or —SO₂CH₃. In one embodiment, R³ is —NHSO₂CH₃, —NHSO₂CH₂CH₃, —SO₂NH₂, or —SO₂CH₃ and the other R³ is, if present (e.g., when n3 is 2, 3, or 5), —CN, C₁-C₃ alkyl, C₁-C₃ alkoxy, —SO₂(C₁-C₃ alkyl), —NH₂, —(C₁-C₃ alkyl)NH₂, —NHSO₂CH₃, —N(CH₃)SO₂CH₃, —NHSO₂CH₂CH₃, —N(CH₃)SO₂CH₂CH₃, —SO₂NH₂, —CONH₂, —CON(C₁-C₃ alkyl)₂, —CONH(C₁-C₃ alkyl), —NHCO(C₁-C₃ alkyl), —NHCOO(C₁-C₃ alkyl), —N(CH₃)CO(C₁-C₃ alkyl), or —N(CH₃)COO(C₁-C₃ alkyl).

In one embodiment of the compounds of formula (II), (A), (A-I), (A-II), (A-III), (B), and/or (B-I), at least one R³ is —CH₂NHSO₂CH₃, —CH₂N(CH₃)SO₂CH₃, —NHSO₂CH₃, —NHSO₂CH₂CH₃, —SO₂NH₂, or —SO₂CH₃.

In one embodiment of the compounds of formula (II), (A), (A-I), (A-I), (A-II), (B), and/or (B-I), X is a bond, —CH₂—, —C(CH₃)₂—, or —C(═O)—. In one embodiment, X is a bond.

In one embodiment of the compounds of formula (II), (A), (A-I), (A-I), (A-II), (B), and/or (B-I), Y and Z are each —O—.

In one embodiment of the compounds of formula (I), (I-A), (I-B), (II), (A), (A-I), (A-I), (A-II), (B), and/or (B-I),
C is a 5- or 6-membered heteroaryl ring;
at least one R² is CN; and
at least one R³ is —NHSO₂CH₃, —NHSO₂CH₂CH₃, —SO₂NH₂, or —SO₂CH₃.

In embodiment of the compounds of formula (I), (I-A), (I-B), (II), (A), (A-I), (A-I), (A-II), (B), and/or (B-I),
C is a pyrimidine ring;
at least one R² is CN; and
at least one R³ is —NHSO₂CH₃, —NHSO₂CH₂CH₃, —SO₂NH₂, or —SO₂CH₃.

In one embodiment of the compounds of formula (I), (I-A), (I-B), (II), (A), (A-I), (A-I), (A-II), (B), and/or (B-I),
C is a pyrimidine ring;
at least one R² is CN; and
at least one R³ is —NHSO₂CH₃.

In one embodiment of the compound of formula (II) and/or (A), the compound is Compound A7, A8, A9, A10, A17, A18, A19, A20, A21, A22 A23, A24, A28, A29, A30, A31, A32, A33, A34, A35, A35(R), A35(S), A36, or A40, or a pharmaceutically acceptable salt, tautomer, stereoisomer or prodrug thereof. In one embodiment of the compound of formula (A), (A-I), or (A-II) the compound is Compound A7, A8, A9, A10, A17, A18, A19, A20, A21, A22 A23, A24, A28, A29, A30, A31, A32, A33, A34, A35, A35(R), A35(S), A36, A40, A44, A45, A46, A52, A53, A55, A56, A57, A58, A59, A60, A61, A62, A63, A64, A65, A69, A70, A71, A73, A74, A77, A81, A82, A83, A84, A85, A115, A122, A123, A124, A125, A126, A131, A133, A134, A135, A136, A137, A138, A139, A140, A143, A144, A146, A147, A148, A153, A154, A155, A156, A157, A158, A159, A160, A161, A162, A163, A164, A165, A167, A168, A169, A170, A171, A180, A185, A186, A187, A189, or A190, or a pharmaceutically acceptable salt, tautomer, stereoisomer or prodrug thereof. In one embodiment of the compound of formula (A-III), the compound is A72, A78, A79, A80, A128, A145, or A184, or a pharmaceutically acceptable salt, tautomer, stereoisomer or prodrug thereof.

In one embodiment of the compound of formula (II) and/or (B), the compound is Compound A3, A11, A12, A13, A14, A15, A16, A25, A26, A27, or A38, or a pharmaceutically acceptable salt, tautomer, stereoisomer or prodrug thereof. In one embodiment of the compound of formula (B) and/or (B-I), the compound is Compound A3, A11, A12, A13, A14, A15, A16, A25, A26, A27, A38, or A75, or a pharmaceutically acceptable salt, tautomer, stereoisomer or prodrug thereof.

In one embodiment, the present disclosure provides compounds of formula (C):

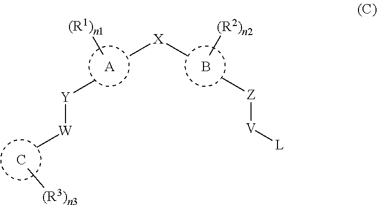

or a pharmaceutically acceptable salt, tautomer, stereoisomer or prodrug thereof, wherein:
A and B are each independently a phenyl ring;
C is a heteroaryl ring;
X is a bond, —CH₂—, —C(CH₃)₂—, or —C(═O)—;
Y is a bond or —O—;
W is —CH₂— or —C(CH₃)H—;
Z is a bond or —O—;
V is a phenylene;
L is halogen, —CHCl₂, —CCl₃, or —CF₃; or
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl).
n1 and n2 are each independently 0, 1, or 2; and
n3 is 1, 2, 3, 4 or 5.

In one embodiment, the present disclosure provides compounds of formula (D):

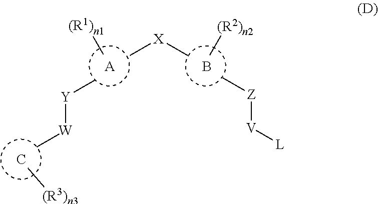

or a pharmaceutically acceptable salt, tautomer, stereoisomer or prodrug thereof, wherein:
A is absent;
B is 6- to 15-membered aryl ring;
C is a pyrimidine, triazine, or thiophene ring;
X is a bond, —CH₂—, —C(CH₃)₂—, or —C(═O)—;
Y is a bond or —O—;
W is —CH₂— or —C(CH₃)H—;
Z is a bond or —O—;
V is —CH₂— and L is halogen, —CHCl₂, —CCl₃, or —CF₃; or
V is —CH₂CH₂— and L is halogen;
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
n1 and n2 are each independently 0, 1, or 2; and
n3 is 1, 2, 3, 4 or 5.

In one embodiment of the compounds of formula (D), B is phenyl or fluorene ring, wherein each ring is optionally substituted with one or two R².

In one embodiment, the present disclosure provides compounds of formula (E):

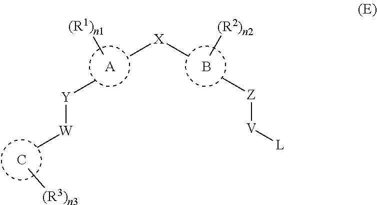

or a pharmaceutically acceptable salt, tautomer, stereoisomer or prodrug thereof, wherein:
A is a 5- or 6-membered heterocyclyl or heteroaryl;
B is phenyl;
C is a heteroaryl ring;
X is a bond, —CH₂—, —C(CH₃)₂—, or —C(═O)—;
Y is a bond or —O—;
W is —CH₂— or —C(CH₃)H—;
Z is a bond or —O—;
V is —CH₂— and L is halogen, —CHCl₂, —CCl₃, or —CF₃; or
V is —CH₂CH₂— and L is halogen;
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
n1 and n2 are each independently 0, 1, or 2; and
n3 is 1, 2, 3, 4 or 5.

In one embodiment of the compounds of formula (E), the compound is not

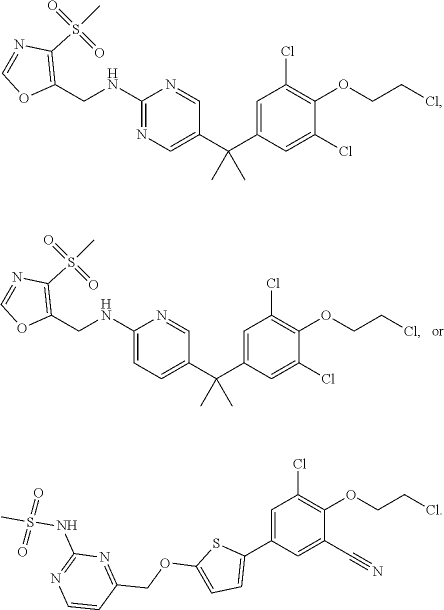 [SMILES: CC(C)(c1cnc(NCc2ocnc2S(C)(=O)=O)nc1)c1cc(Cl)c(OCCCl)c(Cl)c1 ; CC(C)(c1ccc(NCc2ocnc2S(C)(=O)=O)nc1)c1cc(Cl)c(OCCCl)c(Cl)c1 ; CS(=O)(=O)Nc1nccc(COc2ccc(-c3cc(Cl)c(OCCCl)c(C#N)c3)s2)n1]

In one embodiment of the compounds of formula (E), C is a pyrimidine.

In one embodiment, the present disclosure provides compounds of formula (E-I):

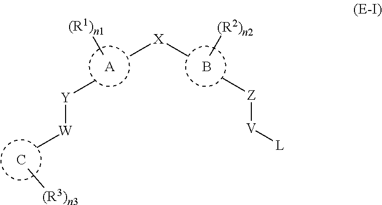

or a pharmaceutically acceptable salt, tautomer, stereoisomer or prodrug thereof, wherein:
A is a 5- or 6-membered heterocyclyl or heteroaryl;
B is phenyl;
C is a 6- to 12-membered heteroaryl ring;
X is a bond, —CH₂—, —CH(CH₃)—, —C(CH₃)₂—, or —C(═O)—;
Y is a bond, —O—, or —NH—;
W is a bond, —CH₂— or —C(CH₃)H—;
Z is a bond —NH—, or —O—;
V is —CH₂— and L is halogen, —CHCl₂, —CCl₃, or —CF₃; or
V is —CH₂CH₂— and L is halogen;
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —NHCOO(C₁-C₃ alkyl), —N(CH₃)CO(C₁-C₃ alkyl), or —N(CH₃)COO(C₁-C₃ alkyl);
n1 and n2 are each independently 0, 1, or 2; and
n3 is 1, 2, 3, or 4;
wherein A is not thiophene.

In one embodiment, the present disclosure provides compounds of formula (E-I), C is a fused bicyclic heteroaryl ring. In one embodiment, C is any fused bicyclic heteroaryl as described for formula (G).

In one embodiment, the present disclosure provides compounds of formula (E-I), C is

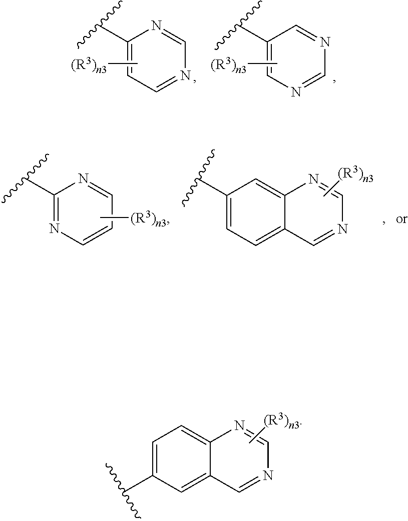

In one embodiment of the compounds of formula (E) and/or (E-I), A is piperidine, pyrazole, or oxadiazole ring, wherein each ring is optionally substituted with one or two R¹.

In one embodiment, the present disclosure provides compounds of formula (F):

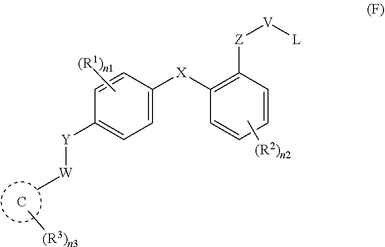

or a pharmaceutically acceptable salt, tautomer, stereoisomer or prodrug thereof, wherein:
A is a 5- or 6-membered heterocyclyl or heteroaryl;
B is phenyl;
C is a heteroaryl ring;
X is a bond, —CH₂—, —C(CH₃)₂—, or —C(═O)—;
Y is a bond or —O—;
W is —CH₂— or —C(CH₃)H—;
Z is a bond or —O—;
V is —CH₂— and L is halogen, —CHCl₂, —CCl₃, or —CF₃; or
V is —CH₂CH₂— and L is halogen;
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
n1 and n2 are each independently 0, 1, or 2; and
n3 is 1, 2, 3, 4 or 5.

In one embodiment, the present disclosure provides compounds of formula (G):

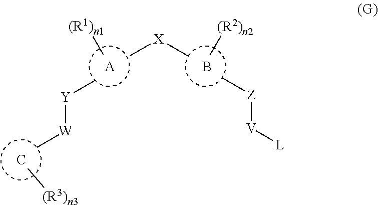

or a pharmaceutically acceptable salt, tautomer, stereoisomer or prodrug thereof, wherein:
A and B are each independently a phenyl ring;
C is a fused or a bridged bicyclic 5- to 15-membered ring selected from aryl, carbocyclyl, heteroaryl, or heterocyclyl;
X is a bond, —CH₂—, —C(CH₃)₂—, or —C(═O)—;
Y is a bond, —CH₂—, —NH—, or —O—;
W is a bond, —CH₂—, —CH₂CH₂—, —C(CH₃)H—, —N(R⁷)CO—, or —CONR⁷—;
Z is a bond —NH—, or —O—;
V is a bond, —CH₂(CR^{8a′}R^{9a′})—, or —(CR^{8a}R^{9a})ₘ—;
L is hydrogen, halogen, —CF₂R¹⁰, —CF₃, —CN, —OR¹⁰, —NR¹¹R¹², or —CONR¹¹R¹²;
R¹ and R² are each independently halogen, —CN, or —CF₃;
at least one R³ is —CH₂NHSO₂CH₃, —CH₂N(CH₃)SO₂CH₃, —NHSO₂CH₃, —NHSO₂CH₂CH₃, —SO₂NH₂, —SO₂CH₃, —NH₂, or —NH(C₁-C₃ alkyl); and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), or —N(CH₃)COO(C₁-C₃ alkyl);
R⁷ is hydrogen or C₁-C₃ alkyl;
R^{8a} and R^{9a} are each independently hydrogen, halogen, or C₁-C₃ alkyl;
R^{8a′} and R^{9a′} taken together form an optionally substituted 3- to 6-membered carbocyclyl or heterocyclyl containing one, two, or three heteroatoms selected from N, S, or O;
R¹⁰ is hydrogen, or C₁-C₃ alkyl;
R¹¹ and R¹² are each independently hydrogen or C₁-C₃ alkyl; or R¹¹ and R¹² taken together form an optionally substituted heterocyclyl which optionally contains one or two additional heteroatoms selected from N, S, or O;
m is 0, 1, or 2;
n1 and n2 are each independently 0, 1, or 2; and
n3 is 1, 2, 3, 4 or 5;
where C is not

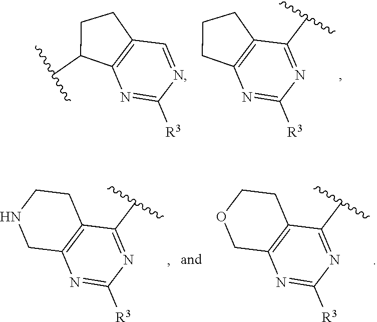

In one embodiment of the compounds of formula (G), C is a bridged bicyclic carbocycle, 6,6-fused heteroaryl or heterocycle, 5,6-fused heteroaryl or heterocycle, 6,5-fused heteroaryl or heterocycle, or 5,5-fused heteroaryl or heterocycle. In one embodiment, C is a 6,6-fused heteroaryl or heterocycle, 5,6-fused heteroaryl or heterocycle, 6,5-fused heteroaryl or heterocycle, or 5,5-fused heteroaryl or heterocycle.

In one embodiment of the compounds of formula (G), C is

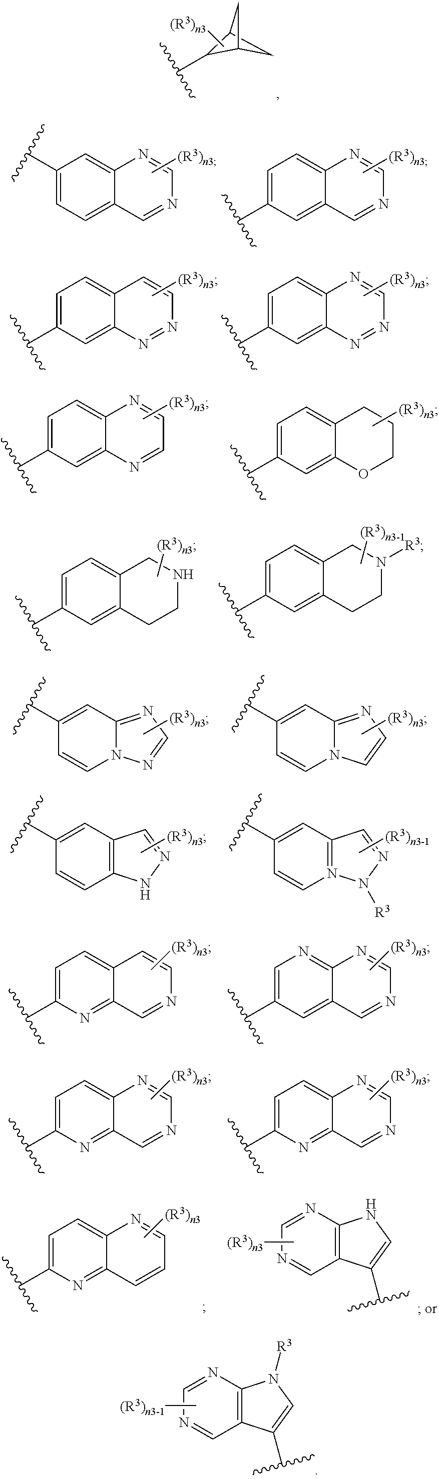

In one embodiment, the present disclosure provides compounds of formula (H):

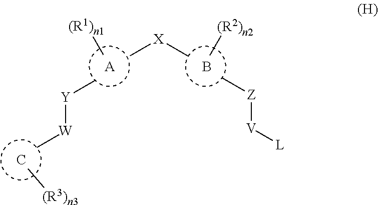

or a pharmaceutically acceptable salt, tautomer, stereoisomer or prodrug thereof, wherein:
A and B are each independently a phenyl or a pyridyl ring;
C is 5- or 6-membered heteroaryl, 4- to 6-membered heterocyclyl, phenyl, or bicyclic 5- or 6-membered carbocycle;
X is a bond, —CH₂—, —C(CH₃)₂—, or —C(═O)—;
Y is a bond, —CH₂—, —NH—, or —O—;
W is —CH₂—, —CH₂CH₂—, —C(CH₃)H—, —N(R⁷)CO—, or —CONR⁷—;
Z is a bond, —NH—, or —O—;
V is a bond, —CH₂(CR^{8a′}R^{9a′})—, or —(CR^{8a}R^{9a})ₘ—;
L is hydrogen, halogen, —CF₂R¹⁰, —CF₃, —CN, —OR¹⁰, —NR¹¹R¹², or —CONR¹¹R¹²;
R¹ and R² are each independently halogen, —CN, or —CF₃;
at least one R³ is —CH₂NHSO₂CH₃, —CH₂N(CH₃)SO₂CH₃, —NHSO₂CH₃, —NHSO₂CH₂CH₃, —SO₂NH₂, —SO₂CH₃, —NH₂, —NH(C₁-C₃ alkyl), —NHCOCF₃ or optionally substituted 5- or 6-membered heterocyclyl or heteroaryl; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), —N(CH₃)COO(C₁-C₃ alkyl); or optionally substituted 5- or 6-membered heterocyclyl;
R⁷ is hydrogen or C₁-C₃ alkyl;
R^{8a} and R^{9a} are each independently hydrogen, halogen, or C₁-C₃ alkyl;
R^{8a′} and R^{9a′} taken together form an optionally substituted 3- to 6-membered carbocyclyl or heterocyclyl containing one, two, or three heteroatoms selected from N, S, or O;
R¹⁰ is hydrogen, or C₁-C₃ alkyl;
R¹¹ and R¹² are each independently hydrogen or C₁-C₃ alkyl; or R¹¹ and R¹² taken together form an optionally substituted heterocyclyl which optionally contains one or two additional heteroatoms selected from N, S, or O;
m is 0, 1, or 2;
n1 and n2 are each independently 0, 1, or 2; and
n3 is 1, 2, 3, 4 or 5;
wherein:
a) at least one R³ is optionally substituted 4- to 6-membered heterocyclyl or heteroaryl, provided that 5- or 6-membered heterocyclyl is not

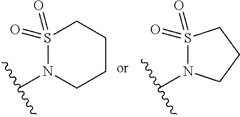 [SMILES: *N1CCCCS1(=O)=O ; *N1CCCS1(=O)=O]

when R³ is pyrimidyl; or
b) Z is a bond; V is a bond; L is —NR¹¹R¹²; and R¹¹ and R¹², taken together with the nitrogen atom to which it is attached, form an optionally substituted heterocyclic ring.

In one embodiment of the compounds of formula (H), C is a pyrimidine, pyrazine, pyridine, pyrazole, azetidine, phenyl, or bicyclo[1.1.1]pentane.

In one embodiment of the compounds of formula (H), R¹¹ and R¹², taken together with the nitrogen atom to which it is attached, form an optionally substituted piperidine or 2-azaspiro[3.3]heptane.

In one embodiment of the compounds of formula (H), at least one R³ is optionally substituted 4- or 6-membered heterocyclyl or heteroaryl containing one or two heteroatoms selected from N and O. In one embodiment, at least one R³ is an optionally substituted group selected from: pyrazolyl, morpholinyl, piperazinyl, piperidinyl, pyrrolidinyl, or azetidinyl. In one embodiment, at least one R³ is substituted with —OH, oxo, —CH₂NHSO₂CH₃, —CH₂N(CH₃)SO₂CH₃, —NHSO₂CH₃, —NHSO₂CH₂CH₃, —SO₂NH₂, —SO₂CH₃, —NH₂, —NH(C₁- C₃ alkyl), or —NHCOCF₃.

In one embodiment of the compounds of formula (H), Z is —NH— or —O—; V is —(CR^{8a}R^{9a})ₘ—; and L is halogen or H.

In one embodiment of the compounds of formula (G) and/or (H):
Z is a bond —NH—, or —O—; and
V is —CH₂— and L is halogen, —CHCl₂, —CCl₃, or —CF₃; or
V is —CH₂CH₂— and L is halogen.

In one embodiment, the present disclosure provides compounds of formula (J):

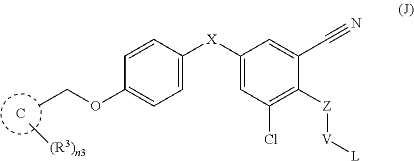

or a pharmaceutically acceptable salt, tautomer, stereoisomer or prodrug thereof, wherein:
C is pyrimidyl;
X is a bond, —CH₂— or —C(CH₃)₂—;
Z is —O— and —V-L is —CH₂CH(OH)CH₃, —CH₂CH(OH)CH₂Cl, or —CH₂CH₂CH₂Cl; or
Z is —NH—, V is —(CR^{8a}R^{9a})ₘ—, and L is hydrogen or halogen;
at least one R³ is —CH₂NHSO₂CH₃, —CH₂N(CH₃)SO₂CH₃, —NHSO₂CH₃, —NHSO₂CH₂CH₃, —SO₂NH₂, —SO₂CH₃, —NH₂, —NH(C₁-C₃ alkyl), —NHCOCF₃ or optionally substituted 5- or 6-membered heterocyclyl or heteroaryl; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), —N(CH₃)COO(C₁-C₃ alkyl); or optionally substituted 5- or 6-membered heterocyclyl;
R^{8a} and R^{9a} are each independently hydrogen, halogen, or C₁-C₃ alkyl;
m is 1, or 2; and
n3 is 1, 2, 3, 4 or 5.

In one embodiment, the present disclosure provides compounds of formula (K):

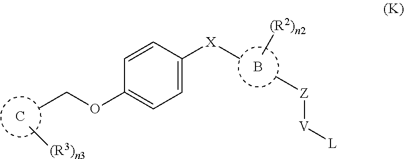

or a pharmaceutically acceptable salt, tautomer, stereoisomer or prodrug thereof, wherein:
B is pyridyl;
C is pyrimidyl;
X is a bond, —CH₂—, —C(CH₃)₂—, or —C(═O)—;
Z is a bond, —NH—, or —O—;
V is a bond, —CH₂(CR^{8a′}R^{9a′})—, or —(CR^{8a}R^{9a})ₘ—;
L is hydrogen, halogen, —CF₂R¹⁰, —CF₃, —CN, —OR¹⁰, —NR¹¹R¹², or —CONR¹¹R¹²;
R² is each independently halogen, —CN, or —CF₃;
at least one R³ is —CH₂NHSO₂CH₃, —CH₂N(CH₃)SO₂CH₃, —NHSO₂CH₃, —NHSO₂CH₂CH₃, —SO₂NH₂, —SO₂CH₃, —NH₂, —NH(C₁-C₃ alkyl), —NHCOCF₃ or optionally substituted 5- or 6-membered heterocyclyl or heteroaryl; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), —N(CH₃)COO(C₁-C₃ alkyl); or optionally substituted 5- or 6-membered heterocyclyl;
R^{8a} and R^{9a} are each independently hydrogen, halogen, or C₁-C₃ alkyl;
R^{8a′} and R^{9a′} taken together form an optionally substituted 3- to 6-membered carbocyclyl or heterocyclyl containing one, two, or three heteroatoms selected from N, S, or O;
R¹⁰ is hydrogen, or C₁-C₃ alkyl;
R¹¹ and R¹² are each independently hydrogen or C₁-C₃ alkyl; or R¹¹ and R¹² taken together form an optionally substituted heterocyclyl which optionally contains one or two additional heteroatoms selected from N, S, or O;
m is 1, 2, or 3;
n2 is 0, 1, or 2; and
n3 is 1, 2, 3, 4 or 5.

In one embodiment of the compounds of formula (K), n2 is 1 and R² is halogen.

In one embodiment of the compounds of formula (I), (I-A), (I-B), (II), (A), (A-I), (A-II), (A-III), (B), (B-I), (C), (D), (E), (E-I), (F), (G), (H), and/or (K), C is a pyrimidine, triazine, or thiophene ring. In one embodiment of the compounds of formula (I), (I-A), (I-B), (II), (A), (A-I), (A-II), (A-III), (B), (B-I), (C), (D), (E), (E-I), (F), (G), (H), and/or (K), C is a pyrimidine ring.

In one embodiment of the compounds of formula (C), (D), (E), (E-I), (F), (G), (H), and/or (K), R¹ and R² are each independently Cl, —CN, or —CF₃.

In one embodiment of the compounds of formula (C), (D), (E), (E-I), (F), (G), (H), (J), and/or (K), at least one R³ is —NHSO₂CH₃, —NHSO₂CH₂CH₃, —SO₂NH₂, or —SO₂CH₃. In one embodiment, at least one R³ is —NHSO₂CH₃, —NHSO₂CH₂CH₃, —SO₂NH₂, or —SO₂CH₃ and the other R³ is, if present, —CN, C₁-C₃ alkyl, C₁-C₃ alkoxy, —SO₂(C₁-C₃ alkyl), —NH₂, —(C₁-C₃ alkyl)NH₂, —NHSO₂CH₃, —N(CH₃)SO₂CH₃, —NHSO₂CH₂CH₃, —N(CH₃)SO₂CH₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl).

In one embodiment of the compounds of formula (C), (D), (E), (E-I), (F), (G), (H), (J), and/or (K) at least one R³ is —CH₂NHSO₂CH₃, —CH₂N(CH₃)SO₂CH₃, —NHSO₂CH₃, —NHSO₂CH₂CH₃, —SO₂NH₂, —SO₂CH₃, —NH₂, —NH(C₁-C₃ alkyl), or —NHCOCF₃; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), —N(CH₃)COO(C₁-C₃ alkyl); or optionally substituted 5- or 6-membered heterocyclyl.

In one embodiment of the compound of formula (II) and/or (C), the compound is Compound A1 or A2, or a pharmaceutically acceptable salt, tautomer, stereoisomer or prodrug thereof.

In one embodiment of the compound of formula (II) and/or (D), the compound is Compound A4 or A5, or a pharmaceutically acceptable salt, tautomer, stereoisomer or prodrug thereof.

In one embodiment of the compound of formula (II) and/or (E), the compound is Compound A6, A37, or A41, or a pharmaceutically acceptable salt, tautomer, stereoisomer or prodrug thereof. In one embodiment of the compound of formula (E) and/or (E-I), the compound is Compound A6, A37, A41, A141, A142, A182, A183, or A188, or a pharmaceutically acceptable salt, tautomer, stereoisomer or prodrug thereof.

In one embodiment of the compound of formula (II) and/or (F), the compound is Compound A39 or a pharmaceutically acceptable salt, tautomer, stereoisomer or prodrug thereof.

In one embodiment of the compound of formula (G), the compound is Compound A51, A107, A108, A109, A111, A112, A113, A114, A117, A121, A132, A149, A152, A172, A173, A175, A176, A177, A191, A192, A193, A194, A195, A196, A197, A198, or A199, or a pharmaceutically acceptable salt, tautomer, stereoisomer or prodrug thereof.

In one embodiment of the compound of formula (H), the compound is Compound A42, A90, A99, A100, A101, A105, A106, A118, A119, A120, A151, A166, A178, or A179, or a pharmaceutically acceptable salt, tautomer, stereoisomer or prodrug thereof.

In one embodiment of the compound of formula (J), the compound is Compound A66, A129, A130, A150, A174, or A181, or a pharmaceutically acceptable salt, tautomer, stereoisomer or prodrug thereof.

In one embodiment of the compound of formula (K), the compound is Compound A67, A103, or A104, or a pharmaceutically acceptable salt, tautomer, stereoisomer or prodrug thereof.

In one embodiment, the present disclosure further provides compounds disclosed in Table A, or a pharmaceutically acceptable salt, tautomer, stereoisomer or prodrug thereof.

In one embodiment of the compounds of the present disclosure, compounds disclosed in WO 2020/081999 is excluded.

The present disclosure further provides a pharmaceutical composition comprising a compound of formula (I), (I-A), (I-B), (II), (A), (A-I), (A-II), (A-III), (B), (B-I), (C), (D), (E), (E-I), (F), (G), (H), (J), and/or (K), and a pharmaceutically acceptable carrier.

The present disclosure further provides a method for treating cancer, comprising administering the compound, pharmaceutically acceptable salt, tautomer, stereoisomer or prodrug of the compound of formula (I), (I-A), (I-B), (II), (A), (A-I), (A-II), (A-III), (B), (B-I), (C), (D), (E), (E-I), (F), (G), (H), (J), and/or (K), to a subject in need thereof.

In one embodiment of the methods disclosed herein, cancer is prostate cancer. In one embodiment, the prostate cancer is metastatic castration-resistant prostate cancer. In one embodiment, the prostate cancer expresses full-length androgen receptor or truncated androgen receptor splice variant.

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in the art, the following definitions are set forth to facilitate explanation of the presently disclosed subject matter.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “a androgen receptor modulator” refers to one or more androgen receptor modulators or at least one androgen receptor modulator. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The compounds of the invention, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

A “tautomer” refers to a proton shift from one atom of a molecule to another atom of the same molecule. The present disclosure includes tautomers of any said compounds.

A “prodrug” refers to a derivative of a compound of the present disclosure that will be converted to the compound in vivo. In one embodiment of the present disclosure, a prodrug includes a compound of formula (I), (I-A), (I-B), (II), (A), (A-I), (A-II), (A-III), (B), (B-I), (C), (D), (E), (E-I), (F), (G), (H), (J), and (K), having a free hydroxyl group (—OH) that is acetylated (—OCOMe) at one or more positions.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof.

As used herein, the term “pharmaceutical composition” refers to a formulation comprising at least one therapeutically active agent and a pharmaceutically acceptable excipient or carrier. A non-limiting example of pharmaceutical compositions includes tablets, capsules, gel capsules, syrup, liquid, gel, suspension, solid dispersion, or combinations thereof.

As used herein, the term “dosage form” refers to one or more pharmaceutical compositions which provides a specific amount of a therapeutically active agent, such as a unit dose.

As used herein, a “subject” can be a human, non-human primate, mammal, rat, mouse, cow, horse, pig, sheep, goat, dog, cat and the like. The subject can be suspected of having or at risk for having a cancer, such as prostate cancer, breast cancer, ovarian cancer, salivary gland carcinoma, or endometrial cancer, or suspected of having or at risk for having acne, hirsutism, alopecia, benign prostatic hyperplasia, ovarian cysts, polycystic ovary disease, precocious puberty, spinal and bulbar muscular atrophy, or age-related macular degeneration. Diagnostic methods for various cancers, such as prostate cancer, breast cancer, ovarian cancer, bladder cancer, pancreatic cancer, hepatocellular cancer, salivary gland carcinoma, or endometrial cancer, and diagnostic methods for acne, hirsutism, alopecia, benign prostatic hyperplasia, ovarian cysts, polycystic ovary disease, precocious puberty, spinal and bulbar muscular atrophy, or age-related macular degeneration and the clinical delineation of cancer, such as prostate cancer, breast cancer, ovarian cancer, bladder cancer, pancreatic cancer, hepatocellular cancer, salivary gland carcinoma, or endometrial cancer, diagnoses and the clinical delineation of acne, hirsutism, alopecia, benign prostatic hyperplasia, ovarian cysts, polycystic ovary disease, precocious puberty, spinal and bulbar muscular atrophy, or age-related macular degeneration are known to those of ordinary skill in the art.

“Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

All weight percentages (i.e., “% by weight” and “wt. %” and w/w) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

The terms below, as used herein, have the following meanings, unless indicated otherwise:

“Amino” refers to the —NH₂ radical.

“Cyano” refers to the —CN radical.

“Halo” or “halogen” refers to bromo, chloro, fluoro or iodo radical, including their radioisotopes. “¹²³I” refers to the radioactive isotope of iodine having atomic mass 123. The compounds of Formula I can comprise at least one ¹²³I moiety. Throughout the present application, where structures depict a ¹²³I moiety at a certain position it is meant that the I moiety at this position is enriched for ¹²³I. In other words, the compounds contain more than the natural abundance of ¹²³I at the indicated position(s). It is not required that the compounds comprise 100% ¹²³I at the indicated positions, provided ¹²³I is present in more than the natural abundance. Typically, the ¹²³I isotope is enriched to greater than 50%, greater than 60%, greater than 70%, greater than, 80% or greater than 90%, relative to ¹²⁷. “¹⁸F” refers to the radioactive isotope of fluorine having atomic mass 18. “F” or “¹⁹F” refers to the abundant, non-radioactive fluorine isotope having atomic mass 19. The compounds of Formula I can comprise at least one ¹⁸F moiety. Throughout the present application, where structures depict a ¹⁸F moiety at a certain position it is meant that the F moiety at this position is enriched for ¹⁸F. In other words, the compounds contain more than the natural abundance of ¹⁸F at the indicated position(s). It is not required that the compounds comprise 100% ¹⁸F at the indicated positions, provided ¹⁸F is present in more than the natural abundance. Typically, the ¹⁸F isotope is enriched to greater than 50%, greater than 60%, greater than 70%, greater than 80% or greater than 90%, relative to ¹⁹F.

“Hydroxy” or “hydroxyl” refers to the —OH radical.

“Imino” refers to the ═NH substituent.

“Nitro” refers to the —NO₂ radical.

“Oxo” refers to the =0 substituent.

“Thioxo” refers to the ═S substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkynyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alknyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “Cw-Cz acyl” where w and z depicts the range of the number of carbon atoms in Rₐ, as defined above. For example, “C₁-C₁₀ acyl” refers to alkylcarbonyl group as defined above, where Rₐ is C₁-C₁₀ alkyl, C₁-C₁₀ alkenyl, or C₁-C₁₀ alkynyl radical as defined above. Unless stated otherwise specifically in the specification, an alkyl carbonyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” or “arylalkyl” refers to a radical of the formula —R_{b}—R_{c} where R_{b} is an alkylene group as defined above and R, is one or more aryl radicals as defined above, for example, benzyl, diphenylmethyl and the like. Unless stated otherwise specifically in the specification, an aralkyl group can be optionally substituted.

“Aralkenyl” or “arylalkenyl” refers to a radical of the formula —R_{b}—R_{c} where R_{b} is an alkenylene o group as defined above and R_{c} is one or more aryl radicals as defined above. Unless stated otherwise specifically in the specification, an aralkenyl group can be optionally substituted.

“Aralkynyl” or “arylalkynyl” refers to a radical of the formula —R_{b}—R_{c} where R_{b} is an alkynylene group as defined above and R_{c} is one or more aryl radicals as defined above. Unless stated otherwise specifically in the specification, an aralkynyl group can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include aryls and cycloalkyl. cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkenyl group can be optionally substituted.

“Heterocyclyl,” “heterocyclic ring” or “heterocycle” refers to a stable 3- to 20-membered non-aromatic, partially aromatic, or aromatic ring radical which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heterocyclylalkyl” refers to a radical of the formula —R_{b}—Rₑ where R_{b} is an alkylene group as defined above and Re is a heterocyclyl radical as defined above. Unless stated otherwise specifically in the specification, a heterocycloalkylalkyl group can be optionally substituted.

“Heterocyclylalkenyl” refers to a radical of the formula —R_{b}—Rₑ where R_{b} is an alkenylene group as defined above and Re is a heterocyclyl radical as defined above. Unless stated otherwise specifically in the specification, a heterocycloalkylalkenyl group can be optionally substituted.

“Heterocyclylalkynyl” refers to a radical of the formula —R_{b}—Rₑ where R_{b} is an alkynylene group as defined above and Re is a heterocyclyl radical as defined above. Unless stated otherwise specifically in the specification, a heterocycloalkylalkynyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophene), benzotriazolyl, benzo[4,6]imidazo[1,2-a]pyridinyl, carbazolyl, cinnolinyl, dibenzofuranyl, dibenzothiophene, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophene (i.e. thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}—R_{f} where R_{b} is an alkylene chain as defined above and R_{f} is a heteroaryl radical as defined above. Unless stated otherwise specifically in the specification, a heteroarylalkyl group can be optionally substituted.

“Heteroarylalkenyl” refers to a radical of the formula —R_{b}—R_{f} where R_{b} is an alkenylene, chain as defined above and R_{f} is a heteroaryl radical as defined above. Unless stated otherwise specifically in the specification, a heteroarylalkenyl group can be optionally substituted.

“Heteroarylalkynyl” refers to a radical of the formula —R_{b}—R_{f} where R_{b} is an alkynylene chain as defined above and R_{f} is a heteroaryl radical as defined above. Unless stated otherwise specifically in the specification, a heteroarylalkynyl group can be optionally substituted.

“Ring” refers to a cyclic group which can be fully saturated, partially saturated, or fully unsaturated. A ring can be monocyclic, bicyclic, tricyclic, or tetracyclic. Unless stated otherwise specifically in the specification, a ring can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (i.e., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups.

“Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

[symbol]

As used herein, the symbol “ [symbol] ” (hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example, “ [symbol] ” indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or “ [symbol] ” infers that when R³ is “XY”, the point of attachment bond is the same bond as the bond by which R³ is depicted as being bonded to CH₃. When a group is divalent which can be denoted as having two points of attachment such as

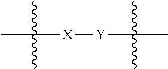

or —X—Y—, unless it is expressly stated, the divalent group can attach in either direction. That is, if the above divalent group (—X—Y—) represents U in T-U—V, then it can be T-X—Y—V or T-Y—X-V.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring can be replaced with a nitrogen atom.

The following description includes information that may be useful in understanding the present invention. It is not an admission that any of the information provided herein is prior art or relevant to the presently claimed inventions, or that any publication specifically or implicitly referenced is prior art.

### Compounds of the Present Disclosure

The compound of the present disclosure can be useful for modulating androgen receptor (AR). Further, the compound of the present disclosure can be useful for treating various diseases and conditions including, but not limited to, cancer. In some embodiments, the cancer is prostate cancer or breast cancer.

In one embodiment, the present disclosure provides compounds comprising the structure of formula (I):

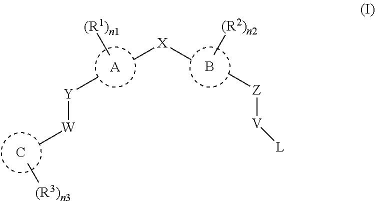

or a pharmaceutically acceptable salt, tautomer, stereoisomer or prodrug thereof, wherein:
A and B are each independently a 4- to 15-membered ring selected from aryl, carbocyclyl, heteroaryl, or heterocyclyl;
C is a 3- to 10-membered ring;
X is a bond, —(CR⁵R⁶)ₜ—, —O—, —C(═O)—, —S—, —S(═O)—, —SO₂—, —NR⁷—, —N(R⁷)CO—, —CON(R⁷)—, or —NSO₂R⁷—;
Y and Z are each independently a bond, —(CR⁸R⁹)ₘ—, —O—, —C(═O)—, —S—, —S(═O)—, —SO₂—, or —NR⁷—;
W and V are each independently a bond, —(CR^{8a}R^{9a})ₘ—, phenylene, —C(═O)—, —N(R⁷)CO—, —CONR⁷—, or —NSO₂R⁷—;
L is hydrogen, halogen, —CF₂R¹⁰, —CF₃, —CN, —OR¹⁰; —NR¹¹R¹², or —CONR¹¹R¹²;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, oxo, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;
R³ is hydrogen, halogen, oxo, ═S, ═NR¹⁶, —CN, —CF₃, —OH, —SR¹⁶, optionally substituted C₁-C₆ alkyl, optionally substituted C₂-C₆ alkenyl, optionally substituted C₂-C₆ alkynyl, optionally substituted C₁-C₆ alkoxy, optionally substituted —(C₁-C₆ alkyl)-(C₁-C₆ alkoxy), optionally substituted —(C₁-C₆ alkyl)-OH, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, —NR¹⁴COOR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶, optionally substituted —(C₁-C₆ alkyl)-SO₂R¹⁶, optionally substituted carbocyclyl, optionally substituted heterocyclyl, optionally substituted aryl, or optionally substituted heteroaryl;
R⁵ and R⁶ are each independently hydrogen, halogen, —OH, optionally substituted C₁-C₆ alkyl, optionally substituted C₂-C₆ alkenyl, optionally substituted C₂-C₆ alkynyl, optionally substituted C₁-C₆ alkoxy, —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl; or R⁵ and R⁶ taken together form an optionally substituted carbocyclyl or optionally substituted heterocyclyl;
R⁸ and R⁹ are each independently hydrogen, halogen, or C₁-C₃ alkyl;
R^{8a} and R^{9a} are each independently hydrogen, —OH, halogen, optionally substituted C₁-C₆ alkyl, optionally substituted C₂-C₆ alkenyl, optionally substituted C₂-C₆ alkynyl, optionally substituted C₁-C₆ alkoxy, optionally substituted —OCO(C₁-C₆ alkyl), —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl; or R^{8a} and R^{9a} taken together form an optionally substituted carbocyclyl or optionally substituted heterocyclyl;
R⁷, R¹⁰ and R¹⁶ are each independently hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl; or R⁷ and R^{8a} taken together form an optionally substituted heterocyclyl;
R¹¹, R¹², R¹³, R¹⁴ and R¹⁵ are each independently hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl; or (R¹¹ and R¹²) or (R¹⁴ and R¹⁵) taken together form an optionally substituted heterocyclyl;
each m is independently 0, 1 or 2;
n1 and n2 are each independently 0, 1, 2, 3, or 4;
n3 is 0, 1, 2, 3, 4 or 5; and
each t is independently 0, 1 or 2.

In one embodiment, the present disclosure provides compounds comprising the structure of formula (I-A):

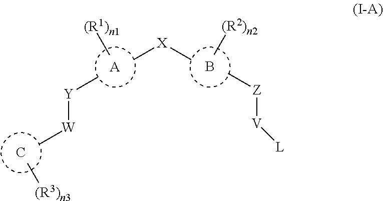

or a pharmaceutically acceptable salt, tautomer, stereoisomer or prodrug thereof, wherein:
A is a 4- to 15-membered ring selected from aryl, carbocyclyl, heteroaryl, or heterocyclyl;
B is a 4- to 15-membered ring selected from aryl, carbocyclyl, heteroaryl, or heterocyclyl;
C is a 3- to 10-membered aryl, carbocyclyl, heteroaryl, or heterocyclyl;
X is a bond, —(CR⁵R⁶)ₜ—, —C(═O)—, or —NR⁷;
Y is a bond, —(CR⁸R⁹)ₘ—, —O—, —S—, —S(═O)—, —SO₂—, —NR⁷—, or —N(COCH₃)—;
W is a bond, —(CR^{8a}R^{9a})ₘ—, —C(═O)—, —NR⁷—, —N(R⁷)CO—, —CONR⁷—, or —NSO₂R⁷—;
Z is a bond, —CH₂—, —C(CH₃)H—, —O—, —S—, —NH—, —NCH₃—, or —N(COCH₃)—;
V is a bond, —(CR^{8a}R^{9a})ₘ—, —CR^{8a}═CR^{9a}—, or phenylene;
L is hydrogen, halogen, —CF₂R¹⁰, —CF₃, —CN, —OR¹⁰, —SO₂R¹⁷, —NR¹¹R¹², or —CONR¹¹R¹²;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, oxo, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —NR¹⁴C₀₀R¹⁶, —(C₁-C₆ alkyl)-NR¹⁴COR¹⁶, —CONR¹⁴R¹⁵, —(C₁-C₃ alkyl)-CONR¹⁴R⁵, —SO₂NR¹⁴R¹⁵, —(C₁-C₃ alkyl)-SO₂NR¹⁴R⁵, —SO₂(C₁-C₃ alkyl), —(C₁-C₆ alkyl)-SO₂(C₁-C₃ alkyl), optionally substituted carbocyclyl, optionally substituted heterocyclyl, optionally substituted aryl, or optionally substituted heteroaryl;
R⁵ and R⁶ are each independently hydrogen, halogen, —OH, optionally substituted C₁-C₃ alkyl, optionally substituted C₂-C₃ alkenyl, optionally substituted C₂-C₃ alkynyl, or C₁-C₃ alkoxy; or R⁵ and R⁶ taken together form an optionally substituted 3- to 6-membered carbocyclyl or heterocyclyl;
R⁷ is hydrogen, C₁-C₆ alkyl, C₂-C₆ alkenyl, or C₂-C₆ alkynyl;
R⁸ and R⁹ are each independently hydrogen, halogen, or C₁-C₃ alkyl;
R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{9a} taken together form an optionally substituted 3- to 6-membered carbocyclyl or heterocyclyl;
R¹⁰ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl;
R¹¹ and R¹² are each independently hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl; or R¹¹ and R¹² taken together form an optionally substituted heterocyclyl which optionally contains one or two additional heteroatoms selected from N, S, or O;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form an optionally substituted 3- to 6-membered heterocyclyl;
R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;
R¹⁷ is hydrogen, optionally substituted C₁-C₃ alkyl, optionally substituted C₂-C₃ alkenyl, or optionally substituted C₂-C₃ alkynyl;
each m is independently 0, 1, or 2;
n1 and n2 are each independently 0, 1, or 2;
n3 is 0, 1, 2, 3, 4 or 5; and
t is 0, 1 or 2.

In one embodiment, the present disclosure provides compounds comprising the structure of formula (I-B):

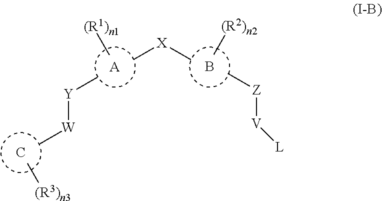

or a pharmaceutically acceptable salt, tautomer, stereoisomer or prodrug thereof, wherein:
A is a 4- to 15-membered ring selected from aryl, carbocyclyl, heteroaryl, or heterocyclyl;
B is a 4- to 15-membered ring selected from aryl, carbocyclyl, heteroaryl, or heterocyclyl;
C is a 3- to 10-membered aryl, carbocyclyl, heteroaryl, or heterocyclyl;
X is a bond, —(CR⁵R⁶)ₜ—, —C(═O)—, or —NR⁷;
Y is a bond, —(CR⁸R⁹)ₘ—, —O—, —S—, —S(═O)—, —SO₂—, —NR⁷—, or —N(COCH₃)—;
W is a bond, —(CR^{8a}R^{9a})ₘ—, —C(═O)—, —N(R⁷)CO—, —CONR⁷—, or —NSO₂R⁷—;
Z is a bond, —CH₂—, —C(CH₃)H—, —O—, —S—, —NH—, —NCH₃—, or —N(COCH₃)—;
V is a bond, —(CR^{8a}R^{9a})ₘ—, or phenylene;
L is hydrogen, halogen, —CF₂R¹⁰, —CF₃, —CN, —OR¹⁰; —NR¹¹R¹², or —CONR¹¹R¹²;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, oxo, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —NR¹⁴C₀₀R¹⁶, —(C₁-C₆ alkyl)- NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), —(C₁-C₆ alkyl)-SO₂(C₁-C₃ alkyl), optionally substituted carbocyclyl, optionally substituted heterocyclyl, optionally substituted aryl, or optionally substituted heteroaryl;
R⁵ and R⁶ are each independently hydrogen, halogen, —OH, optionally substituted C₁-C₃ alkyl, optionally substituted C₂-C₃ alkenyl, optionally substituted C₂-C₃ alkynyl, or C₁-C₃ alkoxy; or R⁵ and R⁶ taken together form an optionally substituted 3- to 6-membered carbocyclyl or heterocyclyl;
R⁷ is hydrogen, C₁-C₆ alkyl, C₂-C₆ alkenyl, or C₂-C₆ alkynyl;
R⁸ and R⁹ are each independently hydrogen, halogen, or C₁-C₃ alkyl;
R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{9a} taken together form an optionally substituted 3- to 6-membered carbocyclyl or heterocyclyl;
R¹⁰ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl;
R¹¹ and R¹² are each independently hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl; or R¹¹ and R¹² taken together form an optionally substituted heterocyclyl which optionally contains one or two additional heteroatoms selected from N, S, or O;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form an optionally substituted 3- to 6-membered heterocyclyl;
R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;
each m is independently 0, 1, or 2;
n1 and n2 are each independently 0, 1, or 2;
n3 is 0, 1, 2, 3, 4 or 5; and
t is 0, 1 or 2.

In a specific embodiment of formula (I), (I-A), and/or (I-B), C is a heteroaryl ring.

In one embodiment the present disclosure provides compounds of formula (II):

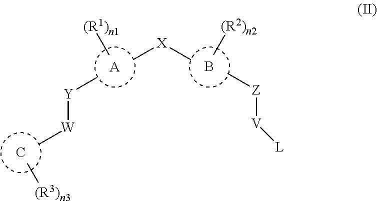

or a pharmaceutically acceptable salt, tautomer, stereoisomer or prodrug thereof, wherein:
A is a 4- to 15-membered ring selected from aryl, carbocyclyl, heteroaryl, or heterocyclyl;
B is a 4- to 15-membered ring selected from aryl, carbocyclyl, heteroaryl, or heterocyclyl;
C is a heteroaryl ring;
X is a bond, —(CR⁵R⁶)ₜ—, —C(═O)—, or —NR⁷;
Y is a bond, —(CR⁸R⁹)ₘ—, —O—, —S—, —S(═O)—, —SO₂—, —NR⁷—, or —N(COCH₃)—;
W is a bond, —(CR^{8a}R^{9a})ₘ—, —C(═O)—, —N(R⁷)CO—, —CONR⁷—, or —NSO₂R⁷—;
Z is a bond, —CH₂—, —C(CH₃)H—, —O—, —S—, —NH—, —NCH₃—, or —N(COCH₃)—;
V is —CH₂— or phenylene and L is halogen, —NH₂, —CHCl₂, —CCl₃, or —CF₃; or
V is —CH₂CH₂— and L is halogen or —NH₂;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, oxo, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —NR¹⁴C₀₀R¹⁶, —(C₁-C₆ alkyl)- NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is hydrogen, C₁-C₆ alkyl, C₂-C₆ alkenyl, or C₂-C₆ alkynyl;
R⁸ and R⁹ are each independently hydrogen, halogen, or C₁-C₃ alkyl;
R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{9a} taken together form an optionally substituted 3- to 6-membered carbocyclyl or heterocyclyl;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form an optionally substituted 3- to 6-membered heterocyclyl;
R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;
each m is independently 0, 1, or 2;
n1 and n2 are each independently 0, 1, or 2;
n3 is 1, 2, 3, 4 or 5; and
t is 0, 1 or 2.

In one embodiment of the compounds of formula (I), (I-A), (I-B), and/or (II), C is

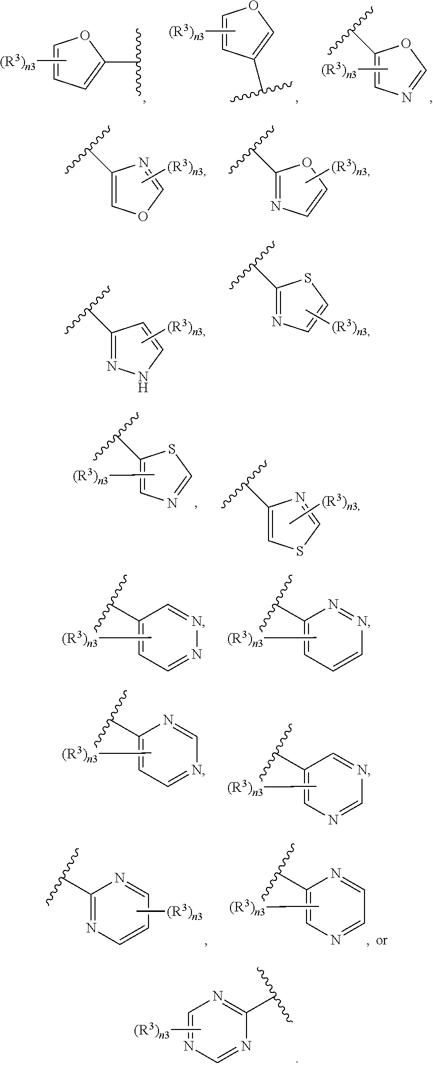

In one embodiment, the present disclosure provides compounds of formula (A):

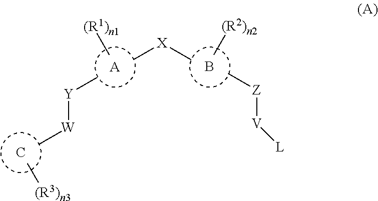

or a pharmaceutically acceptable salt, tautomer, stereoisomer or prodrug thereof, wherein:
A is a 5- to 15-membered ring selected from aryl, carbocyclyl, heteroaryl, or heterocyclyl;
B is a phenyl;
C is a heteroaryl ring;
X is a bond, —(CR⁵R⁶)ₜ—, or —C(═O)—;
Y is a bond, —(CR⁸R⁹)ₘ—, —O—, —S—, —S(═O)—, —SO₂—, —NR⁷—, or —N(COCH₃)—;
W is a bond, —(CR^{8a}R^{9a})ₘ—, —C(═O)—, —N(R⁷)CO—, —CONR⁷—, or —NSO₂R⁷—;
Z is a bond, —CH₂—, —C(CH₃)H—, —O—, —S—, —NH—, —NCH₃—, or —N(COCH₃)—;
V is —CH₂— and L is halogen, —NH₂, —CHCl₂, —CCl₃, or —CF₃; or
V is —CH₂CH₂— and L is halogen or —NH₂;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, oxo, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —NR¹⁴C₀₀R¹⁶, —(C₁-C₆ alkyl)- NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is hydrogen, C₁-C₆ alkyl, C₂-C₆ alkenyl, or C₂-C₆ alkynyl;
R⁸ and R⁹ are each independently hydrogen, halogen, or C₁-C₃ alkyl;
R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{9a} taken together form an optionally substituted 3- to 6-membered carbocyclyl or heterocyclyl;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form an optionally substituted 3- to 6-membered heterocyclyl;
R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;
each m is independently 0, 1, or 2;
n1 and n2 are each independently 0, 1, or 2;
n3 is 1, 2, 3, 4 or 5; and
t is 0, 1 or 2.

In one embodiment, the present disclosure provides compounds of formula (A-I):

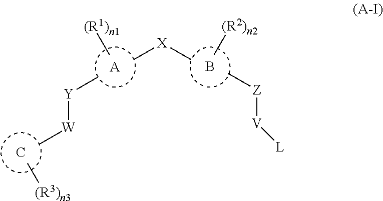

or a pharmaceutically acceptable salt, tautomer, stereoisomer or prodrug thereof, wherein:
A is a fused or a bridged bicyclic 5- to 15-membered ring selected from aryl, carbocyclyl, heteroaryl, or heterocyclyl;
B is a phenyl;
C is a heteroaryl ring;
X is a bond, —CH₂—, —C(CH₃)₂—, or —C(═O)—;
Y is a bond or —O—;
W is —CH₂— or —C(CH₃)H—;
Z is a bond or —O—;
V is —CH₂— and L is halogen, —CHCl₂, —CCl₃, or —CF₃; or
V is —CH₂CH₂— and L is halogen;
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —NHCOO(C₁-C₃ alkyl), —N(CH₃)CO(C₁-C₃ alkyl), or —N(CH₃)COO(C₁-C₃ alkyl);
n1 and n2 are each independently 0, 1, or 2; and
n3 is 1, 2, 3, 4 or 5.

In one embodiment of the compounds of formula (A) or (A-I):
X is bond or —C(CH₃)₂—;
Y and Z are each —O—;
V is —CH₂— or —CH₂CH₂—;
L is halogen;
R¹ and R² are each independently hydrogen, halogen, —CN, or —CF₃; and
R¹⁶ is hydrogen, C₁-C₃ alkyl, C₂-C₃ alkenyl, or C₂-C₃ alkynyl.

In one embodiment of the compounds of formula (A) or (A-I):
W is —CH₂— or —C(CH₃)H—;
V is —CH₂CH₂—; and
R¹ and R² are each independently hydrogen, halogen, —CN, or —CF₃.

In one embodiment, the present disclosure provides compounds of formula (A-II):

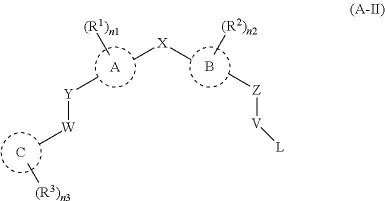

or a pharmaceutically acceptable salt, tautomer, stereoisomer or prodrug thereof, wherein:
A is a fused or a bridged bicyclic 5- to 15-membered ring selected from aryl, carbocyclyl, heteroaryl, or heterocyclyl;
B is a phenyl or a 6-membered heteroaryl or heterocyclyl;
C is a 3- to 10-membered aryl, carbocyclyl, heteroaryl, or heterocyclyl;
X is a bond, —CH₂—, —C(CH₃)₂—, or —C(═O)—;
Y is a bond, —CH₂—, —NH—, or —O—;
W is a bond, —CH₂—, —CH₂CH₂—, —C(CH₃)H—, —N(R⁷)CO—, or —CONR⁷—;
Z is a bond or —O—;
V is a bond, —CH₂(CR^{8a′}R^{9a′})—, or —(CR^{8a}R^{9a})ₘ—;
L is hydrogen, halogen, —CF₂R¹⁰, —CF₃, —CN, —OR¹⁰, —NR¹¹R¹², or —CONR¹¹R¹²;
R¹ and R² are each independently hydrogen, halogen, oxo, —CN, or —CF₃;
at least one R³ is —CH₂NHSO₂CH₃, —CH₂N(CH₃)SO₂CH₃, —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), or —N(CH₃)COO(C₁-C₃ alkyl);
R⁷ is hydrogen or C₁-C₃ alkyl;
R^{8a} and R^{9a} are each independently hydrogen, halogen, or C₁-C₃ alkyl;
R^{8a′} and R^{9a′} taken together form an optionally substituted 3- to 6-membered carbocyclyl or heterocyclyl containing one, two, or three heteroatoms selected from N, S, or O;
R¹⁰ is hydrogen, or C₁-C₃ alkyl;
R¹¹ and R¹² are each independently hydrogen or C₁-C₃ alkyl; or R¹¹ and R¹² taken together form an optionally substituted heterocyclyl which optionally contains one or two additional heteroatoms selected from N, S, or O;
m is 0, 1, or 2;
n1 and n2 are each independently 0, 1, or 2; and
n3 is 1, 2, 3, 4 or 5.

In one embodiment, the present disclosure provides compounds of formula (A-III):

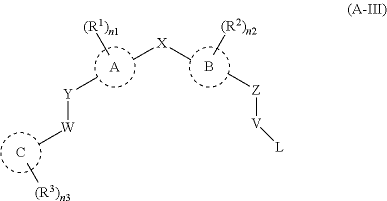

or a pharmaceutically acceptable salt, tautomer, stereoisomer or prodrug thereof, wherein:
A is a fused or a bridged bicyclic 5- to 15-membered ring selected from aryl, carbocyclyl, heteroaryl, or heterocyclyl;
B is a phenyl or a 6-membered heteroaryl or heterocyclyl;
C is a 4- to 10-membered aryl, carbocyclyl, heteroaryl, or heterocyclyl;
X is a bond, —CH₂—, —C(CH₃)₂—, or —C(═O)—;
Y is a bond, —CH₂—, —C(CH₃)H—, —NH—, or —O—;
W is —CH₂—, —CH₂CH₂—, —C(CH₃)H—, —NR⁷—, —N(R⁷)CO—, or —CONR⁷—;
Z is a bond or —O—;
V is a bond, —CH₂(CR^{8a′}R^{9a′})—, —CR^{8a}═CR^{9a}—, or —(CR^{8a}R^{9a})ₘ—;
L is hydrogen, halogen, —CF₂R¹⁰, —CF₃, —CN, —OR¹⁰, —NR¹¹R¹², —SO₂R¹⁷, or —CONR¹¹R¹²;
R¹ and R² are each independently hydrogen, halogen, oxo, —CN, or —CF₃;
at least one R³ is —CH₂NHSO₂CH₃, —CH₂N(CH₃)SO₂CH₃, —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), or —N(CH₃)COO(C₁-C₃ alkyl);
R⁷ is hydrogen or C₁-C₃ alkyl;
R^{8a} and R^{9a} are each independently hydrogen, halogen, or C₁-C₃ alkyl;
R^{8a′} and R^{9a′} taken together form an optionally substituted 3- to 6-membered carbocyclyl or heterocyclyl containing one, two, or three heteroatoms selected from N, S, or O;
R¹⁰ is hydrogen, or C₁-C₃ alkyl;
R¹¹ and R¹² are each independently hydrogen or C₁-C₃ alkyl; or R¹¹ and R¹² taken together form an optionally substituted heterocyclyl which optionally contains one or two additional heteroatoms selected from N, S, or O;
R¹⁷ is hydrogen or C₁-C₃ alkyl;
m is 0, 1, or 2;
n1 and n2 are each independently 0, 1, or 2; and
n3 is 1, 2, 3, 4 or 5.

In one embodiment of the compounds of formula (I), (I-A), (I-B), (II), and/or (A), A is a fused or a bridged bicyclic ring.

In one embodiment of the compounds of formula (I), (II), (A), (A-I), (A-II), and/or (A-III), A is a 6,6-fused ring, 6,5-fused ring, or 5,6-fused ring, each optionally substituted with one or two R¹.

In one embodiment of the compounds of formula (I), (II), (A), (A-I), (A-II), and/or (A-III), A is

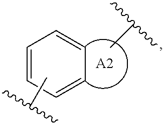

optionally substituted with one or two R¹, wherein A2 is a 5- or a 6-membered aryl, carbocyclyl, heteroaryl, or heterocyclyl ring. In one embodiment, A2 is a 6-membered aryl, carbocyclyl, heteroaryl or heterocyclyl ring. In one embodiment, A2 is a 6-membered aryl or heteroaryl ring. In one embodiment, A2 is a 5-membered carbocyclyl, heteroaryl or heterocyclyl ring. In one embodiment, A2 is a 5-membered heterocyclyl or heteroaryl ring. In one embodiment, heterocyclyl ring contains one, two, or three heteroatoms selected from N, S, or O. In one embodiment, heteroaryl contains one, two, or three heteroatoms selected from N, S, or O. In one embodiment, heteroaryl contains one or two nitrogen atoms as ring atoms.

In one embodiment of the compounds of formula (I), (II), (A), (A-I), (A-II), and/or (A-III), A is

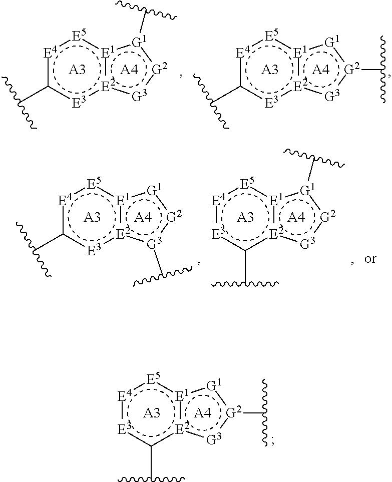

wherein:
ring A3 is aromatic;
E¹, E², E³, E⁴, and E⁵ are each independently, C, CR¹ or N;
G¹, G², and G³, are each independently, C, CR¹, C(R¹)₂, O, S, N, or NR¹;
wherein at least two of E¹, E², E³, E⁴, and E⁵ is C or CR¹ (i.e., maximum 3 of E¹, E², E³, E⁴, and E⁵ can be N); and
wherein at least two of E¹, E², G¹, G², and G³, is C, CR¹ or C(R¹)₂ (i.e., maximum 3 of E¹, E², G¹, G², and G³ can be heteroatoms).

In one embodiment, ring A3 is phenyl. In one embodiment, ring A3 is a heteroaryl ring. In one embodiment, ring A3 is a pyridine, pyridazine, pyrimidine, pyrazine, 1,2,4-trizaine, or 1,3,5-triazine ring.

In one embodiment, ring A4 is aromatic. In one embodiment, ring A4 is a heteroaryl ring.

In one embodiment, ring A4 is partially aromatic. In one embodiment, ring A4 is a heterocyclyl or a carbocyclyl ring. In one embodiment, the bond between E¹-G¹, G¹-G², G²-G³, and G³-E² are each a single bond.

In one embodiment of the compounds of formula (I), (II), (A), (A-I), (A-II), and/or (A-III), A is

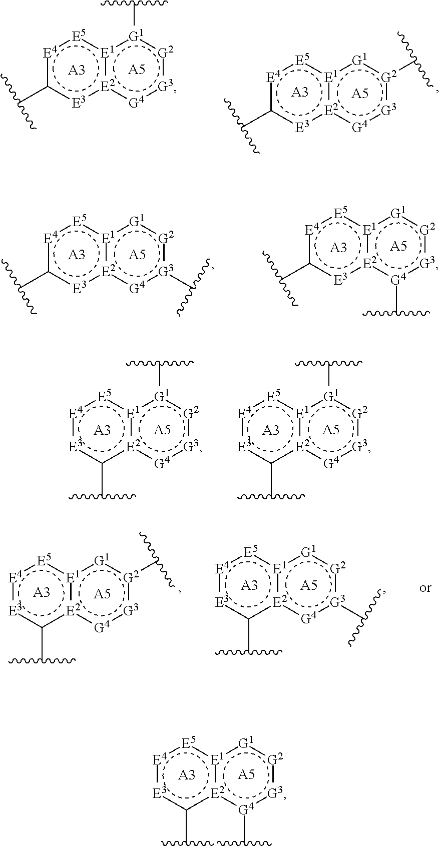

wherein:
ring A3 is aromatic;
E¹, E², E³, E⁴, and E⁵ are each independently, C, CR¹ or N;
G¹, G², G³, and G⁴, are each independently, C, CR¹, C(R¹)₂, O, S, N, or NR¹;
wherein at least two of E¹, E², E³, E⁴, and E⁵ is C or CR¹ (i.e., maximum 3 of E¹, E², E³, E⁴, and E⁵ can be N); and
wherein at least three of E¹, E², G¹, G², G³, and G⁴, is C, CR¹ or C(R¹)₂ (i.e., maximum 3 of E¹, E², G¹, G², G³, and G⁴ can be heteroatoms).

In one embodiment, ring A3 is phenyl. In one embodiment, ring A3 is a heteroaryl ring. In one embodiment, ring A3 is a pyridine, pyridazine, pyrimidine, pyrazine, 1,2,4-trizaine, or 1,3,5-triazine ring.

In one embodiment, E¹, E², E³, E⁴, and E⁵ is N, C, CH or CR¹. In one embodiment, E1 and E² are C, and E³, E⁴, and E⁵ are each independently N, CH or CR¹.

In one embodiment, ring A5 is aromatic. In one embodiment, ring A5 is a heteroaryl ring.

In one embodiment, ring A5 is partially aromatic. In one embodiment, ring A5 is a heterocyclyl or a carbocyclyl ring. In one embodiment, the bond between E¹-G¹, G¹-G², G²-G³, G³-G⁴ and G³-E² are each a single bond. In one embodiment, one of the bond between E1-G¹, G¹-G², G²-G³, G³-G⁴ and G³-E² is a double bond.

In one embodiment, E¹ and E² are each independently N or C, and G¹, G², G³, and G⁴, are each independently N, NR¹, C, CH, CR¹, O, or S.

In one embodiment of the compounds of formula (I), (II), (A), (A-I), (A-II), and/or (A-III), A is a ring selected from bicyclo[1.1.1]pentane, 4,5,6,7-tetrahydroindole, indoline, indole, pyrazolo[1,5-a]pyridine, imidazo[1,2-a]pyridine, indazole, benzo[d]imidazole, benzo[d]isoxazole, benzo[b]thiophene, 1,3-dihydroisobenzofuran, quinazoline, 3,4-dihydrobenzo[b][1,4]oxazine, benzo[d][1,2,3]triazole, naphthalene, 1,2-dihydronaphthalene, 1,2,3,4-tetrahydronaphthalene, 2,3-dihydroindene, 2,3-dihydrobenzo[b][1,4]dioxine, isoindoline, or isoindolin-1-one, each ring is optionally substituted with one or two R¹. In one embodiment, A is a 5- to 10-membered fused or a bridged bicyclic ring. In one embodiment, A is ring selected from bicyclo[1.1.1]pentane, indoline, indole, indazole, quinazoline, 3,4-dihydro-2H-benzo[b][1,4]oxazine, 1H-benzo[d][1,2,3]triazole, naphthalene, 1,2-dihydronaphthalene, 1,2,3,4-tetrahydronaphthalene, 2,3-dihydro-1H-indene, 2,3-dihydrobenzo[b][1,4]dioxine, isoindoline, or isoindolin-1-one, each ring is optionally substituted with one or two R¹.

In one embodiment of the compounds of formula (I), (II), (A), (A-I), (A-II), and/or (A-III), A is

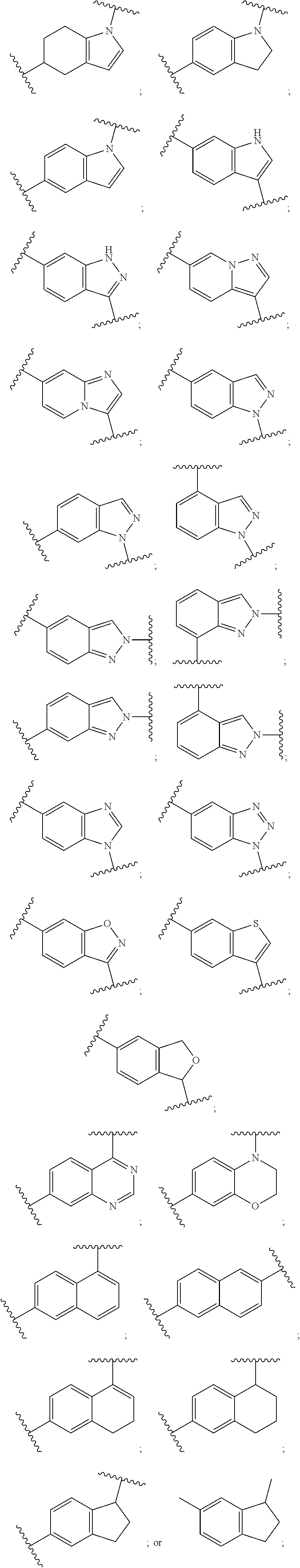

wherein each ring is optionally substituted with one or two R¹. In one embodiment, A is

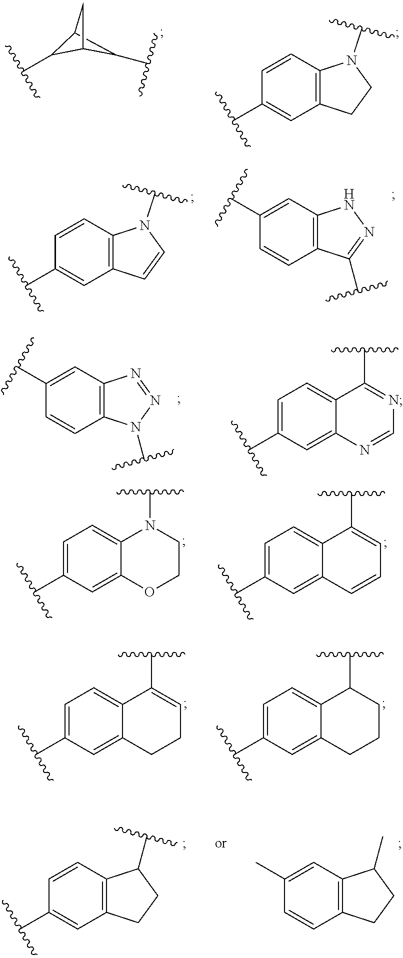

wherein each ring is optionally substituted with one or two R¹.

In one embodiment of the compounds of formula (A), (A-I), (A-II), and/or (A-III), C is

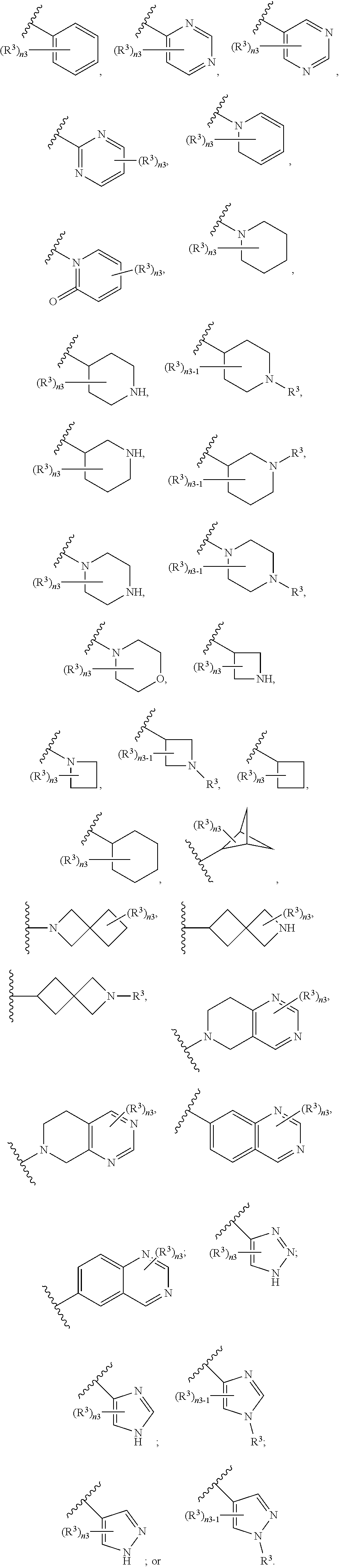

In one embodiment of the compounds of formula (A), (A-I), (A-II), and/or (A-III):
X is bond or —C(CH₃)₂—;
Y and Z are each —O—;
V is —CH₂— or —CH₂CH₂—;
L is halogen;
R¹ and R² are each independently hydrogen, halogen, —CN, or —CF₃; and
R¹⁶ is hydrogen, C₁-C₃ alkyl, C₂-C₃ alkenyl, or C₂-C₃ alkynyl.

In one embodiment of the compounds of formula (A), (A-I), (A-II), and/or (A-III):
W is —CH₂— or —C(CH₃)H—;
V is —CH₂CH₂—; and
R¹ and R² are each independently hydrogen, halogen, —CN, or —CF₃.

In one embodiment of the compounds of formula (A-III):
Y is —CH₂— or —C(CH₃)H—; and
W is —NH—.

In one embodiment of the compounds of formula (A-III), W is —NH—.

In one embodiment of the compounds of formula (A-III):
Y is —CH₂— or —C(CH₃)H—;
W is —NH—;
X is bond or —C(CH₃)₂—;
Z is —O—;
V is —CH₂— or —CH₂CH₂—;
L is halogen; and
R¹ and R² are each independently hydrogen, halogen, —CN, or —CF₃.

In one embodiment of the compounds of formula (A), (A-I), (A-II), and/or (A-III):
A is a 6,6-fused ring, 6,5-fused ring, or 5,6-fused ring, each optionally substituted with one or two R¹;
X is bond;
Y and Z are each —O—;
V is —CH₂— or —CH₂CH₂—;
L is halogen;
R¹ and R² are each independently hydrogen, halogen, —CN, or —CF₃; and
R¹⁶ is hydrogen, C₁-C₃ alkyl, C₂-C₃ alkenyl, or C₂-C₃ alkynyl.

In one embodiment of the compounds of formula (A), (A-I), (A-II), and/or (A-III):
A is a 6,6-fused ring, 6,5-fused ring, or 5,6-fused ring, each optionally substituted with one or two R¹;
X is bond;
Y and Z are each —O—;
W is —CH₂— or —C(CH₃)H—;
V is —CH₂CH₂—;
L is halogen;
R¹ and R² are each independently hydrogen, halogen, —CN, or —CF₃; and
R¹⁶ is hydrogen, C₁-C₃ alkyl, C₂-C₃ alkenyl, or C₂-C₃ alkynyl.

In one embodiment, the present disclosure provides compounds of formula (B):

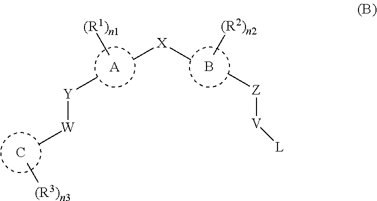

or a pharmaceutically acceptable salt, tautomer, stereoisomer or prodrug thereof, wherein:
A is a phenyl;
B is a 5- to 15-membered ring selected from aryl, carbocyclyl, heteroaryl, or heterocyclyl;
C is a heteroaryl ring;
X is a bond, —(CR⁵R⁶)ₜ—, or —C(═O)—;
Y is a bond, —(CR⁸R⁹)ₘ—, —O—, —S—, —S(═O)—, —SO₂—, —NR⁷—, or —N(COCH₃)—;
W is a bond, —(CR^{8a}R^{9a})ₘ—, —C(═O)—, —N(R⁷)CO—, —CONR⁷—, or —NSO₂R⁷—;
Z is a bond, —CH₂—, —C(CH₃)H—, —O—, —S—, —NH—, —NCH₃—, or —N(COCH₃)—;
V is absent or —CH₂— and L is halogen, —NH₂, —CHCl₂, —CCl₃, or —CF₃; or
V is —CH₂CH₂— and L is halogen or —NH₂;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, oxo, —NR¹³R¹⁴, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, —SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —NR¹⁴COOR¹⁶, —(C₁-C₆ alkyl)-NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO₂(C₁-C₃ alkyl), or —(C₁-C₆ alkyl)-SO₂(C₁-C₃ alkyl);
R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl;
R⁷ is hydrogen, C₁-C₆ alkyl, C₂-C₆ alkenyl, or C₂-C₆ alkynyl;
R⁸ and R⁹ are each independently hydrogen, halogen, or C₁-C₃ alkyl;
R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{9a} taken together form an optionally substituted 3- to 6-membered carbocyclyl or heterocyclyl;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form an optionally substituted 3- to 6-membered heterocyclyl;
R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;
each m is independently 0, 1, or 2;
n1 and n2 are each independently 0, 1, or 2;
n3 is 1, 2, 3, 4 or 5; and
t is 0, 1 or 2.

In one embodiment, the present disclosure provides compounds of formula (B-I):

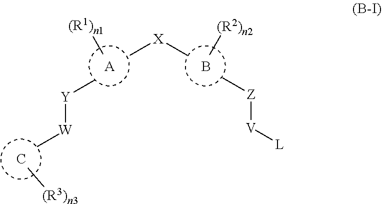

or a pharmaceutically acceptable salt, tautomer, stereoisomer or prodrug thereof, wherein:
A is a phenyl;
B is a fused or a bridged bicyclic 5- to 15-membered ring selected from aryl, carbocyclyl, heteroaryl, or heterocyclyl;
C is a heteroaryl ring;
X is a bond, —CH₂—, —C(CH₃)₂—, or —C(═O)—;
Y is a bond or —O—;
W is —CH₂— or —C(CH₃)H—;
Z is a bond or —O—;
V is absent or —CH₂— and L is halogen, —CHCl₂, —CCl₃, or —CF₃; or
V is —CH₂CH₂— and L is halogen;
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —NHCOO(C₁-C₃ alkyl), —N(CH₃)CO(C₁-C₃ alkyl), or —N(CH₃)COO(C₁-C₃ alkyl);
n1 and n2 are each independently 0, 1, or 2; and
n3 is 1, 2, 3, 4 or 5.

In one embodiment of the compounds of formula (B) or (B-I):
X is bond or —C(CH₃)₂—;
Y is —O—;
Z is a bond or —O—;
V is bond, —CH₂— or —CH₂CH₂—;
L is halogen;
R¹ and R² are each independently hydrogen, halogen, —CN, or —CF₃; and
R¹⁶ is hydrogen, C₁-C₃ alkyl, C₂-C₃ alkenyl, or C₂-C₃ alkynyl.

In one embodiment of the compounds of formula (B) or (B-I):
Y and Z are each —O—;
W is —CH₂— or —C(CH₃)H—;
V is —CH₂CH₂—; and
R¹ and R² are each independently hydrogen, halogen, —CN, or —CF₃.

In one embodiment of the compounds of formula (I), (I-A), (I-B), (II), (B) and/or (B-I), B is a fused or a bridged bicyclic ring, optionally substituted with one or two R².

In one embodiment of the compounds of formula (I), (I-A), (I-B), (II), (B) and/or (B-I), B is a 6,6-fused ring, 6,5-fused ring, or 5,6-fused ring, each optionally substituted with one or two R².

In one embodiment of the compounds of formula (I), (I-A), (I-B), (II), (B) and/or (B-I), B is

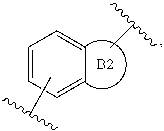

optionally substituted with one or two R², wherein B2 is a 5- or a 6-membered aryl, carbocyclyl, heteroaryl, or heterocyclyl ring. In one embodiment, B2 is a 6-membered aryl, carbocyclyl, heteroaryl or heterocyclyl ring. In one embodiment, B2 is a 6-membered aryl or heteroaryl ring. In one embodiment, B2 is a 5-membered carbocyclyl, heteroaryl or heterocyclyl ring. In one embodiment, B2 is a 5-membered heterocyclyl or heteroaryl ring. In one embodiment, heterocyclyl ring contains one, two, or three heteroatoms selected from N, S, or O. In one embodiment, heteroaryl contains one, two, or three heteroatoms selected from N, S, or O. In one embodiment, heteroaryl contains one or two nitrogen atoms as ring atoms.

In one embodiment of the compounds of formula (I), (I-A), (I-B), (II), (B) and/or (B-I), B is

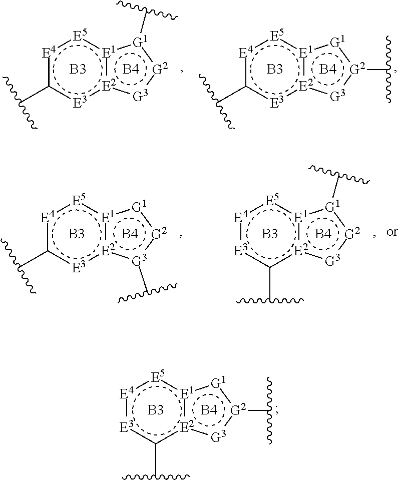

wherein:
ring B3 is aromatic;
E¹, E², E³, E⁴, and E⁵ are each independently, C, CR² or N;
G¹, G2, and G3, are each independently, C, CR², C(R²)₂, O, S, N, or NR²;
wherein at least two of E¹, E², E³, E⁴, and E⁵ is C or CR² (i.e., maximum 3 of E¹, E², E³, E⁴, and E⁵ can be N); and
wherein at least two of E¹, E², G¹, G², and G³, is C, CR² or C(R²)₂ (i.e., maximum 3 of E¹, E², G¹, G², and G³ can be heteroatoms).

In one embodiment, ring B3 is phenyl. In one embodiment, ring B3 is a heteroaryl ring. In one embodiment, ring B3 is a pyridine, pyridazine, pyrimidine, pyrazine, 1,2,4-trizaine, or 1,3,5-triazine ring.

In one embodiment, ring B4 is aromatic. In one embodiment, ring B4 is a heteroaryl ring.

In one embodiment, ring B4 is partially aromatic. In one embodiment, ring B4 is a heterocyclyl or a carbocyclyl ring. In one embodiment, the bond between E¹-G¹, G¹-G², G²-G³, and G³-E² are each a single bond.

In one embodiment of the compounds of formula (I), (I-A), (I-B), (II), (B) and/or (B-I), B is

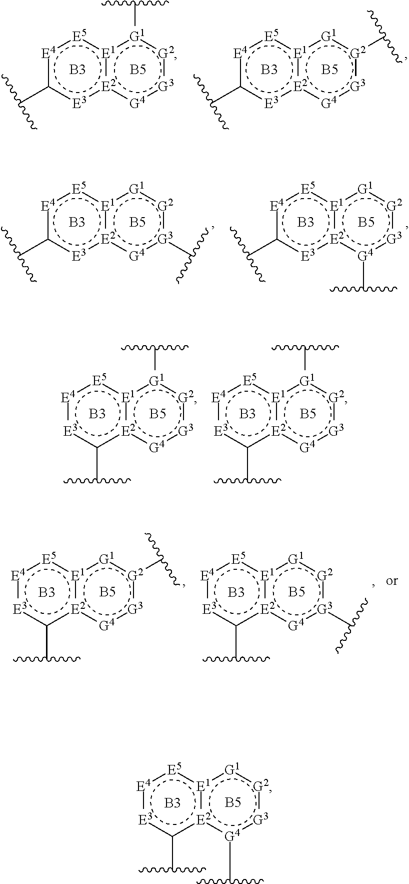

wherein:
ring B3 is aromatic;
E¹, E², E³, E⁴, and E⁵ are each independently, C, CR² or N;
G¹, G², G³, and G⁴, are each independently, C, CR², C(R²)₂, O, S, N, or NR²;
wherein at least two of E¹, E², E³, E⁴, and E⁵ is C or CR² (i.e., maximum 3 of E¹, E², E³, E⁴, and E⁵ can be N); and
wherein at least three of E¹, E², G¹, G², G³, and G⁴, is C, CR² or C(R²)₂ (i.e., maximum 3 of E¹, E², G¹, G², G³, and G⁴ can be heteroatoms).

In one embodiment, ring B3 is phenyl. In one embodiment, ring B3 is a heteroaryl ring. In one embodiment, ring B3 is a pyridine, pyridazine, pyrimidine, pyrazine, 1,2,4-trizaine, or 1,3,5-triazine ring.

In one embodiment, ring B5 is aromatic. In one embodiment, ring B5 is a heteroaryl ring.

In one embodiment, E¹, E², E³, E⁴, and E⁵ are each independently N, C, CH or CR². In one embodiment, E¹ and E² are C, and E³, E⁴, and E⁵ are each independently N, CH or CR².

In one embodiment, ring B5 is partially aromatic. In one embodiment, ring B5 is a heterocyclyl or a carbocyclyl ring. In one embodiment, the bond between E¹-G¹, G¹-G², G²-G³, G³-G⁴ and G³-E² are each a single bond. In one embodiment, one of the bonds between E¹-G¹, G¹-G², G²-G³, G³-G⁴ and G³-E² is a double bond.

In one embodiment, E¹ and E² are each independently N or C, and G¹, G², G³, and G⁴, are each independently N, NR¹, C, CH, CR¹, O, or S.

In one embodiment of the compounds of formula (I), (I-A), (I-B), (II), (B) and/or (B-I), B is a 5- to 10-membered fused or a bridged bicyclic ring, optionally substituted with one or two R². In one embodiment, B is an 8- to 10-membered fused or a bridged bicyclic ring, optionally substituted with one or two R². In one embodiment, B is ring selected from bicyclo[1.1.1]pentane, indoline, indole, indazole, quinazoline, 3,4-dihydrobenzo[b][1,4]oxazine, benzo[d][1,2,3]triazole, naphthalene, 1,2-dihydronaphthalene, 1,2,3,4-tetrahydronaphthalene, 2,3-dihydroindene, 2,3-dihydrobenzo[b][1,4]dioxine, isoindoline, or isoindolin-1-one, each ring is optionally substituted with one or two R². In one embodiment, B is ring selected from bicyclo[1.1.1]pentane, indoline, indole, indazole, quinazoline, 3,4-dihydro-2H-benzo[b][1,4]oxazine, 1H-benzo[d][1,2,3]triazole, naphthalene, 1,2-dihydronaphthalene, 1,2,3,4-tetrahydronaphthalene, 2,3-dihydro-1H-indene, 2,3-dihydrobenzo[b][1,4]dioxine, isoindoline, or isoindolin-1-one, each ring is optionally substituted with one or two R². In one embodiment, B is:

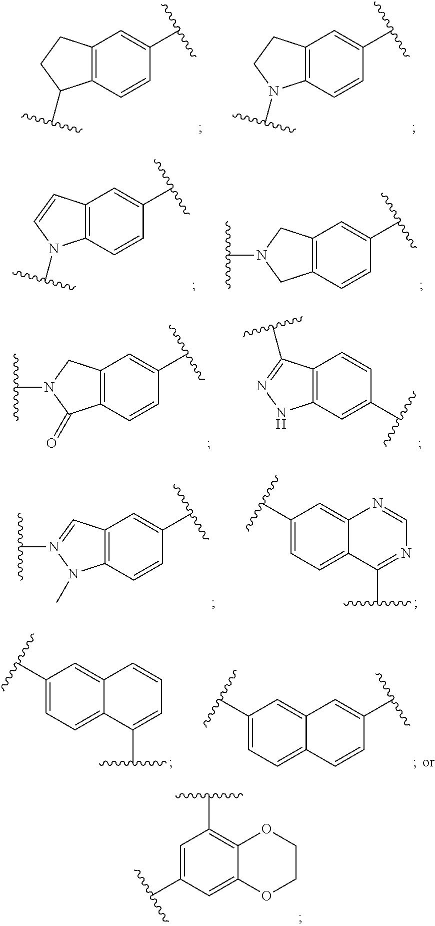

wherein each ring is optionally substituted with one or two R².

In one embodiment of the compounds of formula (B) and/or (B-I):
B is a 6,6-fused ring, 6,5-fused ring, or 5,6-fused ring, each optionally substituted with one or two R²;
X is bond;
Y is —O—;
Z is a bond or —O—;
V is bond, —CH₂— or —CH₂CH₂—;
L is halogen;
R¹ and R² are each independently hydrogen, halogen, —CN, or —CF₃; and
R¹⁶ is hydrogen, C₁-C₃ alkyl, C₂-C₃ alkenyl, or C₂-C₃ alkynyl.

In one embodiment of the compounds of formula (B) and/or (B-I):
B is a 6,6-fused ring, 6,5-fused ring, or 5,6-fused ring, each optionally substituted with one or two R²;
X is bond;
Y and Z are each —O—;
W is —CH₂— or —C(CH₃)H—;
V is —CH₂CH₂—;
L is halogen;
R¹ and R² are each independently hydrogen, halogen, —CN, or —CF₃; and
R¹⁶ is hydrogen, C₁-C₃ alkyl, C₂-C₃ alkenyl, or C₂-C₃ alkynyl.

In one embodiment of the compounds of formula (I), (I-A), (I-B), (II), (A), (A-I), (A-II), (A-III), (B), and/or (B-I), C is a pyrimidine, triazine, or thiophene ring.

In one embodiment of the compounds of formula (I), (I-A), (I-B), (II), (A), (A-I), (A-II), (A-III), (B), and/or (B-I), C is

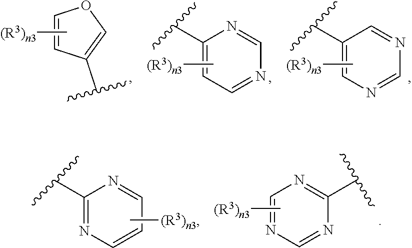

In one embodiment, C is

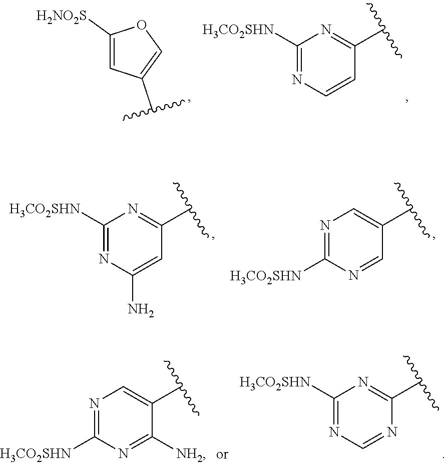

In one embodiment of the compounds of formula (I), (I-A), (I-B), (II), (A), (A-I), (A-II), (A-III), (B), and/or (B-I), C is a pyrimidine ring. In one embodiment, C is

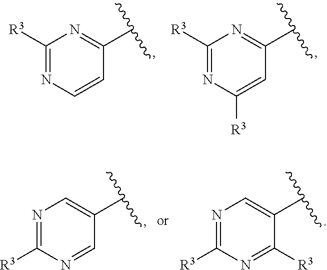

In one embodiment, C is

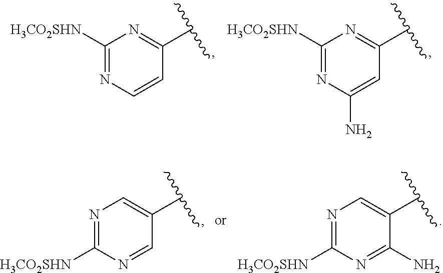

In one embodiment of the compounds of formula (I), (I-A), (I-B), (II), (A), (A-I), (A-II), (A-III), (B), and/or (B-I), R¹ and R² are each independently Cl, —CN, or —CF₃. In one embodiment, R¹ and R² are each independently C₁ or —CN.

In one embodiment of the compounds of formula (I), (I-A), (I-B), (II), (A), (A-I), (A-II), (A-III), (B), and/or (B-I), at least one R² is —CN. In one embodiment, at least one R² is —Cl. In one embodiment, n2 is at least 2; at least one R² is —CN; and at least one R² is —Cl.

In one embodiment of the compounds of formula (I), R³ is not hydrogen.

In one embodiment of the compounds of formula (I), (I-A), (I-B), (II), (A), and/or (B), R³ is —NR¹⁴SO₂R¹⁶, wherein R¹⁴ and R¹⁶ together form a 5 or 6 membered ring including the nitrogen and sulfur atoms.

In one embodiment of the compounds of formula (I), (I-A), (I-B), (II), (A), and/or (B), R³ is —NR¹⁴SO₂R¹⁶, wherein R¹⁶ is optionally substituted C₁-C₆ alkyl. In one embodiment, R³ is —NR¹⁴SO₂R¹⁶, wherein R¹⁶ is C₁-C₆ alkyl optionally substituted with one or more groups selected from halogen, —CN, —CF₃, —OH, C₁-C₃ alkyl, C₁-C₃ alkoxy, —NH₂, —NH(C₁-C₃ alkyl), —N(C₁-C₃ alkyl)₂, —SCH₃. In one embodiment, R³ is —NR¹⁴SO₂R¹⁶, wherein R¹⁶ is C₁-C₃ alkyl substituted with —NH₂.

In one embodiment of the compounds of formula (I), (I-A), (I-B), (II), (A), (A-I), (A-II), (A-III), (B), and/or (B-I), R³ is F, Cl, Br, I, —CN, —CF₃, —OH, methyl, methoxy, —S(C₁-C₃ alkyl), —SO₂(C₁-C₃ alkyl), —NH₂, —NHSO₂CH₃, —NHSO₂CF₃, —N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), or —NHCO(C₁-C₃ alkyl).

embodiment of the compounds of formula (I), (I-A), (I-B), (II), (A), (A-I), (A-II), (A-III), (B), and/or (B-I), at least one R³ is —NHSO₂CH₃, —NHSO₂CH₂CH₃, —SO₂NH₂, or —SO₂CH₃. In one embodiment, if more than one R³ is present, the other R³ is —CN, C₁-C₃ alkyl, C₁-C₃ alkoxy, —SO₂(C₁-C₃ alkyl), —NH₂, —(C₁-C₃ alkyl)NH₂, —NHSO₂CH₃, —N(CH₃)SO₂CH₃, —NHSO₂CH₂CH₃, —N(CH₃)SO₂CH₂CH₃, —SO₂NH₂, —CONH₂, —CON(C₁-C₃ alkyl)₂, —CONH(C₁-C₃ alkyl), —NHCO(C₁-C₃ alkyl), —NHCOO(C₁-C₃ alkyl), —N(CH₃)CO(C₁-C₃ alkyl), or —N(CH₃)COO(C₁-C₃ alkyl).

In one embodiment of the compounds of formula (I), (I-A), (I-B), (II), (A), (A-II), (A-III), (B), and/or (B-I), at least one R³ is —CH₂NHSO₂CH₃, —CH₂N(CH₃)SO₂CH₃, —NHSO₂CH₃, —NHSO₂CH₂CH₃, —SO₂NH₂, or —SO₂CH₃. In one embodiment, if more than one R³ is present, the other R³ is —CN, C₁-C₃ alkyl, C₁-C₃ alkoxy, —SO₂(C₁-C₃ alkyl), —NH₂, —(C₁-C₃ alkyl)NH₂, —NHSO₂CH₃, —N(CH₃)SO₂CH₃, —NHSO₂CH₂CH₃, —N(CH₃)SO₂CH₂CH₃, —SO₂NH₂, —CONH₂, —CON(C₁-C₃ alkyl)₂, —CONH(C₁-C₃ alkyl), —NHCO(C₁-C₃ alkyl), —NHCOO(C₁-C₃ alkyl), —N(CH₃)CO(C₁-C₃ alkyl), or —N(CH₃)COO(C₁-C₃ alkyl).

In one embodiment of the compounds of formula (I), (I-A), (I-B), (II), (A), (A-II), (A-III), and/or (B), at least one R³ is —CH₂NHSO₂CH₃, —CH₂N(CH₃)SO₂CH₃, —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), or —N(CH₃)COO(C₁-C₃ alkyl).

In one embodiment of the compounds of formula (I), (I-A), (I-B), (II), (A), (A-I), (A-II), (A-III), (B), and (B-I), R³ is —NHSO₂CH₃, —NHSO₂CH₂CH₃, —SO₂NH₂, or —SO₂CH₃. In one embodiment, R³ is —NHSO₂CH₃, —NHSO₂CH₂CH₃, —SO₂NH₂, or —SO₂CH₃ and the other R³ is, if present (e.g., when n3 is 2, 3, or 5), —CN, C₁-C₃ alkyl, C₁-C₃ alkoxy, —SO₂(C₁-C₃ alkyl), —NH₂, —(C₁-C₃ alkyl)NH₂, —NHSO₂CH₃, —N(CH₃)SO₂CH₃, —NHSO₂CH₂CH₃, —N(CH₃)SO₂CH₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl).

In one embodiment of the compounds of formula (I), (I-A), (I-B), (II), (A), (A-I), (A-II), (A-III), (B), and/or (B-I), R³ is —NHSO₂CH₃, —NHSO₂CH₂CH₃, —SO₂NH₂, —SO₂CH₃, or —NH₂. In one embodiment of the compounds of formula (I), (I-A), (I-B), (II), (A), (A-I), (A-II), (A-III), (B), and (B-I), at least one R³ is —NHSO₂CH₃.

In one embodiment of the compounds of formula (I), (I-A), (I-B), (II), (A), (A-I), (A-II), (A-III), (B), and/or (B-I), X is a bond, —CH₂—, —C(CH₃)₂—, or —C(═O)—. In one embodiment, X is a bond.

In one embodiment of the compounds of formula (I), (I-A), (I-B), (II), (A), (A-I), (A-II), (A-III), (B), and/or (B-I), Y and Z are each —O—.

In one embodiment of the compounds of formula (I), (I-A), (I-B), (II), (A), (A-I), (A-II), (A-III), (B), and/or (B-I):
C is a 5- or 6-membered heteroaryl ring;
at least one R² is CN; and
at least one R³ is —NHSO₂CH₃, —NHSO₂CH₂CH₃, —SO₂NH₂, or —SO₂CH₃.

In embodiment of the compounds of formula (I), (I-A), (I-B), (II), (A), (A-I), (A-II), (A-III), (B), and/or (B-I):
C is a pyrimidine ring;
at least one R² is CN; and
at least one R³ is —NHSO₂CH₃, —NHSO₂CH₂CH₃, —SO₂NH₂, or —SO₂CH₃.

In one embodiment of the compounds of formula (I), (I-A), (I-B), (II), (A), (A-I), (A-II), (A-III), (B), and/or (B-I),
C is a pyrimidine ring;
at least one R² is CN; and
at least one R³ is —NHSO₂CH₃.

In one embodiment of the compounds of formula (I), (I-A), (I-B), (II), (A), (A-I), (A-II), (A-III), (B), and/or (B-I), —V-L is —CH₂CH₂Cl, —CH₂CH₂CH₂Cl, —CH₂CH₂NH₂, or —CH₂CH₂CH₂NH₂. In one embodiment, —V-L is —CH₂CH₂Cl or —CH₂CH₂CH₂Cl.

In one embodiment of the compounds of formula (I), (I-A), (I-B), (II), (A), (A-I), (A-II), (A-III), (B), and/or (B-I), —V-L is —CH₂CH₂Cl.

In one embodiment of the compounds of formula (I), (I-A), (I-B), (II), (A), (A-II), (A-III), (B), and/or (B-I), —Y—W— is a bond, —OCH₂—, —OCH₂CH₂—, —OCH(CH₃)—, —NH—, —NHCH₂—, —NHC(═O)—, or —C(═O)NH—.

In one embodiment of the compounds of formula (I), (I-A), (I-B), (II), (A), (A-I), (A-II), (A-III), (B), and/or (B-I), —Y—W— is —OCH₂— or —OCH(CH₃)—.

In one embodiment of the compounds of formula (I), (I-A), (I-B), (II), (A), (A-I), and/or (B), R⁵ and R⁶ are each independently hydrogen, halogen, —OH, C₁-C₃ alkyl, C₂-C₃ alkenyl, C₂-C₃ alkynyl, or C₁-C₃ alkoxy; or R⁵ and R⁶ taken together form an optionally substituted 3- to 6-membered carbocyclyl or heterocyclyl. In some embodiments, R⁵ and R⁶ are each independently hydrogen, halogen, —OH, optionally substituted C₁-C₆ alkyl, optionally substituted C₂-C₆ alkenyl, optionally substituted C₂-C₆ alkynyl. In one embodiment, R⁵ and R⁶ are hydrogen, halogen, —OH, or C₁-C₃ alkyl. In one embodiment, R⁵ and R⁶ are each independently hydrogen, F, —OH, or C₁-C₃ alkyl. In one embodiment, R⁵ and R⁶ are each independently, hydrogen, F, —OH, or methyl. In one embodiment, R⁵ and R⁶ are each H. In one embodiment, R⁵ and R⁶ are each methyl. In one embodiment of the compounds of formula (I), (II), (A), and/or (B), R⁵ and R⁶ are each H or methyl.

In one embodiment of the compounds of formula (I), (I-A), (I-B), (II), (A), and/or (B), R⁷ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In some embodiments, R⁷ is hydrogen, optionally substituted C₁-C₃ alkyl, optionally substituted C₂-C₃ alkenyl, optionally substituted C₂-C₃ alkynyl, optionally substituted carbocyclyl, optionally substituted heterocyclyl, optionally substituted aryl, or optionally substituted heteroaryl. In some embodiments, R⁷ is hydrogen, C₁-C₆ alkyl, C₂-C₆ alkenyl, C₂-C₆ alkynyl, carbocyclyl, heterocyclyl, aryl, or heteroaryl. In some embodiments, R⁷ is hydrogen, C₁-C₃ alkyl, C₂-C₃ alkenyl, C₂-C₃ alkynyl, carbocyclyl, heterocyclyl, aryl, or heteroaryl. In some embodiments, R⁷ is hydrogen, C₁-C₆ alkyl, C₂-C₆ alkenyl, or C₂-C₆ alkynyl. In some embodiments, R⁷ is hydrogen, C₁-C₃ alkyl, C₂-C₃ alkenyl, or C₂-C₃ alkynyl. In some embodiments, R⁷ is hydrogen or C₁-C₆ alkyl. In some embodiments, R⁷ is hydrogen or C₁-C₄ alkyl. In some embodiments of the compounds of formula (I), (II), (A), and/or (B), R⁷ is hydrogen or C₁-C₃ alkyl.

In one embodiment of the compounds of formula (I), (I-A), (I-B), (II), (A), (A-II), (A-III), and/or (B), R^{8a} and R^{9a} are each independently hydrogen, halogen, optionally substituted C₁-C₆ alkyl, optionally substituted C₂-C₆ alkenyl, optionally substituted C₂-C₆ alkynyl, optionally substituted C₁-C₆ alkoxy, —NR¹³R¹⁴, optionally substituted —(C₁-C₆ alkyl)-NR¹³R¹⁴, —NR¹⁴COR¹⁶, optionally substituted —(C₁-C₆ alkyl)-NR¹⁴COR¹⁶, —CONR¹⁴R¹⁵, optionally substituted —(C₁-C₆ alkyl)-CONR¹⁴R¹⁵, optionally substituted carbocyclyl, optionally substituted heterocyclyl, optionally substituted aryl, or optionally substituted heteroaryl, or R^{8a} and R^{9a} taken together form an optionally substituted carbocyclyl or optionally substituted heterocyclyl. In some embodiments, R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{9a} taken together form an optionally substituted 3- to 6-membered carbocyclyl or heterocyclyl. In one embodiment, R^{8a} and R^{9a} on the same carbon atom is taken together to form an optionally substituted 3- to 6-membered carbocyclyl or heterocyclyl. In one embodiment, R^{8a} and R^{9a} are each independently hydrogen, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵. In one embodiment of the compounds of formula (I), (II), (A), and/or (B), R^{8a} and R^{9a} are not —OH. In one embodiment, R^{8a} and R^{9a} are not —OH.

In one embodiment of the compounds of formula (I), R⁷ and R^{8a} taken together form an optionally substituted heterocyclyl. In one embodiment, R⁷ and R^{8a} taken together form an optionally substituted 3- to 7-membered heterocycle.

In one embodiment of the compounds of formula (I), (I-A), (I-B), (II), (A), and/or (B), R⁸ and R⁹ are each independently hydrogen, halogen, or C₁-C₃ alkyl.

In one embodiment of the compounds of formula (I), (I-A), (I-B), (II), (A), and/or (B), R¹³ and R¹⁴ are each independently hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In one embodiment, R¹³ and R¹⁴ are each independently hydrogen, optionally substituted C₁-C₆ alkyl, optionally substituted C₂-C₆ alkenyl, or optionally substituted C₂-C₆ alkynyl. In some embodiments R¹³ and R¹⁴ are each independently hydrogen, C₁-C₆ alkyl, C₂-C₆ alkenyl, or C₂-C₆ alkynyl. In some embodiments R¹³ and R¹⁴ are each independently hydrogen or C₁-C₃ alkyl.

In one embodiment of the compounds of formula (I), (I-A), (I-B), (II), (A), and/or (B), R¹⁵ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In one embodiment, R¹⁵ is hydrogen, optionally substituted C₁-C₆ alkyl, optionally substituted C₂-C₆ alkenyl, or optionally substituted C₂-C₆ alkynyl. In some embodiments, R¹⁵ is hydrogen, C₁-C₆ alkyl, C₂-C₆ alkenyl, or C₂-C₆ alkynyl. In some embodiments, R¹⁵ is hydrogen or C₁-C₃ alkyl.

In one embodiment of the compounds of formula (I), (I-A), (I-B), (II), (A), or (B), R¹⁴ and R¹⁵ taken together form an optionally substituted heterocyclyl. In one embodiment, R¹⁴ and R¹⁵ taken together form an optionally substituted 3- to 7-membered heterocyclyl. In other embodiments, R¹⁴ and R¹⁵ taken together form 3- to 7-membered heterocyclyl.

In one embodiment of the compounds of formula (I), (I-A), (I-B), (II), (A), and/or (B), R¹⁶ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl. In some embodiments, R¹⁶ is hydrogen, optionally substituted C₁-C₃ alkyl, optionally substituted C₂-C₃ alkenyl, optionally substituted C₂-C₃ alkynyl, optionally substituted carbocyclyl, optionally substituted heterocyclyl, optionally substituted aryl, or optionally substituted heteroaryl. In some embodiments, R¹⁶ is hydrogen, C₁-C₃ alkyl, C₂-C₃ alkenyl, C₂-C₃ alkynyl, carbocyclyl, heterocyclyl, aryl, or heteroaryl. In some embodiments, R¹⁶ is hydrogen, C₁-C₃ alkyl, C₂-C₃ alkenyl, or C₂-C₃ alkynyl. In some embodiments, R¹⁶ is hydrogen or C₁-C₃ alkyl.

In one embodiment of the compounds of formula (I), (I-A), (I-B), (II), (A), and/or (B), mis 1 or 2.

In one embodiment of the compounds of formula (I), (II), (A), (A-II), (A-III) and/or (B), t is 1 or 2. In one embodiment, t is 1.

In one embodiment, the present disclosure provides compounds of formula (C):

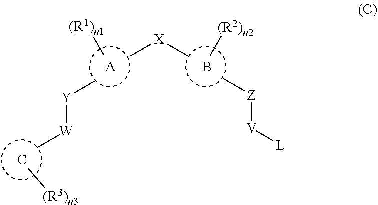

or a pharmaceutically acceptable salt, tautomer, stereoisomer or prodrug thereof, wherein:
A and B are each independently a phenyl ring;
C is a heteroaryl ring;
X is a bond, —CH₂—, —C(CH₃)₂—, or —C(═O)—;
Y is a bond or —O—;
W is —CH₂— or —C(CH₃)H—;
Z is a bond or —O—;
V is a phenylene;
L is halogen, —CHCl₂, —CCl₃, or —CF₃; or
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
n1 and n2 are each independently 0, 1, or 2; and
n3 is 1, 2, 3, 4 or 5.

In one embodiment, the present disclosure provides compounds of formula (E):

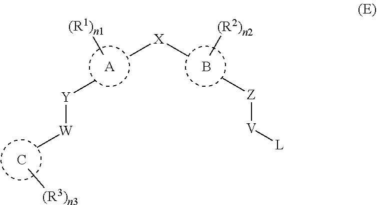

or a pharmaceutically acceptable salt, tautomer, stereoisomer or prodrug thereof, wherein:
A is a 5- or 6-membered heterocyclyl or heteroaryl;
B is phenyl;
C is a heteroaryl ring;
X is a bond, —CH₂—, —C(CH₃)₂—, or —C(═O)—;
Y is a bond or —O—;
W is —CH₂— or —C(CH₃)H—;
Z is a bond or —O—;
V is —CH₂— and L is halogen, —CHCl₂, —CCl₃, or —CF₃; or
V is —CH₂CH₂— and L is halogen;
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —NHCOO(C₁-C₃ alkyl), —N(CH₃)CO(C₁-C₃ alkyl), or —N(CH₃)COO(C₁-C₃ alkyl);
n1 and n2 are each independently 0, 1, or 2; and
n3 is 1, 2, 3, 4 or 5.

In one embodiment of the compounds of formula (E), the compound is not

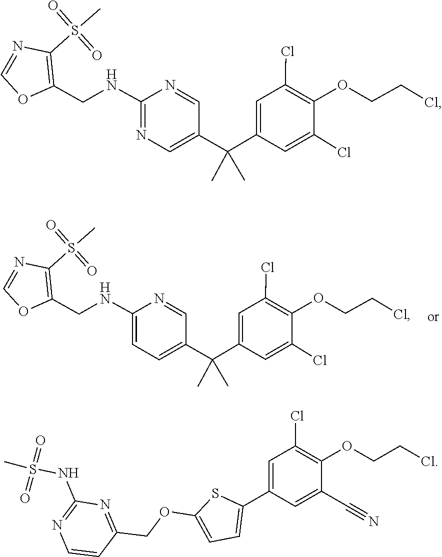 [SMILES: CC(C)(c1cnc(NCc2ocnc2S(C)(=O)=O)nc1)c1cc(Cl)c(OCCCl)c(Cl)c1 ; CC(C)(c1ccc(NCc2ocnc2S(C)(=O)=O)nc1)c1cc(Cl)c(OCCCl)c(Cl)c1 ; "or" ; CS(=O)(=O)Nc1nccc(COc2ccc(-c3cc(Cl)c(OCCCl)c(C#N)c3)s2)n1]

In one embodiment of the compounds of formula (E), C is a pyrimidine ring.

In one embodiment, the present disclosure provides compounds of formula (E-I):

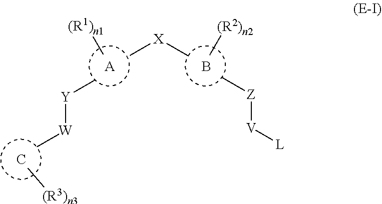

or a pharmaceutically acceptable salt, tautomer, stereoisomer or prodrug thereof, wherein:
A is a 5- or 6-membered heterocyclyl or heteroaryl;
B is phenyl;
C is a 6- to 12-membered heteroaryl ring;
X is a bond, —CH₂—, —CH(CH₃)—, —C(CH₃)₂—, or —C(═O)—;
Y is a bond, —O—, or —NH—;
W is a bond, —CH₂— or —C(CH₃)H—;
Z is a bond —NH—, or —O—;
V is —CH₂— and L is halogen, —CHCl₂, —CCl₃, or —CF₃; or
V is —CH₂CH₂— and L is halogen;
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —NHCOO(C₁-C₃ alkyl), —N(CH₃)CO(C₁-C₃ alkyl), or —N(CH₃)COO(C₁-C₃ alkyl);
n1 and n2 are each independently 0, 1, or 2; and
n3 is 1, 2, 3, or 4;
wherein A is not thiophene.

In one embodiment, the present disclosure provides compounds of formula (E-I), C is a fused bicyclic heteroaryl ring. In one embodiment, C is any fused bicyclic heteroaryl as described for formula (G).

In one embodiment, the present disclosure provides compounds of formula (E-I), C is

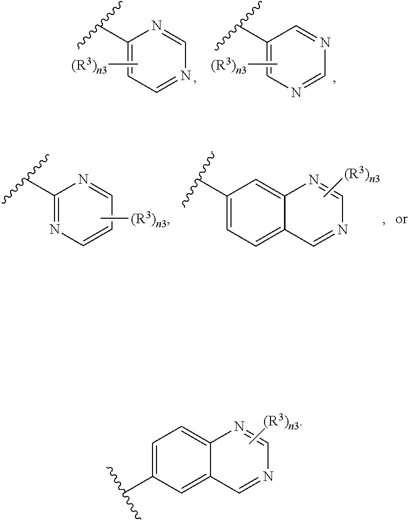

In one embodiment of the compounds of formula (E) and/or (E-I), A is piperidine, pyrazole, or oxadiazole ring, wherein each ring is optionally substituted with one or two R¹. In one embodiment, A is piperidine or oxadiazole ring, wherein each ring is optionally substituted with one or two R¹.

In one embodiment, the present disclosure provides compounds of formula (F):

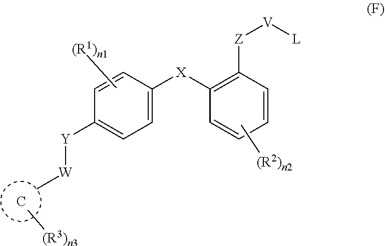

or a pharmaceutically acceptable salt, tautomer, stereoisomer or prodrug thereof, wherein:
A is a 5- or 6-membered heterocyclyl or heteroaryl;
B is phenyl;
C is a heteroaryl ring;
X is a bond, —CH₂—, —C(CH₃)₂—, or —C(═O)—;
Y is a bond or —O—;
W is —CH₂— or —C(CH₃)H—;
Z is a bond or —O—;
V is —CH₂— and L is halogen, —CHCl₂, —CCl₃, or —CF₃; or
V is —CH₂CH₂— and L is halogen;
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —NHCOO(C₁-C₃ alkyl), —N(CH₃)CO(C₁-C₃ alkyl), or —N(CH₃)COO(C₁-C₃ alkyl);
n1 and n2 are each independently 0, 1, or 2; and
n3 is 1, 2, 3, 4 or 5.

In one embodiment, the present disclosure provides compounds of formula (G):

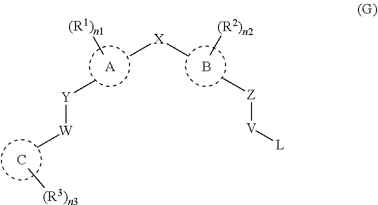

or a pharmaceutically acceptable salt, tautomer, stereoisomer or prodrug thereof, wherein:
A and B are each independently a phenyl ring;
C is a fused or a bridged bicyclic 5- to 15-membered ring selected from aryl, carbocyclyl, heteroaryl, or heterocyclyl;
X is a bond, —CH₂—, —C(CH₃)₂—, or —C(═O)—;
Y is a bond, —CH₂—, —NH—, or —O—;
W is a bond, —CH₂—, —CH₂CH₂—, —C(CH₃)H—, —N(R⁷)CO—, or —CONR⁷—;
Z is a bond —NH—, or —O—;
V is a bond, —CH₂(CR^{8a′}R^{9a′})—, or —(CR^{8a}R^{9a})ₘ—;
L is hydrogen, halogen, —CF₂R¹⁰, —CF₃, —CN, —OR¹⁰, —NR¹¹R¹², or —CONR¹¹R¹²;
R¹ and R² are each independently halogen, —CN, or —CF₃;
at least one R³ is —CH₂NHSO₂CH₃, —CH₂N(CH₃)SO₂CH₃, —NHSO₂CH₃, —NHSO₂CH₂CH₃, —SO₂NH₂, —SO₂CH₃, —NH₂, or —NH(C₁-C₃ alkyl); and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), or —N(CH₃)COO(C₁-C₃ alkyl);
R⁷ is hydrogen or C₁-C₃ alkyl;
R^{8a} and R^{9a} are each independently hydrogen, halogen, or C₁-C₃ alkyl;
R^{8a′} and R^{9a′} taken together form an optionally substituted 3- to 6-membered carbocyclyl or heterocyclyl containing one, two, or three heteroatoms selected from N, S, or O;
R¹⁰ is hydrogen, or C₁-C₃ alkyl;
R¹¹ and R¹² are each independently hydrogen or C₁-C₃ alkyl; or R¹¹ and R¹² taken together form an optionally substituted heterocyclyl which optionally contains one or two additional heteroatoms selected from N, S, or O;
m is 0, 1, or 2;
n1 and n2 are each independently 0, 1, or 2; and
n3 is 1, 2, 3, 4 or 5;
wherein C is not

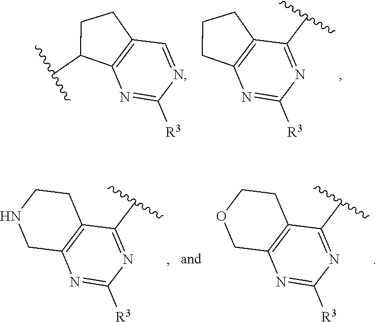

In one embodiment of the compounds of formula (G), C is a bridged bicyclic carbocycle, 6,6-fused heteroaryl or heterocycle, 5,6-fused heteroaryl or heterocycle, 6,5-fused heteroaryl or heterocycle, or 5,5-fused heteroaryl or heterocycle. In one embodiment, C is a 6,6-fused heteroaryl or heterocycle, 5,6-fused heteroaryl or heterocycle, 6,5-fused heteroaryl or heterocycle, or 5,5-fused heteroaryl or heterocycle.

In one embodiment of the compounds of formula (G), C is

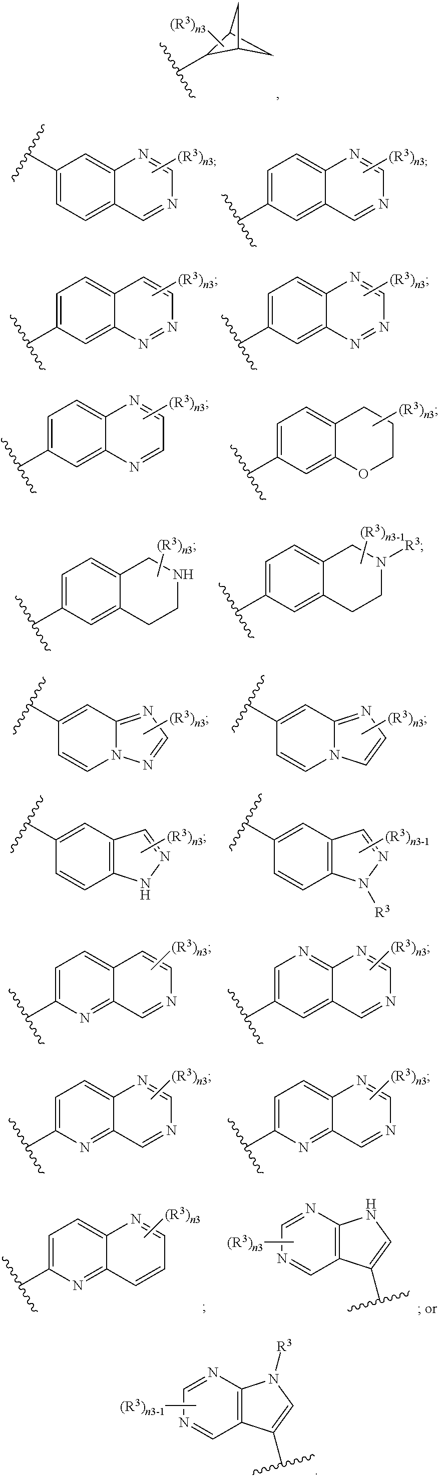

As used herein

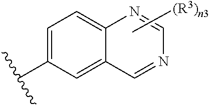

depicts where R³ substituent(s) can be on the phenyl portion of the fused ring, on the pyrimidine portion of the fused ring, and/or on both the phenyl portion and the pyrimidine portion of the fused ring. This type of depiction of substituents on a multi-cyclic ring applies throughout this disclosure.

In one embodiment of the compounds of formula (G), C is

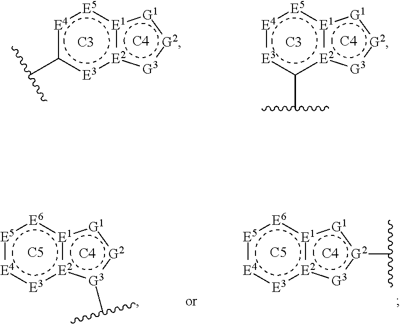

wherein:
ring C₃ and C₅ is aromatic;
E¹, E², E³, E⁴, E⁵, and E⁶ are each independently, C, CH, CR³ or N;
G¹, G², and G³, are each independently, CH, CH₂, CR³, C(R³)₂, O, S, N, NH, or NR³;
wherein at least two of E¹, E², E³, E⁴, and E⁵ in ring C₃ is C, CH, or CR³ (i.e., maximum 3 of E¹, E², E³, E⁴, and E⁵ is N);
wherein at least three of E¹, E², E³, E⁴, E⁵, and E⁶ in ring C₅ is C, CH, or CR³ (i.e., maximum 3 of E¹, E², E³, E⁴, E⁵, and E⁶ is N);
wherein at least two of E¹, E², G¹, G², and G³, is CH, CH₂, CR³, C(R³)₂ (i.e., maximum 3 of E¹, E², G¹, G², and G³ can be heteroatoms).

In one embodiment of the compounds of formula (G), C is

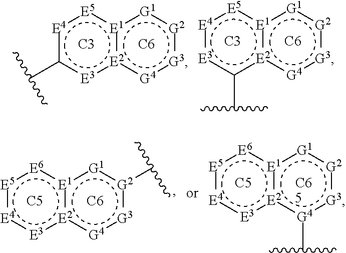

wherein:
ring C₃ and C₅ is aromatic;
E¹, E², E³, E⁴, E⁵ and E⁶ are each independently, C, CH, CR³ or N;
G¹, G², G³, and G⁴, are each independently, CH, CH₂, CR³, C(R³)₂, O, S, N, NH, or NR³;
wherein at least two of E¹, E², E³, E⁴, and E⁵ in ring C₃ is C, CH, or CR³;
wherein at least three of E¹, E², E³, E⁴, E⁵ and E⁶ in ring C₇ is C, CH, or CR³; and
wherein at least three of E¹, E², G¹, G², G³, and G⁴, is CH, CH₂, CR³, or C(R³)₂.

In one embodiment, ring C₃ or C₅ is phenyl. In one embodiment, ring C₃ or C₅ is a heteroaryl ring. In one embodiment, ring C₃ or C₅ is a pyridine, pyridazine, pyrimidine, pyrazine, 1,2,4-trizaine, or 1,3,5-triazine ring.

In one embodiment, ring C₄ is aromatic. In one embodiment, ring C₄ is a heteroaryl ring.

In one embodiment, ring C₄ is partially aromatic. In one embodiment, ring C₄ is a heterocyclyl or a carbocyclyl ring. In one embodiment, the bond between E¹-G¹, G¹-G², G²-G³, and G³-E² are each a single bond.

In one embodiment, E¹, E², E³, E⁴, E⁵ and E⁶ are each independently N, C, CH or CR². In one embodiment, E¹ and E² are C, and E³, E⁴, and E⁵ are each independently N, CH or CR³.

In one embodiment, ring C6 is partially aromatic. In one embodiment, ring C6 is a heterocyclyl or a carbocyclyl ring. In one embodiment, the bond between E¹-G¹, G¹-G², G²-G³, G³-G⁴ and G³-E² are each a single bond. In one embodiment, one of the bonds between E¹-G¹, G¹-G², G²-G³, G³-G⁴ and G³-E² is a double bond.

In one embodiment, E¹ and E² are each independently N or C and G¹, G², G³, and G⁴, are each independently N, NH, NR³, C, CH, CR³, O, or S.

In one embodiment, the present disclosure provides compounds of formula (H):

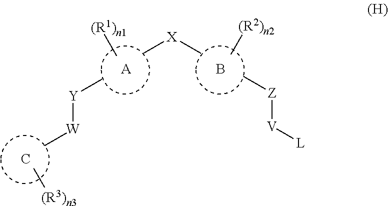

or a pharmaceutically acceptable salt, tautomer, stereoisomer or prodrug thereof, wherein:
A and B are each independently a phenyl or a pyridyl ring;
C is 5- or 6-membered heteroaryl, 4- to 6-membered heterocyclyl, phenyl, or bicyclic 5- or 6-membered carbocycle;
X is a bond, —CH₂—, —C(CH₃)₂—, or —C(═O)—;
Y is a bond, —CH₂—, —NH—, or —O—;
W is —CH₂—, —CH₂CH₂—, —C(CH₃)H—, —N(R⁷)CO—, or —CONR⁷—;
Z is a bond, —NH—, or —O—;
V is a bond, —CH₂(CR^{8a′}R^{9a′})—, or —(CR^{8a}R^{9a})ₘ—;
L is hydrogen, halogen, —CF₂R¹⁰, —CF₃, —CN, —OR¹⁰, —NR¹¹R¹², or —CONR¹¹R¹²;
R¹ and R² are each independently halogen, —CN, or —CF₃;
at least one R³ is —CH₂NHSO₂CH₃, —CH₂N(CH₃)SO₂CH₃, —NHSO₂CH₃, —NHSO₂CH₂CH₃, —SO₂NH₂, —SO₂CH₃, —NH₂, —NH(C₁-C₃ alkyl), —NHCOCF₃ or optionally substituted 5- or 6-membered heterocyclyl or heteroaryl; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), —N(CH₃)COO(C₁-C₃ alkyl); or optionally substituted 5- or 6-membered heterocyclyl;
R⁷ is hydrogen or C₁-C₃ alkyl;
R^{8a} and R^{9a} are each independently hydrogen, halogen, or C₁-C₃ alkyl;
R^{8a′} and R^{9a′} taken together form an optionally substituted 3- to 6-membered carbocyclyl or heterocyclyl containing one, two, or three heteroatoms selected from N, S, or O;
R¹⁰ is hydrogen, or C₁-C₃ alkyl;
R¹¹ and R¹² are each independently hydrogen or C₁-C₃ alkyl; or R¹¹ and R¹² taken together form an optionally substituted heterocyclyl which optionally contains one or two additional heteroatoms selected from N, S, or O;
m is 0, 1, or 2;
n1 and n2 are each independently 0, 1, or 2; and
n3 is 1, 2, 3, 4 or 5;
wherein:
a) at least one R³ is optionally substituted 4- or 6-membered heterocyclyl or heteroaryl, provided that 5- or 6-membered heterocyclyl is not

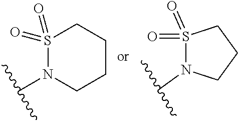 [SMILES: *N1CCCCS1(=O)=O ; *N1CCCS1(=O)=O]

when R³ is pyrimidyl; or
b) Z is a bond; V is a bond; L is —NR¹¹R¹²; and R¹¹ and R¹², taken together with the nitrogen atom to which it is attached, form an optionally substituted heterocyclic ring.

In one embodiment of the compounds of formula (H), C is a pyrimidine, pyrazine, pyridine, pyrazole, azetidine, phenyl, or bicyclo[1.1.1]pentane.

In one embodiment of the compounds of formula (H), R¹¹ and R¹², taken together with the nitrogen atom to which it is attached, form an optionally substituted piperidine or 2-azaspiro[3.3]heptane.

In one embodiment of the compounds of formula (H), at least one R³ is optionally substituted 4- or 6-membered heterocyclyl or heteroaryl containing one or two heteroatoms selected from N and O. In one embodiment, at least one R³ is an optionally substituted group selected from: pyrazolyl, morpholinyl, piperazinyl, piperidinyl, pyrrolidinyl, or azetidinyl. In one embodiment, at least one R³ is substituted with —OH, oxo, —CH₂NHSO₂CH₃, —CH₂N(CH₃)SO₂CH₃, —NHSO₂CH₃, —NHSO₂CH₂CH₃, —SO₂NH₂, —SO₂CH₃, —NH₂, —NH(C₁- C₃ alkyl), or —NHCOCF₃.

In one embodiment of the compounds of formula (H), Z is —NH— or —O—; V is —(CR^{8a}R^{9a})ₘ—; and L is halogen or H.

In one embodiment, the present disclosure provides compounds of formula (J):

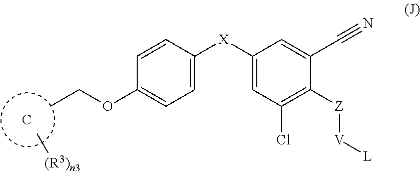

or a pharmaceutically acceptable salt, tautomer, stereoisomer or prodrug thereof, wherein:
C is pyrimidyl;
X is a bond, —CH₂— or —C(CH₃)₂—;
Z is —O— and —V-L is —CH₂CH(OH)CH₃, —CH₂CH(OH)CH₂Cl, or —CH₂CH₂CH₂Cl; or
Z is —NH—, V is —(CR^{8a}R^{9a})ₘ—, and L is hydrogen or halogen;
at least one R³ is —CH₂NHSO₂CH₃, —CH₂N(CH₃)SO₂CH₃, —NHSO₂CH₃, —NHSO₂CH₂CH₃, —SO₂NH₂, —SO₂CH₃, —NH₂, —NH(C₁-C₃ alkyl), —NHCOCF₃ or optionally substituted 5- or 6-membered heterocyclyl or heteroaryl; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), —N(CH₃)COO(C₁-C₃ alkyl); or optionally substituted 5- or 6-membered heterocyclyl;

R^{8a} and R^{9a} are each independently hydrogen, halogen, or C₁-C₃ alkyl;

m is 1, or 2; and

n3 is 1, 2, 3, 4 or 5.

In one embodiment, the present disclosure provides compounds of formula (K):

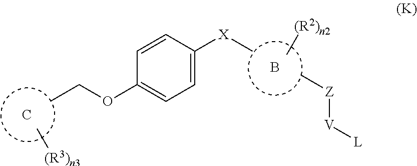

or a pharmaceutically acceptable salt, tautomer, stereoisomer or prodrug thereof, wherein:
B is pyridyl;
C is pyrimidyl;
X is a bond, —CH₂—, —C(CH₃)₂—, or —C(═O)—;
Z is a bond, —NH—, or —O—;
V is a bond, —CH₂(CR^{8a′}R^{9a′})—, or —(CR^{8a}R^{9a})ₘ—;
L is hydrogen, halogen, —CF₂R¹⁰, —CF₃, —CN, —OR¹⁰, —NR¹¹R¹², or —CONR¹¹R¹²;
R² is each independently halogen, —CN, or —CF₃;
at least one R³ is —CH₂NHSO₂CH₃, —CH₂N(CH₃)SO₂CH₃, —NHS₂CH₃, —NHSO₂CH₂CH₃, —S₂NH₂, —SO₂CH₃, —NH₂, —NH(C₁-C₃ alkyl), —NHCOCF₃ or optionally substituted 5- or 6-membered heterocyclyl or heteroaryl; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), —N(CH₃)COO(C₁-C₃ alkyl); or optionally substituted 5- or 6-membered heterocyclyl;
R^{8a} and R^{9a} are each independently hydrogen, halogen, or C₁-C₃ alkyl;
R^{8a′} and R^{9a′} taken together form an optionally substituted 3- to 6-membered carbocyclyl or heterocyclyl containing one, two, or three heteroatoms selected from N, S, or O;
R¹⁰ is hydrogen, or C₁-C₃ alkyl;
R¹¹ and R¹² are each independently hydrogen or C₁-C₃ alkyl; or R¹¹ and R¹² taken together form an optionally substituted heterocyclyl which optionally contains one or two additional heteroatoms selected from N, S, or O;
m is 1, 2, or 3;
n2 is 0, 1, or 2; and
n3 is 1, 2, 3, 4 or 5.

In one embodiment of the compounds of formula (K), n2 is 1 and R² is halogen.

In one embodiment of the compounds of formula (C), (E), (E-I), (F), (G), (H), (J), and/or (K), X is a bond, —CH₂— or —C(CH₃)₂—. In one embodiment, X is —C(CH₃)₂—.

In one embodiment of the compounds of formula (C), (E), (E-I), (F), (G), (H), (J), and/or (K), Y and Z are each —O—.

In one embodiment of the compounds of formula (E), (E-I), (F), (G), (H), and/or (K), —V-L is —CH₂CH₂Cl.

In one embodiment of the compounds of formula (C), (E), (E-I), (F), (G), and/or (H), —Y—W— is —OCH₂— or —OCH(CH₃)—.

In one embodiment of the compounds of formula (C), (E), (E-I), (F), (G), (H), and/or (K), L is a halogen.

In one embodiment of the compounds of formula (G) and (H):
Z is a bond —NH—, or —O—; and
V is —CH₂— and L is halogen, —CHCl₂, —CCl₃, or —CF₃; or
V is —CH₂CH₂— and L is halogen.

In one embodiment of the compounds of formula (C), (E), (E-I), (F), and/or (H), C is a pyrimidine, triazine, or thiophene ring.

In one embodiment of the compounds of formula (C), (E), (E-I), (F), and/or (H), C is

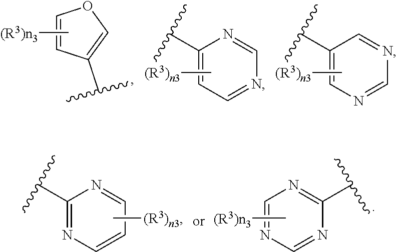

In one embodiment, C is

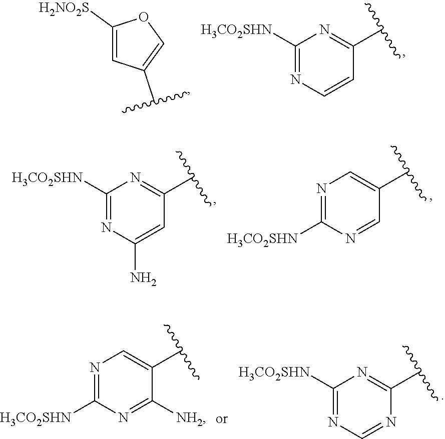

In one embodiment of the compounds of formula (C), (E), (E-I), (F), (H), (J), and/or (K), C is a pyrimidine ring. In one embodiment, C is

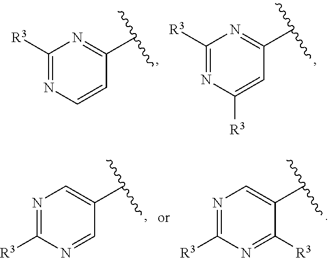

In one embodiment, C is

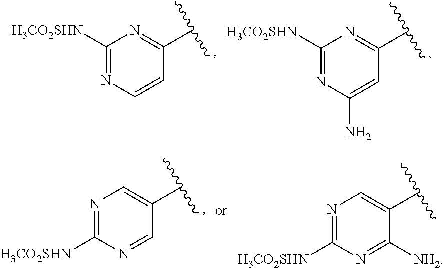

In one embodiment of the compounds of formula (E-I), (F), (G), (H), and/or (K), R¹ and R² are each independently Cl, —CN, or —CF₃. In one embodiment, R¹ and R² are each independently C₁ or —CN.

In one embodiment of the compounds of formula (C), (E), (E-I), (F), (G), (H), (J), and/or (K), R³ is each independently, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), —N(CH₃)COO(C₁-C₃ alkyl); or optionally substituted 5- or 6-membered heterocyclyl. In one embodiment, at least one R³ is —CH₂NHSO₂CH₃, —CH₂N(CH₃)SO₂CH₃, —NHSO₂CH₃, —NHSO₂CH₂CH₃, —SO₂NH₂, —SO₂CH₃, —NH₂, —NH(C₁- C₃ alkyl), or —NHCOCF₃; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), —N(CH₃)COO(C₁-C₃ alkyl); or optionally substituted 5- or 6-membered heterocyclyl.

In one embodiment of the compounds of formula (C), (E), (E-I), (F), (G), (H), (J), and/or (K), at least one R³ is —NHSO₂CH₃, —NHSO₂CH₂CH₃, —SO₂NH₂, or —SO₂CH₃. In one embodiment, at least one R³ is —NHSO₂CH₃, —NHSO₂CH₂CH₃, —SO₂NH₂, or —SO₂CH₃ and the other R³ is, if present, —CN, C₁-C₃ alkyl, C₁-C₃ alkoxy, —SO₂(C₁-C₃ alkyl), —NH₂, —(C₁-C₃ alkyl)NH₂, —NHSO₂CH₃, —N(CH₃)SO₂CH₃, —NHSO₂CH₂CH₃, —N(CH₃)SO₂CH₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl).

In one embodiment of the compounds of formula (C), (E), (E-I), (F), (G), (H), (J), and/or (K), R³ is —NHSO₂CH₃, —NHSO₂CH₂CH₃, —SO₂NH₂, —SO₂CH₃, or —NH₂.

In one embodiment, the present disclosure provides compounds of formula (D):

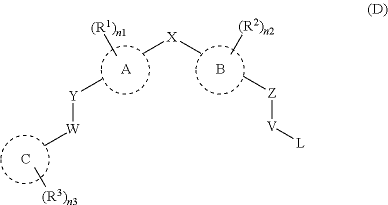

or a pharmaceutically acceptable salt, tautomer, stereoisomer or prodrug thereof, wherein:
A is absent;
B is 6- to 15-membered aryl ring;
C is a pyrimidine, triazine, or thiophene ring;
X is a bond, —CH₂—, —C(CH₃)₂—, or —C(═O)—;
Y is a bond or —O—;
W is —CH₂— or —C(CH₃)H—;
Z is a bond or —O—;
V is —CH₂— and L is halogen, —CHCl₂, —CCl₃, or —CF₃; or
V is —CH₂CH₂— and L is halogen;
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl);
n1 and n2 are each independently 0, 1, or 2; and
n3 is 1, 2, 3, 4 or 5.

In one embodiment of the compounds of formula (D), B is phenyl or fluorene ring, wherein each ring is optionally substituted with one or two R².

In one embodiment of the compounds of formula (D), X is a bond, —CH₂— or —C(CH₃)₂. In one embodiment, X is —C(CH₃)₂—.

In one embodiment of the compounds of formula (D), Y and Z are each —O—.

In one embodiment of the compounds of formula (D), —V-L is —CH₂CH₂Cl.

In one embodiment of the compounds of formula (D), —Y—W— is —OCH₂— or —OCH(CH₃)—.

In one embodiment of the compounds of formula (D), C is

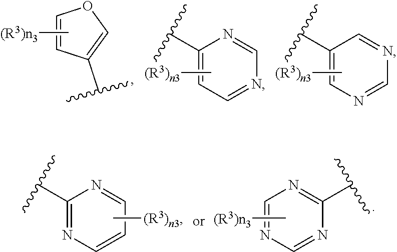

In one embodiment, C is

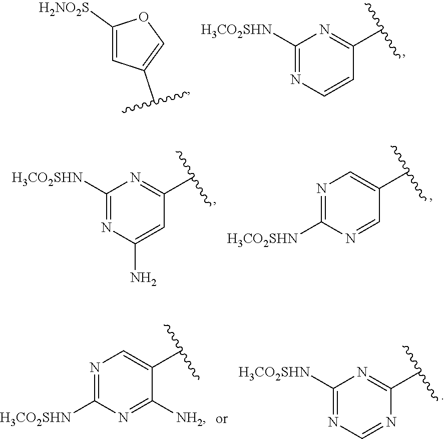

In one embodiment of the compounds of formula (D), C is a pyrimidine ring. In one embodiment, C is

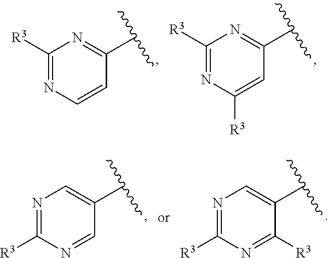

In one embodiment, C is

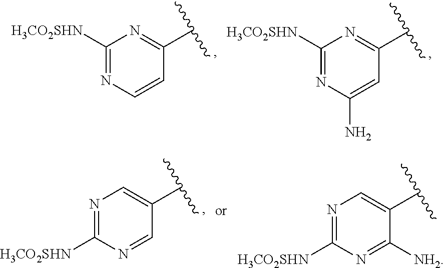

In one embodiment of the compounds of formula (D), R¹ and R² are each independently Cl, —CN, or —CF₃. In one embodiment, R¹ and R² are each independently C₁ or —CN.

In one embodiment of the compounds of formula (D), R³ is —NHSO₂CH₃, —NHSO₂CH₂CH₃, —SO₂NH₂, —SO₂CH₃, or —NH₂.

In one embodiment of the compounds of formula (D), at least one R³ is —NHSO₂CH₃, —NHSO₂CH₂CH₃, —SO₂NH₂, or —SO₂CH₃. In one embodiment, at least one R³ is —NHSO₂CH₃, —NHSO₂CH₂CH₃, —SO₂NH₂, or —SO₂CH₃ and the other R³ is, if present, —CN, C₁-C₃ alkyl, C₁-C₃ alkoxy, —SO₂(C₁-C₃ alkyl), —NH₂, —(C₁-C₃ alkyl)NH₂, —NHSO₂CH₃, —N(CH₃)SO₂CH₃, —NHSO₂CH₂CH₃, —N(CH₃)SO₂CH₂CH₃, —SO₂NH₂, —CONH₂, —CON(C₁-C₃ alkyl)₂, —CONH(C₁-C₃ alkyl), —NHCO(C₁-C₃ alkyl), —N(CH₃)COO(C₁-C₃ alkyl), —NHCO(C₁-C₃ alkyl), or —N(CH₃)COO(C₁-C₃ alkyl).

In one embodiment of the compounds of formula (I), (I-A), (I-B), (II), (A), (A-I), (A-II), (A-III), (B), (B-I), (C), (D), (E), (E-I), (F), (G), (H), (J), and/or (K), X is a bond, —CH₂—, —C(CH₃)H—, —C(CH₃)₂—, or —CH₂CH₂—. In one embodiment, X is —CH₂—, —C(CH₃)H—, or —C(CH₃)₂—. In some embodiments, X is —C(CH₃)₂—.

In one embodiment of the compounds of formula (I), (I-A), (I-B), (II), (A), (A-I), (A-II), (A-III), (B), (B-I), (C), (D), (E), (E-I), (F), (G), (H), and/or (K), n1 is 0, 1, or 2. In some embodiments, n1 is 0 or 1. In other embodiments, n1 is 0. In some embodiments, n1 is 1. In one embodiment, the sum of n1 and n2 is 0, 1, 2, 3, or 4. In some embodiments, the sum of n1 and n2 is 1, 2, 3, or 4. In one embodiment, the sum of n1 and n2 is 2.

In one embodiment of the compounds of formula (I), (I-A), (I-B), (II), (A), (A-I), (A-II), (A-III), (B), (B-I), (C), (D), (E), (E-I), (F), (G), (H), and/or (K), n2 is 0, 1, or 2. In some embodiments, n2 is 1 or 2. In other embodiments, n2 is 0. In some embodiments, n2 is 1. In some embodiments, n2 is 2.

In some embodiments of the compounds of formula (I), (I-A), (I-B), (II), (A), (A-I), (A-II), (A-III), (B), (B-I), (C), (D), (E), (E-I), (F), (G), (H), (J), and/or (K), n3 is 1, 2, 3, 4, or 5. In some embodiments, n3 is 1, 2, 3, or 4. In one embodiment, n3 is 1, 2, or 3. In one embodiment, n3 is 1 or 2.

In one embodiment of the compounds of formula (I), (I-A), (I-B), (II), (A), (A-I), (A-II), (A-III), (B), (B-I), (C), (D), (E), (E-I), (F), (G), (H), (J), and/or (K), optional substituent is selected from halogen, —CN, —CF₃, —OH, —S(C₁-C₃ alkyl), C₁-C₃ alkyl, C₂-C₃ alkenyl, C₂-C₃ alkynyl, C₁-C₃ alkoxy, —(C₁-C₃ alkyl)-(C₁-C₃ alkoxy), —(C₁-C₃ alkyl)-OH, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —(C₁-C₆ alkyl)- NR¹⁴COR¹⁶, —CONR¹⁴R¹⁵, —(C₁-C₃ alkyl)-CONR¹⁴R¹⁵, —SO₂NR¹⁴R¹⁵, —(C₁-C₃ alkyl)-SO₂NR¹⁴R¹⁵, —SO(C₁-C₃ alkyl), —SO₂(C₁-C₃ alkyl), or —(C₁-C₆ alkyl)-SO₂(C₁-C₃ alkyl). In another embodiment, the optional substituent is halogen, —CN, —CF₃, —OH, C₁-C₃ alkyl, C₁-C₃ alkoxy, —NH₂, —SCH₃, —SO₂CH₃, —NHSO₂CH₃, —CH₂NHSO₂CH₃, —SO₂NH₂, —CONH₂, or —NHCOCH₃. In another embodiment, the optional substituent is halogen, —CN, —CF₃, —OH, C₁-C₃ alkyl, C₁-C₃ alkyl-OH, C₁-C₃ alkoxy, —NH₂, —SCH₃, —SO₂CH₃, —NHSO₂CH₃, —CH₂NHSO₂CH₃, —SO₂NH₂, —CONH₂, or —NHCOCH₃.

In some embodiments of the compounds of formula (I), (I-A), (I-B), (II), (A), (A-I), (A-II), (A-III), (B), (B-I), (C), (D), (E), (E-I), (F), (G), (H), (J), and/or (K), a hydrogen atom can be replaced with a deuterium atom.

In one embodiment of the compound of formula (I), (I-A), (I-B), (II), (A), (A-I), (A-II), (A-III), (B), (B-I), (C), (D), (E), (E-I), (F), (G), (H), (J), and/or (K), the compound excludes Compounds A1-A234 disclosed in WO 2020/081999. In one embodiment of the compound of formula (I), (I-A), (I-B), (II), (A), (A-I), (A-II), (A-III), (B), (B-I), (C), (D), (E), (E-I), (F), (G), (H), (J), and/or (K), the compound excludes Compounds presented in Table A of WO 2020/081999.

In one embodiment of the compound of formula (I), (I-A), (I-B), (II), (A), (A-I), (A-II), (A-III), (B), (B-I), (C), (D), (E), (E-I), (F), (G), (H), (J), and/or (K), the compound is selected from Table A below, or a pharmaceutically acceptable salt, tautomer, stereoisomer or prodrug thereof. In one embodiment of the present disclosure, the compound is selected from Compounds A1-A199, or a pharmaceutically acceptable salt, tautomer, stereoisomer or prodrug thereof.

In one embodiment of the compound of formula (A), the compound is Compound A7, A8, A9, A10, A17, A18, A19, A20, A21, A22 A23, A24, A28, A29, A30, A31, A32, A33, A34, A35, A35(R), A35(S), A36, or A40, or a pharmaceutically acceptable salt, tautomer, stereoisomer or prodrug thereof. In one embodiment of the compound of formula (A), (A-I), or (A-II) the compound is Compound A7, A8, A9, A10, A17, A18, A19, A20, A21, A22 A23, A24, A28, A29, A30, A31, A32, A33, A34, A35, A35(R), A35(S), A36, A40, A44, A45, A46, A52, A53, A55, A56, A57, A58, A59, A60, A61, A62, A63, A64, A65, A69, A70, A71, A73, A74, A77, A81, A82, A83, A84, A85, A115, A122, A123, A124, A125, A126, A131, A133, A134, A135, A136, A137, A138, A139, A140, A143, A144, A146, A147, A148, A153, A154, A155, A156, A157, A158, A159, A160, A161, A162, A163, A164, A165, A167, A168, A169, A170, A171, A180, A185, A186, A187, A189, A190, A200, A201, A202, A204, A205, A206, A207, A208, A209, A210, A218, A220, A221, A222, A223, A224, A225, A226, A227, A233, A234, A236, A238, A239, A241, A242, A244, A245, A246, A247, A248, A249, A266, A270, or A276, or a pharmaceutically acceptable salt, tautomer, stereoisomer or prodrug thereof.

In one embodiment of the compound of formula (A-III), the compound is A72, A78, A79, A80, A128, A145, or A184, or a pharmaceutically acceptable salt, tautomer, stereoisomer or prodrug thereof.

In one embodiment of the compound of formula (B), the compound is Compound A3, A11, A12, A13, A14, A15, A16, A25, A26, A27, or A38, or a pharmaceutically acceptable salt, tautomer, stereoisomer or prodrug thereof. In one embodiment of the compound of formula (B) or (B-I), the compound is Compound A3, A11, A12, A13, A14, A15, A16, A25, A26, A27, A38, or A75, or a pharmaceutically acceptable salt, tautomer, stereoisomer or prodrug thereof.

In one embodiment of the compound of formula (C), the compound is Compound A1 or A2, or a pharmaceutically acceptable salt, tautomer, stereoisomer or prodrug thereof.

In one embodiment of the compound of formula (D), the compound is Compound A4 or A5, or a pharmaceutically acceptable salt, tautomer, stereoisomer or prodrug thereof.

In one embodiment of the compound of formula (E), the compound is Compound A6, A37, or A41, or a pharmaceutically acceptable salt, tautomer, stereoisomer or prodrug thereof. In one embodiment of the compound of formula (E) and/or (E-I), the compound is Compound A6, A37, A41, A141, A142, A182, A183, or A188, or a pharmaceutically acceptable salt, tautomer, stereoisomer or prodrug thereof.

In one embodiment of the compound of formula (F), the compound is Compound A39 or a pharmaceutically acceptable salt, tautomer, stereoisomer or prodrug thereof.

In one embodiment of the compound of formula (G), the compound is Compound A51, A107, A108, A109, A111, A112, A113, A114, A117, A121, A132, A149, A152, A172, A173, A175, A176, A177, A191, A192, A193, A194, A195, A196, A197, A198, A199, A203, A211, A212, A213, A214, A215, A216, A217, A219, A228, A229, A230, A232, A243, A250, A254, A255, A256, A257, A263, A264, A265, A268, A269, A271, A272, A273, A274, A275, A277, A278, A279, A280, A281, A282, A284, or A285, or a pharmaceutically acceptable salt, tautomer, stereoisomer or prodrug thereof.

In one embodiment of the compound of formula (H), the compound is Compound A42, A90, A99, A100, A101, A105, A106, A118, A119, A120, A151, A166, A178, A179, A240, A251, A252, A253, A259, A260, A261, A262, A267, or A283, or a pharmaceutically acceptable salt, tautomer, stereoisomer or prodrug thereof.

In one embodiment of the compound of formula (J), the compound is Compound A66, A129, A130, A150, A174, A181, or A258, or a pharmaceutically acceptable salt, tautomer, stereoisomer or prodrug thereof.

In one embodiment of the compound of formula (K), the compound is Compound A67, A103, or A104, or a pharmaceutically acceptable salt, tautomer, stereoisomer or prodrug thereof.

In one embodiment, the compound of the disclosure is Compound A43, A47, A48, A49, A50, A68, A76, A86, A87, A88, A89, A91, A92, A93, A94, A95, A96, A97, A98, A102, A127, or A231, or a pharmaceutically acceptable salt, tautomer, stereoisomer or prodrug thereof.

TABLE A
Compounds

| Compound | |
|---|---|
| ID | Structure |
| A1 | 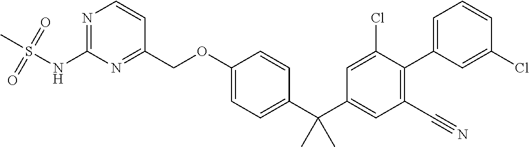 [SMILES: CC(C)(c1ccc(OCc2ccnc(NS(C)(=O)=O)n2)cc1)c1cc(Cl)c(-c2cccc(Cl)c2)c(C#N)c1] |
| A2 | 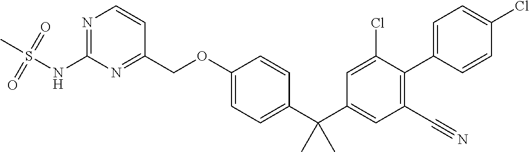 [SMILES: CC(C)(c1ccc(OCc2ccnc(NS(C)(=O)=O)n2)cc1)c1cc(Cl)c(-c2ccc(Cl)cc2)c(C#N)c1] |
| A3 | 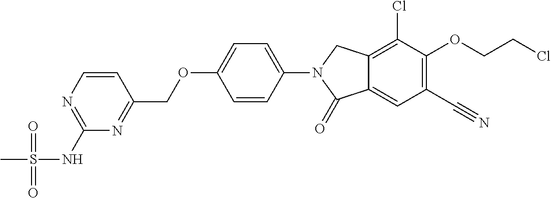 [SMILES: CS(=O)(=O)Nc1nccc(COc2ccc(N3Cc4c(cc(C#N)c(OCCCl)c4Cl)C3=O)cc2)n1] |
| A4 | 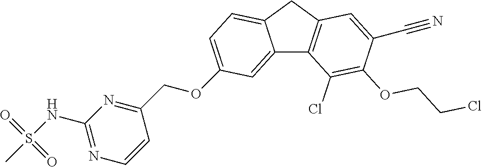 [SMILES: CS(=O)(=O)Nc1nccc(COc2ccc3c(c2)-c2c(cc(C#N)c(OCCCl)c2Cl)C3)n1] |
| A5 | 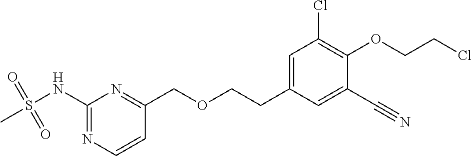 [SMILES: CS(=O)(=O)Nc1nccc(COCCc2cc(Cl)c(OCCCl)c(C#N)c2)n1] |
| A6 | 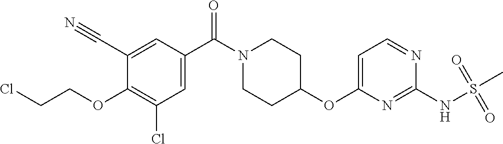 [SMILES: CS(=O)(=O)Nc1nccc(OC2CCN(C(=O)c3cc(Cl)c(OCCCl)c(C#N)c3)CC2)n1] |
| A7 | 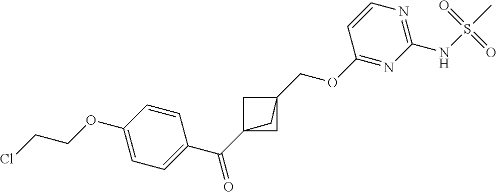 [SMILES: CS(=O)(=O)Nc1nccc(OCC23CC(C(=O)c4ccc(OCCCl)cc4)(C2)C3)n1] |
| A8 | 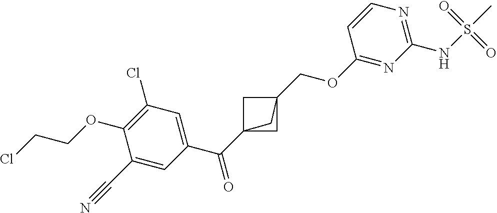 [SMILES: CS(=O)(=O)Nc1nccc(OCC23CC(C(=O)c4cc(Cl)c(OCCCl)c(C#N)c4)(C2)C3)n1] |
| A9 | 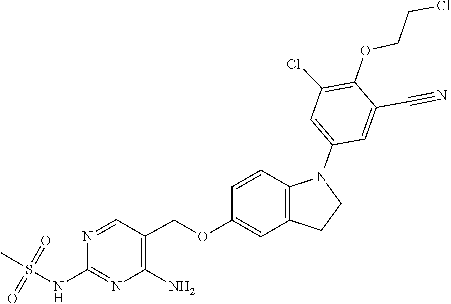 [SMILES: CS(=O)(=O)Nc1ncc(COc2ccc3c(c2)CCN3c2cc(Cl)c(OCCCl)c(C#N)c2)c(N)n1] |
| A10 | 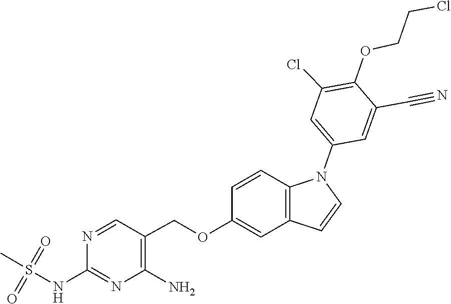 [SMILES: CS(=O)(=O)Nc1ncc(COc2ccc3c(ccn3-c3cc(Cl)c(OCCCl)c(C#N)c3)c2)c(N)n1] |
| A11 | 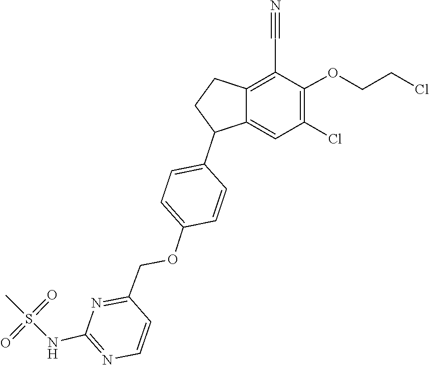 [SMILES: CS(=O)(=O)Nc1nccc(COc2ccc(C3CCc4c3cc(Cl)c(OCCCl)c4C#N)cc2)n1] |
| A12 | 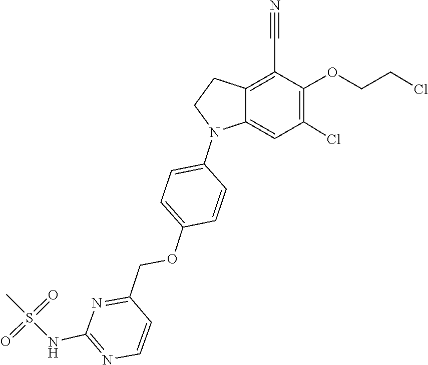 [SMILES: CS(=O)(=O)Nc1nccc(COc2ccc(N3CCc4c3cc(Cl)c(OCCCl)c4C#N)cc2)n1] |
| A13 | 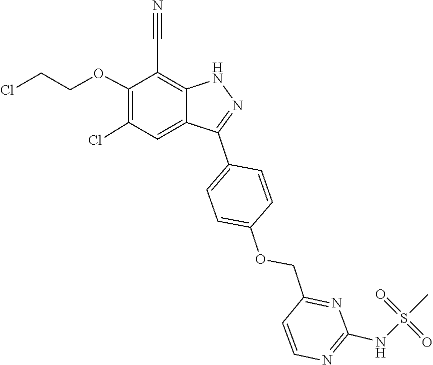 [SMILES: CS(=O)(=O)Nc1nccc(COc2ccc(-c3n[nH]c4c(C#N)c(OCCCl)c(Cl)cc34)cc2)n1] |
| A14 | 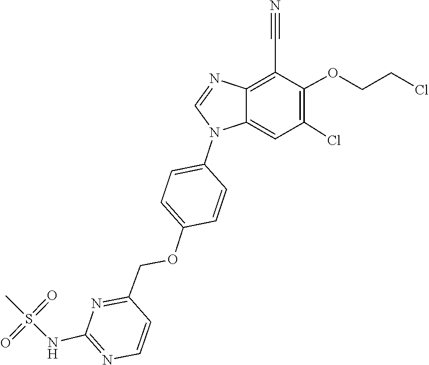 [SMILES: CS(=O)(=O)Nc1nccc(COc2ccc(-n3cnc4c(C#N)c(OCCCl)c(Cl)cc43)cc2)n1] |
| A15 | 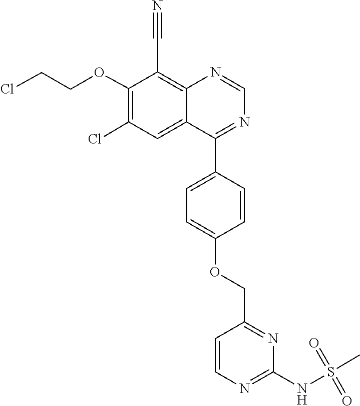 [SMILES: CS(=O)(=O)Nc1nccc(COc2ccc(-c3ncnc4c(C#N)c(OCCCl)c(Cl)cc34)cc2)n1] |
| A16 | 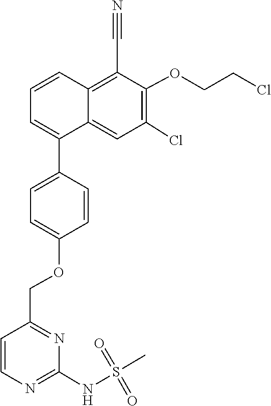 [SMILES: CS(=O)(=O)Nc1nccc(COc2ccc(-c3cccc4c(C#N)c(OCCCl)c(Cl)cc34)cc2)n1] |
| A17 | 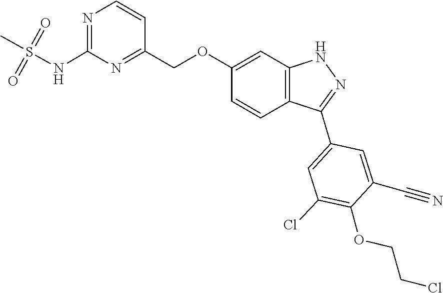 [SMILES: CS(=O)(=O)Nc1nccc(COc2ccc3c(-c4cc(Cl)c(OCCCl)c(C#N)c4)n[nH]c3c2)n1] |
| A18 | 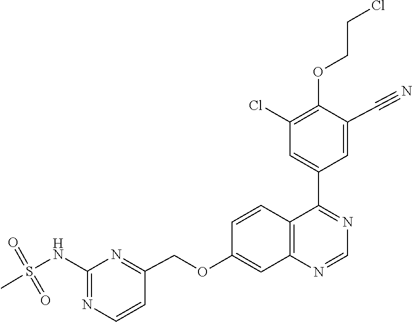 [SMILES: CS(=O)(=O)Nc1nccc(COc2ccc3c(-c4cc(Cl)c(OCCCl)c(C#N)c4)ncnc3c2)n1] |
| A19 | 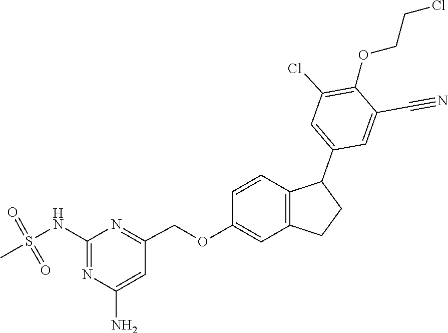 [SMILES: CS(=O)(=O)Nc1nc(N)cc(COc2ccc3c(c2)CCC3c2cc(Cl)c(OCCCl)c(C#N)c2)n1] |
| A20 | 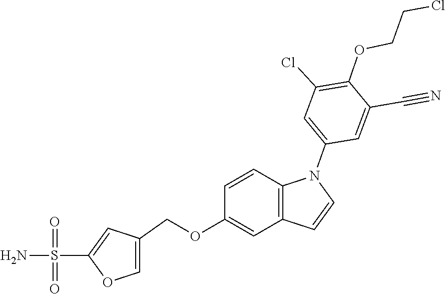 [SMILES: N#Cc1cc(-n2ccc3cc(OCc4coc(S(N)(=O)=O)c4)ccc32)cc(Cl)c1OCCCl] |
| A21 | 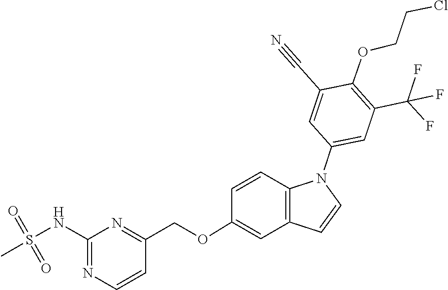 [SMILES: CS(=O)(=O)Nc1nccc(COc2ccc3c(ccn3-c3cc(C#N)c(OCCCl)c(C(F)(F)F)c3)c2)n1] |
| A22 | 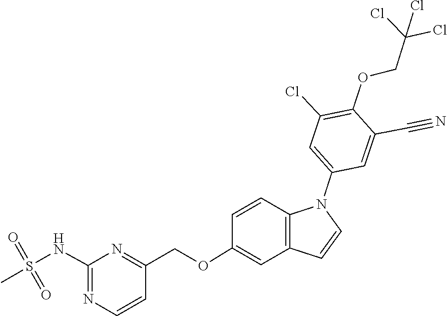 [SMILES: CS(=O)(=O)Nc1nccc(COc2ccc3c(ccn3-c3cc(Cl)c(OCC(Cl)(Cl)Cl)c(C#N)c3)c2)n1] |
| A23 | 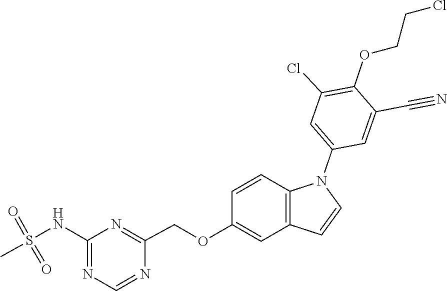 [SMILES: CS(=O)(=O)Nc1ncnc(COc2ccc3c(ccn3-c3cc(Cl)c(OCCCl)c(C#N)c3)c2)n1] |
| A24 | 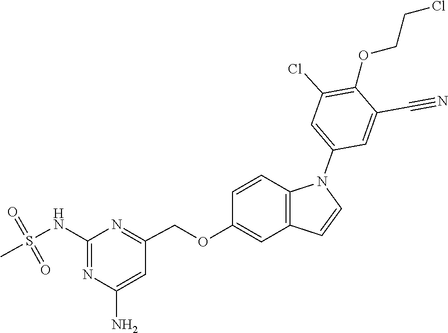 [SMILES: CS(=O)(=O)Nc1nc(N)cc(COc2ccc3c(ccn3-c3cc(Cl)c(OCCCl)c(C#N)c3)c2)n1] |
| A25 | 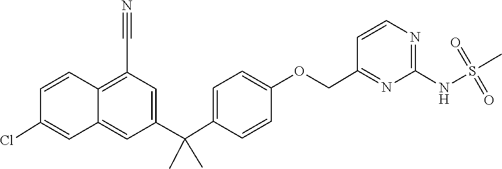 [SMILES: CC(C)(c1ccc(OCc2ccnc(NS(C)(=O)=O)n2)cc1)c1cc(C#N)c2ccc(Cl)cc2c1] |
| A26 | 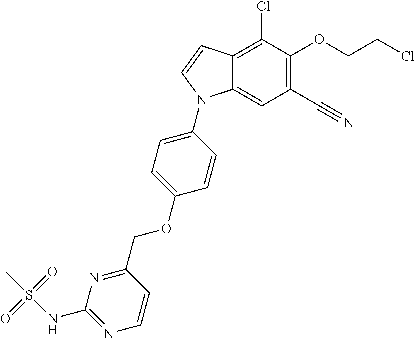 [SMILES: CS(=O)(=O)Nc1nccc(COc2ccc(-n3ccc4c(Cl)c(OCCCl)c(C#N)cc43)cc2)n1] |
| A27 | 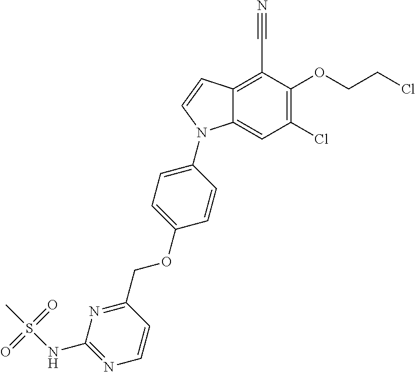 [SMILES: CS(=O)(=O)Nc1nccc(COc2ccc(-n3ccc4c(C#N)c(OCCCl)c(Cl)cc43)cc2)n1] |
| A28 | 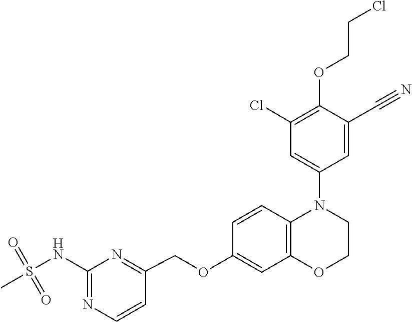 [SMILES: CS(=O)(=O)Nc1nccc(COc2ccc3c(c2)OCCN3c2cc(Cl)c(OCCCl)c(C#N)c2)n1] |
| A29 | 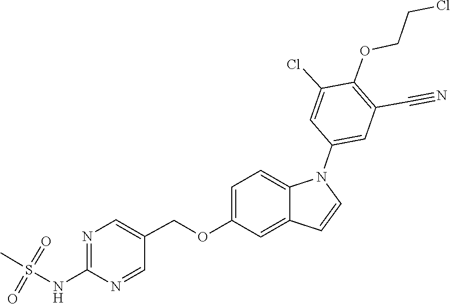 [SMILES: CS(=O)(=O)Nc1ncc(COc2ccc3c(ccn3-c3cc(Cl)c(OCCCl)c(C#N)c3)c2)cn1] |
| A30 | 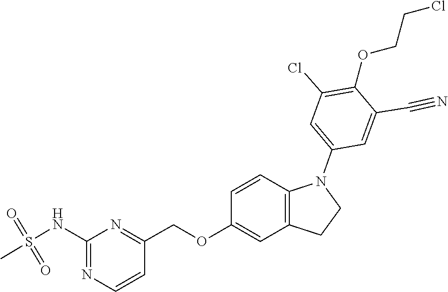 [SMILES: CS(=O)(=O)Nc1nccc(COc2ccc3c(c2)CCN3c2cc(Cl)c(OCCCl)c(C#N)c2)n1] |
| A31 | 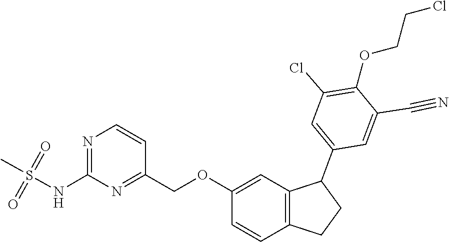 [SMILES: CS(=O)(=O)Nc1nccc(COc2ccc3c(c2)C(c2cc(Cl)c(OCCCl)c(C#N)c2)CC3)n1] |
| A32 | 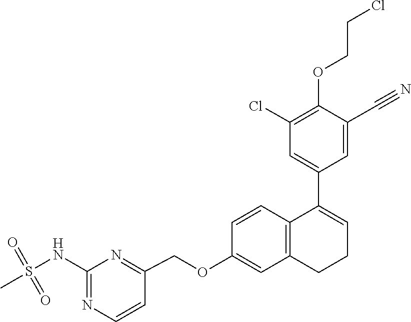 [SMILES: CS(=O)(=O)Nc1nccc(COc2ccc3c(c2)CCC=C3c2cc(Cl)c(OCCCl)c(C#N)c2)n1] |
| A33 | 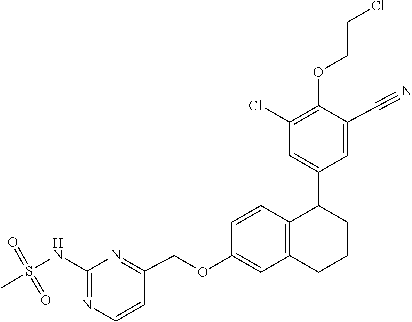 [SMILES: CS(=O)(=O)Nc1nccc(COc2ccc3c(c2)CCCC3c2cc(Cl)c(OCCCl)c(C#N)c2)n1] |
| A34 | 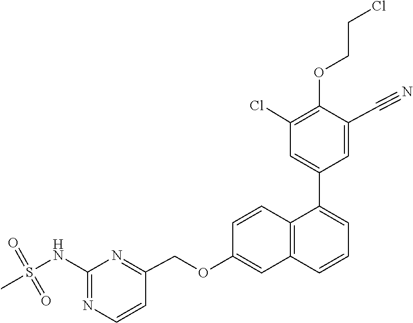 [SMILES: CS(=O)(=O)Nc1nccc(COc2ccc3c(-c4cc(Cl)c(OCCCl)c(C#N)c4)cccc3c2)n1] |
| A35 | 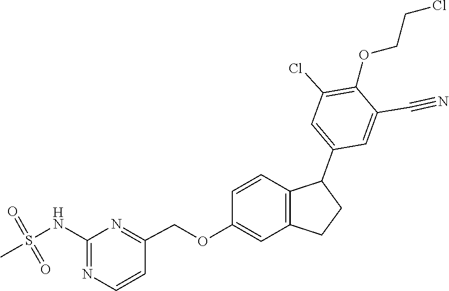 [SMILES: CS(=O)(=O)Nc1nccc(COc2ccc3c(c2)CCC3c2cc(Cl)c(OCCCl)c(C#N)c2)n1] |
| A35(S) | 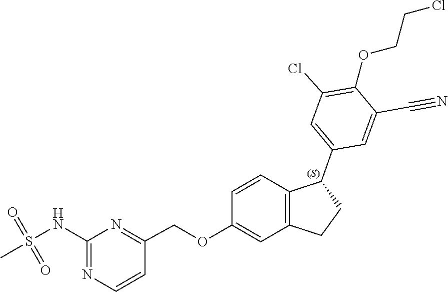 |
| A35(R) | 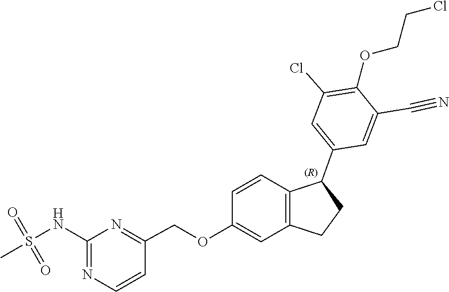 |
| A36 | 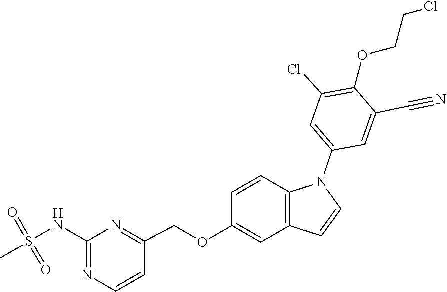 [SMILES: CS(=O)(=O)Nc1nccc(COc2ccc3c(ccn3-c3cc(Cl)c(OCCCl)c(C#N)c3)c2)n1] |
| A37 | 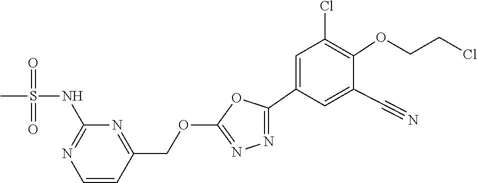 [SMILES: CS(=O)(=O)Nc1nccc(COc2nnc(-c3cc(Cl)c(OCCCl)c(C#N)c3)o2)n1] |
| A38 | 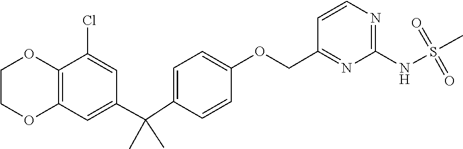 [SMILES: CC(C)(c1ccc(OCc2ccnc(NS(C)(=O)=O)n2)cc1)c1cc(Cl)c2c(c1)OCCO2] |
| A39 | 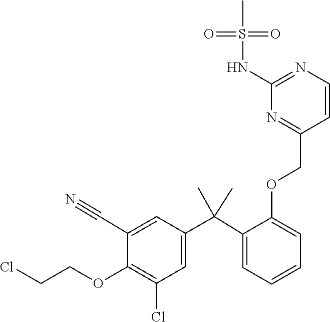 [SMILES: CC(C)(c1cc(Cl)c(OCCCl)c(C#N)c1)c1ccccc1OCc1ccnc(NS(C)(=O)=O)n1] |
| A40 | 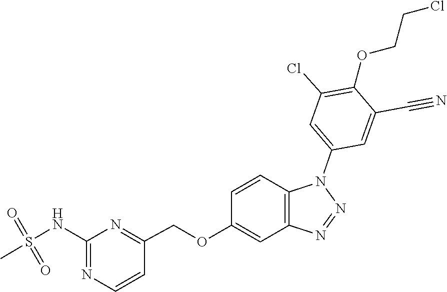 [SMILES: CS(=O)(=O)Nc1nccc(COc2ccc3c(c2)nnn3-c2cc(Cl)c(OCCCl)c(C#N)c2)n1] |
| A41 | 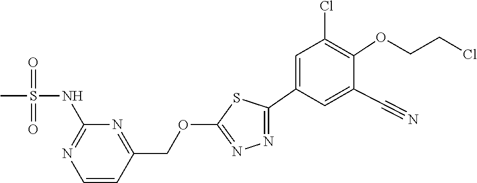 [SMILES: CS(=O)(=O)Nc1nccc(COc2nnc(-c3cc(Cl)c(OCCCl)c(C#N)c3)s2)n1] |
| A42 | 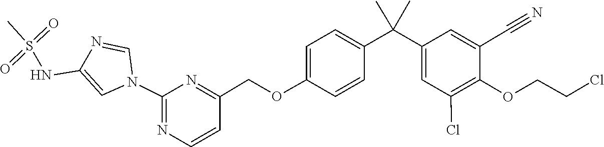 [SMILES: CC(C)(c1ccc(OCc2ccnc(-n3cnc(NS(C)(=O)=O)c3)n2)cc1)c1cc(Cl)c(OCCCl)c(C#N)c1] |
| A43 | 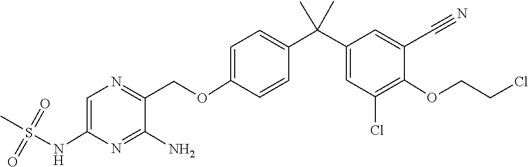 [SMILES: CC(C)(c1ccc(OCc2ncc(NS(C)(=O)=O)nc2N)cc1)c1cc(Cl)c(OCCCl)c(C#N)c1] |
| A44 | 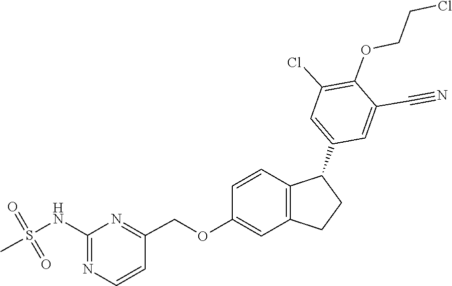 [SMILES: CS(=O)(=O)Nc1nccc(COc2ccc3c(c2)CC[C@H]3c2cc(Cl)c(OCCCl)c(C#N)c2)n1] |
| A45 | 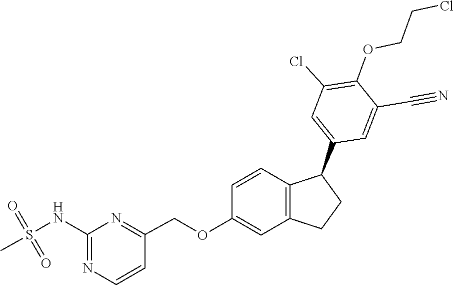 [SMILES: CS(=O)(=O)Nc1nccc(COc2ccc3c(c2)CC[C@@H]3c2cc(Cl)c(OCCCl)c(C#N)c2)n1] |
| A46 | 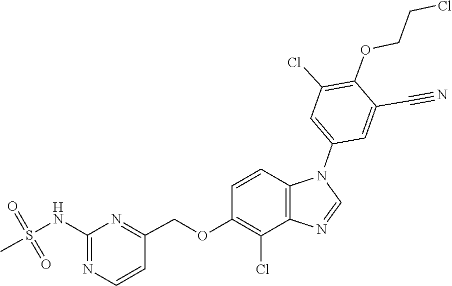 [SMILES: CS(=O)(=O)Nc1nccc(COc2ccc3c(ncn3-c3cc(Cl)c(OCCCl)c(C#N)c3)c2Cl)n1] |
| A47 | 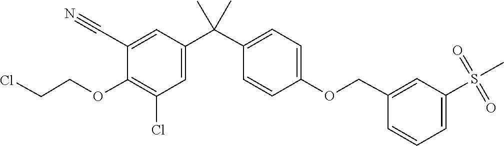 [SMILES: CC(C)(c1ccc(OCc2cccc(S(C)(=O)=O)c2)cc1)c1cc(Cl)c(OCCCl)c(C#N)c1] |
| A48 | 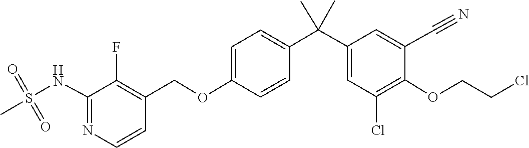 [SMILES: CC(C)(c1ccc(OCc2ccnc(NS(C)(=O)=O)c2F)cc1)c1cc(Cl)c(OCCCl)c(C#N)c1] |
| A49 | 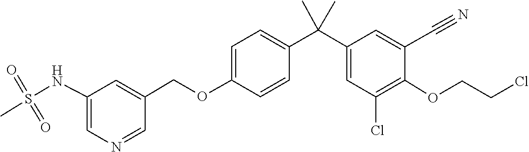 [SMILES: CC(C)(c1ccc(OCc2cncc(NS(C)(=O)=O)c2)cc1)c1cc(Cl)c(OCCCl)c(C#N)c1] |
| A50 | 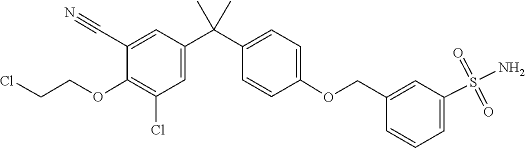 [SMILES: CC(C)(c1ccc(OCc2cccc(S(N)(=O)=O)c2)cc1)c1cc(Cl)c(OCCCl)c(C#N)c1] |
| A51 | 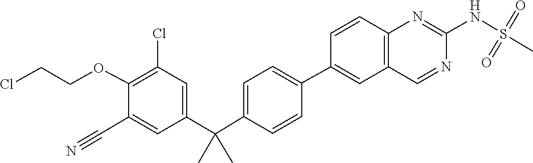 [SMILES: CC(C)(c1ccc(-c2ccc3nc(NS(C)(=O)=O)ncc3c2)cc1)c1cc(Cl)c(OCCCl)c(C#N)c1] |
| A52 | 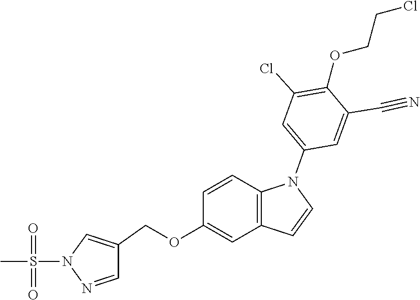 [SMILES: CS(=O)(=O)n1cc(COc2ccc3c(ccn3-c3cc(Cl)c(OCCCl)c(C#N)c3)c2)cn1] |
| A53 | 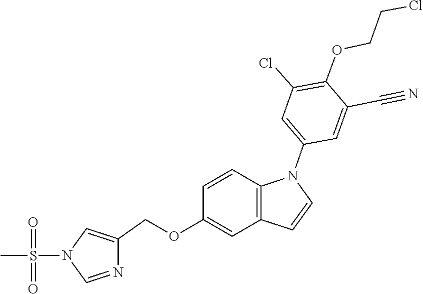 [SMILES: CS(=O)(=O)n1cnc(COc2ccc3c(ccn3-c3cc(Cl)c(OCCCl)c(C#N)c3)c2)c1] |
| A54 | 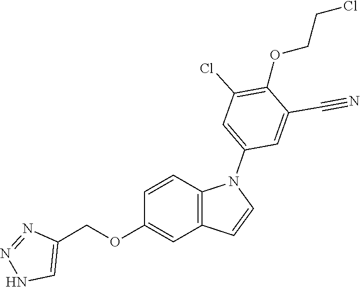 [SMILES: N#Cc1cc(-n2ccc3cc(OCc4c[nH]nn4)ccc32)cc(Cl)c1OCCCl] |
| A55 | 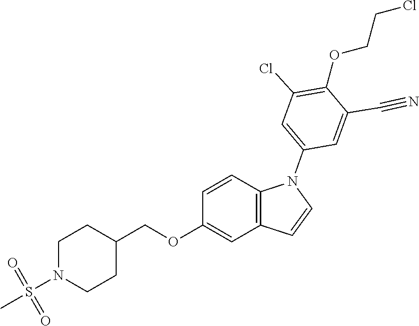 [SMILES: CS(=O)(=O)N1CCC(COc2ccc3c(ccn3-c3cc(Cl)c(OCCCl)c(C#N)c3)c2)CC1] |
| A56 | 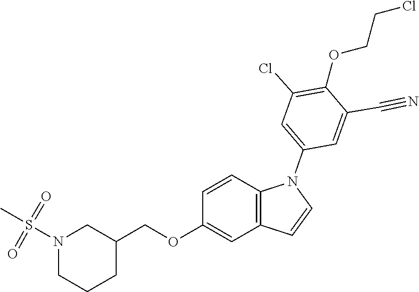 [SMILES: CS(=O)(=O)N1CCCC(COc2ccc3c(ccn3-c3cc(Cl)c(OCCCl)c(C#N)c3)c2)C1] |
| A57 | 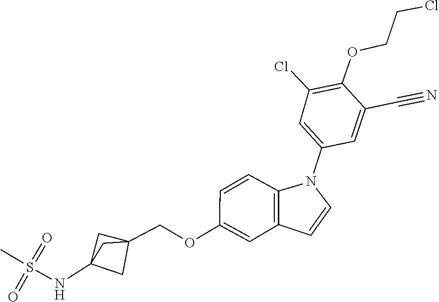 [SMILES: CS(=O)(=O)NC12CC(COc3ccc4c(ccn4-c4cc(Cl)c(OCCCl)c(C#N)c4)c3)(C1)C2] |
| A58 | 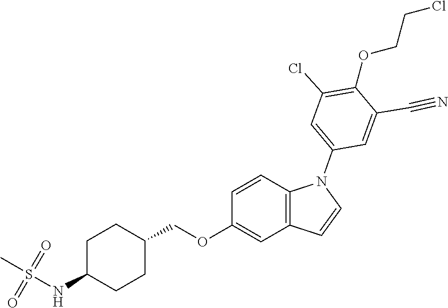 [SMILES: CS(=O)(=O)N[C@H]1CC[C@H](COc2ccc3c(ccn3-c3cc(Cl)c(OCCCl)c(C#N)c3)c2)CC1] |
| A59 | 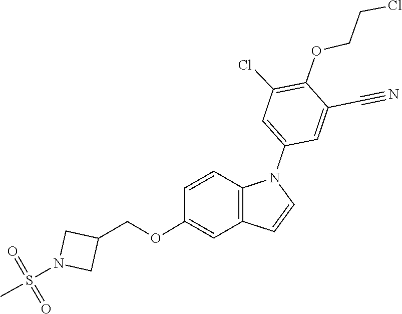 [SMILES: CS(=O)(=O)N1CC(COc2ccc3c(ccn3-c3cc(Cl)c(OCCCl)c(C#N)c3)c2)C1] |
| A60 | 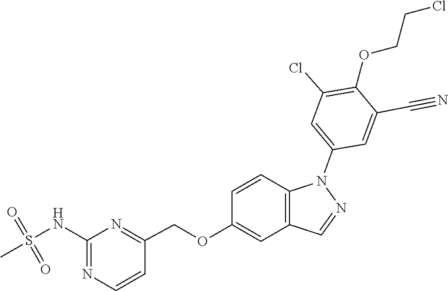 [SMILES: CS(=O)(=O)Nc1nccc(COc2ccc3c(cnn3-c3cc(Cl)c(OCCCl)c(C#N)c3)c2)n1] |
| A61 | 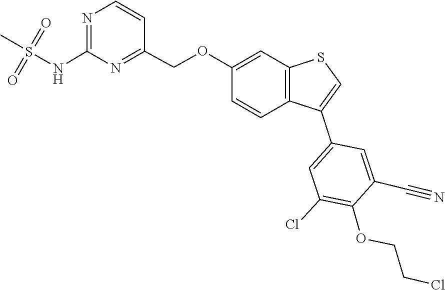 [SMILES: CS(=O)(=O)Nc1nccc(COc2ccc3c(-c4cc(Cl)c(OCCCl)c(C#N)c4)csc3c2)n1] |
| A62 | 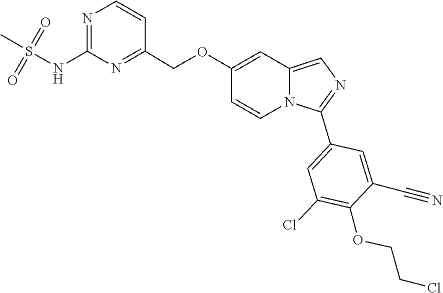 [SMILES: CS(=O)(=O)Nc1nccc(COc2ccn3c(-c4cc(Cl)c(OCCCl)c(C#N)c4)ncc3c2)n1] |
| A63 | 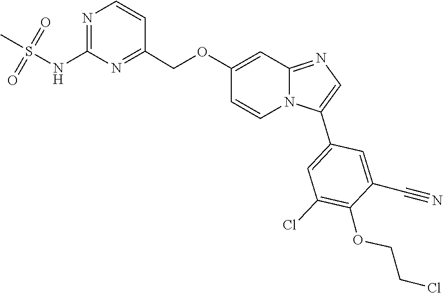 [SMILES: CS(=O)(=O)Nc1nccc(COc2ccn3c(-c4cc(Cl)c(OCCCl)c(C#N)c4)cnc3c2)n1] |
| A64 | 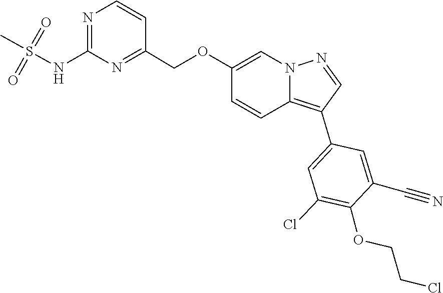 [SMILES: CS(=O)(=O)Nc1nccc(COc2ccc3c(-c4cc(Cl)c(OCCCl)c(C#N)c4)cnn3c2)n1] |
| A65 | 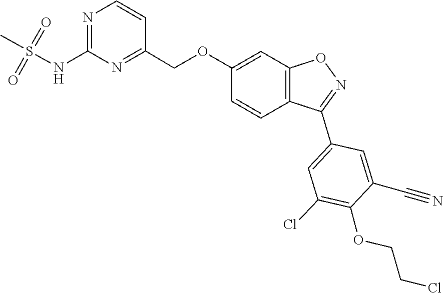 [SMILES: CS(=O)(=O)Nc1nccc(COc2ccc3c(-c4cc(Cl)c(OCCCl)c(C#N)c4)noc3c2)n1] |
| A66 | 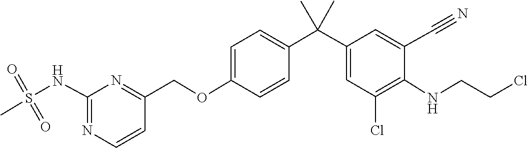 [SMILES: CC(C)(c1ccc(OCc2ccnc(NS(C)(=O)=O)n2)cc1)c1cc(Cl)c(NCCCl)c(C#N)c1] |
| A67 | 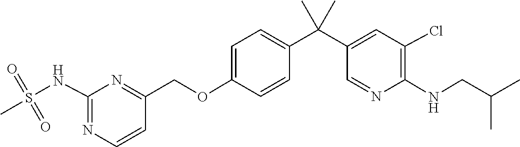 [SMILES: CC(C)CNc1ncc(C(C)(C)c2ccc(OCc3ccnc(NS(C)(=O)=O)n3)cc2)cc1Cl] |
| A68 | 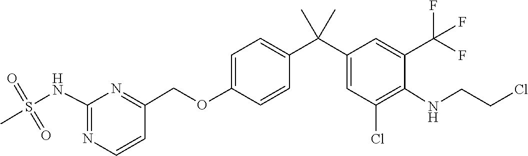 [SMILES: CC(C)(c1ccc(OCc2ccnc(NS(C)(=O)=O)n2)cc1)c1cc(Cl)c(NCCCl)c(C(F)(F)F)c1] |
| A69 | 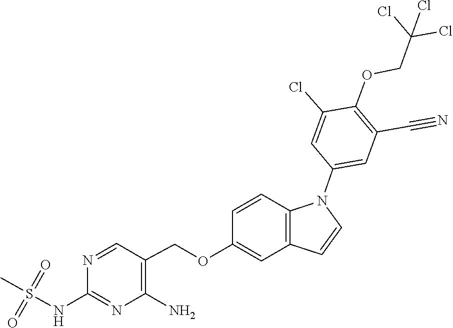 [SMILES: CS(=O)(=O)Nc1ncc(COc2ccc3c(ccn3-c3cc(Cl)c(OCC(Cl)(Cl)Cl)c(C#N)c3)c2)c(N)n1] |
| A70 | 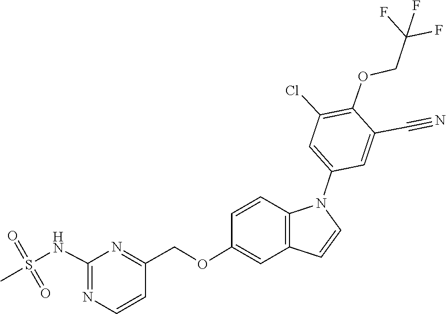 [SMILES: CS(=O)(=O)Nc1nccc(COc2ccc3c(ccn3-c3cc(Cl)c(OCC(F)(F)F)c(C#N)c3)c2)n1] |
| A71 | 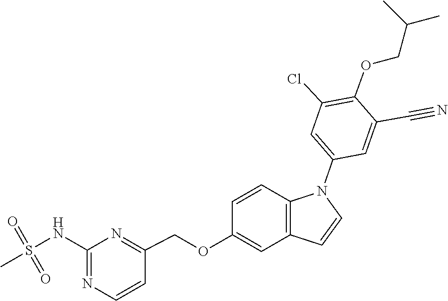 [SMILES: CC(C)COc1c(Cl)cc(-n2ccc3cc(OCc4ccnc(NS(C)(=O)=O)n4)ccc32)cc1C#N] |
| A72 | 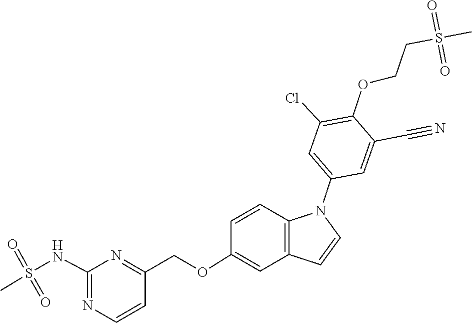 |
| A73 | 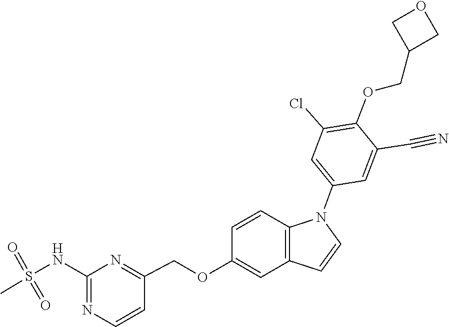 [SMILES: CS(=O)(=O)Nc1nccc(COc2ccc3c(ccn3-c3cc(Cl)c(OCC4COC4)c(C#N)c3)c2)n1] |
| A74 | 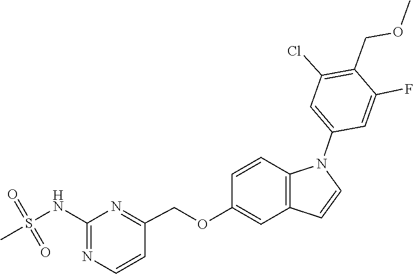 |
| A75 | 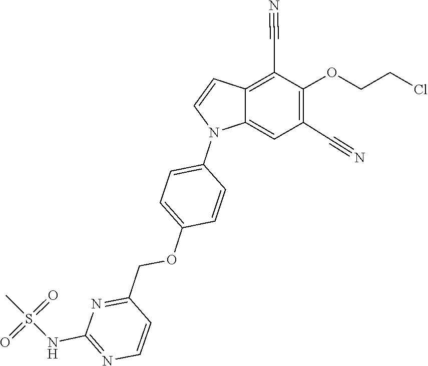 [SMILES: CS(=O)(=O)Nc1nccc(COc2ccc(-n3ccc4c(C#N)c(OCCCl)c(C#N)cc43)cc2)n1] |
| A76 | 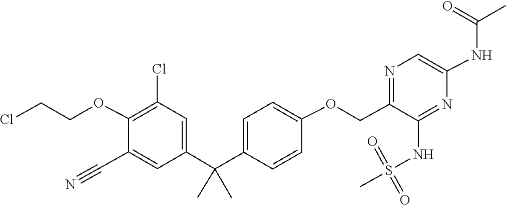 [SMILES: CC(=O)Nc1cnc(COc2ccc(C(C)(C)c3cc(Cl)c(OCCCl)c(C#N)c3)cc2)c(NS(C)(=O)=O)n1] |
| A77 | 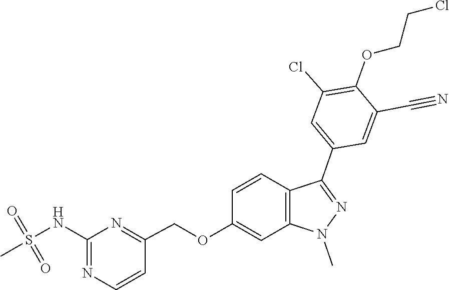 [SMILES: Cn1nc(-c2cc(Cl)c(OCCCl)c(C#N)c2)c2ccc(OCc3ccnc(NS(C)(=O)=O)n3)cc21] |
| A78 | 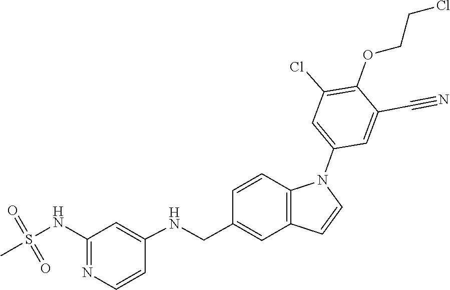 [SMILES: CS(=O)(=O)Nc1cc(NCc2ccc3c(ccn3-c3cc(Cl)c(OCCCl)c(C#N)c3)c2)ccn1] |
| A79 | 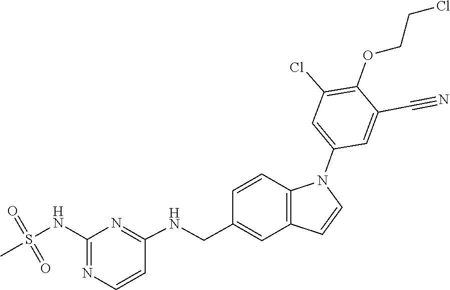 [SMILES: CS(=O)(=O)Nc1nccc(NCc2ccc3c(ccn3-c3cc(Cl)c(OCCCl)c(C#N)c3)c2)n1] |
| A80 | 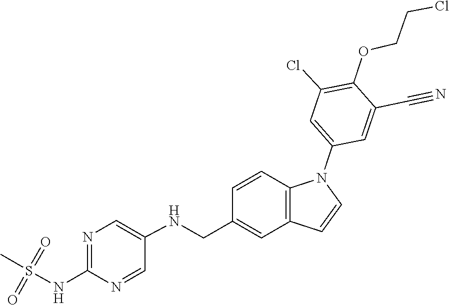 [SMILES: CS(=O)(=O)Nc1ncc(NCc2ccc3c(ccn3-c3cc(Cl)c(OCCCl)c(C#N)c3)c2)cn1] |
| A81 | 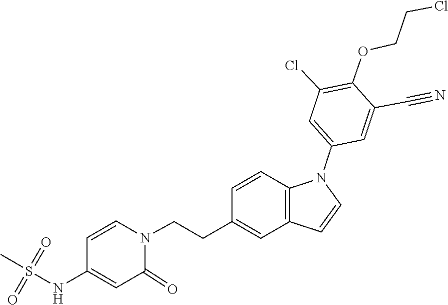 [SMILES: CS(=O)(=O)Nc1ccn(CCc2ccc3c(ccn3-c3cc(Cl)c(OCCCl)c(C#N)c3)c2)c(=O)c1] |
| A82 | 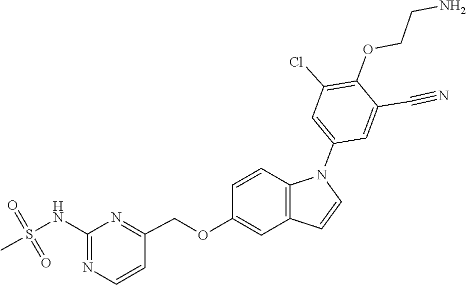 [SMILES: CS(=O)(=O)Nc1nccc(COc2ccc3c(ccn3-c3cc(Cl)c(OCCN)c(C#N)c3)c2)n1] |
| A83 | 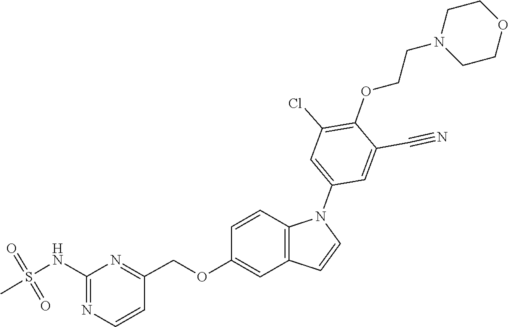 [SMILES: CS(=O)(=O)Nc1nccc(COc2ccc3c(ccn3-c3cc(Cl)c(OCCN4CCOCC4)c(C#N)c3)c2)n1] |
| A84 | 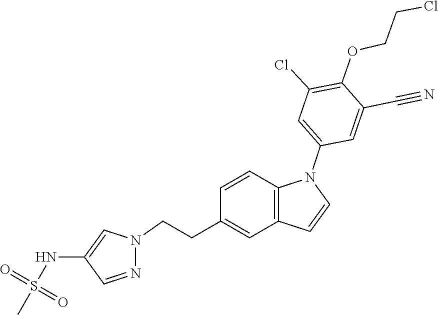 [SMILES: CS(=O)(=O)Nc1cnn(CCc2ccc3c(ccn3-c3cc(Cl)c(OCCCl)c(C#N)c3)c2)c1] |
| A85 | 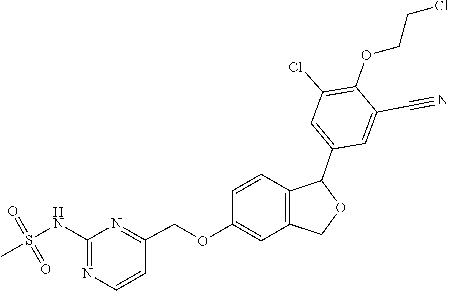 [SMILES: CS(=O)(=O)Nc1nccc(COc2ccc3c(c2)COC3c2cc(Cl)c(OCCCl)c(C#N)c2)n1] |
| A86 | 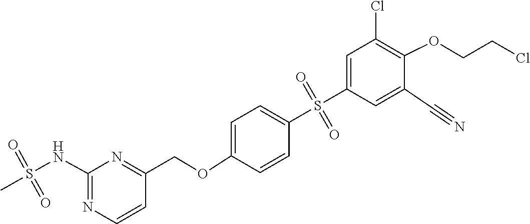 [SMILES: CS(=O)(=O)Nc1nccc(COc2ccc(S(=O)(=O)c3cc(Cl)c(OCCCl)c(C#N)c3)cc2)n1] |
| A87 | 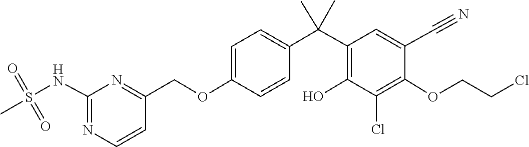 [SMILES: CC(C)(c1ccc(OCc2ccnc(NS(C)(=O)=O)n2)cc1)c1cc(C#N)c(OCCCl)c(Cl)c1O] |
| A88 | 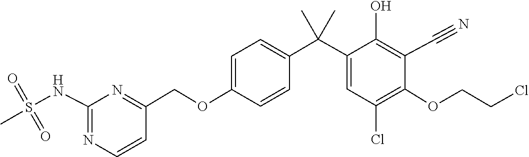 [SMILES: CC(C)(c1ccc(OCc2ccnc(NS(C)(=O)=O)n2)cc1)c1cc(Cl)c(OCCCl)c(C#N)c1O] |
| A89 | 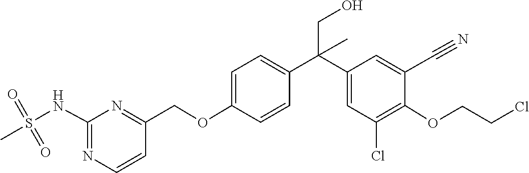 [SMILES: CC(CO)(c1ccc(OCc2ccnc(NS(C)(=O)=O)n2)cc1)c1cc(Cl)c(OCCCl)c(C#N)c1] |
| A90 | 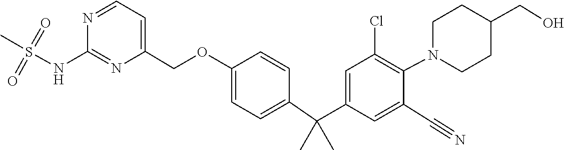 [SMILES: CC(C)(c1ccc(OCc2ccnc(NS(C)(=O)=O)n2)cc1)c1cc(Cl)c(N2CCC(CO)CC2)c(C#N)c1] |
| A91 | 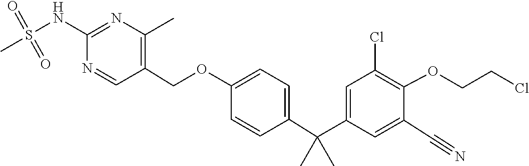 [SMILES: Cc1nc(NS(C)(=O)=O)ncc1COc1ccc(C(C)(C)c2cc(Cl)c(OCCCl)c(C#N)c2)cc1] |
| A92 | 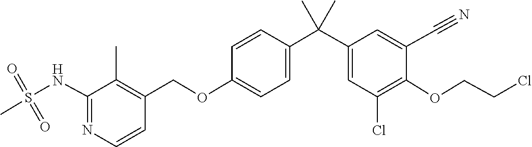 [SMILES: Cc1c(COc2ccc(C(C)(C)c3cc(Cl)c(OCCCl)c(C#N)c3)cc2)ccnc1NS(C)(=O)=O] |
| A93 | 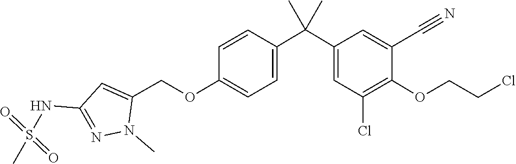 [SMILES: Cn1nc(NS(C)(=O)=O)cc1COc1ccc(C(C)(C)c2cc(Cl)c(OCCCl)c(C#N)c2)cc1] |
| A94 | 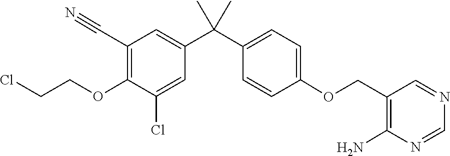 [SMILES: CC(C)(c1ccc(OCc2cncnc2N)cc1)c1cc(Cl)c(OCCCl)c(C#N)c1] |
| A95 | 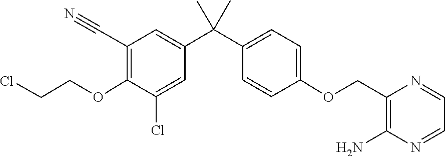 [SMILES: CC(C)(c1ccc(OCc2nccnc2N)cc1)c1cc(Cl)c(OCCCl)c(C#N)c1] |
| A96 | 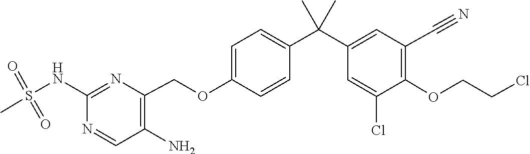 [SMILES: CC(C)(c1ccc(OCc2nc(NS(C)(=O)=O)ncc2N)cc1)c1cc(Cl)c(OCCCl)c(C#N)c1] |
| A97 | 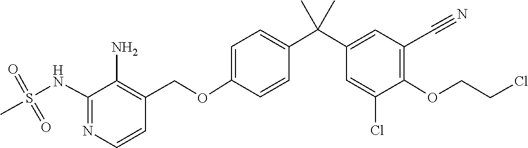 [SMILES: CC(C)(c1ccc(OCc2ccnc(NS(C)(=O)=O)c2N)cc1)c1cc(Cl)c(OCCCl)c(C#N)c1] |
| A98 | 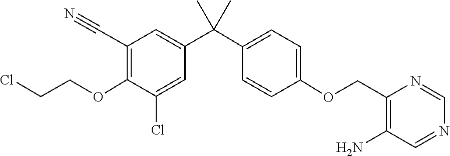 [SMILES: CC(C)(c1ccc(OCc2ncncc2N)cc1)c1cc(Cl)c(OCCCl)c(C#N)c1] |
| A99 | 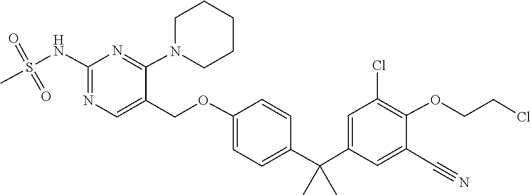 [SMILES: CC(C)(c1ccc(OCc2cnc(NS(C)(=O)=O)nc2N2CCCCC2)cc1)c1cc(Cl)c(OCCCl)c(C#N)c1] |
| A100 | 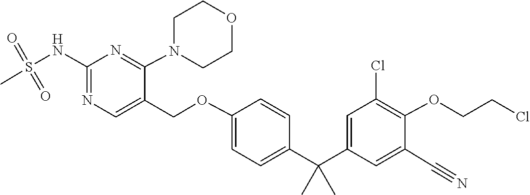 [SMILES: CC(C)(c1ccc(OCc2cnc(NS(C)(=O)=O)nc2N2CCOCC2)cc1)c1cc(Cl)c(OCCCl)c(C#N)c1] |
| A101 | 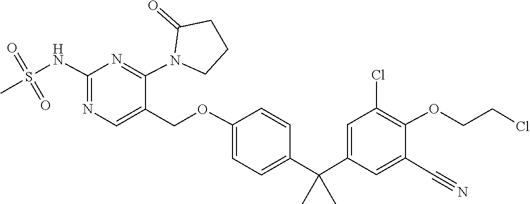 [SMILES: CC(C)(c1ccc(OCc2cnc(NS(C)(=O)=O)nc2N2CCCC2=O)cc1)c1cc(Cl)c(OCCCl)c(C#N)c1] |
| A102 | 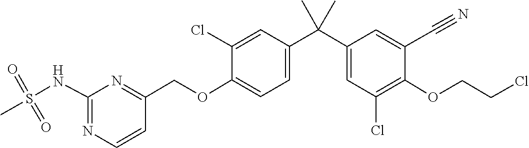 [SMILES: CC(C)(c1ccc(OCc2ccnc(NS(C)(=O)=O)n2)c(Cl)c1)c1cc(Cl)c(OCCCl)c(C#N)c1] |
| A103 | 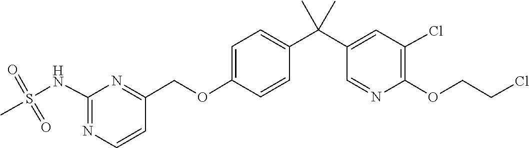 [SMILES: CC(C)(c1ccc(OCc2ccnc(NS(C)(=O)=O)n2)cc1)c1cnc(OCCCl)c(Cl)c1] |
| A104 | 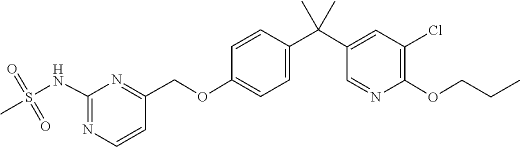 [SMILES: CCCOc1ncc(C(C)(C)c2ccc(OCc3ccnc(NS(C)(=O)=O)n3)cc2)cc1Cl] |
| A105 | 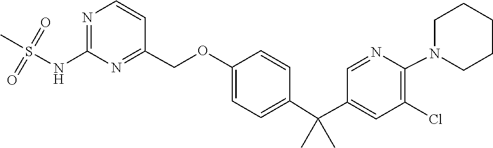 [SMILES: CC(C)(c1ccc(OCc2ccnc(NS(C)(=O)=O)n2)cc1)c1cnc(N2CCCCC2)c(Cl)c1] |
| A106 | 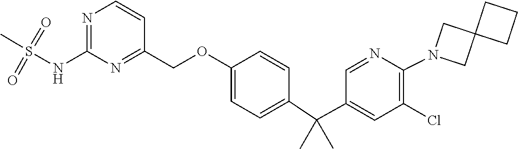 [SMILES: CC(C)(c1ccc(OCc2ccnc(NS(C)(=O)=O)n2)cc1)c1cnc(N2CC3(CCC3)C2)c(Cl)c1] |
| A107 | 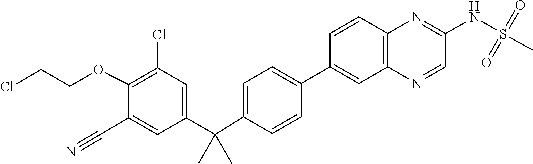 [SMILES: CC(C)(c1ccc(-c2ccc3nc(NS(C)(=O)=O)cnc3c2)cc1)c1cc(Cl)c(OCCCl)c(C#N)c1] |
| A108 | 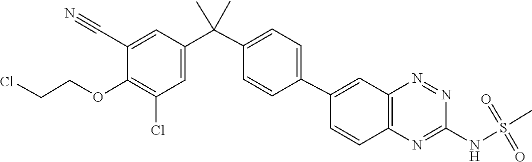 [SMILES: CC(C)(c1ccc(-c2ccc3nc(NS(C)(=O)=O)nnc3c2)cc1)c1cc(Cl)c(OCCCl)c(C#N)c1] |
| A109 | 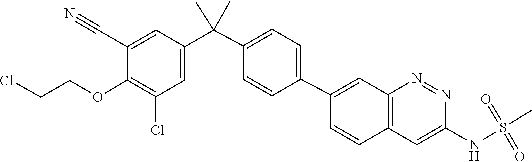 [SMILES: CC(C)(c1ccc(-c2ccc3cc(NS(C)(=O)=O)nnc3c2)cc1)c1cc(Cl)c(OCCCl)c(C#N)c1] |
| A110 | 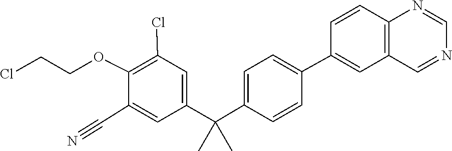 [SMILES: CC(C)(c1ccc(-c2ccc3ncncc3c2)cc1)c1cc(Cl)c(OCCCl)c(C#N)c1] |
| A111 | 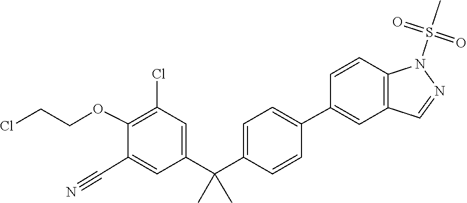 [SMILES: CC(C)(c1ccc(-c2ccc3c(cnn3S(C)(=O)=O)c2)cc1)c1cc(Cl)c(OCCCl)c(C#N)c1] |
| A112 | 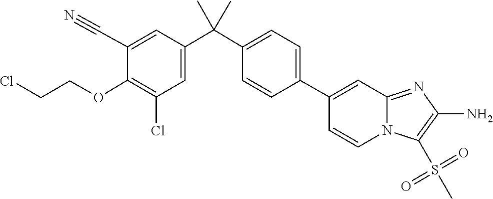 [SMILES: CC(C)(c1ccc(-c2ccn3c(S(C)(=O)=O)c(N)nc3c2)cc1)c1cc(Cl)c(OCCCl)c(C#N)c1] |
| A113 | 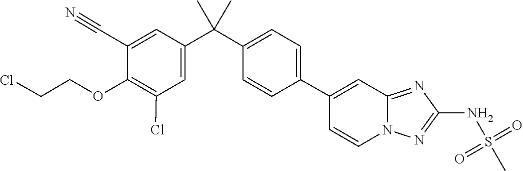 [SMILES: CC(C)(c1ccc(-c2ccn3nc(NS(C)(=O)=O)nc3c2)cc1)c1cc(Cl)c(OCCCl)c(C#N)c1] |
| A114 | 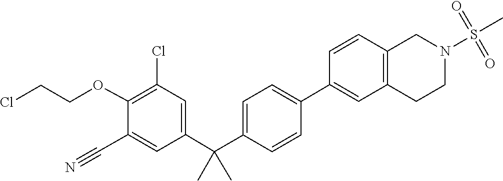 [SMILES: CC(C)(c1ccc(-c2ccc3c(c2)CCN(S(C)(=O)=O)C3)cc1)c1cc(Cl)c(OCCCl)c(C#N)c1] |
| A115 | 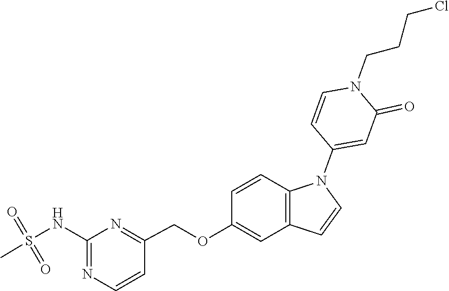 [SMILES: CS(=O)(=O)Nc1nccc(COc2ccc3c(ccn3-c3ccn(CCCCl)c(=O)c3)c2)n1] |
| A116 | 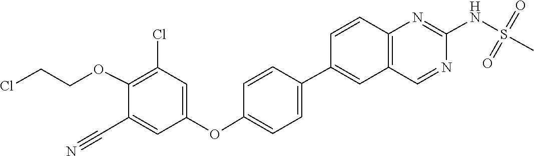 [SMILES: CS(=O)(=O)Nc1ncc2cc(-c3ccc(Oc4cc(Cl)c(OCCCl)c(C#N)c4)cc3)ccc2n1] |
| A117 | 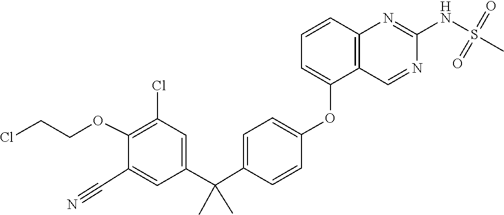 [SMILES: CC(C)(c1ccc(Oc2cccc3nc(NS(C)(=O)=O)ncc23)cc1)c1cc(Cl)c(OCCCl)c(C#N)c1] |
| A118 | 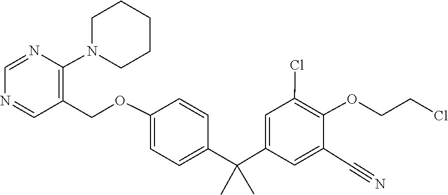 [SMILES: CC(C)(c1ccc(OCc2cncnc2N2CCCCC2)cc1)c1cc(Cl)c(OCCCl)c(C#N)c1] |
| A119 | 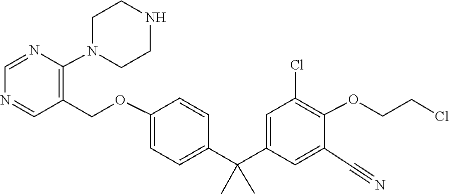 [SMILES: CC(C)(c1ccc(OCc2cncnc2N2CCNCC2)cc1)c1cc(Cl)c(OCCCl)c(C#N)c1] |
| A120 | 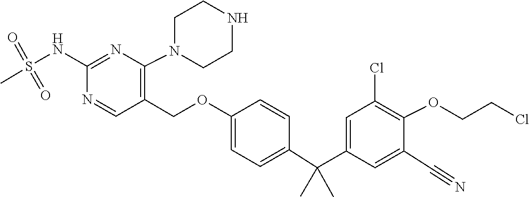 [SMILES: CC(C)(c1ccc(OCc2cnc(NS(C)(=O)=O)nc2N2CCNCC2)cc1)c1cc(Cl)c(OCCCl)c(C#N)c1] |
| A121 | 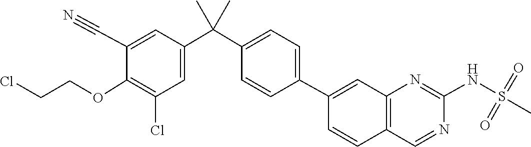 [SMILES: CC(C)(c1ccc(-c2ccc3cnc(NS(C)(=O)=O)nc3c2)cc1)c1cc(Cl)c(OCCCl)c(C#N)c1] |
| A122 | 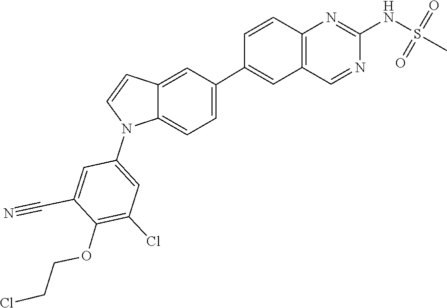 [SMILES: CS(=O)(=O)Nc1ncc2cc(-c3ccc4c(ccn4-c4cc(Cl)c(OCCCl)c(C#N)c4)c3)ccc2n1] |
| A123 | 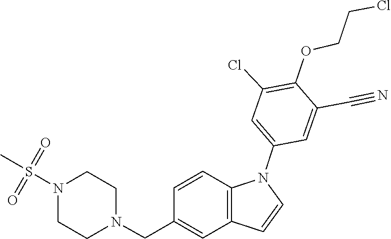 [SMILES: CS(=O)(=O)N1CCN(Cc2ccc3c(ccn3-c3cc(Cl)c(OCCCl)c(C#N)c3)c2)CC1] |
| A124 | 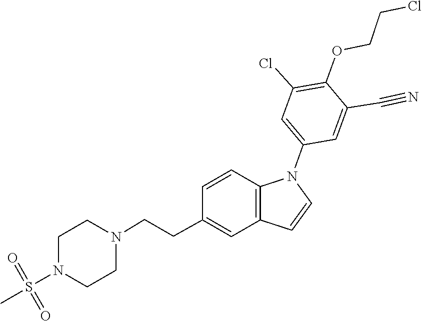 [SMILES: CS(=O)(=O)N1CCN(CCc2ccc3c(ccn3-c3cc(Cl)c(OCCCl)c(C#N)c3)c2)CC1] |
| A125 | 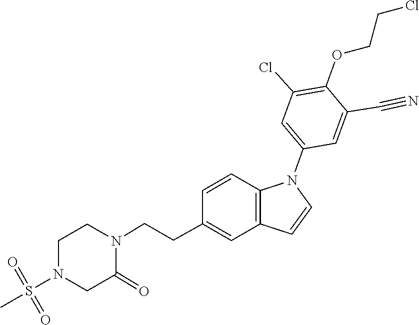 [SMILES: CS(=O)(=O)N1CCN(CCc2ccc3c(ccn3-c3cc(Cl)c(OCCCl)c(C#N)c3)c2)C(=O)C1] |
| A126 | 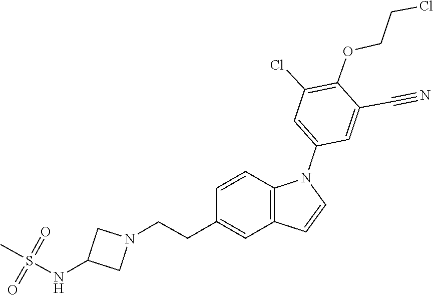 [SMILES: CS(=O)(=O)NC1CN(CCc2ccc3c(ccn3-c3cc(Cl)c(OCCCl)c(C#N)c3)c2)C1] |
| A127 | 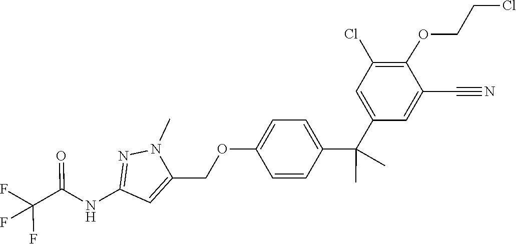 [SMILES: Cn1nc(NC(=O)C(F)(F)F)cc1COc1ccc(C(C)(C)c2cc(Cl)c(OCCCl)c(C#N)c2)cc1] |
| A128 | 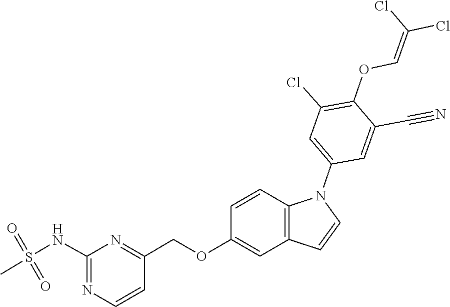 [SMILES: CS(=O)(=O)Nc1nccc(COc2ccc3c(ccn3-c3cc(Cl)c(OC=C(Cl)Cl)c(C#N)c3)c2)n1] |
| A129 | 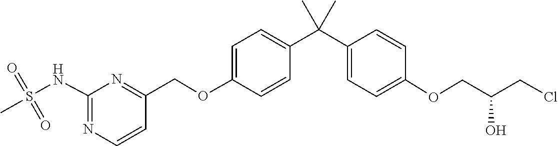 [SMILES: CC(C)(c1ccc(OCc2ccnc(NS(C)(=O)=O)n2)cc1)c1ccc(OC[C@@H](O)CCl)cc1] |
| A130 | 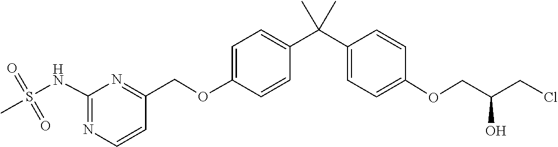 [SMILES: CC(C)(c1ccc(OCc2ccnc(NS(C)(=O)=O)n2)cc1)c1ccc(OC[C@H](O)CCl)cc1] |
| A131 | 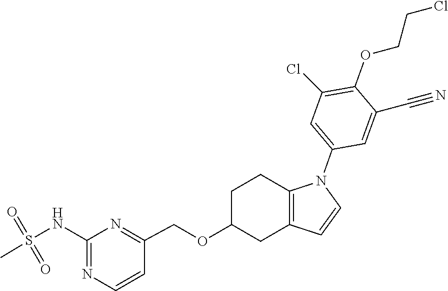 [SMILES: CS(=O)(=O)Nc1nccc(COC2CCc3c(ccn3-c3cc(Cl)c(OCCCl)c(C#N)c3)C2)n1] |
| A132 | 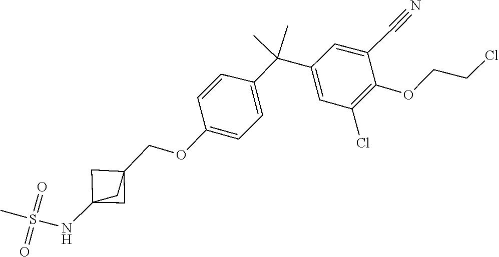 [SMILES: CC(C)(c1ccc(OCC23CC(NS(C)(=O)=O)(C2)C3)cc1)c1cc(Cl)c(OCCCl)c(C#N)c1] |
| A133 | 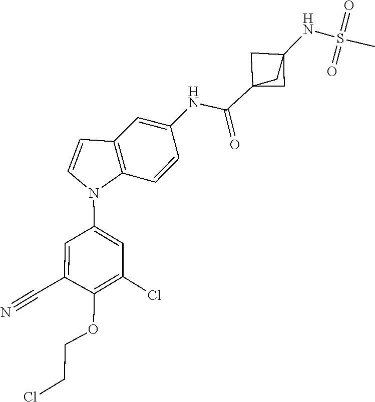 [SMILES: CS(=O)(=O)NC12CC(C(=O)Nc3ccc4c(ccn4-c4cc(Cl)c(OCCCl)c(C#N)c4)c3)(C1)C2] |
| A134 | 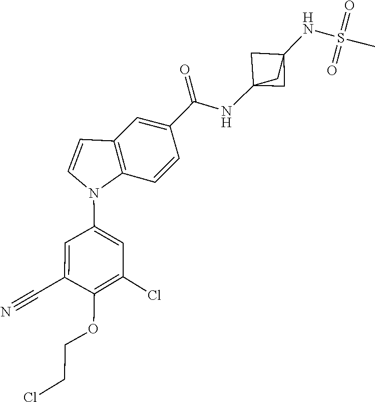 [SMILES: CS(=O)(=O)NC12CC(NC(=O)c3ccc4c(ccn4-c4cc(Cl)c(OCCCl)c(C#N)c4)c3)(C1)C2] |
| A135 | 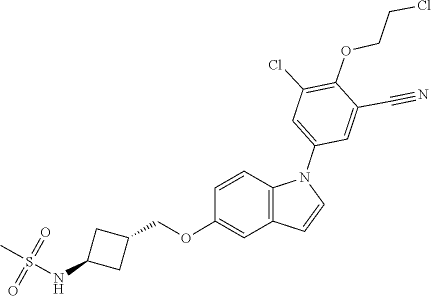 [SMILES: CS(=O)(=O)N[C@H]1C[C@H](COc2ccc3c(ccn3-c3cc(Cl)c(OCCCl)c(C#N)c3)c2)C1] |
| A136 | 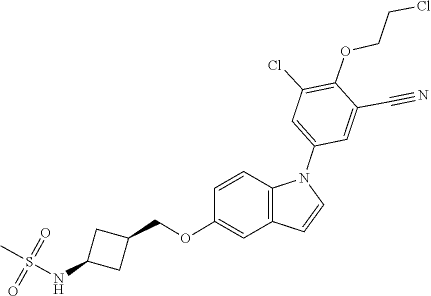 [SMILES: CS(=O)(=O)N[C@H]1C[C@@H](COc2ccc3c(ccn3-c3cc(Cl)c(OCCCl)c(C#N)c3)c2)C1] |
| A137 | 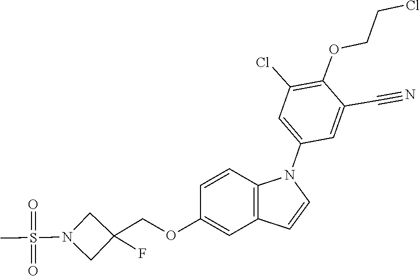 [SMILES: CS(=O)(=O)N1CC(F)(COc2ccc3c(ccn3-c3cc(Cl)c(OCCCl)c(C#N)c3)c2)C1] |
| A138 | 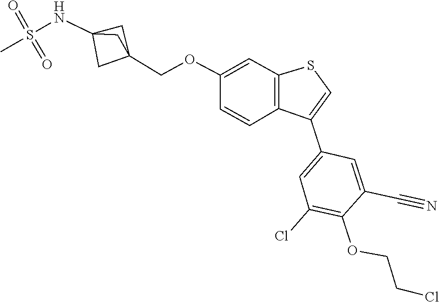 [SMILES: CS(=O)(=O)NC12CC(COc3ccc4c(-c5cc(Cl)c(OCCCl)c(C#N)c5)csc4c3)(C1)C2] |
| A139 | 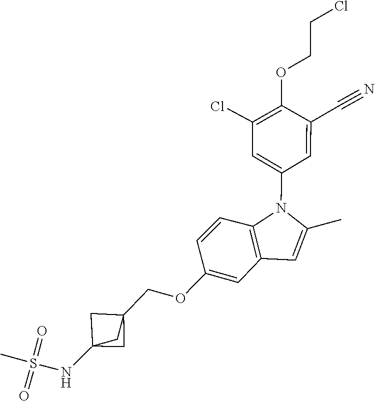 [SMILES: Cc1cc2cc(OCC34CC(NS(C)(=O)=O)(C3)C4)ccc2n1-c1cc(Cl)c(OCCCl)c(C#N)c1] |
| A140 | 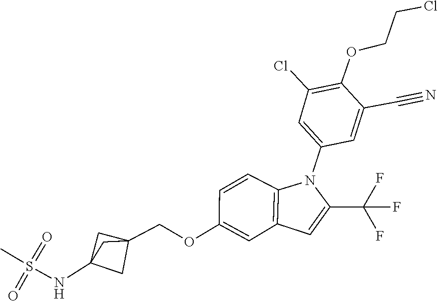 [SMILES: CS(=O)(=O)NC12CC(COc3ccc4c(c3)cc(C(F)(F)F)n4-c3cc(Cl)c(OCCCl)c(C#N)c3)(C1)C2] |
| A141 | 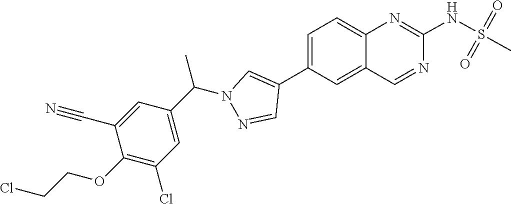 [SMILES: CC(c1cc(Cl)c(OCCCl)c(C#N)c1)n1cc(-c2ccc3nc(NS(C)(=O)=O)ncc3c2)cn1] |
| A142 | 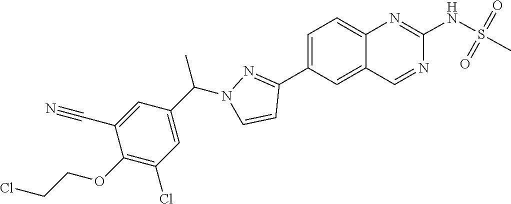 [SMILES: CC(c1cc(Cl)c(OCCCl)c(C#N)c1)n1ccc(-c2ccc3nc(NS(C)(=O)=O)ncc3c2)n1] |
| A143 | 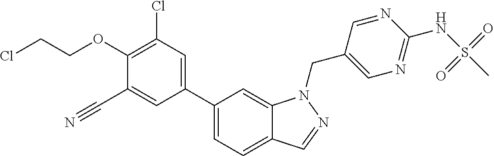 [SMILES: CS(=O)(=O)Nc1ncc(Cn2ncc3ccc(-c4cc(Cl)c(OCCCl)c(C#N)c4)cc32)cn1] |
| A144 | 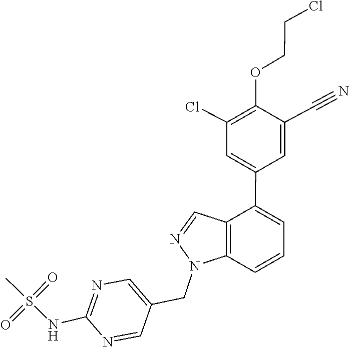 [SMILES: CS(=O)(=O)Nc1ncc(Cn2ncc3c(-c4cc(Cl)c(OCCCl)c(C#N)c4)cccc32)cn1] |
| A145 | 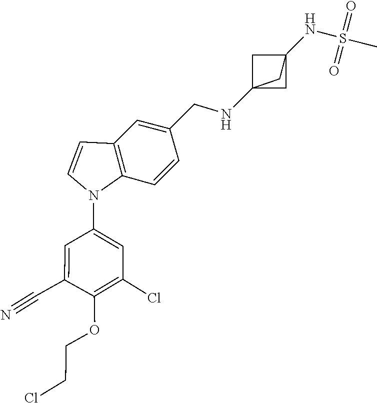 [SMILES: CS(=O)(=O)NC12CC(NCc3ccc4c(ccn4-c4cc(Cl)c(OCCCl)c(C#N)c4)c3)(C1)C2] |
| A146 | 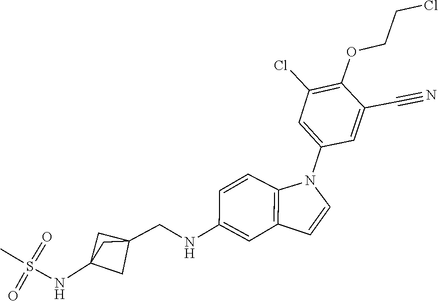 [SMILES: CS(=O)(=O)NC12CC(CNc3ccc4c(ccn4-c4cc(Cl)c(OCCCl)c(C#N)c4)c3)(C1)C2] |
| A147 | 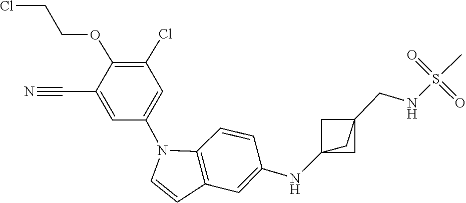 [SMILES: CS(=O)(=O)NCC12CC(Nc3ccc4c(ccn4-c4cc(Cl)c(OCCCl)c(C#N)c4)c3)(C1)C2] |
| A148 | 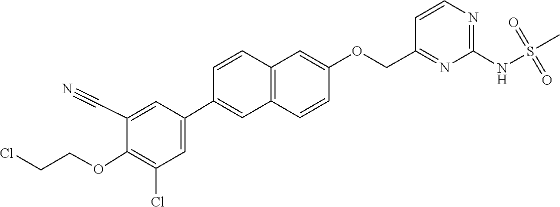 [SMILES: CS(=O)(=O)Nc1nccc(COc2ccc3cc(-c4cc(Cl)c(OCCCl)c(C#N)c4)ccc3c2)n1] |
| A149 | 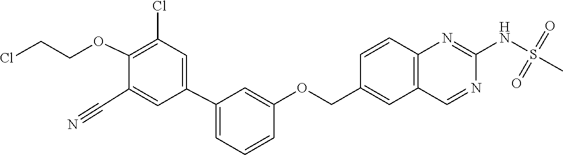 [SMILES: CS(=O)(=O)Nc1ncc2cc(COc3cccc(-c4cc(Cl)c(OCCCl)c(C#N)c4)c3)ccc2n1] |
| A150 | 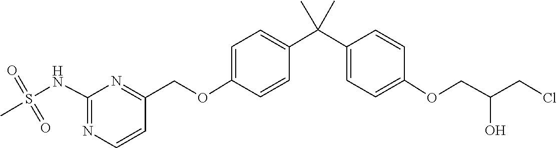 [SMILES: CC(C)(c1ccc(OCc2ccnc(NS(C)(=O)=O)n2)cc1)c1ccc(OCC(O)CCl)cc1] |
| A151 | 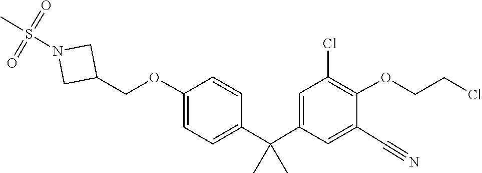 [SMILES: CC(C)(c1ccc(OCC2CN(S(C)(=O)=O)C2)cc1)c1cc(Cl)c(OCCCl)c(C#N)c1] |
| A152 | 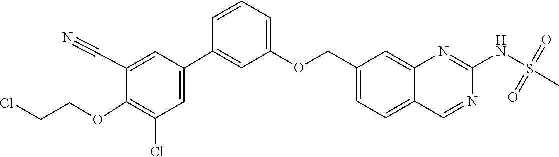 [SMILES: CS(=O)(=O)Nc1ncc2ccc(COc3cccc(-c4cc(Cl)c(OCCCl)c(C#N)c4)c3)cc2n1] |
| A153 | 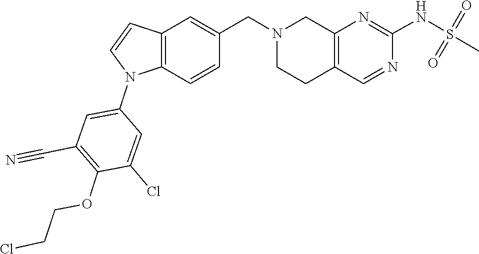 [SMILES: CS(=O)(=O)Nc1ncc2c(n1)CN(Cc1ccc3c(ccn3-c3cc(Cl)c(OCCCl)c(C#N)c3)c1)CC2] |
| A154 | 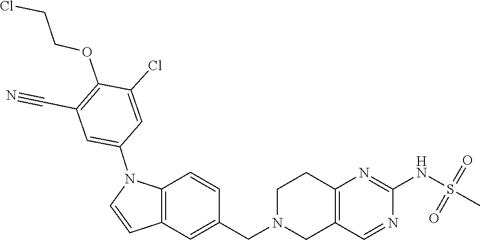 [SMILES: CS(=O)(=O)Nc1ncc2c(n1)CCN(Cc1ccc3c(ccn3-c3cc(Cl)c(OCCCl)c(C#N)c3)c1)C2] |
| A155 | 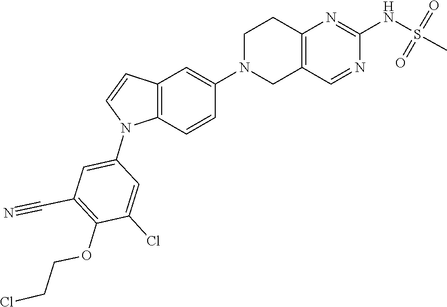 [SMILES: CS(=O)(=O)Nc1ncc2c(n1)CCN(c1ccc3c(ccn3-c3cc(Cl)c(OCCCl)c(C#N)c3)c1)C2] |
| A156 | 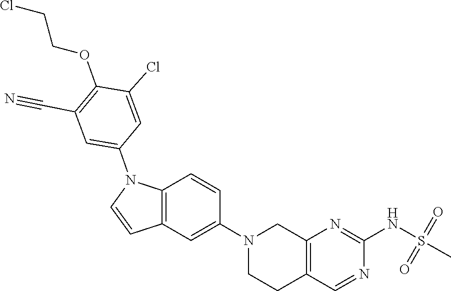 [SMILES: CS(=O)(=O)Nc1ncc2c(n1)CN(c1ccc3c(ccn3-c3cc(Cl)c(OCCCl)c(C#N)c3)c1)CC2] |
| A157 | 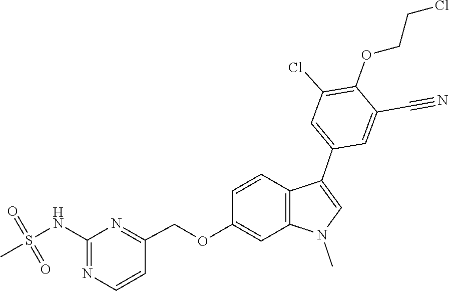 [SMILES: Cn1cc(-c2cc(Cl)c(OCCCl)c(C#N)c2)c2ccc(OCc3ccnc(NS(C)(=O)=O)n3)cc21] |
| A158 | 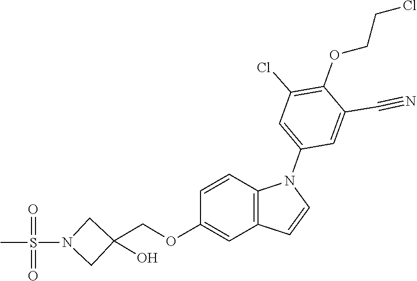 [SMILES: CS(=O)(=O)N1CC(O)(COc2ccc3c(ccn3-c3cc(Cl)c(OCCCl)c(C#N)c3)c2)C1] |
| A159 | 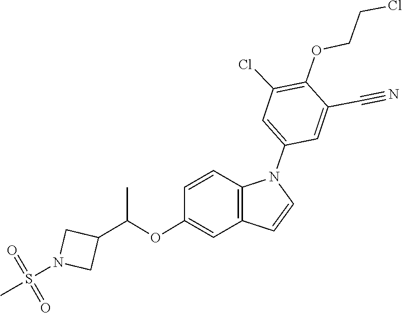 [SMILES: CC(Oc1ccc2c(ccn2-c2cc(Cl)c(OCCCl)c(C#N)c2)c1)C1CN(S(C)(=O)=O)C1] |
| A160 | 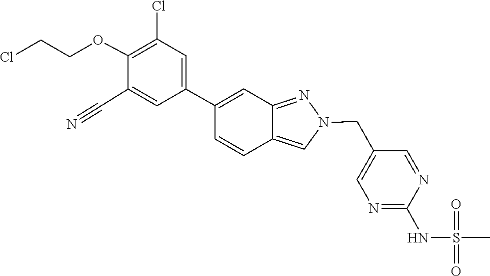 [SMILES: CS(=O)(=O)Nc1ncc(Cn2cc3ccc(-c4cc(Cl)c(OCCCl)c(C#N)c4)cc3n2)cn1] |
| A161 | 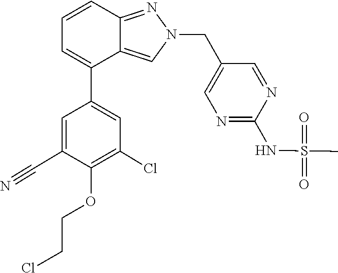 [SMILES: CS(=O)(=O)Nc1ncc(Cn2cc3c(-c4cc(Cl)c(OCCCl)c(C#N)c4)cccc3n2)cn1] |
| A162 | 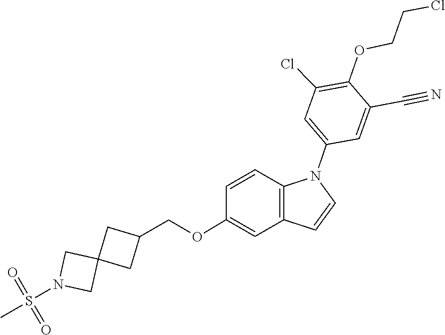 [SMILES: CS(=O)(=O)N1CC2(CC(COc3ccc4c(ccn4-c4cc(Cl)c(OCCCl)c(C#N)c4)c3)C2)C1] |
| A163 | 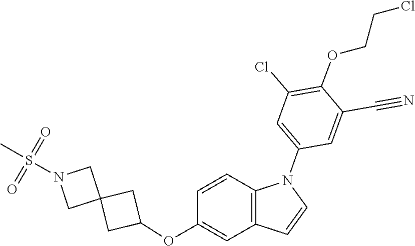 [SMILES: CS(=O)(=O)N1CC2(CC(Oc3ccc4c(ccn4-c4cc(Cl)c(OCCCl)c(C#N)c4)c3)C2)C1] |
| A164 | 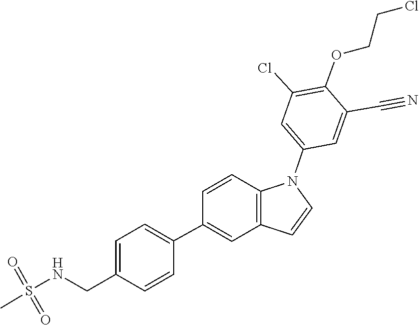 [SMILES: CS(=O)(=O)NCc1ccc(-c2ccc3c(ccn3-c3cc(Cl)c(OCCCl)c(C#N)c3)c2)cc1] |
| A165 | 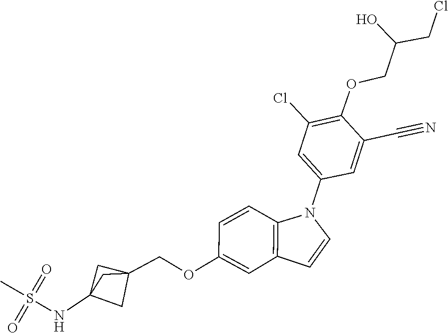 [SMILES: CS(=O)(=O)NC12CC(COc3ccc4c(ccn4-c4cc(Cl)c(OCC(O)CCl)c(C#N)c4)c3)(C1)C2] |
| A166 | 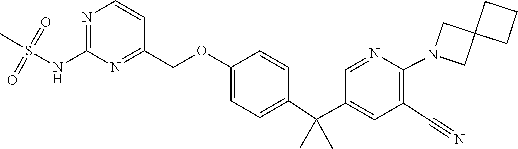 [SMILES: CC(C)(c1ccc(OCc2ccnc(NS(C)(=O)=O)n2)cc1)c1cnc(N2CC3(CCC3)C2)c(C#N)c1] |
| A167 | 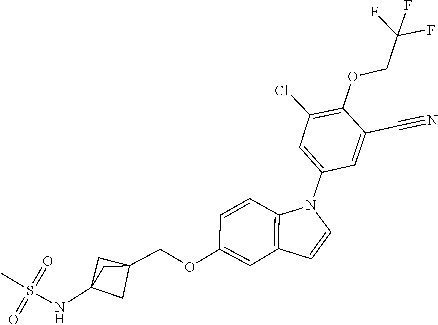 [SMILES: CS(=O)(=O)NC12CC(COc3ccc4c(ccn4-c4cc(Cl)c(OCC(F)(F)F)c(C#N)c4)c3)(C1)C2] |
| A168 | 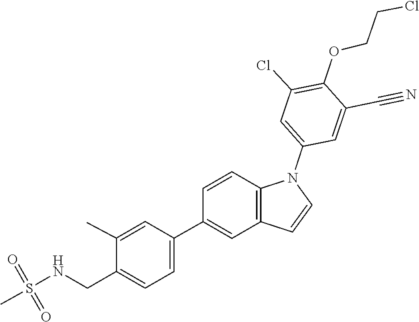 [SMILES: Cc1cc(-c2ccc3c(ccn3-c3cc(Cl)c(OCCCl)c(C#N)c3)c2)ccc1CNS(C)(=O)=O] |
| A169 | 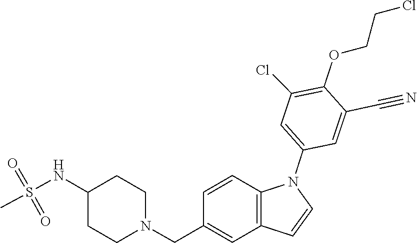 [SMILES: CS(=O)(=O)NC1CCN(Cc2ccc3c(ccn3-c3cc(Cl)c(OCCCl)c(C#N)c3)c2)CC1] |
| A170 | 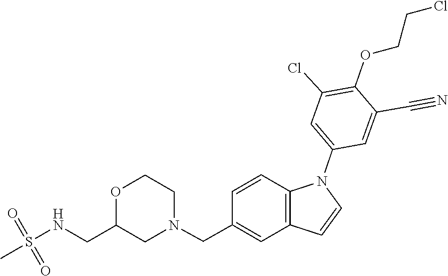 [SMILES: CS(=O)(=O)NCC1CN(Cc2ccc3c(ccn3-c3cc(Cl)c(OCCCl)c(C#N)c3)c2)CCO1] |
| A171 | 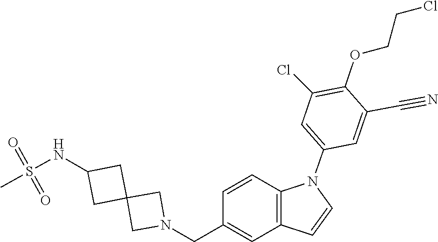 [SMILES: CS(=O)(=O)NC1CC2(C1)CN(Cc1ccc3c(ccn3-c3cc(Cl)c(OCCCl)c(C#N)c3)c1)C2] |
| A172 | 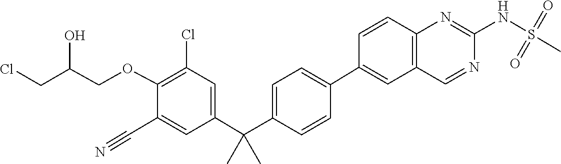 [SMILES: CC(C)(c1ccc(-c2ccc3nc(NS(C)(=O)=O)ncc3c2)cc1)c1cc(Cl)c(OCC(O)CCl)c(C#N)c1] |
| A173 | 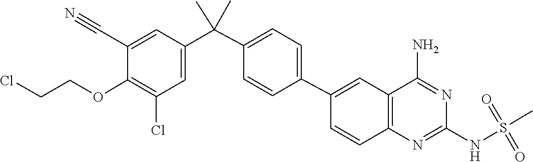 [SMILES: CC(C)(c1ccc(-c2ccc3nc(NS(C)(=O)=O)nc(N)c3c2)cc1)c1cc(Cl)c(OCCCl)c(C#N)c1] |
| A174 | 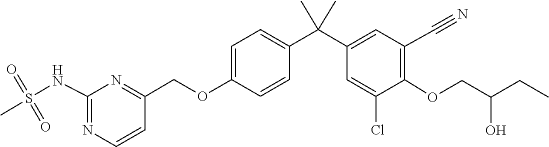 [SMILES: CCC(O)COc1c(Cl)cc(C(C)(C)c2ccc(OCc3ccnc(NS(C)(=O)=O)n3)cc2)cc1C#N] |
| A175 | 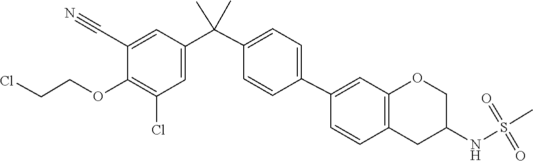 [SMILES: CC(C)(c1ccc(-c2ccc3c(c2)OCC(NS(C)(=O)=O)C3)cc1)c1cc(Cl)c(OCCCl)c(C#N)c1] |
| A176 | 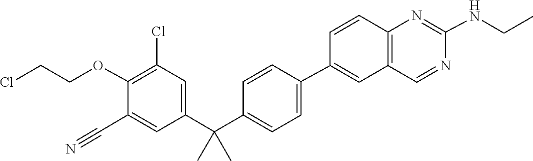 [SMILES: CCNc1ncc2cc(-c3ccc(C(C)(C)c4cc(Cl)c(OCCCl)c(C#N)c4)cc3)ccc2n1] |
| A177 | 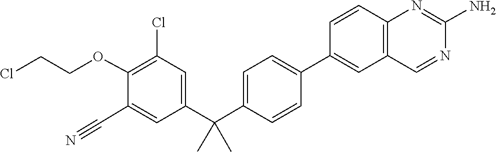 [SMILES: CC(C)(c1ccc(-c2ccc3nc(N)ncc3c2)cc1)c1cc(Cl)c(OCCCl)c(C#N)c1] |
| A178 | 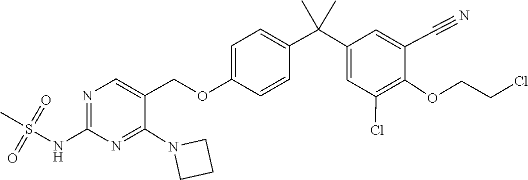 [SMILES: CC(C)(c1ccc(OCc2cnc(NS(C)(=O)=O)nc2N2CCC2)cc1)c1cc(Cl)c(OCCCl)c(C#N)c1] |
| A179 | 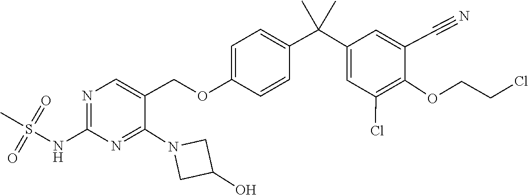 [SMILES: CC(C)(c1ccc(OCc2cnc(NS(C)(=O)=O)nc2N2CC(O)C2)cc1)c1cc(Cl)c(OCCCl)c(C#N)c1] |
| A180 | 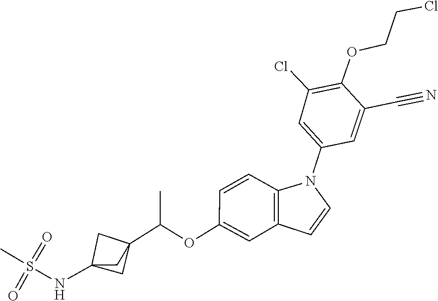 [SMILES: CC(Oc1ccc2c(ccn2-c2cc(Cl)c(OCCCl)c(C#N)c2)c1)C12CC(NS(C)(=O)=O)(C1)C2] |
| A181 | 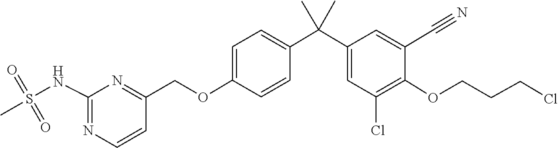 [SMILES: CC(C)(c1ccc(OCc2ccnc(NS(C)(=O)=O)n2)cc1)c1cc(Cl)c(OCCCCl)c(C#N)c1] |
| A182 | 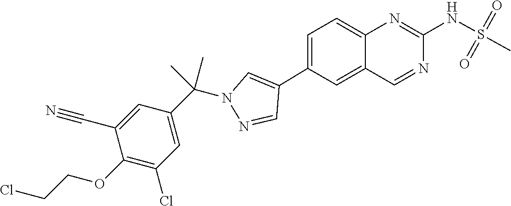 [SMILES: CC(C)(c1cc(Cl)c(OCCCl)c(C#N)c1)n1cc(-c2ccc3nc(NS(C)(=O)=O)ncc3c2)cn1] |
| A183 | 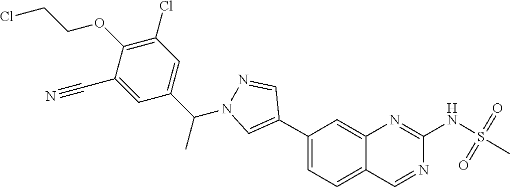 [SMILES: CC(c1cc(Cl)c(OCCCl)c(C#N)c1)n1cc(-c2ccc3cnc(NS(C)(=O)=O)nc3c2)cn1] |
| A184 | 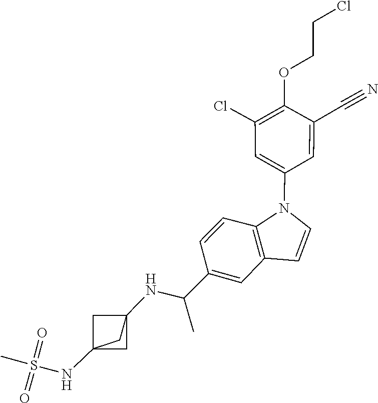 [SMILES: CC(NC12CC(NS(C)(=O)=O)(C1)C2)c1ccc2c(ccn2-c2cc(Cl)c(OCCCl)c(C#N)c2)c1] |
| A185 | 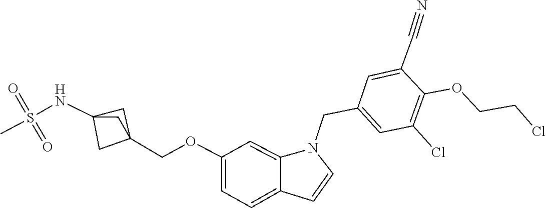 [SMILES: CS(=O)(=O)NC12CC(COc3ccc4ccn(Cc5cc(Cl)c(OCCCl)c(C#N)c5)c4c3)(C1)C2] |
| A186 | 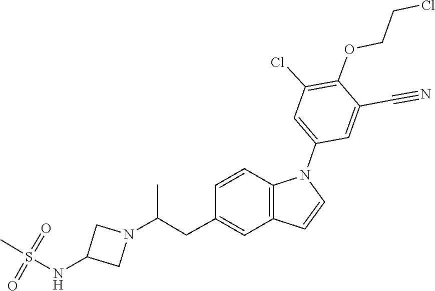 [SMILES: CC(Cc1ccc2c(ccn2-c2cc(Cl)c(OCCCl)c(C#N)c2)c1)N1CC(NS(C)(=O)=O)C1] |
| A187 | 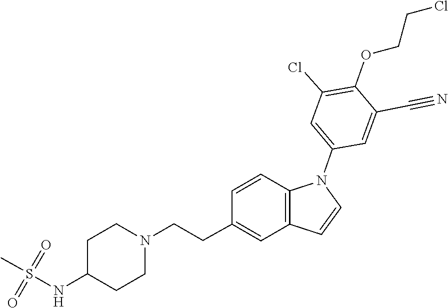 [SMILES: CS(=O)(=O)NC1CCN(CCc2ccc3c(ccn3-c3cc(Cl)c(OCCCl)c(C#N)c3)c2)CC1] |
| A188 | 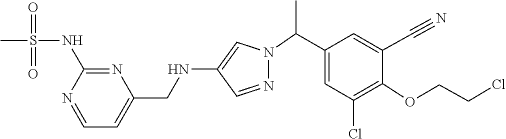 [SMILES: CC(c1cc(Cl)c(OCCCl)c(C#N)c1)n1cc(NCc2ccnc(NS(C)(=O)=O)n2)cn1] |
| A189 | 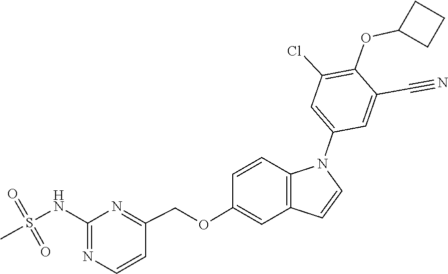 [SMILES: CS(=O)(=O)Nc1nccc(COc2ccc3c(ccn3-c3cc(Cl)c(OC4CCC4)c(C#N)c3)c2)n1] |
| A190 | 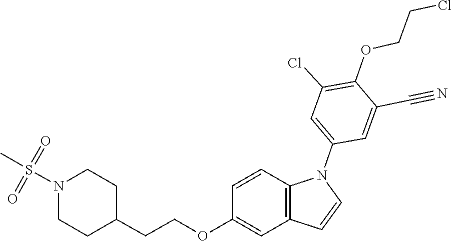 [SMILES: CS(=O)(=O)N1CCC(CCOc2ccc3c(ccn3-c3cc(Cl)c(OCCCl)c(C#N)c3)c2)CC1] |
| A191 | 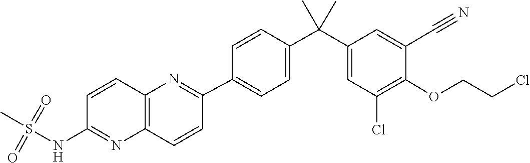 [SMILES: CC(C)(c1ccc(-c2ccc3nc(NS(C)(=O)=O)ccc3n2)cc1)c1cc(Cl)c(OCCCl)c(C#N)c1] |
| A192 | 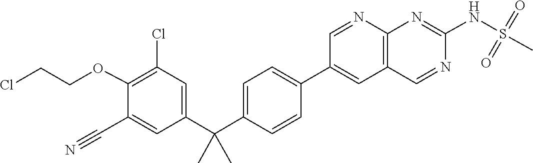 [SMILES: CC(C)(c1ccc(-c2cnc3nc(NS(C)(=O)=O)ncc3c2)cc1)c1cc(Cl)c(OCCCl)c(C#N)c1] |
| A193 | 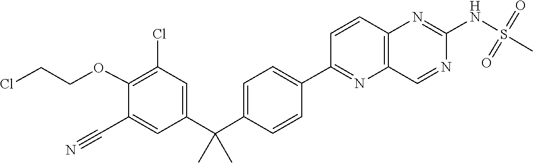 [SMILES: CC(C)(c1ccc(-c2ccc3nc(NS(C)(=O)=O)ncc3n2)cc1)c1cc(Cl)c(OCCCl)c(C#N)c1] |
| A194 | 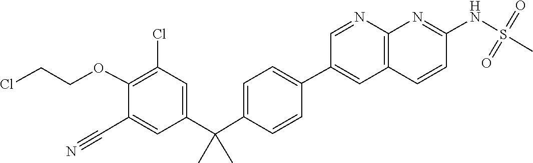 [SMILES: CC(C)(c1ccc(-c2cnc3nc(NS(C)(=O)=O)ccc3c2)cc1)c1cc(Cl)c(OCCCl)c(C#N)c1] |
| A195 | 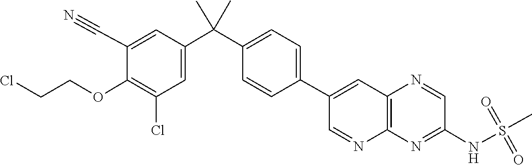 [SMILES: CC(C)(c1ccc(-c2cnc3nc(NS(C)(=O)=O)cnc3c2)cc1)c1cc(Cl)c(OCCCl)c(C#N)c1] |
| A196 | 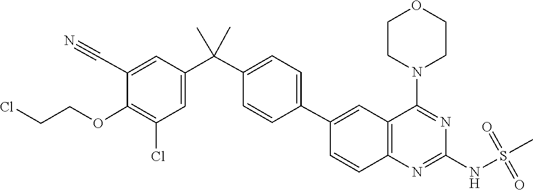 [SMILES: CC(C)(c1ccc(-c2ccc3nc(NS(C)(=O)=O)nc(N4CCOCC4)c3c2)cc1)c1cc(Cl)c(OCCCl)c(C#N)c1] |
| A197 | 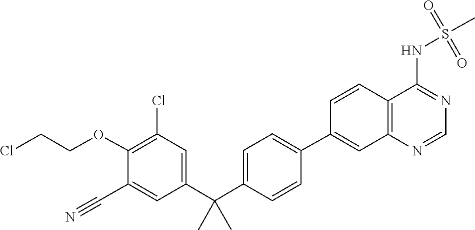 [SMILES: CC(C)(c1ccc(-c2ccc3c(NS(C)(=O)=O)ncnc3c2)cc1)c1cc(Cl)c(OCCCl)c(C#N)c1] |
| A198 | 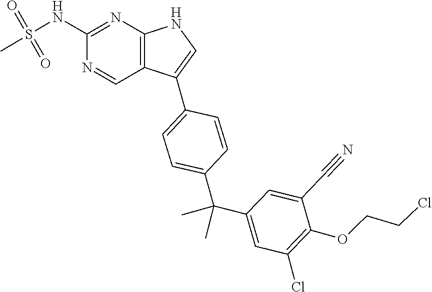 [SMILES: CC(C)(c1ccc(-c2c[nH]c3nc(NS(C)(=O)=O)ncc23)cc1)c1cc(Cl)c(OCCCl)c(C#N)c1] |
| A199 | 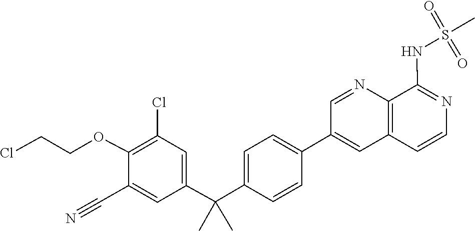 [SMILES: CC(C)(c1ccc(-c2cnc3c(NS(C)(=O)=O)nccc3c2)cc1)c1cc(Cl)c(OCCCl)c(C#N)c1] |
| A200 | 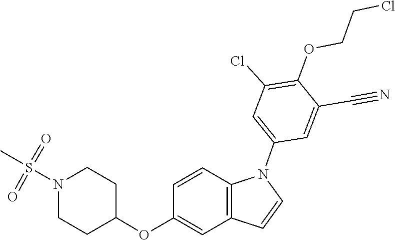 [SMILES: CS(=O)(=O)N1CCC(Oc2ccc3c(ccn3-c3cc(Cl)c(OCCCl)c(C#N)c3)c2)CC1] |
| A201 | 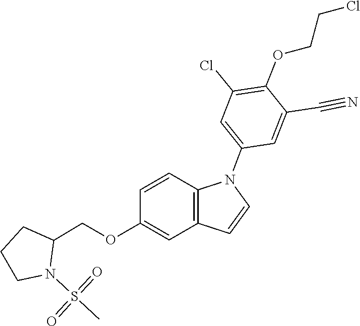 [SMILES: CS(=O)(=O)N1CCCC1COc1ccc2c(ccn2-c2cc(Cl)c(OCCCl)c(C#N)c2)c1] |
| A202 | 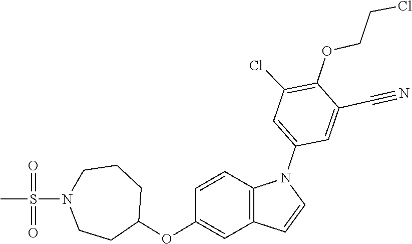 [SMILES: CS(=O)(=O)N1CCCC(Oc2ccc3c(ccn3-c3cc(Cl)c(OCCCl)c(C#N)c3)c2)CC1] |
| A203 | 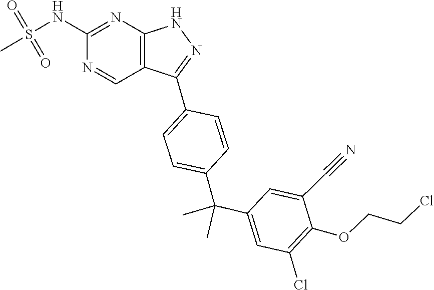 [SMILES: CC(C)(c1ccc(-c2n[nH]c3nc(NS(C)(=O)=O)ncc23)cc1)c1cc(Cl)c(OCCCl)c(C#N)c1] |
| A204 | 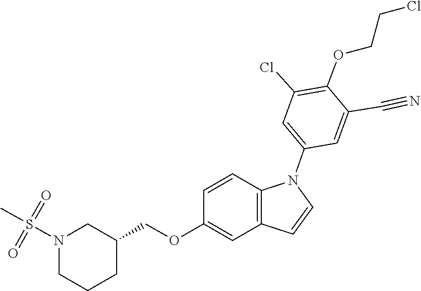 [SMILES: CS(=O)(=O)N1CCC[C@@H](COc2ccc3c(ccn3-c3cc(Cl)c(OCCCl)c(C#N)c3)c2)C1] |
| A205 | 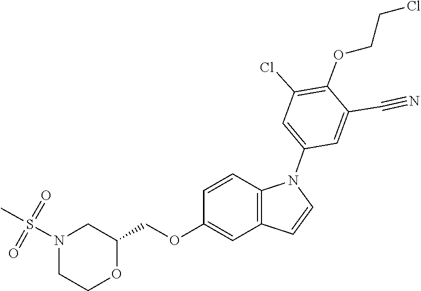 [SMILES: CS(=O)(=O)N1CCO[C@@H](COc2ccc3c(ccn3-c3cc(Cl)c(OCCCl)c(C#N)c3)c2)C1] |
| A206 | 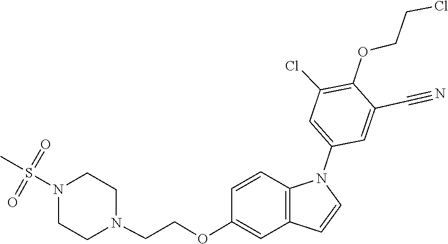 [SMILES: CS(=O)(=O)N1CCN(CCOc2ccc3c(ccn3-c3cc(Cl)c(OCCCl)c(C#N)c3)c2)CC1] |
| A207 | 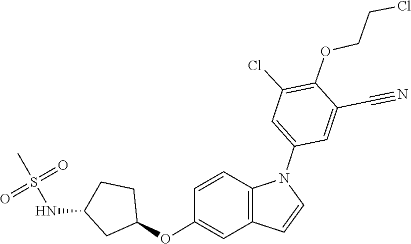 [SMILES: CS(=O)(=O)N[C@@H]1CC[C@@H](Oc2ccc3c(ccn3-c3cc(Cl)c(OCCCl)c(C#N)c3)c2)C1] |
| A208 | 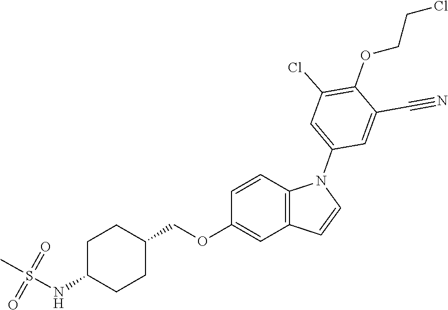 [SMILES: CS(=O)(=O)N[C@H]1CC[C@@H](COc2ccc3c(ccn3-c3cc(Cl)c(OCCCl)c(C#N)c3)c2)CC1] |
| A209 | 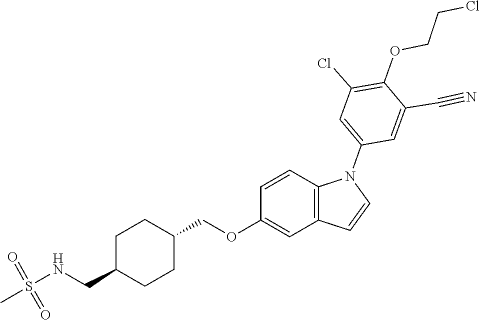 [SMILES: CS(=O)(=O)NC[C@H]1CC[C@H](COc2ccc3c(ccn3-c3cc(Cl)c(OCCCl)c(C#N)c3)c2)CC1] |
| A210 | 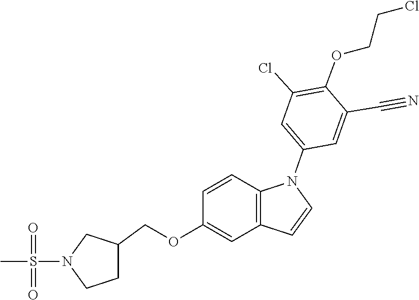 [SMILES: CS(=O)(=O)N1CCC(COc2ccc3c(ccn3-c3cc(Cl)c(OCCCl)c(C#N)c3)c2)C1] |
| A211 | 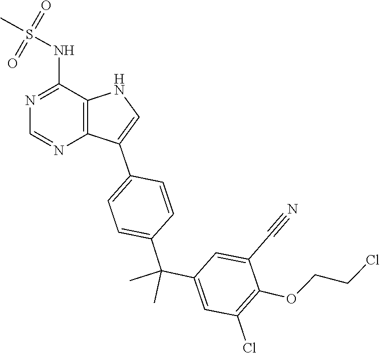 [SMILES: CC(C)(c1ccc(-c2c[nH]c3c(NS(C)(=O)=O)ncnc23)cc1)c1cc(Cl)c(OCCCl)c(C#N)c1] |
| A212 | 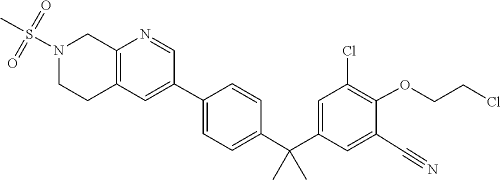 [SMILES: CC(C)(c1ccc(-c2cnc3c(c2)CCN(S(C)(=O)=O)C3)cc1)c1cc(Cl)c(OCCCl)c(C#N)c1] |
| A213 | 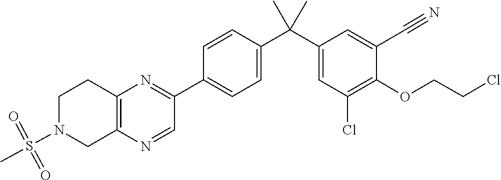 [SMILES: CC(C)(c1ccc(-c2cnc3c(n2)CCN(S(C)(=O)=O)C3)cc1)c1cc(Cl)c(OCCCl)c(C#N)c1] |
| A214 | 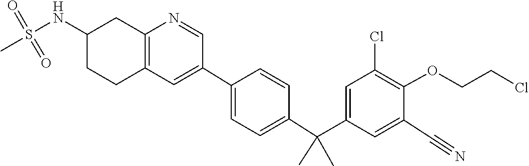 [SMILES: CC(C)(c1ccc(-c2cnc3c(c2)CCC(NS(C)(=O)=O)C3)cc1)c1cc(Cl)c(OCCCl)c(C#N)c1] |
| A215 | 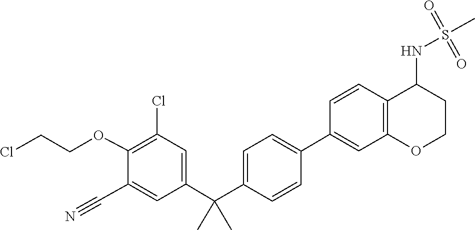 [SMILES: CC(C)(c1ccc(-c2ccc3c(c2)OCCC3NS(C)(=O)=O)cc1)c1cc(Cl)c(OCCCl)c(C#N)c1] |
| A216 | 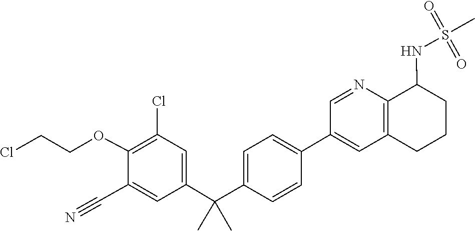 [SMILES: CC(C)(c1ccc(-c2cnc3c(c2)CCCC3NS(C)(=O)=O)cc1)c1cc(Cl)c(OCCCl)c(C#N)c1] |
| A217 | 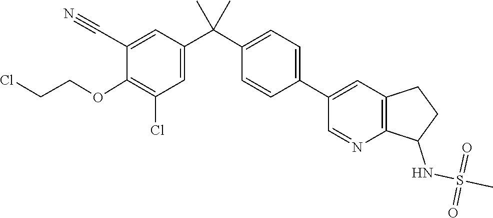 [SMILES: CC(C)(c1ccc(-c2cnc3c(c2)CCC3NS(C)(=O)=O)cc1)c1cc(Cl)c(OCCCl)c(C#N)c1] |
| A218 | 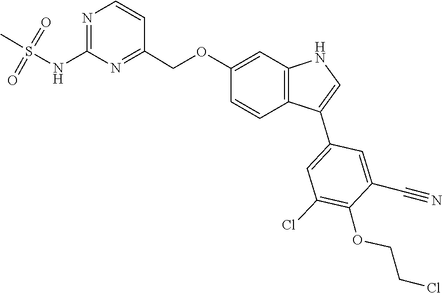 [SMILES: CS(=O)(=O)Nc1nccc(COc2ccc3c(-c4cc(Cl)c(OCCCl)c(C#N)c4)c[nH]c3c2)n1] |
| A219 | 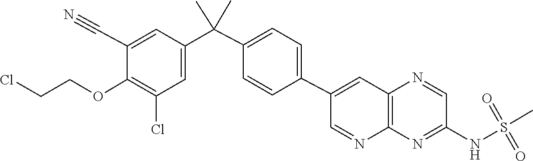 [SMILES: CC(C)(c1ccc(-c2cnc3nc(NS(C)(=O)=O)cnc3c2)cc1)c1cc(Cl)c(OCCCl)c(C#N)c1] |
| A220 | 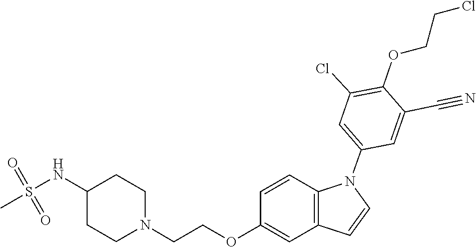 [SMILES: CS(=O)(=O)NC1CCN(CCOc2ccc3c(ccn3-c3cc(Cl)c(OCCCl)c(C#N)c3)c2)CC1] |
| A221 | 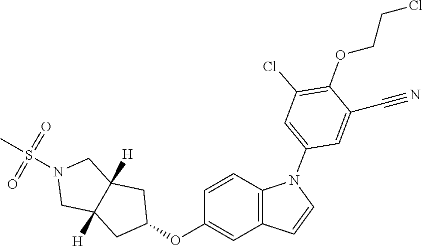 [SMILES: CS(=O)(=O)N1C[C@H]2C[C@@H](Oc3ccc4c(ccn4-c4cc(Cl)c(OCCCl)c(C#N)c4)c3)C[C@H]2C1] |
| A222 | 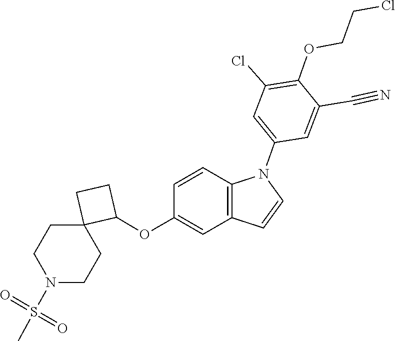 [SMILES: CS(=O)(=O)N1CCC2(CCC2Oc2ccc3c(ccn3-c3cc(Cl)c(OCCCl)c(C#N)c3)c2)CC1] |
| A223 | 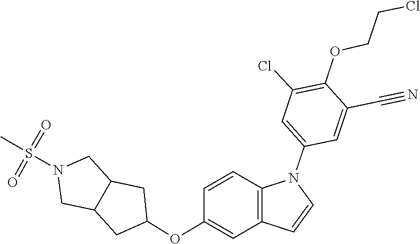 [SMILES: CS(=O)(=O)N1CC2CC(Oc3ccc4c(ccn4-c4cc(Cl)c(OCCCl)c(C#N)c4)c3)CC2C1] |
| A224 | 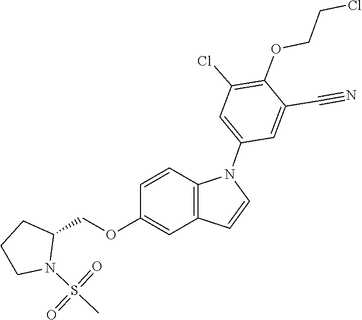 [SMILES: CS(=O)(=O)N1CCC[C@@H]1COc1ccc2c(ccn2-c2cc(Cl)c(OCCCl)c(C#N)c2)c1] |
| A225 | 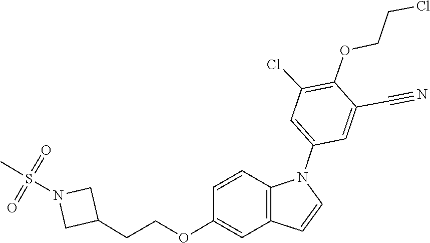 [SMILES: CS(=O)(=O)N1CC(CCOc2ccc3c(ccn3-c3cc(Cl)c(OCCCl)c(C#N)c3)c2)C1] |
| A226 | 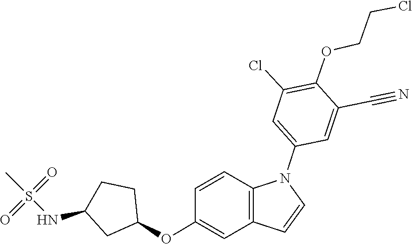 [SMILES: CS(=O)(=O)N[C@H]1CC[C@@H](Oc2ccc3c(ccn3-c3cc(Cl)c(OCCCl)c(C#N)c3)c2)C1] |
| A227 | 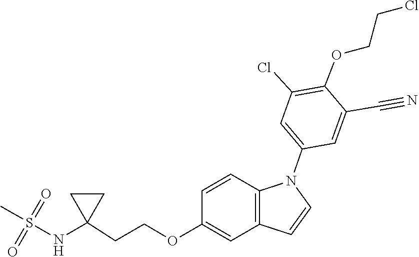 [SMILES: CS(=O)(=O)NC1(CCOc2ccc3c(ccn3-c3cc(Cl)c(OCCCl)c(C#N)c3)c2)CC1] |
| A228 | 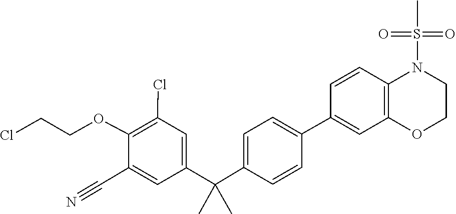 [SMILES: CC(C)(c1ccc(-c2ccc3c(c2)OCCN3S(C)(=O)=O)cc1)c1cc(Cl)c(OCCCl)c(C#N)c1] |
| A229 | 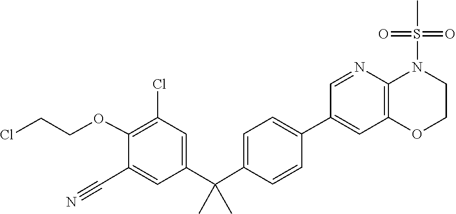 [SMILES: CC(C)(c1ccc(-c2cnc3c(c2)OCCN3S(C)(=O)=O)cc1)c1cc(Cl)c(OCCCl)c(C#N)c1] |
| A230 | 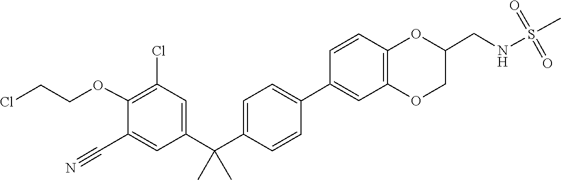 [SMILES: CC(C)(c1ccc(-c2ccc3c(c2)OCC(CNS(C)(=O)=O)O3)cc1)c1cc(Cl)c(OCCCl)c(C#N)c1] |
| A231 | 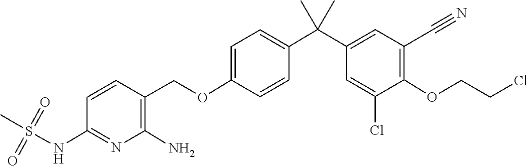 [SMILES: CC(C)(c1ccc(OCc2ccc(NS(C)(=O)=O)nc2N)cc1)c1cc(Cl)c(OCCCl)c(C#N)c1] |
| A232 | 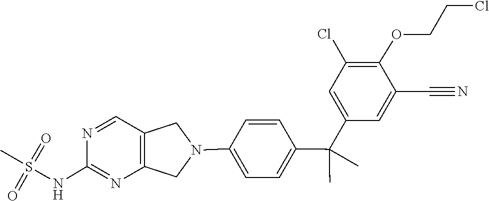 [SMILES: CC(C)(c1ccc(N2Cc3cnc(NS(C)(=O)=O)nc3C2)cc1)c1cc(Cl)c(OCCCl)c(C#N)c1] |
| A233 | 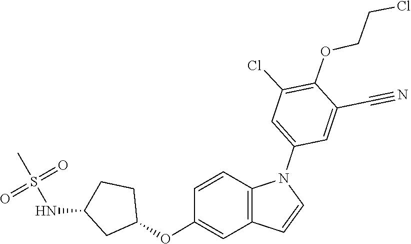 [SMILES: CS(=O)(=O)N[C@@H]1CC[C@H](Oc2ccc3c(ccn3-c3cc(Cl)c(OCCCl)c(C#N)c3)c2)C1] |
| A234 | 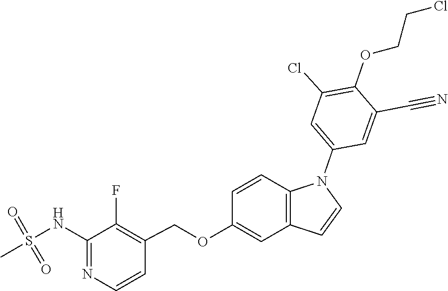 [SMILES: CS(=O)(=O)Nc1nccc(COc2ccc3c(ccn3-c3cc(Cl)c(OCCCl)c(C#N)c3)c2)c1F] |
| A235 | 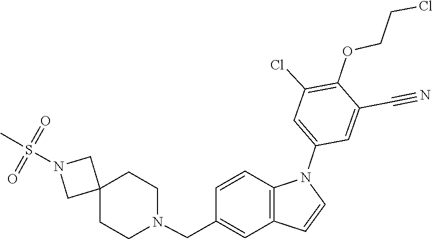 [SMILES: CS(=O)(=O)N1CC2(CCN(Cc3ccc4c(ccn4-c4cc(Cl)c(OCCCl)c(C#N)c4)c3)CC2)C1] |
| A236 | 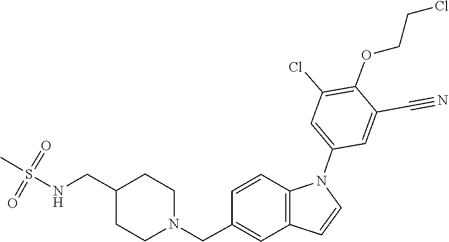 [SMILES: CS(=O)(=O)NCC1CCN(Cc2ccc3c(ccn3-c3cc(Cl)c(OCCCl)c(C#N)c3)c2)CC1] |
| A237 | 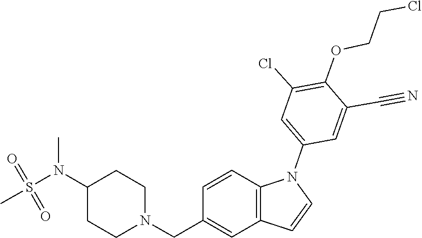 [SMILES: CN(C1CCN(Cc2ccc3c(ccn3-c3cc(Cl)c(OCCCl)c(C#N)c3)c2)CC1)S(C)(=O)=O] |
| A238 | 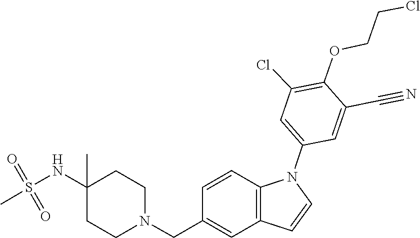 [SMILES: CC1(NS(C)(=O)=O)CCN(Cc2ccc3c(ccn3-c3cc(Cl)c(OCCCl)c(C#N)c3)c2)CC1] |
| A239 | 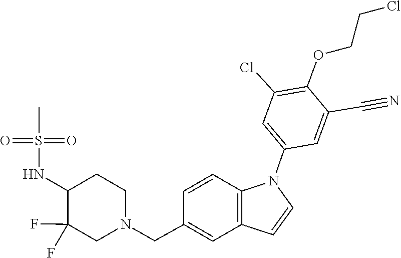 [SMILES: CS(=O)(=O)NC1CCN(Cc2ccc3c(ccn3-c3cc(Cl)c(OCCCl)c(C#N)c3)c2)CC1(F)F] |
| A240 | 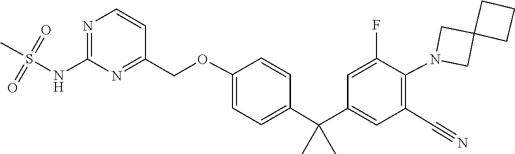 [SMILES: CC(C)(c1ccc(OCc2ccnc(NS(C)(=O)=O)n2)cc1)c1cc(F)c(N2CC3(CCC3)C2)c(C#N)c1] |
| A241 | 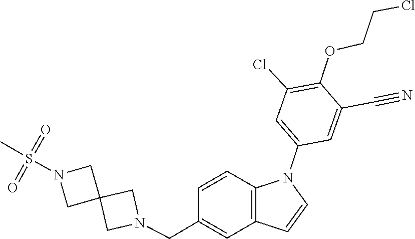 [SMILES: CS(=O)(=O)N1CC2(CN(Cc3ccc4c(ccn4-c4cc(Cl)c(OCCCl)c(C#N)c4)c3)C2)C1] |
| A242 | 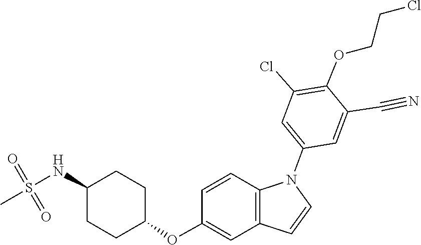 [SMILES: CS(=O)(=O)N[C@H]1CC[C@H](Oc2ccc3c(ccn3-c3cc(Cl)c(OCCCl)c(C#N)c3)c2)CC1] |
| A243 | 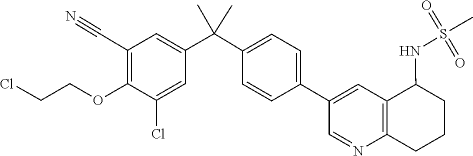 [SMILES: CC(C)(c1ccc(-c2cnc3c(c2)C(NS(C)(=O)=O)CCC3)cc1)c1cc(Cl)c(OCCCl)c(C#N)c1] |
| A244 | 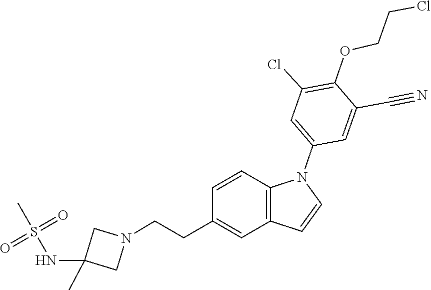 [SMILES: CC1(NS(C)(=O)=O)CN(CCc2ccc3c(ccn3-c3cc(Cl)c(OCCCl)c(C#N)c3)c2)C1] |
| A245 | 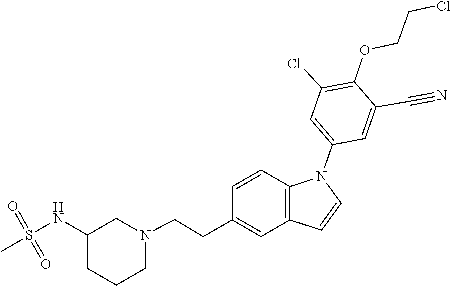 [SMILES: CS(=O)(=O)NC1CCCN(CCc2ccc3c(ccn3-c3cc(Cl)c(OCCCl)c(C#N)c3)c2)C1] |
| A246 | 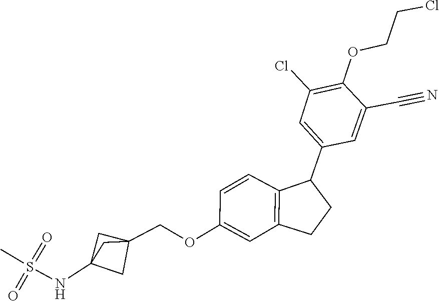 [SMILES: CS(=O)(=O)NC12CC(COc3ccc4c(c3)CCC4c3cc(Cl)c(OCCCl)c(C#N)c3)(C1)C2] |
| A247 | 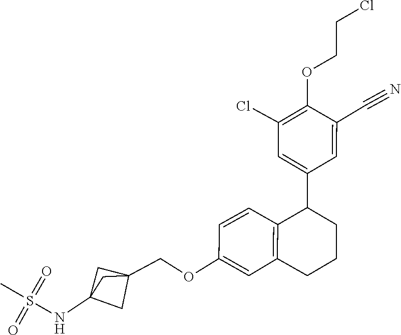 [SMILES: CS(=O)(=O)NC12CC(COc3ccc4c(c3)CCCC4c3cc(Cl)c(OCCCl)c(C#N)c3)(C1)C2] |
| A248 | 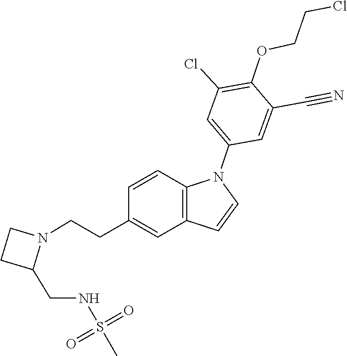 [SMILES: CS(=O)(=O)NCC1CCN1CCc1ccc2c(ccn2-c2cc(Cl)c(OCCCl)c(C#N)c2)c1] |
| A249 | 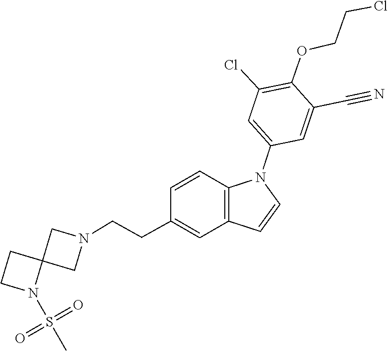 [SMILES: CS(=O)(=O)N1CCC12CN(CCc1ccc3c(ccn3-c3cc(Cl)c(OCCCl)c(C#N)c3)c1)C2] |
| A250 | 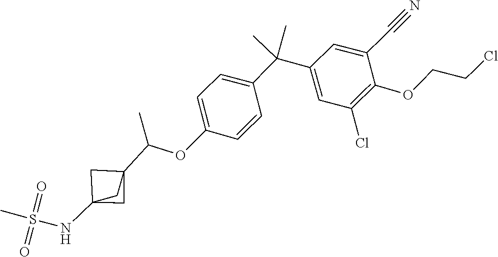 [SMILES: CC(Oc1ccc(C(C)(C)c2cc(Cl)c(OCCCl)c(C#N)c2)cc1)C12CC(NS(C)(=O)=O)(C1)C2] |
| A251 | 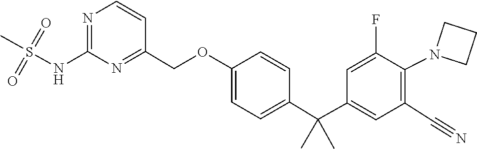 [SMILES: CC(C)(c1ccc(OCc2ccnc(NS(C)(=O)=O)n2)cc1)c1cc(F)c(N2CCC2)c(C#N)c1] |
| A252 | 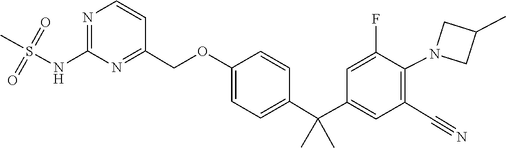 |
| A253 | 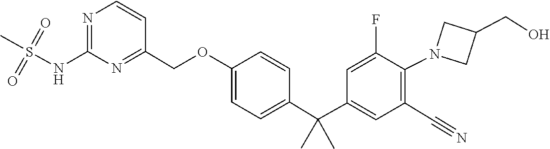 [SMILES: CC(C)(c1ccc(OCc2ccnc(NS(C)(=O)=O)n2)cc1)c1cc(F)c(N2CC(CO)C2)c(C#N)c1] |
| A254 | 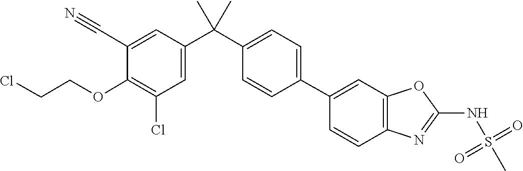 [SMILES: CC(C)(c1ccc(-c2ccc3nc(NS(C)(=O)=O)oc3c2)cc1)c1cc(Cl)c(OCCCl)c(C#N)c1] |
| A255 | 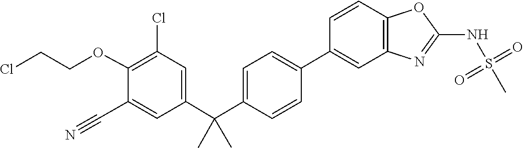 [SMILES: CC(C)(c1ccc(-c2ccc3oc(NS(C)(=O)=O)nc3c2)cc1)c1cc(Cl)c(OCCCl)c(C#N)c1] |
| A256 | 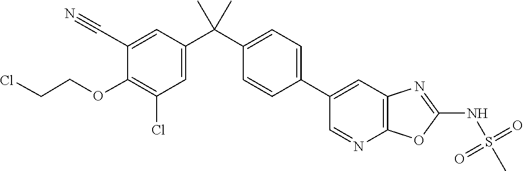 [SMILES: CC(C)(c1ccc(-c2cnc3oc(NS(C)(=O)=O)nc3c2)cc1)c1cc(Cl)c(OCCCl)c(C#N)c1] |
| A257 | 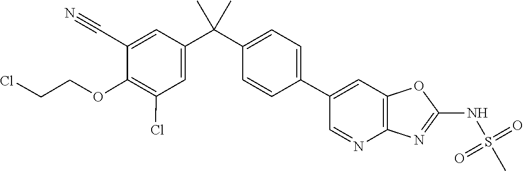 [SMILES: CC(C)(c1ccc(-c2cnc3nc(NS(C)(=O)=O)oc3c2)cc1)c1cc(Cl)c(OCCCl)c(C#N)c1] |
| A258 | 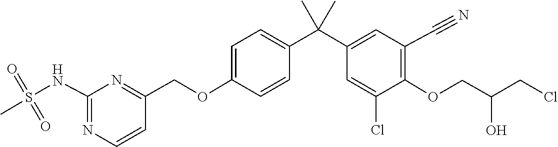 [SMILES: CC(C)(c1ccc(OCc2ccnc(NS(C)(=O)=O)n2)cc1)c1cc(Cl)c(OCC(O)CCl)c(C#N)c1] |
| A259 | 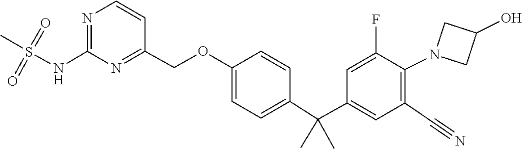 [SMILES: CC(C)(c1ccc(OCc2ccnc(NS(C)(=O)=O)n2)cc1)c1cc(F)c(N2CC(O)C2)c(C#N)c1] |
| A260 | 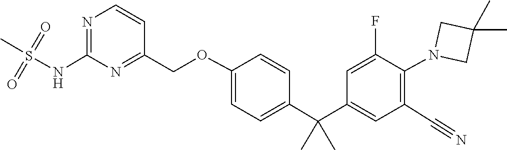 |
| A261 | 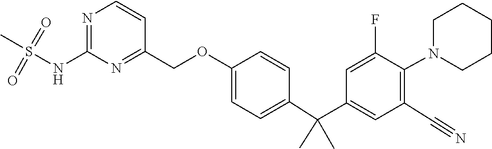 [SMILES: CC(C)(c1ccc(OCc2ccnc(NS(C)(=O)=O)n2)cc1)c1cc(F)c(N2CCCCC2)c(C#N)c1] |
| A262 | 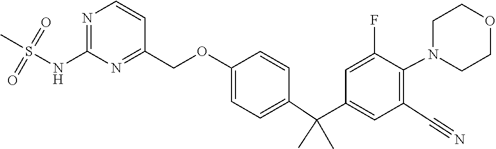 [SMILES: CC(C)(c1ccc(OCc2ccnc(NS(C)(=O)=O)n2)cc1)c1cc(F)c(N2CCOCC2)c(C#N)c1] |
| A263 | 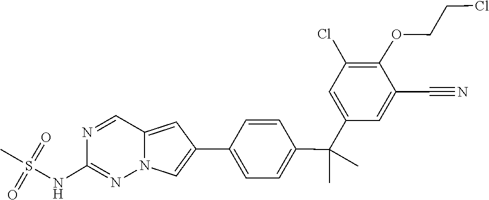 [SMILES: CC(C)(c1ccc(-c2cc3cnc(NS(C)(=O)=O)nn3c2)cc1)c1cc(Cl)c(OCCCl)c(C#N)c1] |
| A264 | 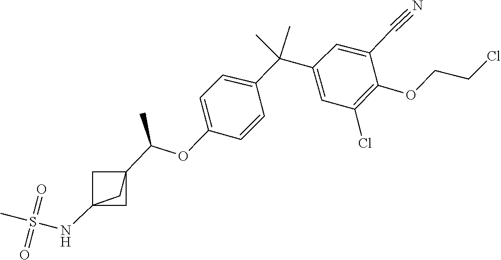 [SMILES: C[C@@H](Oc1ccc(C(C)(C)c2cc(Cl)c(OCCCl)c(C#N)c2)cc1)C12CC(NS(C)(=O)=O)(C1)C2] |
| A265 | 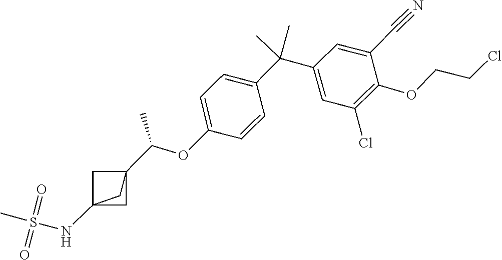 [SMILES: C[C@H](Oc1ccc(C(C)(C)c2cc(Cl)c(OCCCl)c(C#N)c2)cc1)C12CC(NS(C)(=O)=O)(C1)C2] |
| A266 | 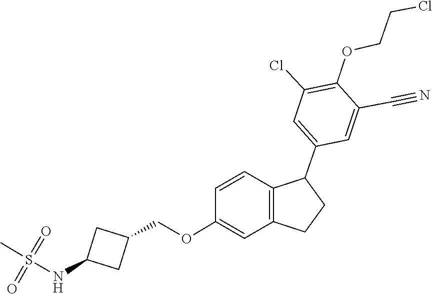 [SMILES: CS(=O)(=O)N[C@H]1C[C@H](COc2ccc3c(c2)CCC3c2cc(Cl)c(OCCCl)c(C#N)c2)C1] |
| A267 | 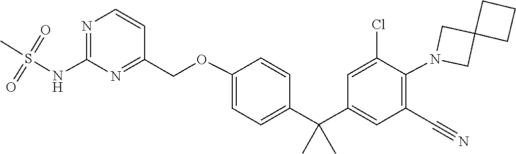 [SMILES: CC(C)(c1ccc(OCc2ccnc(NS(C)(=O)=O)n2)cc1)c1cc(Cl)c(N2CC3(CCC3)C2)c(C#N)c1] |
| A268 | 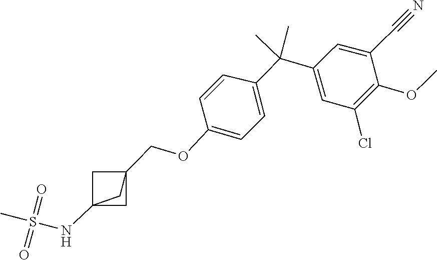 |
| A269 | 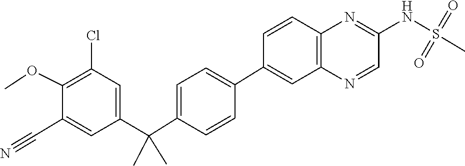 |
| A270 | 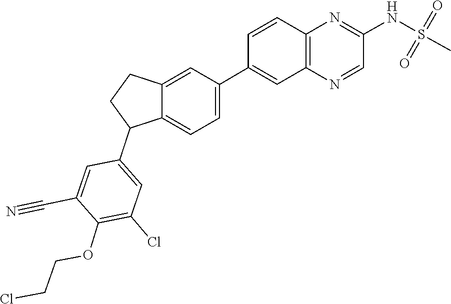 [SMILES: CS(=O)(=O)Nc1cnc2cc(-c3ccc4c(c3)CCC4c3cc(Cl)c(OCCCl)c(C#N)c3)ccc2n1] |
| A271 | 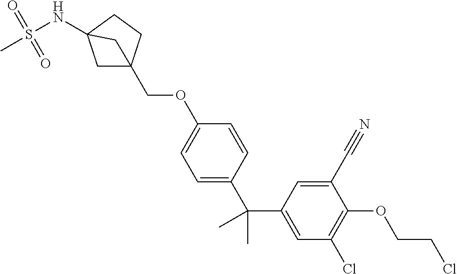 [SMILES: CC(C)(c1ccc(OCC23CCC(NS(C)(=O)=O)(C2)C3)cc1)c1cc(Cl)c(OCCCl)c(C#N)c1] |
| A272 | 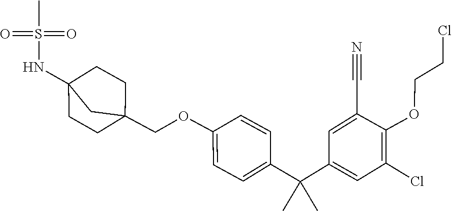 [SMILES: CC(C)(c1ccc(OCC23CCC(NS(C)(=O)=O)(CC2)C3)cc1)c1cc(Cl)c(OCCCl)c(C#N)c1] |
| A273 | 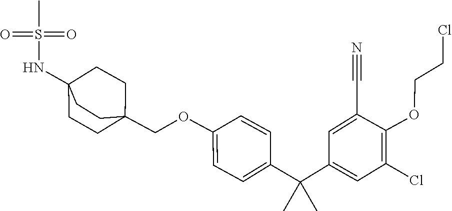 [SMILES: CC(C)(c1ccc(OCC23CCC(NS(C)(=O)=O)(CC2)CC3)cc1)c1cc(Cl)c(OCCCl)c(C#N)c1] |
| A274 | 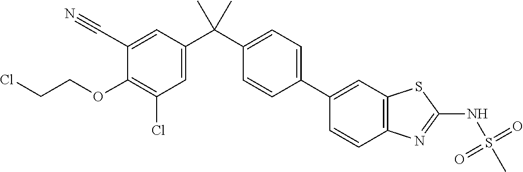 [SMILES: CC(C)(c1ccc(-c2ccc3nc(NS(C)(=O)=O)sc3c2)cc1)c1cc(Cl)c(OCCCl)c(C#N)c1] |
| A275 | 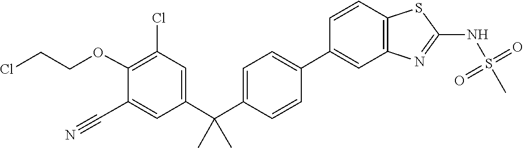 [SMILES: CC(C)(c1ccc(-c2ccc3sc(NS(C)(=O)=O)nc3c2)cc1)c1cc(Cl)c(OCCCl)c(C#N)c1] |
| A276 | 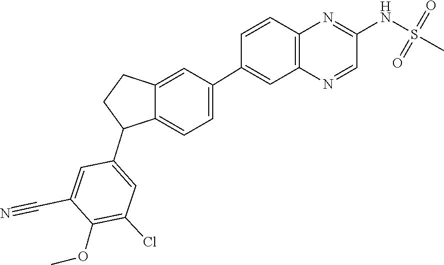 [SMILES: COc1c(Cl)cc(C2CCc3cc(-c4ccc5nc(NS(C)(=O)=O)cnc5c4)ccc32)cc1C#N] |
| A277 | 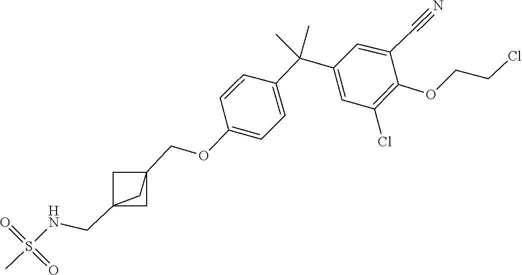 [SMILES: CC(C)(c1ccc(OCC23CC(CNS(C)(=O)=O)(C2)C3)cc1)c1cc(Cl)c(OCCCl)c(C#N)c1] |
| A278 | 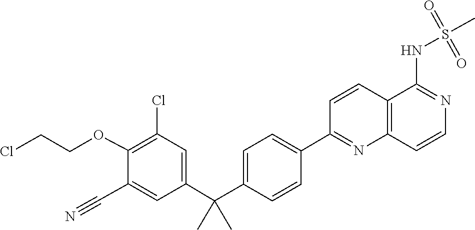 [SMILES: CC(C)(c1ccc(-c2ccc3c(NS(C)(=O)=O)nccc3n2)cc1)c1cc(Cl)c(OCCCl)c(C#N)c1] |
| A279 | 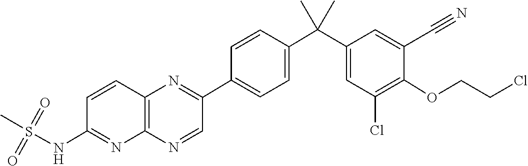 [SMILES: CC(C)(c1ccc(-c2cnc3nc(NS(C)(=O)=O)ccc3n2)cc1)c1cc(Cl)c(OCCCl)c(C#N)c1] |
| A280 | 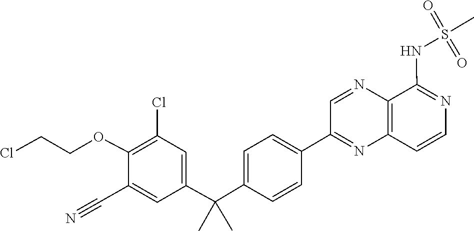 [SMILES: CC(C)(c1ccc(-c2cnc3c(NS(C)(=O)=O)nccc3n2)cc1)c1cc(Cl)c(OCCCl)c(C#N)c1] |
| A281 | 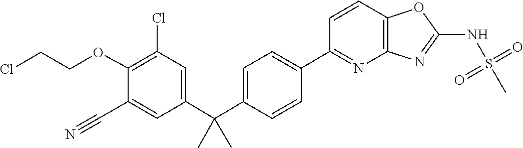 [SMILES: CC(C)(c1ccc(-c2ccc3oc(NS(C)(=O)=O)nc3n2)cc1)c1cc(Cl)c(OCCCl)c(C#N)c1] |
| A282 | 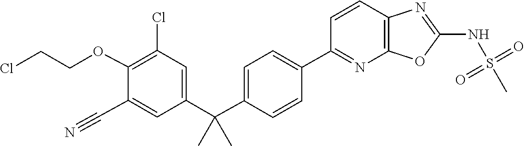 [SMILES: CC(C)(c1ccc(-c2ccc3nc(NS(C)(=O)=O)oc3n2)cc1)c1cc(Cl)c(OCCCl)c(C#N)c1] |
| A283 | 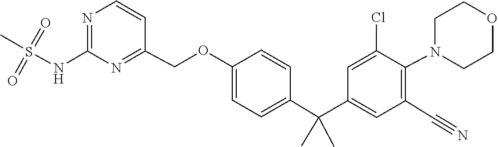 [SMILES: CC(C)(c1ccc(OCc2ccnc(NS(C)(=O)=O)n2)cc1)c1cc(Cl)c(N2CCOCC2)c(C#N)c1] |
| A284 | 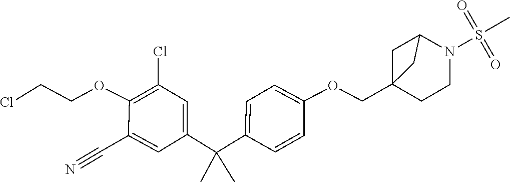 [SMILES: CC(C)(c1ccc(OCC23CCN(S(C)(=O)=O)C(C2)C3)cc1)c1cc(Cl)c(OCCCl)c(C#N)c1] |
| A285 | 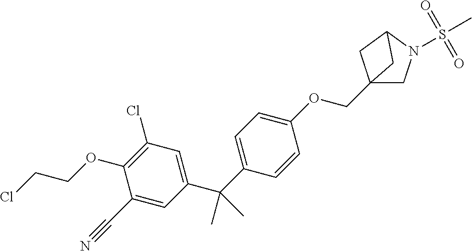 [SMILES: CC(C)(c1ccc(OCC23CC(C2)N(S(C)(=O)=O)C3)cc1)c1cc(Cl)c(OCCCl)c(C#N)c1] |

In one embodiment, the present disclosure provides a pharmaceutical composition comprising a pharmaceutically acceptable carrier or a pharmaceutically acceptable excipient and a compound of any one of formula (I), (I-A), (I-B), (II), (A), (A-I), (A-II), (A-III), (B), (B-I), (C), (D), (E), (E-I), (F), (G), (H), (J), and/or (K), or compounds of Table A, or a pharmaceutically acceptable salt, tautomer, stereoisomer or prodrug thereof. In one embodiment, the present disclosure provides a pharmaceutical composition comprising a pharmaceutically acceptable carrier or a pharmaceutically acceptable excipient and a compound of any one of formula (II), (A), (A-I), (A-II), (A-III), (B), (B-I), (C), (D), (E), (E-I), (F), (G), (H), (J), and/or (K), or compounds of Table A, or a pharmaceutically acceptable salt, tautomer, stereoisomer or prodrug thereof. In one embodiment, the present disclosure provides a pharmaceutical composition comprising a pharmaceutically acceptable carrier or a pharmaceutically acceptable excipient and a compound of any one of formula (A), (A-I), (A-II), (A-III), (B), (B-I), (C), (D), (E), (E-I), (F), (G), (H), (J), and/or (K), or compounds of Table A, or a pharmaceutically acceptable salt, tautomer, stereoisomer or prodrug thereof.

a, or a pharmaceutically acceptable salt, tautomer, stereoisomer or prodrug thereof.

In one embodiment, the present disclosure provides a pharmaceutical composition comprising a pharmaceutically acceptable carrier or a pharmaceutically acceptable excipient and a compound selected from Compounds A1-A41 (including A35(R) and A35(S)) or a pharmaceutically acceptable salt, tautomer, stereoisomer or prodrug thereof. In one embodiment, the present disclosure provides a pharmaceutical composition comprising a pharmaceutically acceptable carrier or a pharmaceutically acceptable excipient and a compound selected from Compounds A1-A199 (including A35(R) and A35(S)) or a pharmaceutically acceptable salt, tautomer, stereoisomer or prodrug thereof. In one embodiment, the present disclosure provides a pharmaceutical composition comprising a pharmaceutically acceptable carrier or a pharmaceutically acceptable excipient and a compound selected from Compounds A1-A285 (including A35(R) and A35(S)) or a pharmaceutically acceptable salt, tautomer, stereoisomer or prodrug thereof.

### Therapeutic Use

The present compounds find use in any number of methods. For example, in some embodiments the compounds are useful in methods for modulating androgen receptor (AR). Accordingly, in one embodiment, the present disclosure provides the use of any one of the foregoing compounds of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer, stereoisomer or prodrug thereof, for modulating androgen receptor (AR) activity. For example, in some embodiments, modulating androgen receptor (AR) activity is in a mammalian cell. Modulating androgen receptor (AR) can be in a subject in need thereof (e.g., a mammalian subject) and for treatment of any of the described conditions or diseases.

In one embodiment, the modulating AR is binding to AR. In other embodiments, the modulating AR is inhibiting AR.

In one embodiment, the modulating AR is modulating AR N-terminal domain (NTD). In one embodiment, the modulating AR is binding to AR NTD. In other embodiments, the modulating AR is inhibiting AR NTD. In one embodiment, the modulating AR is modulating AR N-terminal domain (NTD). In some embodiments, modulating the AR is inhibiting transactivation of androgen receptor N-terminal domain (NTD).

In other embodiments, modulating androgen receptor (AR) activity is for treatment of at least one indication selected from the group consisting of: prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, age related macular degeneration, and combinations thereof. For example, in some embodiments, the indication is prostate cancer. In other embodiments, the prostate cancer is primary/localized prostate cancer, locally advanced prostate cancer, recurrent prostate cancer, non-metastatic castration-resistant prostate cancer, metastatic prostate cancer, advanced prostate cancer, or metastatic castration-resistant prostate cancer (CRPC), or hormone-sensitive prostate cancer. While in other embodiments, the prostate cancer is androgen dependent prostate cancer. In other embodiments, the spinal and bulbar muscular atrophy is Kennedy's disease. In other embodiments, the prostate cancer is primary/localized prostate cancer, locally advanced prostate cancer, recurrent prostate cancer, metastatic prostate cancer, advanced prostate cancer, or metastatic castration-resistant prostate cancer (CRPC), or hormone-sensitive prostate cancer. While in other embodiments, the prostate cancer is androgen dependent prostate cancer. In other embodiments, the spinal and bulbar muscular atrophy is Kennedy's disease.

In one embodiment of the present disclosure, a method of treating a condition associated with cell proliferation in a patient in need thereof is provided, comprising administering a compounds of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer, stereoisomer or prodrug thereof, to a subject in need thereof. In one embodiment, the present invention provides a method of treating cancer or tumors. In another embodiment, the present invention provides a method of treating prostate cancer or breast cancer.

In one embodiment of the present disclosure, a method of reducing, inhibiting, or ameliorating proliferation, comprising administering a therapeutically effective amount of a compound of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof is provided. In one embodiment, the reducing, inhibiting, or ameliorating in the method disclosed herein, is in vivo. In another embodiment, the reducing, inhibiting, or ameliorating is in vitro.

In one embodiment, the cells in the method disclosed herein, are a cancer cells. In one embodiment, the cancer cells are a prostate cancer cells. In one embodiment, the prostate cancer cells are cells of primary/localized prostate cancer (newly diagnosed or early stage), locally advanced prostate cancer, recurrent prostate cancer (e.g., prostate cancer which was not responsive to primary therapy), metastatic prostate cancer, non-metastatic castration-resistant prostate cancer, advanced prostate cancer (e.g., after castration for recurrent prostate cancer), metastatic castration-resistant prostate cancer (CRPC), or hormone-sensitive prostate cancer. In one embodiment, the prostate cancer cells are cells of primary/localized prostate cancer (newly diagnosed or early stage), locally advanced prostate cancer, recurrent prostate cancer (e.g., prostate cancer which was not responsive to primary therapy), metastatic prostate cancer, advanced prostate cancer (e.g., after castration for recurrent prostate cancer), metastatic castration-resistant prostate cancer (CRPC), or hormone-sensitive prostate cancer. In another embodiment, the prostate cancer cells are cells of a metastatic castration-resistant prostate cancer. In other embodiments, the prostate cancer cells are an androgen-dependent prostate cancer cells or an androgen-independent prostate cancer cells. In one embodiment, the cancer cells are breast cancer cells.

In one embodiment, the condition or disease associated with cell proliferation is cancer. In one embodiment of any one of the methods disclosed herein, the cancer is selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration. In one embodiment, the condition or disease is prostate cancer. In one embodiment, prostate cancer is selected from primary/localized prostate cancer, locally advanced prostate cancer, recurrent prostate cancer, metastatic prostate cancer, non-metastatic castration-resistant prostate cancer, advanced prostate cancer, metastatic castration-resistant prostate cancer (CRPC), or hormone-sensitive prostate cancer. In one embodiment, prostate cancer is selected from primary/localized prostate cancer, locally advanced prostate cancer, recurrent prostate cancer, metastatic prostate cancer, advanced prostate cancer, metastatic castration-resistant prostate cancer (CRPC), or hormone-sensitive prostate cancer. In another embodiment, the prostate cancer is a metastatic castration-resistant prostate cancer. In some embodiments, the prostate cancer is an androgen-dependent prostate cancer cells or an androgen-independent prostate cancer. In one embodiment, the condition or disease is breast cancer.

In another embodiment of the present disclosure, a method for reducing or preventing tumor growth, comprising contacting tumor cells with a therapeutically effective amount of a compound of (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof is provided.

In one embodiment, reducing or preventing tumor growth includes reduction in tumor volume. In one embodiment, reducing or preventing tumor growth includes complete elimination of tumors. In one embodiment, reducing or preventing tumor growth includes stopping or halting the existing tumor to grow. In one embodiment, reducing or preventing tumor growth includes reduction in the rate of tumor growth. In one embodiment, reducing or preventing tumor growth includes reduction in the rate of tumor growth such that the rate of tumor growth before treating a patient with the methods disclosed herein (r1) is faster than the rate of tumor growth after said treatment (r2) such that r1>r2.

In one embodiment, the reducing or preventing in the method disclosed herein is in vivo. In another embodiment, the treating is in vitro.

In one embodiment, the tumor cell in the method disclosed herein is selected from prostate cancer, breast cancer, ovarian cancer, endometrial cancer, or salivary gland carcinoma. In one embodiment, the tumor cells are prostate cancer tumor cells. In one embodiment, the prostate cancer tumor cells are tumor cells of primary/localized prostate cancer, locally advanced prostate cancer, recurrent prostate cancer, metastatic prostate cancer, non-metastatic castration-resistant prostate cancer, advanced prostate cancer, metastatic castration-resistant prostate cancer (CRPC), or hormone-sensitive prostate cancer. In one embodiment, the prostate cancer tumor cells are tumor cells of primary/localized prostate cancer, locally advanced prostate cancer, recurrent prostate cancer, metastatic prostate cancer, advanced prostate cancer, metastatic castration-resistant prostate cancer (CRPC), or hormone-sensitive prostate cancer. In other embodiments, the prostate cancer is a metastatic castration-resistant prostate cancer. In some embodiments, the prostate cancer is androgen-dependent prostate cancer or androgen-independent prostate cancer. In another embodiment, the tumor cells are is breast cancer tumor cells.

In one embodiment, the present disclosure provides compounds which demonstrate blocking androgen-induced PSA-luciferase activity (PSA-luciferase assay). In a specific embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro IC₅₀ in a PSA-luciferase assay of less than about 4500 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro IC₅₀ in a PSA-luciferase assay of less than about 4000 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro IC₅₀ in a PSA-luciferase assay of less than about 3500 nM. In one embodiment, the compounds of the present disclosure are compounds having the structure of formula (I), (I-A), (I-B) or, (II), (A), (A-I), (A-II), (A-III) (B), (B-I), (C), (D), (E), (E-I), (F) (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro IC₅₀ in a PSA-luciferase assay of less than about 3000 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro IC₅₀ in a PSA-luciferase assay of less than about 2500 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro IC₅₀ in a PSA-luciferase assay of less than about 2000 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro IC₅₀ in a PSA-luciferase assay of less than about 1500 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro IC₅₀ in a PSA-luciferase assay of less than about 1000 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro IC₅₀ in a PSA-luciferase assay of less than about 950 nm. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro IC₅₀ in a PSA-luciferase assay of less than about 900 nM.

In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro IC₅₀ in a PSA-luciferase assay of less than about 890 nM, less than about 880 nM, less than about 870 nM, less than about 860 nM, less than about 850 nM, less than about 840 nM, less than about 830 nM, less than about 820 nM, less than about 810 nM, or less than about 800 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro IC₅₀ in a PSA-luciferase assay of less than about 790 nM, less than about 780 nM, less than about 770 nM, less than about 760 nM, less than about 750 nM, less than about 740 nM, less than about 730 nM, less than about 720 nM, less than about 710 nM, or less than about 700 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro IC₅₀ in a PSA-luciferase assay of less than about 690 nM, less than about 680 nM, less than about 670 nM, less than about 660 nM, less than about 650 nM, less than about 640 nM, less than about 630 nM, less than about 620 nM, less than about 610 nM, or less than about 600 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro IC₅₀ in a PSA-luciferase assay of less than about 590 nM, less than about 580 nM, less than about 570 nM, less than about 560 nM, less than about 550 nM, less than about 540 nM, less than about 530 nM, less than about 520 nM, less than about 510 nM, or less than about 500 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro IC₅₀ in a PSA-luciferase assay of less than about 500 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds having in vitro IC₅₀ in a PSA-luciferase assay of less than about 490 nM, less than about 480 nM, less than about 470 nM, less than about 460 nM, less than about 450 nM, less than about 440 nM, less than about 430 nM, less than about 420 nM, less than about 410 nM, or less than about 400 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro IC₅₀ in a PSA-luciferase assay of less than about 390 nM, less than about 380 nM, less than about 370 nM, less than about 360 nM, less than about 350 nM, less than about 340 nM, less than about 330 nM, less than about 320 nM, less than about 310 nM, or less than about 300 nM. In one embodiment, the compounds of the present disclosure are compounds having the structure of formula (I), (I-A), (I-B), (II), (A), (A-I), (A-II), (A-III), (B) C)tautomer, (D), (E), (E-I), (F), He, (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro IC₅₀ in a PSA-luciferase assay of less than about 290 nM, less than about 280 nM, less than about 270 nM, less than about 260 nM, less than about 250 nM, less than about 240 nM, less than about 230 nM, less than about 220 nM, less than about 210 nM, or less than about 200 nM. In one embodiment, the compounds have an in vitro IC₅₀ in a PSA-luciferase assay of less than about 200 nM. In one embodiment, the compound is selected from any of the compound of Table A or a pharmaceutically acceptable salt, tautomer or stereoisomer thereof.

In another embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro half-life in mammal microsome of greater than about 80 minutes and in vitro IC₅₀ in a PSA-luciferase assay of less than about 2000 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro half-life in mammal microsome of greater than about 90 minutes and in vitro IC₅₀ in a PSA-luciferase assay of less than about 1500 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro half-life in mammal microsome of greater than about 100 minutes and in vitro IC₅₀ in a PSA-luciferase assay of less than about 1000 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro half-life in mammal microsome of greater than about 105 minutes and in vitro IC₅₀ in a PSA-luciferase assay of less than about 900 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro half-life in mammal microsome of greater than about 110 minutes and in vitro IC₅₀ in a PSA-luciferase assay of less than about 850 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro half-life in mammal microsome of greater than about 115 minutes and in vitro IC₅₀ in a PSA-luciferase assay of less than about 800 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro half-life in mammal microsome of greater than about 115 minutes and in vitro IC₅₀ in a PSA-luciferase assay of less than about 750 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro half-life in mammal microsome of greater than about 120 minutes and in vitro IC₅₀ in a PSA-luciferase assay of less than about 700 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro half-life in mammal microsome of greater than about 120 minutes and in vitro IC₅₀ in a PSA-luciferase assay of less than about 650 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro half-life in mammal microsome of greater than about 120 minutes and in vitro IC₅₀ in a PSA-luciferase assay of less than about 600 nM. In one embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, or the compounds of Table A, or a pharmaceutically acceptable salt, tautomer or stereoisomer thereof, wherein the compounds have an in vitro half-life in mammal microsome of greater than about 120 minutes and in vitro IC₅₀ in a PSA-luciferase assay of less than about 550 nM.

In a specific embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, and wherein the compounds have an in vitro IC₅₀ in a PSA-luciferase assay of less than 650 nM. In one embodiment, the compounds have an in vitro IC₅₀ in a PSA-luciferase assay of less than about 640 nM. In one embodiment, the compounds have an in vitro IC₅₀ in a PSA-luciferase assay of less than about 635 nM.

In a specific embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, and wherein the compounds have an in vitro IC₅₀ in a PSA-luciferase assay in the range of 200 nM to about 700 nM. In one embodiment, the compounds have an in vitro IC₅₀ in a PSA-luciferase assay in the range of about 250 nM to about 700 nM. In one embodiment, the compounds have an in vitro IC₅₀ in a PSA-luciferase assay in the range of about 300 nM to about 700 nM. In one embodiment, the compounds have an in vitro IC₅₀ in a PSA-luciferase assay in the range of about 350 nM to about 675 nM.

In a specific embodiment, the compounds of the present disclosure are compounds having the structure of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt, tautomer or stereoisomer thereof, and wherein the compounds have an in vitro IC₅₀ in a PSA-luciferase assay of less than 525 nM. In one embodiment, the compounds have an in vitro IC₅₀ in a PSA-luciferase assay of less than about 500 nM. In one embodiment, the compounds have an in vitro IC₅₀ in a PSA-luciferase assay of less than about 450 nM.

### Pharmaceutical Compositions and Formulations

The present disclosure also includes pharmaceutical compositions for modulating androgen receptor (AR) in a subject. In one embodiment, a pharmaceutical composition comprises one or more compounds of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt or solvate thereof.

In one embodiment of the present disclosure, a pharmaceutical composition comprises a therapeutically effective amounts of one or more compounds of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt or solvate thereof.

In a specific embodiment, a pharmaceutical composition, as described herein, comprises one or more compounds selected from Table A, or a pharmaceutically acceptable salt or solvate thereof.

In a specific embodiment, a pharmaceutical composition, as described herein, comprises one or more compounds selected from Compounds A1-A285 (including A35(R) and A35(S)) or a pharmaceutically acceptable salt or solvate thereof.

In one embodiment, a pharmaceutical composition, as described herein, comprising one or more compounds of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt or solvate thereof, further comprises one or more additional therapeutically active agents. In one embodiment, one or more additional therapeutically active agents are selected from therapeutics useful for treating cancer, neurological disease, a disorder characterized by abnormal accumulation of α-synuclein, a disorder of an aging process, cardiovascular disease, bacterial infection, viral infection, mitochondrial related disease, mental retardation, deafness, blindness, diabetes, obesity, autoimmune disease, glaucoma, Leber's Hereditary Optic Neuropathy, and rheumatoid arthritis.

In some embodiments, the one or more additional therapeutic agents is a poly (ADP-ribose) polymerase (PARP) inhibitor including but not limited to olaparib, niraparib, rucaparib, talazoparib; an androgen receptor ligand binding domain inhibitor including but not limited to enzalutamide, apalutamide, darolutamide, bicalutamide, nilutamide, flutamide, ODM-204, TAS3681; an inhibitor of CYP17 including but not limited to galeterone, abiraterone, abiraterone acetate; a microtubule inhibitor including but not limited to docetaxel, paclitaxel, cabazitaxel (XRP-6258); a modulator of PD-1 or PD-L1 including but not limited to pembrolizumab, durvalumab, nivolumab, atezolizumab; a gonadotropin releasing hormone agonist including but not limited to cyproterone acetate, leuprolide, a 5-alpha reductase inhibitor including but not limited to finasteride, dutasteride, turosteride, bexlosteride, izonsteride, FCE 28260, SKF105,111; a vascular endothelial growth factor inhibitor including but not limited to bevacizumab (Avastin); a histone deacetylase inhibitor including but not limited to OSU-HDAC42; an integrin alpha-v-beta-3 inhibitor including but not limited to VITAXIN; a receptor tyrosine kinase inhibitor including but not limited to sunitumib; a phosphoinositide 3-kinase inhibitor including but not limited to alpelisib, buparlisib, idealisib; an anaplastic lymphoma kinase (ALK) inhibitor including but not limited to crizotinib, alectinib; an endothelin receptor A antagonist including but not limited to ZD-4054; an anti-CTLA4 inhibitor including but not limited to MDX-010 (ipilimumab); an heat shock protein 27 (HSP27) inhibitor including but not limited to OGX 427; an androgen receptor degrader including but not limited to ARV-330, ARV-110; a androgen receptor DNA-binding domain inhibitor including but not limited to VPC-14449; a bromodomain and extra-terminal motif (BET) inhibitor including but not limited to BI-894999, GSK25762, GS-5829; an androgen receptor N-terminal domain inhibitor including but not limited to a sintokamide; an alpha-particle emitting radioactive therapeutic agent including but not limited to radium 233 or a salt thereof, niclosamide; or related compounds thereof, a selective estrogen receptor modulator (SERM) including but not limited to tamoxifen, raloxifene, toremifene, arzoxifene, bazedoxifene, pipindoxifene, lasofoxifene, enclomiphene; a selective estrogen receptor degrader (SERD) including but not limited to fulvestrant, ZB716, OP-1074, elacestrant, AZD9496, GDC0810, GDC0927, GW5638, GW7604; an aromitase inhibitor including but not limited to anastrazole, exemestane, letrozole; selective progesterone receptor modulators (SPRM) including but not limited to mifepristone, lonaprison, onapristone, asoprisnil, lonaprisnil, ulipristal, telapristone; a glucocorticoid receptor inhibitor including but not limited to mifepristone, COR108297, COR125281, ORIC-101, PT150; CDK4/6 inhibitors including palbociclib, abemaciclib, ribociclib; HER2 receptor antagonist including but not limited to trastuzumab, neratinib; a mammalian target of rapamycin (mTOR) inhibitor including but not limited to everolimus, temsirolimus.

In a further embodiment of the present disclosure, a pharmaceutical composition comprising one or more compounds of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt or solvate thereof, and a pharmaceutically acceptable excipient or adjuvant is provided. The pharmaceutically acceptable excipients and adjuvants are added to the composition or formulation for a variety of purposes. In another embodiment, a pharmaceutical composition comprising one or more compounds of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt or solvate thereof, further comprises a pharmaceutically acceptable carrier. In one embodiment, a pharmaceutically acceptable carrier includes a pharmaceutically acceptable excipient, binder, and/or diluent. In one embodiment, suitable pharmaceutically acceptable excipients include, but are not limited to, water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, the pharmaceutical compositions of the present disclosure may additionally contain other adjunct components conventionally found in pharmaceutical compositions, at their art-established usage levels. Thus, for example, the pharmaceutical compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The compounds disclosed herein can be formulated in accordance with the routine procedures adapted for desired administration route. Accordingly, the compounds disclosed herein can take such forms as suspensions, dispersions, solutions or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, Pa.

In certain embodiments, a pharmaceutical composition of the present disclosure is prepared using known techniques, including, but not limited to mixing, dissolving, granulating, dragee-making, levigating, emulsifying, encapsulating, entrapping or tableting processes.

In one embodiment, the present disclosure provides a pharmaceutical composition comprising a compound of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt or solvate thereof, as disclosed herein, combined with a pharmaceutically acceptable carrier. In one embodiment, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 to about 0.1 M and preferably 0.05M phosphate buffer or 0.8% saline. Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the present application include, but are not limited to, inert substances such as lactose, starch, glucose, methylcellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and may make a pharmaceutical dosage form containing the composition easier for the patient and care giver to handle. Diluents for solid compositions include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT(r)), potassium chloride, powdered cellulose, sodium chloride, sorbitol, and talc.

Additional embodiments relate to the pharmaceutical formulations wherein the formulation is selected from the group consisting of a solid, powder, liquid and a gel. In certain embodiments, a pharmaceutical composition of the present invention is a solid (e.g., a powder, tablet, a capsule, granulates, and/or aggregates). In certain of such embodiments, a solid pharmaceutical composition comprising one or more ingredients known in the art, including, but not limited to, starches, sugars, diluents, granulating agents, lubricants, binders, and disintegrating agents.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g., carbopol), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL), hydroxypropyl methyl cellulose (e.g., METHOCEL), liquid glucose, magnesium aluminum silicate, maltodextrin, methylcellulose, polymethacrylates, povidone (e.g., KOLLIDON, PLASDONE), pregelatinized starch, sodium alginate, and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g., AC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB), potato starch, and starch.

Glidants can be added to improve the flowability of a non-compacted solid composition and to improve the accuracy of dosing. Excipients that may function as glidants include silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In certain embodiments, a pharmaceutical composition of the present invention is a liquid (e.g., a suspension, elixir and/or solution). In certain of such embodiments, a liquid pharmaceutical composition is prepared using ingredients known in the art, including, but not limited to, water, glycols, oils, alcohols, flavoring agents, preservatives, and coloring agents.

Liquid pharmaceutical compositions can be prepared using compounds of formula (I), (I-A), (I-B), (II), (A), (A-I), (A-II), (A-III), (B), (B-I), (C), (D), (E), (E-I), (F), (G), (H), (J), and (K), or a pharmaceutically acceptable salt or solvate thereof, and any other solid excipients where the components are dissolved or suspended in a liquid carrier such as water, vegetable oil, alcohol, polyethylene glycol, propylene glycol, or glycerin.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Liquid pharmaceutical compositions can contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as aspartame, lactose, sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated hydroxyanisole, and ethylenediamine tetraacetic acid may be added at levels safe for ingestion to improve storage stability.

A liquid composition can also contain a buffer such as guconic acid, lactic acid, citric acid or acetic acid, sodium guconate, sodium lactate, sodium citrate, or sodium acetate. Selection of excipients and the amounts used may be readily determined by the formulation scientist based upon experience and consideration of standard procedures and reference works in the field.

In one embodiment, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables. Formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

In certain embodiments, a pharmaceutical composition of the present invention is formulated as a depot preparation. Certain such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition of the present invention comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition of the present invention comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80 and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition of the present invention comprises a sustained-release system. A non-limiting example of such a sustained-release system is a semi-permeable matrix of solid hydrophobic polymers. In certain embodiments, sustained-release systems may, depending on their chemical nature, release pharmaceutical agents over a period of hours, days, weeks or months.

Appropriate pharmaceutical compositions of the present disclosure can be determined according to any clinically-acceptable route of administration of the composition to the subject. The manner in which the composition is administered is dependent, in part, upon the cause and/or location. One skilled in the art will recognize the advantages of certain routes of administration. The method includes administering an effective amount of the agent or compound (or composition comprising the agent or compound) to achieve a desired biological response, e.g., an amount effective to alleviate, ameliorate, or prevent, in whole or in part, a symptom of a condition to be treated, e.g., oncology and neurology disorders. In various aspects, the route of administration is systemic, e.g., oral or by injection. The agents or compounds, or pharmaceutically acceptable salts or derivatives thereof, are administered orally, nasally, transdermally, pulmonary, inhalationally, buccally, sublingually, intraperintoneally, subcutaneously, intramuscularly, intravenously, rectally, intrapleurally, intrathecally, intraportally, and parenterally. Alternatively, or in addition, the route of administration is local, e.g., topical, intra-tumor and peri-tumor. In some embodiments, the compound is administered orally.

In certain embodiments, a pharmaceutical composition of the present disclosure is prepared for oral administration. In certain of such embodiments, a pharmaceutical composition is formulated by combining one or more agents and pharmaceutically acceptable carriers. Certain of such carriers enable pharmaceutical compositions to be formulated as tablets, pills, dragees, capsules, liquids, gels, syrups, slurries, suspensions and the like, for oral ingestion by a subject. Suitable excipients include, but are not limited to, fillers, such as sugars, including lactose, lactose monohydrate, sucrose, mannitol, or sorbitol; cellulose preparations such as, for example, maize starch, wheat starch, rice starch, potato starch, gelatin, gum tragacanth, methyl cellulose, microcrystalline cellulose, hydroxypropylmethyl-cellulose, sodium carboxymethylcellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross-linked polyvinyl pyrrolidone, agar, or alginic acid or a salt thereof, such as sodium alginate) are added.

In certain embodiments, dragee cores are provided with coatings. In certain such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, pharmaceutical compositions for oral administration are push-fit capsules made of gelatin. Certain of such push-fit capsules comprise one or more pharmaceutical agents of the present invention in admixture with one or more filler such as lactose, binders such as starches, and/or lubricants such as talc or magnesium stearate and, optionally, stabilizers. In certain embodiments, pharmaceutical compositions for oral administration are soft, sealed capsules made of gelatin and a plasticizer, such as glycerol or sorbitol. In certain soft capsules, one or more pharmaceutical agents of the present invention are be dissolved or suspended in suitable liquids, such as fatty oils, liquid paraffin, or liquid polyethylene glycols. In addition, stabilizers may be added.

In certain embodiments, pharmaceutical compositions are prepared for buccal administration. Certain of such pharmaceutical compositions are tablets or lozenges formulated in conventional manner.

In certain embodiments, a pharmaceutical composition is prepared for transmucosal administration. In one embodiment, penetrants appropriate to the barrier to be permeated are used in the formulation. Such penetrants are generally known in the art.

In certain embodiments, a pharmaceutical composition is prepared for administration by inhalation. Certain of such pharmaceutical compositions for inhalation are prepared in the form of an aerosol spray in a pressurized pack or a nebulizer. Certain of such pharmaceutical compositions comprise a propellant, e.g., dichlorodifluoromethane, trichlorofluoromethane, dichlorotetrafluoroethane, carbon dioxide or other suitable gas. In certain embodiments using a pressurized aerosol, the dosage unit may be determined with a valve that delivers a metered amount. In certain embodiments, capsules and cartridges for use in an inhaler or insufflator may be formulated. Certain of such formulations comprise a powder mixture of a pharmaceutical agent of the invention and a suitable powder base such as lactose or starch.

In other embodiments the compound of the present disclosure are administered by the intravenous route. In further embodiments, the parenteral administration may be provided in a bolus or by infusion.

In certain embodiments, a pharmaceutical composition is prepared for rectal administration, such as a suppository or retention enema. Certain of such pharmaceutical compositions comprise known ingredients, such as cocoa butter and/or other glycerides.

In certain embodiments, a pharmaceutical composition is prepared for topical administration. Certain of such pharmaceutical compositions comprise bland moisturizing bases, such as ointments or creams. Exemplary suitable ointment bases include, but are not limited to, petrolatum, petrolatum plus volatile silicones, and lanolin and water in oil emulsions. Exemplary suitable cream bases include, but are not limited to, cold cream and hydrophilic ointment.

In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

In certain embodiments, one or more compounds of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt or solvate thereof are formulated as a prodrug. In certain embodiments, upon in vivo administration, a prodrug is chemically converted to the biologically, pharmaceutically or therapeutically more active form. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid upon administration. In certain instances, the carboxylic acid containing compound is the corresponding active form. In certain embodiments, a prodrug comprises a short peptide (polyaminoacid) bound to an acid group. In certain of such embodiments, the peptide is cleaved upon administration to form the corresponding active form.

In certain embodiments, a prodrug is produced by modifying a pharmaceutically active compound such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

In various aspects, the amount of the compound of formula (I), (I-A), (I-B), (II), (A), (A-I), (A-II), (A-III), (B), (B-I), (C), (D), (E), (E-I), (F), (G), (H), (J), and/or (K), or a pharmaceutically acceptable salt or solvate thereof, or compounds disclosed in Table A, or a pharmaceutically acceptable salt or solvate thereof, can be administered at about 0.001 mg/kg to about 100 mg/kg body weight (e.g., about 0.01 mg/kg to about 10 mg/kg or about 0.1 mg/kg to about 5 mg/kg).

The concentration of a disclosed compound in a pharmaceutically acceptable mixture will vary depending on several factors, including the dosage of the compound to be administered, the pharmacokinetic characteristics of the compound(s) employed, and the route of administration. The agent may be administered in a single dose or in repeat doses. The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. Treatments may be administered daily or more frequently depending upon a number of factors, including the overall health of a patient, and the formulation and route of administration of the selected compound(s). An ordinarily skilled physician or veterinarian can readily determine and prescribe the effective amount of the drug required to prevent, counter or arrest the progress of the condition.

The compounds or pharmaceutical compositions of the present disclosure may be manufactured and/or administered in single or multiple unit dose forms.

Having now generally described the invention, the same will be more readily understood through reference to the following examples, which are provided by way of illustration and are not intended to be limiting of the present invention.

### EXAMPLES

The disclosure now being generally described, it will be more readily understood by reference to the following examples which are included merely for purposes of illustration of certain aspects and embodiments of the present invention and are not intended to limit the invention.

Synthetic Preparation

The novel compounds of the present invention can be prepared in a variety of ways known to one skilled in the art of organic synthesis. The compounds of the present invention can be synthesized using the methods as hereinafter described below, together with synthetic methods known in the art of synthetic organic chemistry or variations thereon as appreciated by those skilled in the art.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene and Wuts, Protective Groups in Organic Synthesis, 44th. Ed., Wiley & Sons, 2006, as well as in Jerry March, Advanced Organic Chemistry, 4^{th} edition, John Wiley & Sons, publisher, New York, 1992 which are incorporated herein by reference in their entirety.

Compounds of the present invention can be prepared by the literature methods cited in the following text. The following schemes depict established, known syntheses of these scaffolds.

The groups and/or the substituents of the compounds of the present invention can be synthesized and attached to these scaffolds by the literature methods cited in the following text. The following schemes depict the known techniques for accomplishing this joinder.

General Synthesis

Compounds of the present invention can be synthesized using the following methods. General reaction conditions are given, and reaction products can be purified by general known methods including crystallization, silica gel chromatography using various organic solvents such as hexane, cyclohexane, ethyl acetate, methanol and the like, preparative high pressure liquid chromatography or preparative reverse phase high pressure liquid chromatography.

Representative Synthesis

### Example 1: Synthesis of N-(4-((4-(2-(2,4′-dichloro-6-cyano-[1,1′-biphenyl]-4-yl)propan-2-yl) phenoxy)methyl)pyrimidin-2-yl)methanesulfonamide (Compound A2)

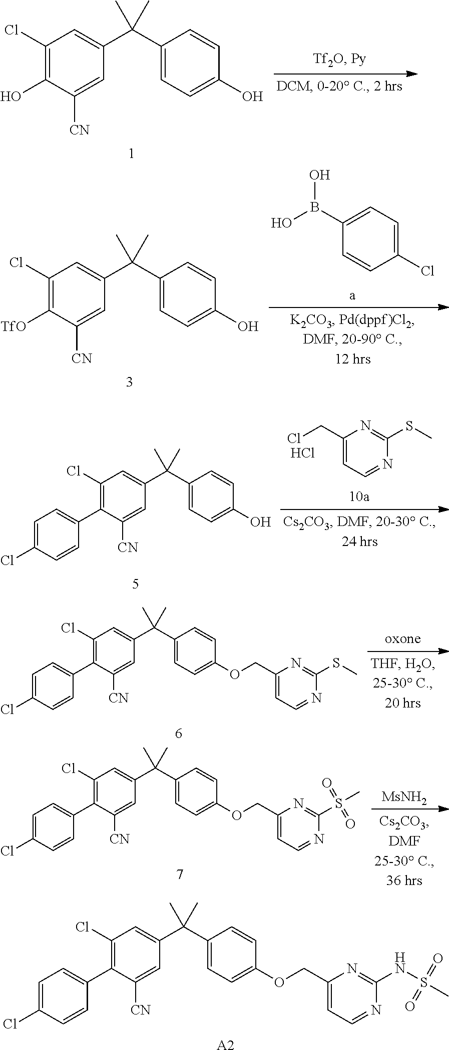

2-chloro-6-cyano-4-(2-(4-hydroxyphenyl)propan-2-yl)phenyltrifluoromethanesulfonate (3): To a solution of 3-chloro-2-hydroxy-5-(2-(4-hydroxyphenyl)propan-2-yl) benzonitrile (1) (4 g, 13.9 mmol), pyridine (3.30 g, 41.7 mol) in DCM (40 mL) was added trifluoromethanesulfonic anhydride (2.75 g, 9.73 mmol) at 0° C. Then the mixture was stirred at 20° C. for 2 hrs. TLC showed the reaction was completed. The mixture was poured into H₂O (40 mL) and extracted with DCM (20 mL×3). The organic layers were combined and washed with brine (40 mL×2), dried over Na₂SO₄, filtered and concentrated under reduced pressure. The residue was purified by silica gel column chromatography to give 2-chloro-6-cyano-4-(2-(4-hydroxyphenyl) propan-2-yl)phenyltrifluoromethanesulfonate (2.9 g, yield: 49.7%) as yellow oil. ¹H NMR (400 MHz, DMSO-d₆) δ 9.33 (s, 1H), 7.94 (d, J=2.4 Hz, 1H), 7.84 (d, J=2.4 Hz, 1H), 7.06 (d, J=8.8 Hz, 2H), 6.70 (d, J=8.8 Hz, 2H), 1.64 (s, 6H).

4′,6-dichloro-4-(2-(4-hydroxyphenyl)propan-2-yl)-[1,1′-biphenyl]-2-carbonitrile (5): To a solution of 2-chloro-6-cyano-4-(2-(4-hydroxyphenyl)propan-2-yl)phenyl trifluoromethanesulfonate (3) (500 mg, 1.19 mmol) in DMF (10 mL) was added (4-chlorophenyl)boronic acid (a) (279 mg, 1.79 mmol), K₂CO₃ (329 mg, 2.38 mmol) and Pd(dppf)Cl₂ (87.1 mg, 0.119 mmol) under N₂ atmosphere at 20° C. Then the resulting mixture was stirred at 90° C. for 12 hrs. LCMS showed the reaction was completed. The mixture was poured into H₂O (20 mL) and extracted with EtOAc (10 mL×3). The organic layers were washed with brine (10 mL), dried over Na₂SO₄, filtered and concentrated under reduced pressure. The residue was purified by silica gel column chromatography to give 4′,6-dichloro-4-(2-(4-hydroxyphenyl)propan-2-yl)-[1,1′-biphenyl]-2-carbonitrile (180 mg, yield: 35.6%) as brown solid. ¹H NMR (400 MHz, DMSO-d₆) δ 11.04 (s, 1H), 7.70-7.65 (m, 2H), 7.60 (d, J=8.8 Hz, 2H), 7.53-7.45 (m, 4H), 7.32 (d, J=8.4 Hz, 2H), 1.67-1.63 (m, 6H).

4′,6-dichloro-4-(2-(4-((2-(methylthio)pyrimidin-4-yl)methoxy)phenyl)propan-2-yl)-[1,1′-biphenyl]-2-carbonitrile (6): To a solution of 4′,6-dichloro-4-(2-(4-hydroxyphenyl)propan-2-yl)-[1,1′-biphenyl]-2-carbonitrile (5) (180 mg, 471 umol) in DMF (2 mL) was added 4-(chloromethyl)-2-(methylthio) pyrimidine (10a) (166 mg, 0.706 mmol) and Cs₂CO₃ (384 mg, 1.18 mmol) at 20° C. The mixture was stirred at 30° C. for 24 hrs. LCMS showed the reaction was completed. The mixture was poured into H₂O (10 mL) and extracted with EtOAc (10 mL×3). The organic layers were washed with brine (20 mL×4), dried over Na₂SO₄, filtered and concentrated under reduced pressure. The residue was purified by silica gel column chromatography to give 4′,6-dichloro-4-(2-(4-((2-(methylthio)pyrimidin-4-yl) methoxy)phenyl)propan-2-yl)-[1,1′-biphenyl]-2-carbonitrile (80 mg, yield: 29.4%) as yellow solid. ¹H NMR (400 MHz, DMSO-d₆) δ. 8.73 (d, J=5.2 Hz, 1H), 7.74-7.66 (m, 4H), 7.62 (d, J=8.4 Hz, 2H), 7.53-7.48 (m, 2H), 7.45 (d, J=14.8 Hz, 1H), 7.35 (d, J=8.4 Hz, 2H), 5.26 (s, 2H), 2.45 (s, 3H), 1.70 (s, 6H).

4′,6-dichloro-4-(2-(4-((2-(methylsulfonyl)pyrimidin-4-yl)methoxy)phenyl)propan-2-yl)-[1,1′-biphenyl]-2-carbonitrile (7): To a solution of 4′,6-dichloro-4-(2-(4-((2-(methylthio)pyrimidin-4-yl)methoxy)phenyl)propan-2-yl)-[1,1′-biphenyl]-2-carbonitrile (6) (80 mg, 0.138 mmol) in THF/H₂O (2 mL, v:v=1:1) was added Oxone (298 mg, 484 umol) at 25° C. The mixture was stirred at 30° C. for 20 hrs. LCMS showed the reaction was completed. The mixture was poured into H₂O (5 mL) and extracted with EtOAc (5 mL×3). The organic layers were washed with brine (5 mL), dried over Na₂SO₄, filtered and concentrated under reduced pressure to give 4′,6-dichloro-4-(2-(4-((2-(methylsulfonyl)pyrimidin-4-yl)methoxy)phenyl)propan-2-yl)-[1,1′-biphenyl]-2-carbonitrile (80 mg, yield: 94.2%) as yellow solid. ¹H NMR (400 MHz, DMSO-d₆) δ. 9.20 (d, J=5.2 Hz, 1H), 8.11 (d, J=5.2 Hz, 1H), 7.75 (d, J=2.0 Hz, 1H), 7.70-7.68 (m, 3H), 7.62 (d, J=8.4 Hz, 2H), 7.50 (d, J=8.4 Hz, 2H), 7.36 (d, J=8.4 Hz, 2H), 5.48 (s, 2H), 3.39 (s, 3H), 1.70 (s, 6H).

N-(4-((4-(2-(2,4′-dichloro-6-cyano-[1,1′-biphenyl]-4-yl)propan-2-yl)phenoxy)methyl)pyrimidin-2-yl)methanesulfonamide (A2): To a solution of 4′,6-dichloro-4-(2-(4-((2-(methylsulfonyl)pyrimidin-4-yl)methoxy)phenyl)propan-2-yl)-[1,1′-biphenyl]-2-carbonitrile (7) (80 mg, 130 umol) in DMF (2 mL) was added methanesulfonamide (55.8 mg, 586 umol) and Cs₂CO₃ (149 mg, 0.456 mmol) at 25° C., The mixture was stirred at 30° C. for 36 hrs. LCMS showed the reaction was completed. The mixture was poured into H₂O (2 mL) and used aq. HCl (3 M) to pH=4-5, then extracted with EtOAc (5 mL×3). The organic layers were washed with brine (5 mL×2), dried over Na₂SO₄, filtered and concentrated under reduced pressure. The residue was purified by prep-HPLC (FA) to give N-(4-((4-(2-(2,4′-dichloro-6-cyano-[1,1′-biphenyl]-4-yl)propan-2-yl)phenoxy)methyl)pyrimidin-2-yl)methanesulfonamide (23.2 mg, yield: 29.8%) as yellow solid. ¹H NMR (400 MHz, DMSO/NH₃.H₂O δ 8.18 (d, J=5.2 Hz, 1H), 7.86 (d, J=1.2 Hz, 1H), 7.64 (d, J=8.4 Hz, 2H), 7.56 (d, J=8.4 Hz, 2H), 7.46 (d, J=8.4 Hz, 2H), 7.31 (d, J=8.4 Hz, 2H), 7.16 (d, J=1.6 Hz, 1H), 6.75 (s, 2H), 6.61 (d, J=5.2 Hz, 1H), 2.81 (S, 3H), 1.71 (s, 6H). LCMS (220 nm): 97.911%. Exact Mass: 566.1; found 567.0/569.0.

### Example 2: Synthesis of N-(4-((4-(2-(2,3′-dichloro-6-cyano-[1,1′-biphenyl]-4-yl)propan-2-yl) phenoxy)methyl)pyrimidin-2-yl)methanesulfonamide (Compound A1)

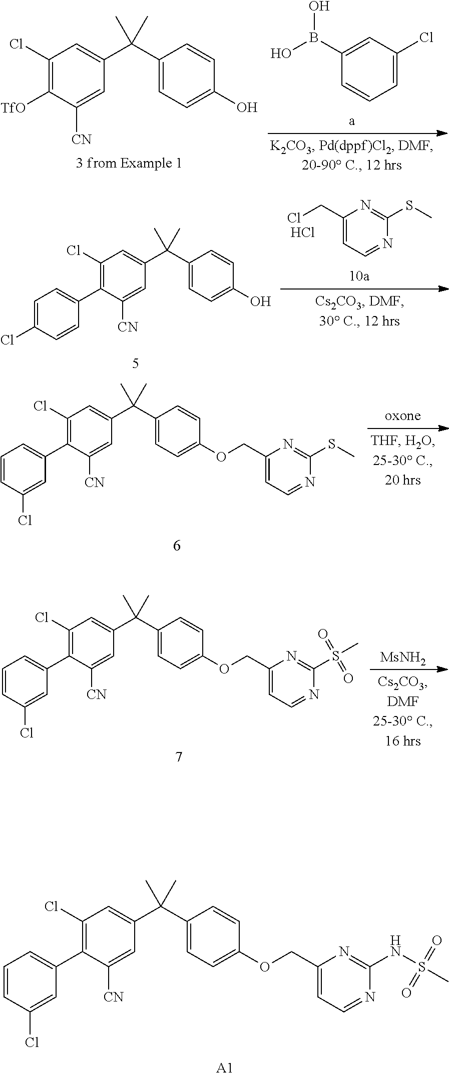

3′,6-dichloro-4-(2-(4-hydroxyphenyl)propan-2-yl)-[1,1′-biphenyl]-2-carbonitrile (5): To a solution of 2-chloro-6-cyano-4-(2-(4-hydroxyphenyl)propan-2-yl)phenyl trifluoromethanesulfonate (3) (900 mg, 2.14 mmol) in DMF (10 mL) was added (3-chlorophenyl)boronic acid (a) (670 mg, 4.29 mmol), K₂CO₃ (593 mg, 4.29 mmol) and Pd(dppf)Cl₂ (157 mg, 214 μmol) under N₂ atmosphere at 20° C. Then the resulting mixture was stirred at 90° C. for 12 hrs. LCMS showed the reaction was completed. The mixture was poured into H₂O (20 mL) and extracted with EtOAc (10 mL×3). The organic layers were washed with brine (10 mL), dried over Na₂SO₄, filtered and concentrated under reduced pressure. The residue was purified by silica gel column chromatography to give 3′,6-dichloro-4-(2-(4-hydroxyphenyl)propan-2-yl)-[1,1′-biphenyl]-2-carbonitrile (240 mg, yield: 26.4%) as brown solid. ¹H NMR (400 MHz, DMSO-d₆) δ 11.05 (s, 1H), 7.70 (t, J=1.6 Hz, 1H), 7.65-7.61 (m, 2H), 7.53-7.49 (m, 1H), 7.48-7.45 (m, 2H), 7.43-7.39 (m, 2H), 7.33 (d, J=8.4 Hz, 2H), 1.67-1.63 (m, 6H).

4′,6-dichloro-4-(2-(4-((2-(methylthio)pyrimidin-4-yl)methoxy)phenyl)propan-2-yl)-[1,1′-biphenyl]-2-carbonitrile (6): To a solution of 3′,6-dichloro-4-(2-(4-hydroxyphenyl) propan-2-yl)-[1,1′-biphenyl]-2-carbonitrile (5) (240 mg, 0.628 mmol) in DMF (3 mL) was added 4-(chloromethyl)-2-(methylthio)pyrimidine (10a) (294 mg, 0.126 mmol) and Cs₂CO₃ (716 mg, 2.20 mmol) at 30° C. The mixture was stirred at 30° C. for 12 hrs. LCMS showed the reaction was completed. The mixture was poured into H₂O (20 mL) and extracted with EtOAc (10 mL×3). The organic layers were washed with brine (20 mL×4), dried over Na₂SO₄, filtered and concentrated under reduced pressure. The residue was purified by silica gel column chromatography to give 3′,6-dichloro-4-(2-(4-((2-(methylthio)pyrimidin-4-yl)methoxy)phenyl)propan-2-yl)-[1,1′-biphenyl]-2-carbonitrile (230 mg, yield: 63.3%) as yellow solid. ¹H NMR (400 MHz, DMSO-d₆) δ. 8.73 (d, J=5.2 Hz, 1H), 7.75-7.70 (m, 2H), 7.70-7.61 (m, 4H), 7.52-7.45 (m, 2H), 7.44-7.39 (m, 2H), 7.36 (d, J=8.4 Hz, 2H), 5.27 (s, 2H), 2.45 (s, 3H), 1.70 (s, 6H).

3′,6-dichloro-4-(2-(4-((2-(methylsulfonyl)pyrimidin-4-yl)methoxy)phenyl)propan-2-yl)-[1,1′-biphenyl]-2-carbonitrile (7): To a solution of 3′,6-dichloro-4-(2-(4-((2-(methylthio)pyrimidin-4-yl)methoxy)phenyl)propan-2-yl)-[1,1′-biphenyl]-2-carbonitrile (6) (230 mg, 0.442 mmol) in THF/H₂O (3 mL, v:v=1:1) was added Oxone (815 mg, 1.33 mmol) at 25° C. The mixture was stirred at 30° C. for 20 hrs. LCMS showed the reaction was completed. The mixture was poured into H₂O (10 mL) and extracted with EtOAc (10 mL×3). The organic layers were washed with brine (5 mL), dried over Na₂SO₄, filtered and concentrated under reduced pressure to give 3′,6-dichloro-4-(2-(4-((2-(methylsulfonyl)pyrimidin-4-yl)methoxy)phenyl)propan-2-yl)-[1,1′-biphenyl]-2-carbonitrile (200 mg, yield: 73.7%) as yellow solid. ¹H NMR (400 MHz, DMSO-d₆) δ. 9.20 (d, J=5.2 Hz, 1H), 8.12 (d, J=5.2 Hz, 1H), 7.77-7.76 (m, 1H), 7.73-7.70 (m, 2H), 7.69-7.61 (m, 3H), 7.52-7.46 (m, 1H), 7.45-7.34 (m, 3H), 5.48 (s, 2H), 3.41-3.37 (m, 3H), 1.77-1.66 (m, 6H).

3′,6-dichloro-4-(2-(4-((2-(methylsulfonyl)pyrimidin-4-yl)methoxy)phenyl)propan-2-yl)-[1,1′-biphenyl]-2-carbonitrile (A1): A solution of 4′,6-dichloro-4-(2-(4-((2-(methylsulfonyl)pyrimidin-4-yl)methoxy)phenyl)propan-2-yl)-[1,1′-biphenyl]-2-carbonitrile (7) (200 mg, 0.362 mmol) in DMF (3 mL) was added methanesulfonamide (86.1 mg, 0.905 mmol), Cs₂CO₃ (295 mg, 0.905 mmol) at 25° C., The mixture was stirred at 30° C. for 16 hrs. LCMS showed the reaction was completed. The mixture was poured into H₂O (5 mL) and used aq. HCl (3 M) to adjust pH=4-5, then extracted with EtOAc (10 mL×3). The organic layers were washed with brine (10 mL×2), dried over Na₂SO₄, filtered and concentrated under reduced pressure. The residue was purified by prep-HPLC (FA) to give 3′,6-dichloro-4-(2-(4-((2-(methylsulfonyl)pyrimidin-4-yl)methoxy)phenyl)propan-2-yl)-[1,1′-biphenyl]-2-carbonitrile (A1) (20.7 mg, yield: 3.63%) as yellow solid. ¹H NMR (400 MHz, DMSO-d₆/NH₃.H₂O) δ 8.18 (d, J=3.6 Hz, 1H), 7.86 (s, 1H), 7.71-7.53 (m, 5H), 7.50-7.28 (m, 5H), 7.16 (s, 1H), 6.75 (s, 2H), 6.61 (s, 1H), 2.81 (s, 3H), 1.71 (s, 6H). LCMS (220 nm, 90.133% purity), Exact Mass: 566.1; found 567.0/569.0.

### Example 3: Synthesis of N-(4-(((4-(3-chloro-4-(2-chloroethoxy)-5-cyanophenyl)-3,4-dihydro-2H-benzo[b][1,4]oxazin-7-yl)oxy)methyl)pyrimidin-2-yl)methanesulfonamide (Compound A28)

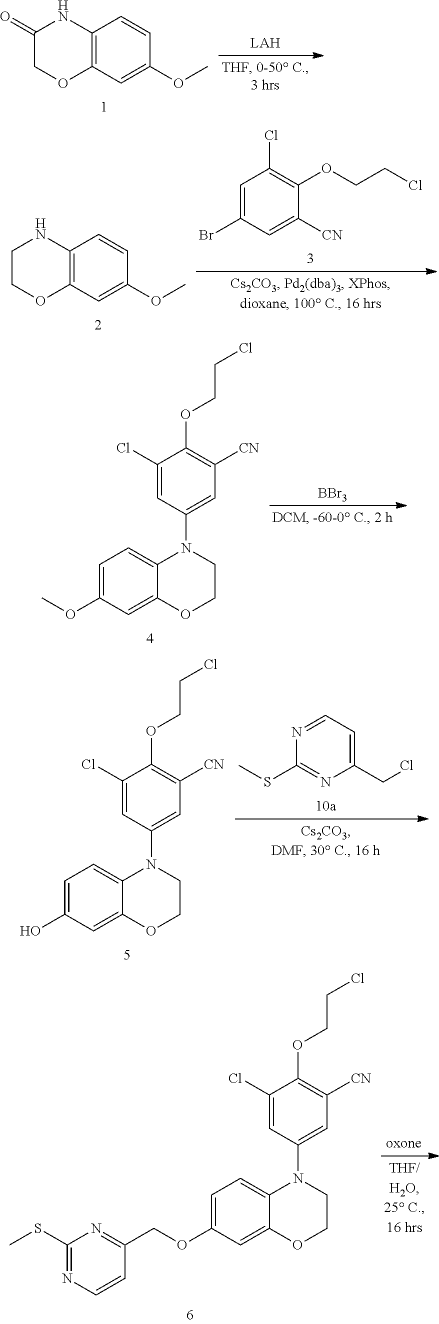

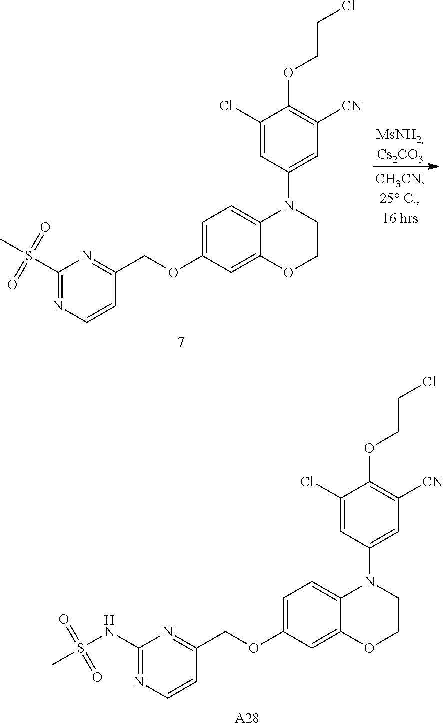

7-methoxy-3,4-dihydro-2H-benzo[b][1,4]oxazine (2): To a solution of 7-methoxy-2H-benzo[b][1,4]oxazin-3(4H)-one (1) (2 g, 10 mmol) in THE (50 mL) was added LiAlH₄ (953 mg, 25.1 mmol) at 0° C. and the mixture was stirred at 50° C. for 3 hrs under N₂ atmosphere. The mixture was quenched with water (1 mL) and the mixture was filtered, then the filtrate was dried over Na₂SO₄, filtered and concentrated under reduced pressure. The residue was purified by silica gel column to give 7-methoxy-3,4-dihydro-2H-benzo[b][1,4]oxazine (2) (1.60 g, yield: 86.8%) as yellow oil. ¹H NMR (400 MHz, CDCl₃) δ: 6.57 (d, J=8.6 Hz, 1H), 6.46-6.35 (m, 2H), 4.28-4.22 (m, 2H), 3.73 (s, 3H), 3.38 (br s, 2H).

3-chloro-2-(2-chloroethoxy)-5-(7-methoxy-2H-benzo[b][1,4]oxazin-4(3H)-yl)benzonitrile (4): To a solution of 7-methoxy-3,4-dihydro-2H-benzo[b][1,4]oxazine (0.560 g, 3.39 mmol), 5-bromo-3-chloro-2-(2-chloroethoxy)benzonitrile (1.00 g, 3.39 mmol) and Cs₂CO₃ (2.21 g, 6.78 mmol) in dioxane (10 mL) was added tris(dibenzylideneacetone)dipalladium(0) (0.311 g, 0.339 mmol) and Xantphos (0.392 g, 0.678 mmol) at 20° C. and the mixture was stirred at 100° C. for 16 hrs under N₂ atmosphere. LCMS showed the reaction was completed. The mixture was quenched with water (20 mL) and extracted with EtOAc (20 mL×3). The combined organic layers were washed with brine (50 mL), dried over Na₂SO₄, filtered and concentrated under reduced pressure. The crude was purified by silica gel column to give 3-chloro-2-(2-chloroethoxy)-5-(7-methoxy-2H-benzo[b][1,4]oxazin-4(3H)-yl)benzonitrile (1.8 g, crude) as yellow solid. ¹H NMR (400 MHz, CDCl₃) δ 7.37 (d, J=2.8 Hz, 1H), 7.22 (d, J=2.8 Hz, 1H), 6.89 (d, J=8.8 Hz, 1H), 6.52 (d, J=2.8 Hz, 1H), 6.46 (dd, J=2.8, 8.8 Hz, 1H), 4.38 (t, J=6.0 Hz, 2H), 4.21 (t, J=4.0 Hz, 2H), 3.89 (t, J=6.0 Hz, 2H), 3.78 (s, 3H), (t, J=4.0 Hz, 2H).

3-chloro-2-(2-chloroethoxy)-5-(7-hydroxy-2H-benzo[b][1,4]oxazin-4(3H)-yl)benzonitrile (5): To a solution of 3-chloro-2-(2-chloroethoxy)-5-(7-methoxy-2,3-dihydro-1,4-benzoxazin-4-yl) benzonitrile (1.3 g, 3.43 mmol) in DCM (20 mL) was added drop wise BBr₃ (0.991 mL, 10.3 mmol) in DCM (5 mL) at −60° C. and stirred at −20-0° C. for 2 hrs under N₂. LCMS showed 30% of the product was observed. The mixture was quenched with water (20 mL) and extracted with DCM (20 mL×3). The combined organic layers were washed with brine (50 mL), dried over Na₂SO₄, filtered and concentrated under reduced pressure. The crude was purified by silica gel column to give 3-chloro-2-(2-chloroethoxy)-5-(7-hydroxy-2H-benzo[b][1,4]oxazin-4(3H)-yl) benzonitrile (800 mg, yield: 57.5%) as yellow oil. ¹H NMR (400 MHz, CDCl₃) δ 7.37 (d, J=2.4 Hz, 1H), 7.22 (d, J=2.4 Hz, 1H), 6.84 (d, J=8.4 Hz, 1H), 6.47 (d, J=2.8 Hz, 1H), 6.38 (dd, J=2.8, 8.4 Hz, 1H), 4.39 (t, J=6.0 Hz, 2H), 4.22-4.17 (m, 2H), 3.89 (t, J=6.0 Hz, 2H), 3.69-3.62 (m, 2H).

3-chloro-2-(2-chloroethoxy)-5-(7-((2-(methylthio)pyrimidin-4-yl)methoxy)-2H-benzo[b][1,4]oxazin-4(3H)-yl)benzonitrile (6): To a solution of 3-chloro-2-(2-chloroethoxy)-5-(7-hydroxy-2H-benzo[b][1,4]oxazin-4(3H)-yl)benzonitrile (0.400 g, 1.10 mmol) and Cs₂CO₃ (2.14 g, 6.57 mmol) in DMF (6 mL) was added 4-(chloromethyl)-2-methylsulfanyl-pyrimidine (0.574 g, 3.29 mmol) at 20° C. and stirred at 30° C. for 16 hrs under N₂. The mixture was quenched with water (10 mL) and extracted with EtOAc (15 mL×3). The combined organic layers were washed with brine (30 mL), dried over Na₂SO₄, filtered and concentrated under reduced pressure. The crude product was purified by silica gel column to give 3-chloro-2-(2-chloroethoxy)-5-(7-((2-(methylthio)pyrimidin-4-yl)methoxy)-2H-benzo[b][1,4]oxazin-4(3H)-yl)benzonitrile (0.45 g, yield: 73.5%) as yellow solid. ¹H NMR (400 MHz, CDCl₃) δ 8.55 (d, J=5.2 Hz, 1H), 7.38 (d, J=2.8 Hz, 1H), 7.23 (d, J=2.8 Hz, 1H), 7.21 (d, J=5.2 Hz, 1H), 6.89 (d, J=8.4 Hz, 1H), 6.55 (d, J=2.8 Hz, 1H), 6.50 (dd, J=2.8, 8.8 Hz, 1H), 5.06 (s, 2H), 4.39 (t, J=6.0 Hz, 2H), 4.27-4.16 (m, 2H), 3.89 (t, J=6.0 Hz, 2H), 3.71-3.62 (m, 2H), 2.60 (s, 3H).

3-chloro-2-(2-chloroethoxy)-5-(7-((2-(methylsulfonyl)pyrimidin-4-yl)methoxy)-2H-benzo[b][1,4]oxazin-4(3H)-yl)benzonitrile (7): To a solution of 3-chloro-2-(2-chloroethoxy)-5-(7-((2-(methylthio)pyrimidin-4-yl)methoxy)-2H-benzo[b][1,4]oxazin-4(3H)-yl)benzonitrile (0.2 g, 0.397 mmol) in THE (3 mL) and H₂O (3 mL) was added Oxone (0.611 g, 0.993 mmol) at 20° C. The mixture was stirred at 30° C. for 12 hrs. LCMS showed the reaction was completed. The mixture was quenched with water (10 mL) and extracted with EtOAc (10 mL×3). The combined organic layers were washed with brine (10 mL), dried over Na₂SO₄, filtered and concentrated under reduced pressure. The crude product was purified by silica gel column to give 3-chloro-2-(2-chloroethoxy)-5-(7-((2-(methylsulfonyl)pyrimidin-4-yl)methoxy)-2H-benzo[b][1,4]oxazin-4(3H)-yl)benzonitrile (0.16 g, yield: 75.2%) as yellow solid. ¹H NMR (400 MHz, CDCl₃) δ 8.94 (d, J=4.8 Hz, 1H), 7.84 (d, J=5.6 Hz, 1H), 7.39 (d, J=2.8 Hz, 1H), 7.24 (d, J=2.8 Hz, 1H), 6.91 (d, J=9.2 Hz, 1H), 6.56 (d, J=2.8 Hz, 1H), 6.51 (dd, J=2.4, 8.8 Hz, 1H), 5.25 (s, 2H), 4.40 (t, J=6.0 Hz, 2H), 4.28-4.20 (m, 2H), 3.89 (t, J=6.0 Hz, 2H), 3.73-3.58 (m, 2H), 3.40 (s, 3H).

N-(4-(((4-(3-chloro-4-(2-chloroethoxy)-5-cyanophenyl)-3,4-dihydro-2H-benzo[b][1,4]oxazin-7-yl)oxy)methyl)pyrimidin-2-yl)methanesulfonamide (A28):

A solution of 3-chloro-2-(2-chloroethoxy)-5-(7-((2-(methylsulfonyl)pyrimidin-4-yl)methoxy)-2H-benzo[b][1,4]oxazin-4(3H)-yl)benzonitrile (A28) (160 mg, 299 umol) in MeCN (3 mL) was added methanesulfonamide (71.1 mg, 747 umol), Cs₂CO₃ (243 mg, 747 umol) at 25° C., The mixture was stirred at 30° C. for 16 hrs. LCMS showed the reaction was completed. The mixture was poured into H₂O (2 mL) and used HCl (3 M) to pH=4-5, then extracted with EtOAc (3 mL×3). The organic layers were washed with brine (3 mL×2), dried over Na₂SO₄, filtered and concentrated under reduced pressure. The residue was purified by prep-HPLC (NH₄HCO₃) to give N-(4-(((4-(3-chloro-4-(2-chloroethoxy)-5-cyanophenyl)-3,4-dihydro-2H-benzo[b][1,4]oxazin-7-yl)oxy)methyl)pyrimidin-2-yl)methanesulfonamide (18.9 mg, yield: 4.48%) as white solid. ¹H NMR (400 MHz, DMSO-d₆) δ 11.36 (s, 1H), 8.62 (d, J=5.2 Hz, 1H), 7.56 (d, J=3.2 Hz, 1H), 7.52 (d, J=2.8 Hz, 1H), 7.18 (d, J=5.2 Hz, 1H), 6.96 (d, J=8.8 Hz, 1H), 6.59 (d, J=2.8 Hz, 1H), 6.54 (dd, J=2.8, 8.8 Hz, 1H), 5.08 (s, 2H), 4.35 (t, J=4.8 Hz, 2H), 4.17 (t, J=3.6 Hz, 2H), 3.96 (t, J=5.2 Hz, 2H), 3.69 (t, J=4.0 Hz, 2H), 3.36 (s, 3H). LCMS (220 nm, 97.555% purity), Exact Mass: 549.1; found 550.0/552.0.

### Example 4: Synthesis of N-(5-(((1-(3-chloro-4-(2-chloroethoxy)-5-cyanophenyl)-1H-indol-5-yl)oxy)methyl)pyrimidin-2-yl)methanesulfonamide (Compound A29)

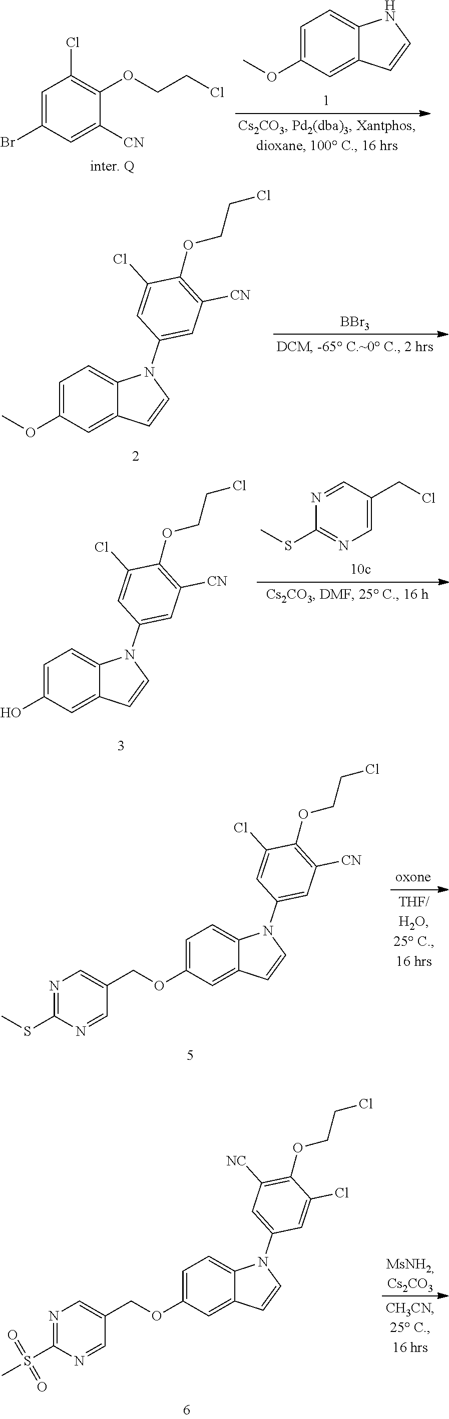

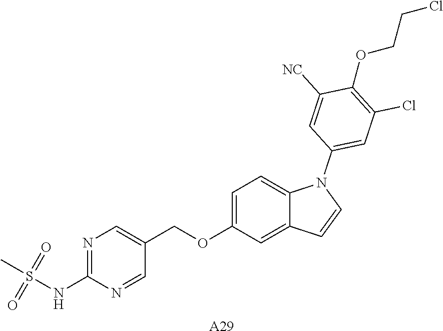 [SMILES: CS(=O)(=O)Nc1ncc(COc2ccc3c(ccn3-c3cc(Cl)c(OCCCl)c(C#N)c3)c2)cn1 ; "A29"]

3-chloro-2-(2-chloroethoxy)-5-(5-methoxy-1H-indol-1-yl)benzonitrile (2): To a solution of 5-methoxy-1H-indole (1) (0.748 g, 5.09 mmol) in dioxane (20 mL) was added 5-bromo-3-chloro-2-(2-chloroethoxy)benzonitrile (1.50 g, 5.09 mmol; intermediate Q), Tris(dibenzylideneacetone)dipalladium(0) (0.466 g, 0.509 mmol), Xantphos (0.589 g, 1.02 mmol) and Cs₂CO₃ (3.31 g, 10.2 mmol) at 20° C. and stirred at 100° C. for 16 hrs under N₂. LCMS showed the reaction was completed. The mixture was quenched with water (10 mL) and extracted with EtOAc (5 mL×3). The combined organic layers were washed with brine (10 mL), dried over Na₂SO₄, filtered and concentrated under reduced pressure. The crude was purified by silica gel column to give 3-chloro-2-(2-chloroethoxy)-5-(5-methoxyindol-1-yl)benzonitrile (2) (1.50 g, yield: 73.5%) as brown oil. ¹H NMR (400 MHz, CDCl₃) δ=7.78 (d, J=2.6 Hz, 1H), 7.64 (d, J=2.6 Hz, 1H), 7.40 (d, J=9.0 Hz, 1H), 7.23 (d, J=3.4 Hz, 1H), 7.14 (d, J=2.4 Hz, 1H), 6.94 (dd, J=2.4, 8.9 Hz, 1H), 6.66 (d, J=3.4 Hz, 1H), 4.53 (t, J=6.0 Hz, 2H), 3.95 (t, J=6.0 Hz, 2H), 3.89 (s, 3H).

3-chloro-2-(2-chloroethoxy)-5-(5-hydroxy-1H-indol-1-yl)benzonitrile (3): To a solution of 3-chloro-2-(2-chloroethoxy)-5-(5-((2-chloropyrimidin-4-yl)methoxy) indolin-1-yl)benzonitrile (0.85 g, 2.35 mmol) in DCM (20 mL) was added BBr₃ (0.907 mL, 9.41 mmol) drop wise at −60° C. and stirred at −20-0° C. for 4 hrs under N₂. The mixture was quenched with water (30 mL) and extracted with DCM (10 mL×3). The combined organic layers were washed with brine (30 mL), dried over Na₂SO₄, filtered and concentrated under reduced pressure. The crude product was purified by silica gel column to give 3-chloro-2-(2-chloroethoxy)-5-(5-hydroxy-1H-indol-1-yl) benzonitrile (200 mg, yield: 22%) as brown oil. ¹H NMR (400 MHz, CDCl₃) δ 7.81-7.74 (m, 1H), 7.69-7.58 (m, 1H), 7.40-7.33 (m, 1H), 7.25-7.20 (m, 1H), 7.13-7.06 (m, 1H), 6.93-6.79 (m, 1H), 6.67-6.57 (m, 1H), 4.78-4.59 (dr, s, 1H), 4.57-4.47 (m, 2H), 4.01-3.91 (m, 2H).

3-chloro-2-(2-chloroethoxy)-5-(5-((2-(methylthio)pyrimidin-5-yl)methoxy)-1H-indol-1-yl)benzonitrile (5): To a solution of 3-chloro-2-(2-chloroethoxy)-5-(5-hydroxyindol-1-yl)benzonitrile (4) (0.200 g, 0.576 mmol) in DMF (5 mL) was added 5-(chloromethyl)-2-methylsulfanyl-pyrimidine (10c) (0.134 g, 0.691 mmol) and Cs₂CO₃ (0.563 g, 1.73 mmol) at 20° C. The mixture was stirred at the same temperature for 12 hrs. The mixture was quenched with H₂O (10 mL). The aqueous layer was extracted with EtOAc (10 mL×3). The combined organic layers were washed with brine (20 mL×4), dried over Na₂SO₄, filtered and concentrated under reduced pressure to give a crude product. The crude product was purified by silica gel column to get 3-chloro-2-(2-chloroethoxy)-5-(5-((2-(methylthio)pyrimidin-5-yl) methoxy)-1H-indol-1-yl)benzonitrile (5) (0.246 g, yield: 79.2%) as white oil. ¹H NMR (400 MHz, CDCl₃) δ=8.64 (s, 2H), 7.78 (d, J=2.6 Hz, 1H), 7.64 (d, J=2.6 Hz, 1H), 7.41 (d, J=8.8 Hz, 1H), 7.25 (d, J=3.4 Hz, 1H), 7.20 (d, J=2.4 Hz, 1H), 6.98 (dd, J=2.4, 8.9 Hz, 1H), 6.66 (d, J=3.2 Hz, 1H), 5.07 (s, 2H), 4.57-4.47 (m, 2H), 3.95 (t, J=6.0 Hz, 2H), 2.60 (s, 3H).

3-chloro-2-(2-chloroethoxy)-5-(5-((2-(methylsulfonyl)pyrimidin-5-yl)methoxy)-1H-indol-1-yl)benzonitrile (6): To a solution of 3-chloro-2-(2-chloroethoxy)-5-(5-((2-(methylthio)pyrimidin-5-yl)methoxy)-1H-indol-1-yl)benzonitrile (5) (0.120 g, 0.247 mmol) in THE (3 mL)/H₂O (3 mL) was added Oxone (0.380 g, 0.618 mmol) at 20° C. and stirred at 20° C. for 16 hrs under N₂. The mixture was quenched with water (10 mL) and extracted with EtOAc (10 mL×3). The combined organic layers were washed with brine (20 mL), dried over Na₂SO₄, filtered and concentrated under reduced pressure. The crude was purified by p-TLC to give 3-chloro-2-(2-chloroethoxy)-5-(5-((2-(methylsulfonyl)pyrimidin-5-yl)methoxy)-1H-indol-1-yl)benzonitrile (6) (0.110 g, yield: 77.4%) as white solid. ¹H NMR (400 MHz, CDCl₃) δ=9.05 (s, 2H), 7.78 (d, J=2.6 Hz, 1H), 7.65 (d, J=2.6 Hz, 1H), 7.43 (d, J=9.0 Hz, 1H), 7.29-7.27 (m, 1H), 7.21 (d, J=2.3 Hz, 1H), 7.01 (dd, J=2.5, 9.0 Hz, 1H), 6.68 (d, J=3.3 Hz, 1H), 5.28 (s, 2H), 4.54 (t, J=6.0 Hz, 2H), 3.95 (t, J=6.0 Hz, 2H), 3.39 (s, 3H).

N-(5-(((1-(3-chloro-4-(2-chloroethoxy)-5-cyanophenyl)-1H-indol-5-yl)oxy)methyl)pyrimidin-2-yl)methanesulfonamide (A29): To a solution of 3-chloro-2-(2-chloroethoxy)-5-(5-((2-(methylsulfonyl)pyrimidin-5-yl)methoxy)-1H-indol-1-yl)benzonitrile (6) (0.100 g, 0.193 mmol) in CH₃CN (3 mL) was added MsNH₂ (0.0551 g, 0.580 mmol) and Cs₂CO₃ (0.189 g, 0.580 mmol) into the reaction at 20° C. The mixture was stirred for 16 hrs at 20° C. The mixture was quenched with H₂O (5 mL) and extracted with EtOAc (5 mL×3). The combined organic layers were washed with brine (10 mL), dried over Na₂SO₄, filtered and concentrated under reduced pressure. The residue was purified by prep-HPLC to give N-(5-(((1-(3-chloro-4-(2-chloroethoxy)-5-cyanophenyl)-1H-indol-5-yl)oxy)methyl)pyrimidin-2-yl)methanesulfonamide (A29) (0.0400 g, yield: 37.3%) as white solid. ¹H NMR (400 MHz, DMSO-d₆) δ=11.40 (br s, 1H), 8.74 (s, 2H), 8.11 (dd, J=2.6, 12.0 Hz, 2H), 7.70 (d, J=3.4 Hz, 1H), 7.55 (d, J=9.0 Hz, 1H), 7.30 (d, J=2.4 Hz, 1H), 6.95 (dd, J=2.4, 9.0 Hz, 1H), 6.67 (d, J=3.4 Hz, 1H), 5.13 (s, 2H), 4.53-4.48 (m, 2H), 4.04-4.00 (m, 2H), 3.36 (s, 3H). LCMS: (220 nm): 98.69%, Exact Mass: 531.1; found 532.1/534.0.

### Example 5: Synthesis of N-(4-(((1-(3-chloro-4-(2-chloroethoxy)-5-cyanophenyl)indolin-5-yl)oxy)methyl)pyrimidin-2-yl)methanesulfonamide (Compound A30)

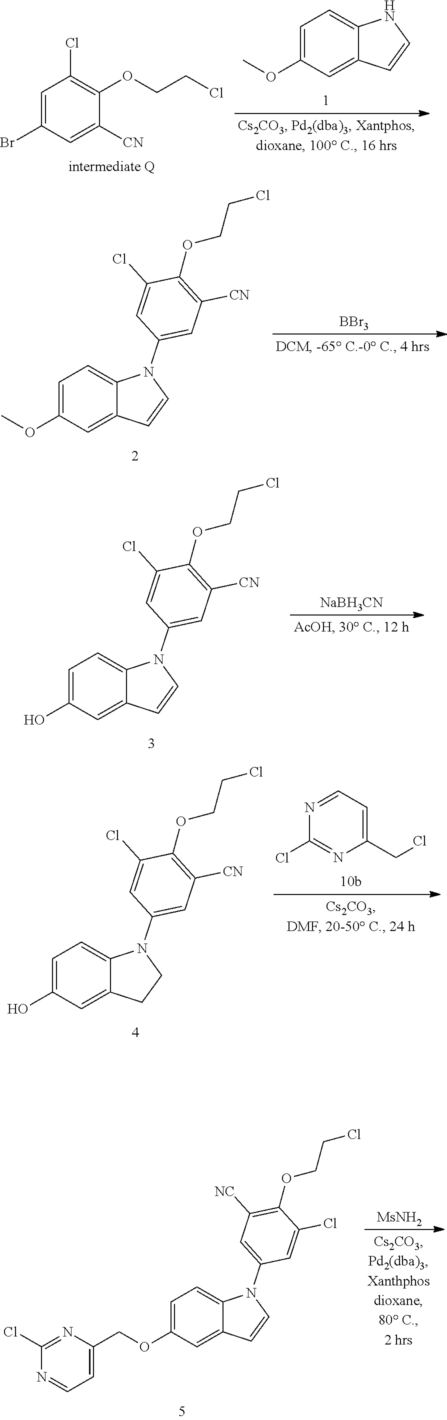

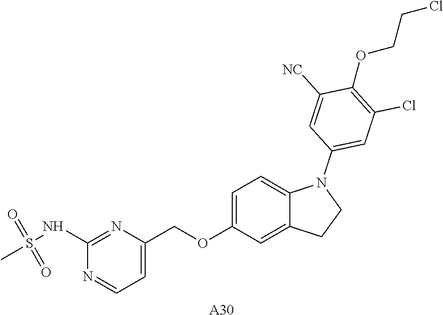 [SMILES: CS(=O)(=O)Nc1nccc(COc2ccc3c(c2)CCN3c2cc(Cl)c(OCCCl)c(C#N)c2)n1 ; "A30"]

3-chloro-2-(2-chloroethoxy)-5-(5-methoxy-1H-indol-1-yl)benzonitrile (2): To a solution of 5-methoxy-1H-indole (1) (0.748 g, 5.09 mmol) in dioxane (20 mL) was added 5-bromo-3-chloro-2-(2-chloroethoxy)benzonitrile (1.50 g, 5.09 mmol; intermediate Q), Tris(dibenzylideneacetone)dipalladium(0) (0.466 g, 0.509 mmol), Xantphos (0.589 g, 1.02 mol), and Cs₂CO₃ (3.31 g, 10.2 mmol) at 20° C. and stirred at 100° C. for 16 hrs under N₂. LCMS showed the reaction was completed. The mixture was quenched with water (10 mL) and extracted with EtOAc (20 mL×3). The combined organic layers were washed with brine (20 mL), dried over Na₂SO₄, filtered and concentrated under reduced pressure. The crude was purified by silica gel column to give 3-chloro-2-(2-chloroethoxy)-5-(5-methoxyindol-1-yl)benzonitrile (2) (1.50 g, yield: 73.5%) as brown oil. ¹H NMR (400 MHz, CDCl₃) δ=7.78 (d, J=2.6 Hz, 1H), 7.64 (d, J=2.6 Hz, 1H), 7.40 (d, J=9.0 Hz, 1H), 7.23 (d, J=3.4 Hz, 1H), 7.14 (d, J=2.4 Hz, 1H), 6.94 (dd, J=2.4, 8.9 Hz, 1H), 6.66 (d, J=3.4 Hz, 1H), 4.53 (t, J=6.0 Hz, 2H), 3.95 (t, J=6.0 Hz, 2H), 3.89 (s, 3H).

3-chloro-2-(2-chloroethoxy)-5-(5-hydroxy-1H-indol-1-yl)benzonitrile (3): To a solution of 3-chloro-2-(2-chloroethoxy)-5-(5-((2-chloropyrimidin-4-yl)methoxy)indolin-1-yl)benzonitrile (0.85 g, 2.35 mmol) in DCM (20 mL) was added BBr₃ (0.907 mL, 9.41 mmol) drop wise at −60° C. and stirred at −20-0° C. for 4 hrs under N₂. LCMS showed the reaction was completed. The mixture was quenched with water (30 mL) and extracted with DCM (10 mL×3). The combined organic layers were washed with brine (30 mL), dried over Na₂SO₄, filtered and concentrated under reduced pressure. The crude product was purified by silica gel column to give 3-chloro-2-(2-chloroethoxy)-5-(5-hydroxy-1H-indol-1-yl)benzonitrile (200 mg, yield: 22%) as brown oil. ¹H NMR (400 MHz, CDCl₃) δ 7.77 (s, 1H), 7.64 (s, 1H), 7.40-7.33 (m, 1H), 7.25-7.20 (m, 1H), 7.13-7.06 (m, 1H), 6.93-6.79 (m, 1H), 6.67-6.57 (m, 1H), 4.78-4.59 (brs, 1H), 4.57-4.47 (m, 2H), 4.01-3.91 (m, 2H).

3-chloro-2-(2-chloroethoxy)-5-(5-hydroxyindolin-1-yl)benzonitrile (4): To a solution of 3-chloro-2-(2-chloroethoxy)-5-(5-hydroxy-1H-indol-1-yl)benzonitrile (0.300 g, 8.64 mmol) in AcOH (5 mL) was added NaBH₃CN (0.271 g, 4.32 mmol) at 20° C. and was stirred at 30° C. for 12 hrs. LCMS showed the reaction was completed. The mixture was quenched with water (5 mL) and extracted with EtOAc (10 mL×3). The combined organic layers were washed with saturated NaHCO₃ (10×2 mL), brine (10 mL), dried over Na₂SO₄, filtered and concentrated under reduced pressure to give 3-chloro-2-(2-chloroethoxy)-5-(5-hydroxyindolin-1-yl)benzonitrile (0.38 g crude) as a yellow solid. ¹H NMR (400 MHz, CDCl₃) δ 7.37 (d, J=2.8 Hz, 1H), 7.22 (d, J=2.8 Hz, 1H), 6.97 (d, J=8.4 Hz, 1H), 6.79-6.76 (d, J=2.4 Hz, 1H), 6.62 (dd, J=2.4, 8.4 Hz, 1H), 4.37 (t, J=6.0 Hz, 2H), 3.95-3.85 (m, 4H), 3.12 (t, J=8.4 Hz, 2H), 2.11 (s, 2H).

3-chloro-2-(2-chloroethoxy)-5-(5-((2-chloropyrimidin-4-yl)methoxy)indolin-1-yl)benzonitrile (5): To a solution of 3-chloro-2-(2-chloroethoxy)-5-(5-hydroxyindolin-1-yl)benzonitrile (0.380 g, 1.09 mmol) and 2-chloro-4-(chloromethyl)pyrimidine (0.237 g, 1.31 mmol) in DMF (5 mL) at 20° C. was added Cs₂CO₃ (0.886 g, 2.72 mmol) at 20° C. The mixture was stirred at the same temperature for 12 hrs. After addition, the reaction was stirred at 50° C. for 12 hrs. LCMS showed the reaction was completed and the resulting mixture was quenched with H₂O (10 mL). The aqueous layer was extracted with EtOAc (10 mL×3). The combined organic layers were washed with brine (20 mL×4), dried over Na₂SO₄, filtered and concentrated under reduced pressure. The crude product under purification by column to get 3-chloro-2-(2-chloroethoxy)-5-(5-((2-chloropyrimidin-4-yl)methoxy)indolin-1-yl)benzonitrile (0.18 g, yield: 31.3%) as yellow solid. ¹H NMR (400 MHz, CDCl₃) δ 8.66 (d, J=5.2 Hz, 1H), 7.55 (d, J=4.4 Hz, 1H), 7.39 (s, 1H), 7.23 (s, 1H), 7.03 (d, J=9.2 Hz, 1H), 6.88 (s, 1H), 6.72 (d, J=8.0 Hz, 1H), 5.13 (s, 2H), 4.38 (dr, t, J=6.0 Hz, 2H), 3.97-3.87 (m, 4H), 3.16 (br t, J=8.0 Hz, 2H).

N-(4-(((1-(3-chloro-4-(2-chloroethoxy)-5-cyanophenyl)indolin-5-yl)oxy)methyl)pyrimidin-2-yl)methanesulfonamide (A30): To a solution of 3-chloro-2-(2-chloroethoxy)-5-(5-((2-chloropyrimidin-4-yl)methoxy)indolin-1-yl)benzonitrile (0.07 g, 0.147 mmol), methanesulfonamide (0.07 g, 0.736 mmol) and Cs₂CO₃ (0.0959 g, 0.294 mmol) in dioxane (3 mL) was added Tris(dibenzylideneacetone)dipalladium(0) (0.0135 g, 0.0147 mmol) and Xantphos (0.0170 g, 0.0294 mmol) at 20° C. and the mixture was stirred at 80° C. for 2 hrs under N₂ atmosphere. LCMS showed the reaction was completed. The mixture was quenched with water (5 mL) and extracted with EtOAc (5 mL×3). The combined organic layers were washed with brine (10 mL), dried over Na₂SO₄, filtered and concentrated under reduced pressure. The crude product was purified by prep-HPLC (FA) to give N-(4-(((1-(3-chloro-4-(2-chloroethoxy)-5-cyanophenyl)indolin-5-yl)oxy)methyl)pyrimidin-2-yl)methanesulfonamide (8.6 mg, yield: 13.9%) as yellow solid. ¹H NMR (400 MHz, DMSO-d₆) δ 11.39 (br s, 1H), 8.62 (d, J=4.8 Hz, 1H), 7.52 (dd, J=2.8, 14.4 Hz, 2H), 7.18 (br s, J=4.8 Hz, 1H), 7.13 (d, J=8.8 Hz, 1H), 7.02-6.98 (m, 1H), 6.76 (dd, J=2.4, 8.4 Hz, 1H), 5.09 (s, 2H), 4.35-4.31 (dr, t, J=4.8 Hz, 2H), 3.98-3.91 (m, 4H), 3.36 (s, 3H), 3.08 (br t, J=8.4 Hz, 2H). LCMS: (220 nm): 95.07%. Exact Mass: 533.1; found 534.0/536.0.

### Example 6: Synthesis of N-(4-(((3-(3-chloro-4-(2-chloroethoxy)-5-cyanophenyl)-2,3-dihydro-1H-inden-5-yl)oxy)methyl)pyrimidin-2-yl)methanesulfonamide (Compound A31)

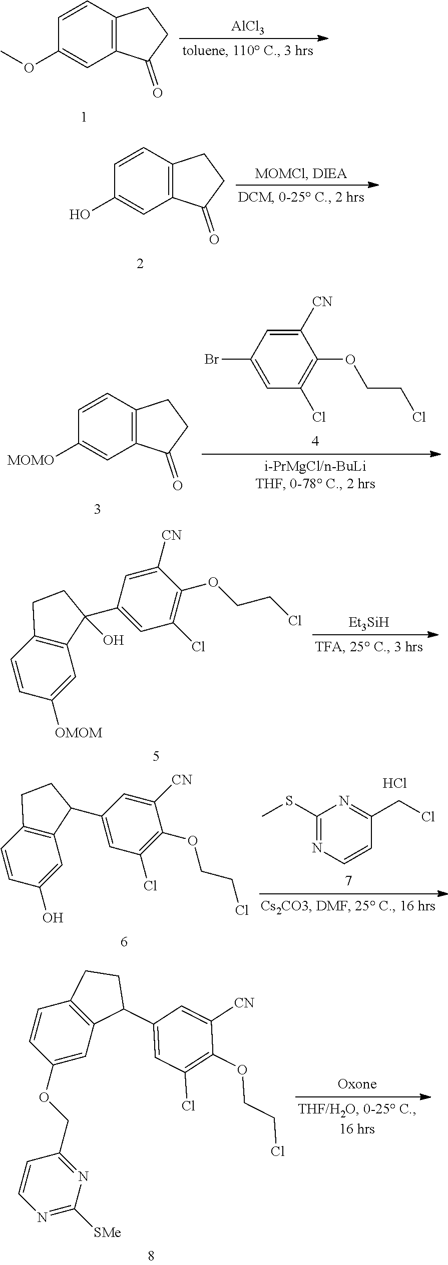

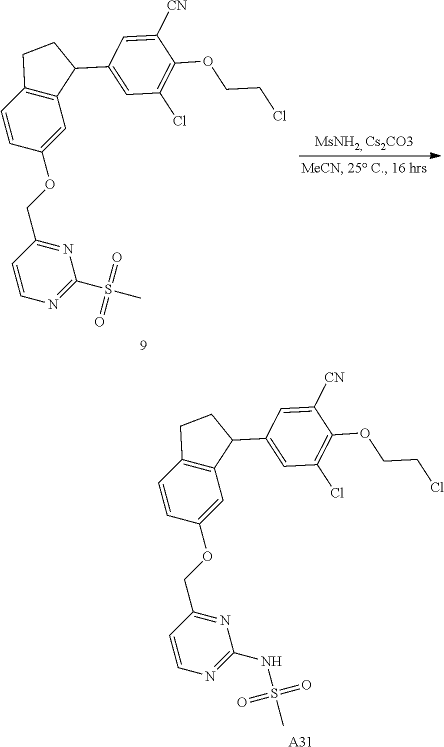

6-hydroxy-2,3-dihydro-1H-inden-1-one (2): To a solution of 6-methoxyindan-1-one (1) (15.0 g, 92.5 mmol) in toluene (150 mL) was added AICl₃ (30.8 g, 0.231 mol) at 25° C. and the mixture was stirred at 110° C. for 3 hrs. The reaction was quenched with water (300 mL). Lots of solid appeared and the mixture was filtered and the filter cake was dried under reduced pressure to give 6-hydroxy-2,3-dihydro-1H-inden-1-one (2) (8.00 g, yield: 52.5%) as brown solid. ¹H NMR (400 MHz, CDCl₃) δ=7.37 (br d, J=8.0 Hz, 1H), 7.18 (br s, 2H), 5.18 (br s, 1H), 3.13-3.03 (m, 2H), 2.79-2.68 (m, 2H).

6-(methoxymethoxy)-2,3-dihydro-1H-inden-1-one (3): To a solution of 6-hydroxy-2,3-dihydro-1H-inden-1-one (2) (10.0 g, 0.0675 mol) and DIEA (34.7 mL, 0.202 mol) in DCM (150 mL) was added chloro(methoxy)methane (10.9 g, 0.135 mol) at 0° C. and the mixture was stirred at 25° C. for 2 hrs. The mixture was quenched with water (200 mL) and extracted with DCM (100 mL×3). The combined organic layers were washed with brine (100 mL), dried over Na₂SO₄, filtered and concentrated under reduced pressure. The residue was purified by silica gel column to give 6-(methoxymethoxy)-2,3-dihydro-1H-inden-1-one (3) (8.00 g, yield: 58.6%) as yellow solid. ¹H NMR (400 MHz, CDCl₃) δ=7.43-7.37 (m, 2H), 7.31-7.27 (m, 1H), 5.20 (s, 2H), 3.48 (s, 3H), 3.14-3.04 (m, 2H), 2.76-2.67 (m, 2H).

3-chloro-2-(2-chloroethoxy)-5-(1-hydroxy-6-(methoxymethoxy)-2,3-dihydro-1H-inden-1-yl)benzonitrile (5): To n-BuLi (2500 mmol/L, 10.2 mL, 0.0254 mol) was added i-PrMgCl (2000 mmol/L, 6.36 mL, 0.0127 mol) drop wise at 0° C. under N₂ and stirred for 30 min. Then 5-bromo-3-chloro-2-(2-chloroethoxy)benzonitrile (4) (5.00 g, 0.0170 mol) in THF (50 mL) was added to the above mixture at −78° C. and stirred for 30 min under N₂ atmosphere. Then to this mixture was added 6-(methoxymethoxy)-2,3-dihydro-1H-inden-1-one (3) (3.91 g, 0.0203 mol) in THF (30 mL) drop wise at −78° C. and stirred for 2 hrs under N₂. The mixture was poured into sat. NH₄Cl (100 mL) and extracted with EtOAc (50 mL×3), the combined organic layers were washed with brine (50 mL×2), dried over Na₂SO₄, filtered and concentrated under reduced pressure. The residue was purified by p-HPLC (FA) to give 3-chloro-2-(2-chloroethoxy)-5-(1-hydroxy-6-(methoxymethoxy)-2,3-dihydro-1H-inden-1-yl)benzonitrile (5) (0.630 g, yield: 9.75%) as light oil. ¹H NMR (400 MHz, CDCl₃) δ=7.70 (d, J=2.2 Hz, 1H), 7.49 (d, J=2.2 Hz, 1H), 7.29-7.27 (m, 1H), 7.26 (s, 1H), 7.08-7.03 (m, 1H), 6.70 (d, J=2.2 Hz, 1H), 5.17-5.08 (m, 2H), 4.46 (t, J=6.2 Hz, 2H), 3.90 (t, J=6.2 Hz, 2H), 3.46 (s, 3H), 3.17-3.07 (m, 1H), 2.98-2.87 (m, 1H), 2.53-2.39 (m, 2H).

3-chloro-2-(2-chloroethoxy)-5-(6-hydroxy-2,3-dihydro-1H-inden-1-yl)benzonitrile (6): To a solution of 3-chloro-2-(2-chloroethoxy)-5-(1-hydroxy-6-(methoxymethoxy)-2,3-dihydro-1H-inden-1-yl)benzonitrile (5) (0.630 g, 1.54 mmol) in TFA (3 mL) was added (C₂H5)₃SiH (0.739 mL, 4.63 mmol) and stirred at 25° C. for 3 hrs. The mixture was poured into water (10 mL) and extracted with DCM (5 mL×3). The combined organic layers were washed with brine (5 mL×3), dried over Na₂SO₄, filtered and concentrated under reduced pressure. The residue was purified by p-TLC to give 3-chloro-2-(2-chloroethoxy)-5-(6-hydroxy-2,3-dihydro-1H-inden-1-yl)benzonitrile (6) (150 mg, yield: 25.1%) as a yellow oil. ¹H NMR (400 MHz, CDCl₃) δ=7.43 (d, J=2.2 Hz, 1H), 7.31 (d, J=2.2 Hz, 1H), 7.17 (d, J=8.2 Hz, 1H), 6.74-6.70 (m, 1H), 6.39 (d, J=1.6 Hz, 1H), 4.62 (br s, 1H), 4.44 (t, J=6.2 Hz, 2H), 4.26 (t, J=8.2 Hz, 1H), 3.90 (t, J=6.2 Hz, 2H), 3.01-2.84 (m, 2H), 2.67-2.51 (m, 1H), 2.02-1.92 (m, 1H).

3-chloro-2-(2-chloroethoxy)-5-(6-((2-(methylthio)pyrimidin-4-yl)methoxy)-2,3-dihydro-1H-inden-1-yl)benzonitrile (8): To a solution of 3-chloro-2-(2-chloroethoxy)-5-(6-hydroxy-2,3-dihydro-1H-inden-1-yl)benzonitrile (6) (150 mg, 0.43 mmol) and Cs₂CO₃ (491 mg, 1.51 mmol) in DMF (3 mL) was added 4-(chloromethyl)-2-methylsulfanyl-pyrimidine (7) (136 mg, 0.65 mmol) and stirred at 25° C. for 16 hrs. The mixture was poured into water (5 mL) and extracted with EtOAc (5 mL×3), the combined organic layers were washed with brine (5 mL×4), dried over Na₂SO₄, filtered and concentrated under reduced pressure to give a residue. The residue was purified by silica gel column to give 3-chloro-2-(2-chloroethoxy)-5-(6-((2-(methylthio)pyrimidin-4-yl)methoxy)-2,3-dihydro-1H-inden-1-yl)benzonitrile (8) (110 mg, yield: 47.2%) as a yellow oil. ¹H NMR (400 MHz, CDCl₃) δ=8.53 (br d, J=4.8 Hz, 1H), 7.41 (d, J=2.2 Hz, 1H), 7.28 (d, J=2.2 Hz, 1H), 7.25-7.19 (m, 2H), 6.87-6.82 (m, 1H), 6.50 (d, J=1.8 Hz, 1H), 5.03 (s, 2H), 4.48-4.43 (m, 2H), 4.28 (t, J=8.0 Hz, 1H), 3.95-3.87 (m, 2H), 3.05-2.87 (m, 2H), 2.68-2.53 (m, 4H), 2.00-1.95 (m, 1H).

3-chloro-2-(2-chloroethoxy)-5-(6-((2-(methylsulfonyl)pyrimidin-4-yl)methoxy)-2,3-dihydro-1H-inden-1-yl)benzonitrile (9): To a solution of 3-chloro-2-(2-chloroethoxy)-5-(6-((2-(methylthio)pyrimidin-4-yl)methoxy)-2,3-dihydro-1H-inden-1-yl)benzonitrile (8) (110 mg, 0.23 mmol) in THE (2 mL) and water (2 mL) was added Oxone (348 mg, 0.57 mmol) at 0° C. and the mixture was stirred at 25° C. for 16 hrs. The mixture was poured into water (5 mL) and extracted with EtOAc (5 mL×3), the combined organic layers were washed with brine (5 mL×2), dried over Na₂SO₄, filtered and concentrated under reduced pressure to give 3-chloro-2-(2-chloroethoxy)-5-(6-((2-(methylsulfonyl)pyrimidin-4-yl)methoxy)-2,3-dihydro-1H-inden-1-yl)benzonitrile (9) (80 mg, yield: 61.4%) as yellow oil. ¹H NMR (400 MHz, CDCl₃) δ=8.91 (d, J=5.2 Hz, 1H), 7.81 (d, J=5.2 Hz, 1H), 7.43 (d, J=2.0 Hz, 1H), 7.31-7.27 (m, 1H), 7.25 (s, 1H), 6.89-6.83 (m, 1H), 6.51 (d, J=2.0 Hz, 1H), 5.22 (s, 2H), 4.47 (t, J=6.0 Hz, 2H), 4.30 (br t, J=8.0 Hz, 1H), 3.94-3.89 (m, 2H), 3.38 (s, 3H), 3.03-2.91 (m, 2H), 2.66-2.59 (m, 1H), 2.02-1.95 (m, 1H).

N-(4-(((3-(3-chloro-4-(2-chloroethoxy)-5-cyanophenyl)-2,3-dihydro-1H-inden-5-yl)oxy) methyl)pyrimidin-2-yl)methanesulfonamide (A31): To a solution of 3-chloro-2-(2-chloroethoxy)-5-(6-((2-(methylsulfonyl)pyrimidin-4-yl)methoxy)-2,3-dihydro-1H-inden-1-yl)benzonitrile (9) (90 mg, 0.17 mmol) and MsNH₂ (41.3 mg, 0.43 mmol) in MeCN (3.00 mL) was added Cs₂CO₃ (141 mg, 0.43 mmol) at 25° C. and the mixture was stirred at 25° C. for 16 hrs. The mixture was added aq. HCl and adjust pH=6, then the mixture was poured into water (20 mL) and extracted with EtOAc (10 mL×3), the combined organic layers were washed with brine (10 mL×3), dried over Na₂SO₄, filtered and concentrated under reduced pressure to give a residue. The residue was purified by p-HPLC (FA) to give N-(4-(((3-(3-chloro-4-(2-chloroethoxy)-5-cyanophenyl)-2,3-dihydro-1H-inden-5-yl)oxy)methyl)pyrimidin-2-yl)methanesulfonamide (A31) (27.7 mg, yield: 29.6%) as white solid. ¹H NMR (400 MHz, CDCl₃) δ=8.60 (d, J=4.8 Hz, 1H), 7.43 (d, J=2.0 Hz, 1H), 7.28 (br d, J=2.0 Hz, 1H), 7.26-7.22 (m, 2H), 6.88-6.83 (m, 1H), 6.51 (s, 1H), 5.03 (s, 2H), 4.45 (t, J=6.0 Hz, 2H), 4.29 (t, J=8.4 Hz, 1H), 3.90 (t, J=6.2 Hz, 2H), 3.45 (s, 3H), 3.05-2.86 (m, 2H), 2.68-2.56 (m, 1H), 2.06-1.96 (m, 1H). LCMS: (220 nm): 99.14%. Exact Mass: 532.1; found 533.0/535.0.

### Example 7: Synthesis of N-(4-(((5-(3-chloro-4-(2-chloroethoxy)-5-cyanophenyl)-7,8-dihydronaphthalen-2-yl)oxy)methyl)pyrimidin-2-yl)methanesulfonamide (Compound A32)

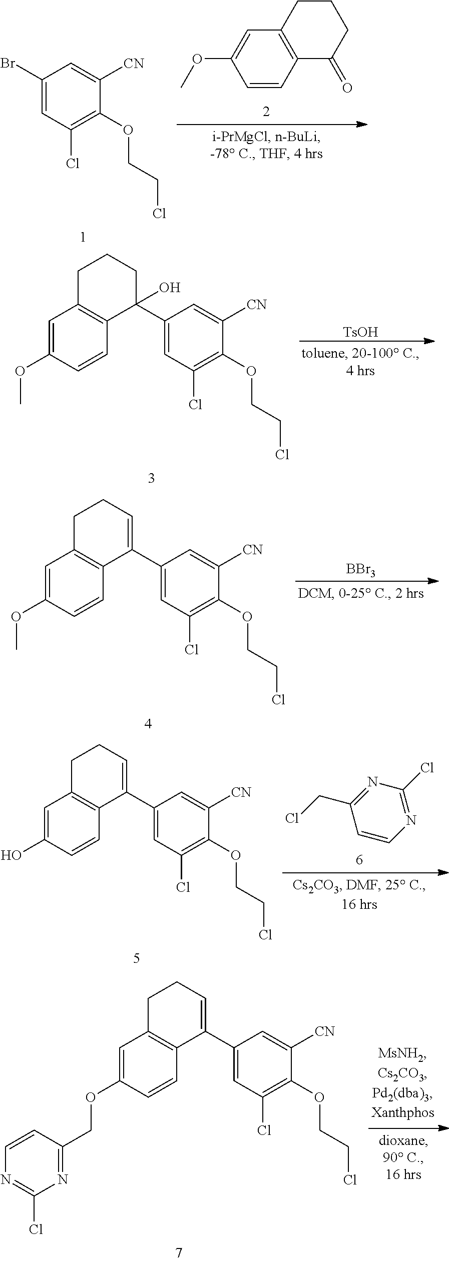

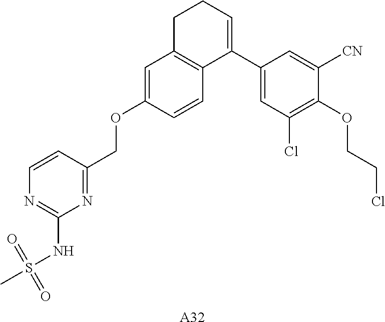 [SMILES: CS(=O)(=O)Nc1nccc(COc2ccc3c(c2)CCC=C3c2cc(Cl)c(OCCCl)c(C#N)c2)n1 ; "A32"]

3-chloro-2-(2-chloroethoxy)-5-(1-hydroxy-6-methoxy-1,2,3,4-tetrahydronaphthalen-1-yl)benzonitrile (3): To n-BuLi (2500 mmol/L, 10.2 mL, 0.0254 mol) was added i-PrMgCl (2000 mmol/L, 6.36 mL, 0.0127 mol) drop wise (lots of solid appeared) at 0° C. and stirred for 30 min. Then 5-bromo-3-chloro-2-(2-chloroethoxy)benzonitrile (1) (5.00 g, 17 mmol) in 30 mL THF was added drop wise at −78° C. and stirred for 30 min under N₂ atmosphere. Then to the mixture was added 6-methoxy-3,4-dihydronaphthalen-1(2H)-one (2) (4.48 g, 25.4 mmol) in 20 mL THF drop wise at −78° C. and the mixture was stirred at −78° C. for 3 hrs under N₂ atmosphere. The mixture was quenched with NH₄Cl (100 mL) and extracted with EtOAc (30 mL×3). The combined organic layers were washed with brine (100 mL), dried over Na₂SO₄, filtered and concentrated under reduced pressure to give 3-chloro-2-(2-chloroethoxy)-5-(1-hydroxy-6-methoxy-1,2,3,4-tetrahydronaphthalen-1-yl)benzonitrile (3) (8 g, crude) as brown oil.

3-chloro-2-(2-chloroethoxy)-5-(6-methoxy-3,4-dihydronaphthalen-1-yl)benzonitrile (4): To a solution of 3-chloro-2-(2-chloroethoxy)-5-(1-hydroxy-6-methoxy-1,2,3,4-tetrahydronaphthalen-1-yl)benzonitrile (3) (500 mg, 1.27 mmol) in toluene (8 mL) was added p-Toluenesulfonic acid (329 mg, 1.91 mmol) at 20° C. and stirred at 100° C. for 4 hrs under N₂. The mixture was quenched with water (5 mL) and extracted with EtOAc (10 mL×3). The combined organic layers were washed with brine (10 mL), dried over Na₂SO₄, filtered and concentrated under reduced pressure. The residue was purified by silica gel column to give 3-chloro-2-(2-chloroethoxy)-5-(6-methoxy-3,4-dihydronaphthalen-1-yl)benzonitrile (4) (270 mg, yield: 53.8%) as yellow solid. ¹H NMR (400 MHz, CDCl₃) δ=7.62 (s, 1H), 7.50 (s, 1H), 6.79-6.83 (m, 2H), 6.67-6.70 (m, 1H), 5.98 (t, J=4.8 Hz, 1H), 4.49 (d, J=6.0 Hz, 2H), 3.93 (t, J=6.4 Hz, 2H), 3.83 (s, 3H), 2.82 (t, J=7.6 Hz, 2H), 2.38-2.43 (m, 2H).

3-chloro-2-(2-chloroethoxy)-5-(6-hydroxy-3,4-dihydronaphthalen-1-yl)benzonitrile (5): To a solution of 3-chloro-2-(2-chloroethoxy)-5-(6-methoxy-3,4-dihydronaphthalen-1-yl)benzonitrile (4) (270 mg, 0.72 mmol) in DCM (5 mL) was added BBr₃ (0.139 mL, 1.44 mmol) drop wise at 0° C. and the mixture was stirred at 25° C. for 4 hrs under N₂. The mixture was quenched with water (20 mL) and extracted with DCM (10 mL×3). The combined organic layers were washed with brine (30 mL), dried over Na₂SO₄, filtered and concentrated under reduced pressure. The crude was purified by silica gel column to give 3-chloro-2-(2-chloroethoxy)-5-(6-hydroxy-3,4-dihydronaphthalen-1-yl)benzonitrile (5) (130 mg, yield: 45.0%) as yellow solid. ¹H NMR (400 MHz, CDCl₃) δ=7.61 (d, J=2.0 Hz, 1H), 7.49 (d, J=2.0 Hz, 1H), 6.73-6.77 (m, 2H), 6.61-6.63 (m, 1H), 5.98 (t, J=4.4 Hz, 1H), 4.78 (s, 1H) 4.49 (d, J=6.4 Hz, 2H), 3.93 (t, J=6.0 Hz, 2H), 3.83 (s, 3H), 2.80 (t, J=8.0 Hz, 2H), 2.37-2.41 (m, 2H).

3-chloro-2-(2-chloroethoxy)-5-(6-((2-chloropyrimidin-4-yl)methoxy)-3,4-dihydronaphthalen-1-yl)benzonitrile (7): To a solution of 3-chloro-2-(2-chloroethoxy)-5-(6-hydroxy-3,4-dihydronaphthalen-1-yl)benzonitrile (5) (300 mg, 0.75 mmol) and 4-(chloromethyl)-2-methylsulfanyl-pyrimidine (6) (163 mg, 0.90 mmol) in DMF (2 mL) was added Cs₂CO₃ (488 mg, 1.50 mmol) at 25° C. The mixture was stirred at the same temperature for 16 hrs. The mixture was quenched with H₂O (5 mL). The aqueous layer was extracted with EtOAc (5 mL×3). The combined organic layers were washed with brine (5 mL×4), dried over Na₂SO₄, filtered and concentrated under reduced pressure. The residue was purified by silica gel column to give 3-chloro-2-(2-chloroethoxy)-5-(6-((2-chloropyrimidin-4-yl)methoxy)-3,4-dihydronaphthalen-1-yl)benzonitrile (7) (250 mg, yield: 65.1%) as yellow oil. ¹H NMR (400 MHz, CDCl₃) δ=8.54 (d, J=4.2 Hz, 1H), 7.60 (d, J=2.0 Hz, 1H), 7.48 (d, J=2.4 Hz, 1H), 7.20 (d, J=4.8 Hz, 1H), 6.81-6.85 (m, 2H), 6.70-6.72 (m, 1H), 6.00 (t, J=4.8 Hz, 1H), 5.11 (s, 2H), 4.49 (d, J=6.0 Hz, 2H), 3.93 (t, J=6.0 Hz, 2H), 2.84 (t, J=7.6 Hz, 2H), 2.62 (s, 3H), 2.40-2.42 (m, 2H)

N-(4-(((5-(3-chloro-4-(2-chloroethoxy)-5-cyanophenyl)-7,8-dihydronaphthalen-2-yl)oxy)methyl)pyrimidin-2-yl)methanesulfonamide (A32): To a mixture of 3-chloro-2-(2-chloroethoxy)-5-(6-((2-chloropyrimidin-4-yl)methoxy)-3,4-dihydronaphthalen-1-yl) benzonitrile (7) (100 mg, 0.21 mmol), methanesulfonamide (58.6 mg, 0.62 mmol) and Cs₂CO₃ (134 mg, 0.41 mmol) in dioxane (3 mL) was added Pd₂(dba)₃ (18.8 mg, 0.02 m mol) and Xantphos (23.8 mg, 0.04 mmol) at 25° C. and the mixture was stirred at 90° C. for 16 hrs under N₂. The mixture was filtered and the filtrate was concentrated under reduced pressure. The residue was purified by p-HPLC (FA) to give N-(4-(((5-(3-chloro-4-(2-chloroethoxy)-5-cyanophenyl)-7,8-dihydronaphthalen-2-yl)oxy)methyl)pyrimidin-2-yl)methanesulfonamide (A32) (59.7 mg, yield: 50.6%) as off-white solid. ¹H NMR (400 MHz, CDCl₃) δ=8.63 (d, J=5.2 Hz, 1H), 7.60 (d, J=2.0 Hz, 1H), 7.48 (d, J=2.0 Hz, 1H), 7.28 (br s, 1H), 6.86 (d, J=2.6 Hz, 1H), 6.82 (d, J=8.6 Hz, 1H), 6.74-6.69 (m, 1H), 6.02 (t, J=4.8 Hz, 1H), 5.12 (s, 2H), 4.50 (t, J=6.0 Hz, 2H), 3.93 (t, J=6.0 Hz, 2H), 3.48 (s, 3H), 2.83 (t, J=8.0 Hz, 2H), 2.46-2.37 (m, 2H). LCMS: (220 nm): 96.28%. Exact Mass: 544.1; found 545.1/547.1.

### Example 8: Synthesis of N-(4-(((5-(3-chloro-4-(2-chloroethoxy)-5-cyanophenyl)-5,6,7,8-tetrahydronaphthalen-2-yl)oxy)methyl)pyrimidin-2-yl)methanesulfonamide (Compound A33)

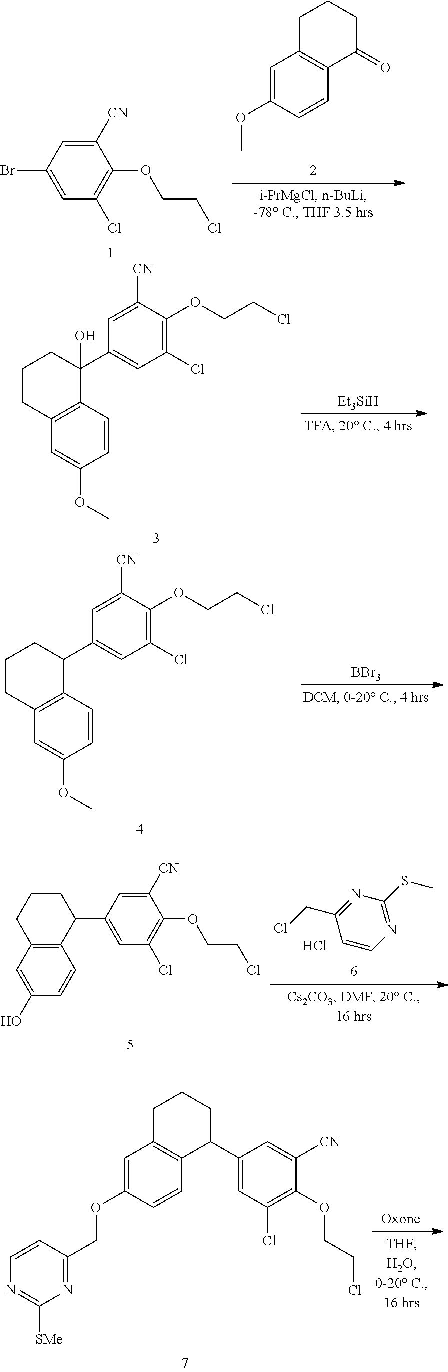

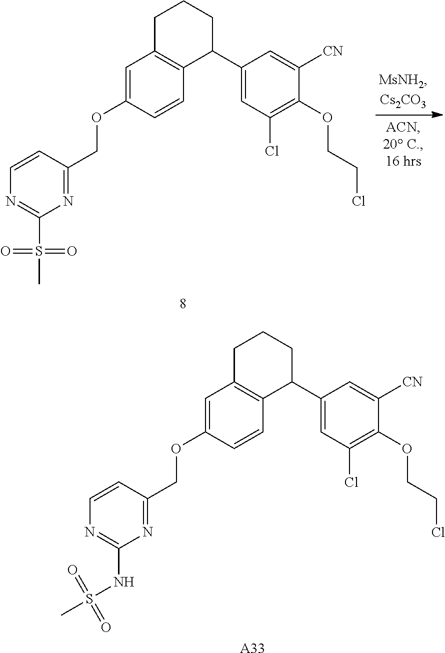 [SMILES: CS(=O)(=O)c1nccc(COc2ccc3c(c2)CCCC3c2cc(Cl)c(OCCCl)c(C#N)c2)n1 ; CS(=O)(=O)Nc1nccc(COc2ccc3c(c2)CCCC3c2cc(Cl)c(OCCCl)c(C#N)c2)n1 ; "A33"]

3-chloro-2-(2-chloroethoxy)-5-(1-hydroxy-6-methoxy-1,2,3,4-tetrahydronaphthalen-1-yl)benzonitrile (3): To a solution of n-BuLi (2500 mmol/L, 10.2 mL, 25.4 mmol) was added i-PrMgCl (2000 mmol/L, 6.36 mL, 12.7 mmol) drop wise (lots of solid appeared) at 0° C. and then to this mixture was added 5-bromo-3-chloro-2-(2-chloroethoxy)benzonitrile (1) (5 g, 0.0170 mol) in 30 mL THF at −78° C. and stirred for 30 min. Then to the mixture was added 6-methoxy-3,4-dihydronaphthalen-1(2H)-one (2) (4.48 g, 25.4 mmol) drop wise at −78° C. and stirred for 3 hrs under N₂ atmosphere. LCMS showed the start material was consumed and −30% of the product was observed. The mixture was quenched with NH₄Cl (100 mL) and extracted with EtOAc (50 mL×3). The combined organic layers were washed with brine (100 mL), dried over Na₂SO₄ and concentrated under reduced pressure to give 3-chloro-2-(2-chloroethoxy)-5-(1-hydroxy-6-methoxy-1,2,3,4-tetrahydronaphthalen-1-yl)benzonitrile (3) (8.0 g crude) as brown oil.

3-chloro-2-(2-chloroethoxy)-5-(6-methoxy-1,2,3,4-tetrahydronaphthalen-1-yl)benzonitrile (4): To a solution of 3-chloro-2-(2-chloroethoxy)-5-(1-hydroxy-6-methoxy-1,2,3,4-tetrahydronaphthalen-1-yl)benzonitrile (3) (7 g, 17.8 mmol) in TFA (70 mL) was added (C₂H5)₃SiH (8.55 mL, 53.5 mmol) at 20° C. and stirred at 20° C. for 4 hrs under N₂. LCMS showed the starting material was consumed and −30% of product was observed. The mixture was quenched with water (100 mL) and extracted with EtOAc (30 mL×3). The combined organic layers were washed with brine (100 mL), dried over Na₂SO₄, filtered and concentrated under reduced pressure. The crude was purified by silica gel column to give 3-chloro-2-(2-chloroethoxy)-5-(6-methoxy-1,2,3,4-tetrahydronaphthalen-1-yl)benzonitrile (4) (3.5 g, yield: 52.4%) as brown oil. ¹H NMR (400 MHz, CDCl₃) δ 7.63 (d, J=1.6 Hz, 1H), 7.52 (d, J=1.6 Hz, 1H), 7.35 (d, J=2.0 Hz, 1H), 7.20-7.18 (m, 2H), 4.44-4.41 (m, 2H), 4.07 (s, 1H), 3.89-3.87 (m, 2H), 2.88-2.82 (m, 2H), 2.15-2.13 (m, 1H), 1.81-1.75 (m, 3H).

3-chloro-2-(2-chloroethoxy)-5-(6-hydroxy-1,2,3,4-tetrahydronaphthalen-1-yl)benzonitrile (5): To a solution of 3-chloro-2-(2-chloroethoxy)-5-(6-methoxy-1,2,3,4-tetrahydronaphthalen-1-yl)benzonitrile (4) (3.3 g, 8.77 mmol) in DCM (30 mL) was added BBr₃ (1.69 mL, 0.0175 mol) at 0° C. and stirred at 0-20° C. for 4 hrs under N₂. LCMS showed the reaction was completed. The mixture was quenched with water (30 mL) and extracted with DCM (10 mL×3). The combined organic layers were washed with brine (30 mL), dried over Na₂SO₄, filtered and concentrated under reduced pressure. The crude was purified by silica gel column to give 3-chloro-2-(2-chloroethoxy)-5-(6-hydroxy-1,2,3,4-tetrahydronaphthalen-1-yl)benzonitrile (5) (1.5 g, yield: 47.4%) as brown oil. ¹H NMR (400 MHz, CDCl₃) δ=7.35 (d, J=2.0 Hz, 1H), 7.19 (d, J=2.0 Hz, 1H), 6.65-6.59 (m, 3H), 4.94 (s, 1H), 4.42 (t, J=6.0 Hz, 2H), 4.04 (t, J=6.8 Hz, 1H), 3.88 (t, J=6.0 Hz, 2H), 2.85-2.78 (m, 2H), 2.13-2.12 (m, 1H), 1.78-1.74 (m, 3H).

3-chloro-2-(2-chloroethoxy)-5-(6-((2-(methylthio)pyrimidin-4-yl)methoxy)-1,2,3,4-tetrahydronaphthalen-1-yl)benzonitrile (7): To a solution of 3-chloro-2-(2-chloroethoxy)-5-(6-hydroxy-1,2,3,4-tetrahydronaphthalen-1-yl)benzonitrile (5) (0.5 g, 1.39 mmol) in DMF (5 mL) was added 4-(chloromethyl)-2-methylsulfanyl-pyrimidine (6) (0.35 g, 1.67 mmol), Cs₂CO₃ (1.13 g, 3.47 mmol) at 20° C. and stirred at 20° C. for 16 hrs under N₂. LCMS showed the reaction was completed. The mixture was quenched with water (20 mL) and extracted with EtOAc (10 mL×3). The combined organic layers were washed with brine (30 mL×4), dried over Na₂SO₄, filtered and concentrated under reduced pressure to give 3-chloro-2-(2-chloroethoxy)-5-(6-((2-(methylthio)pyrimidin-4-yl)methoxy)-1,2,3,4-tetrahydronaphthalen-1-yl)benzonitrile (7) (0.5 g, yield: 72.1%) as brown oil. ¹H NMR (400 MHz, CDCl₃) δ 8.54 (d, J=5.2 Hz, 1H), 7.35 (d, J=2.0 Hz, 1H), 7.21 (d, J=5.2 Hz, 1H), 7.18 (d, J=2.0 Hz, 1H), 6.74 (s, 1H), 6.70 (s, 2H), 5.08 (s, 2H), 4.43 (t, J=6.0 Hz, 2H), 4.06 (t, J=2.0 Hz, 1H), 3.88 (t, J=6.0 Hz, 2H), 2.89-2.80 (m, 2H), 2.58 (s, 3H), 2.14-2.13 (m, 1H), 1.79-1.75 (m, 3H).

3-chloro-2-(2-chloroethoxy)-5-(6-((2-(methylsulfonyl)pyrimidin-4-yl)methoxy)-1,2,3,4-tetrahydronaphthalen-1-yl)benzonitrile (8): To a solution of 3-chloro-2-(2-chloroethoxy)-5-(6-((2-(methylthio)pyrimidin-4-yl)methoxy)-1,2,3,4-tetrahydronaphthalen-1-yl)benzonitrile (7) (0.330 g, 0.659 mmol) (6) in THF/H₂O (6 mL, v:v=1/1) was added Oxone (0.921 g, 1.5 mmol) at 0° C. and stirred at 20° C. for 16 hrs under N₂. LCMS showed the reaction was completed. The mixture was quenched with water (10 mL) and extracted with EtOAc (5 mL×3). The combined organic layers were washed with brine (20 mL), dried over Na₂SO₄, filtered and concentrated under reduced pressure to give 3-chloro-2-(2-chloroethoxy)-5-(6-((2-(methylsulfonyl)pyrimidin-4-yl)methoxy)-1,2,3,4-tetrahydronaphthalen-1-yl)benzonitrile (8) (330 mg, yield: 93.1%) as brown oil. ¹H NMR (400 MHz, CDCl₃) δ 8.95 (d, J=5.2 Hz, 1H), 7.85 (d, J=5.2 Hz, 1H), 7.36 (d, J=2.0 Hz, 1H), 7.17 (d, J=2.0 Hz, 1H), 6.75 (s, 1H), 6.73 (s, 2H), 5.29 (s, 2H), 4.43 (t, J=6.0 Hz, 2H), 4.08 (br t, J=6.4 Hz, 1H), 3.89 (t, J=6.0 Hz, 2H), 3.40 (s, 3H), 2.95-2.73 (m, 1H), 2.21-2.09 (m, 1H), 1.86-1.71 (m, 3H).

N-(4-(((5-(3-chloro-4-(2-chloroethoxy)-5-cyanophenyl)-5,6,7,8-tetrahydronaphthalen-2-yl)oxy)methyl)pyrimidin-2-yl)methanesulfonamide (A33): To a solution of 3-chloro-2-(2-chloroethoxy)-5-(6-((2-(methylsulfonyl)pyrimidin-4-yl)methoxy)-1,2,3,4-tetrahydronaphthalen-1-yl)benzonitrile (8) (0.3 g, 0.563 mmol) in MeCN (3 mL) was added Methanesulfonamide (0.161 g, 1.69 mmol) and Cs₂CO₃ (0.551 g, 1.69 mmol) at 20° C. and stirred at 20° C. for 16 hrs under N₂. LCMS showed the reaction was completed. The mixture was quenched with water (10 mL) and adjusted pH=3 with 3N HCl. The mixture was filtered and the filter cake was washed with CH₃CN (5 mL) and dried under reduced pressure to give N-(4-(((5-(3-chloro-4-(2-chloroethoxy)-5-cyanophenyl)-5,6,7,8-tetrahydronaphthalen-2-yl)oxy)methyl)pyrimidin-2-yl)methanesulfonamide (A33) (80 mg, yield: 24.6%) as white solid. ¹H NMR (400 MHz, CDCl₃) δ 8.64 (d, J=5.2 Hz, 1H), 7.35 (d, J=2.0 Hz, 1H), 7.29 (d, J=5.2 Hz, 1H), 7.17 (d, J=2.0 Hz, 1H), 6.76 (s, 1H), 6.72 (s, 2H), 5.10 (s, 2H), 4.43 (t, J=6.0 Hz, 2H), 4.07 (t, J=5.2 Hz, 1H), 3.89 (t, J=6.0 Hz, 2H), 3.48 (s, 3H), 2.96-2.75 (m, 1H), 2.21-2.09 (m, 1H), 1.85-1.72 (m, 3H). LCMS: (220 nm): 94.47%. Exact Mass: 546.1; found 547.0/549.0.

### Example 9: Synthesis of N-(4-(((5-(3-chloro-4-(2-chloroethoxy)-5-cyanophenyl)naphthalen-2-yl)oxy)methyl)pyrimidin-2-yl)methanesulfonamide (Compound A34)

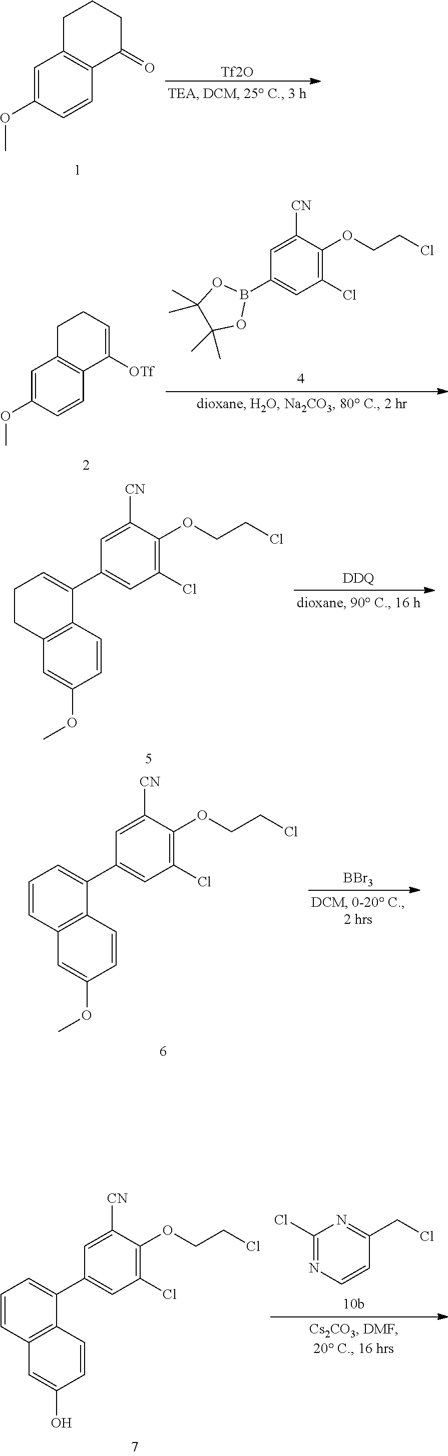

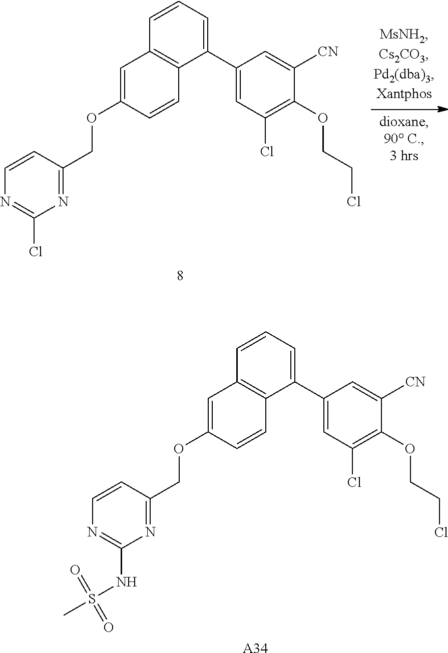 [SMILES: N#Cc1cc(-c2cccc3cc(OCc4ccnc(Cl)n4)ccc23)cc(Cl)c1OCCCl ; CS(=O)(=O)Nc1nccc(COc2ccc3c(-c4cc(Cl)c(OCCCl)c(C#N)c4)cccc3c2)n1 ; "A34"]

6-methoxy-3,4-dihydronaphthalen-1-yl trifluoromethanesulfonate (2): To a solution of 6-methoxytetralin-1-one (1) (5.00 g, 25.5 mmol) and TEA (10.7 mL, 76.6 mmol) in DCM (50 mL) was added Trifluoromethanesulfonic anhydride (7.77 mL, 46.0 mmol) at 0° C. and stirred at 25° C. for 3 hrs. TLC showed the starting material remained and a new spot appeared. The mixture was added to water (50 mL) and extracted with DCM (50 mL×3). The combined organic layers were washed with brine (50 mL), dried over Na₂SO₄, filtered and concentrated under reduced pressure. The crude product was purified by silica gel column to give (6-methoxy-3,4-dihydronaphthalen-1-yl)trifluoromethanesulfonate (2) (6.30 g, yield: 68.0%) as yellow solid. ¹H NMR (400 MHz, CDCl₃) δ=7.27 (s, 1H), 7.25 (s, 1H), 6.78-6.70 (m, 2H), 5.85 (t, J=4.8 Hz, 1H), 3.80 (s, 3H), 2.82 (t, J=8.2 Hz, 2H), 2.47 (dt, J=4.8, 8.1 Hz, 2H).

3-chloro-2-(2-chloroethoxy)-5-(6-methoxy-3,4-dihydronaphthalen-1-yl)benzonitrile (5): To a solution of (6-methoxy-3,4-dihydronaphthalen-1-yl)trifluoromethanesulfonate (2) (2.0 g) in 1,4-dioxane (20 mL) and H₂O (4 mL) was added 3-chloro-2-(2-chloroethoxy)-5-(4,4,5,5-tetramethyl-1,3,2-dioxaborolan-2-yl)benzonitrile (4) (2.0 g), Na₂CO₃ (1.24 g, 0.0117 mol) and Tetrakis(triphenylphosphine)palladium(0) (0.676 g, 0.000585 mol) at 20° C. The reaction was stirred at 80° C. for 2 hrs under N₂. The mixture was quenched with water (40 mL) and extracted with EtOAc (20 mL×3). The combined organic layers were washed with brine (40 mL), dried over Na₂SO₄, filtered and concentrated under reduced pressure. The crude product was purified by column to give 3-chloro-2-(2-chloroethoxy)-5-(6-methoxy-3,4-dihydronaphthalen-1-yl)benzonitrile (5) (0.660 g, yield: 27.1%) as white solid. ¹H NMR (400 MHz, CDCl₃) δ=7.62 (d, J=2.0 Hz, 1H), 7.50 (d, J=2.2 Hz, 1H), 6.85-6.76 (m, 2H), 6.68 (dd, J=2.6, 8.4 Hz, 1H), 5.98 (t, J=4.8 Hz, 1H), 4.49 (t, J=6.2 Hz, 2H), 3.93 (t, J=6.2 Hz, 2H), 3.83 (s, 3H), 2.82 (t, J=7.8 Hz, 2H), 2.40 (dt, J=4.8, 7.8 Hz, 2H).

3-chloro-2-(2-chloroethoxy)-5-(6-methoxynaphthalen-1-yl)benzonitrile (6): To a solution of 3-chloro-2-(2-chloroethoxy)-5-(6-methoxy-3,4-dihydronaphthalen-1-yl) benzonitrile (5) (1.80 g) in dioxane (20 mL) was added DDQ (1.64 g, 07.22 mmol) at 20° C. and stirred at 90° C. for 16 hrs under N₂. The mixture was filtered and concentrated under reduced pressure to give a residue. The residue was purified by silica gel column to give 3-chloro-2-(2-chloroethoxy)-5-(6-methoxynaphthalen-1-yl) benzonitrile (6) (1.23 g, yield: 61.7%) as white solid. ¹H NMR (400 MHz, CDCl₃) δ=7.82 (d, J=8.4 Hz, 1H), 7.75 (d, J=2.2 Hz, 1H), 7.66-7.61 (m, 2H), 7.49 (dd, J=7.2, 8.2 Hz, 1H), 7.24-7.20 (m, 2H), 7.18 (dd, J=2.6, 9.3 Hz, 1H), 4.56 (t, J=6.2 Hz, 2H), 3.98-3.95 (m, 5H).

3-chloro-2-(2-chloroethoxy)-5-(6-hydroxynaphthalen-1-yl)benzonitrile (7): To a solution of 3-chloro-2-(2-chloroethoxy)-5-(6-methoxynaphthalen-1-yl)benzonitrile (6) (1.23 g) in DCM (15 mL) was added BBr₃ (0.637 mL, 6.61 mmol) at 0° C. and stirred at 0-20° C. for 2 hrs under N₂. The mixture was quenched with water (20 mL) and extracted with DCM (15 mL×3). The combined organic layers were washed with brine (30 mL), dried over Na₂SO₄, filtered and concentrated under reduced pressure. The crude was purified by silica gel column to give 3-chloro-2-(2-chloroethoxy)-5-(6-hydroxy-1-naphthyl)benzonitrile (7) (0.60 g, yield: 45.6%) as yellow oil. ¹H NMR (400 MHz, CDCl₃) δ=7.78-7.73 (m, 2H), 7.67-7.61 (m, 2H), 7.48 (dd, J=7.2, 8.3 Hz, 1H), 7.26 (d, J=2.6 Hz, 1H), 7.21 (dd, J=1.2, 7.1 Hz, 1H), 7.13 (dd, J=2.6, 9.1 Hz, 1H), 5.25 (s, 1H), 4.56 (t, J=6.2 Hz, 2H), 3.96 (t, J=6.2 Hz, 2H).

3-chloro-2-(2-chloroethoxy)-5-(6-((2-chloropyrimidin-4-yl)methoxy)naphthalen-1-yl)benzonitrile (8): To a solution of 3-chloro-2-(2-chloroethoxy)-5-(6-hydroxy-1-naphthyl)benzonitrile (7) (0.300 g, 0.840 mmol) in DMF (6 mL) was added 2-chloro-4-(chloromethyl)pyrimidine (0.164 g, 0.904 mmol) and Cs₂CO₃ (0.491 g, 0.151 mmol) at 20° C. The mixture was stirred at the same temperature for 16 hrs. LCMS showed the reaction was completed and the resulting mixture was quenched with H₂O (10 mL). The aqueous layer was extracted with EtOAc (10 mL×3). The combined organic layers were washed with brine (5 mL×4), dried over Na₂SO₄, filtered and concentrated under reduced pressure to give a crude product. The crude product was purified by silica gel column to give 3-chloro-2-(2-chloroethoxy)-5-(6-((2-chloropyrimidin-4-yl)methoxy)naphthalen-1-yl)benzonitrile (8) (0.110 g, yield: 28.6%) as yellow oil. ¹H NMR (400 MHz, CDCl₃) δ=8.67 (d, J=5.0 Hz, 1H), 7.80 (d, J=8.4 Hz, 1H), 7.75 (d, J=2.2 Hz, 1H), 7.71 (d, J=9.4 Hz, 1H), 7.63 (d, J=2.2 Hz, 1H), 7.59 (d, J=5.0 Hz, 1H), 7.55-7.49 (m, 1H), 7.30-7.27 (m, 1H), 7.26 (s, 1H), 7.23 (d, J=2.6 Hz, 1H), 5.32 (s, 2H), 4.57 (t, J=6.2 Hz, 2H), 3.97 (t, J=6.2 Hz, 2H).

N-(4-(((5-(3-chloro-4-(2-chloroethoxy)-5-cyanophenyl)naphthalen-2-yl)oxy)methyl)pyrimidin-2-yl)methanesulfonamide (A34): To a mixture of 3-chloro-2-(2-chloroethoxy)-5-(6-((2-chloropyrimidin-4-yl)methoxy)naphthalen-1-yl)benzonitrile (8) (0.200 g, 0.413 mmol), methanesulfonamide (0.118 g, 1.24 mmol), Cs₂CO₃ (0.269 g, 0.825 mmol) and Xantphos (0.0477 g, 0.0825 mmol) in dioxane (6 mL) was added Pd₂(dba)₃ (0.0378 g, 0.0413 mmol) and stirred at 90° C. for 16 hrs under N₂ atmosphere. The mixture was poured into water (10 mL) and extracted with EtOAc (10 mL×3), the organic layers were washed with brine (20 mL), dried over Na₂SO₄, filtered and concentrated under reduced pressure to give a crude product. The crude product was purified by prep-HPLC (neutral) to give N-(4-(((5-(3-chloro-4-(2-chloroethoxy)-5-cyanophenyl)naphthalen-2-yl)oxy)methyl)pyrimidin-2-yl)methanesulfonamide (A34) (0.0367 g, yield: 16.4%) as white solid. ¹H NMR (400 MHz, DMSO-d₆) δ=8.61 (d, J=5.2 Hz, 1H), 7.99-7.88 (m, 3H), 7.72 (d, J=9.4 Hz, 1H), 7.60-7.52 (m, 2H), 7.37-7.32 (m, 2H), 7.20 (br s, 1H), 5.28 (s, 2H), 4.61-4.51 (m, 2H), 4.09-4.01 (m, 2H), 3.31 (br s, 3H). LCMS: (220 nm): 96.57%, Exact Mass: 542.1; found 543.0/545.0.

### Example 10: Synthesis of N-(4-(((1-(3-chloro-4-(2-chloroethoxy)-5-cyanophenyl)-2,3-dihydro-1H-inden-5-yl)oxy)methyl)pyrimidin-2-yl)methanesulfonamide (Compound A35)

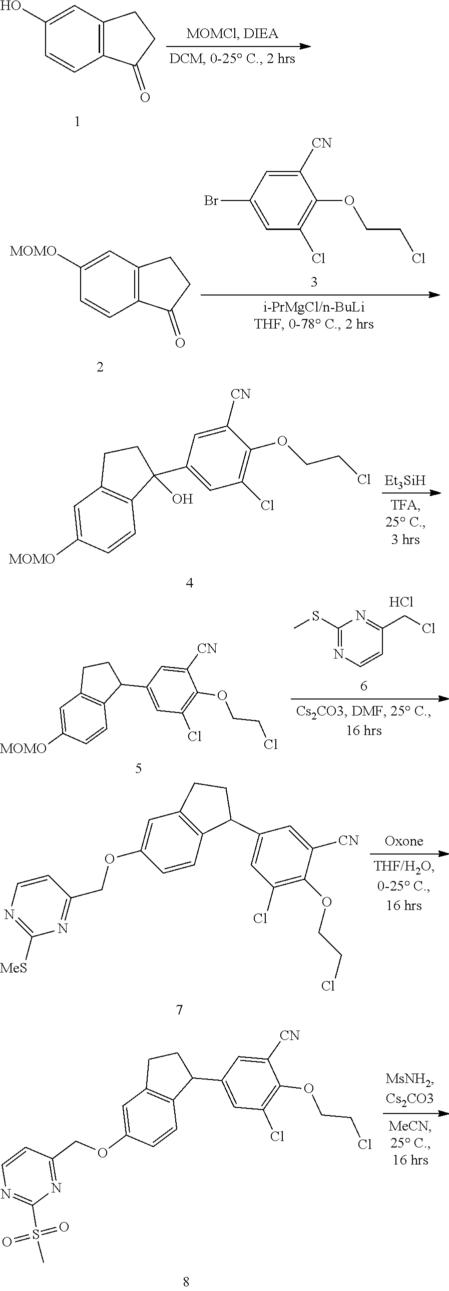

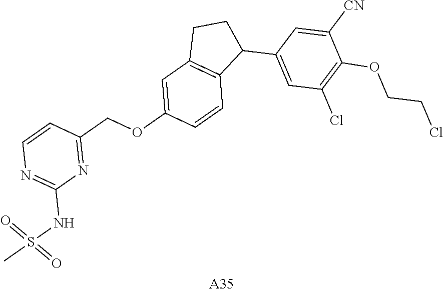 [SMILES: CS(=O)(=O)Nc1nccc(COc2ccc3c(c2)CCC3c2cc(Cl)c(OCCCl)c(C#N)c2)n1 ; "A35"]

5-(methoxymethoxy)-2,3-dihydro-1H-inden-1-one (2): To a solution of 5-hydroxy-2,3-dihydro-1H-inden-1-one (1) (3.00 g, 20.2 mmol) and DIEA (10.4 mL, 60.7 mmol) in DCM (50 mL) was added chloro(methoxy)methane (3.26 g, 40.5 mmol) at 0° C. and the mixture was stirred at 25° C. for 2 hrs. The mixture was quenched with water (50 mL) and extracted with DCM (20 mL×3). The combined organic layers were washed with brine (50 mL), dried over Na₂SO₄, filtered and concentrated under reduced pressure. The residue was purified by silica gel column to give 5-(methoxymethoxy)-2,3-dihydro-1H-inden-1-one (2) (3.10 g, yield: 71.7%) as light oil. ¹H NMR (400 MHz, CDCl₃) δ=7.70 (d, J=8.4 Hz, 1H), 7.09 (d, J=1.6 Hz, 1H), 7.06-6.97 (m, 1H), 5.26 (s, 2H), 3.50 (s, 3H), 3.13-3.07 (m, 2H), 2.71-2.65 (m, 2H).

3-chloro-2-(2-chloroethoxy)-5-(1-hydroxy-5-(methoxymethoxy)-2,3-dihydro-1H-inden-1-yl)benzonitrile (4): To n-BuLi (2500 mmol/L, 8.14 mL, 0.0203 mol) was added i-PrMgCl (2000 mmol/L, 5.09 mL, 0.0102 mol) drop wise at 0° C. and the mixture was stirred at 0° C. for 30 min. Then to this mixture was added 5-bromo-3-chloro-2-(2-chloroethoxy)benzonitrile (3) (4.00 g, 13.6 mmol) in THF (20 mL) at −78° C. and stirred at −78° C. for 30 min under N₂. Then to this mixture was added 5-(methoxymethoxy)indan-1-one (3.13 g, 16.3 mmol) in THF (30 mL) drop wise at −78° C. and stirred for 2 hrs under N₂. LCMS showed the reaction was completed. The mixture was poured into sat. NH₄Cl (100 mL) and extracted with EtOAc (50 mL×3), the combined organic layers were washed with brine (50 mL×2), dried over Na₂SO₄, filtered and concentrated under reduced pressure. The residue was purified by p-HPLC (FA) to give 3-chloro-2-(2-chloroethoxy)-5-(1-hydroxy-5-(methoxymethoxy)-2,3-dihydro-1H-inden-1-yl) benzonitrile (4) (0.310 g, yield: 5.04%) as a light oil. ¹H NMR (400 MHz, CDCl₃) δ=7.84 (d, J=2.0 Hz, 1H), 7.72 (d, J=2.0 Hz, 1H), 7.37 (d, J=8.4 Hz, 1H), 7.29 (d, J=2.0 Hz, 1H), 7.07-7.04 (m, 1H), 6.53 (t, J=2.0 Hz, 1H), 5.23 (s, 2H), 4.51 (t, J=6.2 Hz, 2H), 4.45 (t, J=6.2 Hz, 1H), 3.94 (t, J=6.2 Hz, 2H), 3.52 (s, 3H), 3.24-3.11 (m, 1H), 3.02-2.87 (m, 1H), 2.54-2.37 (m, 1H).

3-chloro-2-(2-chloroethoxy)-5-(5-(methoxymethoxy)-2,3-dihydro-1H-inden-1-yl) benzonitrile (5): To a solution of 3-chloro-2-(2-chloroethoxy)-5-(1-hydroxy-5-(methoxymethoxy)-2,3-dihydro-1H-inden-1-yl)benzonitrile (4) (0.310 g, 0.76 mmol) in TFA (3 mL) was added (C₂H5)₃SiH (0.364 mL, 2.28 mmol) at 25° C. for 3 hrs. The mixture was poured into water (10 mL) and extracted with DCM (5 mL×3). The combined organic layers were washed with brine (5 mL×3), dried over Na₂SO₄, filtered and concentrated under reduced pressure. The residue was purified by p-TLC to give 3-chloro-2-(2-chloroethoxy)-5-(5-(methoxymethoxy)-2,3-dihydro-1H-inden-1-yl)benzonitrile (5) (0.160 g, yield: 54.5%) as a yellow oil. ¹H NMR (400 MHz, CDCl₃) δ=7.42 (d, J=2.0 Hz, 1H), 7.29 (d, J=2.0 Hz, 1H), 6.81-6.76 (m, 2H), 6.69-6.63 (m, 1H), 4.43 (t, J=6.2 Hz, 2H), 4.26 (t, J=8.0 Hz, 1H), 3.89 (t, J=6.2 Hz, 2H), 3.04-2.86 (m, 2H), 2.65-2.56 (m, 1H), 2.01-1.90 (m, 1H).

3-chloro-2-(2-chloroethoxy)-5-(5-((2-(methylthio)pyrimidin-4-yl)methoxy)-2,3-dihydro-1H-inden-1-yl)benzonitrile (7): To a solution of 3-chloro-2-(2-chloroethoxy)-5-(5-(methoxymethoxy)-2,3-dihydro-1H-inden-1-yl)benzonitrile (5) (160 mg, 0.46 mmol) and Cs₂CO₃ (0.524 g, 1.61 mmol) in DMF (2 mL) was added 4-(chloromethyl)-2-methylsulfanyl-pyrimidine (6) (145 mg, 0.69 mmol) and stirred at 25° C. for 16 hrs. The mixture was poured into water (5 mL) and extracted with EtOAc (5 mL×3), the combined organic layers were washed with brine (5 mL×4), dried over Na₂SO₄, filtered and concentrated under reduced pressure to give a residue. The residue was purified by silica gel column to give 3-chloro-2-(2-chloroethoxy)-5-(5-((2-(methylthio)pyrimidin-4-yl)methoxy)-2,3-dihydro-1H-inden-1-yl) benzonitrile (7) (160 mg, yield: 64.4%) as a yellow solid. ¹H NMR (400 MHz, CDCl₃) δ=8.55 (d, J=5.0 Hz, 1H), 7.42 (d, J=2.0 Hz, 1H), 7.29 (d, J=2.2 Hz, 1H), 7.23 (d, J=5.2 Hz, 1H), 6.90 (d, J=1.6 Hz, 1H), 6.87-6.83 (m, 1H), 6.81-6.77 (m, 1H), 5.11 (s, 2H), 4.44 (t, J=6.0 Hz, 2H), 4.27 (t, J=8.0 Hz, 1H), 3.89 (t, J=6.2 Hz, 2H), 3.06-2.91 (m, 2H), 2.65-2.56 (m, 4H), 2.03-1.91 (m, 1H).

3-chloro-2-(2-chloroethoxy)-5-(5-((2-(methylsulfonyl)pyrimidin-4-yl)methoxy)-2,3-dihydro-1H-inden-1-yl)benzonitrile (8): To a solution of 3-chloro-2-(2-chloroethoxy)-5-(5-((2-(methylthio)pyrimidin-4-yl)methoxy)-2,3-dihydro-1H-inden-1-yl)benzonitrile (7) (160 mg, 0.33 mmol) in THF (2 mL) and water (2 mL) was added Oxone (506 mg, 0.82 mmol) at 25° C. and the mixture was stirred at 25° C. for 16 hrs. The mixture was poured into water (5 mL) and extracted with EtOAc (5 mL×3), the combined organic layers were washed with brine (5 mL×2), dried over Na₂SO₄, filtered and concentrated under reduce pressure to give 3-chloro-2-(2-chloroethoxy)-5-(5-((2-(methylsulfonyl)pyrimidin-4-yl)methoxy)-2,3-dihydro-1H-inden-1-yl) benzonitrile (8) (120 mg, yield: 63.3%) as yellow solid. ¹H NMR (400 MHz, CDCl₃) δ=8.94 (d, J=5.0 Hz, 1H), 7.85 (d, J=5.0 Hz, 1H), 7.43 (d, J=2.2 Hz, 1H), 7.28 (d, J=2.2 Hz, 1H), 6.91 (d, J=1.6 Hz, 1H), 6.89-6.86 (m, 1H), 6.82-6.78 (m, 1H), 5.31 (s, 2H), 4.44 (t, J=6.0 Hz, 2H), 4.29 (t, J=7.8 Hz, 1H), 3.90 (t, J=6.2 Hz, 2H), 3.41 (s, 3H), 3.06-2.94 (m, 2H), 2.68-2.59 (m, 1H), 2.02-1.95 (m, 1H).

N-(4-(((1-(3-chloro-4-(2-chloroethoxy)-5-cyanophenyl)-2,3-dihydro-1H-inden-5-yl)oxy)methyl)pyrimidin-2-yl)methanesulfonamide (A33): To a solution of 3-chloro-2-(2-chloroethoxy)-5-(5-((2-(methylsulfonyl)pyrimidin-4-yl)methoxy)-2,3-dihydro-1H-inden-1-yl)benzonitrile (8) (120 mg, 0.23 mmol) and MsNH₂ (55 mg, 0.58 mmol) in MeCN (3 mL) was added Cs₂CO₃ (189 mg, 0.58 mmol) at 25° C. and the mixture was stirred at 25° C. for 16 hrs. The mixture was added 3N HCl and adjust pH=6, then the mixture was poured into water (20 mL) and extracted with EtOAc (10 mL×3), the combined organic were washed with brine (10 mL×4), dried over Na₂SO₄, filtered and concentrated under reduced pressure to give a residue. The residue was purified by p-HPLC (FA) to give N-(4-(((1-(3-chloro-4-(2-chloroethoxy)-5-cyanophenyl)-2,3-dihydro-1H-inden-5-yl)oxy)methyl)pyrimidin-2-yl)methanesulfonamide (A33) (48 mg, yield: 35.0%) as a white solid. ¹H NMR (400 MHz, CDCl₃) δ=8.63 (d, J=5.2 Hz, 1H), 7.43 (d, J=2.2 Hz, 1H), 7.32-7.28 (m, 2H), 6.91 (s, 1H), 6.88-6.84 (m, 1H), 6.82-6.77 (m, 1H), 5.12 (s, 2H), 4.44 (t, J=6.2 Hz, 2H), 4.28 (t, J=7.8 Hz, 1H), 3.89 (t, J=6.2 Hz, 2H), 3.49 (s, 3H), 3.06-2.91 (m, 2H), 2.69-2.58 (m, 1H), 2.03-1.94 (m, 1H). LCMS: (220 nm): 99.58%. Exact Mass: 532.1; found 533.0/535.0.

### Example 11: Synthesis of N-(4-(((1-(3-chloro-4-(2-chloroethoxy)-5-cyanophenyl)-1H-indol-5-yl)oxy)methyl)pyrimidin-2-yl)methanesulfonamide (Compound A36)

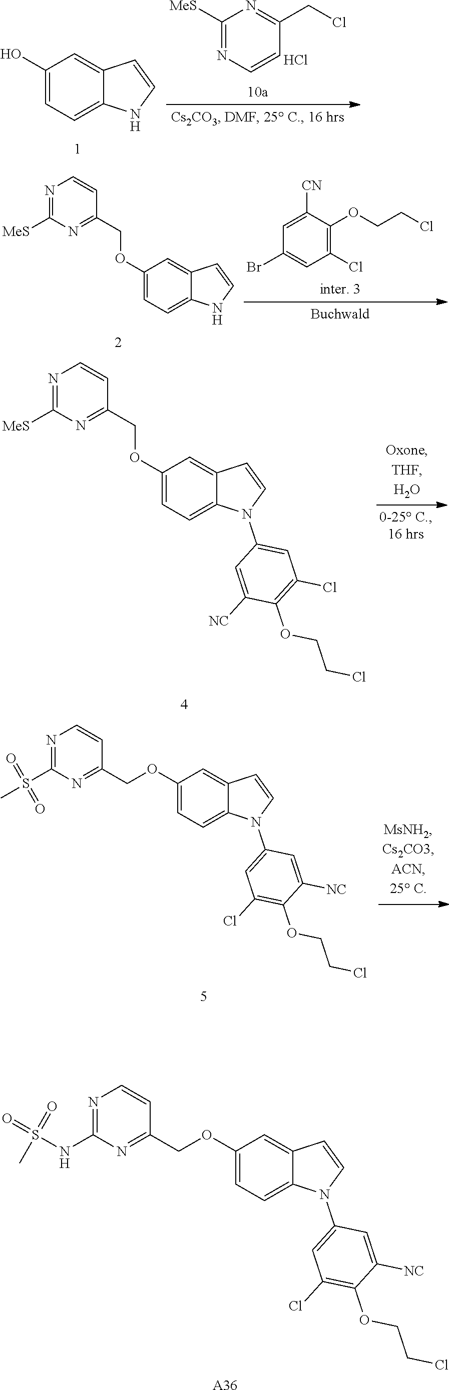

5-((2-(methylthio)pyrimidin-4-yl)methoxy)-1H-indole (2): To a solution of 1H-indol-5-ol (3 g, 0.0225 mol) in DMF (50 mL) was added 4-(chloromethyl)-2-(methylthio)pyrimidine (5.23 g, 0.0248 mol) and Cs₂CO₃ (18.4 g, 0.0563 mol) at 20° C. and the mixture was stirred at 20° C. for 16 hrs under N₂. LCMS showed the starting material was consumed. The solution was poured into water (100 mL), extracted with EtOAc (50 mL×3). The combine organic layers were washed with brine (100 mL×4), dried over Na₂SO₄, filtered and concentrated under reduced pressure. The residue was purified by silica gel column to give 5-((2-(methylthio)pyrimidin-4-yl)methoxy)-1H-indole (5 g, yield: 82.00o) as yellow oil. ¹HNMR (400 MHz, CDCl₃) δ=10.95 (s, 1H), 8.63 (d, J=5.2 Hz, 1H), 7.32-7.28 (m, 3H), 7.10 (d, J=2.4 Hz, 1H), 6.86-6.83 (m, 1H), 5.13 (s, 2H), 2.59 (s, 3H).

3-chloro-2-(2-chloroethoxy)-5-(5-((2-(methylthio)pyrimidin-4-yl)methoxy)-1H-indol-1-yl)benzonitrile (3): To a solution of 5-((2-(methylthio)pyrimidin-4-yl)methoxy)-1H-indole (0.506 g, 1.86 mmol) in toluene (5 mL) was added 5-bromo-3-chloro-2-(2-chloroethoxy)benzonitrile (0.5 g, 1.70 mmol), Pd(OAc)₂ (76.1 mg, 0.339 mmol), K₃PO₄ (0.720 g, 3.39 mmol), and X-Phos (0.147 g, 0.339 mmol) at 20° C. and stirred at 60° C. for 16 hrs under N₂. The mixture was quenched with water (10 mL) and extracted with EtOAc (10 mL×3). The combined organic layers were washed with brine (10 mL), dried over Na₂SO₄, filtered and concentrated under reduced pressure. The crude was purified by silica gel column to give 3-chloro-2-(2-chloroethoxy)-5-(5-((2-(methylthio)pyrimidin-4-yl)methoxy)-1H-indol-1-yl)benzonitrile (0.150 g, 0.247 mmol) as brown oil. ¹H NMR (400 MHz, CDCl₃) δ=8.53 (d, J=5.2 Hz, 1H), 7.78 (d, J 2.8 Hz, 1H), 7.64 (d, J=2.4 Hz, 1H), 7.41 (d, J=8.8 Hz, 1H), 7.25 (d, J=6.4 Hz, 2H), 7.23 (s, 1H), 7.13 (d, J 2.4 Hz, 1H), 6.65 (d, J=3.2 Hz, 1H), 5.16 (s, 2H), 4.53 (t, J=5.2 Hz, 2H), 3.94 (t, J=6.0 Hz, 2H), 2.61 (s, 4H).

3-chloro-2-(2-chloroethoxy)-5-(5-((2-(methylsulfonyl)pyrimidin-4-yl)methoxy)-1H-indol-1-yl)benzonitrile (4): To a solution of 3-chloro-2-(2-chloroethoxy)-5-(5-((2-(methylthio)pyrimidin-4-yl)methoxy)-1H-indol-1-yl)benzonitrile (0.15 g, 0.309 mmol) (3) in THF (1.5 mL)/H₂O (1.5 mL) was added Oxone (0.475 g, 0.773 mmol) at 20° C. and the mixture was stirred at 20° C. for 16 hrs under N₂. The mixture was quenched with water (10 mL) and extracted with EtOAc (10 mL×3). The combined organic layers were washed with brine (10 mL), dried over Na₂SO₄, filtered and concentrated under reduced pressure. The crude was purified by p-TLC to give 3-chloro-2-(2-chloroethoxy)-5-(5-((2-(methylsulfonyl)pyrimidin-4-yl)methoxy)-1H-indol-1-yl)benzonitrile (0.11 g, yield: 73.6%) as brown oil. ¹H NMR (400 MHz, CDCl₃) δ=8.92 (d, J=5.2 Hz, 1H), 7.88 (d, J=5.2 Hz, 1H), 7.78 (d, J=2.4 Hz, 1H), 7.64 (d, J=2.4 Hz, 1H), 7.43 (d, J=8.8 Hz, 1H), 7.25 (s, 1H), 7.15 (d, J=2.0 Hz, 1H), 7.04 (d, J=2.4 Hz, 1H), 6.67 (d, J=3.2 Hz, 1H), 4.54 (t, J=10.0 Hz, 2H), 3.94 (t, J=6.0 Hz, 2H), 3.41 (s, 3H).

N-(4-(((1-(3-chloro-4-(2-chloroethoxy)-5-cyanophenyl)-1H-indol-5-yl)oxy)methyl)pyrimidin-2-yl)methanesulfonamide (A36): To a solution of 3-chloro-2-(2-chloroethoxy)-5-(5-((2-(methylsulfonyl)pyrimidin-4-yl)methoxy)-1H-indol-1-yl)benzonitrile (60 mg, 0.116 mmol) (4) in MeCN (1 mL) was added methanesulfonamide (33.1 mg, 0.348 mmol) and Cs₂CO₃ (0.113 g, 0.348 mmol) at 20° C. The mixture was stirred at the same temperature for 16 hrs. LCMS showed the reaction was completed. The mixture was quenched with water (10 mL) and extracted with EtOAc (5 mL×3). The combined organic layers were washed with brine (20 mL), dried over Na₂SO₄, filtered and concentrated under reduced pressure. The crude was purified by p-HPLC(FA) to give N-(4-(((1-(3-chloro-4-(2-chloroethoxy)-5-cyanophenyl)-1H-indol-5-yl)oxy)methyl)pyrimidin-2-yl)methanesulfonamide (0.0101 g, yield: 16.2%) as white solid. ¹H NMR (400 MHz, CDCl₃) δ=8.63 (s, 1H), 8.62 (s, 1H), 7.78 (d, J=2.4 Hz, 1H), 7.64 (d, J=2.8 Hz, 1H), 7.33 (d, J=5.2 Hz, 1H), 7.32 (s, 1H), 7.25 (d, J=3.2 Hz, 1H), 7.16 (d, J=2.4 Hz, 1H), 7.03 (d, J=2.4 Hz, 1H), 6.66 (d, J=3.6 Hz, 1H) 5.18 (s, 2H), 4.53 (t, J=6.0 Hz, 2H), 3.94 (t, J=6.0 Hz, 2H), 3.49 (s, 3H). LCMS: (220 nm): 99.46%. Exact Mass: 531.1; found 532.1/534.1.

### Example 12: Synthesis of N-(4-(((5-(3-chloro-4-(2-chloroethoxy)-5-cyanophenyl)-1,3,4-oxadiazol-2-yl)oxy)methyl)pyrimidin-2-yl)methanesulfonamide (Compound A37)

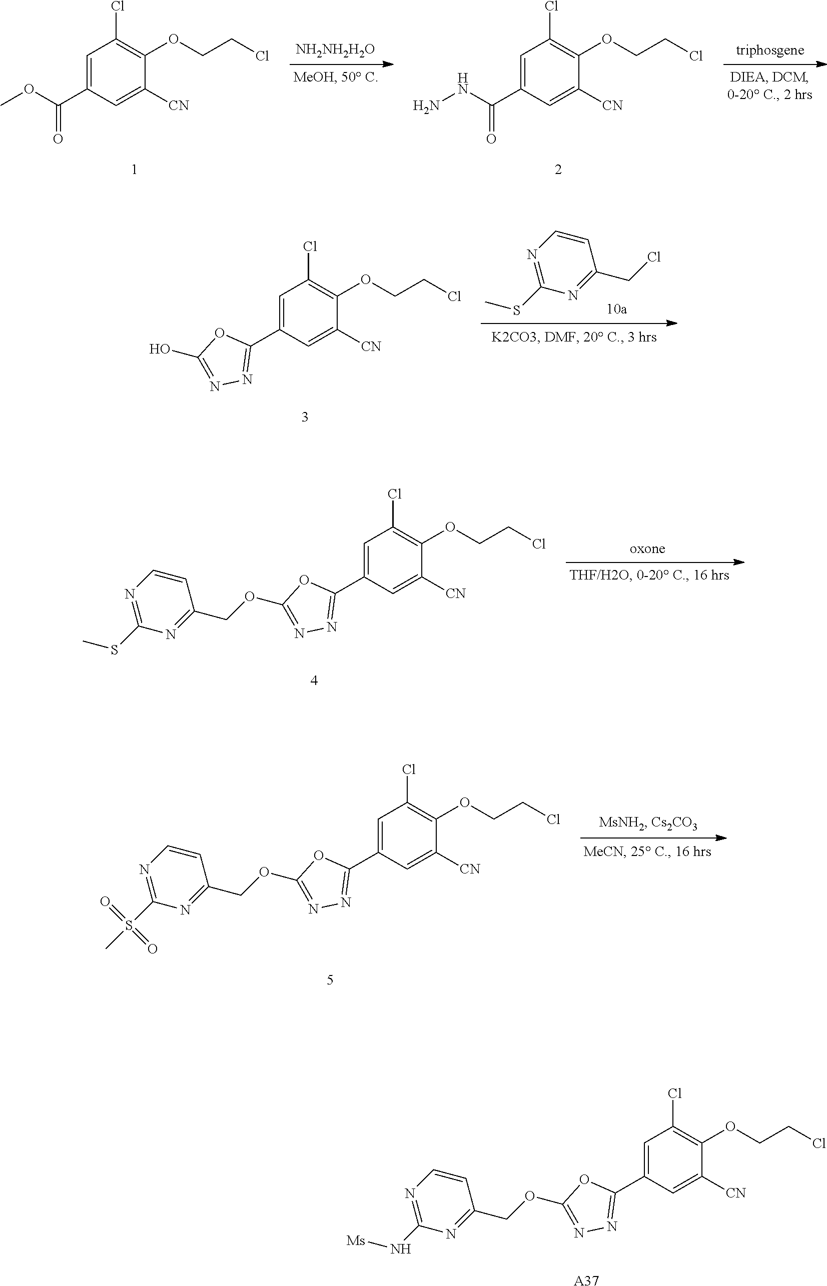

3-chloro-4-(2-chloroethoxy)-5-cyanobenzohydrazide (2): To a solution of methyl 3-chloro-4-(2-chloroethoxy)-5-cyano-benzoate (1) (5 g, 18.2 mmol) in MeOH (50 mL) was added NH₂NH₂H₂O (85.0% purity, 5.21 mL, 91.21 mmol) at 20° C. The mixture was stirred for 3 hrs at 50° C. under N₂. LCMS showed the starting material was consumed. The reaction was quenched with H₂O (100 mL) (lot of solid appeared), then filtered and the filter cake was dried under reduced pressure to give 3-chloro-4-(2-chloroethoxy)-5-cyano-benzohydrazide (2) (3.7 g, yield: 66.6%) as yellow solid. ¹H NMR (400 MHz, DMSO-d₆) δ=9.97 (s, 1H), 8.23 (d, J=2.0 Hz, 1H), 8.18 (d, J=2.0 Hz, 1H), 4.59 (s, 1H), 4.54 (t, J=4.8 Hz, 2H), 3.98 (t, J=4.8 Hz, 1H).

3-chloro-2-(2-chloroethoxy)-5-(5-hydroxy-1,3,4-oxadiazol-2-yl)benzonitrile (3): To a solution of 3-chloro-4-(2-chloroethoxy)-5-cyano-benzohydrazide (2) (1.00 g, 3.65 mmol) and DIEA (0.937 mL, 5.47 mmol) in DCM (10 mL) was added bis(trichloromethyl) carbonate (1.30 g, 4.38 mmol) at 0° C. The mixture was stirred for 1 h at 20° C. under N₂. TLC showed the reaction was completed. The reaction was quenched with H₂O (20 mL) and extracted with DCM (20 mL×3). The combined organic layer was washed with brine (20 mL), dried over Na₂SO₄, filtered and concentrated. The residue was purified by silica gel column chromatography to give 3-chloro-2-(2-chloroethoxy)-5-(5-hydroxy-1,3,4-oxadiazol-2-yl) benzonitrile (3) (0.45 g, yield: 37%) as yellow solid. ¹H NMR (400 MHz, DMSO-d₆) δ=12.84 (s, 1H), 8.18 (d, J=2.0 Hz, 1H), 8.16 (d, J=2.0 Hz, 1H), 4.56 (t, J=4.8 Hz, 2H), 3.99 (t, J=4.8 Hz, 2H).

3-chloro-2-(2-chloroethoxy)-5-(5-((2-(methylthio)pyrimidin-4-yl)methoxy)-1,3,4-oxadiazol-2-yl)benzonitrile (4): To a solution of 3-chloro-2-(2-chloroethoxy)-5-(5-hydroxy-1,3,4-oxadiazol-2-yl)benzonitrile (0.399 g, 1.33 mmol) (3) and K₂CO₃ (1.02 g, 7.38 mmol) in DMF (5 mL) was added 4-(chloromethyl)-2-methylsulfanyl-pyrimidine (0.310 g, 1.48 mmol) (10a) at 20° C. The mixture was stirred at the same temperature for 2 hrs. TLC showed the reaction was completed and the resulting mixture was quenched with H₂O (10 mL). The aqueous layer was extracted with EtOAc (15 mL×3). The combined organic layers were washed with brine (15 mL×5), dried over Na₂SO₄, filtered and concentrated under reduced pressure to give 3-chloro-2-(2-chloroethoxy)-5-(5-((2-methylsulfanylpyrimidin-4-yl)methoxy)-1,3,4-oxadiazol-2-yl) benzonitrile (4) (0.430 g, yield: 66.5%) as yellow oil. LCMS (220 nm): 52.0% purity. Exact Mass: 437.01; found 438.0, 440.0.

3-chloro-2-(2-chloroethoxy)-5-(5-((2-(methylsulfonyl)pyrimidin-4-yl)methoxy)-1,3,4-oxadiazol-2-yl)benzonitrile (5): To a solution of 3-chloro-2-(2-chloroethoxy)-5-(5-((2-methylsulfanylpyrimidin-4-yl)methoxy)-1,3,4-oxadiazol-2-yl)benzonitrile (4) (430 mg, 0.98 mmol) in THF (5 mL) and H₂O (5 mL) was added Oxone (1.51 g, 2.45 mmol) at 0° C. The mixture was stirred at 20° C. for 16 hrs. TLC showed the reaction was completed and the resulting mixture was quenched with sat. Na₂SO₃ (10 mL). The aqueous layer was extracted with EtOAc (10 mL×3). The combined organic layers were washed with brine (10 mL×2), dried over Na₂SO₄, filtered and concentrated under reduced pressure to give 3-chloro-2-(2-chloroethoxy)-5-(5-((2-(methylsulfonyl)pyrimidin-4-yl)methoxy)-1,3,4-oxadiazol-2-yl) benzonitrile (5) (0.420 g, yield: 91.0%) as yellow oil. The crude product was used next step directly. ¹H NMR (400 MHz, CDCl₃) δ=8.97 (d, J=5.2 Hz, 1H), 8.09 (d, J=2.4 Hz, 1H), 7.98 (d, J=2.0 Hz, 1H), 7.53 (d, J=5.2 Hz, 1H), 5.24 (s, 2H), 4.60 (t, J=5.6 Hz, 2H), 3.92 (t, J=5.2 Hz, 2H), 3.37 (s, 3H)

N-(4-(((5-(3-chloro-4-(2-chloroethoxy)-5-cyanophenyl)-1,3,4-oxadiazol-2-yl)oxy)methyl) pyrimidin-2-yl)methanesulfonamide (A37): To a solution of 3-chloro-2-(2-chloroethoxy)-5-(5-((2-(methylsulfonyl)pyrimidin-4-yl)methoxy)-1,3,4-oxadiazol-2-yl)benzonitrile (5) (50.6 mg, 0.53 mmol) in MeCN (2 mL) was added Cs₂CO₃ (173 mg, 0.53 mmol) and Methane sulfonamide (0.0506 g, 0.532 mmol) and the mixture was stirred at 20° C. for 16 hrs. LCMS showed the reaction was completed and the resulting mixture was quenched with H₂O (5 mL). The aqueous layer was extracted with EtOAc (5 mL×3). The combined organic layers were washed with brine (5 mL×2), dried over Na₂SO₄, filtered and concentrated under reduced pressure. The residue was purified by p-HPLC (FA) to give N-(4-(((5-(3-chloro-4-(2-chloroethoxy)-5-cyanophenyl)-1,3,4-oxadiazol-2-yl)oxy)methyl)pyrimidin-2-yl)methanesulfonamide (A37) (14.5 mg, yield: 14.1%) as white solid. ¹H NMR (400 MHz, CCl₃) δ=8.61 (d, J=5.2 Hz, 1H), 8.07 (d, J=2.0 Hz, 1H), 7.97 (d, J=2.0 Hz, 1H), 7.01 (d, J=5.2 Hz, 1H), 5.06 (s, 2H), 4.57 (t, J=6.0 Hz, 2H), 3.91 (t, J=6.0 Hz, 2H), 3.39 (s, 3H). LCMS (220 nm): 95.4%. Exact Mass: 484.01; found 485.0, 487.0.

### Example 13: Synthesis of N-(4-((2-(2-(3-chloro-4-(2-chloroethoxy)-5-cyanophenyl)propan-2-yl)phenoxy)methyl)pyrimidin-2-yl)methanesulfonamide (Compound A39)

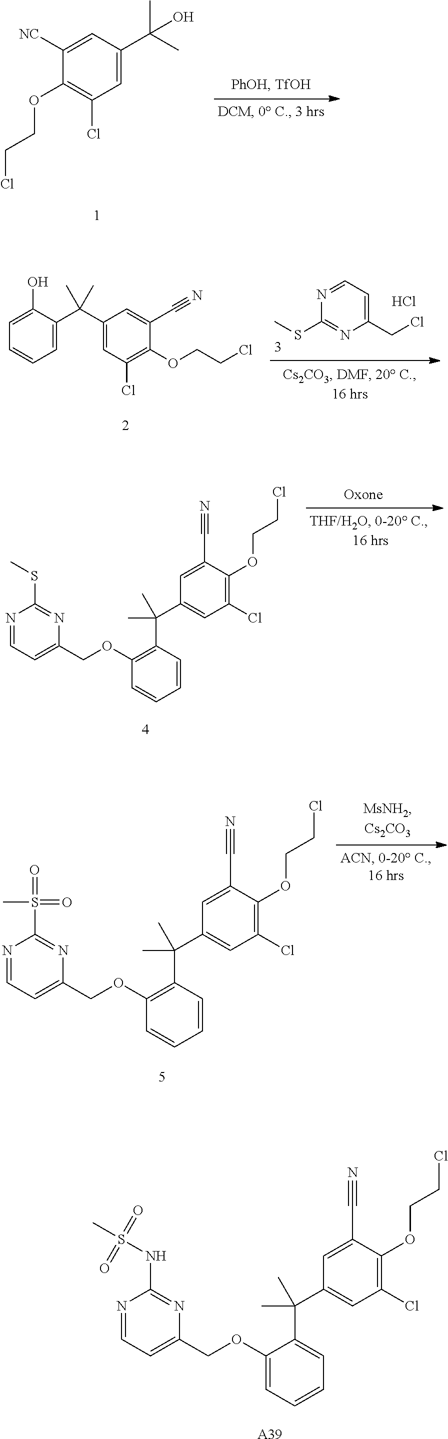

3-chloro-2-(2-chloroethoxy)-5-(2-(2-hydroxyphenyl)propan-2-yl)benzonitrile (2): To a solution of 3-chloro-2-(2-chloroethoxy)-5-(2-hydroxypropan-2-yl)benzonitrile (1) (13.2 g, 0.0453 mol) in DCM (65 mL) was added phenol (6.39 g, 0.0679 mol), then TfOH (8.01 mL, 0.0905 mol) was added into the reaction drop wise at 0° C. The mixture was stirred at 0° C. for 3 hrs under N₂. The reaction was quenched with H₂O (100 mL) and extracted with EtOAc (50 mL×3). The combined organic layers were washed with brine (50 mL×3), dried over Na₂SO₄, filtered and concentrated. The crude product was purified by silica gel column to give 3-chloro-2-(2-chloroethoxy)-5-(2-(2-hydroxyphenyl)propan-2-yl)benzonitrile and phenol (1:1) (2) (2.90 g, yield: 18.3%) as yellow oil. ¹H NMR (400 MHz, CDCl₃) δ 7.50-7.40 (m, 2H), 7.34 (d, J=2.4 Hz, 1H), 7.21-7.10 (m, 1H), 7.05-6.94 (m, J=2.4 Hz, 1H), 6.66 (d, J=6.4 Hz, 1H), 4.41 (t, J=6.4 Hz, 2H), 3.87 (t, J=6.4 Hz, 2H), 1.68 (s, 6H).

3-chloro-2-(2-chloroethoxy)-5-(2-(2-((2-(methylthio)pyrimidin-4-yl)methoxy)phenyl)propan-2-yl)benzonitrile (4): To a solution of 3-chloro-2-(2-chloroethoxy)-5-[1-(2-hydroxyphenyl)-1-methyl-ethyl] benzonitrile (2) (0.2 g, 0.571 mmol) and 4-(chloromethyl)-2-(methylthio)pyrimidine (3) (0.144 g, 0.685 mmol) in DMF (2 mL) was added Cs₂CO₃ (0.465 g, 1.43 mol) at 20° C. and stirred at 20° C. for 16 hrs under N₂. The mixture was quenched with water (5 mL) and extracted with EtOAc (5 mL×3), the combined organic layers were washed with brine (5 mL×2), dried over Na₂SO₄, filtered and concentrated under reduced pressure to give 3-chloro-2-(2-chloroethoxy)-5-(2-(2-((2-(methylthio) pyrimidin-4-yl)methoxy)phenyl)propan-2-yl)benzonitrile (4) (0.23 g, yield: 82.5%) as yellow oil. δ 8.46 (d, J=5.2 Hz, 1H), 7.52 (d, J=8.0 Hz, 1H), 7.44 (s, 1H), 7.31-7.29 (m, 1H), 7.10 (t, J=7.6 Hz, 1H), 6.81 (d, J=8.0 Hz, 1H), 6.41 (d, J=4.8 Hz, 1H), 4.74 (s, 2H), 4.33 (t, J=6.0 Hz, 2H), 3.85 (t, J=6.0 Hz, 2H), 2.55 (s, 3H), 1.70 (s, 6H).

3-chloro-2-(2-chloroethoxy)-5-(2-(2-((2-(methylsulfonyl)pyrimidin-4-yl)methoxy)phenyl)propan-2-yl)benzonitrile (5): To a solution of 3-chloro-2-(2-chloroethoxy)-5-(2-(2-((2-(methylthio)pyrimidin-4-yl)methoxy)phenyl)propan-2-yl)benzonitrile (4) (0.5 g, 1.02 mmol) in THF (5 mL) was added H₂O (5 mL) and Oxone (1.57 g, 2.56 mmol) at 0° C. and stirred at 20° C. for 16 hrs. The mixture was quenched with Na₂SO₃ (5 mL) and extracted with EtOAc (5 mL×2), the organic phase was washed with brine (5 mL×2), dried over Na₂SO₄, filtered and concentrated to give 3-chloro-2-(2-chloroethoxy)-5-(2-(2-((2-(methylsulfonyl)pyrimidin-4-yl) methoxy)phenyl)propan-2-yl)benzonitrile (5) (0.43 g, yield: 80.7%) as white solid. δ 8.85 (d, J=5.2 Hz, 1H), 7.53 (d, J=7.6 Hz, 1H), 7.45 (s, 1H), 7.46-7.29 (m, 1H), 7.28 (s, 1H), 7.20-7.08 (m, 1H), 7.00 (d, J=5.2 Hz, 1H), 6.80 (d, J=7.6 Hz, 1H), 4.95 (s, 2H), 4.35 (t, J=6.0 Hz, 2H), 3.85 (t, J=6.0 Hz, 2H), 3.40 (s, 3H), 1.70 (s, 6H).

N-(4-((2-(2-(3-chloro-4-(2-chloroethoxy)-5-cyanophenyl)propan-2-yl)phenoxy)methyl)pyrimidin-2-yl)methanesulfonamide (A39): To a solution of 3-chloro-2-(2-chloroethoxy)-5-(2-(2-((2-(methylsulfonyl)pyrimidin-4-yl)methoxy)phenyl)propan-2-yl)benzonitrile (0.4 g, 0.769 mmol) in MeCN (4 mL) was added methanesulfonamide (0.219 g, 2.31 mmol) and Cs₂CO₃ (0.751 g, 2.31 mmol) at 0° C. and stirred at 20° C. for 16 hrs. The mixture was quenched with water (5 mL) and extracted with EtOAc (5 mL×2), the combined organic layers were washed with brine (5 mL×2), dried over Na₂SO₄, filtered and concentrated to give N-(4-((2-(2-(3-chloro-4-(2-chloroethoxy)-5-cyanophenyl)propan-2-yl)phenoxy)methyl)pyrimidin-2-yl)methanesulfonamide (A39) (180 mg, yield: 42.3%) as yellow solid. ¹H NMR (400 MHz, CDCl₃) δ 8.68 (brs, 1H), 8.56 (d, J=5.2 Hz, 1H), 7.53 (d, J=7.6 Hz, 1H), 7.43 (d, J=2.4 Hz, 1H), 7.36-7.28 (m, 1H), 7.26 (d, J=2.4 Hz, 1H), 7.17-7.06 (m, 1H), 6.84 (d, J=7.6 Hz, 1H), 6.52 (d, J=5.2 Hz, 1H), 4.74 (s, 2H), 4.34 (t, J=6.0 Hz, 2H), 3.85 (t, J=6.0 Hz, 2H), 3.45 (s, 3H), 1.70 (s, 6H). LCMS: (220 nm): 98.5%. Exact Mass: 534.1; found 535.3/537.2.

### Example 14: Synthesis of N-(4-((1-(3-chloro-4-(2-chloroethoxy)-5-cyanobenzoyl)piperidin-4-yl)oxy) pyrimidin-2-yl)methanesulfonamide (Compound A6)

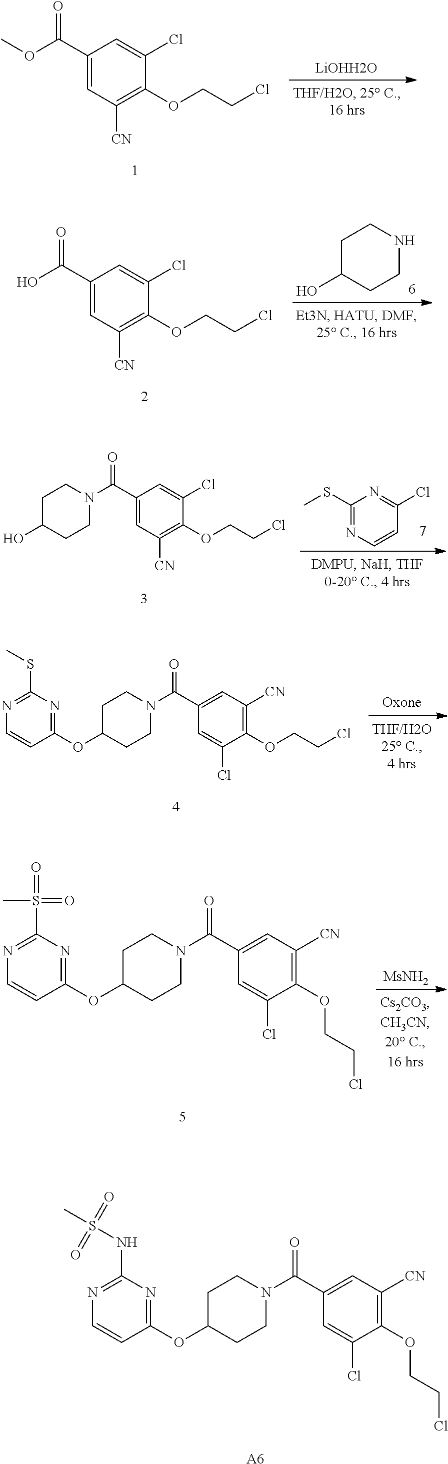

3-chloro-4-(2-chloroethoxy)-5-cyanobenzoic acid (2): To a solution of methyl 3-chloro-4-(2-chloroethoxy)-5-cyano-benzoate (1) (5.00 g, 18.2 mmol) in THF/H₂O (62.5 mL, v:v=4/1) was added LiOH H₂O (1.53 g, 36.5 mmol) at 25° C. and the mixture was stirred at 25° C. for 16 hrs. The mixture was quenched with water (50 mL) and extracted with EtOAc (50 mL×3). The combined organic layers were washed with brine (100 mL×2), dried over Na₂SO₄, filtered and concentrated under reduced pressure to give a residue. The residue was purified by silica gel column to give 3-chloro-4-(2-chloroethoxy)-5-cyano-benzoic acid (2) (4.00 g, yield: 82.6%) as white solid. ¹H NMR (400 MHz, CDCl₃) δ=8.42-8.18 (m, 2H), 4.62 (t, J=5.8 Hz, 2H), 3.92 (t, J=5.8 Hz, 2H).

3-chloro-2-(2-chloroethoxy)-5-(4-hydroxypiperidine-1-carbonyl)benzonitrile (3): To a solution of 5-chloro-4-(2-chloroethoxy)-3-cyano-cyclohexa-1,5-diene-1-carboxylic acid (2) (1.2 g, 4.58 mmol), piperidin-4-ol (0.926 g, 9.16 mmol) and HATU (2.61 g, 6.87 mmol) in DMF (12 mL) was added TEA (1.91 mL, 0.0137 mol) and the mixture was stirred at 25° C. for 16 hrs. The mixture was quenched with H₂O (10 mL) and extracted with EtOAc (10 mL×3). The combined organic layers were washed with brine (10 mL×2), dried over Na₂SO₄, filtered and concentrated under reduced pressure to give 3-chloro-2-(2-chloroethoxy)-5-(4-hydroxypiperidine-1-carbonyl)benzonitrile (3) (0.218 g, yield: 58.3%) as yellow oil. ¹H NMR (400 MHz, DMSO-d₆) δ=7.89-7.87 (m, 2H), 4.79-4.780 (m, 1H), 4.51-4.49 (m, 2H), 4.00-3.97 (m, 2H), 3.74 (s, 1H), 3.45 (s, 1H), 3.13 (s, 2H), 1.76 (s, 2H), 1.38 (s, 2H).

3-chloro-2-(2-chloroethoxy)-5-(4-((2-(methylthio)pyrimidin-4-yl)oxy)piperidine-1-carbonyl)benzonitrile (4): To a mixture of 3-chloro-2-(2-chloroethoxy)-5-(4-hydroxypiperidine-1-carbonyl)benzonitrile (3) (0.500 g, 1.46 mmol), 4-chloro-2-methylsulfanyl-4,5-dihydropyrimidine (6) (0.474 g, 2.91 mmol) and DMPU (0.746 g, 5.83 mmol) in THF (5 mL) was added NaH (60.0% purity, 0.146 g, 3.64 mmol) in portions at 0° C. under N₂, then the mixture was stirred at 20° C. for 4 hrs. The mixture was quenched with aq. NH₄C₁ (10 mL) and extracted with EtOAc (10 mL×3). The combined organic layers were washed with brine (10 mL), dried over Na₂SO₄, filtered and concentrated under reduced pressure to give the crude product, then the crude product was purified by silica gel column to give 3-chloro-2-(2-chloroethoxy)-5-(4-((2-(methylthio)pyrimidin-4-yl)oxy) piperidine-1-carbonyl)benzonitrile (4) (0.240 g, yield: 35.2%) as yellow oil. ¹H NMR (400 MHz, CDCl₃) δ=8.27 (d, J=5.6 Hz, 1H), 7.71 (d, J=1.6 Hz, 1H), 7.58 (d, J=2.0 Hz, 1H), 6.40 (d, J=5.6 Hz, 1H), 5.48-5.45 (quint, J=7.0 Hz, 1H), 4.54-4.51 (t, J=6.0, 2H), 3.92-3.43 (m, 6H), 2.54 (s, 3H), 2.06-1.89 (m, 4H).

3-chloro-2-(2-chloroethoxy)-5-(4-((2-(methylsulfonyl)pyrimidin-4-yl)oxy)piperidine-1-carbonyl)benzonitrile (5): To a mixture of 3-chloro-2-(2-chloroethoxy)-5-(4-((2-(methylthio)pyrimidin-4-yl)oxy)piperidine-1-carbonyl)benzonitrile (4) (0.120 g, 0.257 mmol) in THF (1 mL) and H₂O (1 mL) was added Oxone (0.474 g, 0.770 mmol) at 25° C. under N₂, then the mixture was stirred at 25° C. for 4 hrs. The mixture was added into H₂O (10 mL) and extracted with EtOAc (5 mL×3). The combined organic layers were washed with brine (10 mL), dried over Na₂SO₄, filtered and concentrated under reduced pressure to give the crude product, then the crude was purified by p-TLC to give 3-chloro-2-(2-chloroethoxy)-5-(4-((2-(methylsulfonyl)pyrimidin-4-yl)oxy)piperidine-1-carbonyl)benzonitrile (5) (72 mg crude) as colorless oil.

N-(4-((1-(3-chloro-4-(2-chloroethoxy)-5-cyanobenzoyl)piperidin-4-yl)oxy)pyrimidin-2-yl)methanesulfonamide (A6): To a mixture of 3-chloro-2-(2-chloroethoxy)-5-(4-((2-(methylsulfonyl)pyrimidin-4-yl)oxy)piperidine-1-carbonyl)benzonitrile (5) (72 mg, 0.145 mmol) in CH₃CN (2 mL) was added Methanesulfonamide (41.1 mg, 0.433 mmol) and Cs₂CO₃ (141 mg, 0.433 mmol) at 20° C. under N₂, then the mixture was stirred at 20° C. for 16 hrs. The mixture was poured into H₂O (10 mL) and extracted with EtOAc (5 mL×3). The combined organic layers were washed with brine (10 mL), dried over Na₂SO₄, filtered and concentrated under reduced pressure to give the crude product, then the crude product was purified by p-HPLC (FA) to give N-(4-((1-(3-chloro-4-(2-chloroethoxy)-5-cyanobenzoyl)piperidin-4-yl)oxy)pyrimidin-2-yl)methanesulfonamide (A6) (4.95 mg, yield: 6.07%) as white solid. ¹H NMR (400 MHz, CDCl₃) δ=8.23 (d, J=6.8 Hz, 1H), 7.71 (d, J=1.6 Hz, 1H), 7.57 (d, J=1.2 Hz, 1H), 6.31 (d, J=6.4 Hz, 1H), 5.42 (s, 1H), 4.55-4.52 (t, J=6.0 Hz, 2H), 3.93-3.43 (t, J=6.0 Hz, 2H), 3.71-3.45 (m, 3H), 3.28 (s, 3H), 2.1-1.95 (m, 4H). LCMS: (220 nm): 90.98%, Exact Mass: 513.1; found 514.1/516.1.

### Example 15: Synthesis of N-[6-(4-{2-[3-chloro-4-(2-chloroethoxy)-5-cyanophenyl]propan-2-yl}phenyl)quinazolin-2-yl]methanesulfonamide (Compound A51)

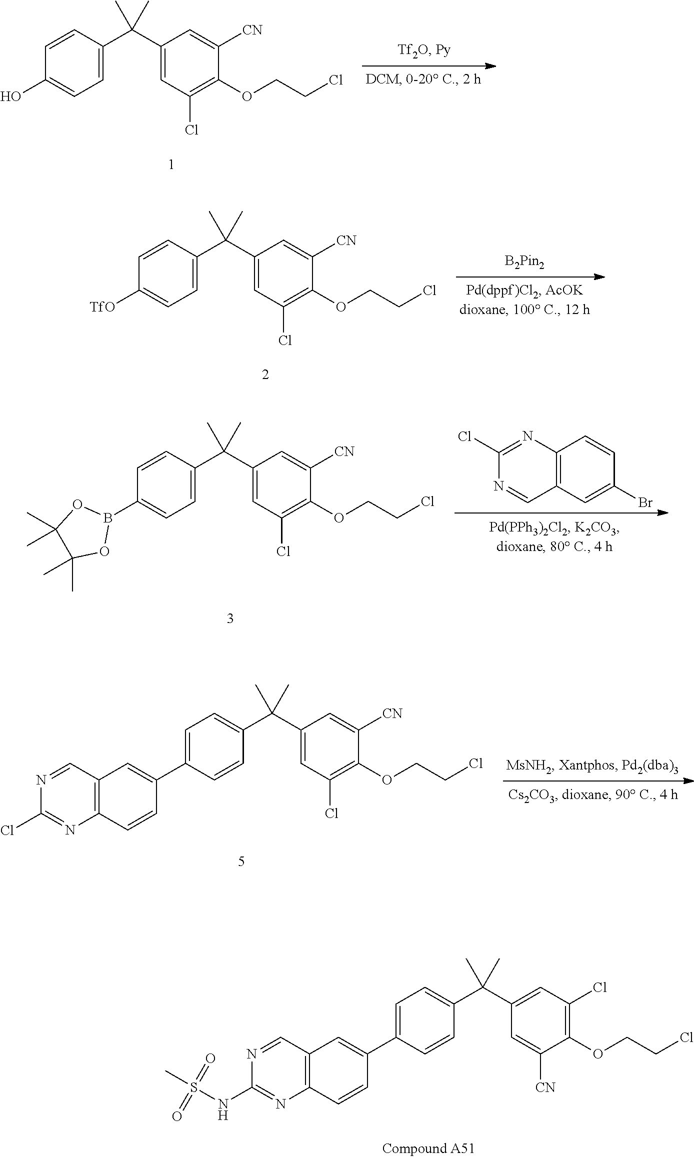 [SMILES: CC(C)(c1ccc(O)cc1)c1cc(Cl)c(OCCCl)c(C#N)c1 ; CC(C)(c1ccc(OS(=O)(=O)C(F)(F)F)cc1)c1cc(Cl)c(OCCCl)c(C#N)c1 ; CC(C)(c1ccc(B2OC(C)(C)C(C)(C)O2)cc1)c1cc(Cl)c(OCCCl)c(C#N)c1 ; Clc1ncc2cc(Br)ccc2n1 ; CC(C)(c1ccc(-c2ccc3nc(Cl)ncc3c2)cc1)c1cc(Cl)c(OCCCl)c(C#N)c1 ; CC(C)(c1ccc(-c2ccc3nc(NS(C)(=O)=O)ncc3c2)cc1)c1cc(Cl)c(OCCCl)c(C#N)c1]

4-(2-(3-chloro-4-(2-chloroethoxy)-5-cyanophenyl)propan-2-yl)phenyl trifluoromethanesulfonate (2): To a solution of 3-chloro-2-(2-chloroethoxy)-5-(2-(4-hydroxyphenyl)propan-2-yl)benzonitrile (1) (10.0 g, 28.6 mmol) in DCM (100 mL) was added pyridine (4.5 g, 57.1 mmol) and Trifluoromethanesulfonic anhydride (8.8 g, 31.4 mmol) at 0° C. Then the mixture was stirred at 20° C. for 2 hrs. LCMS showed the reaction was completed. The mixture was poured into H₂O (100 mL) and extracted with DCM (100 mL×3). The organic layers were combined and washed with brine (100 mL), dried over anhydrous Na₂SO₄, filtered and concentrated under reduced pressure. The residue was purified by silica gel column chromatography to give 4-(2-(3-chloro-4-(2-chloroethoxy)-5-cyanophenyl)propan-2-yl)phenyl (2) (12.0 g, yield: 87.1%) as yellow oil. ¹H-NMR (400 MHz, CDCl₃) δ=7.46 (d, J=2.4 Hz, 1H), 7.37 (d, J=2.0 Hz, 1H), 7.31-7.26 (m, 4H), 4.48 (t, J=6.0 Hz, 2H), 3.92 (t, J=6.0 Hz, 2H), 1.72 (s, 6H).

3-chloro-2-(2-chloroethoxy)-5-(2-(4-(4,4,5,5-tetramethyl-1,3,2-dioxaborolan-2-yl)phenyl)propan-2-yl)benzonitrile (3): To a solution of 4-(2-(3-chloro-4-(2-chloroethoxy)-5-cyanophenyl)propan-2-yl)phenyl trifluoromethanesulfonate (2) (5.0 g, 10.4 mmol) and Pin₂B₂ (3.9 g, 15.6 mmol) in 1,4-dioxane (50 mL) was added AcOK (3.1 g, 31.1 mmol) and Pd(dppf)Cl₂ (0.7 g, 1.04 mmol) under N₂ atmosphere at 25° C. Then the resulting mixture was stirred at 100° C. for 16 hrs under N₂. LCMS showed the reaction was completed. The mixture was concentrated under reduced pressure. The residue was purified by silica gel column chromatography to give 3-chloro-2-(2-chloroethoxy)-5-(2-(4-(4,4,5,5-tetramethyl-1,3,2-dioxaborolan-2-yl)phenyl)propan-2-yl)benzonitrile (3) (4 g, yield: 75.5%) as white solid. ¹H-NMR (400 MHz, CDCl₃) δ=7.77 (d, J=8.0 Hz, 2H), 7.42 (d, J=2.4 Hz, 1H), 7.33 (d, J=2.4 Hz, 1H), 7.20 (d, J=8.4 Hz, 2H), 4.42 (t, J=6.0 Hz, 2H), 3.88 (t, J=6.0 Hz, 2H), 3.71 (s, 1H), 1.67 (s, 6H), 1.35 (s, 12H).

3-chloro-2-(2-chloroethoxy)-5-(2-(4-(2-chloroquinazolin-6-yl)phenyl)propan-2-yl)benzonitrile (5): To a mixture of 6-bromo-2-chloroquinazoline (4) (0.5 g, 2.05 mmol) in 1,4-dioxane (5 mL) and H₂O (1 mL) was added 3-chloro-2-(2-chloroethoxy)-5-(2-(4-(4,4,5,5-tetramethyl-1,3,2-dioxaborolan-2-yl)phenyl)propan-2-yl)benzonitrile (3) (1.04 g, 2.26 mmol), Cs₂CO₃ (2.01 g, 6.16 mmol) and Pd(PPh₃)₂Cl₂ (0.15 g, 0.21 mmol) at 20° C. The mixture was stirred at 80° C. for 4 hrs under N₂ atmosphere. LCMS showed the reaction was completed. The mixture was poured into H₂O (10 mL) and extracted with EtOAc (5 mL×3). The organic layers were combined and washed with brine (5 mL), dried over anhydrous Na₂SO₄, filtered and concentrated under reduced pressure. The residue was purified by silica gel column chromatography to give 3-chloro-2-(2-chloroethoxy)-5-(2-(4-(2-chloroquinazolin-6-yl) phenyl)propan-2-yl)benzonitrile (5) (0.6 g, yield: 40.6%) as yellow oil. ¹H-NMR (400 MHz, CDCl₃) δ=9.36 (s, 1H), 8.23 (dd, J=2.0, 8.8 Hz, 1H), 8.13 (d, J=2.0 Hz, 1H), 8.08 (d, J=8.8 Hz, 1H), 7.65 (d, J=8.4 Hz, 2H), 7.51 (d, J=2.0 Hz, 1H), 7.38 (d, J=2.4 Hz, 1H), 7.35 (d, J=8.4 Hz, 2H), 4.45 (t, J=6.4 Hz, 2H), 3.89 (t, J=6.0 Hz, 2H), 1.74 (s, 6H).

N-(6-(4-(2-(3-chloro-4-(2-chloroethoxy)-5-cyanophenyl)propan-2-yl)phenyl) quinazolin-2-yl)methanesulfonamide (Compound A51): A solution of 3-chloro-2-(2-chloroethoxy)-5-(2-(4-(2-chloroquinazolin-6-yl)phenyl)propan-2-yl)benzonitrile (5) (200 mg, 0.40 mmol) in 1,4-dioxane (4 mL) was added methanesulfonamide (115 mg, 1.21 mmol), Cs₂CO₃ (262 mg, 0.81 mmol), Xantphos (93 mg, 0.16 mmol) and Pd₂(dba)₃ (73 mg, 0.08 mmol) at 20° C., The mixture was stirred at 90° C. for 4 hrs under N₂ atmosphere. LCMS showed the reaction was completed. The mixture was poured into H₂O (10 mL) and extracted with EtOAc (10 mL×3). The organic layers were washed with brine (10 mL), dried over Na₂SO₄, filtered and concentrated under reduced pressure. The residue was purified by prep-HPLC (FA) to give N-(6-(4-(2-(3-chloro-4-(2-chloroethoxy)-5-cyanophenyl)propan-2-yl)phenyl)quinazolin-2-yl) methanesulfonamide (Compound A51) (56.1 mg, yield: 23.6%) as white solid. ¹H-NMR (400 MHz, CDCl₃) δ=9.42 (s, 1H), 8.16 (dd, J=2.0, 8.8 Hz, 1H), 8.08 (d, J=1.6 Hz, 1H), 7.97 (d, J=8.8 Hz, 1H), 7.65 (d, J=8.4 Hz, 2H), 7.52 (d, J=2.4 Hz, 1H), 7.39 (d, J=2.4 Hz, 1H), 7.35 (d, J=8.4 Hz, 2H), 4.45 (t, J=6.0 Hz, 2H), 3.90 (t, J=6.4 Hz, 2H), 3.61 (s, 3H), 1.74 (s, 6H). LCMS (220 nm): 98.76%. Exact Mass: 554.1; found 555.0/557.0.

### Example 16: Synthesis of N-(4-((4-(2-(3-chloro-4-(2-chloroethoxy)-5-cyanophenyl)propan-2-yl)phenoxy)methyl)-3-fluoropyridin-2-yl)methanesulfonamide (Compound A46)

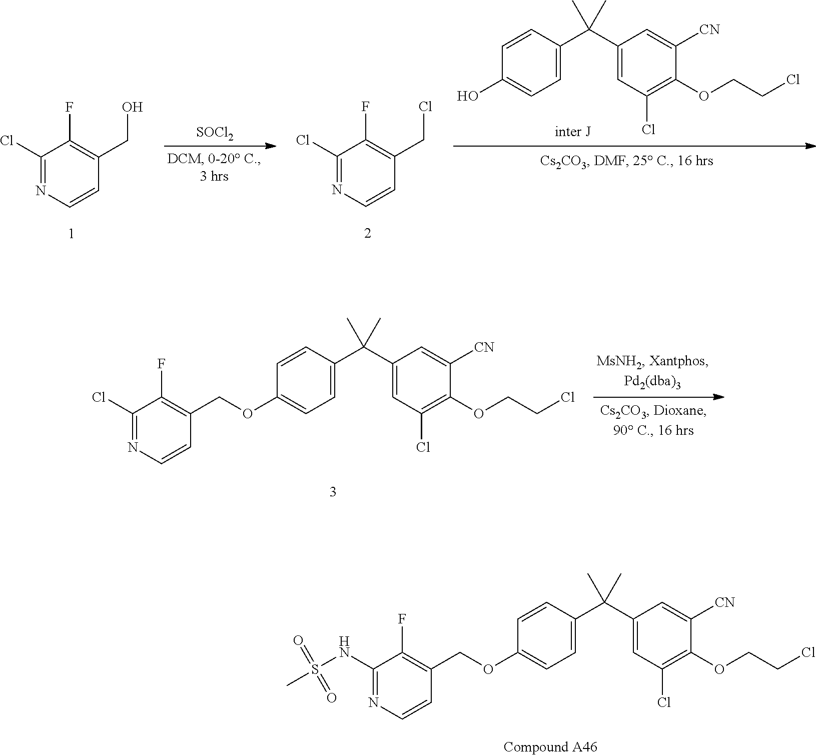

2-chloro-4-(chloromethyl)-3-fluoropyridine (2): To a solution of (2-chloro-3-fluoropyridin-4-yl)methanol (1) (5 g, 30 mmol) in DCM (50 mL) was added SOCl₂ (7 mL, 90 mmol) at 20° C. and the mixture was stirred at 20° C. for 3 hrs. LCMS showed the reaction was completed. The mixture was quenched with water (10 mL) and then extracted with DCM (50 mL×3). The combined organic layers were washed with brine (10 mL), dried over anhydrous Na₂SO₄, filtered and concentrated under reduced pressure to 2-chloro-4-(chloromethyl)-3-fluoropyridine (2) (5.4 g, yield: 87.2%) as light yellow oil. ¹H-NMR (400 MHz, CDCl₃) δ=8.24 (d, J=4.8 Hz, 1H), 7.39 (t, J=5.2 Hz, 1H), 4.63 (s, 2H).

3-chloro-5-(2-(4-((2-chloro-3-fluoropyridin-4-yl)methoxy)phenyl)propan-2-yl)-2-(2-chloroethoxy)benzonitrile (3): To a mixture of 2-chloro-4-(chloromethyl)-3-fluoropyridine (2) (5.4 g, 30 mmol) and Cs₂CO₃ (9.1 g, 90 mmol) in DMF (50 mL) was added 3-chloro-2-(2-chloroethoxy)-5-(2-(4-hydroxyphenyl)propan-2-yl)benzonitrile (inter J) (10.5 g, 30 mmol) at 20° C. and the mixture was stirred at 20° C. for 16 hrs. LCMS showed the reaction was completed. The mixture was quenched with water (100 mL) and extracted with EtOAc (50 mL×3), washed with brine (50 mL×2), dried over anhydrous Na₂SO₄, filtered and concentrated under reduced pressure. The residue was purified by silica gel column chromatography to give 3-chloro-5-(2-(4-((2-chloro-3-fluoropyridin-4-yl)methoxy)phenyl)propan-2-yl)-2-(2-chloroethoxy) benzonitrile (3) (12.0 g, yield: 72.9%) as colorless oil. ¹H-NMR (400 MHz, CDCl₃) δ=8.24 (d, J=5.2 Hz, 1H), 7.50 (t, J=4.8 Hz, 1H), 7.45 (d, J=2.4 Hz, 1H), 7.32 (d, J=2.4 Hz, 1H), 7.16-7.10 (m, 2H), 6.96-6.86 (m, 2H), 5.19 (s, 2H), 4.43 (t, J=6.4 Hz, 2H), 3.88 (t, J=6.0 Hz, 2H), 1.65 (s, 6H).

N-(4-((4-(2-(3-chloro-4-(2-chloroethoxy)-5-cyanophenyl)propan-2-yl)phenoxy)methyl)-3-fluoropyridin-2-yl)methanesulfonamide (Compound A46): To a mixture of 3-chloro-5-(2-(4-((2-chloro-3-fluoropyridin-4-yl)methoxy)phenyl)propan-2-yl)-2-(2-chloroethoxy)benzonitrile (3) (4.0 g, 10 mmol), methanesulfonamide (3.90 g, 40 mmol) and Cs₂CO₃ (5.3 g, 20 mmol) in 1,4-dioxane (50 mL) was added Xantphos (0.94 g, 1.6 mmol) and Tris (dibenzylideneacetone)dipalladium(0) (0.74 g, 0.81 mmol) at 20° C. and the mixture was stirred at 90° C. for 16 hrs under N₂ atmosphere. LCMS showed the reaction was completed. The mixture was poured into water (50 mL) and then extracted with EtOAc (50 mL×3). The combined organic layers were washed with brine (50 mL×2), dried over anhydrous Ns₂SO₄, filtered and concentrated under reduced pressure. The residue was purified by prep-HPLC (FA) to give N-(4-((4-(2-(3-chloro-4-(2-chloroethoxy)-5-cyanophenyl)propan-2-yl)phenoxy)methyl)-3-fluoropyridin-2-yl)methanesulfonamide (Compound A46) (1.34 g, yield: 29.6%) as yellow solid. ¹H-NMR (400 MHz, CDCl₃) δ=8.13 (br s, 1H), 7.45 (d, J=2.4 Hz, 1H), 7.32 (d, J=2.4 Hz, 1H), 7.22 (br t, J=4.8 Hz, 1H), 7.12 (d, J=2.0 Hz, 2H), 6.90 (d, J=8.8 Hz, 2H), 5.16 (s, 2H), 4.43 (t, J=6.0 Hz, 2H), 3.88 (t, J=6.0 Hz, 2H), 3.51 (s, 3H), 1.65 (s, 6H). LCMS: (220 nm):96.12%. Exact Mass:551.1; found 552.0/554.0.

### Example 17: Synthesis of N-(3-(((1-(3-chloro-4-(2-chloroethoxy)-5-cyanophenyl)-1H-indol-5-yl)oxy)methyl) bicyclo[1.1.1]pentan-1-yl)methanesulfonamide (Compound A57)

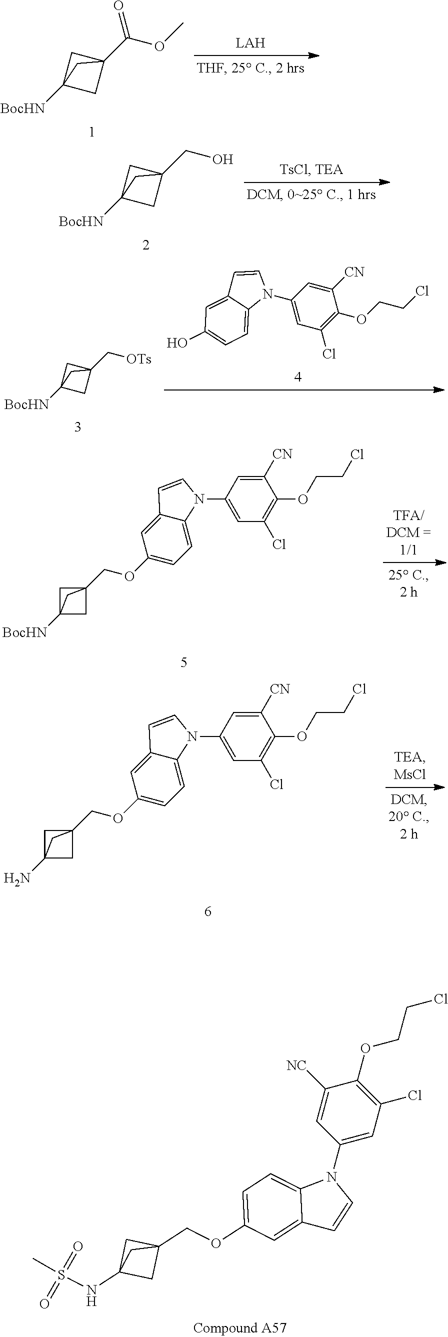

tert-butyl (3-(hydroxymethyl)bicyclo[1.1.1]pentan-1-yl)carbamate (2): To a solution of methyl 3-((tert-butoxycarbonyl)amino)bicyclo[1.1.1]pentane-1-carboxylate (1) (10.0 g, 41.4 mmol) in THF (100 mL) was added LiAlH₄ (2.4 g, 62.2 mmol) at 0° C. The mixture was stirred at 25° C. for 2 hrs under N₂ atmosphere. LCMS showed the reaction was completed. The mixture was quenched with Na₂SO₄.10H₂O and then the suspension was filtered and the filtrate was concentrated under reduced pressure to give tert-butyl (3-(hydroxymethyl)bicyclo[1.1.1]pentan-1-yl)carbamate (2) (8.0 g, yield: 90.5%) as white solid. ¹H-NMR (400 MHz, CDCl₃) δ=4.90 (br s, 1H), 3.63 (br d, J 4.8 Hz, 2H), 1.87 (br s, 6H), 1.38 (s, 9H).

(3-((tert-butoxycarbonyl)amino)bicyclo[1.1.1]pentan-1-yl)methyl 4-methylbenzene sulfonate (3): To a solution of tert-butyl (3-(hydroxymethyl)bicyclo[1.1.1]pentan-1-yl)carbamate (2) (8.0 g, 37.5 mmol) and TEA (7.8 mL, 56.3 mmol) in DCM (100 mL) was added 4-methylbenzenesulfonyl chloride (7.5 g, 39.4 mmol) at 0° C. The mixture was stirred at 25° C. for 1 h. LCMS showed the reaction was completed. The resulting mixture was quenched with water (50 mL). The aqueous layer was extracted with DCM (50 mL×3). The combined organic layers were washed with brine (50 mL), dried over Na₂SO₄, filtered and concentrated under reduced pressure. The residue was purified by silica gel column chromatography to give (3-((tert-butoxycarbonyl)amino)bicyclo[1.1.1]pentan-1-yl)methyl 4-methylbenzenesulfonate (3) (10.0 g, yield: 72.5%) as white solid. ¹H-NMR (400 MHz, CDCl₃) δ=7.78 (d, J=8.4 Hz, 2H), 7.35 (d, J=8.0 Hz, 2H), 4.11 (s, 2H), 2.46 (s, 3H), 1.93 (s, 6H), 1.43 (s, 9H).

tert-butyl (3-(((1-(3-chloro-4-(2-chloroethoxy)-5-cyanophenyl)-1H-indol-5-yl)oxy)methyl) bicyclo[1.1.1]pentan-1-yl)carbamate (5): To a solution of (3-((tert-butoxycarbonyl)amino) bicyclo[1.1.1]pentan-1-yl)methyl 4-methylbenzenesulfonate (3) (5.0 g, 13.6 mmol) and 3-chloro-2-(2-chloroethoxy)-5-(5-hydroxyindol-1-yl)benzonitrile (4) (4.3 g, 12.2 mmol) in DMF (50 mL) was added Cs₂CO₃ (6.6 g, 20.4 mmol) at 20° C. and the mixture was stirred at 20° C. for 12 hrs. LCMS showed the reaction was completed. The reaction was quenched with water (100 mL) and extracted with EtOAc (50 mL×3). The combined organic layers were washed with brine (50 mL×2), dried over Na₂SO₄, filtered and concentrated under reduced pressure. The residue was purified by silica gel column chromatography to give tert-butyl (3-(((1-(3-chloro-4-(2-chloroethoxy)-5-cyanophenyl)-1H-indol-5 yl)oxy)methyl)bicyclo[1.1.1]pentan-1-yl)carbamate (5) (4.5 g, yield: 61.0%) as yellow oil. ¹H-NMR (400 MHz, CDCl₃) δ=7.68-7.72 (m, 1H), 7.56 (d, J=2.6 Hz, 1H), 7.29 (d, J=8.8 Hz, 1H), 7.13 (d, J=3.2 Hz, 1H), 7.03 (d, J=2.4 Hz, 1H), 6.85 (dd, J=8.8, 2.38 Hz, 1H), 6.55 (d, J=3.2 Hz, 1H), 4.45 (t, J=6.0 Hz, 2H), 4.08 (s, 2H), 3.88 (t, J=6.0 Hz, 2H), 1.99 (s, 6H), 1.38 (s, 9H).

5-(5-((3-aminobicyclo[1.1.1]pentan-1-yl)methoxy)-1H-indol-1-yl)-3-chloro-2-(2-chloroethoxy)benzonitrile (6): To a solution of tert-butyl (3-(((1-(3-chloro-4-(2-chloroethoxy)-5-cyanophenyl)-1H-indol-5-yl)oxy)methyl)bicyclo[1.1.1]pentan-1-yl)carbamate (5) (4.5 g, 8.30 mmol) in DCM (24 mL) was added TFA (24 mL) at 0° C., The mixture was stirred at 25° C. for 2 hrs. TLC showed the reaction was completed and the resulting mixture was quenched with water (10 mL). The aqueous layer was extracted with DCM (10 mL×3). The combined organic layers were washed with brine (10 mL×2), dried over anhydrous Na₂SO₄, filtered and concentrated under reduced pressure to give 5-(5-((3-aminobicyclo[1.1.1]pentan-1-yl)methoxy)-1H-indol-1-yl)-3-chloro-2-(2-chloroethoxy)benzonitrile (6) (3.0 g, yield: 81.8%) as yellow oil which was used directly for next step without further purification. ¹H-NMR (400 MHz, CDCl₃) δ=7.77 (d, J=2.6 Hz, 1H), 7.63 (d, J=2.6 Hz, 1H), 7.36 (d, J=8.8 Hz, 1H), 7.22 (d, J=3.2 Hz, 1H), 7.09 (d, J=2.4 Hz, 1H), 6.85 (dd, J=8.8, 2.38 Hz, 1H), 6.61 (d, J=3.2 Hz, 1H), 4.53 (t, J=6.0 Hz, 2H), 3.94 (t, J=6.0 Hz, 2H)

N-(3-(((1-(3-chloro-4-(2-chloroethoxy)-5-cyanophenyl)-1H-indol-5-yl)oxy)methyl) bicycle[1.1.1]pentan-1-yl)methanesulfonamide (Compound A57): To a solution of 5-(5-((3-aminobicyclo[1.1.1]pentan-1-yl)methoxy)-1H-indol-1-yl)-3-chloro-2-(2-chloroethoxy) benzonitrile (6) (4 g, 9.04 mmol) and TEA (5.0 mL, 36.2 mmol) in DCM (40 mL) was added a solution of MsCl (1.5 g, 12.8 mmol) in DCM (2 mL) at 0° C. and the mixture was stirred at 20° C. for 2 h. LCMS showed the reaction was completed. The mixture was quenched with water (50 mL) and extracted with DCM (20 mL×3). The combined organic layers were washed with brine (20 mL×2), dried over anhydrous Na₂SO₄, filtered and concentrated under reduced pressure. The residue was purified by silica gel column chromatography to give N-(3-(((1-(3-chloro-4-(2-chloroethoxy)-5-cyanophenyl)-1H-indol-5-yl)oxy)methyl)bicyclo[1.1.1]pentan-1-yl)methanesulfonamide (Compound A57) (1.0 g, yield: 21.5%) as white solid. ¹H-NMR (400 MHz, DMSO-d₆) δ=8.11-8.08 (m, 2H), 7.98 (s, 1H), 7.67 (d, J=3.2 Hz, 1H), 7.51 (d, J=8.8 Hz, 1H), 7.15 (d, J=2.4 Hz, 1H), 6.87 (dd, J=9.2, 2.4 Hz, 1H), 6.63 (d, J=3.2 Hz, 1H), 4.49 (t, J=5.2 Hz, 2H), 4.12 (s, 2H), 4.01 (t, J=5.2 Hz, 2H), 2.90 (s, 3H), 1.96 (s, 6H). LCMS:(220 nm): 100%. Exact Mass: 519.1; found 520.0/522.0.

### Example 18: Synthesis of N-(6-(4-(2-(3-chloro-4-(2-chloroethoxy)-5-cyanophenyl)propan-2-yl) phenyl)quinoxalin-2-yl)methanesulfonamide (Compound A107)

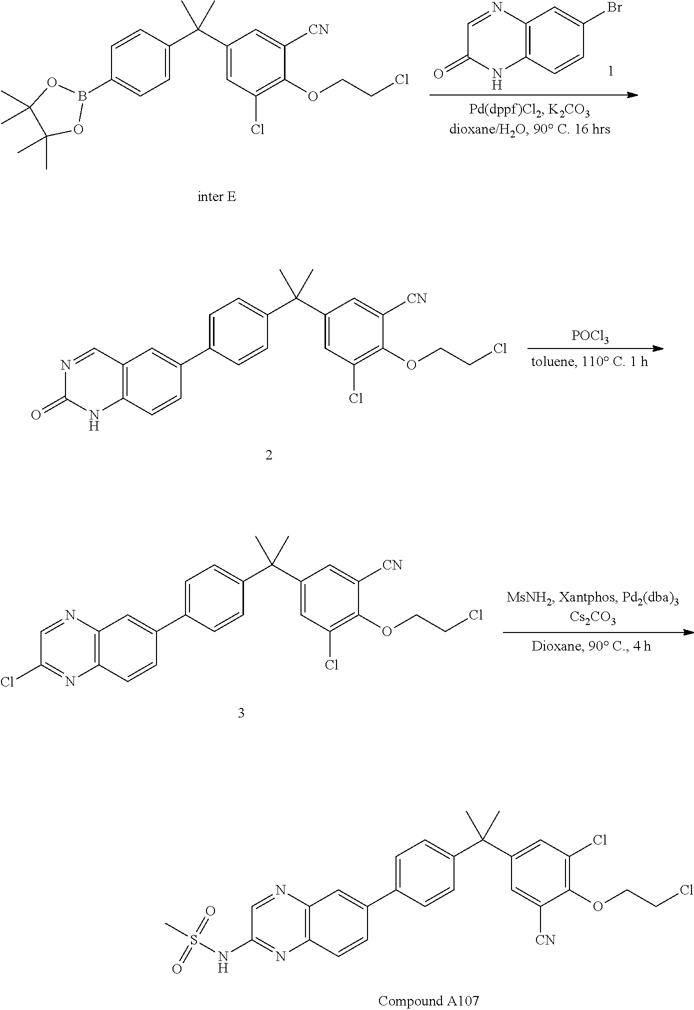

3-chloro-2-(2-chloroethoxy)-5-(2-(4-(2-chloroquinoxalin-6-yl)phenyl)propan-2-yl)benzonitrile (2): To a solution of 6-bromo-1H-quinoxalin-2-one (1) (300 mg, 1.3 mmol), 3-chloro-2-(2-chloroethoxy)-5-(2-(4-(4,4,5,5-tetramethyl-1,3,2-dioxaborolan-2-yl)phenyl)propan-2-yl)benzonitrile (inter E) (613 mg, 1.3 mmol) and K₂C₀₃ (368 mg, 2.70 mmol) in 1,4-dioxane (3 mL) and water (0.6 mL) was added Pd(dppf)Cl₂ (98 mg, 0.13 mmol) at 25° C. and the mixture was stirred at 90° C. for 16 hrs under N₂ atmosphere. LCMS showed the reaction was completed. The reaction mixture was diluted with H₂O (10 mL) and then extracted with EtOAc (10 mL×3). The combined organic layers were washed with brine (5 mL), dried over anhydrous Na₂SO₄, filtered and concentrated under reduced pressure. The residue was purified by silica gel column chromatography to give 3-chloro-2-(2-chloroethoxy)-5-(2-(4-(2-oxo-1,2-dihydroquinoxalin-6-yl)phenyl)propan-2-yl)benzonitrile (2) (180 mg, yield: 25.4%) as white solid. ¹H-NMR (400 MHz, CDCl₃) δ=8.40 (s, 1H), 8.13 (d, J=1.6 Hz, 1H), 7.87-7.77 (m, 1H), 7.62 (s, 1H), 7.60 (s, 1H), 7.51 (d, J=2.4 Hz, 1H), 7.39 (d, J=2.4 Hz, 1H), 7.31 (d, J=8.4 Hz, 2H), 4.45 (t, J=6.4 Hz, 2H), 3.89 (t, J=6.0 Hz, 2H), 1.73 (s, 6H).

3-chloro-2-(2-chloroethoxy)-5-(2-(4-(2-chloroquinoxalin-6-yl)phenyl)propan-2-yl)benzonitrile (3): A solution of 3-chloro-2-(2-chloroethoxy)-5-(2-(4-(2-oxo-1,2-dihydroquinoxalin-6-yl)phenyl)propan-2-yl)benzonitrile (2) (160 mg, 0.33 mmol) in POCl₃ (1 mL) and toluene (4 mL) was stirred at 110° C. for 1 h under N₂ atmosphere. TLC showed the reaction was completed. The mixture was concentrated and then adjusted to pH=8-9 with sat. NaHCO₃. The mixture was extracted with EtOAc (10 mL×2), and then the combined organic layers were washed with brine (10 mL), dried over anhydrous Na₂SO₄, filtered and concentrated under reduced pressure. The residue was purified by p-TLC to give 3-chloro-2-(2-chloroethoxy)-5-(2-(4-(2-chloroquinoxalin-6-yl)phenyl)propan-2-yl)benzonitrile (3) (90 mg, yield: 54.2%) as yellow oil. ¹H-NMR (400 MHz, CDCl₃) δ=8.82 (s, 1H), 8.32 (s, 1H), 8.10-8.08 (m, 1H), 7.71 (d, J=8.4 Hz, 2H), 7.52 (d, J=2.4 Hz, 1H), 7.40 (d, J=2.4 Hz, 1H), 7.35 (d, J=8.4 Hz, 2H), 4.45 (t, J=6.0 Hz, 2H), 3.89 (t, J=6.4 Hz, 2H), 1.74 (s, 6H).

N-(6-(4-(2-(3-chloro-4-(2-chloroethoxy)-5-cyanophenyl)propan-2-yl)phenyl)quinoxalin-2-yl)methanesulfonamide (Compound A107): To a solution of 3-chloro-2-(2-chloroethoxy)-5-(2-(4-(2-chloroquinoxalin-6-yl)phenyl)propan-2-yl)benzonitrile (3) (90 mg, 0.18 mmol) in 1,4-dioxane (2 mL) was added methanesulfonamide (17 mg, 0.18 mmol), Cs₂CO₃ (118 mg, 0.36 mol), Xantphos (21 mg, 0.03 mol) and Pd₂(dba)₃ (17 mg, 0.02 mmol) at 20° C. under N₂. The mixture was stirred at 90° C. under N₂ atmosphere for 4 hrs. LCMS showed the reaction was completed. The reaction mixture was diluted with H₂O (10 mL). The resulting aqueous mixture were extracted with EtOAc (5 mL×3). The combined organic layers were washed with brine (5 mL), dried over anhydrous Na₂SO₄, filtered and concentrated under reduced pressure. The residue was purified by prep-HPLC (FA) to give N-(6-(4-(2-(3-chloro-4-(2-chloroethoxy)-5-cyanophenyl)propan-2-yl)phenyl)quinoxalin-2-yl)methanesulfonamide (Compound A107) (21.9 mg, yield: 21.8%) as white solid. ¹H-NMR (400 MHz, CDCl₃) δ=8.43 (br s, 1H), 8.16 (s, 1H), 7.90 (br d, J=8.0 Hz, 1H), 7.63 (d, J=8.4 Hz, 2H), 7.51 (d, J=2.4 Hz, 1H), 7.39 (d, J=2.4 Hz, 1H), 7.32 (d, J=8.4 Hz, 2H), 4.45 (t, J=6.0 Hz, 2H), 3.89 (t, J=6.0 Hz, 2H), 3.26 (br s, 3H), 1.73 (s, 6H). LCMS (220 nm): 97.94%. Exact Mass: 554.09; found 554.9/556.9.

### Example 19: Synthesis of N-(6-(4-(3-chloro-4-(2-chloroethoxy)-5-cyanophenoxy)phenyl)quinazolin-2-yl)methanesulfonamide (Compound A116)

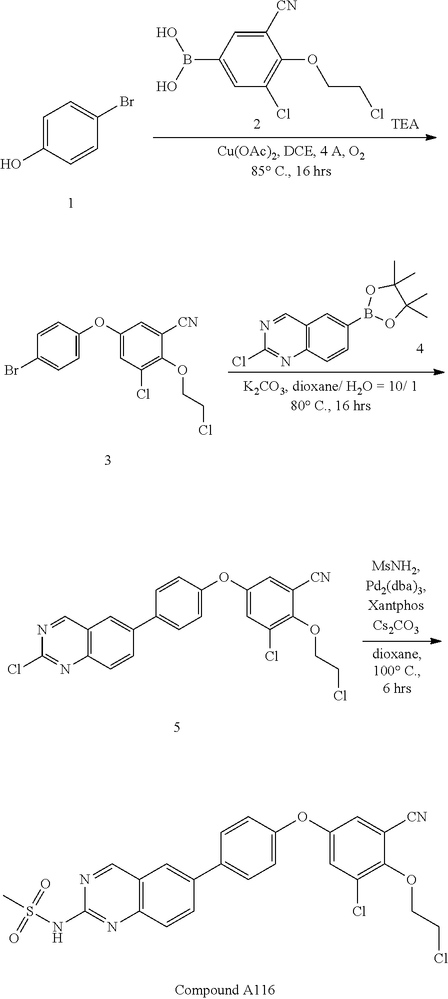 [SMILES: Oc1ccc(Br)cc1 ; N#Cc1cc(B(O)O)cc(Cl)c1OCCCl ; N#Cc1cc(Oc2ccc(Br)cc2)cc(Cl)c1OCCCl ; CC1(C)OB(c2ccc3nc(Cl)ncc3c2)OC1(C)C ; N#Cc1cc(Oc2ccc(-c3ccc4nc(Cl)ncc4c3)cc2)cc(Cl)c1OCCCl ; CS(=O)(=O)Nc1ncc2cc(-c3ccc(Oc4cc(Cl)c(OCCCl)c(C#N)c4)cc3)ccc2n1]

5-(4-bromophenoxy)-3-chloro-2-(2-chloroethoxy) benzonitrile (3): To a solution of (3-chloro-4-(2-chloroethoxy)-5-cyanophenyl)boronic acid (2) (300 mg, 1.16 mmol), 4-bromophenol (1) (200 mg, 1.16 mmol) and TEA (352 mg, 3.48 mmol) in DCE (2 mL) was added Cu(OAc)₂ (55 mg, 0.30 mmol). The mixture was stirred at 85° C. under 02 for 16 hrs. LCMS showed the reaction was completed. The reaction was quenched with water (10 mL) and extracted with DCM (5 mL×3). The combined organic layers were washed with brine (5 mL×2), dried over anhydrous Na₂SO₄, filtered and concentrated under reduced pressure. The residue was purified by silica gel column chromatography to give 5-(4-bromophenoxy)-3-chloro-2-(2-chloroethoxy)benzonitrile (3) (200 mg, yield: 25.5%) as yellow oil. ¹H-NMR (400 MHz, CDCl₃) δ=7.55-7.49 (m, 2H), 7.26 (d, J=2.8 Hz, 1H), 7.09 (d, J 3.2 Hz, 1H), 6.93-6.89 (m, 2H), 4.41 (t, J=6.0 Hz, 2H), 3.91-3.88 (m, 2H).

3-chloro-2-(2-chloroethoxy)-5-(4-(2-chloroquinazolin-6-yl)phenoxy)benzonitrile (5): To a mixture of 5-(4-bromophenoxy)-3-chloro-2-(2-chloroethoxy)benzonitrile (3) (170 mg, 0.44 mmol), 2-chloro-6-(4,4,5,5-tetramethyl-1,3,2-dioxaborolan-2-yl)quinazoline (4) (140 mg, 0.48 mmol) and dipotassium; carbonate (182 mg, 1.32 mmol) in 1,4-dioxane (2 mL) and H₂O (1 mL) was added Pd(dppf)Cl₂ (16 mg, 0.04 mmol) at 25° C. and the mixture was stirred at 80° C. for 16 hrs under N₂ atmosphere. LCMS showed the reaction was completed. The mixture was quenched with water (10 mL) and extracted with EtOAc (5 mL×3). The combined organic layers were washed with brine (5 mL), dried over Na₂SO₄, filtered and concentrated under reduced pressure. The residue was purified by silica gel column chromatography to give 3-chloro-2-(2-chloroethoxy)-5-(4-(2-chloroquinazolin-6-yl)phenoxy)benzonitrile (5) (70 mg, yield: 30.5%) as yellow solid. ¹H-NMR (400 MHz, CDCl₃) δ=9.41-9.35 (m, 1H) 8.21 (dd, J=8.8, 2.0 Hz, 1H), 8.14-8.06 (m, 2H), 7.76-7.70 (m, 2H), 7.19-7.18 (m, 1H), 7.17 (br s, 1H), 4.59-4.41 (m, 2H), 3.95-3.86 (m, 2H).

N-(6-(4-(3-chloro-4-(2-chloroethoxy)-5-cyanophenoxy)phenyl)quinazolin-2-yl) methanesulfonamide (Compound A116): To a solution of 3-chloro-2-(2-chloroethoxy)-5-(4-(2-chloroquinazolin-6-yl)phenoxy)benzonitrile (5) (50 mg, 0.11 mmol) in 1,4-dioxane (2 mL) was added Cs₂CO₃ (69 mg, 0.22 mmol), methanesulfonamide (50 mg, 0.53 mmol), Xantphos (25 mg, 0.02 mmol) and Pd₂(dba)₃ (20 mg, 0.01 mmol) at 20° C. under N₂. The mixture was stirred at 100° C. under N₂ atmosphere for 6 hrs. LCMS showed the reaction was completed. The reaction was quenched with water (10 mL) and then extracted with EtOAc (5 mL×3). The combined organic layers were washed with brine (5 mL), dried over anhydrous Na₂SO₄, filtered and concentrated under reduced pressure. The residue was purified by p-HPLC (neutral) to give N-(6-(4-(3-chloro-4-(2-chloroethoxy)-5-cyanophenoxy)phenyl)quinazolin-2-yl) methanesulfonamide (Compound A116) (12.9 mg, yield: 22.7%) as white solid. ¹H-NMR (400 MHz, DMSO-d₆) δ=9.51 (s, 1H), 8.36 (d, J=2.0 Hz, 1H), 8.27 (d, J=6.8 Hz, 1H), 7.89-7.84 (m, 3H), 7.66 (d, J=2.8 Hz, 1H), 7.62 (d, J=3.2 Hz, 1H), 7.24 (d, J=8.8 Hz, 2H), 4.42 (t, J=5.2 Hz, 2H), 3.98 (t, J=4.8 Hz, 2H), 3.44 (s, 3H). LCMS: (220 nm): 99.69%. Exact Mass: 528.04; found 528.9/529.9.

### Example 20: Synthesis of N-(1-(2-(1-(3-chloro-4-(2-chloroethoxy)-5-cyanophenyl)-1H-indol-5-yl)ethyl)azetidin-3-yl)methanesulfonamide (Compound A126)

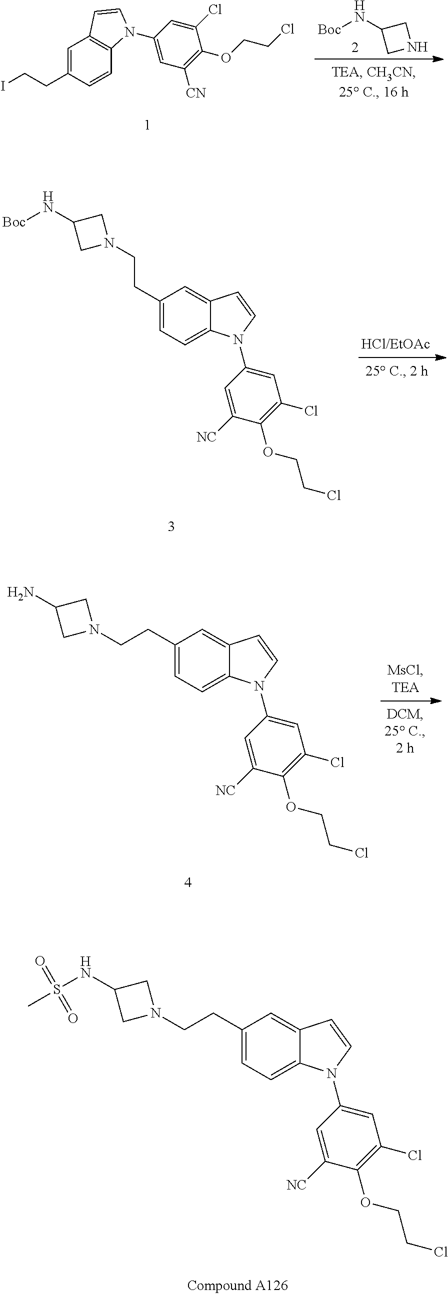 [SMILES: N#Cc1cc(-n2ccc3cc(CCI)ccc32)cc(Cl)c1OCCCl ; CC(C)(C)OC(=O)NC1CNC1 ; CC(C)(C)OC(=O)NC1CN(CCc2ccc3c(ccn3-c3cc(Cl)c(OCCCl)c(C#N)c3)c2)C1 ; N#Cc1cc(-n2ccc3cc(CCN4CC(N)C4)ccc32)cc(Cl)c1OCCCl ; CS(=O)(=O)NC1CN(CCc2ccc3c(ccn3-c3cc(Cl)c(OCCCl)c(C#N)c3)c2)C1]

tert-butyl (1-(2-(1-(3-chloro-4-(2-chloroethoxy)-5-cyanophenyl)-1H-indol-5-yl)ethyl) azetidin-3-yl)carbamate (3): To a solution of tert-butyl N-(azetidin-3-yl)carbamate (2) (80 mg, 0.47 mmol), 3-chloro-2-(2-chloroethoxy)-5-(5-(2-iodoethyl)-1H-indol-1-yl)benzonitrile (1) (225 mg, 0.47 mmol) and N,N-diethylethanamine (0.2 mL, 1.39 mmol) in DCM (2 mL) at 25° C. was stirred at 25° C. for 16 hrs. LCMS showed the reaction was completed. The mixture was quenched with water (5 mL) and extracted with DCM (3 mL×3). The combined organic layers were washed with brine (3 mL×2), dried over anhydrous Na₂SO₄, filtered and concentrated under reduce pressure. The residue was purified by silica gel column chromatography to give tert-butyl (1-(2-(1-(3-chloro-4-(2-chloroethoxy)-5-cyanophenyl)-1H-indol-5-yl)ethyl)azetidin-3-yl)carbamate (3) (150 mg, yield: 58.2%) as yellow oil. 6=7.78 (d, J=2.8 Hz, 1H), 7.74 (d, J=2.8 Hz, 1H), 7.51 (s, 1H), 7.40 (d, J=4.8 Hz, 1H), 7.23 (dd, J=8.8, 1.6 Hz, 1H), 7.20-7.18 (m, 1H), 6.71 (s, 1H), 4.52 (t, J=5.2 Hz, 2H), 4.45-4.16 (m, 4H), 3.94 (t, J=5.6 Hz, 2H), 3.83 (t, J=7.2 Hz, 2H), 3.05 (t, J=7.2 Hz, 2H).

5-(5-(2-(3-aminoazetidin-1-yl)ethyl)-1H-indol-1-yl)-3-chloro-2-(2-chloroethoxy) benzonitrile (4): A solution of tert-butyl tert-butyl (1-(2-(1-(3-chloro-4-(2-chloroethoxy)-5-cyanophenyl)-1H-indol-5-yl)ethyl)azetidin-3-yl)carbamate (3) (150 mg, 0.28 mmol) in HCl/EtOAc (5 mL, 4 M) was stirred at 25° C. for 2 h. LCMS showed the reaction was completed. The mixture was concentrated under reduced pressure to give 5-(5-(2-(3-aminoazetidin-1-yl)ethyl)-1H-indol-1-yl)-3-chloro-2-(2-chloroethoxy)benzonitrile (4) (120 mg, yield: 98.7%) as yellow solid. ¹H-NMR (400 MHz, MeOD) δ=7.98 (d, J=2.8 Hz, 1H), 7.88 (d, J=2.8 Hz, 1H), 7.62 (s, 1H), 7.58 (d, J=8.4 Hz, 1H), 7.54 (d, J=3.2 Hz, 1H), 7.22 (dd, J=8.8, 1.6 Hz, 1H), 6.74-6.69 (m, 1H), 4.56 (t, J=5.2 Hz, 2H), 4.45-4.16 (m, 5H), 3.97 (t, J=5.6 Hz, 2H), 3.68 (t, J=7.2 Hz, 2H), 3.08 (t, J=7.2 Hz, 2H).

N-(1-(2-(1-(3-chloro-4-(2-chloroethoxy)-5-cyanophenyl)-1H-indol-5-yl)ethyl)azetidin-3-yl)methanesulfonamide (Compound A126): To a solution of 5-(5-(2-(3-aminoazetidin-1-yl)ethyl)-1H-indol-1-yl)-3-chloro-2-(2-chloroethoxy)benzonitrile (4) (120 mg, 0.28 mmol) and TEA (0.16 mL, 1.12 mmol) in DCM (2 mL) was added MsCl (29 mg, 0.25 mmol) in DCM (2 mL) at 0° C. and the mixture was stirred at 25° C. for 2 h. LCMS showed the reaction was completed. The mixture was quenched with water (5 mL), extracted with DCM (5 mL×3). The combined organic layers were washed with brine (10 mL×2), dried over anhydrous Na₂SO₄, filtered and concentrated under reduce pressure. The residue was purified by p-HPLC (neutral) to give N-(1-(2-(1-(3-chloro-4-(2-chloroethoxy)-5-cyanophenyl)-1H-indol-5-yl)ethyl) azetidin-3-yl)methanesulfonamide (Compound A126) (37.8 mg, yield: 26.7%) as white solid. ¹H-NMR (400 MHz, DMSO-d₆) δ=8.13 (d, J=2.8 Hz, 1H), 8.10 (d, J=2.8 Hz, 1H), 7.68 (d, J=3.6 Hz, 1H), 7.56 (d, J=8.4 Hz, 2H), 7.52 (d, J=8.4 Hz, 1H), 7.08 (d, J=7.2 Hz, 1H), 6.66 (d, J=3.2 Hz, 1H), 4.50 (t, J=5.2 Hz, 2H), 4.01 (t, J=5.2 Hz, 2H), 3.89-3.82 (m, 1H), 3.61-3.57 (m, 2H), 2.85 (s, 3H), 2.83-2.79 (m, 2H), 2.66-2.60 (m, 4H). LCMS: (220 nm): 100%. Exact Mass: 506.1; found 507.0/509.1.

### Example 21: Synthesis of N-(3-((4-(2-(3-chloro-4-(2-chloroethoxy)-5-cyanophenyl)propan-2-yl)phenoxy)methyl)bicyclo[1.1.1]pentan-1-yl)methanesulfonamide (Compound A132)

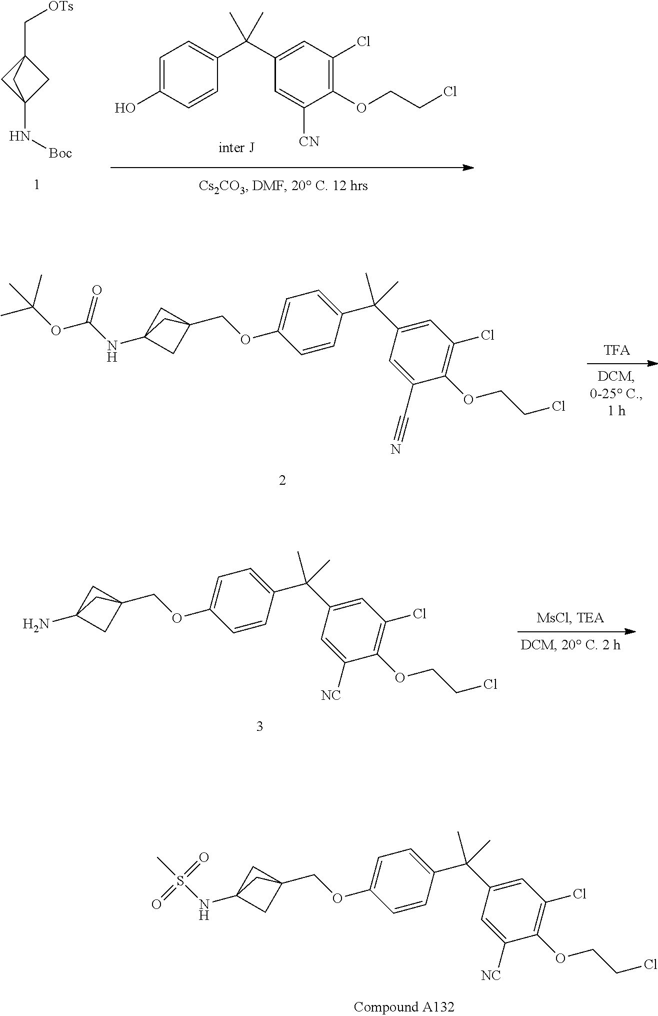 [SMILES: Cc1ccc(S(=O)(=O)OCC23CC(NC(=O)OC(C)(C)C)(C2)C3)cc1 ; CC(C)(c1ccc(O)cc1)c1cc(Cl)c(OCCCl)c(C#N)c1 ; CC(C)(C)OC(=O)NC12CC(COc3ccc(C(C)(C)c4cc(Cl)c(OCCCl)c(C#N)c4)cc3)(C1)C2 ; CC(C)(c1ccc(OCC23CC(N)(C2)C3)cc1)c1cc(Cl)c(OCCCl)c(C#N)c1 ; CC(C)(c1ccc(OCC23CC(NS(C)(=O)=O)(C2)C3)cc1)c1cc(Cl)c(OCCCl)c(C#N)c1]

tert-butyl (3-((4-(2-(3-chloro-4-(2-chloroethoxy)-5-cyanophenyl)propan-2-yl)phenoxy) methyl)bicyclo[1.1.1]pentan-1-yl)carbamate (2): To a mixture of (3-((tert-butoxycarbonyl)amino)bicyclo[1.1.1]pentan-1-yl)methyl 4-methylbenzenesulfonate (1) (210 mg, 0.57 mmol) and 3-chloro-2-(2-chloroethoxy)-5-(2-(4-hydroxyphenyl)propan-2-yl)benzonitrile (inter J) (200 mg, 0.57 mmol) in DMF (5 mL) was added Cs₂CO₃ (372 mg, 1.14 mmol) at 20° C. and the mixture was stirred at 20° C. for 12 hrs. The reaction was quenched with water (10 mL) and extracted with EtOAc (20 mL×3). The combined organic layers were washed with brine (10 mL×2), dried over anhydrous Na₂SO₄, filtered and concentrated under reduced pressure. The residue was purified by silica gel column chromatography to give tert-butyl (3-((4-(2-(3-chloro-4-(2-chloroethoxy)-5-cyanophenyl) propan-2-yl)phenoxy) methyl) bicyclo[1.1.1]pentan-1-yl)carbamate (2) (160 mg, yield: 51.4%) as yellow oil. ¹H-NMR (400 MHz, CDCl₃) δ=7.36-7.35 (m, 1H), 7.25-7.24 (m, 1H), 7.00-6.98 (m, 2H), 6.75-6.72 (m, 2H), 4.34 (t, J=6.0 Hz, 2H), 3.98 (s, 2H), 3.80 (t, J=6.0 Hz, 2H), 1.97 (s, 6H), 1.40-1.35 (m, 9H).

5-(2-(4-((3-aminobicyclo[1.1.1]pentan-1-yl)methoxy)phenyl)propan-2-yl)-3-chloro-2-(2-chloroethoxy)benzonitrile (3): To a solution of tert-butyl (3-((4-(2-(3-chloro-4-(2-chloroethoxy)-5-cyanophenyl)propan-2-yl)phenoxy)methyl)bicyclo[1.1.1]pentan-1-yl) carbamate (2) (160 mg, 0.29 mmol) in DCM (2 mL) was added TFA (0.4 mL) at 0° C. The mixture was stirred at 25° C. for 1 h. LCMS showed the reaction was completed. The mixture was concentrated under reduced pressure to give 5-(2-(4-((3-aminobicyclo[1.1.1]pentan-1-yl)methoxy)phenyl)propan-2-yl)-3-chloro-2-(2-chloroethoxy)benzonitrile (3) (130 mg, yield: 99.5%) as colorless oil. ¹H-NMR (400 MHz, CDCl₃) δ=7.46-7.44 (m, 1H), 7.27 (s, 1H), 7.10-7.04 (m, 2H), 6.82-6.78 (m, 2H), 4.44-4.40 (m, 2H), 4.07 (s, 2H), 3.89-3.86 (m, 2H), 1.63 (s, 6H).

N-(3-((4-(2-(3-chloro-4-(2-chloroethoxy)-5-cyanophenyl)propan-2-yl)phenoxy)methyl) bicyclo[1.1.1]pentan-1-yl)methanesulfonamide (Compound A132): To a solution of 5-(2-(4-((3-aminobicyclo[1.1.1]pentan-1-yl)methoxy)phenyl)propan-2-yl)-3-chloro-2-(2-chloroethoxy) benzonitrile (3) (130 mg, 0.29 mmol) and N,N-diethylethanamine (0.1 mL, 0.58 mmol) in DCM (2 mL) was added methanesulfonyl chloride (33 mg, 0.29 mmol) at 0° C. The mixture was stirred at 20° C. for 2 hrs. LCMS showed the reaction was completed. The resulting mixture was quenched with water (5 mL) and extracted with DCM (3 mL×3). The combined organic layers were washed with brine (3 mL×2), dried over Na₂SO₄, filtered and concentrated under reduced pressure. The residue was purified by p-HPLC (neutral) to give N-(3-((4-(2-(3-chloro-4-(2-chloroethoxy)-5-cyanophenyl)propan-2-yl)phenoxy)methyl)bicyclo[1.1.1]pentan-1-yl) methanesulfonamide (Compound A132) (27.9 mg, yield: 18.3%) as white solid. ¹H-NMR (400 MHz, CDCl₃) δ=1H-NMR (400 MHz, CDCl₃) δ=7.44 (d, J=2.4 Hz, 1H), 7.31 (d, J=2.4 Hz, 1H), 7.08 (d, J=9.2 Hz, 2H), 6.82 (d, J=8.8 Hz, 2H), 4.98 (br s, 1H), 4.42 (t, J=6.0 Hz, 2H), 4.08 (s, 2H), 3.88 (t, J=6.0 Hz, 2H), 3.01 (s, 3H), 2.13 (s, 6H), 1.64 (s, 6H). LCMS (220 nm): 100%. Exact Mass: 522.1; found 540.1/542.1 (M+18).

### Example 22: Synthesis of N-(4-((4-(2-(4-(3-chloro-2-hydroxypropoxy)phenyl)propan-2-yl)phenoxy)methyl)pyrimidin-2-yl)methanesulfonamide (Compound A150)

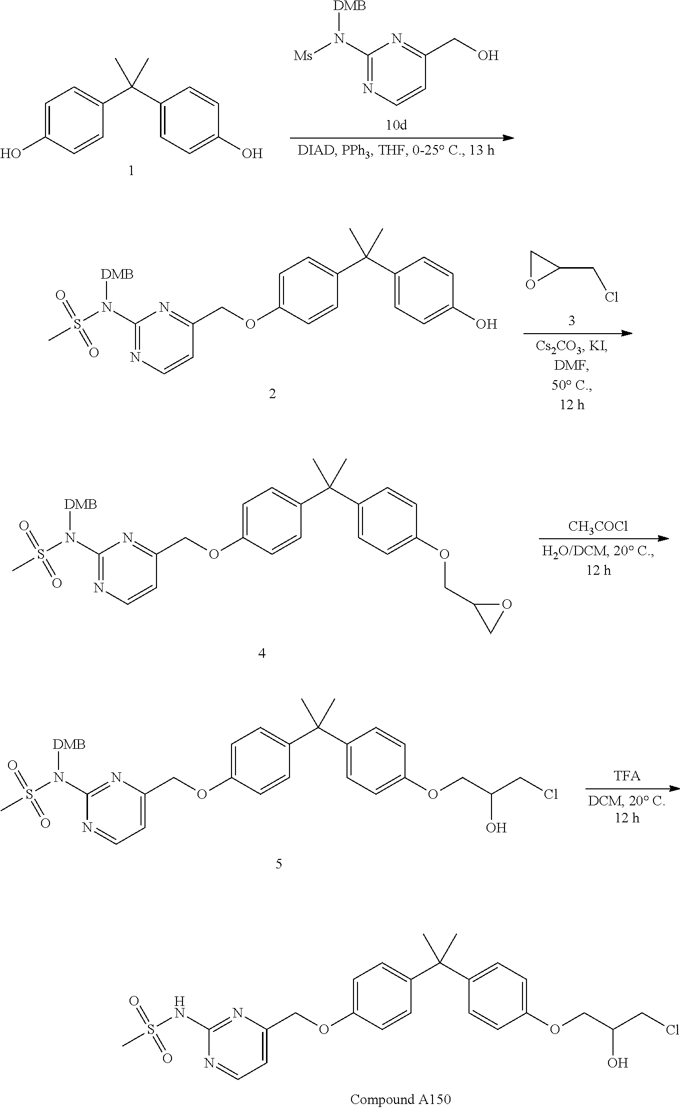

N-(2,4-dimethoxybenzyl)-N-(4-((4-(2-(4-hydroxyphenyl)propan-2-yl)phenoxy)methyl)pyrimidin-2-yl)methanesulfonamide (2): To a solution of 4,4′-(propane-2,2-diyl)diphenol (1) (1.00 g, 4.4 mmol) and N-(2,4-dimethoxybenzyl)-N-(4-(hydroxymethyl)pyrimidin-2-yl)methanesulfonamide (10d) (1.55 g, 4.4 mmol) in THF (13 mL) was added PPh₃ (1.72 g, 6.6 mmol) at 0° C. under N₂. Then DIAD (1.33 g, 6.6 mmol) was added drop-wise at 0° C. under N₂. The mixture was stirred at 25° C. for 13 h. LCMS showed the reaction was completed. The mixture was poured into H₂O (8 mL) extracted with EtOAc (5 mL×3). The organic layers were washed with brine (10 mL), dried over anhydrous Na₂SO₄, filtered and concentrated under reduced pressure. The residue was purified by silica gel column chromatography to give N-(2,4-dimethoxybenzyl)-N-(4-((4-(2-(4-hydroxyphenyl) propan-2-yl) phenoxy)methyl)pyrimidin-2-yl)methanesulfonamide (1.7 g, yield: 34.4%) as yellow oil.

N-(4-((4-(2-(4-(3-chloro-2-hydroxypropoxy)phenyl)propan-2-yl)phenoxy)methyl)pyrimidin-2-yl)-N-(2,4-dimethoxybenzyl)methanesulfonamide (4): To a solution of N-(2,4-dimethoxybenzyl)-N-(4-((4-(2-(4-hydroxyphenyl)propan-2-yl)phenoxy) methyl)pyrimidin-2-yl)methanesulfonamide (2) (100 mg, 0.2 mmol) and dicesium; carbonate (173 mg, 0.5 mmol) in DMF (5 mL) was added potassium; iodide (41 mg, 0.2 mmol) and (2S)-2-(chloromethyl)oxirane (3) (50 mg, 0.5 mmol) at 20° C. The mixture was stirred at 50° C. for 12 h. LCMS showed the reaction was completed. The reaction was quenched with H₂O (10 mL). The aqueous layer was extracted with EtOAc (10 mL×3). The combined organic layers were washed with brine (10 mL), dried over anhydrous Na₂SO₄, filtered and concentrated under reduced pressure. The residue was purified by silica gel column chromatography (PE:EtOAc=1:0 to 7:3) to give N-(2,4-dimethoxybenzyl)-N-(4-((4-(2-(4-(oxiran-2-ylmethoxy)phenyl)propan-2-yl)phenoxy)methyl)pyrimidin-2-yl)methanesulfonamide (1.00 g, yield: 58.6%) as white solid. ¹H-NMR (400 MHz, CDCl₃) δ=8.55 (d, J=4.8 Hz, 1H), 7.25-7.18 (m, 2H), 7.12 (dd, J=2.4, 8.8 Hz, 4H), 6.82 (dd, J=2.0, 8.8 Hz, 4H), 6.49-6.32 (m, 2H), 5.05 (s, 2H), 4.18 (dd, J=3.2, 11.2 Hz, 1H), 3.94 (dd, J=5.6, 11.2 Hz, 1H), 3.76 (s, 3H), 3.61 (s, 3H), 3.33 (dt, J=3.2, 6.0 Hz, 1H), 3.27 (s, 3H), 2.74 (dd, J=2.4, 5.2 Hz, 1H), 1.94 (s, 2H), 1.62 (s, 6H).

N-(4-((4-(2-(4-(3-chloro-2-hydroxypropoxy)phenyl)propan-2-yl)phenoxy)methyl)pyrimidin-2-yl)-N-(2,4-dimethoxybenzyl)methanesulfonamide (5): To a solution of N-(2,4-dimethoxybenzyl)-N-(4-((4-(2-(4-(oxiran-2-ylmethoxy)phenyl)propan-2-yl)phenoxy)methyl)pyrimidin-2-yl)methanesulfonamide (150 mg, 0.24 mmol) in DCM (1 mL) and H₂O (1 mL) was added acetyl chloride (29 mg, 0.36 mmol) at 20° C. The mixture was stirred at 20° C. for 12 h. LCMS showed the reaction was completed. The reaction mixture was diluted with H₂O (10 mL). The resulting aqueous mixture were extracted with DCM (5 mL×3). The combined organic layers were washed with brine (5 mL), dried over anhydrous Na₂SO₄, filtered and concentrated under reduced pressure. The residue purified by p-TLC (SiO2, PE:EtOAc=3:1) to give N-(4-((4-(2-(4-(3-chloro-2-hydroxypropoxy)phenyl)propan-2-yl)phenoxy)methyl)pyrimidin-2-yl)-N-(2,4-dimethoxybenzyl)methanesulfonamide (40 mg, yield: 22.7%) as white solid. ¹H-NMR (400 MHz, CDCl₃) δ=8.57 (d, J=5.2 Hz, 1H), 7.25-7.20 (m, 2H), 7.14 (br dd, J=2.4, 8.8 Hz, 4H), 6.89-6.74 (m, 4H), 6.47-6.29 (m, 2H), 5.06 (s, 2H), 4.21 (td, J=5.2, 10.8 Hz, 1H), 4.11-4.02 (m, 2H), 3.81-3.76 (m, 3H), 3.76-3.68 (m, 2H), 3.62 (s, 3H), 3.28 (s, 3H), 1.64 (s, 6H).

N-(4-((4-(2-(4-(3-chloro-2-hydroxypropoxy)phenyl)propan-2-yl)phenoxy)methyl)pyrimidin-2-yl)methanesulfonamide (Compound A150): A solution of N-(4-((4-(2-(4-(3-chloro-2-hydroxypropoxy)phenyl)propan-2-yl)phenoxy)methyl)pyrimidin-2-yl)-N-(2,4-dimethoxybenzyl)methanesulfonamide (5) (40 mg, 0.01 mmol) in DCM (1 mL) and 2,2,2-trifluoroacetic acid (0.2 mL) was stirred at 20° C. for 12 h. TLC showed the reaction was completed. The mixture was concentrated and then adjusted to pH=8-9 with sat.NaHCO₃. The mixture was extracted with DCM (5 mL×2). The combined organic layers were washed with brine (10 mL), dried over anhydrous Na₂SO₄, filtered and concentrated under reduced pressure. The residue was purified by p-HPLC (FA) to give N-(4-((4-(2-(4-(3-chloro-2-hydroxypropoxy)phenyl)propan-2-yl)phenoxy)methyl)pyrimidin-2-yl)methanesulfonamide (5 mg, yield: 16.2%) as white solid. ¹H-NMR (400 MHz, MeOD) δ=8.53 (d, J=5.2 Hz, 1H), 7.21 (d, J=5.2 Hz, 1H), 7.17-7.10 (m, 4H), 6.91-6.86 (m, 2H), 6.86-6.81 (m, 2H), 5.10 (s, 2H), 4.14-4.07 (m, 1H), 4.02 (dd, J=1.6, 5.2 Hz, 2H), 3.79-3.73 (m, 1H), 3.70-3.63 (m, 1H), 3.36 (s, 3H), 1.61 (s, 6H). LCMS (M+1) m/z: calcd 506.

### Example 23: Synthesis of N-(4-((4-(2-(3-cyano-5-fluoro-4-(2-azaspiro[3.3]heptan-2-yl)phenyl)propan-2-yl)phenoxy)methyl)pyrimidin-2-yl)methanesulfonamide (Compound A740)

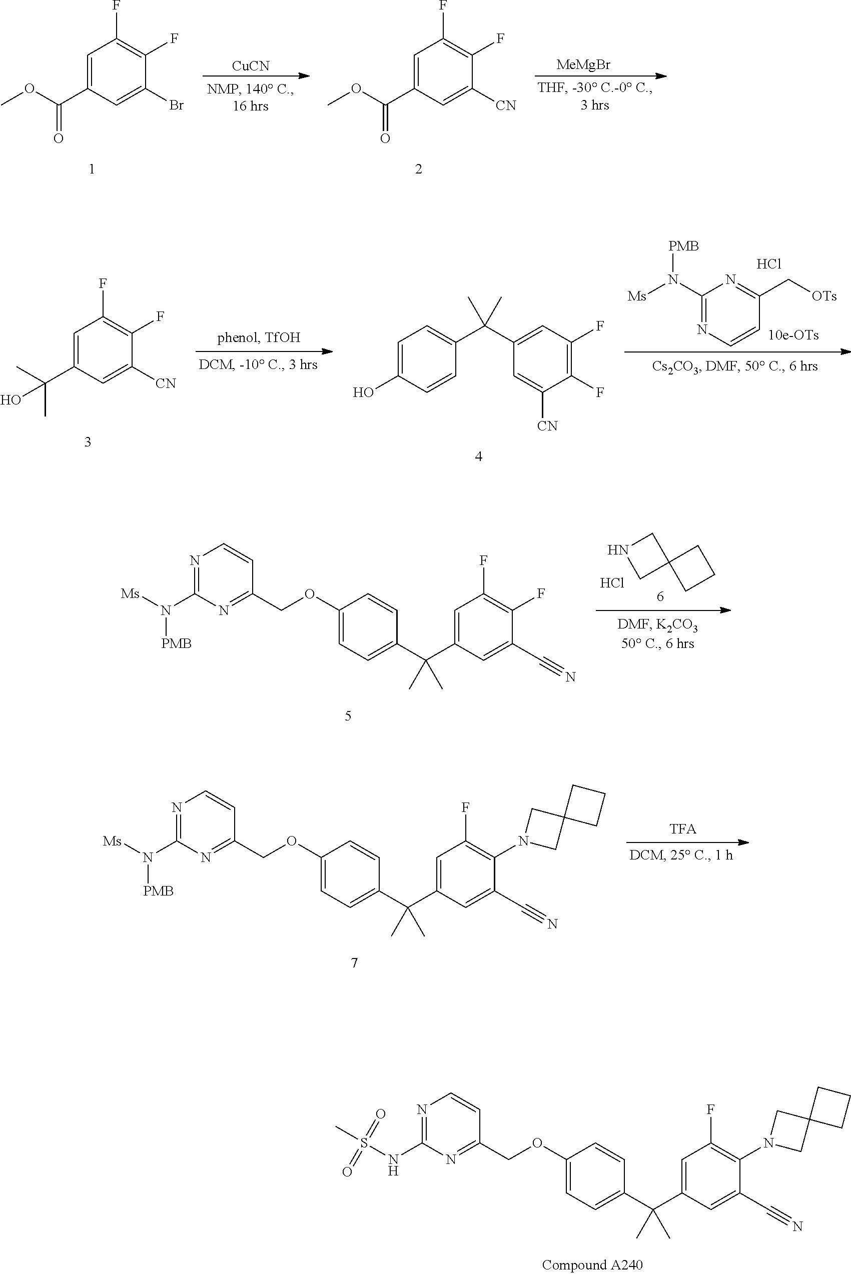

methyl 3-cyano-4,5-difluorobenzoate (2): To a solution of methyl 3-bromo-4,5-difluorobenzoate (1) (20.0 g, 79.7 mmol) in NMP (250 mL) was added CuCN (10.8 g, 0.120 mol) and 4A M.S. (10 g) at 25° C. and the mixture was stirred at 140° C. for 16 hrs under N₂ atmosphere. LCMS showed the reaction was completed. The resulting mixture was filtered and the filtrate was poured into sat. NaCl/NH₃.H₂O (V=5/1, 300 mL) and then extracted with EtOAc (200 mL×3). The combined organic layers were washed with sat.NaCl/NH₃.H₂O (5/1, 100 mL x 2) and brine (100 mL×5), dried over Na₂SO₄, filtered and concentrated under reduced pressure. The residue was purified by silica gel column chromatography to give methyl 3-cyano-4,5-difluorobenzoate (2) (10.2 g, yield: 58.4%) as a white solid. ¹H-NMR (400 MHz, CDCl₃) δ=8.17-8.07 (m, 2H), 3.98 (d, J=1.2 Hz, 3H).

2,3-difluoro-5-(2-hydroxypropan-2-yl)benzonitrile (3): To a solution of MeMgBr (3 mol/L, 38.6 mL, 116 mmol) was added methyl 3-cyano-4,5-difluorobenzoate (2) (8 g, 38.6 mmol) in 50 mL THF dropwise at −30° C. under N₂. The mixture was stirred for 3 hrs at 0° C. under N₂. LCMS showed the reaction was completed. The reaction was quenched with sat.NH₄Cl (200 mL) and then extracted with EtOAc (100 mL×3). The combined organic layers were washed with brine (200 mL), dried over Na₂SO₄, filtered and concentrated under reduced pressure to give 2,3-difluoro-5-(2-hydroxypropan-2-yl)benzonitrile (3) (8.0 g, yield: 94.7%) as yellow oil. ¹H-NMR (400 MHz, CDCl₃) δ=7.60 (ddd, J=2.4, 8.4, 10.4 Hz, 1H), 7.51 (dd, J=2.4, 3.2 Hz, 1H), 1.63-1.56 (m, 6H).

2,3-difluoro-5-(2-(4-hydroxyphenyl)propan-2-yl)benzonitrile (4): To a solution of 2,3-difluoro-5-(2-hydroxypropan-2-yl)benzonitrile (3) (9.0 g, 41.1 mmol) and phenol (11.6 g, 123 mmol) in DCM (100 mL) was added trifluoromethanesulfonic acid (2.7 g, 18.3 mmol) dropwise at −10° C. The mixture was stirred at −10° C. for 3 hrs under N₂ atmosphere. LCMS showed the reaction was completed. The reaction was quenched with sat.NH₄Cl (200 mL) and then extracted with DCM (100 mL×3). The combined organic layers were washed with brine (100 mL), dried over Na₂SO₄, filtered and concentrated under reduced pressure. The residue was triturated by isopropyl ether (50 mL) to give 2,3-difluoro-5-(2-(4-hydroxyphenyl)propan-2-yl)benzonitrile (4) (8.0 g, yield: 67.7%) as off-white solid. ¹H-NMR (400 MHz, CDCl₃) δ=7.27 (s, 2H), 7.05 (d, J=8.4 Hz, 2H), 6.79 (d, J=8.4 Hz, 2H), 4.79 (br s, 1H), 1.65 (s, 6H).

N-(4-((4-(2-(3-cyano-4,5-difluorophenyl)propan-2-yl)phenoxy)methyl)pyrimidin-2-yl)-N-(4-methoxybenzyl)methanesulfonamide (5): To a solution of (2-(N-(4-methoxybenzyl)methylsulfonamido)pyrimidin-4-yl)methyl 4-methylbenzene sulfonate (10e-OTs) (900 mg, 1.79 mmol) and 2,3-difluoro-5-(2-(4-hydroxyphenyl)propan-2-yl)benzonitrile (4) (412 mg, 1.43 mmol) in DMF (9 mL) was added dicesium; carbonate (1.75 g, 5.37 mmol) and tetrabutylammonium; iodide (132 mg, 0.36 mmol) at 20° C. The reaction was stirred at 50° C. for 6 h. LCMS showed the starting material was consumed and desired was detected. The reaction was quenched with H₂O (10 mL) and then extracted with EtOAc (10 mL×3). The combined organic layers were washed with brine (20 mL), dried over anhydrous Na₂SO₄, filtered and concentrated under reduced pressure. The residue was purified by silica gel column chromatography to give N-(4-((4-(2-(3-cyano-4,5-difluorophenyl)propan-2-yl)phenoxy)methyl)pyrimidin-2-yl)-N-(4-methoxybenzyl)methanesulfonamide (5) (450 mg, yield: 17.4%) as colorless oil. ¹H-NMR (400 MHz, CDCl₃) δ=8.60 (d, J=5.2 Hz, 1H), 7.37 (d, J=8.4 Hz, 2H), 7.24-7.20 (m, 2H), 7.17-7.08 (m, 3H), 6.93-6.86 (m, 3H), 6.83 (d, J=8.4 Hz, 2H), 5.36 (s, 2H), 5.13-5.05 (m, 2H), 3.78 (s, 3H), 3.41-3.36 (m, 3H), 1.65 (s, 5H).

N-(4-((4-(2-(3-cyano-5-fluoro-4-(2-azaspiro[3.3]heptan-2-yl)phenyl)propan-2-yl)phenoxy)methyl)pyrimidin-2-yl)-N-(4-methoxybenzyl)methanesulfonamide (7): To a solution of N-(4-((4-(2-(3-cyano-4,5-difluorophenyl)propan-2-yl) phenoxy)methyl)pyrimidin-2-yl)-N-(4-methoxybenzyl)methanesulfonamide (5) (30 mg, 0.05 mmol) and 2-azaspiro[3.3]heptane hydrochloride (6) (7 mg, 0.05 mmol) in DMF (0.5 mL) was added dipotassium; carbonate (22 mg, 0.16 mmol) at 20° C. and the mixture was stirred at 50° C. for 6 hrs. LCMS showed the reaction was completed. The mixture was quenched with water (3 mL) and extracted with EtOAc (2 mL×3). The combined organic layers were washed with brine (2 mL×2), dried over anhydrous Na₂SO₄, filtered and concentrated under reduce pressure. The residue was purified by p-TLC to give N-(4-((4-(2-(3-cyano-5-fluoro-4-(2-azaspiro[3.3]heptan-2-yl)phenyl)propan-2-yl)phenoxy)methyl)pyrimidin-2-yl)-N-(4-methoxybenzyl) methanesulfonamide (7) (50 mg, yield: 88.2%) as yellow oil. ¹H-NMR (400 MHz, CDCl₃) δ=8.59 (d, J=5.2 Hz, 1H), 7.37 (d, J=8.6 Hz, 2H), 7.22 (d, J=5.2 Hz, 1H), 7.12-7.08 (m, 2H), 7.02 (d, J=1.2 Hz, 1H), 6.88-6.79 (m, 5H), 5.36 (s, 2H), 5.08 (s, 2H), 3.78 (s, 3H), 3.38 (s, 3H), 2.20 (t, J=7.6 Hz, 4H), 1.85 (t, J=7.6 Hz, 2H), 1.56 (d, J=7.2 Hz, 10H).

N-(4-((4-(2-(3-cyano-5-fluoro-4-(2-azaspiro[3.3]heptan-2-yl)phenyl)propan-2-yl)phenoxy)methyl)pyrimidin-2-yl)methanesulfonamide (Compound A240): To a solution of N-(4-((4-(2-(3-cyano-5-fluoro-4-(2-azaspiro[3.3]heptan-2-yl)phenyl) propan-2-yl)phenoxy)methyl)pyrimidin-2-yl)-N-(4-methoxybenzyl) methanesulfonamide (7) (60 mg, 0.08 mmol) in DCM (1 mL) was added TFA (0.1 mL) at 25° C. and the mixture was stirred at 25° C. for 1 h. LCMS showed the reaction was completed. The reaction was poured into sat.NaHCO₃ to adjust pH=7-8 and then extracted with DCM (5 mL×3). The combined organic layers were washed with brine (5 mL), dried over Na₂SO₄, filtered and concentrated under reduced pressure. The residue was purified by p-TLC to give N-(4-((4-(2-(3-cyano-5-fluoro-4-(2-azaspiro[3.3]heptan-2-yl)phenyl)propan-2-yl)phenoxy)methyl)pyrimidin-2-yl) methanesulfonamide (Compound A240) (9.4 mg, yield: 20.7%) as off-white solid. ¹H-NMR (400 MHz, CDCl₃) δ=9.50 (br s, 1H), 8.66 (br d, J=3.6 Hz, 1H), 7.29 (br s, 1H), 7.12 (br d, J=8.4 Hz, 2H), 7.02 (s, 1H), 6.94-6.75 (m, 3H), 5.11 (s, 2H), 4.33 (br d, J=2.0 Hz, 4H), 3.47 (s, 3H), 2.20 (br t, J=7.6 Hz, 4H), 1.88-1.80 (m, 2H), 1.58 (s, 6H). LCMS (220 nm): 97.1%. Exact Mass: 535.2; found 536.2.

### Example 24: Synthesis of 3-chloro-2-(2-chloroethoxy)-5-(5-((6-(methylsulfonyl)-2,6-diazaspiro[3.3] heptan-2-yl)methyl)-1H-indol-1-yl)benzonitrile (Compound A241)

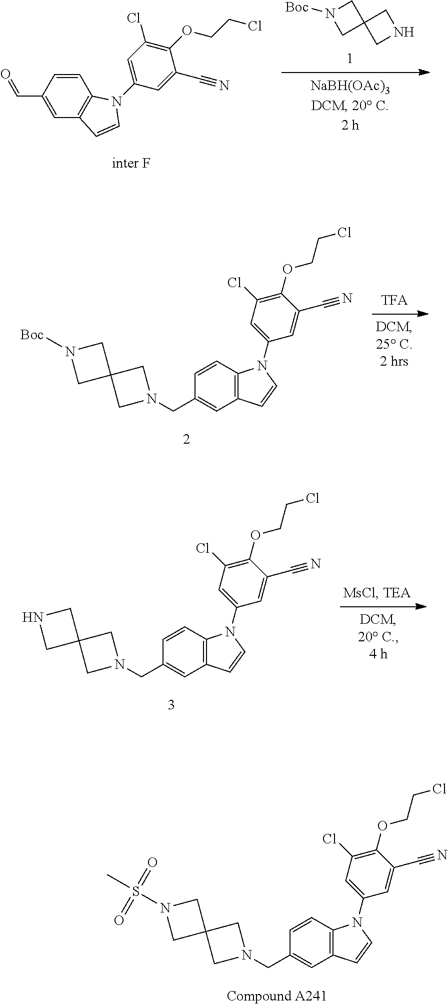 [SMILES: N#Cc1cc(-n2ccc3cc(C=O)ccc32)cc(Cl)c1OCCCl ; CC(C)(C)OC(=O)N1CC2(CNC2)C1 ; CC(C)(C)OC(=O)N1CC2(CN(Cc3ccc4c(ccn4-c4cc(Cl)c(OCCCl)c(C#N)c4)c3)C2)C1 ; N#Cc1cc(-n2ccc3cc(CN4CC5(CNC5)C4)ccc32)cc(Cl)c1OCCCl ; CS(=O)(=O)N1CC2(CN(Cc3ccc4c(ccn4-c4cc(Cl)c(OCCCl)c(C#N)c4)c3)C2)C1]

Tert-butyl 6-((1-(3-chloro-4-(2-chloroethoxy)-5-cyanophenyl)-1H-indol-5-yl) methyl)-2,6-diazaspiro[3.3]heptane-2-carboxylate (2): To a solution of 3-chloro-2-(2-chloroethoxy)-5-(5-formyl-1H-indol-1-yl)benzonitrile (inter F) (300 mg, 0.84 mmol), tert-butyl 2,6-diazaspiro[3.3]heptane-2-carboxylate (1) (182 mg, 0.92 mmol) and 4A M. S. (300 mg) in DCM (5 mL) was added NaBH(OAc)₃ (266 mg, 1.25 mmol) at 20° C. and the mixture was stirred at 20° C. for 2 hrs under N₂ atmosphere. LCMS showed the reaction was completed. The reaction was quenched with H₂O (5 mL) and extracted with DCM (5 mL×3). The combined organic layers were washed with brine (5 mL), dried over anhydrous Na₂SO₄, filtered and concentrated under reduced pressure. The residue was purified by silica gel column chromatography to give tert-butyl 6-((1-(3-chloro-4-(2-chloroethoxy)-5-cyanophenyl)-1H-indol-5-yl)methyl)-2,6-diazaspiro [3.3]heptane-2-carboxylate (2) (240 mg, yield: 47.8%) as colorless oil. ¹H-NMR (400 MHz, CDCl₃) δ=7.79 (d, J=2.4 Hz, 1H), 7.65 (d, J=2.8 Hz, 1H), 7.56 (s, 1H), 7.43 (d, J=8.4 Hz, 1H), 7.24 (d, J=3.2 Hz, 1H), 7.20 (d, J=1.2, 1H), 6.70 (d, J=3.2 Hz, 1H), 4.53 (t, J=6.0 Hz, 2H), 4.00 (s, 4H), 3.95 (t, J=6.0 Hz, 2H), 3.69 (s, 2H), 3.37 (s, 4H), 1.43 (s, 9H).

5-(5-(2,6-diazaspiro[3.3]heptan-2-ylmethyl)-1H-indol-1-yl)-3-chloro-2-(2-chloroethoxy)benzonitrile(3): To a solution of tert-butyl 6-((1-(3-chloro-4-(2-chloroethoxy)-5-cyanophenyl)-1H-indol-5-yl)methyl)-2,6-diazaspiro[3.3]heptane-2-carboxylate (2) (240 mg, 0.40 mmol) in DCM (2 mL) was added TFA (1.5 mL) at 20° C. The mixture was stirred at 20° C. for 1 h. LCMS showed the desired product was detected. The reaction was concentrated under reduced pressure to give 5-(5-(2,6-diazaspiro[3.3]heptan-2-ylmethyl)-1H-indol-1-yl)-3-chloro-2-(2-chloroethoxy) benzonitrile (3) (240 mg, yield: 95.4%) as yellow solid and used directly.

3-chloro-2-(2-chloroethoxy)-5-(5-((6-(methylsulfonyl)-2,6-diazaspiro[3.3]heptan-2-yl)methyl)-1H-indol-1-yl)benzonitrile (Compound A241): To a mixture of 5-(5-(2,6-diazaspiro[3.3]heptan-2-ylmethyl)-1H-indol-1-yl)-3-chloro-2-(2-chloroethoxy) benzonitrile (3) (240 mg, 0.22 mmol) in THF (2 mL) was added NaHCO₃ (1 mL) to adjust pH=8-9, and then added methanesulfonyl chloride (25 mg, 0.22 mmol) at 0° C. The mixture was stirred at 20° C. for 4 hrs. LCMS showed the reaction was completed. The reaction was quenched with water (5 mL) and extracted with DCM (5 mL×3). The combined organic layers were washed with brine (5×3 mL), dried over anhydrous Na₂SO₄, filtered and concentrated under reduced pressure. The residue was purified by p-HPLC (FA) to give 3-chloro-2-(2-chloroethoxy)-5-(5-((6-(methylsulfonyl)-2,6-diazaspiro[3.3]heptan-2-yl)methyl)-1H-indol-1-yl)benzonitrile (Compound A241) (39.3 mg, yield: 48.6%) as white gum. ¹H-NMR (400 MHz, MeOD) δ=8.47 (s, 1H), 7.99 (d, J=2.8 Hz, 1H), 7.89 (d, J=2.4 Hz, 1H), 7.67 (s, 1H), 7.58 (d, J=8.4 Hz, 1H), 7.55 (d, J=3.2 Hz, 1H), 7.26 (d, J=7.6 Hz, 1H), 6.75 (d, J=3.2 Hz, 1H), 4.56 (t, J=5.2 Hz, 2H), 4.05 (s, 6H), 3.98 (t, J=5.2 Hz, 2H), 3.85 (s, 4H), 2.91 (s, 3H). LCMS: (220 nm): 98%. Exact Mass: 518.1; found 519.0/521.0.

### Example 25: Synthesis of Compounds of Table A

Compounds of Table A were synthesized according to modified procedures based on the examples provided herein and/or with organic chemistry reactions known to one skilled in the art. The characterization of the synthesized compounds is provided in Table 1.

TABLE 1
Characterization of Compounds of Table A (M + H by LC-MS)

| Comp | | M + H | M + H |
|---|---|---|---|
| ID | ¹H-NMR | (calc) | (found) |
| A1 | ¹H-NMR (400 MHz, DMSO-d₆) δ = 8.19 (s, 1H), 7.86 (s, 1H), 7.66-7.58 (m, 5H), 7.50-7.31 (m, | 567.1 | 567.0 |
| | 5H), 7.16 (s, 1H), 6.75 (s, 2H), 6.61 (s, 1H), 2.52 (s, 3H), 1.68 (s, 6H) | | |
| A2 | ¹H-NMR (400 MHz, DMSO-d₆) δ = 8.18 (d, J = 5.2 Hz, 1H), 7.85 (d, J = 1.2 Hz, 2H), 7.64 (d, J = 8.4 | 567.1 | 567.0 |
| | Hz, 2H), 7.56 (d, J = 8.4 Hz, 2H), 7.46 (d, J = 8.4 Hz, 2H), 7.15 (s. 1H), 6.75 (s. 2H), 6.61 (d, J = 5.2 | | |
| | Hz, 1H), 2.81(s, 3H), 1.71(s, 6H) | | |
| A5 | ¹H NMR (400 MHz, CDCl₃) δ = ppm 9.35 (br s, 1 H) 8.61 (d, J = 5.2 Hz, 1 H) 7.54 (d, J = 2.0 Hz, | 445.0 | 445.1 |
| | 1 H) 7.42 (d, J = 2.0 Hz, 1 H) 7.05 (d, J = 5.2 Hz, 1 H) 4.56 (s, 2 H) 4.44 (t, J = 6.0 Hz, 2 H) 3.90 (t, | | |
| | J = 6.0 Hz, 2 H) 3.79 (t, J = 6.0 Hz, 2 H) 3.45 (s, 3 H) 2.94 (t, J = 6.0 Hz, 2 H) | | |
| A6 | ¹H NMR (400 MHz, CDCl₃) δ = 8.23 (d, J = 6.8 Hz, 1H), 7.71 (d, J = 1.6 Hz, 1H), 7.57 (d, J = 1.2 | 514.1 | 514.1 |
| | Hz, 1H), 6.31 (d, J = 6.4 Hz, 1H), 5.42 (s, 1H), 4.53 (t, J = 6.0 Hz, 2H), 3.90 (t, J = 6.0 Hz, 2H), 3.71- | | |
| | 3.45 (m, 5H), 3.28 (s, 3H), 2.1-1.95 (m, 4H) | | |
| A7 | ¹H NMR (400 MHz, CDCl₃) δ = 8.21 (d, J = 6.8 Hz, 1H), 8.01 (d, J = 9.2 Hz, 2H), 6.96 (d, J = 8.8 | 452.1 | 452.1 |
| | Hz, 2H), 6.31 (d, J = 6.8 Hz, 1H), 4.52 (s, 2H), 4.30 (t, J = 6.0 Hz, 2H), 3.85 (t, J = 5.6 Hz, 2H), 3.31 | | |
| | (s, 3H), 2.33 (s, 6H) | | |
| A8 | ¹H NMR (400 MHz, CDCl₃) δ = 8.24-8.21 (m, 2H), 8.13(d, J = 2.0 Hz, 1H), 6.35(d, J = 6.4 Hz, | 511.1 | 511.1 |
| | 1H), 4.60 (t, J = 5.6 Hz, 1H), 4.53(s, 2H), 3.91 (t, J = 5.6 Hz, 2H), 3.32 (s, 3H), 2.36 (s, 6H) | | |
| A10 | ¹H-NMR (400 MHz, CDCl₃) δ = 8.01 (s, 1H), 7.78 (d, J = 2.4 Hz, 1H), 7.63 (s, 1H), 7.40 (d, J = 9.2 | 547.1 | 547.0 |
| | Hz, 1H), 7.27 (s, 1H), 7.19 (s, 1H), 6.92 (d, J = 9.2 Hz, 1H), 6.68 (d, J = 3.2 Hz, 1H), 6.14 (d, J = 5.6 | | |
| | Hz, 2H), 4.96 (s, 2H), 4.54 (t, J = 6.0 Hz, 2H), 3.95 (t, J = 5.6 Hz, 2H), 3.25 (s, 3H) | | |
| A18 | ¹H-NMR (400 MHz, DMSO-d₆) δ = 9.26 (s, 1H), 8.66 (d, J = 4.8 Hz, 1H), 8.22 (dd, J = 2.0, 2.0 Hz, | 545.0 | 545.2 |
| | 2H), 8.06 (d, J = 10.0 Hz, 1H), 7.54 (d, J = 2.4 Hz, 2H), 7.30 (d, J = 4.8 Hz, 1H), 5.45 (s, 2H), 4.60 (t, | | |
| | J = 5.2 Hz, 2H), 4.04 (t, J = 5.2 Hz, 2H), 3.37(s, 3H) | | |
| A20 | ¹H-NMR (400 MHz, DMSO-d₆) δ = 7.78 (d, J = 2.4 Hz, 1H), 7.64 (d, J = 2.8 Hz, 2H), 7.41 (d, J = 8.8 | 506.0 | 506.0 |
| | Hz, 1H), 7.25 (d, J = 3.2 Hz, 1H), 7.18 (d, J = 2.4 Hz, 1H), 7.16 (s, 1H), 6.97 (d, J = 3.2 Hz, 1H), | | |
| | 6.66 (d, J = 3.2 Hz, 1H), 5.01 (s, 2H), 4.95 (br s, 2H), 4.54 (t, J = 6.0 Hz, 2H), 3.95 (t, J = 6.0 Hz, 2H) | | |
| A21 | ¹H-NMR (400 MHz, DMSO-d₆) δ = 8.61 (d, J = 5.2 Hz, 1H), 8.43 (d, J = 2.8 Hz, 1H), 8.15 (d, J = 2.4 | 566.1 | 565.9 |
| | Hz, 1H), 7.75 (d, J = 3.2 Hz, 1H), 7.57 (d, J = 9.2 Hz, 1H), 7.26-7.02(m, 2H), 7.00 (d, J = 2.0 Hz, | | |
| | 1H), 6.67 (d, J = 3.2 Hz, 1H), 5.13 (s, 2H), 4.59 (t, J = 5.2 Hz, 2H), 4.03 (t, J = 5.2 Hz, 2H), 5.13 (s, | | |
| | 3H) | | |
| A22 | ¹H-NMR (400 MHz, CDCl₃) δ = 8.62 (d, J = 5.2 Hz, 1H), 7.81 (d, J = 2.4 Hz, 1H), 7.68 (d, J = 2.4 Hz, | 600.0 | 601.9 |
| | 1H), 7.33 (d, J = 4.8 Hz, 1H), 7.26 (d, J = 3.6 Hz, 1H), 7.17 (s, 1 H), 7.16 (d, J = 2.4 Hz, 1H) 7.03 (d, | | |
| | J = 9.2 Hz, 1H), 6.67 (d, J = 3.6 Hz, 1H), 5.19 (s, 2H), 4.89 (s, 2H), 3.49 (s, 3H) | | |
| A25 | ¹H-NMR (400 MHz, DMSO-d₆) δ = 11.42 (s, 1H), 8.60 (d, J = 5.2 Hz, 1H), 8.26 (t, J = 2.0 Hz, 2H), | 507.1 | 507.0 |
| | 8.04 (d, J = 8.8 Hz, 1H), 7.91 (d, J = 1.6 Hz, 1H), 7.77 (d, J = 2.0 Hz, 1H), 7.19 (m, 3H), 6.95 (d, | | |
| | J = 8.8 Hz, 2H), 5.11 (s, 2H), 3.32 (s, 3H), 1.73 (s, 6H) | | |
| A26 | ¹H-NMR (400 MHz, CDCl₃) δ = 8.68 (d, J = 5.2 Hz, 1H), 7.57 (s, 1H), 7.50 (d, J = 3.2 Hz, 1H), | 532.1 | 532.0 |
| | 7.43-7.35 (m, 2H), 7.33 (br d, J = 5.2 Hz, 1H), 7.15 (br d, J = 8.8 Hz, 2H), 6.82 (d, J = 3.2 Hz, 1H), | | |
| | 5.20 (s, 2H), 4.50-4.37 (m, 2H), 3.95 (t, J = 6.4 Hz, 2H), 3.50 (s, 3H). | | |
| A27 | ¹H-NMR (400 MHz, CDCl₃) δ = 8.76-8.55 (m, 1H), 7.61 (s, 1H), 7.42 (d, J = 3.2 Hz, 1H), 7.37 (br | 532.1 | 532.0 |
| | d, J = 8.4 Hz, 2H), 7.32-7.28 (m, 1H), 7.13 (br d, J = 8.4 Hz, 2H), 6.79 (d, J = 3.2 Hz, 1H), 5.17 (br s, | | |
| | 2H), 4.44 (t, J = 6.0 Hz, 2H), 3.94 (t, J = 6.0 Hz, 2H), 3.47 (br s, 3H). | | |
| A28 | ¹H-NMR (400 MHz, DMSO-d₆) δ = 11.42 (s, 1H), 8.61(d, J = 5.2 Hz, 1H), 7.56(d, J = 3.2 Hz, 1H), | 550.1 | 550.0 |
| | 7.52(d, J = 2.8 Hz, 1H), 7.18(d, J = 5.2 Hz, 1H), 6.95(d, J = 8.8 Hz, 1H), 6.59(d, J = 2.8 Hz, 1H), | | |
| | 6.54(dd, J = 2.8, 8.8 Hz, 1H), 5.08(s, 2H), 4.35(t, J = 4.8 Hz, 2H), 4.17(t, J = 3.6 Hz, 2H), 3.95(t, | | |
| | J = 5.2 Hz, 2H), 3.69(t, J = 4.0 Hz, 2H), 3.32(s, 3H) | | |
| A29 | ¹H-NMR (400 MHz, CDCl₃) δ = 8.72 (s, 2H), 7.78 (d, J = 2.4 Hz, 1H), 7.64 (d, J = 2.4 Hz, 1H), 7.40 | 532.1 | 532.0 |
| | (d, J = 5.6 Hz, 1H), 7.26 (d, J = 3.6 Hz, 1H), 7.21 (d, J = 2.4 Hz, 1H), 6.99-6.95 (m, 1H), 6.67 (d, | | |
| | J = 3.2 Hz, 1H), 5.09 (s, 2H), 4.64 (t, J = 6.0 Hz, 1H), 3.95 (t, J = 6.0 Hz, 1H), 3.49 (s, 3H) | | |
| A30 | ¹H-NMR (400 MHz, DMSO-d₆) δ = 11.50-11.31 (m, 1H), 8.62 (d, J = 5.2 Hz, 1H), 7.52 (dd, J = 2.8, | 534.1 | 534.0 |
| | 14.4 Hz, 2H), 7.18 (br d, J = 5.2 Hz, 1H), 7.13 (d, J = 8.8 Hz, 1H), 7.00 (d, J = 1.6 Hz, 1H), 6.76 (dd, | | |
| | J = 2.4, 8.4 Hz, 1H), 5.09 (s, 2H), 4.35-4.31 (m, 2H), 3.98-3.91 (m, 4H), 3.36 (s, 3H), 3.08 (br t, | | |
| | J = 8.4 Hz, 2H). | | |
| A31 | ¹H-NMR (400 MHz, CDCl₃) δ = 8.63 (d, J = 5.2 Hz, 1H), 7.42 (d, J = 2.0 Hz, 1H), 7.30 (d, J = 5.2 | 533.1 | 533.0 |
| | Hz, 1H), 7.28 (d, J = 2.0 Hz, 1H), 6.91 (s, 1H), 6.86 (d, J = 8.4 Hz, 1H), 6.80 (d, J = 2.0 Hz, 1H), | | |
| | 5.12 (s, 2H), 4.44 (d, J = 6.4 Hz, 2H), 4.28 (d, J = 6.0 Hz, 1H), 3.89 (d, J = 6.4 Hz, 2H), 3.49 (s, 3H), | | |
| | 3.03-2.94 (m, 2H), 2.65-2.61 (m, 1H), 2.03-1.95 (m, 1H) | | |
| A32 | ¹H-NMR (400 MHz, CDCl₃) δ = 8.63 (d, J = 5.2 Hz, 1H), 7.60 (d, J = 2.4 Hz, 1H), 7.48 (d, J = 2.4 | 545.1 | 545.1 |
| | Hz, 1H), 7.28 (s, 1H), 6.86 (d, J = 2.8 Hz, 1H), 6.83 (d, J = 8.8 Hz, 1H), 6.73-6.70 (m, 1H), 6.02 (t, | | |
| | J = 4.8 Hz, 1H), 5.12 (s, 2H), 5.50 (t, J = 6.0 Hz, 2H), 3.93 (t, J = 6.0 Hz, 2H), 3.48 (s, 3H), 2.85-2.11 | | |
| | (m, 2H), 2.44-2.41 (m, 2H) | | |
| A33 | ¹H-NMR (400 MHz, CDCl₃) δ = 8.64 (d, J = 5.2 Hz, 1H), 7.36 (d, J = 2.0 Hz, 1H), 7.35-7.29 (m, | 547.1 | 547.0 |
| | 1H), 7.17 (d, J = 2.0 Hz, 1H), 6.76 (s, 1H), 6.72 (s, 2H), 5.10 (s, 2H), 4.43 (t, J = 6.0 Hz, 2H), 4.06 | | |
| | (t, J = 5.2 Hz, 1H), 3.89 (t, J = 6.4 Hz, 2H), 3.48 (s, 3H), 2.92-2.78 (m, 2H), 2.18-2.12 (m, 1H), | | |
| | 1.82-1.56 (m, 3H) | | |
| A34 | ¹H-NMR (400 MHz, DMSO-d₆) δ = 8.61 (d, J = 5.2 Hz, 1H), 7.97-7.73 (m, 3H), 7.72 (d, J = 9.2 Hz, | 543.1 | 543.0 |
| | 1H), 7.57-7.56 (m, 2H), 7.36-7.34 (m, 2H), 7.20 (s, 1H), 5.28 (s, 2H), 4.56 (t, J = 5.2 Hz, 2H), 4.05 | | |
| | (t, J = 5.2 Hz), 3.31 (s, 3H). | | |
| A35 | ¹H-NMR (400 MHz, CDCl₃) δ = 8.48-8.32 (m, 1H), 7.39 (s, 1H), 7.23 (br s, 1H), 6.92-6.65 (m, | 533.1 | 533.0 |
| | 4H), 5.07-4.78 (m, 2H), 4.42 (t, J = 6.0 Hz, 2H), 4.27-4.17 (m, 1H), 3.87 (t, J = 6.0 Hz, 2H), 3.37- | | |
| | 3.10 (m, 3H), 2.98-2.84 (m, 2H), 2.61-2.51 (m, 1H), 1.94-1.88 (m, 1H). | | |
| A36 | ¹H-NMR (400 MHz, DMSO-d₆) δ = 8.62 (d, J = 5.2 Hz, 1H), 8.11 (dd, J = 2.8, 11.2 Hz, 2H), 7.69 | 532.1 | 531.8 |
| | (d, J = 3.2 Hz, 1H), 7.57 (d, J = 8.8 Hz, 1H), 7.31-7.18 (m, 2H), 7.00 (dd, J = 2.4, 9.2 Hz, 1H), 6.65 | | |
| | (d, J = 3.2 Hz, 1H), 5.18 (s, 2H), 4.56-4.43 (m, 2H), 4.11-3.97 (m, 2H), 3.38 (s, 3H) | | |
| A37 | ¹H NMR (400 MHz, CDCl₃) δ = 8.61 (d, J = 5.2 Hz, 1H), 8.07 (d, J = 2.0 Hz, 1H), 7.97 (d, J = | 485.0 | 485.0 |
| | 2.0 Hz, 1H), 7.01 (d, J = 5.2 Hz, 1H), 5.06 (s, 2H), 4.57 (t, J = 6.0 Hz, 2H), 3.91 (t, J = 6.0 Hz, | | |
| | 2H), 3.39 (s, 3H) | | |
| A38 | ¹H NMR (400 MHz, CDCl₃) δ = 8.60 (d, J = 5.2 Hz, 1H), 7.29 (d, J = 4.8 Hz, 1H), 7.16 (d, J = 8.8 | 490.1 | 490.1 |
| | Hz, 2H), 6.86 (d, J = 8.8 Hz, 2H), 6.79 (d, J = 2.0 Hz, 1H), 6.64 (d, J = 2.4 Hz, 1H), 5.08 (s, 2H), | | |
| | 4.33 (t, J = 3.2 Hz, 2H), 4.25 (t, J = 1.6 Hz, 2H), 3.46 (s, 3H), 1.60 (s, 6H) | | |
| A40 | ¹H-NMR (400 MHz, CDCl₃) δ = 8.61 (d, J = 4.8 Hz, 1H), 8.14 (d, J = 2.8 Hz, 1H), 7.96 (d, J = 2.4 | 534.0 | 533.9 |
| | Hz, 1H), 7.68 (br d, J = 9.2 Hz, 1H), 7.51 (d, J = 2.4 Hz, 1H), 7.44 (d, J = 2.4 Hz, 1H), 7.29 (d, J = 5.2 | | |
| | Hz, 1H), 5.22 (s, 2H), 4.60 (t, J = 6.4 Hz, 2H), 3.96 (t, J = 6.0 Hz, 2H), 3.51 (s, 3H) | | |
| A41 | ¹H-NMR (400 MHz, CDCl₃) δ = 8.59 (d, J = 5.2 Hz, 1H), 8.30 (s, 1H), 7.93 (d, J = 2.0 Hz, 1H), 7.80 | 501.0 | 500.8 |
| | (d, J = 2.0 Hz, 1H), 6.96 (d, J = 5.2 Hz, 1H), 5.24 (s, 2H), 4.55 (d, J = 6.0 Hz, 2H), 3.91 (d, J = 6.0 Hz, | | |
| | 2H), 3.41 (s, 3H) | | |
| A43 | ¹H-NMR (400 MHz, CDCl₃) δ = 7.80 (s, 1H), 7.44 (d, J = 2.0 Hz, 1H), 7.30 (d, J = 2.4 Hz, 1H), 7.10 | 550.1 | 549.9 |
| | (d, J = 8.8 Hz, 2H), 6.94 (d, J = 3.2 Hz, 2H), 5.20 (s, 4H), 4.42 (t, J = 6.0 Hz, 2H), 3.88 (t, J = 6.0 Hz, | | |
| | 2H), 3.28 (s, 3H), 1.63 (s, 6H) | | |
| A44 | ¹H-NMR (400 MHz, CDCl₃) δ = 8.41-8.38 (m, 1H), 7.38 (s, 1H), 7.23 (s, 1H), 6.84-6.70 (m, 4H), | 533.1 | 533.0 |
| | 4.95-4.90 (m, 2H), 4.42 (t, J = 3.2 Hz, 2H), 4.21 (d, J = 7.6 Hz, 1H), 3.87 (t, J = 6.0 Hz, 2H), 3.27- | | |
| | 3.21 (m, 3H), 2.92-2.88 (m, 2H), 2.56(br s, 1H), 1.94-1.82 (m, 1H) | | |
| A45 | ¹H-NMR (400 MHz, CDCl₃) δ = 8.66-8.42 (m, 1H), 7.42 (d, J = 2.0 Hz, 1H), 7.28 (s, 1H), 7.26 (s, | 533.1 | 533.0 |
| | 1H), 6.90-6.77 (m, 3H), 5.10 (s, 2H), 4.43 (t, J = 6.0 Hz, 2H), 4.29-4.25 (m, 1H), 3.89 (t, J = 6.0 Hz, | | |
| | 2H), 3.46 (br, 3H), 3.01-2.93 (m, 2H), 2.63-2.59 (m, 2H), 2.00-1.94 (m, 1H) | | |
| A46 | ¹H-NMR (400 MHz, DMSO-d₆) δ = 11.41 (br s, 1H), 8.65 (s, 2H), 8.30 (d, J = 2.4 Hz, 1H), 8.25 (d, | 567.0 | 566.9 |
| | J = 2.4 Hz, 1H)), 7.61 (d, J = 9.2 Hz, 1H), 7.32 (d, J = 9.2 Hz, 1H), 7.28 (d, J = 5.2 Hz, 1H), 5.30 (s, | | |
| | 2H), 4.54 (t, J = 4.8 Hz, 2H), 4.02 (t, J = 4.8 Hz, 2H), 3.35 (s, 3H). | | |
| A47 | ¹H-NMR (400 MHz, CDCl₃) δ = 8.05 (s, 1H), 7.92(d, J = 7.6 Hz, 1H), 7.74 (d, J = 7.6 Hz, 1H), 7.64- | 518.1 | 517.9 |
| | 7.62 (m, 1H), 7.45 (d, J = 2.0 Hz, 1H), 7.33 (d, J = 2.4 Hz, 1H), 7.14 (d, J = 8.8 Hz, 1H), 6.92 (d, | | |
| | J = 8.8 Hz, 1H), 5.14 (s, 2H), 4.43 (t, J = 6.4 Hz, 1H), 3.88 (t, J = 6.4 Hz, 1H), 3.08 (s, 3H), 1.66 (s, | | |
| | 6H) | | |
| A48 | ¹H-NMR (400 MHz, CDCl₃) δ = 8.13 (br s, 1H), 7.45 (d, J = 2.4 Hz, 1H), 7.32 (d, J = 2.4 Hz, 1H), | 552.1 | 552.0 |
| | 7.22 (br t, J = 4.4 Hz, 1H), 7.13 (d, J = 8.8 Hz, 2H), 6.90 (d, J = 8.8 Hz, 2H), 5.16 (s, 2H), 4.43 (t, | | |
| | J = 6.0 Hz, 2H), 3.88 (t, J = 6.0 Hz, 2H), 3.51 (s, 3H), 1.65 (s, 6H) | | |
| A49 | ¹H-NMR (400 MHz, CDCl₃) δ = 8.54 (br s, 2H), 7.89 (s, 1H), 7.45 (d, J = 2.0 Hz, 1H), 7.31 (d, | 534.1 | 533.9 |
| | J = 2.4 Hz, 1H), 7.13 (d, J = 8.8 Hz, 2H), 6.92 (d, J = 8.4 Hz, 2H), 5.12 (s, 2H), 4.43 (t, J = 6.4 Hz, | | |
| | 2H), 3.88 (t, J = 6.0 Hz, 2H), 3.09 (s, 3H), 1.65 (s, 6H) | | |
| A50 | ¹H-NMR (400 MHz, CDCl₃) δ = 8.03 (s, 1H), 7.90 (d, J = 7.6 Hz, 1H), 7.67 (d, J = 6.4 Hz, 1H), | 519.1 | 518.9 |
| | 7.59-7.56 (m, 1H), 7.45 (d, J = 2.0 Hz, 1H), 7.31 (d, J = 2.0 Hz, 1H), 7.15 (d, J = 8.8 Hz, 2H), 6.92 | | |
| | (d, J = 8.4 Hz, 2H), 5.13 (s, 2H), 4.78 (br s, 2H), 4.43 (t, J = 6.4 Hz, 2H), 3.88 (t, J = 6.0 Hz, 2H), | | |
| | 1.66 (s, 6H) | | |
| A51 | ¹H-NMR (400 MHz, CDCl₃) δ = 9.43 (s, 1H), 8.16 (dd, J = 2.0, 8.8 Hz, 1H), 8.08 (d, J = 1.6 Hz, | 555.1 | 555.0 |
| | 1H), 7.97 (d, J = 8.8 Hz, 1H), 7.65 (d, J = 8.4 Hz, 2H), 7.52 (d, J = 2.4 Hz, 1H), 7.39 (d, J = 2.4 Hz, | | |
| | 1H), 7.34 (d, J = 8.4 Hz, 2H), 4.45 (t, J = 6.0 Hz, 2H), 3.90 (t, J = 6.4 Hz, 2H), 3.61 (s, 3H), 1.74 (s, | | |
| | 6H) | | |
| A52 | ¹H-NMR (400 MHz, CDCl₃) δ = 8.13 (s, 1H), 7.93 (s, 1H), 7.78 (d, J = 2.8 Hz, 1H), 7.64 (d, J = 2.8 | 505.0 | 522.18 (M + H₂O) |
| | Hz, 1H), 7.41 (d, J = 8.8 Hz, 1H), 7.24 (d, J = 3.2 Hz, 1H), 7.19 (d, J = 2.4 Hz, 1H), 6.98 (d, J = 2.4 | | |
| | Hz, 1H), 6.66 (d, J = 2.4 Hz, 1H), 5.06 (s, 2H), 4.53 (t, J = 6.0 Hz, 2H), 3.95 (t, J = 6.0 Hz, 2H), 3.36 | | |
| | (s, 3H) | | |
| A54 | ¹H-NMR (400 MHz, DMSO-d₆) δ = 8.11 (dd, J = 2.0, 11.2 Hz, 2H), 7.97 (s, 1H), 7.69 (d, J = 2.8 Hz, | 428.1 | 428.1 |
| | 1H), 7.54 (d, J = 9.2 Hz, 1H), 7.31 (d, J = 2.0 Hz, 1H), 6.93 (dd, J = 2.0, 8.8 Hz, 1H), 6.66 (d, J = 3.2 | | |
| | Hz, 1H), 5.20 (s, 2H), 4.50 (t, J = 5.2 Hz, 2H), 4.02 (t, J = 4.8 Hz, 2H) | | |
| A55 | ¹H-NMR (400 MHz, CDCl₃) δ = 7.77 (d, J = 2.4, 1H), 7.64 (d, J = 2.4, 1H), 7.39 (d, J = 8.8 Hz, 1H), | 522.1 | 521.1 |
| | 7.24 (d, J = 9.2 Hz, 1H), 7.12 (d, J = 2.0 Hz, 1H), 6.93 (d, J = 2.0 Hz, 1H), 6.64 (d, J = 3.2 Hz, 1H), | | |
| | 4.53 (t, J = 6.0 Hz, 2H), 3.96-3.88 (m, 6H), 2.81 (s, 3H), 2.73 (t, J = 12.0 Hz, 2H), 2.03-1.98 (m, | | |
| | 3H), 1.16-1.50 (m, 2H) | | |
| A56 | ¹H-NMR (400 MHz, CDCl₃) δ = 7.77 (d, J = 2.8, 1H), 7.63 (d, J = 2.4 Hz, 1H), 7.38 (d, J = 9.2 Hz, | 522.1 | 521.1 |
| | 1H), 7.22 (d, J = 3.2 Hz, 1H), 7.12 (d, J = 2.4 Hz, 1H), 6.93 (d, J = 2.4 Hz, 1H), 6.64 (d, J = 3.2 Hz, | | |
| | 1H), 4.53 (t, J = 6.0 Hz, 1H), 3.98-3.10 (m, 4H), 3.69 (d, J = 11.6 Hz, 1H), 2.82-2.70 (m, 5H), 2.26- | | |
| | 2.24 (m, 1H), 1.95-1.90 (m, 2H), 1.87-1.75 (m, 1H), 1.41-1.38 (m, 1H) | | |
| A57 | ¹H-NMR (400 MHz, DMSO-d₆) δ = 8.12 (d, J = 2.8 Hz, 1H), 8.10-8.08 (m, 1H), 7.99 (s, 1H), 7.71 | 520.1 | 520.0 |
| | (d, J = 3.2 Hz, 1H), 7.68 (d, J = 3.2 Hz, 1H), 7.52 (d, J = 9.2 Hz, 1H), 7.16 (d, J = 2.4 Hz, 1H), 6.88 | | |
| | (dd, J = 9.2, 2.4 Hz, 1H), 6.64 (d, J = 3.2 Hz, 1H), 4.53-4.48 (m, 2H), 4.13 (s, 2H), 4.05-4.00 (m, | | |
| | 2H), 2.91 (s, 3H), 1.97 (s, 6H) | | |
| A58 | ¹H-NMR (400 MHz, CDCl₃) δ = 7.77 (d, J = 2.8 Hz, 1H), 7.64 (d, J = 2.4 Hz, 1H), 7.38 (d, J = 9.2 | 536.1 | 553.23 (M + H₂O) |
| | Hz, 1H), 7.22 (d, J = 3.2 Hz, 1H), 7.11 (d, J = 2.0 Hz, 1H), 6.92 (d, J = 6.8 Hz, 1H), 6.64 (d, J = 2.8 | | |
| | Hz, 1H), 4.53(t, J = 6.0 Hz, 2H), 4.14-4.12 (m, 1H), 3.95 (t, J = 6.0 Hz, 2H), 3.84 (d, J = 6.0 Hz, 2H), | | |
| | 3.35-3.31 (m, 1H), 3.01 (s, 3H), 2.18-2.15 (m, 2H), 2.04-2.01 (m, 2H), 1.82-1.80 (m, 1H), 1.37- | | |
| | 1.22 (m, 4H) | | |
| A59 | ¹H-NMR (400 MHz, CDCl₃) δ = 7.78 (d, J = 2.4 Hz, 1H), 7.64 (d, J = 2.4 Hz, 1H), 7.40 (d, J = 9.2 | 494.1 | 494.0 |
| | Hz, 1H), 7.26-7.22 (m, 1H), 7.15 (d, J = 2.4 Hz, 1H), 6.93 (dd, J = 2.4, 8.8 Hz, 1H), 6.66 (d, J = 3.2 | | |
| | Hz, 1H), 4.56-4.51 (m, 2H), 4.17 (d, J = 6.0 Hz, 2H), 4.12 (t, J = 8.4 Hz, 2H), 3.96 (q, J = 6.0 Hz, | | |
| | 4H), 3.17-3.06 (m, 1H), 2.94 (s, 3H). | | |
| A60 | ¹H-NMR (400 MHz, CDCl₃) δ = 8.64 (d, J = 5.2 Hz, 1H), 8.14 (s, 1H), 8.06 (d, J = 2.0 Hz, 1H), 7.90 | 533.0 | 532.8 |
| | (d, J = 0.8 Hz, 1H), 7.67 (d, J = 8.8 Hz, 1H), 7.32-7.30 (m, 2H), 7.20 (s, 1H), 5.19 (s, 2H), 4.52 (t, | | |
| | J = 6.0 Hz, 2H), 3.95 (t, J = 6.0 Hz, 2H), 3.45 (s, 3H) | | |
| A61 | ¹H-NMR (400 MHz, DMSO-d₆) δ = 8.64 (d, J = 5.2 Hz, 1H), 8.17 (dd, J = 1.6, 3.6 Hz, 2H), 7.98 (s, | 549.0 | 548.9 |
| | 1H), 7.78 (d, J = 8.8 Hz, 1H), 7.73 (s, 1H), 7.24 (br d, J = 4.8 Hz, 1H), 7.17 (br d, J = 8.4 Hz, 1H), | | |
| | 5.24 (s, 2H), 5.50 (t, J = 5.2 Hz, 2H), 4.00 (t, J = 5.2 Hz, 2H), 3.36 (s, 3H). | | |
| A64 | ¹H-NMR (400 MHz, DMSO-d₆) δ = 8.67-8.63 (m, 2H), 8.43 (s, 1H), 8.14-8.07 (m, 3H), 7.35 (dd, | 533.0 | 533.0 |
| | J = 2.0, 9.6 Hz, 1H), 7.31 (d, J = 5.2 Hz, 1H), 5.25 (s, 2H), 4.47 (t, J = 5.6 Hz, 2H), 4.01(t, J = 5.6 Hz, | | |
| | 2H), 3.37 (s, 3H) | | |
| A66 | ¹H-NMR (400 MHz, CDCl₃) δ = 8.75-8.58 (m, 1H), 7.30 (br s, 2H), 7.24 (s, 1H), 7.14 (br d, | 534.1 | 534.0 |
| | J = 8.8 Hz, 2H), 6.89 (br d, J = 8.8 Hz, 2H), 5.12 (s, 2H), 5.09-4.93 (m, 1H), 4.05-3.96 (m, 2H), | | |
| | 3.82-3.76 (m, 2H), 3.47 (s, 3H), 1.62 (s, 6H). | | |
| A67 | ¹H-NMR (400 MHz, CDCl₃) δ = 8.64 (d, J = 5.2 Hz, 1H), 7.96 (d, J = 2.4 Hz, 1H), 7.29 (s, 1H), 7.22 | 504.2 | 504.1 |
| | (d, J = 2.0 Hz, 1H), 7.18 (d, J = 8.4 Hz, 2H), 6.87 (d, J = 8.4 Hz, 2H), 5.10 (s, 2H), 4.93 (t, J = 5.2 Hz, | | |
| | 1H), 3.47 (s, 3H), 3.28 (t, J = 6.8 Hz, 2H), 1.96-1.62 (m, 1H), 1.62 (s, 6H), 0.99 (d, J = 6.4 Hz, 6H). | | |
| A70 | ¹H-NMR (400 MHz, CDCl₃) δ = 8.62 (d, J = 5.2 Hz, 1H), 7.80 (d, J = 2.8 Hz, 1H), 7.68 (d, J = 2.4 | 552.1 | 552.0 |
| | Hz, 1H), 7.43 (d, J = 9.2 Hz, 1H), 7.33 (d, J = 5.2 Hz, 1H), 7.25 (d, J = 3.2 Hz, 1H), 7.16 (d, J = 2.4 | | |
| | Hz, 1H), 7.03 (dd, J = 2.4, 11.2 Hz, 1H), 6.67 (d, J = 3.6 Hz, 1H), 5.18(s, 2H), 4.64 (q, J = 8.0 Hz, | | |
| | 2H), 3.49 (s, 3H) | | |
| A71 | ¹H-NMR (400 MHz, CDCl₃) δ = 8.61 (d, J = 5.2 Hz, 1H), 8.44 (s, 1H), 7.75 (d, J = 2.8 Hz, 1H), 7.61 | 526.1 | 526.0 |
| | (d, J = 2.4 Hz, 1H), 7.42 (d, J = 9.6 Hz, 1H), 7.34 (d, J = 5.2 Hz, 1H), 7.24 (d, J = 3.2 Hz, 1H), 7.16 (d, | | |
| | J = 2.4 Hz, 1H), 7.02 (d, J = 8.8 Hz, 1H), 6.64 (d, J = 3.6 Hz, 1H), 5.18 (s, 2H), 4.06 (d, J = 6.4 Hz, | | |
| | 2H), 3.49 (s, 3H), 2.29-2.21 (m, 1H), 1.15 (d, J = 6.8 Hz, 6H) | | |
| A72 | ¹H-NMR (400 MHz, CDCl₃) δ = 8.59 (d, J = 5.2 Hz, 1H), 7.81 (d, J = 2.4 Hz, 1H), 7.67 (d, J = 2.4 | 576.1 | 576.0 |
| | Hz, 1H), 7.43 (d, J = 9.2 Hz, 1H), 7.32 (d, J = 5.2 Hz, 1H), 7.25 (d, J = 3.2 Hz, 1H), 7.16 (d, J = 2.4 | | |
| | Hz, 1H), 7.06-6.97 (m, 1H), 6.67 (d, J = 3.2 Hz, 1H), 5.17 (s, 2H), 4.71 (t, J = 5.6 Hz, 2H), 3.61 (t, | | |
| | J = 5.6 Hz, 2H), 3.49 (s, 3H), 3.22 (s, 3H). | | |
| A73 | ¹H-NMR (400 MHz, CDCl₃) δ = 8.62 (d, J = 5.2 Hz, 1H), 8.50 (s, 1H), 7.78 (d, J = 2.8 Hz, 1H), 7.64 | 540.1 | 540.0 |
| | (d, J = 2.4 Hz, 1H), 7.42 (d, J = 9.6 Hz, 1H), 7.34 (d, J = 5.2 Hz, 1H), 7.25 (d, J = 3.2 Hz, 1H), 7.12 (d, | | |
| | J = 2.4 Hz, 1H), 7.03 (d, J = 2.4 Hz, 1H), 6.66 (d, J = 3.2 Hz, 1H), 5.18 (s, 2H), 4.96 (t, J = 7.6 Hz, | | |
| | 2H), 4.72 (t, J = 6.0 Hz, 2H), 4.51 (d, J = 6.4 Hz, 2H), 3.63-3.53 (m, 1H), 3.49 (s, 3H) | | |
| A74 | ¹H-NMR (400 MHz, CDCl₃) δ = 8.60 (d, J = 5.2 Hz, 1H), 7.53 (d, J = 8.8 Hz, 1H), 7.40 (s, 1H), 7.33 | 491.1 | 490.9 |
| | (d, J = 5.2 Hz, 1H), 7.30 (d, J = 3.6 Hz, 1H), 7.21 (s, 1H), 7.14 (d, J = 2.0 Hz, 1H), 7.02 (s, 1H), 6.64 | | |
| | (d, J = 3.2 Hz, 1H), 5.17 (s, 2H), 4.67(s, 2H), 3.49 (s, 3H), 3.47 (s, 3H) | | |
| A75 | ¹H-NMR (400 MHz, DMSO-d₆) δ = 11.42 (br s, 1H), 8.67 (d, J = 5.2 Hz, 1H), 8.21-8.18 (m, 2H), | 523.1 | 532.0 |
| | 7.59 (d, J = 8.8 Hz, 2H), 7.28-7.23 (m, 3H), 6.90 (d, J = 3.2 Hz, 1H), 5.27 (s, 2H), 4.66-4.55 (m, | | |
| | 2H), 4.06-3.96 (m, 2H), 3.38 (s, 3H). | | |
| A76 | ¹H-NMR (400 MHz, CDCl₃) δ = 8.10 (s, 1H), 7.43 (d, J = 2.0 Hz, 1H), 7.34 (d, J = 2.0 Hz, 1H), 7.10 | 592.1 | 591.8 |
| | (d, J = 8.4 Hz, 2H), 6.93 (d, J = 8.8 Hz, 2H), 5.33-5.30 (m, 1H), 5.06-5.03 (m, 1H), 4.89-4.86 (m, | | |
| | 2H), 4.42 (t, J = 6.4 Hz, 2H), 3.88 (t, J = 6.0 Hz, 2H), 3.47 (s, 3H), 2.01 (s, 3H), 1.64 (s, 6H) | | |
| A77 | ¹H-NMR (400 MHz, DMSO-d₆) δ = 8.65 (d, J = 5.4 Hz, 1H), 8.29 (q, J = 2.0 Hz, 8.8 Hz, 2H), 8.10 | 547.1 | 547.0 |
| | (d, J = 4.8 Hz, 1H), 7.38-7.36 (m, 2H), 7.29 (d, J = 4.8 Hz, 1H), 7.04 (d, J = 2.4 Hz, 1H), 5.27 (s, | | |
| | 2H), 4.51 (t, J = 5.2 Hz, 2H), 4.07 (s, 3H), 4.01 (t, J = 5.2 Hz, 2H), 3.33 (s, 3H). | | |
| A78 | ¹H-NMR (400 MHz, CDCl₃) δ = 7.80-7.75 (m, 2H), 7.68-7.62 (m, 2H), 7.48 (d, J = 8.8 Hz, 1H), | 530.1 | 530.0 |
| | 7.28 (d, J = 3.2 Hz, 1H), 7.24 (dd, J = 1.2, 8.8 Hz, 1H), 6.72 (d, J = 3.2 Hz, 1H), 6.50 (d, J = 2.0 Hz, | | |
| | 1H), 6.03-5.95 (m, 1H), 5.23-5.09 (m, 1H), 4.58-4.50 (m, 4H), 3.95 (t, J = 6.0 Hz, 2H), 2.94 (d, | | |
| | J = 2.4 Hz, 1H), 2.86 (s, 3H). | | |
| A81 | ¹H-NMR (400 MHz, DMSO-d₆) δ = 8.11 (dd, J = 2.8 Hz, 12.8 Hz, 2H), 7.71 (d, J = 7.6 Hz, 1H), | 545.1 | 545.0 |
| | 7.56 (d, J = 8.8 Hz, 1H), 7.48 (t, J = 3.6 Hz, 2H), 7.12 (d, J = 8.8 Hz, 1H), 6.69 (d, J = 3.2 Hz, 1H), | | |
| | 6.06 (d, J = 2.4 Hz, 1H), 5.98 (dd, J = 2.4 Hz, 7.2 Hz, 1H), 4.51 (t, J = 4.8 Hz, 2H), 4.07-4.01 (m, | | |
| | 4H), 3.14 (s, 3H), 2.99 (t, J = 7.6 Hz, 2H) | | |
| A84 | ¹H-NMR (400 MHz, DMSO-d₆) δ = 8.94-8.82(m, 1H), 8.11 (dd, J = 2.4, 13.2 Hz, 2H), 7.69 (d, | 518.1 | 518.1 |
| | J = 3.2 Hz, 1H), 7.53 (d, J = 7.2 Hz, 2H), 7.42 (s, 1H), 7.31 (s, 1H), 7.05 (d, J = 8.8 Hz, 1H), 6.66 (t, | | |
| | J = 6.8 Hz, 1H), 4.51 (t, J = 4.8, 2H), 4.33 (t, J = 7.2 Hz, 2H), 4.03 (t, J = 5.2 Hz, 2H), 3.17 (t, J = 7.2 | | |
| | Hz, 2H), 2.79 (s, 3H). | | |
| A85 | ¹H-NMR (400 MHz, CDCl₃) δ = 9.67 (br s, 1H), 8.61 (s, 1H), 7.51 (d, J = 2.4 Hz, 1H)), 7.39 (d, | 535.1 | 535.0 |
| | J = 2.0 Hz, 1H), 7.25 (d, J = 6.0 Hz, 1H), 6.88 (d, J = 8.8 Hz, 1H), 6.82 (d, J = 6.4 Hz, 2H), 5.99 (s, | | |
| | 1H), 5.21 (dd, J = 2.0, 12.4 Hz, 1H), 5.11-5.06 (m, 3H), 4.36 (t, J = 6.0 Hz, 2H), 3.81 (t, J = 6.0 Hz, | | |
| | 2H), 3.39 (s, 3H) | | |
| A89 | ¹H-NMR (400 MHz, CDCl₃) δ = 8.65 (t, J = 4.8 Hz, 1 H), 7.51 (d, J = 2.4 Hz, 1H), 7.38 (d, J = 1.6 | 551.1 | 551.0 |
| | Hz, 1H), 7.29 (d, J = 5.2 Hz, 1H), 7.12 (d, J = 8.8 Hz, 2H), 6.93 (d, J = 8.4 Hz, 2H), 5.13 (s, 2H), | | |
| | 4.45 (t, J = 6.0 Hz, 2H), 4.01 (s, 2H), 3.89 (t, J = 6.4 Hz, 2H), 3.47 (s, 3H), 1.66 (s, 3H) | | |
| A90 | ¹H-NMR (400 MHz, DMSO-d₆) δ = 11.40 (br s, 1H), 8.64 (d, J = 5.2 Hz, 1H), 7.50 (d, J = 2.4 Hz, | 570.2 | 570.4 |
| | 1H), 7.47 (d, J = 2.4 Hz, 1H), 7.23 (s, 1H), 7.21 (d, J = 8.8 Hz, 2H), 6.98 (d, J = 8.8 Hz, 2H), 5.15 (s, | | |
| | 2H), 3.37 (s, 3H), 3.33-3.31 (m, 2H), 3.31-3.10 (m, 4H), 1.80-1.70 (m, 2H), 1.63 (s, 6H), 1.57- | | |
| | 1.42 (m, 1H), 1.41-1.21 (m, 2H). | | |
| A91 | ¹H-NMR (400 MHz, CDCl₃) δ = 8.50 (s, 1H), 8.25 (s, 1H), 7.47 (d, J = 1.6 Hz, 1H), 7.32 (d, J = 2.0 | 549.1 | 549.2 |
| | Hz, 1H), 7.14 (d, J = 8.8 Hz, 2H), 6.91 (d, J = 8.8, 2H), 4.98 (s, 2H), 4.43 (t, J = 6.4 Hz, 2H), 3.89 (t, | | |
| | J = 6.0 Hz, 2H), 3.49 (s, 3H), 2.57 (s, 3H), 1.66 (s, 6H) | | |
| A93 | ¹H-NMR (400 MHz, CDCl₃) δ = 8.61 (s, 1H), 7.46 (d, J = 2.4, 1H), 7.32 (d, J = 2.4 Hz, 1H), 7.14 | 537.1 | 537.0 |
| | (dd, J = 2.0, 6.8 Hz, 2H), 6.90 (t, J = 6.8 Hz, 2H), 6.35 (s, 1H), 4.99 (s, 2H), 4.43 (t, J = 6.4 Hz, 2H), | | |
| | 3.88 (t, J = 6.4 Hz, 2H), 3.49 (s, 5H), 3.03 (s, 3H), 1.66 (s, 6H). | | |
| A94 | ¹H-NMR (400 MHz, CDCl₃) δ = 8.58 (s, 1H), 8.24 (s, 1H), 7.45 (d, J = 2.4 Hz, 1H), 7.31 (d, J = 2.0 | 457.1 | 457.0 |
| | Hz, 1H), 7.14 (dd, J = 2.0 Hz, 6.8 Hz, 2H), 6.93 (d, J = 8.8 Hz, 2H), 5.38 (s, 2H), 4.99 (s, 2H), 4.43 | | |
| | (t, J = 6.4 Hz, 2H), 3.88 (t, J = 6.4 Hz, 2H), 3.03 (s, 3H), 1.65 (s, 6H). | | |
| A95 | ¹H-NMR (400 MHz, CDCl₃) δ = 8.00 (d, J = 2.4 Hz, 1H), 7.91 (d, J = 2.4 Hz, 1H), 7.44 (d, J = 2.4 | 457.1 | 457.0 |
| | Hz, 1H), 7.32 (d, J = 2.4 Hz, 1H), 7.10 (d, J = 8.8 Hz, 2H), 6.96 (d, J = 8.8 Hz, 2H), 5.21 (s, 2H), | | |
| | 5.10 (br s, 2H), 4.42 (t, J = 6.0 Hz, 2H), 3.88 (t, J = 6.0 Hz, 2H), 1.63 (s, 6H). | | |
| A97 | ¹H-NMR (400 MHz, CDCl₃) δ = 7.45 (d, J = 2.0, 1H), 7.31 (d, J = 2.4, 1H), 7.13 (d, J = 8.8 Hz, 2H), | 549.1 | 549.0 |
| | 6.94 (d, J = 6.4 Hz, 1H), 6.89 (d, J = 8.8 Hz, 2H), 6.59 (d, J = 6.8 Hz, 1H), 4.98 (s, 2H), 4.83 (s, 2H), | | |
| | 4.43 (t, J = 6.0 Hz, 2H), 3.88 (t, J = 6.0 Hz, 2H), 3.09 (s, 3H), 1.65 (d, J = 4.0, 6H). | | |
| A98 | ¹H-NMR (400 MHz, DMSO-d₆) δ = 8.38 (s, 1H), 8.17 (s, 1H), 7.64 (t, J = 4.4 Hz, 1H), 7.57 (d, | 457.1 | 457.3 |
| | J = 2.4 Hz, 1H), 7.18 (d, J = 3.6 Hz, 2H), 6.94 (d, J = 8.8 Hz, 2H), 5.58 (s, 2H), 5.06 (s, 2H), 4.41 (t, | | |
| | J = 5.2 Hz, 2H), 3.95 (t, J = 5.2 Hz, 2H), 3.09 (s, 3H), 1.63 (s, 6H). | | |
| A99 | ¹H-NMR (400 MHz, DMSO-d₆) δ = 7.96 (s, 1H), 7.62 (d, J = 2.4 Hz, 1H), 7.56 (d, J = 2.4 Hz, 1H), | 618.2 | 618.0 |
| | 7.19 (d, J = 8, 8 Hz, 2H), 6.95 (J = 8, 8 Hz, 2H), 4.88 (s, 2H), 4.41 (t, J = 4.8 Hz, 2H), 3.95 (t, J = 4.8 | | |
| | Hz, 2H), 3.69 (s, 4H), 3.07 (s, 3H), 1.63 (s, 6H), 1.61-1.51 (m, 4H), 1.23 (s, 2H). | | |
| A100 | 1H-NMR (400 MHz, DMSO-d₆) δ = 11.50 (br s, 1H), 8.07(s, 1H), 7.65 (s, 1H), 7.62 (s, 1H), 7.22 | 620.1 | 620.0 |
| | (d, J = 8.8 Hz, 2H), 6.95 (d, J = 8.8 Hz, 2H), 4.96 (s, 1H), 4.43 (t, J = 5.2 Hz, 2H), 3.76 (t, J = 5.2 Hz, | | |
| | 2H), 3.70 (s, 4H), 3.69 (s, 4H), 3.13 (s, 3H), 1.66 (s, 6H). | | |
| A102 | ¹H-NMR (400 MHz, CDCl₃) δ = 8.69 (d, J = 5.2 Hz, 1H), 7.43 (t, J = 2.4 Hz, 2H), 7.32 (d, J = 2.4 Hz, | 569.1 | 569.2 |
| | 1H), 7.24 (d, J = 2.4 Hz, 1H), 7.02 (d, J = 2.4 Hz, 1H), 6.89 (d, J = 8.8 Hz, 1H), 5.17 (s, 2H), 4.42 (t, | | |
| | J = 6.4 Hz, 2H), 3.89 (t, J = 6.4 Hz, 2H), 3.48 (s, 3H), 1.65 (s, 6H) | | |
| A103 | ¹H-NMR (400 MHz, CDCl₃) δ = 9.82 (br s, 1H), 8.67 (d, J = 4.8 Hz, 1H), 7.33-7.27 (m, 2H), 7.19- | 511.1 | 511.0 |
| | 7.15 (m, 3H), 6.91 (d, J = 8.8 Hz, 2H), 5.12 (s, 2H), 4.27 (t, J = 5.6 Hz, 2H), 3.93 (t, J = 5.6 Hz, 2H), | | |
| | 3.47 (s, 3H), 1.59 (s, 6H). | | |
| A104 | ¹H-NMR (400 MHz, DMSO-d₆) δ = 11.39 (s, 1H), 8.61 (d, J = 5.2 Hz, 1H), 7.98 (d, J = 2.4 Hz, 1H), | 491.1 | 491.0 |
| | 7.59 (d, J = 2.0 Hz, 1H), 7.19 (dd, J = 2.0, 4.8 Hz, 3H), 6.95 (d, J = 8.8 Hz, 2H), 5.12 (s, 2H), 4.25 (t, | | |
| | J = 6.8 Hz, 2H), 3.34-3.33 (m, 3H), 1.77-1.69 (m, 2H), 1.62 (s, 6H), 0.96 (t, J = 7.2 Hz, 3H). | | |
| A105 | ¹H-NMR (400 MHz, CDCl₃) δ = 8.61 (d, J = 5.2 Hz, 1H), 8.54 (s, 1H), 8.07 (d, J = 2.4 Hz, 1H), 7.36 | 516.2 | 516.1 |
| | (d, J = 2.4 Hz, 1H), 7.27 (s, 1H), 7.16 (d, J = 8.8 Hz, 2H), 6.87 (d, J = 8.8 Hz, 2H), 5.09 (s, 2H), 3.47 | | |
| | (s, 3H), 3.26-3.20 (m, 4H), 1.75-1.69 (m, 5H), 1.65-1.60 (m, 7H). | | |
| A106 | ¹H-NMR (400 MHz, CDCl₃) δ = 8.66 (d, J = 5.2 Hz, 1H), 7.96 (d, J = 2.0 Hz, 1H), 7.29 (s, 1H), 7.21 | 528.2 | 528.1 |
| | (d, J = 2.0 Hz, 1H), 7.15 (d, J = 8.8 Hz, 2H), 6.86 (d, J = 8.8 Hz, 2H), 5.10 (s, 2H), 4.16 (s, 4H), 3.46 | | |
| | (s, 3H), 2.20 (t, J = 7.6 Hz, 4H), 1.88-1.84 (m, 2H), 1.62 (s, 6H). | | |
| A107 | ¹H-NMR (400 MHz, CDCl₃) δ = 8.43 (br s, 1H), 8.16 (s, 1H), 7.90 (br d, J = 8.0 Hz, 1H), 7.63 (d, | 555.1 | 554.9 |
| | J = 8.4 Hz, 2H), 7.51 (d, J = 2.4 Hz, 1H), 7.39 (d, J = 2.4 Hz, 1H), 7.32 (d, J = 8.4 Hz, 2H), 4.45 (t, | | |
| | J = 6.0 Hz, 2H), 3.89 (t, J = 6.0 Hz, 2H), 3.26 (br s, 3H), 1.73 (s, 6H) | | |
| A108 | ¹H-NMR (400 MHz, CDCl₃) δ = 8.66 (s, 1H), 8.27-8.25 (m, 1H), 8.17 (s, 1H), 8.01 (d, J = 8.8 Hz, | 556.1 | 556.2 |
| | 1H), 7.73 (d, J = 8.4 Hz, 2H), 7.54-7.51 (m, 1H), 7.39-7.37 (m, 2H), 7.37 (s, 1H), 4.46 (t, J = 6.0 | | |
| | Hz, 2H), 3.90 (t, J = 6.0 Hz, 2H), 3.63 (s, 3H), 1.75 (s, 6H). | | |
| A110 | ¹H-NMR (400 MHz, CDCl₃) δ = 9.47 (s, 1H), 9.35 (s, 1H), 8.22 (d, J = 2.0 Hζ, IH), 8.20 (d, J = 2.0 | 462.1 | 462.2 |
| | Hz, 1H), 8.15 (d, J = 8.8 Hz, 1H), 7.60 (d, J = 8.4 Hz, 2H), 7.52 (d, J = 2.4 Hz, 1H), 7.39 (d, J = 2.4 | | |
| | Hz, 1H), 7.36 (s, 1H), 7.34 (s, 1H), 4.45 (t, J = 6.0 Hz, 2H), 3.89 (t, J = 6.0 Hz, 2H), 1.74 (s, 6H). | | |
| A111 | ¹H-NMR (400 MHz, CDCl₃) δ = 8.35 (s, 1H), 8.14-7.95 (m, 1 H), 7.95 (d, J = 1.2 Hz, 1 H), 7.81 | 528.1 | 528.1 |
| | (d, J = 8.8 Hz, 1 H), 7.58 (d, J = 8.4 Hz, 2 H), 7.52 (d, J = 2.4 Hz, 1 H), 7.39 (d, J = 2.4 Hz, 1 H), 7.30 | | |
| | (d, J = 8.8 Hz, 2 H), 4.45 (t, J = 6.0 Hz, 2 H), 3.89 (t, J = 6.0 Hz, 2 H), 3.30 (s, 3 H), 1.73 (s, 6 H) | | |
| A112 | ¹H-NMR (400 MHz, CDCl₃) δ = 8.54 (d, J = 7.2 Hz, 1H), 7.61-7.50 (m, 3H), 7.49 (s, 1H), 7.35 (s, | 543.1 | 543.0 |
| | 1H), 7.30 (d, J = 14.4 Hz, 2H), 7.17 (d, J = 8.8 Hz, 1H), 5.15 (s, 2H), 4.45 (t, J = 6.4 Hz, 2H), 3.89 (t, | | |
| | J = 6.4 Hz, 2H), 3.14 (s, 3H), 1.71 (s, 6H). | | |
| A113 | ¹H-NMR (400 MHz, DMSO-d₆) δ = 8.85 (d, J = 2.0 Hz, 1H), 8.00 (d, J = 1.6 Hz, 1H), 7.82 (d, J = 8.4 | 544.1 | 544.1 |
| | Hz, 2H), 7.72 (d, J = 2.0 Hz, 1H), 7.66 (d, J = 2.0 Hz, 1H), 7.48 (d, J = 9.2 Hz, 1H), 7.40 (d, J = 8.8 | | |
| | Hz, 2H), 4.43 (t, J = 4.8 Hz, 2H), 3.96 (t, J = 5.2 Hz, 2H), 3.37 (s, 3H), 1.70 (s, 6H). | | |
| A114 | ¹H-NMR (400 MHz, CDCl₃) δ = 7.56-7.52 (m, 3H), 7.47 (s, 1H), 7.40 (d, J = 2.0 Hz, 2H), 7.29 (d, | 543.1 | 543.0 |
| | J = 2.8 Hz, 2H), 7.22 (d, J = 2.8 Hz, 2H), 4.55 (s, 2H), 4.47 (t, J = 6.0 Hz, 2H), 3.92 (t, J = 6.0 Hz, | | |
| | 2H), 3.64 (t, J = 6.0 Hz, 2H), 3.09 (t, J = 6.0 Hz, 2H), 2.90 (s, 3H), 1.74 (s, 6H). | | |
| A115 | ¹H-NMR (400 MHz, CDCl₃) δ = 8.62 (d, J = 5.2 Hz, 1H), 7.68 (d, J = 9.2 Hz, 1H), 7.48 (d, J = 7.2 | 488.1 | 488.0 |
| | Hz, 1H), 7.34-7.32 (m, 2H), 7.13 (d, J = 2.4 Hz, 1H), 7.03 (s, 1H), 6.69 (d, J = 2.4 Hz, 1H), 6.65 (d, | | |
| | J = 3.2 Hz, 1H), 6.48 (d, J = 6.0 Hz, 1H), 5.18 (s, 2H), 4.18 (t, J = 6.4 Hz, 2H), 3.63 (t, J = 6.0 Hz, | | |
| | 2H), 3.49 (s, 3H), 2.32 (q, J = 6.0, 6.4 Hz, 2H). | | |
| A116 | ¹H-NMR (400 MHz, DMSO-d₆) δ = 9.51 (s, 1H), 8.37 (d, J = 2.0 Hz, 1H), 8.31-8.24 (m, 1H), 7.88 | 529.0 | 528.9 |
| | (d, J = 8.8 Hz, 3H), 7.66 (d, J = 2.8 Hz, 1H), 7.62 (d, J = 3.2 Hz, 1H), 7.24 (d, J = 8.8 Hz, 2H), 4.46- | | |
| | 4.39 (m, 2H), 4.01-3.95 (m, 2H), 3.44 (s, 3H) | | |
| A117 | ¹H-NMR (400 MHz, CDCl₃) δ = 9.70 (s, 1H), 7.75-7.73 (m, 1H), 7.52-7.50 (m, 2H), 7.37 (d, | 571.1 | 571.0 |
| | J = 2.4 Hz, 1H), 7.26 (d, J = 6.0 Hz, 2H), 7.08 (d, J = 8.8 Hz, 2H), 6.81 (d, J = 7.6 Hz, 1H), 4.45 (t, | | |
| | J = 6.4 Hz, 2H), 3.89 (t, J = 6.0 Hz, 2H), 3.57 (s, 3H), 1.71 (s, 6H). | | |
| A120 | ¹H-NMR (400 MHz, DMSO-d₆) δ = 7.99 (s, 1H), 7.64 (d, J = 2.4 Hz, 1 H), 7.57(d, J = 2.4 Hz, 1 H), | 619.2 | 619.4 |
| | 7.19 (d, J = 8.8 Hz, 2 H), 6.95 (d, J = 8.8 Hz, 2 H), 4.89 (s, 1H), 4.41 (t, J = 4.8 Hz, 2H), 3.95 (t, | | |
| | J = 4.8 Hz, 2H), 3.75-3.55 (m, 5H), 3.07 (s, 3H), 2.78 (t, J = 4.4 Hz, 4H), 1.63 (s, 6H). | | |
| A121 | ¹H-NMR (400 MHz, CDCl₃) δ = 9.30 (s, 1H), 8.06 (s, 1H), 7.95 (d, J = 8.4 Hz, 1H), 7.80-7.78 (m, | 555.1 | 554.9 |
| | 1H), 7.70 (d, J = 8.4 Hz, 2H), 7.51 (d, J = 2.0 Hz, 1H), 7.38 (d, J = 2.0 Hz, 1H), 7.35 (d, J = 8.4 Hz, | | |
| | 2H), 4.45 (t, J = 6.0 Hz, 2H), 3.90 (t, J = 6.0 Hz, 2H), 3.59 (s, 3H), 1.74 (s, 6H). | | |
| A122 | ¹H-NMR (400 MHz, DMSO-d₆) δ = 9.43 (s, 1H), 8.30 (s, 1 H), 8.27 (d, J = 3.6 Hz, 1H), 8.22-8.20 | 552.1 | 552.0 |
| | (m, 1H), 8.18 (d, J = 2.8 Hz, 1H), 8.09 (d, J = 0.8 Hz, 1H), 7.79 (d, J = 3.2 Hz, 1H), 7.78-7.74 (m, | | |
| | 1H), 7.74 (s, 1H), 7.70-7.68 (m, 1H), 6.84 (d, J = 3.2 Hz, 1H), 4.53 (t, J = 4.8 Hz, 2H), 4.03 (t, J = 5.2 | | |
| | Hz, 2H), 3.32-3.31 (m, 3H). | | |
| A123 | ¹H-NMR (400 MHz, CDCl₃) δ = 7.79 (d, J = 2.4 Hz, 1H), 7.66 (d, J = 2.8 Hz, 1H), 7.61 (s, 1H), 7.45 | 507.1 | 507.1 |
| | (d, J = 8.4 Hz, 1H), 7.26 (d, J = 3.2 Hz, 2H), 6.71 (d, J = 3.2 Hz, 1H), 4.54 (t, J = 6.0 Hz, 2H), 3.95 (t, | | |
| | J = 6.0 Hz, 2H), 3.67 (s, 2H), 3.27-3.25 (m, 4H), 2.79 (s, 3H), 2.61-2.59(m, 4H). | | |
| A124 | ¹H-NMR (400 MHz, CDCl₃) δ = 7.78 (d, J = 2.4 Hz, 1H), 7.64 (d, J = 2.4 Hz, 1H), 7.51 (s, 1H), 7.42 | 521.1 | 521.0 |
| | (d, J = 8.4 Hz, 1H), 7.24 (d, J = 3.6 Hz, 1H), 7.14 (d, J = 7.2 Hz, 1H), 6.68 (d, J = 3.2 Hz, 1H), 4.53 (t, | | |
| | J = 6.0 Hz, 2H), 3.95 (t, J = 6.0 Hz, 2H), 3.30 (t, J = 4.4 Hz, 4H), 2.95-2.91 (m, 2H), 2.80(s, 3H), | | |
| | 2.74-2.73 (m, 2H), 2.73-2.70 (m, 4H). | | |
| A125 | ¹H-NMR (400 MHz, CDCl₃) δ = 7.80 (d, J = 2.8 Hz, 1H), 7.66 (d, J = 2.4 Hz, 1H), 7.52 (d, J = 0.4 | 535.1 | 535.0 |
| | Hz, 1H), 7.43 (d, J = 8.4 Hz, 1H), 7.24 (d, J = 2.4 Hz, 1H), 7.14 (d, J = 8.4 Hz, 1H), 6.68 (d, J = 3.2 | | |
| | Hz, 1H), 4.54 (t, J = 6.0 Hz, 2H), 3.96-3.92 (m, 4H), 3.70 (t, J = 6.8 Hz, 2H), 3.38 (t, J = 4.8 Hz, 2H), | | |
| | 3.27 (t, J = 2.4 Hz, 2H), 3.02 (t, J = 7.6 Hz, 2H), 2.83 (s, 3H) | | |
| A126 | ¹H-NMR (400 MHz, DMSO-d₆) δ = 8.13 (d, J = 2.8 Hz, 1H), 8.10 (d, J = 2.8 Hz, 1H), 7.68 (d, | 507.1 | 507.0 |
| | J = 3.6 Hz, 1H), 7.56 (d, J = 8.4 Hz, 2H), 7.52 (d, J = 8.4 Hz, 1H), 7.08 (d, J = 7.2 Hz, 1H), 6.66 (d, | | |
| | J = 3.2 Hz, 1H), 4.50 (t, J = 5.2 Hz, 2H), 4.01 (t, J = 5.2 Hz, 2H), 3.89-3.82 (m, 1H), 3.61-3.57 (m, | | |
| | 2H), 2.85 (s, 1H), 2.83-2.79 (m, 2H), 2.66-2.60 (m, 4H) | | |
| A127 | ¹H-NMR (400 MHz, CDCl₃) δ = 7.45 (d, J = 2.0 Hz, 1H), 7.3l(d, J = 2.0 Hz, 1H), 7.13 (d, J = 8.8 Hz, | 555.1 | 554.8 |
| | 2H), 6.91 (d, J = 8.8 Hz, 2H), 6.82(s, 1H), 5.03 (s, 2H), 4.43 (t, J = 6.0 Hz, 2H), 3.89-3.87 (m, 5H), | | |
| | 1.65(s, 3H). | | |
| A128 | ¹H-NMR (400 MHz, CDCl₃) δ = 8.63 (d, J = 5.2 Hz, 1H), 7.82 (d, J = 2.8 Hz, 1H), 7.69 (d, J = 2.4 | 564.0 | 563.8 |
| | Hz, 2H), 7.42 (d, J = 5.2 Hz, 2H), 7.33 (d, J = 5.2 Hz, 2H), 7.26 (d, J = 3.2 Hz, 2H), 7.16 (d, J = 2.4 | | |
| | Hz, 2H), 7.02 (dd, J = 9.6, 10.4 Hz, 1H), 6.92 (s, 1H), 6.68 (d, J = 3.2 Hz, 2H), 5.19 (s, 2H), 3.49 (s, | | |
| | 3H). | | |
| A132 | ¹H-NMR (400 MHz, CDCl₃) δ = 7.44 (d, J = 2.4 Hz, 1H), 7.31 (d, J = 2.4 Hz, 1H), 7.08 (d, J = 9.2 | 523.1 | 540.2 (M + 18) |
| | Hz, 2H), 6.82 (d, J = 8.8 Hz, 2H), 4.88 (s, 1H), 4.42 (t, J = 6.0 Hz, 2H), 4.08 (s, 2H), 3.88 (t, J = 6.0 | | |
| | Hz, 2H), 3.01 (s, 3H), 2.13 (s, 6H), 1.64 (s, 6H). | | |
| A133 | ¹H-NMR (400 MHz, DMSO-d₆) δ = 9.52 (s, 1H), 8.14-8.11 (m, 2H), 8.03 (d, J = 1.2 Hz, 1H), 7.70 | 533.1 | 533.0 |
| | (d, J = 4.2 Hz, 1H), 7.57 (d, J = 9.2 Hz, 1H), 7.41 (d, J = 1.6 Hz, 1H), 6.71 (s, 1H), 4.53 (t, J = 6.8 Hz, | | |
| | 2H), 4.05 (t, J = 6.8 Hz, 2H), 2.95 (s, 3H), 2.27 (s, 6H). | | |
| A134 | ¹H-NMR (400 MHz, CDCl₃) δ = 8.12 (s, 1H), 7.80 (d, J = 2.4 Hz, 1H), 7.71 (d, J = 9.2 Hz, 1H), | 533.1 | 532.9 |
| | 7.66 (d, J = 2.4 Hz, 1H), 7.48 (d, J = 8.8 Hz, 1H), 7.32 (d, J = 3.2 Hz, 1H), 6.81 (d, J = 3.2 Hz, 1H), | | |
| | 6.61 (s, 1H), 4.97 (s, 1H), 4.56 (t, J = 6.0 Hz, 2H), 3.95 (t, J = 6.0 Hz, 2H), 3.04 (s, 3H), 2.54 (s, 6H) | | |
| A135 | ¹H-NMR (400 MHz, CDCl₃) δ = 7.78 (d, J = 2.4 Hz, 1H), 7.64 (d, J = 2.8 Hz, 1H), 7.39 (d, J = 8.8 | 508.1 | 508.0 |
| | Hz, 1H), 7.23 (d, J = 3.2 Hz, 1H), 7.14 (d, J = 2.4 Hz, 1H), 6.94 (dd, J = 8.8, 2.4 Hz, 1H), 6.65 (d, | | |
| | J = 3.2 Hz, 1H), 4.53 (t, J = 6.0 Hz, 2H), 4.46 (br d, J = 8.8 Hz, 1H), 4.24-4.17 (m, 1H), 4.07 (d, | | |
| | J = 6.4 Hz, 2H), 3.94 (d, J = 6.4 Hz, 2H), 2.96 (s, 3 H), 2.77-2.69 (m, 1H), 2.53-2.45 (m, 2H), 2.30- | | |
| | 2.19 (m, 2H). | | |
| A136 | ¹H-NMR (400 MHz, CDCl₃) δ = 7.78 (d, J = 2.8 Hz, 1H), 7.64 (d, J = 2.8 Hz, 1H), 7.40 (d, J = 8.8 | 508.1 | 508.0 |
| | Hz, 1H), 7.23 (d, J = 3.2 Hz, 1H), 7.12 (d, J = 2.4 Hz, 1H), 6.94 (dd, J = 2.4, 8.8 Hz, 1H), 6.65 (d, | | |
| | J = 3.2 Hz, 1H), 4.59 (br d, J = 9.2 Hz, 1H), 4.53 (t, J = 6.0 Hz, 2H), 4.00-3.87 (m, 5H), 2.97 (s, 3H), | | |
| | 2.73-2.59 (m, 2H), 2.57-2.45 (m, 1H), 2.08-1.97 (m, 2H). | | |
| A137 | ¹H-NMR (400 MHz, CDCl₃) δ = 7.78 (d, J = 2.8 Hz, 1H), 7.64 (d, J = 2.8 Hz, 1H), 7.41 (d, J = 9.2 | 512.1 | 511.9 |
| | Hz, 1H), 7.26 (d, J = 3.2 Hz, 1H), 7.18 (d, J = 2.4 Hz, 1H), 6.98 (dd, J = 2.4, 8.8 Hz, 1H), 6.67 (d, | | |
| | J = 3.6 Hz, 1H), 4.54 (t, J = 6.0 Hz, 2H), 4.35 (s, 1H), 4.31 (s, 1H), 4.25 (d, J = 3.6 Hz, 2H), 4.20 (s, | | |
| | 2H), 3.95 (t, J = 6.0 Hz, 2H), 2.97 (s, 3H). | | |
| A138 | ¹H-NMR (400 MHz, CDCl₃) δ = 7.88 (d, J = 2.4 Hz, 1H), 7.76 (d, J = 2.4 Hz, 1H), 7.68 (d, J = 8.8 | 537.0 | 536.1 |
| | Hz, 1H), 7.45 (s, 1H), 7.29-7.28 (m, 1H), 7.00 (dd, J = 2.4, 8.8 Hz, 1H), 4.90 (s, 1H), 4.50 (t, J = 6.0 | | |
| | Hz, 2H), 4.18 (s, 2H), 3.92 (t, J = 6.0 Hz, 2H), 3.02 (s, 3H), 2.16 (s, 6H). | | |
| A139 | ¹H-NMR (400 MHz, DMSO-d₆) δ = 8.04 (dd, J = 2.8, 13.2 Hz, 2H), 7.97 (s, 1H), 7.02 (dd, J = 2.4, | 534.1 | 534.1 |
| | 10.4 Hz, 2H), 6.72 (dd, J = 2.4, 8.8 Hz, 1H), 6.34 (s, 1H), 4.54 (t, J = 5.2 Hz, 2H), 4.08 (s, 2H), 4.02 | | |
| | (t, J = 5.2 Hz, 2H), 2.90 (s, 3H), 2.27 (s, 3H), 1.95 (s, 6H). | | |
| A141 | ¹H-NMR (400 MHz, CDCl₃) δ = 9.29 (s, 1H), 8.02 (dd, J = 1.6, 7.2 Hz, 1H), 7.97-7.95 (m, 2H), | 531.1 | 531.0 |
| | 7.89-7.55 (m, 2H), 7.53 (d, J = 2.0 Hz, 1H), 7.40 (d, J = 2.0 Hz, 1H), 5.57-5.53 (m, 1H), 4.45 (t, | | |
| | J = 6.0 Hz, 2H), 3.88 (t, J = 6.0 Hz, 2H), 3.58 (s, 3H), 1.98 (d, J = 6.8 Hz, 3H) | | |
| A142 | ¹H-NMR (400 MHz, CDCl₃) δ = 9.42 (s, 1H), 8.39 (dd, J = 2.0, 6.8 Hz, 1H), 8.29 (d, J = 2.0 Hz, | 531.1 | 531.0 |
| | 1H), 7.92 (d, J = 8.8 Hz, 1H), 7.55 (dd, J = 3.2, 7.2 Hz, 2H), 7.39 (d, J = 3.0 Hz, 1H), 6.76 (d, J = 3.0 | | |
| | Hz, 1H), 5.56-5.54 (m, 1H), 4.45 (t, J = 6.0 Hz, 2H), 3.88 (t, J = 6.0 Hz, 2H), 3.61 (s, 3H), 1.97 (d, | | |
| | J = 6.8 Hz, 3H). | | |
| A143 | ¹H-NMR (400 MHz, DMSO-d₆) δ = 8.65 (s, 1H), 8.32 (d, J = 2.4 Hz, 2H), 8.29 (d, J = 2.0 Hz, 1H), | 517.1 | 516.9 |
| | 8.15 (s, 1H), 7.85 (d, J = 8.4 Hz, 1H), 7.57 (d, J = 8.4 Hz, 1H), 5.69 (s, 2H), 4.50 (t, J = 5.2 Hz, 2H), | | |
| | 4.00 (t, J = 5.2 Hz, 2H), 3.30 (s, 3H). | | |
| A144 | ¹H-NMR (400 MHz, DMSO-d₆) δ = 8.61 (s, 2H), 8.30 (s, 1H), 8.15 (dd, J = 9.6, 2.0 Hz, 2H), 7.96 | 517.1 | 517.0 |
| | (d, J = 8.8 Hz, 1H), 7.55 (t, J = 7.2 Hz, 1H), 7.36 (d, J = 7.2 Hz, 1H), 5.71 (s, 2H), 4.53 (t, J = 5.2 Hz, | | |
| | 2H), 4.02 (t, J = 5.2 Hz, 2H), 3.30 (s, 3H). | | |
| A145 | ¹H-NMR (400 MHz, CDCl₃) δ = 8.02 (s, 1H), 7.79 (d, J = 2.4 Hz, 1H), 7.66 (d, J = 2.8 Hz, 1H), 7.63 | 519.1 | 519.0 |
| | (d, J = 0.8 Hz, 1H), 7.44 (d, J = 8.4 Hz, 1H), 7.26-7.23 (m, 2H), 6.69 (d, J = 3.2 Hz, 1H), 4.81 (br, | | |
| | J = 1.2 Hz, 1H), 4.54 (t, J = 6.0 Hz, 2H), 3.95 (t, J = 6.0 Hz, 2H), 3.91 (s, 2H), 2.98 (s, 3H), 2.12 (s, | | |
| | 6H). | | |
| A146 | ¹H-NMR (400 MHz, DMSO-d₆) δ = 8.06 (d, J = 2.8 Hz, 1H), 8.03 (d, J = 2.4 Hz, 1H), 7.91 (s, 1H), | 519.1 | 519.0 |
| | 7.54 (d, J = 3.2 Hz, 1H), 7.39 (br d, J = 9.6 Hz, 1H), 6.71-6.69 (m, 2H), 6.50 (d, J = 3.2 Hz, 1H), | | |
| | 5.26-5.19 (m, 1H), 4.48 (t, J = 4.8 Hz, 2H), 4.00 (t, J = 5.6 Hz, 2H), 3.20(s, 2H), 2.88(s, 3H), 1.90(s, | | |
| | 6H). | | |
| A148 | ¹H-NMR (400 MHz, DMSO-d₆) δ = 11.41(br s, 1H), 8.64(d, J = 5.2 Hz, 1H), 8.33-8.28 (m, 3H), | 543.1 | 543.0 |
| | 7.96 (d, J = 8.8 Hz, 1H), 7.91 (s, 2H), 7.49 (d, J = 2.4 Hζ, IH), 7.37 (d, J = 8.8 Hz, 1H), 7.26 (d, J = 5.2 | | |
| | Hz, 1H), 5.29 (s, 2H), 4.51 (t, J = 4.8 Hz, 2H), 4.01 (t, J = 5.2 Hz, 2H), 3.37 (s, 3H). | | |
| A150 | ¹H-NMR (400 MHz, MeOD) δ = 8.53 (d, J = 5.2 Hz, 1H), 7.21 (d, J = 5.2 Hz, 1H), 7.17-7.10 (m, | 506.1 | 506.0 |
| | 4H), 6.91-6.86 (m, 2H), 6.86-6.81 (m, 2H), 5.10 (s, 2H), 4.14-4.07 (m, 1H), 4.02 (dd, J = 1.6, 5.2 | | |
| | Hz, 2H), 3.79-3.73 (m, 1H), 3.70-3.63 (m, 1H), 3.36 (s, 3H), 1.61 (s, 6H) | | |
| A151 | ¹H-NMR (400 MHz, CDCl₃) δ = 7.45 (d, J = 2.0 Hz, 1H), 7.31 (d, J = 2.4 Hz, 1H), 7.10 (dd, J = 2.0 | 497.1 | 497.0 |
| | Hz, 6.8 Hz 2H), 6.84 (dd, J = 2.0 Hz, 6.8 Hz 2H), 4.42 (t, J = 6.4 Hz, 2H), 4.11-4.07 (m, 4H), 3.93- | | |
| | 3.86 (m, 4H), 3.09-3.05 (m, 1H), 2.92 (s, 3H), 1.65 (s, 6H) | | |
| A152 | ¹H-NMR (400 MHz, CDCl₃) δ = 9.29 (s, 1H), 7.97-7.93 (m, 2H), 7.83 (d, J = 2.0 Hz, 1H), 7.70 (d, | 543.1 | 543.0 |
| | J = 2.0 Hz, 1H), 7.62 (m, 1H), 7.42 (t, J = 8.0 Hz, 1H), 7.16-7.14 (m, 2H), 7.06-7.04 (m, 1H), 5.35 | | |
| | (s, 2H), 4.51 (t, J = 6.0 Hz, 2H), 3.93 (t, J = 6.0 Hz, 2H), 3.57 (s, 3H). | | |
| A153 | ¹H-NMR (400 MHz, CDCl₃) δ = 8.37 (s, 1H), 7.80 (d, J = 2.4 Hz, 1H), 7.68-7.66 (m, 2H), 7.47 (d, | 571.1 | 571.0 |
| | J = 8.8 Hz, 1H), 7.33 (br d, J = 8.8 Hz, 1H), 7.27 (s, 1H), 6.72 (d, J = 3.2 Hz, 1H), 4.54 (t, J = 6.0 Hz, | | |
| | 2H), 3.95 (t, J = 6.0 Hz, 2H), 3.68 (br s, 2H), 3.87 (br s, 2H), 3.40 (s, 3H), 2.84 (br s, 4H) | | |
| A154 | ¹H-NMR (400 MHz, CDCl₃) δ = 8.22 (s, 1H), 7.81 (d, J = 2.4 Hz, 1H), 7.67 (d, J = 2.8 Hz, 2H), 7.46 | 571.1 | 571.1 |
| | (d, J = 8.4 Hz, 1H), 7.31 (d, J = 8.0 Hz, 1H), 7.27-7.20 (m, 1H), 6.72 (d, J = 3.2 Hz, 1H), 4.54 (t, | | |
| | J = 6.0 Hz, 2H), 3.95 (t, J = 6.0 Hz, 2H), 3.84 (s, 2H), 3.57 (s, 2H), 3.43 (s, 3H), 3.02-2.94 (m, 2H), | | |
| | 2.92-2.84 (m, 2H). | | |
| A155 | ¹H-NMR (400 MHz, CDCl₃) δ = 8.38 (s, 1H), 7.47 (d, J = 2.0 Hz, 1H), 7.33 (d, J = 2.0 Hz, 1H), | 560.1 | 560.0 |
| | 7.27-7.11 (m, 2H), 6.94 (br d, J = 8.8 Hz, 2H), 4.44-4.41 (m, 2H), 4.34 (s, 2H), 3.88 (t, J = 6.0 Hz, | | |
| | 2H), 3.65-3.62 (m, 3H), 3.48 (s, 3H), 3.07-3.04 (m, 2H), 1.65 (s, 6H). | | |
| A157 | ¹H-NMR (400 MHz, CDCl₃) δ = 8.63 (d, J = 5.2 Hz, 1H), 7.85 (d, J = 2.0 Hz, 1H), 7.75-7.71 (m, | 546.1 | 546.0 |
| | 2H), 7.37 (d, J = 5.2 Hz, 1H), 7.19 (s, 1H), 6.98 (dd, J = 8.8, 2.4 Hz, 1H), 6.90 (d, J = 2.4 Hz, 1H), | | |
| | 5.22 (s, 2H), 4.48 (t, J = 6.0 Hz, 2H), 3.93 (t, J = 6.0 Hz, 2H), 3.81 (s, 3H), 3.49 (s, 3H). | | |
| A158 | ¹H-NMR (400 MHz, CDCl₃) δ = 7.78 (d, J = 2.4 Hz, 1H), 7.65 (d, J = 2.4 Hz, 1H), 7.41 (d, J = 8.4 | 510.1 | 510.0 |
| | Hz, 1H), 7.26 (s, 1H), 7.19 (d, J = 2.8 Hz, 1H), 6.96 (dd, J = 9.2, 2.4 Hz, 1H), 6.67 (d, J = 3.2 Hz, | | |
| | 1H), 4.54 (t, J = 6.0 Hz, 2H), 4.26 (s, 2H), 4.03 (s, 4H), 3.95 (t, J = 6.0 Hz, 2H), 3.07 (s, 1H), 2.95 | | |
| | (s, 3H). | | |
| A159 | ¹H-NMR (400 MHz, CDCl₃) δ = 7.78 (d, J = 2.8 Hz, 1H), 7.64 (d, J = 2.4 Hz, 1H), 7.40 (d, J = 8.8 | 508.1 | 508.0 |
| | Hz, 1H), 7.25 (d, J = 3.2 Hz, 1H), 7.18 (d, J = 2.4 Hz, 1H), 6.92 (dd, J = 9.2, 2.4 Hz, 1H), 6.65 (d, | | |
| | J = 3.2 Hz, 1H), 4.55-4.51 (m, 3H), 4.06-4.02 (m, 3H), 3.96-3.93 (m, 3H), 2.91 (s, 4H), 1.27 (d, | | |
| | J = 6.0 Hz, 3H). | | |
| A160 | ¹H-NMR (400 MHz, DMSO-d₆) δ = 8.70 (s, 2H), 8.58 (s, 1H), 8.21 (dd, J = 2.0 Hz, 4.8 Hz, 2H), | 517.1 | 516.9 |
| | 8.00 (s, 1H), 7.83(d, J = 8.8 Hz, 1H), 7.43(d, J = 8.8 Hz, 1H), 5.67 (s, 2H), 4.49 (t, J = 5.2 Hz, 2H), | | |
| | 4.00 (t, J = 5.2 Hz, 2H), 3.35 (s. 3H) | | |
| A161 | ¹H-NMR (400 MHz, DMSO-d₆) δ = 8.83 (s, 1H), 8.72 (s, 2H), 8.18 (dd, J = 9.2, 2.4 Hz, 2H), 7.67 | 517.1 | 517.0 |
| | (d, J = 8.8 Hz, 1H), 7.35 (t, J = 6.8 Hz, 1H), 7.27 (d, J = 8.8 Hz, 1H), 5.67 (s, 2H), 4.53 (t, J = 5.2 Hz, | | |
| | 2H), 4.03 (t, J = 5.2 Hz, 2H), 3.33 (s, 3H). | | |
| A162 | ¹H-NMR (400 MHz, CDCl₃) δ = 7.79 (d, J = 2.4 Hz, 1H), 7.65 (d, J = 2.4 Hz, 1H), 7.40 (d, J = 8.8 | 534.1 | 534.0 |
| | Hz, 1H), 7.24 (d, J = 3.2 Hz, 1H), 7.13 (d, J = 2.4 Hz, 1H), 6.93 (dd, J = 2.4, 8.8 Hz, 1H), 6.66 (d, | | |
| | J = 3.2 Hz, 1H), 4.54 (t, J = 6.0 Hz, 2H), 3.98-3.94 (m, 8H), 2.87 (s, 3H), 2.70 (td, J = 6.8, 13.6 Hz, | | |
| | 1H), 2.44 (dd, J = 8.4, 12.8 Hz, 2H), 2.24-2.19 (m, 2H). | | |
| A163 | ¹H-NMR (400 MHz, CDCl₃) δ = 7.77 (d, J = 2.8 Hz, 1H), 7.63 (d, J = 2.4 Hz, 1H), 7.38 (d, J = 9.2 | 520.1 | 520.3 |
| | Hz, 1H), 7.25-7.22 (m, 1H), 6.98 (d, J = 2.4 Hz, 1H), 6.92 (d, J = 4.0 Hz, 1H), 6.64 (d, J = 3.2 Hz, | | |
| | 1H), 4.69-4.64 (m, 1H), 4.53 (t, J = 6.0 Hz, 2H), 4.03-3.93 (m, 6 H), 2.87 (s, 3 H), 2.82-2.77 (m, | | |
| | 2H), 2.46-2.42 (m, 2H) | | |
| A164 | ¹H-NMR (400 MHz, CDCl₃) δ = 7.90 (s, 1H), 7.83 (d, J = 2.4 Hz, 1H), 7.68 (q, J = 2.4, 5.2 Hz, 3H), | 514.1 | 514.0 |
| | 7.55 (q, J = 8.4, 13.6 Hz, 2H), 7.45 (d, J = 8.0 Hz, 2H), 7.30 (d, J = 3.2 Hz, 1H), 6.80 (d, J = 3.2 Hz, | | |
| | 1H), 4.61 (s, 1H), 4.55 (t, J = 6.0 Hz, 2H), 4.40 (d, J = 6.0 Hz, 2H), 3.96 (t, J = 6.0 Hz, 2H), 2.95 (s, | | |
| | 3H) | | |
| A166 | ¹H-NMR (400 MHz, CDCl₃) δ = 8.73 (br s, 1H), 8.63 (d, J = 5.2 Hz, 1H), 8.14 (d, J = 2.4 Hz, 1H), | 519.2 | 519.1 |
| | 7.46 (d, J = 2.4 Hz, 1H), 7.28 (s, 1H), 7.14 (d, J = 9.2 Hz, 2H), 6.87 (d, J = 8.8 Hz, 2H), 5.09 (s, 2H), | | |
| | 4.27 (s, 4H), 3.47 (s, 3H), 2.23 (t, J = 7.6 Hz, 4H), 1.88 (q, J = 8.8, 15.2 Hz, 2H), 1.62 (s, 6H) | | |
| A167 | ¹H-NMR (400 MHz, CDCl₃) δ = 7.80 (d, J = 2.8 Hz, 1H), 7.67 (d, J = 2.8 Hz, 1H), 7.39 (d, J = 9.2 | 540.1 | 540.1 |
| | Hz, 1H), 7.23 (d, J = 3.6 Hz, 1H), 7.10 (d, J = 2.4 Hz, 1H), 6.93 (d, J = 8.8, 2.4 Hz, 1H), 6.65 (d, | | |
| | J = 3.2 Hz, 1H), 4.88 (s, 1H), 4.64 (q, J = 8.0 Hz, 2H), 4.16 (s, 2H), 3.01 (s, 3H), 2.16 (s, 6H). | | |
| A168 | ¹H-NMR (400 MHz, CDCl₃) δ = 7.89 (s, 1H), 7.83 (d, J = 2.4 Hz, 1H), 7.69 (d, J = 2.4 Hz, 1H), | 528.1 | 528.0 |
| | 7.58-7.48 (m, 4H), 7.41 (d, J = 8.0 Hz, 1H), 7.30 (d, J = 3.2 Hz, 1H), 6.79 (d, J = 3.2 Hz, 1H), 4.55 (t, | | |
| | J = 6.0 Hz, 2H), 4.41 (m, 3H), 3.96 (t, J = 6.0 Hz, 2H), 2.95 (s, 3H), 2.49 (s, 3H) | | |
| A169 | ¹H-NMR (400 MHz, MeOD-d₆) δ = 8.48 (s, 1H), 8.00 (d, J = 2.4 Hz, 1H), 7.90 (d, J = 2.4 Hz, 1H), | 521.1 | 521.0 |
| | 7.71 (s, 1H), 7.62-7.54 (m, 2H), 7.31 (dd, J = 8.4, 1.6 Hz, 1H), 6.76 (d, J = 3.2 Hz, 1H), 4.56 (t, | | |
| | J = 6.0 Hz, 2H), 4.04 (br s, 2H), 3.97 (t, J = 5.6 Hz, 2H), 3.44-3.41 (m, 1H), 3.20 (br d, J = 11.2 Hz, | | |
| | 2H), 2.96 (s, 3H), 2.83-2.51 (m, 2H), 2.07 (br d, J = 11.2 Hz, 2H), 1.72 (br d, J = 11.2 Hz, 2H). | | |
| A170 | ¹H-NMR (400 MHz, CDCl₃) δ = 7.80 (d, J = 2.8 Hz, 1H), 7.66 (d, J = 2.8 Hz, 1H), 7.63 (s, 1H), 7.45 | 537.1 | 537.1 |
| | (d, J = 8.4 Hz, 1H), 7.28 (d, J = 1.2 Hz, 1H), 7.26 (br s, 1H), 6.72 (d, J = 3.2 Hz, 1H), 4.75-4.60 (m, | | |
| | 1H), 4.54 (t, J = 6.0 Hz, 2H), 3.95 (t, J = 6.0 Hz, 2H), 3.94-3.93 (m, 1H), 3.93-3.90 (m, 2H), 3.89- | | |
| | 3.87(m, 1H), 3.29-3.21 (m, 1H), 3.16-3.09 (m, 1H), 2.96 (s, 3H), 2.91-2.78 (m, 2H), 2.41-2.24 | | |
| | (m, 1H), 2.21-2.03 (m, 1H). | | |
| A171 | ¹H-NMR (400 MHz, CDCl₃) δ = 8.46 (s, 1H), 7.79 (d, J = 2.4 Hz, 1H), 7.65 (d, J = 2.4 Hz, 1H), 7.63 | 533.1 | 533.0 |
| | (s, 1H), 7.46 (d, J = 8.8 Hz, 1H), 7.28 (s, 1H), 7.24 (d, J = 1.6 Hz, 1H), 6.73 (d, J = 3.6 Hz, 1H), 4.71- | | |
| | 4.60 (m, 1H), 4.55 (t, J = 6.0 Hz, 2H), 4.96-3.91 (m, 4H), 3.85-3.77 (m, 1H), 3.63 (s, 2H), 3.57 (s, | | |
| | 2H), 2.91 (s, 3H), 2.68 (ddd, J = 3.2, 7.2, 10.4 Hz, 2H), 2.13 (d, J = 3.2 Hz, 2H). | | |
| A173 | ¹H-NMR (400 MHz, DMSO-d₆) δ = 8.78 (br s, 1H), 8.66 (br s, 1H), 8.49 (s, 1H), 8.06 (br d, J = 8.8 | 570.1 | 570.0 |
| | Hz, 1H), 7.71-7.69 (m, 3H), 7.63 (d, J = 2.4 Hz, 1H), 7.38 (br d, J = 8.4 Hz, 3H), 4.43 (t, J = 5.2 Hz, | | |
| | 2H), 3.96 (t, J = 5.2 Hz, 2H), 3.12 (s, 3H), 1.71 (s, 6H). | | |
| A174 | ¹H-NMR (400 MHz, MeOD) δ = 8.54 (d, J = 4.8 Hz, 1H), 7.49-7.41 (m, 2H), 7.23 (d, J = 5.2 Hz, | 545.2 | 545.1 |
| | 1H), 7.18 (d, J = 9.2 Hz, 2H), 6.96 (d, J = 8.8 Hz, 2H), 5.13 (s, 2H), 4.10-4.08 (m, 1H), 3.93-3.84 | | |
| | (m, 1H), 3.76 (dd, J = 5.2, 1.1 Hz, 1H), 3.37 (s, 3H), 1.91-1.70 (m, 2H), 1.65 (s, 6H), 1.56 (br d, | | |
| | J = 8.0 Hz, 1H), 1.04 (q, J = 7.2 Hz, 3H) | | |
| A176 | ¹H-NMR (400 MHz, CDCl₃) δ = 9.10 (br s, 1H), 8.07-7.97 (m, 1H), 7.90 (s, 1H), 7.73 (br d, J = 8.8 | 505.1 | 505.0 |
| | Hz, 1H), 7.60 (d, J = 8.4 Hz, 2H), 7.50 (d, J = 2.0 Hz, 1H), 7.38 (d, J = 2.0 Hz, 1H), 7.30 (d, J = 8.4 | | |
| | Hz, 2H), 4.45 (t, J = 6.0 Hz, 2H), 3.89 (t, J = 6.0 Hz, 2H), 3.68-3.63 (m, 2H), 1.73 (s, 6H), 1.35 (t, | | |
| | J = 7.2 Hz, 3H). | | |
| A177 | ¹H-NMR (400 MHz, CDCl₃) δ = 9.17 (s, 1H), 8.06 (dd, J = 2.0, 8.4 Hz, 1H), 7.95 (d, J = 2.0 Hz, | 477.1 | 477.1 |
| | 1H), 7.75 (d, J = 8.4 Hz, 1H), 7.61 (d, J = 8.4 Hz, 2H), 7.51 (d, J = 2.4 Hz, 1H), 7.38 (d, J = 2.4 Hz, | | |
| | 1H), 7.31 (d, J = 8.4 Hz, 2H), 6.03-5.75 (s, 1H), 4.45 (t, J = 6.0 Hz, 2H), 3.89 (t, J = 6.0 Hz, 2H), | | |
| | 1.73 (s, 6H). | | |
| A180 | ¹H-NMR (400 MHz, CDCl₃) δ = 7.78 (d, J = 2.8 Hz, 1H), 7.64 (d, J = 2.0 Hz, 1H), 7.37 (d, J = 8.8 | 534.1 | 534.0 |
| | Hz, 1H), 7.22 (d, J = 3.2 Hz, 1H), 7.14 (d, J = 2.4 Hz, 1H), 6.91 (dd, J = 2.4, 8.8 Hz, 1H), 6.63 (d, | | |
| | J = 3.2 Hz, 1H), 4.85 (s, 1H), 4.56-4.51 (m, 3H), 3.95 (t, J = 6.0 Hz, 2H), 2.99 (s, 3H), 2.09-2.02 (m, | | |
| | 6H), 1.31 (d, J = 6.0 Hz, 3H). | | |
| A181 | ¹H-NMR (400 MHz, DMSO-d₆) δ = 11.42 (s, 1H), 8.61 (d, J = 4.8 Hz, 1H), 7.63 (d, J = 2.4 Hz, | 549.1 | 549.0 |
| | 1H), 7.56 (d, J = 2.4 Hz, 1H), 7.19-7.17 (m, 3H), 6.95 (d, J = 8.8 Hz, 2H), 5.12 (s, 2H), 4.25 (t, | | |
| | J = 6.0 Hz, 2H), 3.85 (t, J = 6.0 Hz, 2H), 3.49-3.47 (m, 3H), 2.20 (t, J = 6.4 Hz, 2H), 1.62 (s, 6H). | | |
| A184 | ¹H-NMR (400 MHz, CDCl₃) δ = 7.80 (d, J = 2.4 Hz, 1H), 7.66 (d, J = 2.0 Hz, 2H), 7.46 (d, J = 8.4 | 533.1 | 533.2 |
| | Hz, 1H), 7.31 (d, J = 8.8 Hz, 1H), 7.27-7.26 (m, 1H), 6.71 (d, J = 3.6 Hz, 1H), 4.94 (s, 1H), 4.54 (t, | | |
| | J = 6.0 Hz, 2H), 4.08 (q, J = 6.4 Hz, 1H), 3.95 (t, J = 6.0 Hz, 2H), 2.89 (s, 3H), 2.06 (d, J = 9.2 Hz, | | |
| | 3H), 1.92 (d, J = 9.2 Hz, 3H), 1.51 (d, J = 6.4 Hz, 3H). | | |
| A187 | ¹H-NMR (400 MHz, CDCl₃) δ = 7.78 (d, J = 2.4 Hz, 1H), 7.64 (d, J = 2.4 Hz, 1H), 7.52 (s, 1H), 7.42 | 535.1 | 535.0 |
| | (d, J = 8.4 Hz, 1H), 7.24 (d, J = 3.6 Hz, 1H), 7.16-7.11 (m, 1H), 6.68 (d, J = 3.6 Hz, 1H), 4.76 (br d, | | |
| | J = 7.2 Hz, 1H), 4.54 (t, J = 6.0 Hz, 2H), 3.95 (t, J = 6.0 Hz, 2H), 3.52-3.45 (m, 1H), 3.25 (br d, | | |
| | J = 12.4 Hz, 2H), 3.09-3.03 (m, 2H), 3.02-2.99 (m, 3H), 2.92-2.86 (m, 2H), 2.48 (br t, J = 10.4 Hz, | | |
| | 2H), 2.13 (br d, J = 10.8 Hz, 2H), 1.97-1.87 (m, 2H) | | |
| A189 | ¹H-NMR (400 MHz, DMSO-d₆) δ = 8.59 (d, J = 4.8 Hz, 1H), 8.08 (dd, J = 2.4, 15.6 Hz, 2H), 7.69 | 524.1 | 524.0 |
| | (d, J = 3.2 Hz, 1H), 7.56 (d, J = 9.2 Hz, 1H), 7.24 (d, J = 2.4 Hz, 1H), 7.18 (br d, J = 4.8 Hz, 1H), 6.99 | | |
| | (dd, J = 2.8, 9.2 Hz, 1H), 6.64 (d, J = 3.2 Hz, 1H), 5.16 (s, 2H), 4.86-4.79 (m, 1H), 3.31 (s, 3H), | | |
| | 2.38-2.36 (m, 2H), 2.35-2.34 (m, 2H), 1.79-1.76 (m, 1H), 1.55-1.50 (m, 1H). | | |
| A190 | NA | 536.1 | 536.0 |
| A191 | ¹H-NMR (400 MHz, CDCl₃) δ = 8.27 (s, 1H), 8.03 (d, J = 8.4 Hz, 2H), 7.99 (d, J = 8.8 Hz, 1H), | 555.1 | 555.1 |
| | 7.91-7.83 (m, 1H), 7.47 (d, J = 2.0 Hz, 1H), 7.38 (d, J = 2.4 Hz, 1H), 7.36 (d, J = 8.4 Hz, 2H), 7.19- | | |
| | 7.11 (m, 1H), 4.44 (t, J = 6.0 Hz, 2H), 3.89 (t, J = 6.0 Hz, 2H), 3.22 (s, 3H), 1.73 (s, 6H). | | |
| A192 | ¹H-NMR (400 MHz, CDCl₃) δ = 9.48 (s, 1H), 9.44 (br s, 1H), 8.40 (d, J = 2.4 Hz, 1H), 7.66 (d, | 556.1 | 556.0 |
| | J = 8.4 Hz, 2H), 7.51 (d, J = 2.4 Hz, 1H), 7.42-7.35 (m, 3H), 4.46 (t, J = 6.0 Hz, 2H), 3.90 (t, J = 6.0 | | |
| | Hz, 2H), 3.63 (s, 3H), 1.74 (s, 6H) | | |
| A194 | ¹H-NMR (400 MHz, CDCl₃) δ = 8.88 (s, 1H), 8.11 (s, 1H), 7.89 (d, J = 9.6 Hz, 1H), 7.57 (d, J = 8.4 | 555.1 | 555.0 |
| | Hz, 2H), 7.50 (d, J = 2.4 Hz, 1H), 7.39-7.31 (m, 3H), 7.06-6.91 (m, 1H), 4.45 (t, J = 6.0 Hz, 2H), | | |
| | 3.89 (t, J = 6.0 Hz, 2H), 3.16 (s, 3H), 1.73 (s, 6H) | | |
| A195 | ¹H-NMR (400 MHz, CDCl₃) δ = 8.87 (s, 1H), 8.39 (s, 2H), 7.61 (d, J = 8.4 Hz, 2H), 7.50 (d, J = 2.0 | 556.1 | 556.0 |
| | Hz, 1H), 7.38-7.35 (m, 4H), 4.45 (t, J = 6.4 Hz, 2H), 3.89 (t, J = 6.0 Hz, 2H), 3.22 (s, 3H), 1.73 (s, | | |
| | 6H). | | |
| A196 | ¹H-NMR (400 MHz, CDCl₃) δ = 7.92 (dd, J = 1.6, 8.8 Hz, 1H), 7.87 (d, J = 2.0 Hz, 1H), 7.76 (d, | 640.1 | 640.1 |
| | J = 8.4 Hz, 1H), 7.52 (d, J = 2.4 Hz, 1H), 7.49 (d, J = 8.4 Hz, 2H), 7.35 (d, J = 2.4 Hz, 1H), 7.30 (d, | | |
| | J = 8.4 Hz, 2H), 4.45 (t, J = 6.0 Hz, 2H), 4.12 (t, J = 4.4 Hz, 4H), 3.89 (m, J = 2.0, 6.0 Hz, 6H), 3.28 | | |
| | (s, 3H), 1.72 (s, 6H) | | |
| A197 | ¹H-NMR (400 MHz, CDCl₃) δ = 11.84 (s, 1H), 8.44 (d, J = 8.4 Hz, 1H), 8.07 (s, 1H), 7.98 (d, | 555.1 | 555.0 |
| | J = 1.6 Hz, 1H), 7.82 (dd, J = 2.0, 8.8 Hz, 1H), 7.68 (d, J = 8.4 Hz, 2H), 7.50 (d, J = 2.4 Hz, 1H), 7.38 | | |
| | (d, J = 2.4 Hz, 1H), 7.34 (d, J = 8.4 Hz, 2H), 4.45 (t, J = 6.0 Hz, 2H), 3.89 (t, J = 6.0 Hz, 2H), 3.21 (s, | | |
| | 3H), 1.73 (s, 6H) | | |
| A198 | ¹H-NMR (400 MHz, CDCl₃) δ = 11.55 (s, 1H), 10.36 (s, 1H), 9.20 (s, 1H), 7.58 (d, J = 8.0 Hz, 2H), | 544.1 | 544.0 |
| | 7.53 (d, J = 1.2 Hz, 1H), 7.40 (s, 2H), 7.29 (d, J = 8.0 Hz, 2H), 4.45 (t, J = 6.0 Hz, 2H), 3.90 (t, J = 6.0 | | |
| | Hz, 2H), 3.64 (s, 3H), 1.73 (s, 6H). | | |
| A199 | ¹H-NMR (400 MHz, CDCl₃) δ = 9.14 (d, J = 2.0 Hz, 1H), 8.21 (d, J = 2.0 Hz, 1H), 8.13-8.11 (m, | 555.1 | 555.0 |
| | 1H), 7.67 (d, J = 8.4 Hz, 2H), 7.51 (d, J = 2.0 Hz, 1H), 7.39-7.37 (m, 3H), 7.32 (brs, 2H), 4.46 (t, | | |
| | J = 6.4 Hz, 2H), 3.90 (t, J = 6.4 Hz, 2H), 3.54 (s, 3H), 1.74 (s, 6H). | | |
| A200 | NA | 508.1 | 530.0 (M + 23) |
| A201 | NA | 508.1 | 508.0 |
| A202 | NA | 522.1 | 522.0 |
| A203 | ¹H-NMR (400 MHz, CDCl₃) δ = 11.47-11.36 (m, 1H), 9.41 (s, 1H), 7.94 (d, J = 8.4 Hz, 2H), 7.50 | 545.1 | 545.1 |
| | (d, J = 2.4 Hz, 1H), 7.40 (d, J = 2.4 Hz, 1H), 7.38 (d, J = 8.4 Hz, 2H), 4.46 (t, J = 6.0 Hz, 2H), 3.90 (t, | | |
| | J = 6.4 Hz, 2H), 3.64 (s, 3H), 1.75 (s, 6H). | | |
| A204 | NA | 522.1 | 522.1 |
| A205 | NA | 524.1 | 524.2 |
| A206 | NA | 537.1 | 537.0 |
| A207 | NA | 508.1 | 508.1 |
| A208 | NA | 536.1 | 536.1 |
| A209 | NA | 550.1 | 550.0 |
| A210 | NA | 508.1 | 508.0 |
| A211 | ¹H-NMR (400 MHz, DMSO-d₆) δ = 8.20-8.16 (m, 1H), 8.06 (br s, 1H), 7.99 (br d, J = 7.2 Hz, 2H), | 544.1 | 544.0 |
| | 7.69 (d, J = 2.4 Hz, 1H), 7.62 (d, J = 2.0 Hz, 1H), 7.28 (d, J = 8.4 Hz, 2H), 4.42 (t, J = 5.2 Hz, 2H), | | |
| | 3.96 (t, J = 5.2 Hz, 2H), 3.09 (br s, 3H), 1.68 (s, 6H). | | |
| A214 | ¹H-NMR (400 MHz, CDCl₃) δ = 8.67 (s, 1H), 7.76 (br s, 1H), 7.52 (d, J = 8.0 Hz, 2H), 7.51 (d, | 558.1 | 558.0 |
| | J = 2.4 Hz, 1H), 7.37 (d, J = 2.4 Hz, 1H), 7.32 (d, J = 8.0 Hz, 2H), 4.69-4.68 (m, 1H), 4.46 (t, J = 6.0 | | |
| | Hz, 2H), 4.04-4.01 (m, 1H), 3.90 (t, J = 6.0 Hz, 2H), 3.50 (br dd, J = 17.2, 2.8 Hz, 1H), 3.09-3.02 | | |
| | (m, 5H), 2.30-2.26 (m, 1H), 2.04-1.99 (m, 1H), 1.73 (s, 6H). | | |
| A216 | ¹H-NMR (400 MHz, CDCl₃) δ = 8.64 (s, 1H), 7.68 (s, 1H), 7.52 (d, J = 8.4 Hz, 2H), 7.50 (d, J = 2.4 | 558.1 | 558.0 |
| | Hz, 1H), 7.36 (d, J = 2.4 Hz, 1H), 7.30 (d, J = 8.4 Hz, 2H), 5.82 (br s, 1H), 4.59 (br d, J = 2.4 Hz, | | |
| | 1H), 4.45 (t, J = 6.0 Hz, 2H), 3.89 (t, J = 6.0 Hz, 2H), 3.20 (s, 3H), 2.92 (br s, 2H), 2.60-2.45 (m, | | |
| | 1H), 2.11-1.97 (m, 2H), 1.96-1.88 (m, 1H), 1.72 (s, 6H). | | |
| A217 | ¹H-NMR (400 MHz, CDCl₃) δ = 8.65 (s, 1H), 7.80 (s, 1H), 7.53-7.50 (m, 3H), 7.36 (d, J = 2.0 Hz, | 544.1 | 544.0 |
| | 1H), 7.31 (s, 1H), 7.29 (s, 1H), 5.13 (br s, 1H), 5.01-4.96 (m, 1H), 4.45 (t, J = 6.0 Hz, 2H), 3.89 (t, | | |
| | J = 6.4 Hz, 2H), 3.24 (s, 3H), 3.08-3.04 (m, 1H), 2.98-2.94 (m, 1H), 2.85-2.82 (m, 1H), 2.20-2.14 | | |
| | (m, 1H), 1.72 (s, 6H). | | |
| A218 | ¹H-NMR (400 MHz, MeOD) δ = 9.50 (s, 1H), 8.55 (d, J = 5.2 Hz, 1H), 7.99 (s, 1H), 7.91 (d, J = 2.0 | 532.1 | 531.9 |
| | Hz, 1H), 7.78 (d, J = 8.8 Hz, 1H), 7.49 (s, 1H), 7.28 (d, J = 5.2 Hz, 1H), 7.07 (s, 1H), 6.95 (d, J = 8.4 | | |
| | Hz, 1H), 5.15 (s, 2H), 4.46 (t, J = 5.2 Hz, 2H), 3.95 (t, J = 5.2 Hz, 2H), 3.37 (s, 3H). | | |
| A219 | ¹H-NMR (400 MHz, CDCl₃) δ = 9.39 (s, 2H), 8.39 (s, 1H), 7.74 (d, J = 8.0 Hz, 2H), 7.51 (d, J = 2.0 | 556.1 | 556.0 |
| | Hz, 1H), 7.39-7.27 (m, 4H), 4.46 (t, J = 6.4 Hz, 2H), 3.89 (t, J = 6.0 Hz, 2H), 3.53 (s, 3H), 1.74 (s, | | |
| | 6H). | | |
| A220 | NA | 551.1 | 551.0 |
| A221 | NA | 534.1 | 534.0 |
| A222 | NA | 548.1 | 548.2 |
| A223 | NA | 534.1 | 534.0 |
| A224 | NA | 508.1 | 508.3 |
| A225 | NA | 508.1 | 508.0 |
| A226 | NA | 508.1 | 508.0 |
| A227 | NA | 508.1 | 508.0 |
| A228 | ¹H-NMR (400 MHz, DMSO-d₆) δ = 7.70 (d, J = 2.4 Hz, 1H), 7.68-7.61 (m, 2H), 7.58 (d, J = 8.4 Hz, | 545.1 | 562.0(M + 18) |
| | 2H), 7.31 (d, J = 8.4 Hz, 2H), 7.26-7.17 (m, 2H), 4.42 (t, J = 4.8 Hz, 2H), 4.1 (t, J = 4.4 Hz, 2H), 3.96 | | |
| | (t, J = 4.8 Hz, 2H), 3.84 (t, J = 4.4 Hz, 2H), 3.15 (s, 3H), 1.68 (s, 6H) | | |
| A229 | ¹H-NMR (400 MHz, CDCl₃) δ = 8.25 (s, 1H), 7.48 (br d, J = 8.4 Hz, 3H), 7.42 (s, 1H), 7.38 (s, | 546.1 | 546.0 |
| | 1H), 7.28 (br s, 2H), 4.44 (t, J = 6.0 Hz, 2H), 4.38-4.31 (m, 2H), 4.10-4.03 (m, 2H), 3.89 (t, J = 6.0 | | |
| | Hz, 2H), 3.58 (s, 3H), 1.71 (s, 6H) | | |
| A230 | ¹H-NMR (400 MHz, CDCl₃) δ = 7.48 (t, J = 8.8 Hz, 3H), 7.38 (d, J = 2.4 Hz, 1H), 7.22 (d, J = 8.4 Hz, | 575.1 | 575.0 |
| | 2H), 7.15-7.09 (m, 2H), 6.94 (d, J = 8.0 Hz, 1H), 4.76 (t, J = 6.0 Hz, 1H), 4.44 (t, J = 6.0 Hz, 2H), | | |
| | 4.41-4.30 (m, 2H), 4.12 (dd, J = 7.2, 11.6 Hz, 1H), 3.89 (t, J = 6.0 Hz, 2H), 3.58-3.51 (m, 1H), 3.48- | | |
| | 3.41 (m, 1H), 3.04 (s, 3H), 1.70 (s, 6H) | | |
| A233 | NA | 508.1 | 508.0 |
| A235 | ¹H-NMR (400 MHz, MeOD) δ = 7.99 (d, J = 2.4 Hz, 1H), 7.89 (d, J = 2.4 Hz, 1H), 7.69 (s, 1H), | 547.1 | 547.0 |
| | 7.57 (d, J = 8.4 Hz, 1H), 7.54 (d, J = 3.2 Hz, 1H), 7.30 (d, J = 8.4 Hz, 1H), 6.74 (d, J = 3.2 Hz, 1H), | | |
| | 4.55 (t, J = 5.6 Hz, 2H), 3.97 (t, J = 5.6 Hz, 4H), 3.69 (s, 4H), 2.93 (s, 3H), 2.80 (br s, 4H), 1.94 (s, | | |
| | 4H) | | |
| A236 | ¹H-NMR (400 MHz, CDCl₃) δ = 7.79 (d, J = 2.8 Hz, 1H), 7.67 (s, 1H), 7.65 (d, J = 2.8 Hz, 1H), 7.46 | 535.1 | 535.0 |
| | (d, J = 8.4 Hz, 1H), 7.31 (d, J = 8.4 Hz, 1H), 7.28 (s, 1H), 6.73 (d, J = 3.2 Hz, 1H), 5.04 (br s, 1H), | | |
| | 4.54 (t, J = 6.0 Hz, 2H), 3.96-3.93 (m, 4H), 3.22 (br d, J = 11.6 Hz, 2H), 3.04 (br s, 2H), 2.95 (s, | | |
| | 3H), 2.31 (brt, J = 11.2 Hz, 2H), 1.82 (br d, J = 12.0 Hz, 2H), 1.62-1.49 (m, 2H). | | |
| A237 | ¹H-NMR (400 MHz, MeOD) δ = 7.99 (d, J = 1.6 Hz, 1H), 7.88 (d, J = 1.6 Hz, 1H), 7.60 (s, 1H), | 535.1 | 535.0 |
| | 7.52 (d, J = 8.4 Hz, 1H), 7.49 (d, J = 3.2 Hz, 1H), 7.26 (d, J = 8.4 Hz, 1H), 6.70 (d, J = 3.2 Hz, 1H), | | |
| | 4.54 (t, J = 5.2 Hz, 2H), 3.97 (t, J = 5.6 Hz, 2H), 3.75-3.66 (m, 1H), 3.64 (s, 2H), 3.03 (br d, J = 12.0 | | |
| | Hz, 2H), 2.86 (s, 3H), 2.79 (s, 3H), 2.21-2.08 (m, 2H), 1.86 (dq, J = 3.6, 12.4 Hz, 2H), 1.74-1.62 | | |
| | (m, 2H). | | |
| A238 | ¹H-NMR (400 MHz, CDCl₃) δ = 7.79 (d, J = 2.4 Hz, 1H), 7.65 (t, J = 3.2 Hz, 2H), 7.45 (d, J = 8.4 Hz, | 535.1 | 535.1 |
| | 1H), 7.31 (br d, J = 8.4 Hz, 1H), 7.26 (br s, 1H), 6.71 (d, J = 3.2 Hz, 1H), 4.59 (br s, 1H), 4.54 (t, | | |
| | J = 6.0 Hz, 2H), 3.95 (t, J = 6.0 Hz, 2H), 3.85 (s, 2H), 3.05 (s, 3H), 2.79 (br s, 2H), 2.70 (t, J = 9.2 Hz | | |
| | 2H), 2.03 (d, J = 14.0 Hz, 2H), 1.93-1.83 (m, 2H), 1.49 (s, 3H). | | |
| A239 | ¹H-NMR (400 MHz, CDCl₃) δ = 7.80 (d, J = 2.8 Hz, 1H), 7.66 (d, J = 2.8 Hz, 1H), 7.60 (s, 1H), 7.46 | 557.1 | 557.0 |
| | (d, J = 8.4 Hz, 1H), 7.26-7.23 (m, 1H), 6.71 (d, J = 3.2 Hz, 1H), 4.54 (t, J = 6.0 Hz, 2H), 3.95 (t, | | |
| | J = 6.0 Hz, 2H), 3.85-3.74 (m, 1H), 3.72-3.59 (m, 2H), 3.30-3.15 (m, 1H), 3.06 (s, 3H), 2.98 (br d, | | |
| | J = 10.4 Hz, 1H), 2.45-2.19 (m, 2H), 2.15-2.01 (m, 1H), 1.92-1.74 (m, 1H). | | |
| A240 | ¹H-NMR (400 MHz, CDCl₃) δ = 9.66-9.36 (m, 1H), 8.66 (br d, J = 3.6 Hz, 1H), 7.29 (br s, 1H), | 536.2 | 536.2 |
| | 7.12 (br d, J = 8.8 Hz, 2H), 7.02 (s, 1H), 6.94-6.75 (m, 3H), 5.11 (s, 2H), 4.33 (br d, J = 2.0 Hz, 4H), | | |
| | 3.47 (s, 3H), 2.20 (br t, J = 7.6 Hz, 4H), 1.84 (quin, J = 7.6 Hz, 2H), 1.58 (s, 6H) | | |
| A241 | ¹H-NMR (400 MHz, MeOD) δ = 8.4 (s, 1H), 7.99 (d, J = 2.8 Hz, 1H), 7.89 (d, J = 2.4 Hz, 1H), 7.67 | 519.1 | 519.0 |
| | (s, 1H), 7.58 (d, J = 8.4 Hz, 1H), 7.55 (d, J = 3.2 Hz, 1H), 7.26 (d, J = 7.6 Hz, 1H), 6.75 (d, J = 3.2 Hz, | | |
| | 1H), 4.56 (t, J = 5.2 Hz, 2H), 4.05 (s, 6H), 3.98 (t, J = 5.2 Hz, 2H), 3.85 (s, 4H), 2.91 (s, 3H) | | |
| A242 | ¹H-NMR (400 MHz, CDCl₃) δ = 7.77 (d, J = 2.8 Hz, 1H), 7.64 (d, J = 2.4 Hz, 1H), 7.39 (d, J = 9.2 | 522.1 | 522.0 |
| | Hz, 1H), 7.23 (d, J = 3.2 Hz, 1H), 7.16 (d, J = 2.4 Hz, 1H), 6.93 (dd, J = 2.0, 8.8 Hz, 1H), 6.64 (d, | | |
| | J = 3.2 Hz, 1H), 4.53 (t, J = 6.0 Hz, 2H), 4.25-4.18 (m, 1H), 4.14 (br d, J = 7.6 Hz, 1H), 3.95 (t, J = 6.0 | | |
| | Hz, 2H), 3.50-3.40 (m, 1H), 3.01 (s, 3H), 2.19 (br d, J = 9.6 Hz, 4H), 1.68-1.62 (m, 2H), 1.48-1.40 | | |
| | (m, 2H) | | |
| A243 | ¹H-NMR (400 MHz, CDCl₃) δ = 8.70 (s, 1H), 8.03 (s, 1H), 7.53 (d, J = 8.4 Hz, 2H), 7.49 (d, J = 2.0 | 558.1 | 558.0 |
| | Hz, 1H), 7.39 (d, J = 2.4 Hz, 1H), 7.29 (br d, J = 8.4 Hz, 2H), 4.85-4.72 (m, 1H), 4.55 (br d, J = 8.8 | | |
| | Hz, 1H), 4.44 (t, J = 6.0 Hz, 2H), 3.89 (t, J = 6.0 Hz, 2H), 3.12 (s, 3H), 3.08-2.96 (m, 2H), 2.29-2.19 | | |
| | (m, 1H), 2.13-1.90 (m, 3H), 1.71 (s, 6H). | | |
| A244 | ¹H-NMR (400 MHz, CDCl₃) δ = 7.78 (d, J = 2.4, 1H), 7.65 (d, J = 2.4 Hz, 1H), 7.51 (s, 1H), 7.42 (d, | 521.1 | 521.3 |
| | J = 8.4 Hz, 1H), 7.24 (d, J = 3.2 Hz, 1H), 7.13 (d, J = 8.4 Hz, 1H), 6.68 (d, J = 3.2 Hz, 1H), 5.60-5.11 | | |
| | (m, 1H), 4.53 (t, J = 6.0 Hz, 2H), 3.95 (t, J = 6.0 Hz, 2H), 3.58-3.41 (m, 4H), 3.04 (s, 3H), 2.99-2.92 | | |
| | (m, 2H), 2.91-2.83 (m, 2H), 1.72 (s, 3H). | | |
| A245 | ¹H-NMR (400 MHz, CDCl₃) δ = 7.81 (d, J = 2.4 Hz, 1H), 7.67 (d, J = 2.4 Hz, 1H), 7.50 (s, 1H), 7.43 | 535.1 | 535.0 |
| | (d, J = 8.4 Hz, 1H), 7.24 (d, J = 3.2 Hz, 1H), 7.12 (d, J = 8.8 Hz, 1H), 6.68 (d, J = 3.2 Hz, 1H), 5.47- | | |
| | 5.32 (m, 1H), 4.53 (t, J = 6.0 Hz, 2H), 3.95 (t, J = 6.0 Hz, 2H), 3.71 (s, 1H), 2.98-2.94 (m, 2H), 2.91 | | |
| | (s, 3H), 2.98-2.73 (m, 5H), 2.46-2.36 (m, 1H) 1.97-1.90 (m, 4H). | | |
| A246 | ¹H-NMR (400 MHz, CDCl₃) δ = 7.41 (d, J = 2.0 Hz, 1H), 7.27 (s, 1H), 6.82 (d, J = 8.4 Hz, 2H), 6.71 | 521.1 | 543.3 (M + 23) |
| | (dd, J = 2.0, 8.0 Hz, 1H), 4.91 (s, 1H), 4.43 (t, J = 6.0 Hz, 2H), 4.27 (t, J = 8.0 Hz, 1H), 4.09 (s, 2H), | | |
| | 3.89 (t, J = 6.0 Hz, 2H), 3.01 (s, 3H), 3.00-2.83 (m, 2H), 2.67-2.55 (m, 1H), 2.13 (s, 6H), 2.02-1.91 | | |
| | (m, 1H) | | |
| A247 | ¹H-NMR (400 MHz, CDCl₃) δ = 7.34 (d, J = 2.4 Hz, 1H), 7.16 (d, J = 2.0 Hz, 1H), 6.69-6.63 (m, | 535.1 | 557.0 (M + 23) |
| | 3H), 4.88 (s, 1H), 4.43 (t, J = 6.0 Hz, 2H), 4.07 (s, 3H), 3.89 (t, J = 6.0 Hz, 2H), 3.01 (s, 3H), 2.89- | | |
| | 2.78 (m, 2H), 2.18-2.16 (m, 1H), 2.13 (s, 6H), 1.79-1.74 (m, 3H). | | |
| A248 | ¹H-NMR (400 MHz, MeOD) δ = 7.98 (d, J = 2.8 Hz, 1H), 7.87 (d, J = 2.8 Hz, 1H), 7.48 (s, 1H), | 521.1 | 521.3 |
| | 7.46 (s, 1H), 7.45 (d, J = 3.2 Hz, 1H), 7.15-7.10 (m, 1H), 6.65 (d, J = 3.2 Hz, 1H), 4.53 (t, J = 5.6 Hz, | | |
| | 2H), 3.97 (t, J = 5.6 Hz, 2H), 3.45-3.34 (m, 2H), 3.18-3.07 (m, 2H), 3.02-2.90 (m, 2H), 2.86 (s, | | |
| | 3H), 2.82-2.75 (m, 2H), 2.72-2.64 (m, 1H), 2.15-2.15 (m, 1H), 2.15-2.07 (m, 1H), 2.04-1.94 (m, | | |
| | 1H) | | |
| A249 | 1H-NMR (400 MHz, CDCl₃) δ = 7.78 (d, J = 2.4 Hz, 1H), 7.64 (d, J = 2.4 Hz, 1H), 7.51 (s, 1H), | 533.1 | 533.0 |
| | 7.42 (d, J = 8.4 Hz, 1H), 7.23 (d, J = 3.2 Hz, 1H), 7.13 (dd, J = 1.2, 8.4 Hz, 1H), 6.68 (d, J = 3.2 Hz, | | |
| | 1H), 4.53 (t, J = 6.0 Hz, 2H), 3.95 (t, J = 6.0 Hz, 2H), 3.89-3.79 (m, 6H), 3.04-2.97 (m, 2H), 2.95- | | |
| | 2.83 (m, 5H), 2.61 (t, J = 7.2 Hz, 2H). | | |
| A251 | ¹H-NMR (400 MHz, CDCl₃) δ = 8.99 (s, 1H), 8.65 (d, J = 5.2 Hz, 1H), 7.30 (d, J = 5.2 Hz, 1H), 7.14 | 496.2 | 496.0 |
| | (d, J = 8.8 Hz, 2H), 7.05 (s, 1H), 6.88 (d, J = 8.8 Hz, 2H), 6.87-6.81 (m, 1H), 5.11 (s, 2H), 4.41 (dt, | | |
| | J = 2.8, 7.2 Hz, 4H), 3.48 (s, 3H), 2.37 (q, J = 7.6, 7.6, 7.2, 7.6 Hz, 2H), 1.60 (s, 6H) | | |
| A252 | ¹H-NMR (400 MHz, CDCl₃) δ = 8.62 (d, J = 5.2 Hz, 1H), 7.29 (d, J = 5.2 Hz, 1H), 7.12 (dd, J = 2.0, | 510.2 | 510.3 |
| | 6.8 Hz, 2H), 7.03 (dd, J = 0.8, 2.0 Hz, 1H), 6.87 (dd, J = 2.0, 6.8 Hz, 2H), 6.83 (dd, J = 2.4, 15.2 Hz, | | |
| | 1H), 5.09 (s, 2H), 4.51 (dt, J = 8.0, 8.0 Hz, 2H), 3.95 (ddd, J = 2.8, 5.6, 8.4 Hz, 2H), 3.47 (s, 3H), | | |
| | 2.86-2.71 (m, 1H), 1.58 (s, 6H), 1.28 (d, J = 6.4 Hz, 3H) | | |
| A253 | ¹H-NMR (400 MHz, CDCl₃) δ = 8.72(s, 1H), 8.63 (d, J = 4.8 Hz, 1H), 7.28 (d, J = 5.2 Hz, 1H), 7.12 | 526.2 | 526.0 |
| | (dd, J = 2.4, 9.2 Hz, 2H), 7.04 (dd, J = 1.2, 2.4 Hz, 1H), 6.87 (dd, J = 2.4, 10.4 Hz, 2H), 6.82 (d, | | |
| | J = 2.4 Hz, 1H), 5.10 (s, 2H), 4.46 (dt, J = 8.4, 8.4 Hz, 2H), 4.19 (ddd, J = 2.8, 5.2, 8.4 Hz, 2H), 3.87 | | |
| | (d, J = 6.4 Hz, 2H), 3.47 (s, 3H), 2.97-2.78 (m, 1H), 1.58 (s, 6H) | | |
| A254 | ¹H-NMR (400 MHz, MeOD) δ = 7.63 (s, 1H), 7.59 (d, J = 8.4 Hz, 2H), 7.58-7.54 (m, 2H), 7.51 | 544.1 | 544.0 |
| | (dd, J = 1.2, 8.4 Hz, 1H), 7.37-7.30 (m, 3H), 4.43 (t, J = 5.6 Hz, 2H), 3.90 (t, J = 5.6 Hz, 2H), 3.12 (s, | | |
| | 3H), 1.73 (s, 6H). | | |
| A255 | ¹H-NMR (400 MHz, MeOD) δ = 7.59 (s, 1H), 7.58-7.56 (m, 2H), 7.54 (d, J = 2.4 Hz, 1H), 7.52 (s, | 544.1 | 544.0 |
| | 1H), 7.42 (d, J = 2.8 Hz, 2H), 7.34 (s, 1H), 7.32 (s, 1H), 4.43 (t, J = 5.6 Hz, 2H), 3.90 (t, J = 5.6 Hz, | | |
| | 2H), 3.12 (s, 3H), 1.73 (s, 6H) | | |
| A256 | ¹H-NMR (400 MHz, CDCl₃) δ = 8.35 (s, 1H), 7.66 (d, J = 1.6 Hz, 1H), 7.50 (dd, J = 2.4, 5.2 Hz, | 545.1 | 544.9 |
| | 3H), 7.36 (d, J = 2.0 Hz, 1H), 7.33 (br d, J = 8.0 Hz, 3H), 4.45 (t, J = 6.4 Hz, 2H), 3.89 (t, J = 6.0 Hz, | | |
| | 2H), 3.20 (s, 3H), 1.72 (s, 6H) | | |
| A257 | ¹H-NMR (400 MHz, DMSO-d₆) δ = 8.43 (d, J = 1.6 Hz, 1H), 8.22 (d, J = 1.6 Hz, 1H), 7.71-7.65 (m, | 545.1 | 545.0 |
| | 4H), 7.37 (d, J = 8.4 Hz, 2H), 4.43 (t, J = 5.2 Hz, 2H), 3.96 (t, J = 4.8 Hz, 2H), 3.07 (s, 3H), 1.70 (s, | | |
| | 6H). | | |
| A258 | ¹H-NMR (400 MHz, CDCl₃) δ = 8.65 (d, J = 4.0 Hz, 1H), 7.45 (d, J = 2.0 Hz, 1H), 7.32 (d, J = 2.0 | 565.1 | 565.0 |
| | Hz, 1H), 7.29 (d, J = 4.4 Hz, 1H), 7.13 (br d, J = 8.8 Hz, 2H), 6.91 (br d, J = 8.4 Hz, 2H), 5.11 (s, | | |
| | 2H), 4.36-4.31 (m, 2H), 4.30-4.24 (m, 1H), 3.83 (dq, J = 5.6, 11.2 Hz, 2H), 3.48 (s, 3H), 1.65 (s, | | |
| | 6H) | | |
| A259 | ¹H-NMR (400 MHz, CDCl₃) δ = 8.97 (s, 1H), 8.64 (d, J = 5.2 Hz, 1H), 7.29 (d, J = 5.2 Hz, 1H), 7.12 | 512.2 | 512.1 |
| | (d, J = 8.8 Hz, 2H), 7.05 (s, 1H), 6.91-6.83 (m, 3H), 5.10 (s, 2H), 4.72 (s, 1H), 4.69-4.62 (m, 2H), | | |
| | 4.26-4.13 (m, 2H), 3.47 (s, 3H), 2.10 (s, 1H), 1.59 (s, 6H) | | |
| A260 | ¹H-NMR (400 MHz, CDCl₃) δ = 8.60 (d, J = 4.8 Hz, 1H), 8.09 (s, 1H), 7.28 (br s, 1H), 7.12 (d, | 524.2 | 524.2 |
| | J = 8.8 Hz, 2H), 7.02 (s, 1H), 6.87 (d, J = 8.4 Hz, 2H), 6.82 (dd, J = 2.0, 15.2 Hz, 1H), 5.08 (s, 2H), | | |
| | 4.08 (d, J = 3.2 Hz, 4H), 3.47 (s, 3H), 1.58 (s, 6H), 1.32 (s, 6H) | | |
| A261 | ¹H-NMR (400 MHz, MeOD) δ = 8.54 (d, J = 5.2 Hz, 1H), 7.24 (d, J = 2.4 Hz, 1H), 7.23 (d, J = 5.2 | 524.2 | 524.1 |
| | Hz, 1H), 7.17 (dd, J = 2.0, 6.8 Hz, 2H), 7.12 (dd, J = 2.0, 14.0 Hz, 1H), 6.98-6.91 (dd, J = 2.0, 6.8 | | |
| | Hz, 2H), 5.13 (s, 2H), 3.37 (s, 3H), 3.22-3.18 (m, 4H), 1.74-1.68 (m, 4H), 1.63 (s, 8H) | | |
| A262 | ¹H-NMR (400 MHz, CDCl₃) δ = 8.91 (s, 1H), 8.64 (d, J = 5.2 Hz, 1H), 7.29 (d, J = 5.2 Hz, 1H), | 526.2 | 526.2 |
| | 7.211 (m, 1H), 7.13 (dd, J = 2.4, 6.8 Hz, 2H), 7.07 (dd, J = 2.4, 14.4 Hz, 1H), 6.90 (dd, J = 2.4, 6.8 | | |
| | Hz, 2H), 5.11 (s, 2H), 3.85 (t, J = 4.4 Hz 4H), 3.47 (s, 3H), 3.29 (m, 4H), 1.63 (s, 6H) | | |
| A263 | ¹H-NMR (400 MHz, CDCl₃) δ = 8.87 (s, 1H), 8.75 (br s, 1H), 8.04 (s, 1H), 7.59 (d, J = 8.4 Hz, | 544.1 | 544.0 |
| | 2H), 7.50 (d, J = 2.0 Hz, 1H), 7.38 (d, J = 2.0 Hz, 1H), 7.26 (d, J = 7.2 Hz, 2H), 7.12 (s, 1H), 4.45 (t, | | |
| | J = 6.0 Hz, 2H), 3.89 (t, J = 6.0 Hz, 2H), 3.51 (s, 3H), 1.71 (s, 6H). | | |
| A264 | ¹H-NMR (400 MHz, CDCl₃) δ = 7.46 (d, J = 2.0 Hz, 1H), 7.30 (d, J = 2.0 Hz, 1H), 7.06 (d, J = 8.8 | 537.1 | 559.0 |
| | Hz, 2H), 6.81 (d, J = 8.4 Hz, 2H), 4.97 (s, 1H), 4.52 (q, J = 6.0 Hz, 1H), 4.42 (t, J = 6.4 Hz, 2H), 3.88 | | |
| | (t, J = 6.0 Hz, 2H), 2.99 (s, 3H), 1.98-2.11 (m, 6H), 1.64 (s, 6H), 1.28 (d, J = 6.0 Hz, 3H) | | |
| A265 | ¹H-NMR (400 MHz, CDCl₃) δ = 7.46 (d, J = 2.4 Hz, 1H), 7.30 (d, J = 2.4 Hz, 1H), 7.07 (d, J = 8.8 | 537.1 | 559 (M + 23) |
| | Hz, 2H), 6.81 (d, J = 8.8 Hz, 2H), 4.84 (br s, 1H), 4.51 (q, J = 6.0 Hz, 1H), 4.42 (t, J = 6.4 Hz, 2H), | | |
| | 3.88 (t, J = 6.0 Hz, 2H), 2.99 (s, 3H), 2.07-2.02 (m, 6H), 1.64 (s, 6H), 1.28 (d, J = 6.0 Hz, 3H) | | |
| A266 | ¹H-NMR (400 MHz, CDCl₃) δ = 7.43 (d, J = 2.4 Hz, 1H), 7.28 (d, J = 2.0 Hz, 1H), 6.87 (d, J = 2.0 | 509.1 | 509.2 |
| | Hz, 1H), 6.84 (d, J = 8.4 Hz, 1H), 6.78-6.71 (m, 1H), 4.51 (br d, J = 8.8 Hz, 1H), 4.44 (t, J = 6.0 Hz, | | |
| | 2H), 4.28 (t, J = 8.0 Hz, 1H), 4.18 (sxt, J = 8.4 Hz, 1H), 4.00 (d, J = 6.4 Hz, 2H), 3.89 (t, J = 6.0 Hz, | | |
| | 2H), 3.09-2.87 (m, 5H), 2.75-2.56 (m, 2H), 2.45 (tdd, J = 3.2, 7.8, 10.6 Hz, 2H), 2.29-2.16 (m, | | |
| | 2H), 2.00-1.91 (m, 1H) | | |
| A268 | ¹H-NMR (400 MHz, CDCl₃) δ = 7.42 (d, J = 2.4 Hz, 1H), 7.30 (d, J = 2.4 Hz, 1H), 7.09 (dd, J = 2.0, | 475.1 | 475.0 |
| | 6.8 Hz, 2H), 6.8 (dd, J = 2.4, 6.8 Hz, 2H), 4.96 (s, 1H), 4.08 (s, 2H), 4.05 (s, 3H), 3.01 (s, 3H), | | |
| | 2.12 (s, 6H), 1.64 (s, 6H) | | |
| A269 | ¹H-NMR (400 MHz, CDCl₃) δ = 8.44 (br s, 1H), 8.17 (d, J = 1.2 Hz, 1H), 7.91 (br d, J = 8.4 Hz, | 507.1 | 507.1 |
| | 1H), 7.63 (d, J = 8.4 Hz, 2H), 7.51-7.60 (m, 1H), 7.49 (d, J = 2.4 Hz, 1H), 7.38 (d, J = 2.4 Hz, 1H), | | |
| | 7.32 (d, J = 8.4 Hz, 2H), 4.07 (s, 3H), 3.27 (br s, 3H), 1.73 (s, 6H). | | |
| A272 | ¹H-NMR (400 MHz, CDCl₃) δ = 7.44 (d, J = 2.0 Hz, 1H), 7.32 (d, J = 2.4 Hz, 1H), 7.09 (d, J = 8.8 | 551.1 | 551.3 |
| | Hz, 2H), 6.84 (d, J = 8.8 Hz, 2H), 4.59 (s, 1H), 4.42 (t, J = 6.0 Hz, 2H), 3.94 (s, 2H), 3.88 (t, J = 6.0 | | |
| | Hz, 2H), 3.04 (s, 3H), 2.04-1.97 (m, 2H), 1.89-1.84 (m, 4H), 1.64 (s, 6H), 1.56 (s, 4H) | | |
| A273 | ¹H-NMR (400 MHz, CDCl₃) δ = 7.44 (d, J = 2.4 Hz, 1H), 7.31 (d, J = 2.4 Hz, 1H), 7.07 (d, J = 6.8 | 565.2 | 565.3 |
| | Hz, 2H), 6.81 (d, J = 6.8 Hz, 2H), 4.42 (t, J = 6.0 Hz, 2H), 4.10 (s, 1H), 3.88 (t, J = 6.4 Hz, 2H), 3.56 | | |
| | (s, 2H), 3.03 (s, 3H), 1.96-1.92 (m, 6H), 1.72-1.68 (m, 6H), 1.64 (s, 6H) | | |
| A274 | ¹H-NMR (400 MHz, CDCl₃) δ = 7.79 (d, J = 8.8 Hz, 1H), 7.75 (d, J = 1.2 Hz, 1H), 7.64 (dd, J = 1.6, | 560.1 | 560.2 |
| | 8.4 Hz, 1H), 7.54-7.50 (m, 3H), 7.37 (d, J = 2.4 Hz, 1H), 7.29-7.27 (m, 2H), 4.44 (t, J = 6.0 Hz, | | |
| | 2H), 3.89 (t, J = 6.0 Hz, 2H), 3.14 (s, 3H), 1.72 (s, 6H). | | |
| A275 | ¹H-NMR (400 MHz, DMSO-d₆) δ = 7.84 (d, J = 8.0 Hz, 1H), 7.71 (d, J = 2.4 Hz, 1H), 7.65 (d, | 560.1 | 560.0 |
| | J = 2.4 Hz, 1H), 7.61 (d, J = 8.4 Hz, 2H), 7.52 (d, J = 8.0 Hz, 1H), 7.49 (s, 1H), 7.36 (d, J = 8.4 Hz, | | |
| | 2H), 4.43 (t, J = 5.2 Hz, 2H), 3.96 (t, J = 5.2 Hz, 2H), 3.01 (s, 3H), 1.70 (s, 6H) | | |
| A277 | ¹H NMR (400 MHz, CDCl₃) δ = 7.44 (d, J = 2.4 Hz, 1H), 7.30 (d, J = 2.0 Hz, 1H), 7.08 (d, J = 6.8 | 537.1 | 537.0 |
| | Hz, 2H), 6.83 (d, J = 6.8 Hz, 2H), 4.42 (t, J = 6.4 Hz, 2H), 4.20-4.19 (m, 1H), 3.99 (s, 2H), 3.88 (t, | | |
| | J = 6.4 Hz, 2H), 3.26 (d, J = 6.0 Hz, 2H), 2.97 (s, 3H), 1.78 (s, 6H), 1.64 (s, 6H) | | |
| A278 | ¹H-NMR (400 MHz, CDCl₃) δ = 8.84 (d, J = 8.8 Hz, 1H), 8.11 (d, J = 8.0 Hz, 2H), 7.91 (d, J = 8.4 | 555.1 | 555.3 |
| | Hz, 1H), 7.47 (d, J = 2.0 Hz, 1H), 7.39-7.36 (m, 4H), 7.09 (d, J = 7.2 Hz, 1H), 4.45 (t, J = 6.0 Hz, | | |
| | 2H), 3.89 (t, J = 6.0 Hz, 2H), 3.18 (s, 3H), 1.74 (s, 6H). | | |
| A282 | ¹H-NMR (400 MHz, DMSO-d₆) δ = 7.95 (d, J = 8.4 Hz, 2H), 7.79 (d, J = 8.0 Hz, 2H), 7.70 (d, | 545.1 | 545.0 |
| | J = 2.0 Hz, 1H), 7.63-7.61 (m, 2H), 7.35 (d, J = 8.4 Hz, 1H), 4.42 (t, J = 4.8 Hz, 2H), 3.96 (t, J = 5.2 | | |
| | Hz, 2H), 3.05 (s, 3H), 1.69 (s, 6H) | | |
| A283 | ¹H-NMR (400 MHz, CDCl₃) δ = 9.32 (br s, 1H), 8.66 (d, J = 5.2 Hz, 1H), 7.38 (d, J = 2.0 Hz, 1H), | 542.2 | 542.0 |
| | 7.31 (d, J = 2.0 Hz, 1H), 7.29 (d, J = 5.2 Hz, 1H), 7.13 (d, J = 8.8 Hz, 2H), 6.90 (d, J = 9.2 Hz, 2H), | | |
| | 5.12 (s, 2H), 3.87-3.85 (m, 4H), 3.47 (s, 3H), 3.35-3.33 (m, 4H), 1.64 (s, 6H) | | |
| A285 | ¹H-NMR (400 MHz, CDCl₃) δ = 7.44 (d, J = 2.4 Hz, 1 H), 7.31 (d, J = 2.4 Hz, 1 H), 7.10 (d, J = 8.8 | 523.1 | 523.0 |
| | Hz, 2 H), 6.83 (d, J = 8.8 Hz, 2 H), 4.42 (t, J = 6.0 Hz, 2 H), 4.31 (s, 1 H), 4.16 (s, 2 H), 3.88 (t, | | |
| | J = 6.0 Hz, 2 H), 3.46 (s, 2 H), 2.97 (s, 3 H), 1.99 (br d, J = 4.8 Hz, 2 H), 1.77 (dd, J = 4.8, 1.6 Hz, 2 | | |
| | H), 1.65 (s, 6H) | | |

Biological Assays

### Example 26: Activity of Exemplary Compounds in Cellular Assays

The PSA (6.1 kb)-luciferase reporter contains functional AREs (androgen response elements) to which AR binds in response to androgen to induce luciferase activity. LNCaP cells were transiently transfected with the PSA (6.1 kb)-luciferase reporter for 24 h, and then pretreated with vehicle (DMSO) or indicated concentration of representative compounds for one hour prior to the addition of synthetic androgen, R1881 (1 nM). After 24 h of incubation with R1881, the cells were harvested, and relative luciferase activities were determined (FIG. 1). To determine the IC₅₀, treatments were normalized to vehicle control activity induced by R1881 (Table 2).

Luciferase Assay: Cells were lysed using Passive Lysis Buffer (Promega) and then collected into V-bottom 96-well tissue culture plates. Lysates were centrifuged at 4° C. for 5 minutes at 3000 rpm. To measure luminescence of LNCaP cell lysates the Firefly Luciferase Assay System (Promega) was employed, according to manufacturer's protocol. Relative luminescence units (RLU) in cell lysates were detected for 10 seconds using Promega GloMax-Multi Detection Luminometer (Promega). Values were normalized to protein content. GraphPad Prism graphing software was used to calculate IC₅₀ values.

Statistical analyses were performed using GraphPad Prism (Version 6.01 for Windows; La Jolla, Calif., USA). Comparisons between treatment and control groups were compared using Two-Way ANOVA with post-hoc Dunnett's and Tukey's tests. Differences were considered statistically significant at P values less than 0.05.

Table 2 shows IC₅₀ ranges of Compounds from Table A.

TABLE 2
IC₅₀ Ranges of Various Compounds in
Androgen-Induced PSA-Luciferase Assay

| Compound ID | PSA-Luc IC50 Range |
|---|---|
| A1 | * |
| A2 | * |
| A3 | |
| A4 | |
| A5 | * |
| A6 | * |
| A7 | * |
| A8 | * |
| A9 | |
| A10 | * |
| A11 | |
| A12 | |
| A13 | |
| A14 | |
| A15 | |
| A16 | |
| A17 | |
| A18 | * |
| A19 | |
| A20 | ** |
| A21 | ** |
| A22 | * |
| A23 | |
| A24 | |
| A25 | ** |
| A26 | * |
| A27 | ** |
| A28 | ** |
| A29 | ** |
| A30 | * |
| A31 | * |
| A32 | ** |
| A33 | ** |
| A34 | ** |
| A35 | ** |
| A35(R) | *** |
| A35(S) | ** |
| A36 | *** |
| A37 | ** |
| A38 | * |
| A39 | |
| A40 | * |
| A41 | * |
| A42 | |
| A43 | *** |
| A44 | ** |
| A45 | *** |
| A46 | *** |
| A47 | ** |
| A48 | ** |
| A49 | ** |
| A50 | ** |
| A51 | *** |
| A52 | ** |
| A53 | |
| A54 | ** |
| A55 | * |
| A56 | ** |
| A57 | *** |
| A58 | * |
| A59 | *** |
| A60 | * |
| A61 | *** |
| A62 | |
| A63 | |
| A64 | * |
| A65 | |
| A66 | ** |
| A67 | * |
| A68 | |
| A69 | |
| A70 | ** |
| A71 | * |
| A72 | * |
| A73 | * |
| A74 | ** |
| A75 | * |
| A76 | *** |
| A77 | * |
| A78 | * |
| A79 | |
| A80 | |
| A81 | * |
| A82 | |
| A83 | |
| A84 | * |
| A85 | * |
| A86 | |
| A87 | |
| A88 | |
| A89 | * |
| A90 | ** |
| A91 | ** |
| A92 | |
| A93 | ** |
| A94 | ** |
| A95 | *** |
| A96 | |
| A97 | *** |
| A98 | ** |
| A99 | ** |
| A100 | *** |
| A101 | |
| A102 | ** |
| A103 | * |
| A104 | * |
| A105 | * |
| A106 | * |
| A107 | *** |
| A108 | * |
| A109 | |
| A110 | * |
| A111 | * |
| A112 | * |
| A113 | ** |
| A114 | ** |
| A115 | * |
| A116 | ** |
| A117 | ** |
| A118 | |
| A119 | |
| A120 | *** |
| A121 | * |
| A122 | * |
| A123 | * |
| A124 | ** |
| A125 | * |
| A126 | ** |
| A127 | ** |
| A128 | ** |
| A129 | |
| A130 | |
| A131 | |
| A132 | ** |
| A133 | * |
| A134 | * |
| A135 | ** |
| A136 | ** |
| A137 | ** |
| A138 | * |
| A139 | * |
| A140 | |
| A141 | * |
| A142 | * |
| A143 | * |
| A144 | ** |
| A145 | ** |
| A146 | ** |
| A147 | |
| A148 | * |
| A149 | |
| A150 | *** |
| A151 | ** |
| A152 | * |
| A153 | * |
| A154 | * |
| A155 | ** |
| A156 | |
| A157 | ** |
| A158 | ** |
| A159 | ** |
| A160 | * |
| A161 | * |
| A162 | ** |
| A163 | ** |
| A164 | * |
| A165 | |
| A166 | * |
| A167 | ** |
| A168 | *** |
| A169 | * |
| A170 | * |
| A171 | * |
| A172 | |
| A173 | *** |
| A174 | * |
| A175 | |
| A176 | * |
| A177 | ** |
| A178 | |
| A179 | |
| A180 | ** |
| A181 | ** |
| A182 | |
| A183 | |
| A184 | * |
| A185 | |
| A186 | |
| A187 | ** |
| A188 | |
| A189 | ** |
| A190 | * |
| A191 | *** |
| A192 | ** |
| A193 | |
| A194 | *** |
| A195 | * |
| A196 | ** |
| A197 | * |
| A198 | ** |
| A199 | *** |
| A200 | ** |
| A201 | * |
| A202 | * |
| A203 | ** |
| A204 | * |
| A205 | * |
| A206 | ** |
| A207 | ** |
| A208 | * |
| A209 | ** |
| A210 | ** |
| A211 | * |
| A212 | |
| A213 | |
| A214 | ** |
| A215 | |
| A216 | * |
| A217 | ** |
| A218 | * |
| A219 | ** |
| A220 | * |
| A221 | ** |
| A222 | ** |
| A223 | ** |
| A224 | ** |
| A225 | ** |
| A226 | ** |
| A227 | ** |
| A228 | * |
| A229 | ** |
| A230 | * |
| A231 | |
| A232 | |
| A233 | ** |
| A234 | |
| A235 | * |
| A236 | * |
| A237 | * |
| A238 | * |
| A239 | ** |
| A240 | * |
| A241 | * |
| A242 | ** |
| A243 | * |
| A244 | * |
| A245 | * |
| A246 | ** |
| A247 | * |
| A248 | * |
| A249 | * |
| A250 | |
| A251 | ** |
| A252 | ** |
| A253 | * |
| A254 | ** |
| A255 | ** |
| A256 | * |
| A257 | * |
| A258 | * |
| A259 | *** |
| A260 | ** |
| A261 | ** |
| A262 | ** |
| A263 | * |
| A264 | ** |
| A265 | ** |
| A266 | ** |
| A267 | ** |
| A268 | ** |
| A269 | * |
| A270 | ** |
| A271 | ** |
| A272 | ** |
| A273 | ** |
| A274 | *** |
| A275 | ** |
| A276 | ** |
| A277 | *** |
| A278 | ** |
| A279 | ** |
| A280 | ** |
| A281 | * |
| A282 | * |
| A283 | ** |
| A284 | ** |
| A285 | ** |

Note:
*** represent IC₅₀ <500 nM,
** represents IC₅₀ in the range of 500-2000 nM,
* represents IC₅₀ in the range of >2000 nM

### Example 27: Stability Assays

Microsomal Stability Assay: Microsomal stability assay is a widely used in vitro model to characterize the metabolic conversion by phase I enzymes, such as cytochrome P450 (CYP) enzymes. Since metabolism is known to be highly variable in different species, microsomal stability assay is commonly run in multiple species. Metabolic stability of testing compound can be evaluated using human, rat, mouse, or other animal liver or intestine microsomes to predict intrinsic clearance.

The assay was carried out in 96-well microtiter plates at 37° C. Reaction mixtures (25 μL) contained a final concentration of 1 μM test compound, 0.5 mg/mL liver microsomes protein, and 1 mM NADPH and/or 1 mM UDPGA (with alamethicin) in 100 mM potassium phosphate, pH 7.4 buffer with 3 mM MgCl₂. The incubation was done with N=1, but duplicate incubation at each time point can be prepared if necessary. At each of the time points (for example, 0, 15, 30, and 60 minutes), 150 μL of quench solution (100% acetonitrile with 0.1% formic acid) with internal standard was transferred to each well. Besides the zero minute controls, mixtures containing the same components except the NADPH can also be prepared as the negative control. Verapamil was included as a positive control to verify assay performance. Plates were sealed, vortexed, and centrifuged at 4° C. for 15 minutes at 4000 rpm. The supernatant is transferred to fresh plates for LC/MS/MS analysis.

Summarized conditions: [Compound]=1 μM, [LM]=0.5 mg/mL, [NADPH]=1 mM and/or [UDPGA]=1 mM, Buffer=100 mM Potassium Phosphate, pH 7.4 with 3 mM MgCl₂, Time=0, 15, 30, and 60 min, and Temperature=37° C.

All samples were analyzed on LC/MS/MS using an AB Sciex API 4000 instrument, coupled to a Shimadzu LC-20AD LC Pump system. Analytical samples were separated using a Waters Atlantis T3 dC18 reverse phase HPLC column (20 mm×2.1 mm) at a flow rate of 0.5 mL/min. The mobile phase consisted of 0.1% formic acid in water (solvent A) and 0.1% formic acid in 100% acetonitrile (solvent B).

The extent of metabolism is calculated as the disappearance of the test compound, compared to the 0-min time incubation. Initial rates are calculated for the compound concentration and used to determine half-life (t½).

Liver microsome half life of representative compounds are shown in Tables 2. Compound X has the following structures:

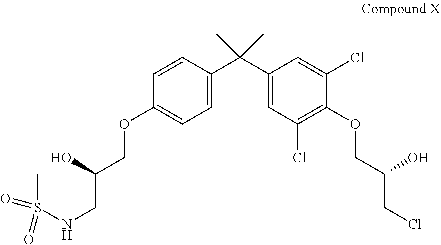 [SMILES: CC(C)(c1ccc(OC[C@H](O)CNS(C)(=O)=O)cc1)c1cc(Cl)c(OC[C@H](O)CCl)c(Cl)c1]

TABLE 3
Liver Microsome Half-Life

| | Liver microsome T_{1/2} (min) | | | | |
|---|---|---|---|---|---|
| Compound ID | Human | Mouse | Rat | Dog | Monkey |
| A5 | 55.8 | NA | NA | NA | NA |
| A6 | >120 | NA | NA | NA | NA |
| A7 | 84.28 | NA | NA | NA | NA |
| A8 | 9.67 | NA | NA | NA | NA |
| A32 | 68.34 | >120 | NA | NA | NA |
| A33 | >120 | >120 | NA | NA | NA |
| A34 | >120 | >120 | NA | NA | NA |
| A35 | >120 | >120 | >120 | >120 | >120 |
| A36 | >120 | >120 | >120 | >120 | >120 |
| A37 | >120 | NA | NA | NA | NA |
| A38 | >120 | NA | NA | NA | NA |
| A41 | >120 | >120 | NA | NA | NA |
| A48 | >120 | >120 | >120 | >120 | >120 |
| A51 | >120 | >120 | >120 | 107 | >120 |
| A57 | 69 | >120 | >120 | >120 | 68 |
| A59 | 11 | >120 | >120 | 58 | 7 |
| A100 | >120 | 96 | 51 | >120 | 38 |
| A107 | >120 | >120 | >120 | >120 | >120 |
| A113 | 116 | >120 | >120 | >120 | >120 |
| A114 | >120 | >120 | >120 | 63 | >120 |
| A116 | >120 | >120 | >120 | >120 | >120 |
| A126 | >120 | >120 | 57 | 44 | 35 |
| A132 | >120 | >120 | 97 | >120 | 57 |
| A135 | >120 | 118 | >120 | >120 | 44 |
| A141 | >120 | >120 | >120 | >120 | 66 |
| A144 | >120 | >120 | >120 | >120 | >120 |
| A150 | >120 | >120 | >120 | >120 | >120 |
| A169 | >120 | >120 | >120 | >120 | >120 |
| A173 | >120 | >120 | >120 | >120 | >120 |
| A180 | >120 | >120 | >120 | 58 | 45 |
| A187 | >120 | >120 | >120 | >120 | 88 |
| A191 | >120 | >120 | >120 | >120 | >120 |
| A194 | 118 | >120 | >120 | 89 | >120 |
| A217 | >120 | >120 | 26 | 50 | 56 |
| A239 | 113 | >120 | >120 | >120 | 39 |
| A246 | 89 | >120 | 65 | 79 | 78 |
| A254 | >120 | >120 | >120 | >120 | >120 |
| A262 | >120 | >120 | 98 | >120 | 51 |
| A264 | >120 | >120 | >120 | >120 | 48 |
| A269 | >120 | >120 | >120 | 79 | >120 |
| A270 | >120 | >120 | >120 | >120 | >120 |
| A274 | >120 | >120 | >120 | >120 | >120 |
| X | >120 | NA | NA | NA | NA |
| Enzalutamide | >120 | >120 | >120 | >120 | >120 |

The publications discussed herein are provided solely for their disclosure prior to the filing date of the present application. Nothing herein is to be construed as an admission that the present invention is not entitled to antedate such publication by virtue of prior invention.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A compound having the structure of formula (I-A): 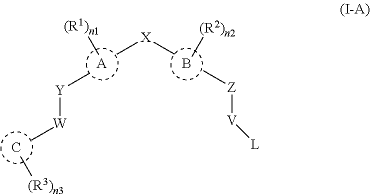
or a pharmaceutically acceptable salt, tautomer, stereoisomer or prodrug thereof, wherein:
A is a 4- to 15-membered ring selected from aryl, carbocyclyl, heteroaryl, or heterocyclyl;
B is a 4- to 15-membered ring selected from aryl, carbocyclyl, heteroaryl, or heterocyclyl;
C is a 3- to 10-membered aryl, carbocyclyl, heteroaryl, or heterocyclyl;
X is a bond, —(CR⁵R⁶)ₜ—, —C(═O)—, or —NR⁷;
Y is a bond, —(CR⁸R⁹)ₘ—, —O—, —S—, —S(═O)—, —SO₂—, —NR⁷—, or —N(COCH₃)—;
W is a bond, —(CR^{8a}R^{9a})ₘ—, —C(═O)—, —NR⁷—, —N(R⁷)CO—, —CONR⁷—, or —NSO₂R⁷—;
Z is a bond, —CH₂—, —C(CH₃)H—, —O—, —S—, —NH—, —NCH₃—, or —N(COCH₃)—;
V is a bond, —(CR^{8a}R^{9a})ₘ—, —CR^{8a}═CR^{9a}—, or phenylene;
L is hydrogen, halogen, —CF₂R¹⁰, —CF₃, —CN, —OR¹⁰, —SO₂R¹⁷, —NR¹¹R¹², or —CONR¹¹R¹²;
R¹ and R² are each independently hydrogen, halogen, —CN, —CF₃, —OH, optionally substituted C₁-C₆ alkyl, optionally substituted C₁-C₆ alkoxy, optionally substituted —(C₁-C₆ alkyl)-(C₁-C₆ alkoxy), optionally substituted —(C₁-C₆ alkyl)-OH, oxo, —NR¹³R¹, optionally substituted —(C₁-C₆ alkyl)-NR¹³R¹⁴, —NR¹⁴SO₂R¹⁶, optionally substituted —(C₁-C₆ alkyl)NR¹⁴SO₂R¹⁶, —NR¹⁴COR¹⁶, optionally substituted —(C₁-C₆ alkyl)-NR¹⁴COR¹⁶, —CONR¹³R¹⁴, optionally substituted —(C₁-C₆ alkyl)-CONR¹⁴R¹⁵, SO₂NR¹⁴R¹⁵, optionally substituted —(C₁-C₆ alkyl)-SO₂NR¹⁴R¹⁵, optionally substituted —SO₂R¹⁶ or optionally substituted —(C₁-C₆ alkyl)-SO₂R¹⁶;
R³ is halogen, oxo, ═S, ═NR¹⁶, —CN, —CF₃, —OH, —S(C₁-C₃ alkyl), C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴SO₂R¹⁶, —(C₁-C₃ alkyl)NR¹⁴SO₂R¹⁶, —NR¹⁴COR¹⁶, —NR¹⁴COOR¹⁶, —(C₁-C₆ alkyl)-NR¹⁴COR¹⁶, —CONR¹⁴R⁵, —(C₁-C₃ alkyl)-CONR¹⁴R⁵, —SO₂NR¹⁴R¹⁵, —(C₁-C₃ alkyl)-SO₂NR¹⁴R¹⁵, —SO₂(C₁-C₃ alkyl), —(C₁-C₆ alkyl)-SO₂(C₁-C₃ alkyl), optionally substituted carbocyclyl, optionally substituted heterocyclyl, optionally substituted aryl, or optionally substituted heteroaryl;
R⁵ and R⁶ are each independently hydrogen, halogen, —OH, optionally substituted C₁-C₃ alkyl, optionally substituted C₂-C₃ alkenyl, optionally substituted C₂-C₃ alkynyl, or C₁-C₃ alkoxy; or R⁵ and R⁶ taken together form an optionally substituted 3- to 6-membered carbocyclyl or heterocyclyl;
R⁷ is hydrogen, C₁-C₆ alkyl, C₂-C₆ alkenyl, or C₂-C₆ alkynyl;
R⁸ and R⁹ are each independently hydrogen, halogen, or C₁-C₃ alkyl;
R^{8a} and R^{9a} are each independently hydrogen, —OH, halogen, C₁-C₃ alkyl, C₂-C₃ alkenyl, C₂-C₃ alkynyl, C₁-C₃ alkoxy, —NR¹³R¹⁴, —(C₁-C₃ alkyl)-NR¹³R¹⁴, —NR¹⁴COR¹⁶, —(C₁-C₃ alkyl)-NR¹⁴COR¹⁶, —CONR¹⁴R¹⁵, or —(C₁-C₃ alkyl)-CONR¹⁴R¹⁵; or R^{8a} and R^{9a} taken together form an optionally substituted 3- to 6-membered carbocyclyl or heterocyclyl;
R¹⁰ is hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl;
R¹¹ and R¹² are each independently hydrogen, optionally substituted C₁-C₆ alkyl, optionally substituted C₂-C₆ alkenyl, optionally substituted C₂-C₆ alkynyl, optionally substituted carbocyclyl, optionally substituted heterocyclyl, optionally substituted aryl, or optionally substituted heteroaryl; or R¹¹ and R¹² taken together form an optionally substituted heterocyclyl which optionally contains one or two additional heteroatoms selected from N, S, or O;
R¹³, R¹⁴ and R¹⁵ are each independently hydrogen, C₁-C₃ alkyl, C₂-C₃ alkenyl, or C₂-C₃ alkynyl; or R¹⁴ and R¹⁵ taken together form an optionally substituted 3- to 6-membered heterocyclyl;
R¹⁶ is hydrogen, optionally substituted C₁-C₃ alkyl, optionally substituted C₂-C₃ alkenyl, optionally substituted C₂-C₃ alkynyl, C₃-C₆ cycloalky, or phenyl;
R¹⁷ is hydrogen, optionally substituted C₁-C₃ alkyl, optionally substituted C₂-C₃ alkenyl, or optionally substituted C₂-C₃ alkynyl;
each m is independently 0, 1, or 2;
n1 and n2 are each independently 0, 1, or 2;
n3 is 0, 1, 2, 3, 4 or 5; and
t is 0, 1 or 2.

2.-4. (canceled)

5. The compound of claim 1 having the structure of formula (A-II): 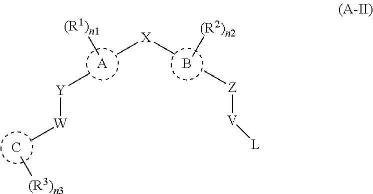
or a pharmaceutically acceptable salt, tautomer, stereoisomer or prodrug thereof, wherein:
A is a fused or a bridged bicyclic 5- to 15-membered ring selected from aryl, carbocyclyl, heteroaryl, or heterocyclyl;
B is a phenyl or a 6-membered heteroaryl or heterocyclyl;
C is a 3- to 10-membered aryl, carbocyclyl, heteroaryl, or heterocyclyl;
X is a bond, —CH₂—, —C(CH₃)₂—, or —C(═O)—;
Y is a bond, —CH₂—, —NH—, or —O—;
W is a bond, —CH₂—, —CH₂CH₂—, —C(CH₃)H—, —N(R⁷)CO—, or —CONR⁷—;
Z is a bond or —O—;
V is a bond, —CH₂(CR^{8a′}R^{9a′})—, or —(CR^{8a}R^{9a})ₘ—;
L is hydrogen, halogen, —CF₂R¹⁰, —CF₃, —CN, —OR¹⁰, —NR¹¹R¹², or —CONR¹¹R¹²;
R¹ and R² are each independently hydrogen, halogen, oxo, —CN, or —CF₃;
at least one R³ is —CH₂NHSO₂CH₃, —CH₂N(CH₃)SO₂CH₃, —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁- C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), or —N(CH₃)COO(C₁-C₃ alkyl);
R⁷ is hydrogen or C₁-C₃ alkyl;
R^{8a} and R^{9a} are each independently hydrogen, halogen, or C₁-C₃ alkyl;
R^{8a′} and R^{9a′} taken together form an optionally substituted 3- to 6-membered carbocyclyl or heterocyclyl containing one, two, or three heteroatoms selected from N, S, or O;
R¹⁰ is hydrogen, or C₁-C₃ alkyl;
R¹¹ and R¹² are each independently hydrogen or C₁-C₃ alkyl; or R¹¹ and R¹² taken together form an optionally substituted heterocyclyl which optionally contains one or two additional heteroatoms selected from N, S, or O;
m is 0, 1, or 2;
n1 and n2 are each independently 0, 1, or 2; and
n3 is 1, 2, 3, 4 or 5.

6. The compound of claim 1 having the structure of formula (A-III): 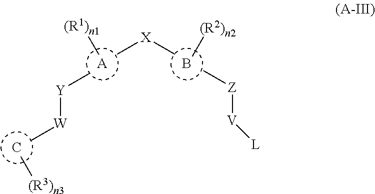
or a pharmaceutically acceptable salt, tautomer, stereoisomer or prodrug thereof, wherein:
A is a fused or a bridged bicyclic 5- to 15-membered ring selected from aryl, carbocyclyl, heteroaryl, or heterocyclyl;
B is a phenyl or a 6-membered heteroaryl or heterocyclyl;
C is a 4- to 10-membered aryl, carbocyclyl, heteroaryl, or heterocyclyl;
X is a bond, —CH₂—, —C(CH₃)₂—, or —C(═O)—;
Y is a bond, —CH₂—, —C(CH₃)H—, —NH—, or —O—;
W is —CH₂—, —CH₂CH₂—, —C(CH₃)H—, —NR⁷—, —N(R⁷)CO—, or —CONR⁷—;
Z is a bond or —O—;
V is a bond, —CH₂(CR^{8a′}R^{9a′})—, —CR^{8a}═CR^{9a}—, or —(CR^{8a}R^{9a})ₘ—;
L is hydrogen, halogen, —CF₂R¹⁰, —CF₃, —CN, —OR¹⁰, —NR¹¹R¹², —SO₂R¹⁷, or —CONR¹¹R¹²;
R¹ and R² are each independently hydrogen, halogen, oxo, —CN, or —CF₃;
at least one R³ is —CH₂NHSO₂CH₃, —CH₂N(CH₃)SO₂CH₃, —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁- C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), or —N(CH₃)COO(C₁-C₃ alkyl);
R⁷ is hydrogen or C₁-C₃ alkyl;
R^{8a} and R^{9a} are each independently hydrogen, halogen, or C₁-C₃ alkyl;
R^{8a′} and R^{9a′} taken together form an optionally substituted 3- to 6-membered carbocyclyl or heterocyclyl containing one, two, or three heteroatoms selected from N, S, or O;
R¹⁰ is hydrogen, or C₁-C₃ alkyl;
R¹¹ and R¹² are each independently hydrogen or C₁-C₃ alkyl; or R¹¹ and R¹² taken together form an optionally substituted heterocyclyl which optionally contains one or two additional heteroatoms selected from N, S, or O;
R¹⁷ is hydrogen or C₁-C₃ alkyl;
m is 0, 1, or 2;
n1 and n2 are each independently 0, 1, or 2; and
n3 is 1, 2, 3, 4 or 5.

7. The compound of claim 5, wherein:
X is bond or —C(CH₃)₂—;
Y and Z are each —O—;
V is —CH₂— or —CH₂CH₂—;
L is halogen;
R¹ and R² are each independently hydrogen, halogen, —CN, or —CF₃; and
R¹⁶ is hydrogen, C₁-C₃ alkyl, C₂-C₃ alkenyl, or C₂-C₃ alkynyl.

8. The compound of claim 7, wherein:
W is —CH₂— or —C(CH₃)H—;
V is —CH₂CH₂—; and
R¹ and R² are each independently hydrogen, halogen, —CN, or —CF₃.

9. The compound of claim 6, wherein:
Y is —CH₂— or —C(CH₃)H—;
W is —NH—;
X is bond or —C(CH₃)₂—;
Z is —O—;
V is —CH₂— or —CH₂CH₂—;
L is halogen; and
R¹ and R² are each independently hydrogen, halogen, —CN, or —CF₃.

10. (canceled)

11. The compound of claim 5, wherein A is a 6,6-fused ring, 6,5-fused ring, or 5,6-fused ring, each optionally substituted with one or two R¹.

12. The compound of claim 5, wherein A is 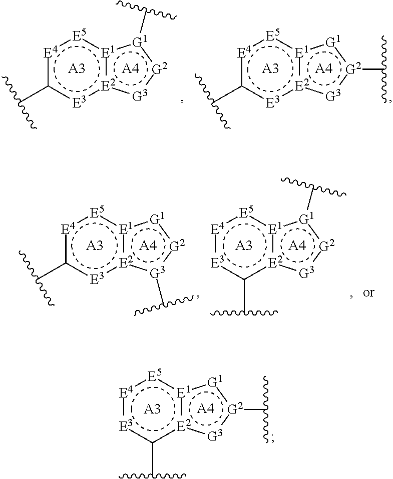
wherein:
ring A3 is aromatic;
E¹, E², E³, E⁴, and E⁵ are each independently, C, CR¹ or N;
G¹, G², and G³, are each independently, C, CR¹, C(R¹)₂, O, S, N, or NR¹;
wherein at least two of E¹, E², E³, E⁴, and E⁵ is C or CR¹; and
wherein at least two of E¹, E², G¹, G², and G³, is C, CR¹ or C(R¹)₂.

13. The compound of claim 5, wherein A is 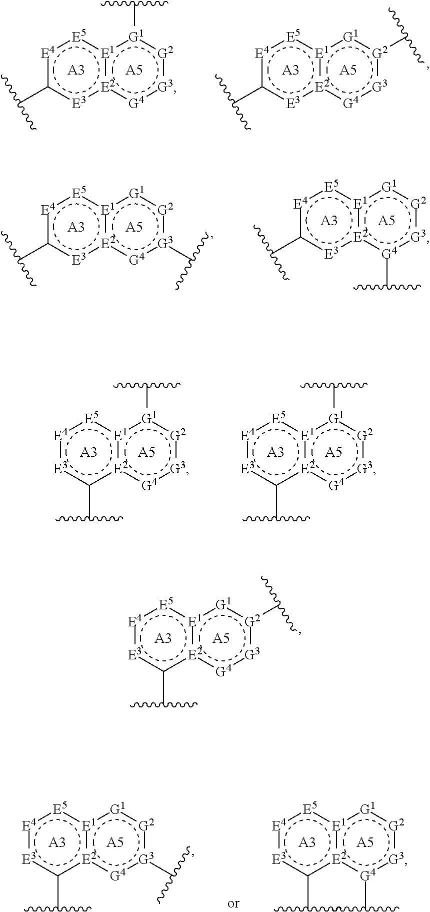
wherein:
ring A3 is aromatic;
E¹, E², E³, E⁴, and E⁵ are each independently, C, CR¹ or N;
G¹, G², G³, and G⁴, are each independently, C, CR¹, C(R¹)₂, O, S, N, or NR¹;
wherein at least two of E¹, E², E³, E⁴, and E⁵ is C or CR¹; and
wherein at least three of E¹, E², G¹, G², G³, and G⁴, is C, CR¹ or C(R¹)₂.

14. The compound of claim 5, wherein A is a ring selected from bicyclo[1.1.1]pentane, 4,5,6,7-tetrahydroindole, indoline, indole, pyrazolo[1,5-a]pyridine, imidazo[1,2-a]pyridine, indazole, benzo[d]imidazole, benzo[d]isoxazole, benzo[b]thiophene, 1,3-dihydroisobenzofuran, quinazoline, 3,4-dihydrobenzo[b][1,4]oxazine, benzo[d][1,2,3]triazole, naphthalene, 1,2-dihydronaphthalene, 1,2,3,4-tetrahydronaphthalene, 2,3-dihydroindene, 2,3-dihydrobenzo[b][1,4]dioxine, isoindoline, or isoindolin-1-one, each ring is optionally substituted with one or two R¹.

15. The compound of claim 5, wherein A is 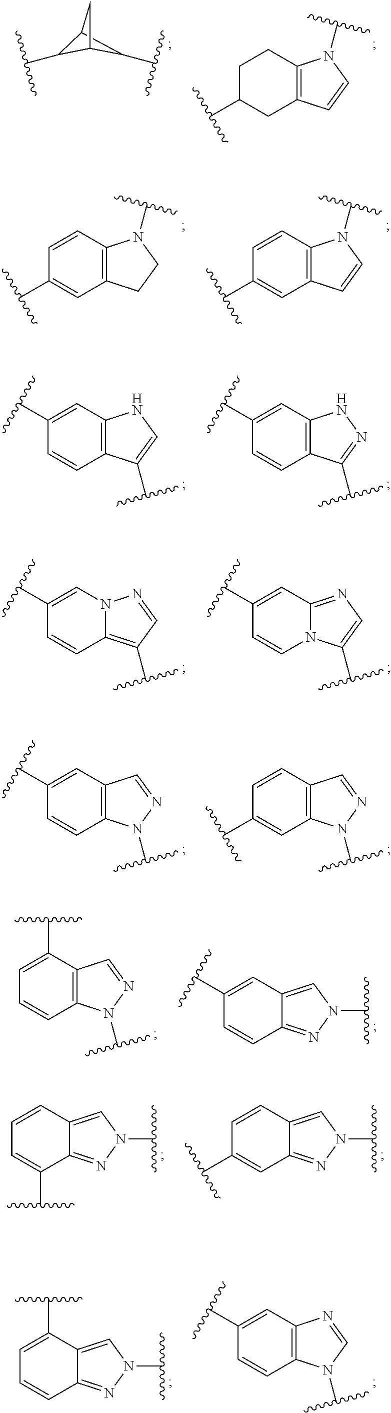 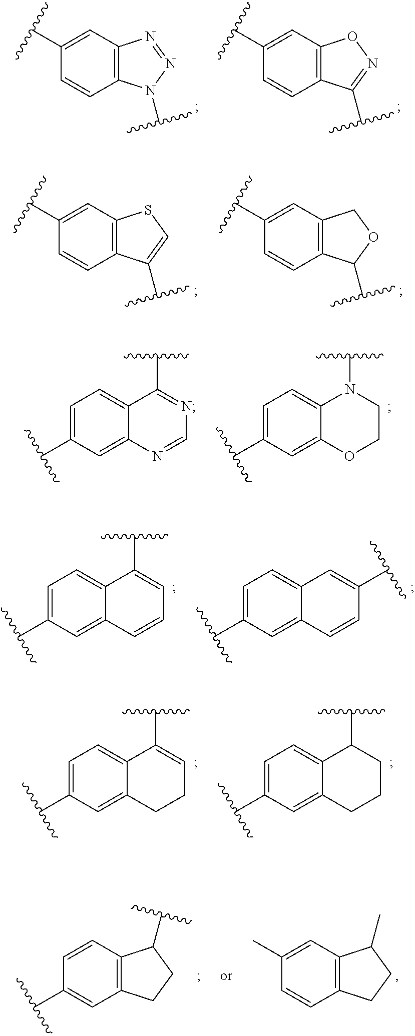
each ring is optionally substituted with one or two R¹.

16. The compound of claim 5, wherein C is

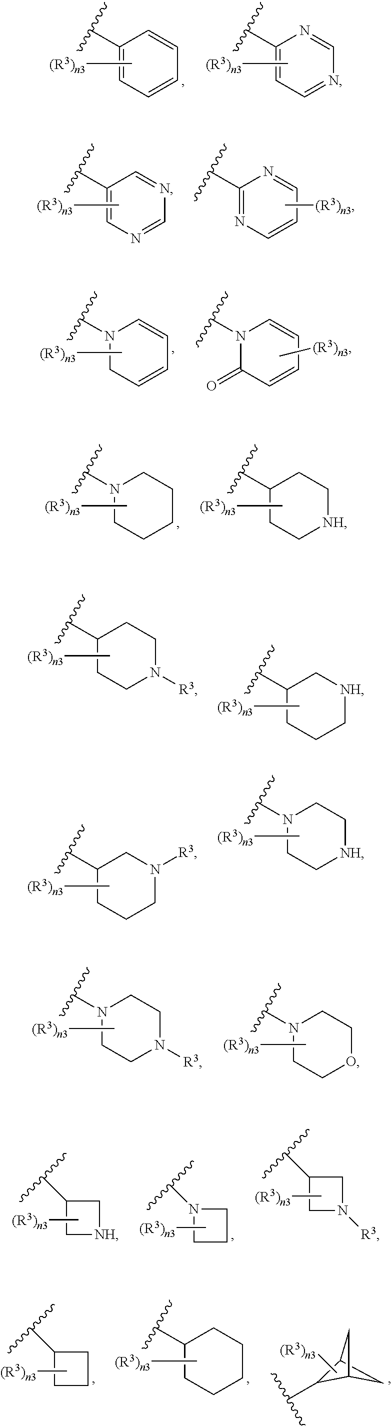

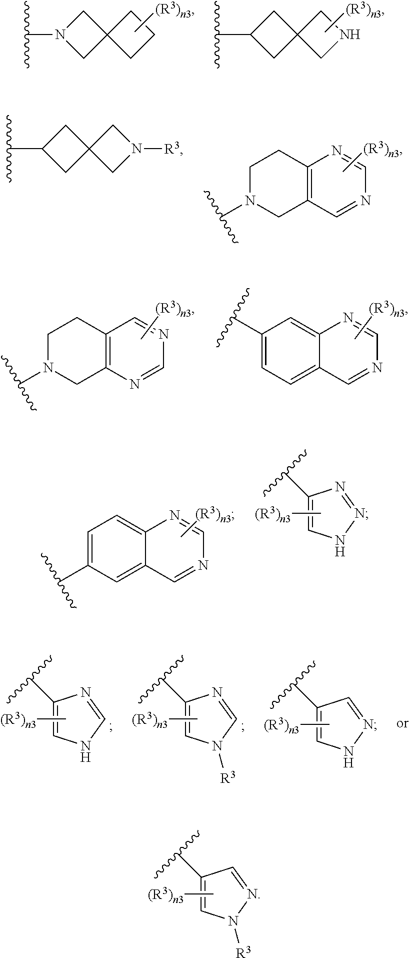

17. The compound of claim 1 having the structure of formula (B-I): 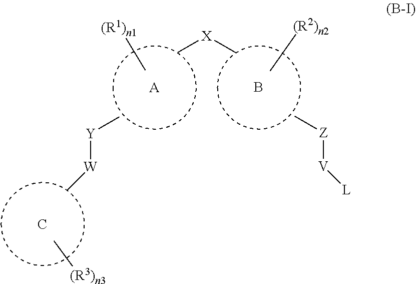
or a pharmaceutically acceptable salt, tautomer, stereoisomer or prodrug thereof, wherein:
A is a phenyl or a 6-membered heteroaryl;
B is a fused or a bridged bicyclic 5- to 15-membered ring selected from aryl, carbocyclyl, heteroaryl, or heterocyclyl;
C is a heteroaryl ring;
X is a bond, —CH₂—, —C(CH₃)₂—, or —C(═O)—;
Y is a bond or —O—;
W is —CH₂— or —C(CH₃)H—;
Z is a bond or —O—;
V is a bond or —CH₂— and L is halogen, —CHCl₂, —CCl₃, or —CF₃; or
V is —CH₂CH₂— and L is halogen;
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —NHCOO(C₁-C₃ alkyl), —N(CH₃)CO(C₁-C₃ alkyl), or —N(CH₃)COO(C₁-C₃ alkyl);
n1 and n2 are each independently 0, 1, or 2; and
n3 is 1, 2, 3, 4 or 5.

18.-19. (canceled)

20. The compound of claim 17, wherein B is a 6,6-fused ring, 6,5-fused ring, or 5,6-fused ring, each optionally substituted with one or more R².

21.-23. (canceled)

24. The compound of claim 17, wherein B is: 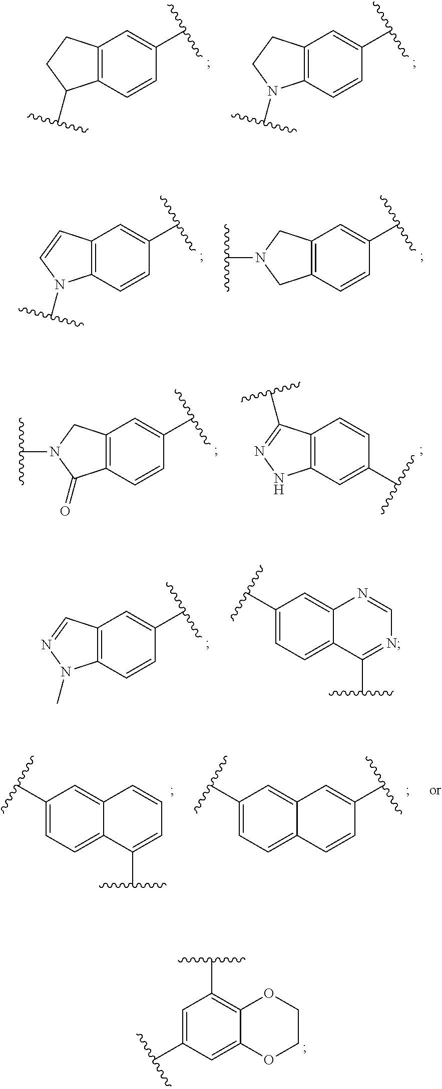
wherein each ring is optionally substituted with one or two R².

25. (canceled)

26. The compound of claim 5, wherein C is a pyrimidine ring.

27. The compound of claim 5, wherein R¹ and R² are each independently Cl, —CN, or —CF₃.

28. (canceled)

29. The compound of claim 5, wherein at least one R³ is —CH₂NHSO₂CH₃, —CH₂N(CH₃)SO₂CH₃, —NHSO₂CH₃, —NHSO₂CH₂CH₃, —SO₂NH₂, or —SO₂CH₃.

30.-36. (canceled)

37. The compound of claim 1 having the structure of: 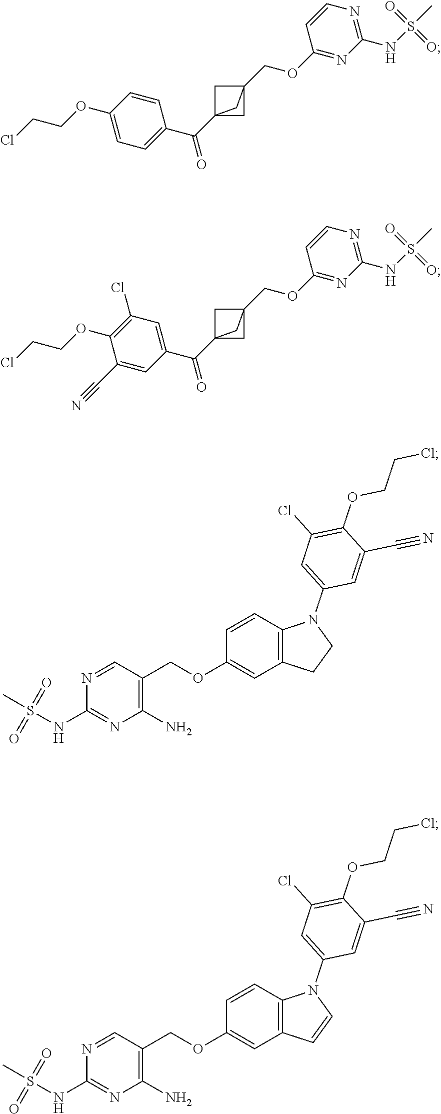 [SMILES: CS(=O)(=O)Nc1nccc(OCC23CC(C(=O)c4ccc(OCCCl)cc4)(C2)C3)n1 ; CS(=O)(=O)Nc1nccc(OCC23CC(C(=O)c4cc(Cl)c(OCCCl)c(C#N)c4)(C2)C3)n1 ; CS(=O)(=O)Nc1ncc(COc2ccc3c(c2)CCN3c2cc(Cl)c(OCCCl)c(C#N)c2)c(N)n1 ; CS(=O)(=O)Nc1ncc(COc2ccc3c(ccn3-c3cc(Cl)c(OCCCl)c(C#N)c3)c2)c(N)n1] 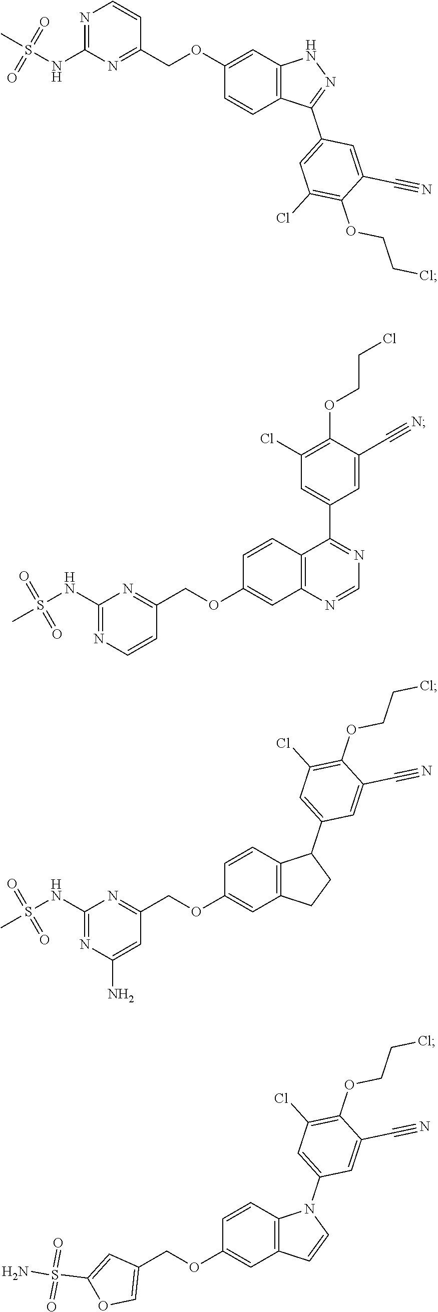 [SMILES: CS(=O)(=O)Nc1nccc(COc2ccc3c(-c4cc(Cl)c(OCCCl)c(C#N)c4)n[nH]c3c2)n1 ; CS(=O)(=O)Nc1nccc(COc2ccc3c(-c4cc(Cl)c(OCCCl)c(C#N)c4)ncnc3c2)n1 ; CS(=O)(=O)Nc1nc(N)cc(COc2ccc3c(c2)CCC3c2cc(Cl)c(OCCCl)c(C#N)c2)n1 ; N#Cc1cc(-n2ccc3cc(OCc4coc(S(N)(=O)=O)c4)ccc32)cc(Cl)c1OCCCl] 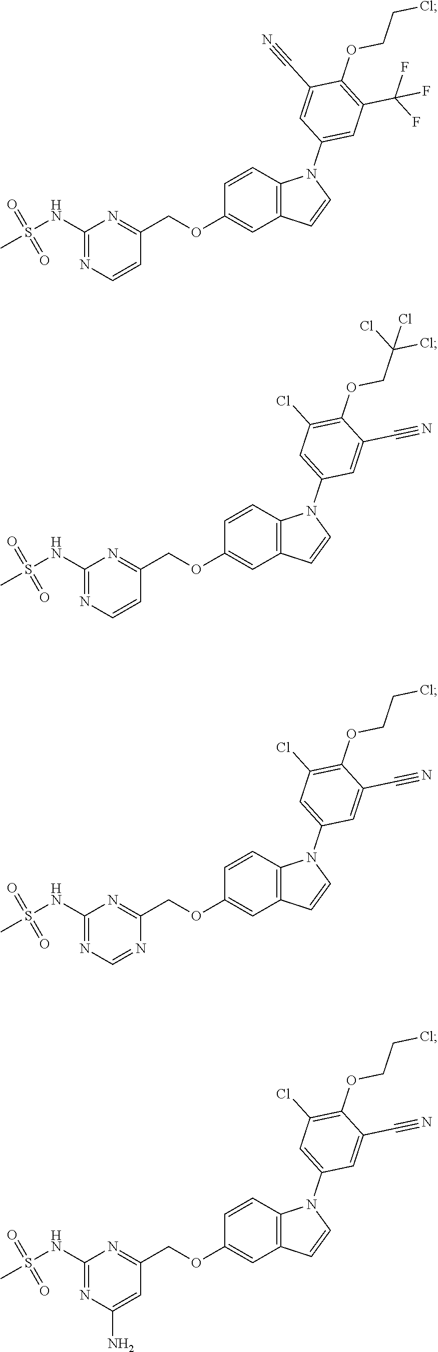 [SMILES: CS(=O)(=O)Nc1nccc(COc2ccc3c(ccn3-c3cc(C#N)c(OCCCl)c(C(F)(F)F)c3)c2)n1 ; CS(=O)(=O)Nc1nccc(COc2ccc3c(ccn3-c3cc(Cl)c(OCC(Cl)(Cl)Cl)c(C#N)c3)c2)n1 ; CS(=O)(=O)Nc1ncnc(COc2ccc3c(ccn3-c3cc(Cl)c(OCCCl)c(C#N)c3)c2)n1 ; CS(=O)(=O)Nc1nc(N)cc(COc2ccc3c(ccn3-c3cc(Cl)c(OCCCl)c(C#N)c3)c2)n1] 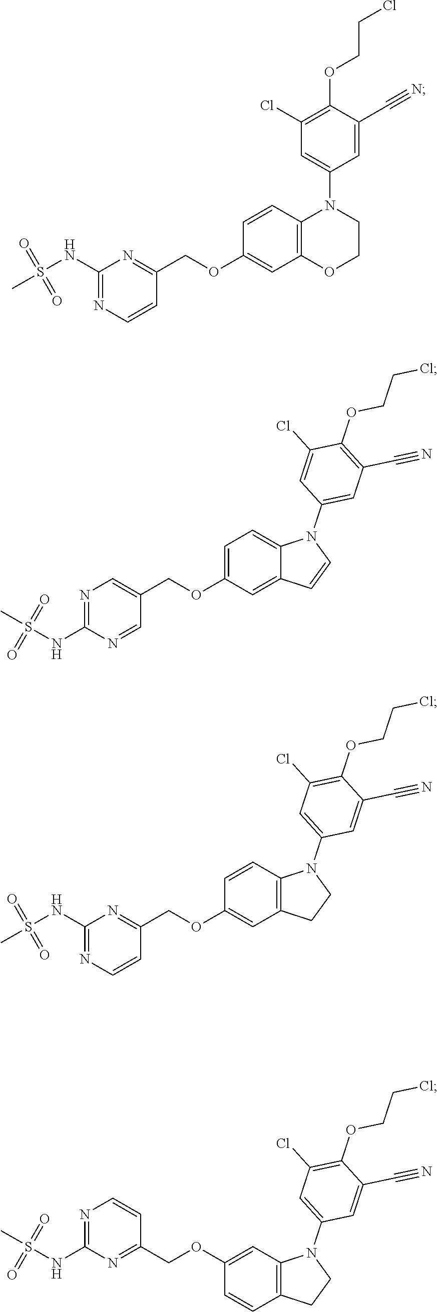 [SMILES: CS(=O)(=O)Nc1nccc(COc2ccc3c(c2)OCCN3c2cc(Cl)c(OCCCl)c(C#N)c2)n1 ; CS(=O)(=O)Nc1ncc(COc2ccc3c(ccn3-c3cc(Cl)c(OCCCl)c(C#N)c3)c2)cn1 ; CS(=O)(=O)Nc1nccc(COc2ccc3c(c2)CCN3c2cc(Cl)c(OCCCl)c(C#N)c2)n1 ; CS(=O)(=O)Nc1nccc(COc2ccc3c(c2)N(c2cc(Cl)c(OCCCl)c(C#N)c2)CC3)n1] 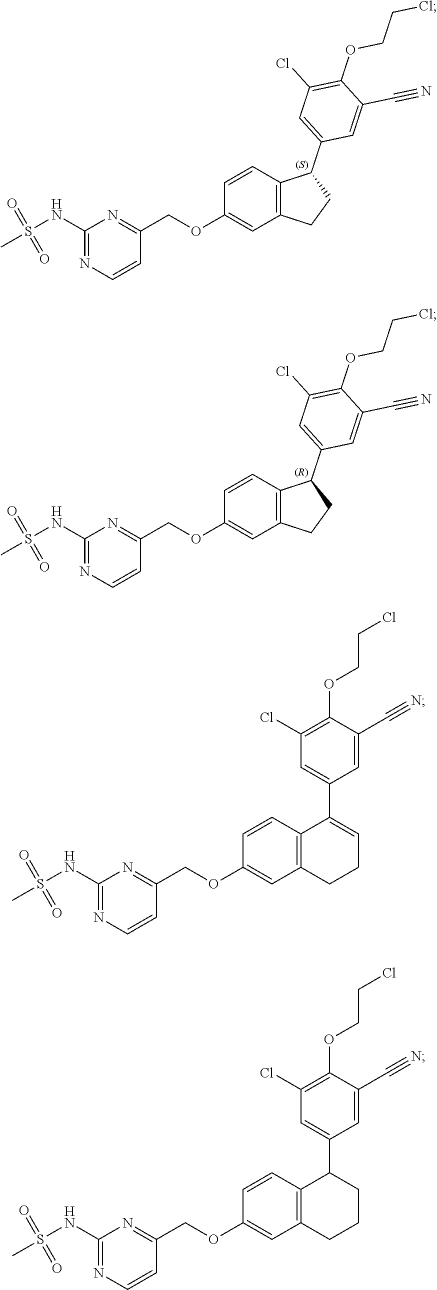 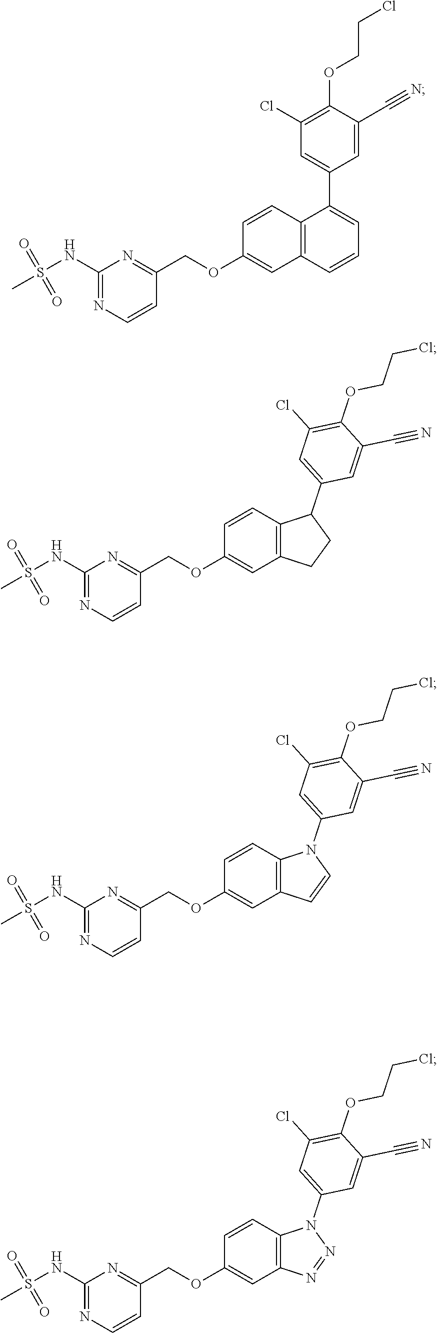 [SMILES: CS(=O)(=O)Nc1nccc(COc2ccc3c(-c4cc(Cl)c(OCCCl)c(C#N)c4)cccc3c2)n1 ; CS(=O)(=O)Nc1nccc(COc2ccc3c(c2)CCC3c2cc(Cl)c(OCCCl)c(C#N)c2)n1 ; CS(=O)(=O)Nc1nccc(COc2ccc3c(ccn3-c3cc(Cl)c(OCCCl)c(C#N)c3)c2)n1 ; CS(=O)(=O)Nc1nccc(COc2ccc3c(c2)nnn3-c2cc(Cl)c(OCCCl)c(C#N)c2)n1] 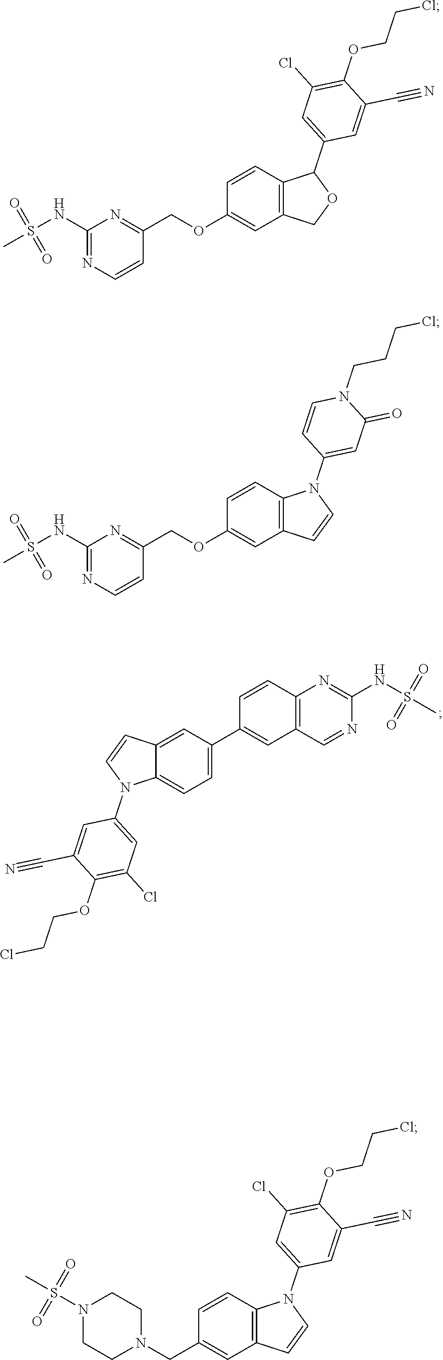 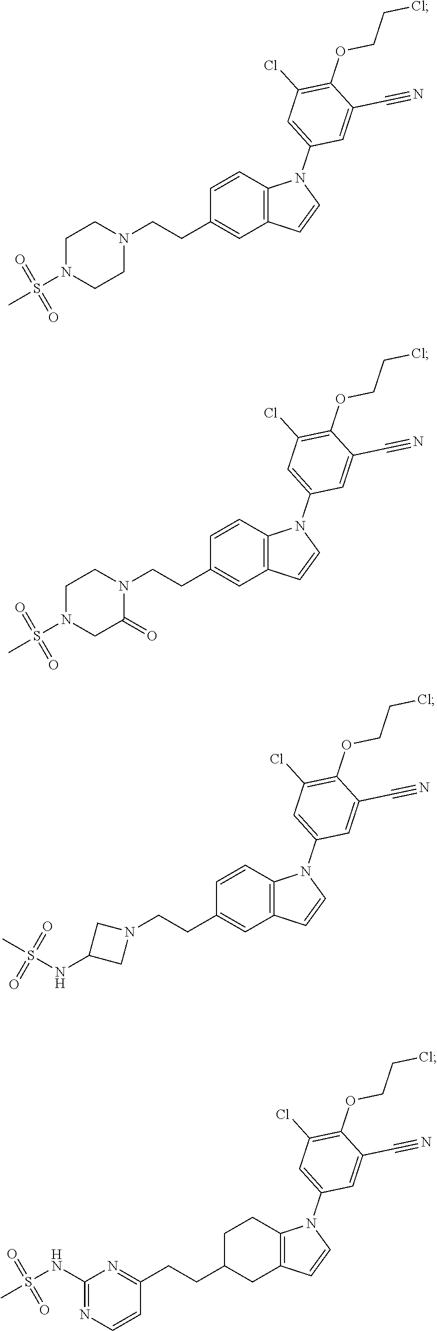 [SMILES: CS(=O)(=O)N1CCN(CCc2ccc3c(ccn3-c3cc(Cl)c(OCCCl)c(C#N)c3)c2)CC1 ; CS(=O)(=O)N1CCN(CCc2ccc3c(ccn3-c3cc(Cl)c(OCCCl)c(C#N)c3)c2)C(=O)C1 ; CS(=O)(=O)NC1CN(CCc2ccc3c(ccn3-c3cc(Cl)c(OCCCl)c(C#N)c3)c2)C1 ; CS(=O)(=O)Nc1nccc(CCC2CCc3c(ccn3-c3cc(Cl)c(OCCCl)c(C#N)c3)C2)n1] 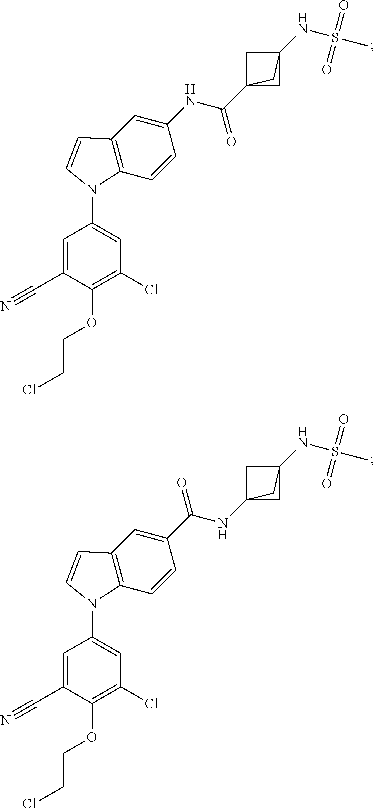 [SMILES: CS(=O)(=O)NC12CC(C(=O)Nc3ccc4c(ccn4-c4cc(Cl)c(OCCCl)c(C#N)c4)c3)(C1)C2 ; CS(=O)(=O)NC12CC(NC(=O)c3ccc4c(ccn4-c4cc(Cl)c(OCCCl)c(C#N)c4)c3)(C1)C2] 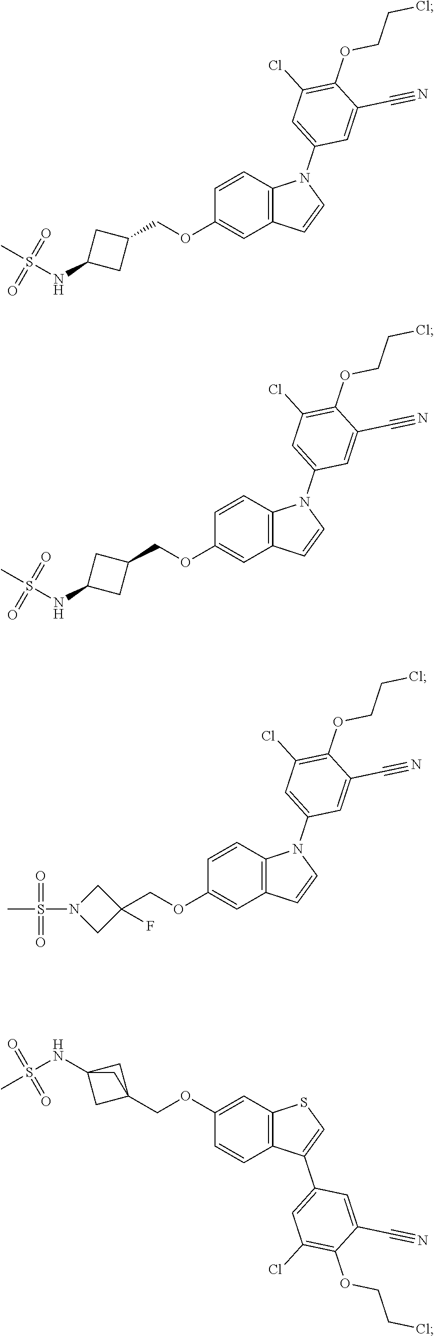 [SMILES: CS(=O)(=O)N[C@H]1C[C@H](COc2ccc3c(ccn3-c3cc(Cl)c(OCCCl)c(C#N)c3)c2)C1 ; CS(=O)(=O)N[C@H]1C[C@@H](COc2ccc3c(ccn3-c3cc(Cl)c(OCCCl)c(C#N)c3)c2)C1 ; CS(=O)(=O)N1CC(F)(COc2ccc3c(ccn3-c3cc(Cl)c(OCCCl)c(C#N)c3)c2)C1 ; CS(=O)(=O)NC12CC(COc3ccc4c(-c5cc(Cl)c(OCCCl)c(C#N)c5)csc4c3)(C1)C2] 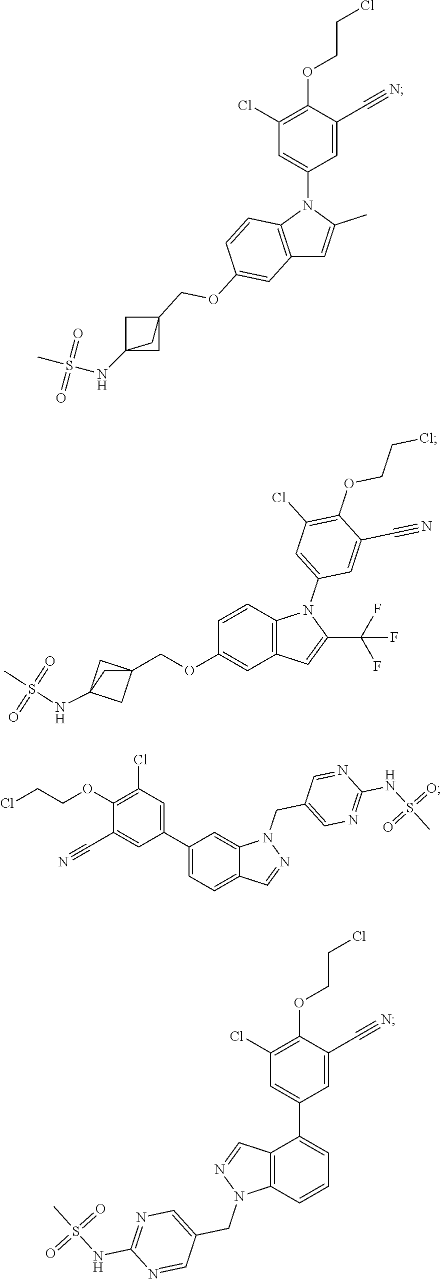 [SMILES: Cc1cc2cc(OCC34CC(NS(C)(=O)=O)(C3)C4)ccc2n1-c1cc(Cl)c(OCCCl)c(C#N)c1 ; CS(=O)(=O)NC12CC(COc3ccc4c(c3)cc(C(F)(F)F)n4-c3cc(Cl)c(OCCCl)c(C#N)c3)(C1)C2 ; CS(=O)(=O)Nc1ncc(Cn2ncc3ccc(-c4cc(Cl)c(OCCCl)c(C#N)c4)cc32)cn1 ; CS(=O)(=O)Nc1ncc(Cn2ncc3c(-c4cc(Cl)c(OCCCl)c(C#N)c4)cccc32)cn1] 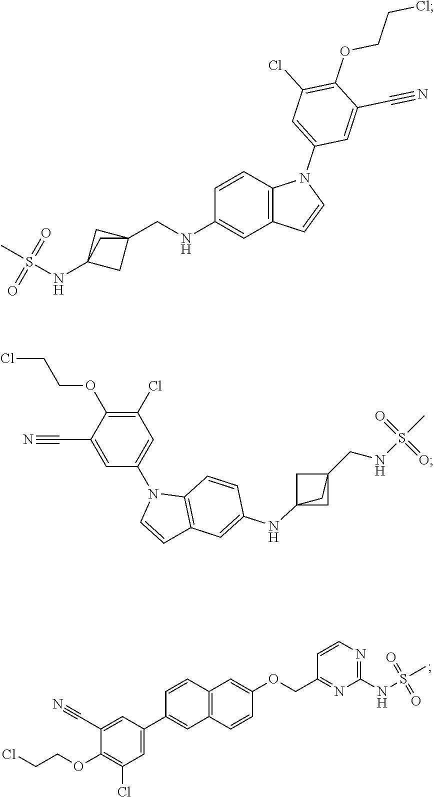 [SMILES: CS(=O)(=O)NC12CC(CNc3ccc4c(ccn4-c4cc(Cl)c(OCCCl)c(C#N)c4)c3)(C1)C2 ; CS(=O)(=O)NCC12CC(Nc3ccc4c(ccn4-c4cc(Cl)c(OCCCl)c(C#N)c4)c3)(C1)C2 ; CS(=O)(=O)Nc1nccc(COc2ccc3cc(-c4cc(Cl)c(OCCCl)c(C#N)c4)ccc3c2)n1] 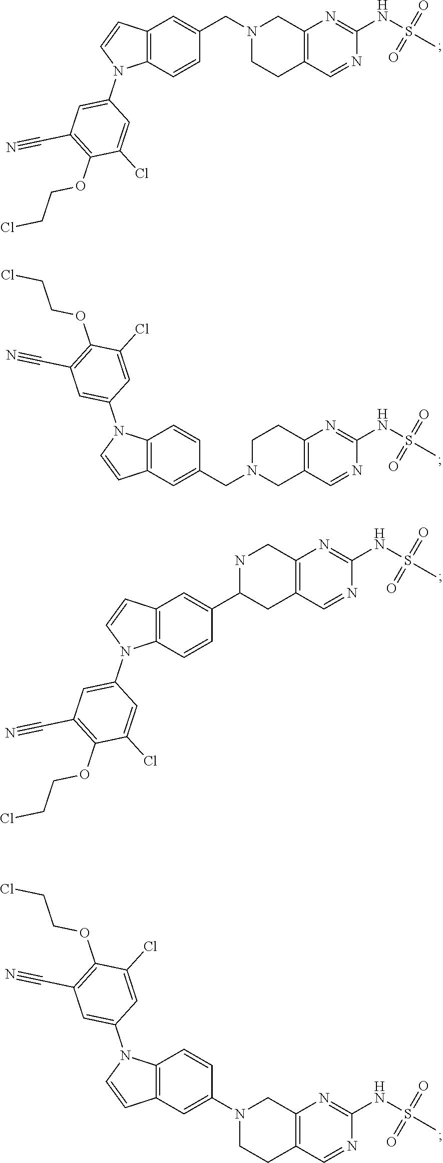 [SMILES: CS(=O)(=O)Nc1ncc2c(n1)CN(Cc1ccc3c(ccn3-c3cc(Cl)c(OCCCl)c(C#N)c3)c1)CC2 ; CS(=O)(=O)Nc1ncc2c(n1)CCN(Cc1ccc3c(ccn3-c3cc(Cl)c(OCCCl)c(C#N)c3)c1)C2 ; CS(=O)(=O)Nc1ncc2c(n1)CNC(c1ccc3c(ccn3-c3cc(Cl)c(OCCCl)c(C#N)c3)c1)C2 ; CS(=O)(=O)Nc1ncc2c(n1)CN(c1ccc3c(ccn3-c3cc(Cl)c(OCCCl)c(C#N)c3)c1)CC2] 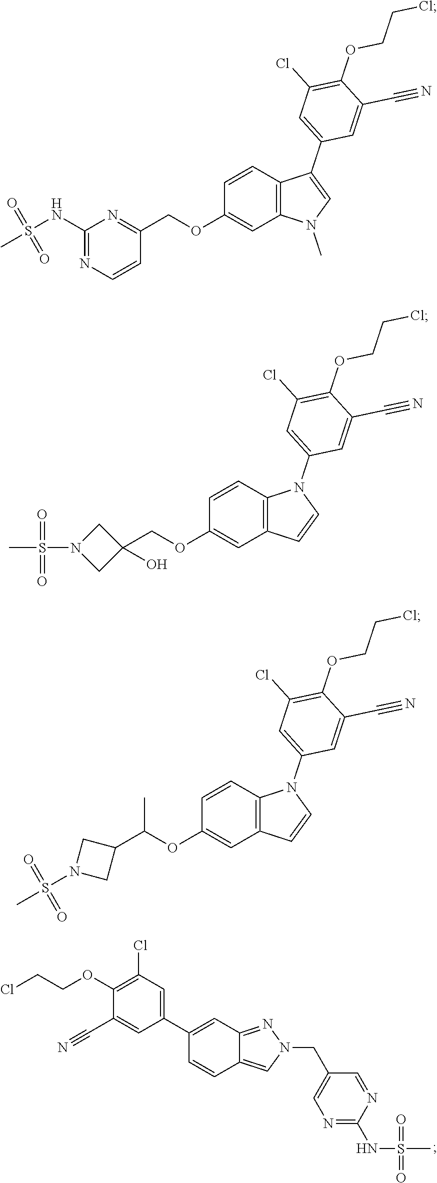 [SMILES: Cn1cc(-c2cc(Cl)c(OCCCl)c(C#N)c2)c2ccc(OCc3ccnc(NS(C)(=O)=O)n3)cc21 ; CS(=O)(=O)N1CC(O)(COc2ccc3c(ccn3-c3cc(Cl)c(OCCCl)c(C#N)c3)c2)C1 ; CC(Oc1ccc2c(ccn2-c2cc(Cl)c(OCCCl)c(C#N)c2)c1)C1CN(S(C)(=O)=O)C1 ; CS(=O)(=O)Nc1ncc(Cn2cc3ccc(-c4cc(Cl)c(OCCCl)c(C#N)c4)cc3n2)cn1] 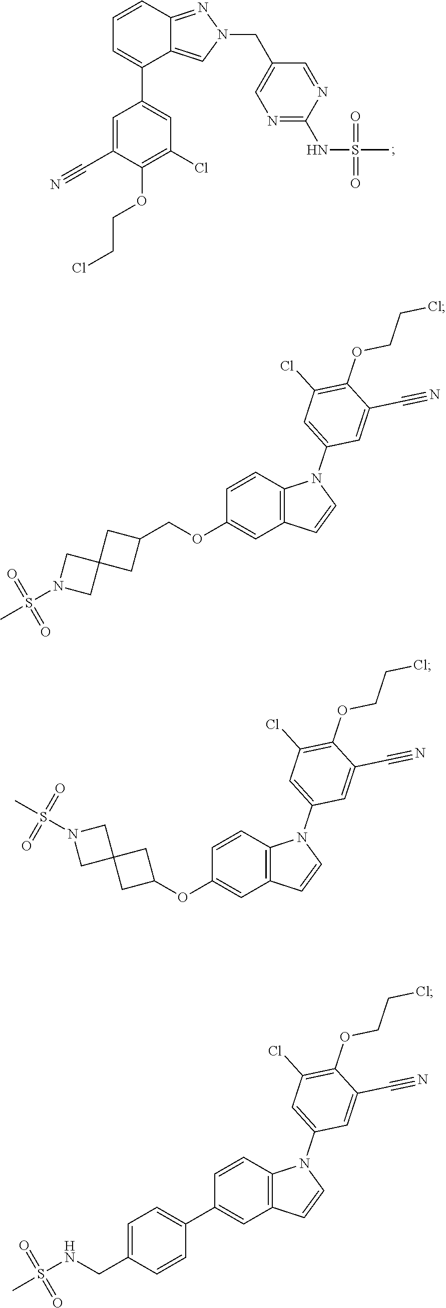 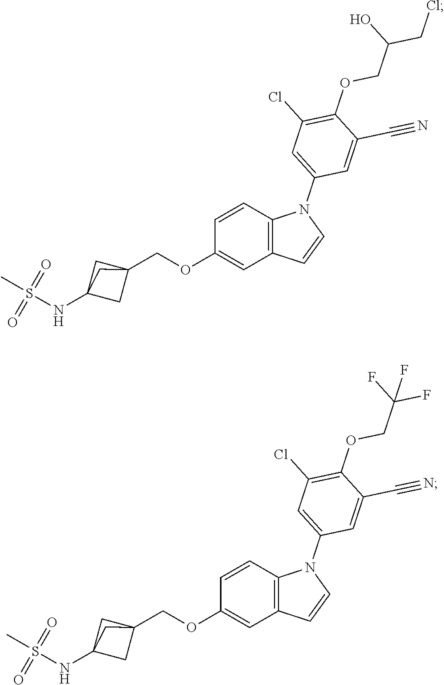 [SMILES: CS(=O)(=O)NC12CC(COc3ccc4c(ccn4-c4cc(Cl)c(OCC(O)CCl)c(C#N)c4)c3)(C1)C2 ; CS(=O)(=O)NC12CC(COc3ccc4c(ccn4-c4cc(Cl)c(OCC(F)(F)F)c(C#N)c4)c3)(C1)C2] 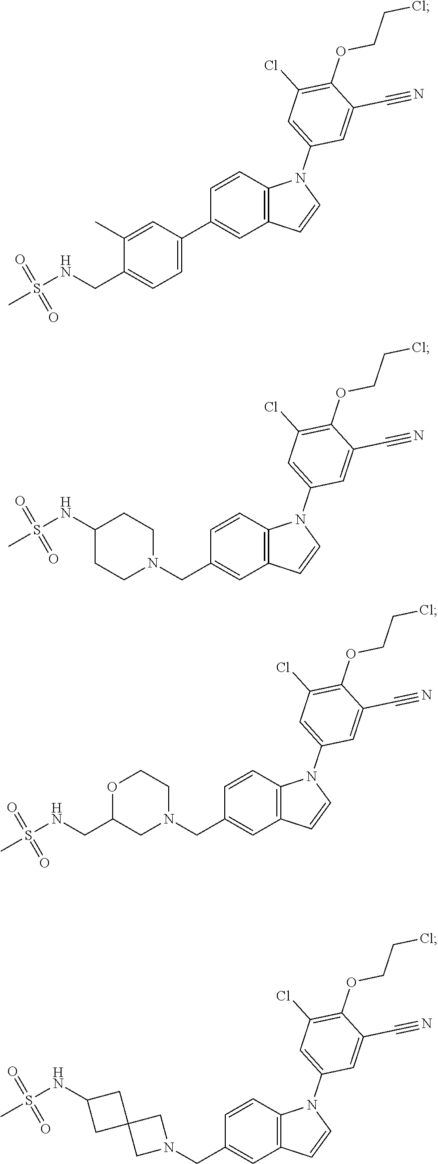 [SMILES: Cc1cc(-c2ccc3c(ccn3-c3cc(Cl)c(OCCCl)c(C#N)c3)c2)ccc1CNS(C)(=O)=O ; CS(=O)(=O)NC1CCN(Cc2ccc3c(ccn3-c3cc(Cl)c(OCCCl)c(C#N)c3)c2)CC1 ; CS(=O)(=O)NCC1CN(Cc2ccc3c(ccn3-c3cc(Cl)c(OCCCl)c(C#N)c3)c2)CCO1 ; CS(=O)(=O)NC1CC2(C1)CN(Cc1ccc3c(ccn3-c3cc(Cl)c(OCCCl)c(C#N)c3)c1)C2] 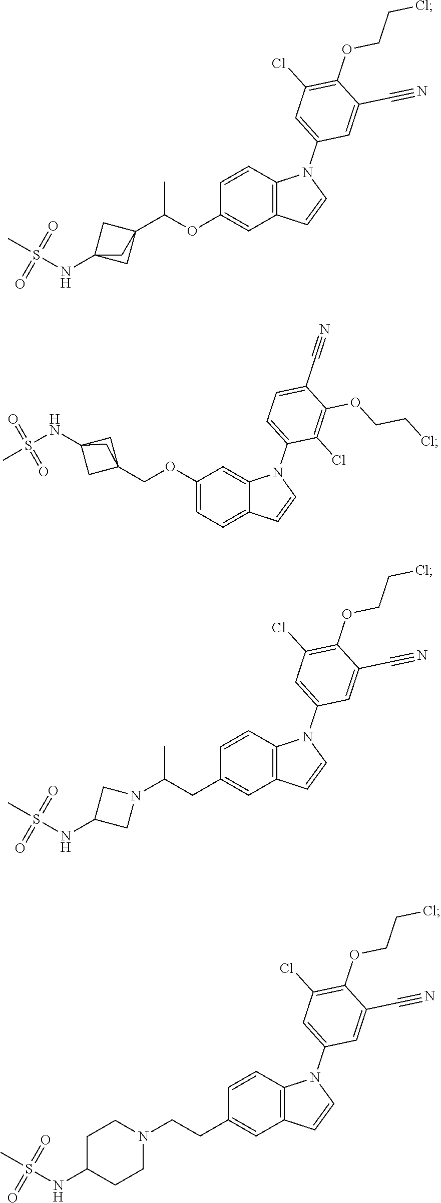 [SMILES: CC(Oc1ccc2c(ccn2-c2cc(Cl)c(OCCCl)c(C#N)c2)c1)C12CC(NS(C)(=O)=O)(C1)C2 ; CS(=O)(=O)NC12CC(COc3ccc4ccn(-c5ccc(C#N)c(OCCCl)c5Cl)c4c3)(C1)C2 ; CC(Cc1ccc2c(ccn2-c2cc(Cl)c(OCCCl)c(C#N)c2)c1)N1CC(NS(C)(=O)=O)C1 ; CS(=O)(=O)NC1CCN(CCc2ccc3c(ccn3-c3cc(Cl)c(OCCCl)c(C#N)c3)c2)CC1] 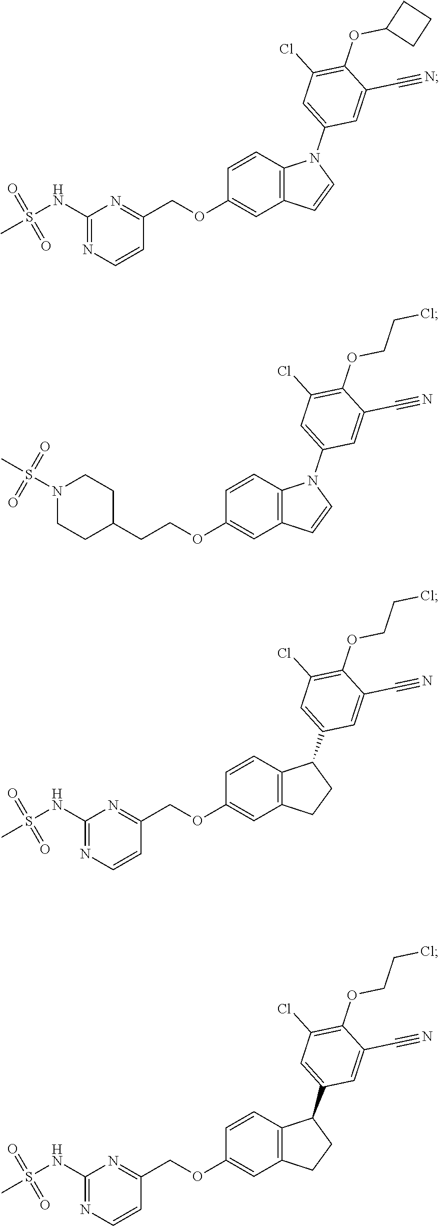 [SMILES: CS(=O)(=O)Nc1nccc(COc2ccc3c(ccn3-c3cc(Cl)c(OC4CCC4)c(C#N)c3)c2)n1 ; CS(=O)(=O)N1CCC(CCOc2ccc3c(ccn3-c3cc(Cl)c(OCCCl)c(C#N)c3)c2)CC1 ; CS(=O)(=O)Nc1nccc(COc2ccc3c(c2)CC[C@H]3c2cc(Cl)c(OCCCl)c(C#N)c2)n1 ; CS(=O)(=O)Nc1nccc(COc2ccc3c(c2)CC[C@@H]3c2cc(Cl)c(OCCCl)c(C#N)c2)n1] 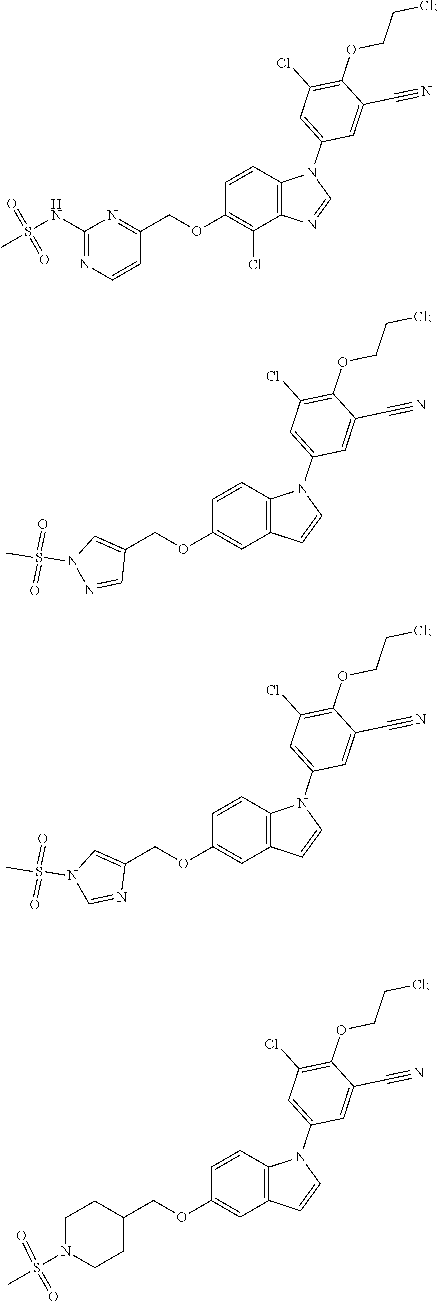 [SMILES: CS(=O)(=O)Nc1nccc(COc2ccc3c(ncn3-c3cc(Cl)c(OCCCl)c(C#N)c3)c2Cl)n1 ; CS(=O)(=O)n1cc(COc2ccc3c(ccn3-c3cc(Cl)c(OCCCl)c(C#N)c3)c2)cn1 ; CS(=O)(=O)n1cnc(COc2ccc3c(ccn3-c3cc(Cl)c(OCCCl)c(C#N)c3)c2)c1 ; CS(=O)(=O)N1CCC(COc2ccc3c(ccn3-c3cc(Cl)c(OCCCl)c(C#N)c3)c2)CC1] 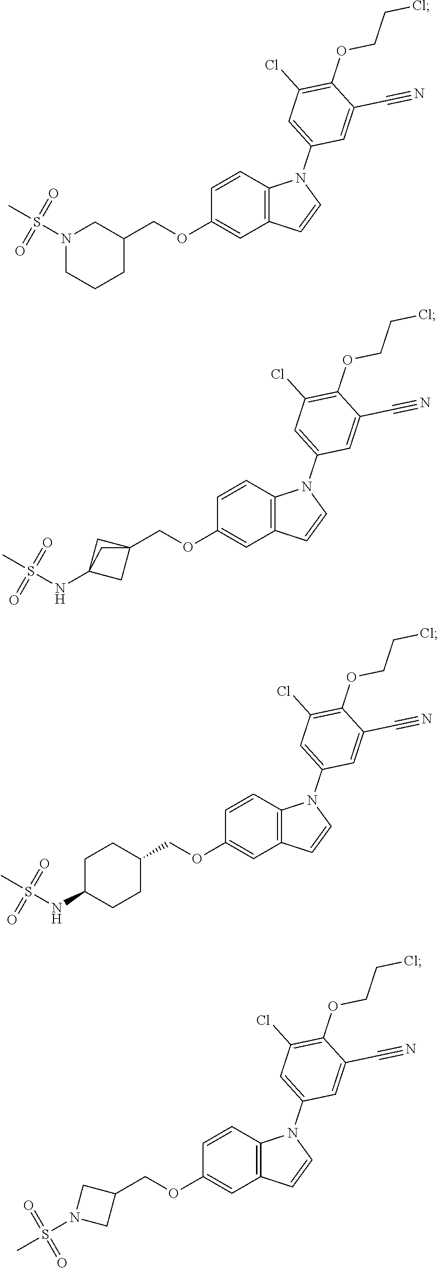 [SMILES: CS(=O)(=O)N1CCCC(COc2ccc3c(ccn3-c3cc(Cl)c(OCCCl)c(C#N)c3)c2)C1 ; CS(=O)(=O)NC12CC(COc3ccc4c(ccn4-c4cc(Cl)c(OCCCl)c(C#N)c4)c3)(C1)C2 ; CS(=O)(=O)N[C@H]1CC[C@H](COc2ccc3c(ccn3-c3cc(Cl)c(OCCCl)c(C#N)c3)c2)CC1 ; CS(=O)(=O)N1CC(COc2ccc3c(ccn3-c3cc(Cl)c(OCCCl)c(C#N)c3)c2)C1] 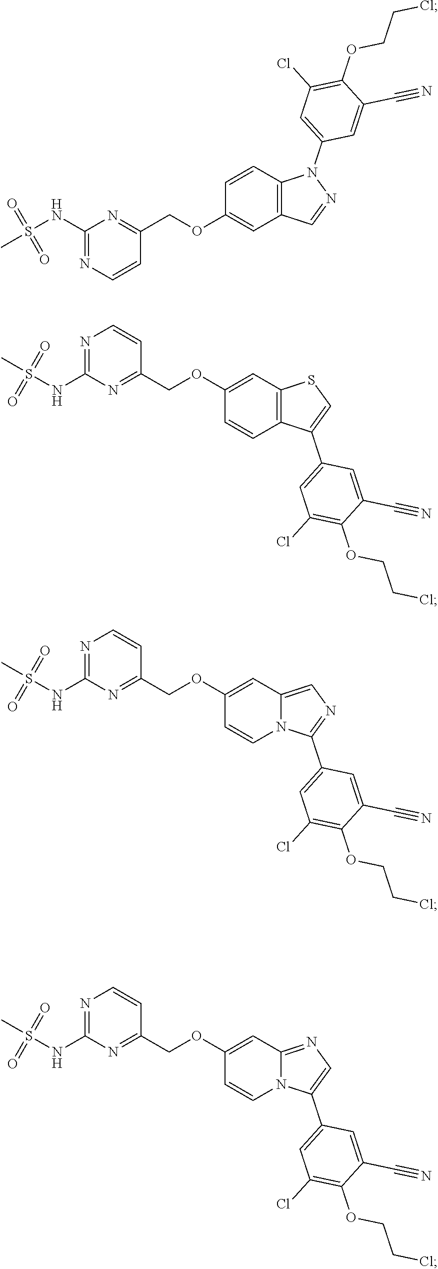 [SMILES: CS(=O)(=O)Nc1nccc(COc2ccc3c(cnn3-c3cc(Cl)c(OCCCl)c(C#N)c3)c2)n1 ; CS(=O)(=O)Nc1nccc(COc2ccc3c(-c4cc(Cl)c(OCCCl)c(C#N)c4)csc3c2)n1 ; CS(=O)(=O)Nc1nccc(COc2ccn3c(-c4cc(Cl)c(OCCCl)c(C#N)c4)ncc3c2)n1 ; CS(=O)(=O)Nc1nccc(COc2ccn3c(-c4cc(Cl)c(OCCCl)c(C#N)c4)cnc3c2)n1] 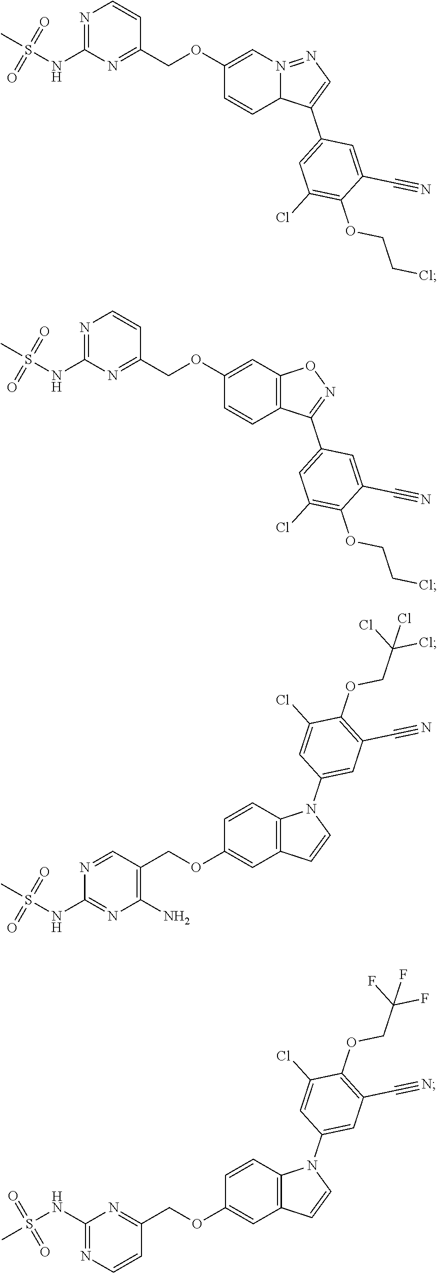 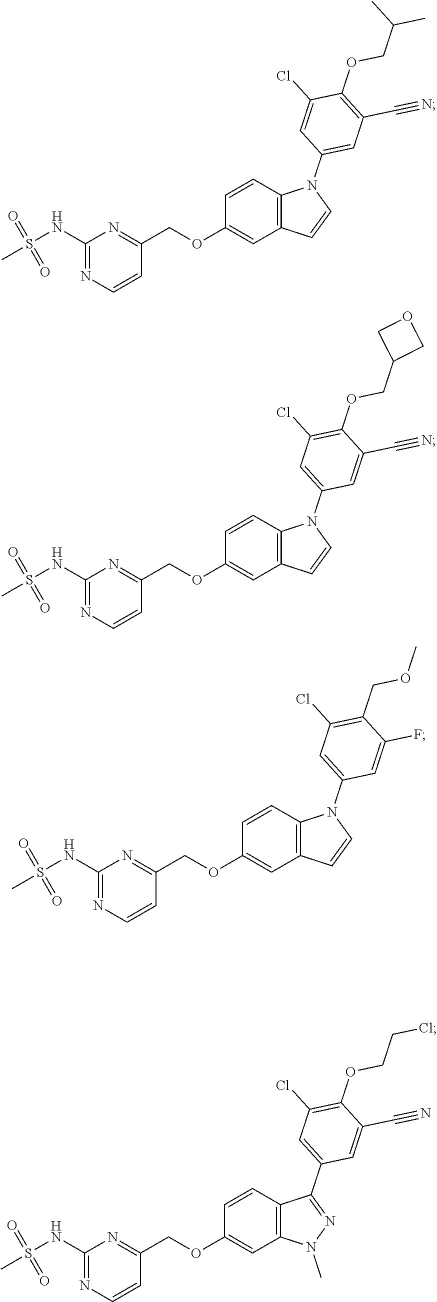 [SMILES: CC(C)COc1c(Cl)cc(-n2ccc3cc(OCc4ccnc(NS(C)(=O)=O)n4)ccc32)cc1C#N ; CS(=O)(=O)Nc1nccc(COc2ccc3c(ccn3-c3cc(Cl)c(OCC4COC4)c(C#N)c3)c2)n1 ; COCc1c(F)cc(-n2ccc3cc(OCc4ccnc(NS(C)(=O)=O)n4)ccc32)cc1Cl ; Cn1nc(-c2cc(Cl)c(OCCCl)c(C#N)c2)c2ccc(OCc3ccnc(NS(C)(=O)=O)n3)cc21] 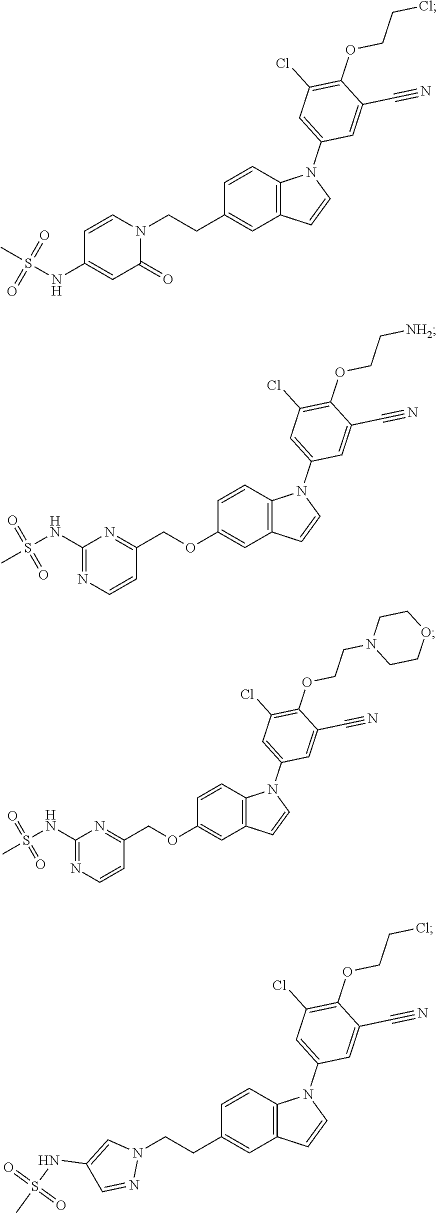 [SMILES: CS(=O)(=O)Nc1ccn(CCc2ccc3c(ccn3-c3cc(Cl)c(OCCCl)c(C#N)c3)c2)c(=O)c1 ; CS(=O)(=O)Nc1nccc(COc2ccc3c(ccn3-c3cc(Cl)c(OCCN)c(C#N)c3)c2)n1 ; CS(=O)(=O)Nc1nccc(COc2ccc3c(ccn3-c3cc(Cl)c(OCCN4CCOCC4)c(C#N)c3)c2)n1 ; CS(=O)(=O)Nc1cnn(CCc2ccc3c(ccn3-c3cc(Cl)c(OCCCl)c(C#N)c3)c2)c1] 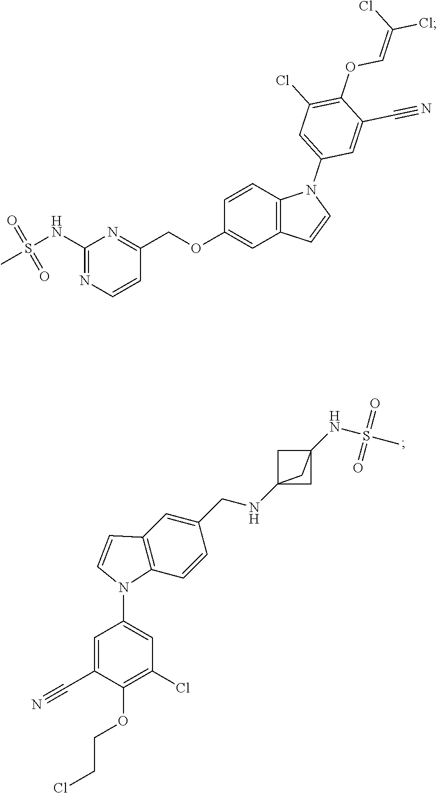 [SMILES: CS(=O)(=O)Nc1nccc(COc2ccc3c(ccn3-c3cc(Cl)c(OC=C(Cl)Cl)c(C#N)c3)c2)n1 ; CS(=O)(=O)NC12CC(NCc3ccc4c(ccn4-c4cc(Cl)c(OCCCl)c(C#N)c4)c3)(C1)C2] 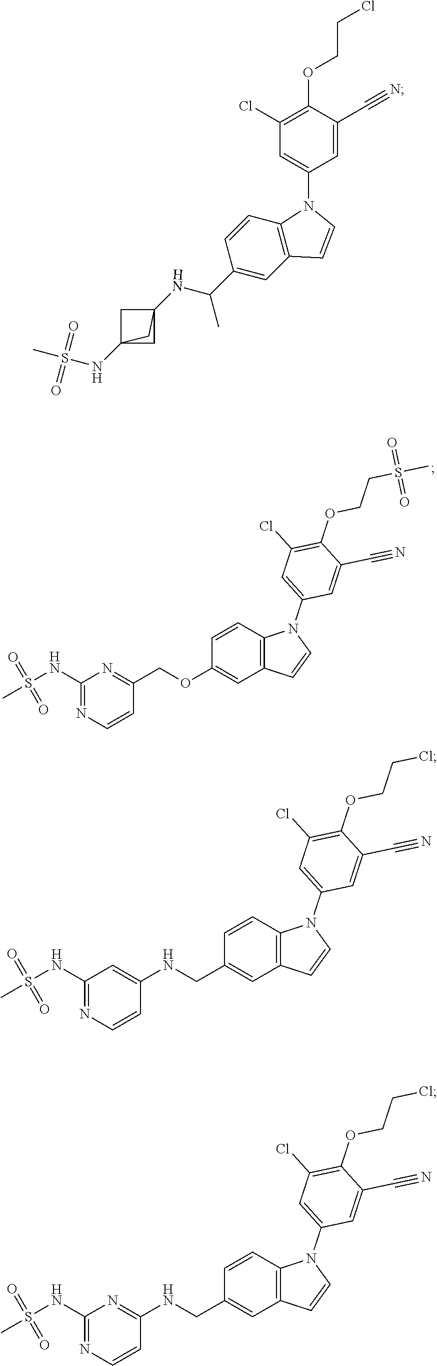 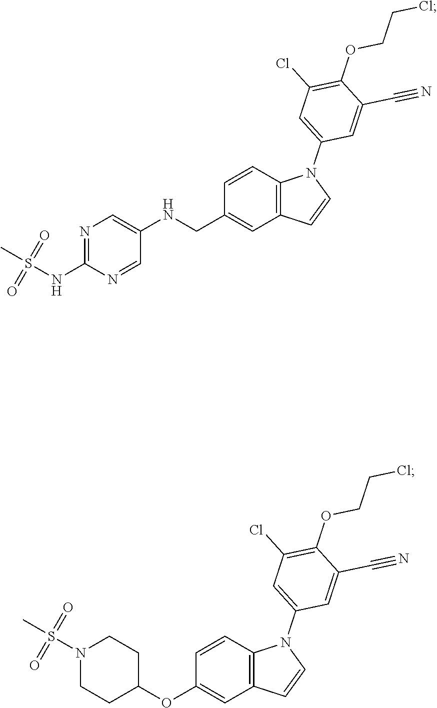 [SMILES: CS(=O)(=O)Nc1ncc(NCc2ccc3c(ccn3-c3cc(Cl)c(OCCCl)c(C#N)c3)c2)cn1 ; CS(=O)(=O)N1CCC(Oc2ccc3c(ccn3-c3cc(Cl)c(OCCCl)c(C#N)c3)c2)CC1] 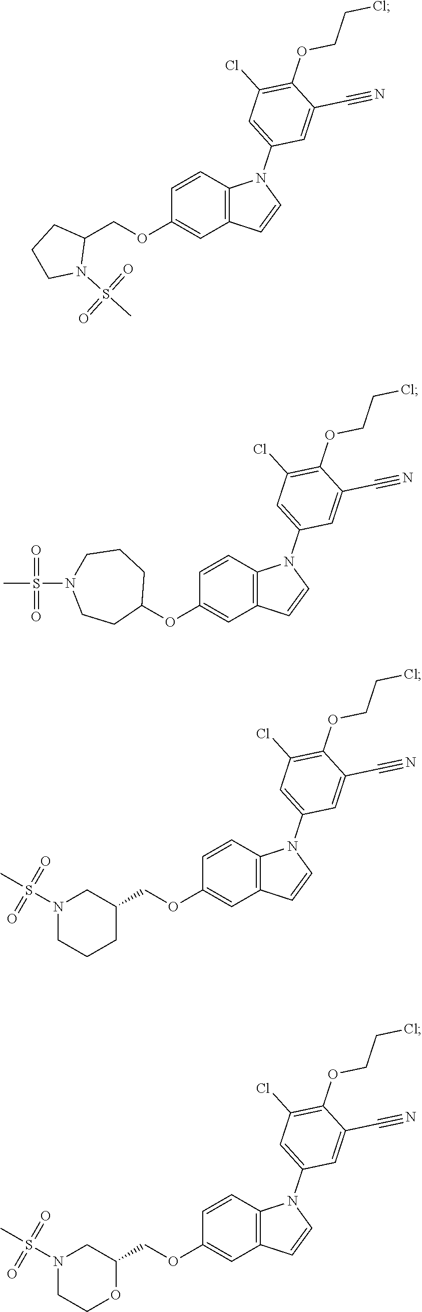 [SMILES: CS(=O)(=O)N1CCCC1COc1ccc2c(ccn2-c2cc(Cl)c(OCCCl)c(C#N)c2)c1 ; CS(=O)(=O)N1CCCC(Oc2ccc3c(ccn3-c3cc(Cl)c(OCCCl)c(C#N)c3)c2)CC1 ; CS(=O)(=O)N1CCC[C@@H](COc2ccc3c(ccn3-c3cc(Cl)c(OCCCl)c(C#N)c3)c2)C1 ; CS(=O)(=O)N1CCO[C@@H](COc2ccc3c(ccn3-c3cc(Cl)c(OCCCl)c(C#N)c3)c2)C1] 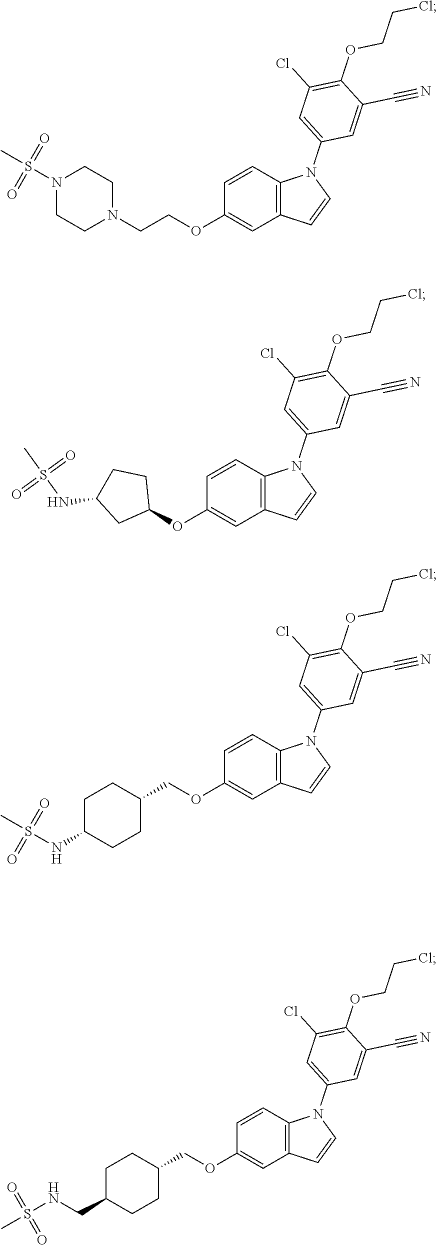 [SMILES: CS(=O)(=O)N1CCN(CCOc2ccc3c(ccn3-c3cc(Cl)c(OCCCl)c(C#N)c3)c2)CC1 ; CS(=O)(=O)N[C@@H]1CC[C@@H](Oc2ccc3c(ccn3-c3cc(Cl)c(OCCCl)c(C#N)c3)c2)C1 ; CS(=O)(=O)N[C@H]1CC[C@@H](COc2ccc3c(ccn3-c3cc(Cl)c(OCCCl)c(C#N)c3)c2)CC1 ; CS(=O)(=O)NC[C@H]1CC[C@H](COc2ccc3c(ccn3-c3cc(Cl)c(OCCCl)c(C#N)c3)c2)CC1] 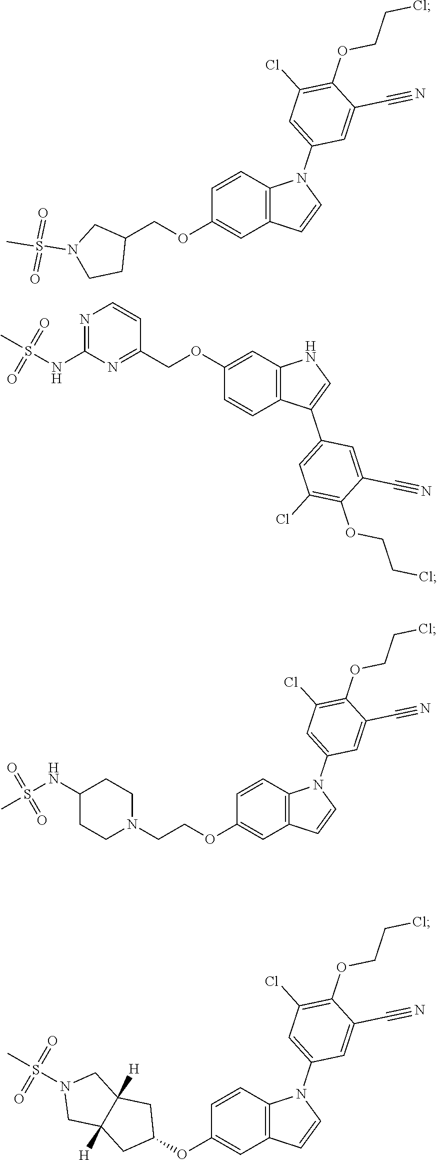 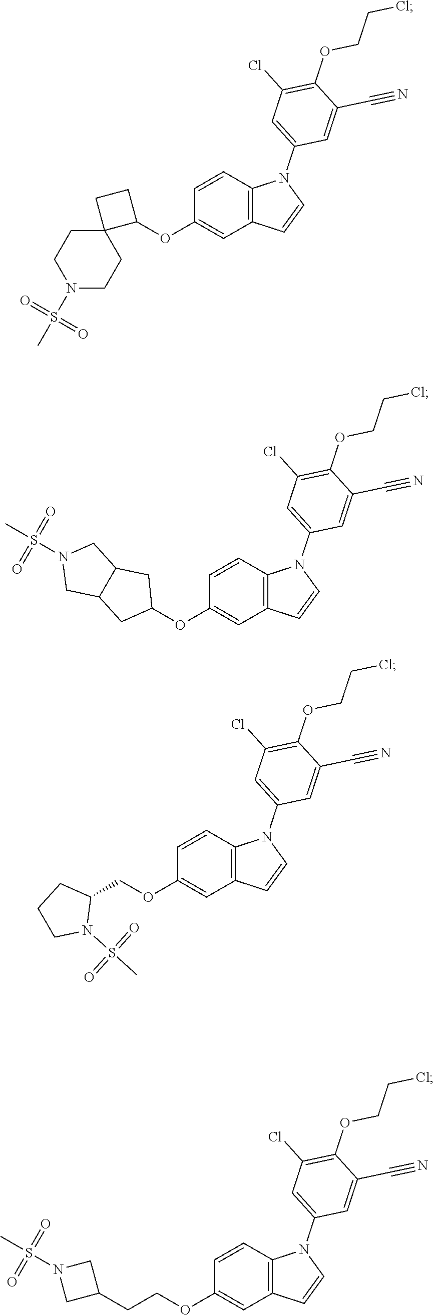 [SMILES: CS(=O)(=O)N1CCC2(CCC2Oc2ccc3c(ccn3-c3cc(Cl)c(OCCCl)c(C#N)c3)c2)CC1 ; CS(=O)(=O)N1CC2CC(Oc3ccc4c(ccn4-c4cc(Cl)c(OCCCl)c(C#N)c4)c3)CC2C1 ; CS(=O)(=O)N1CCC[C@@H]1COc1ccc2c(ccn2-c2cc(Cl)c(OCCCl)c(C#N)c2)c1 ; CS(=O)(=O)N1CC(CCOc2ccc3c(ccn3-c3cc(Cl)c(OCCCl)c(C#N)c3)c2)C1] 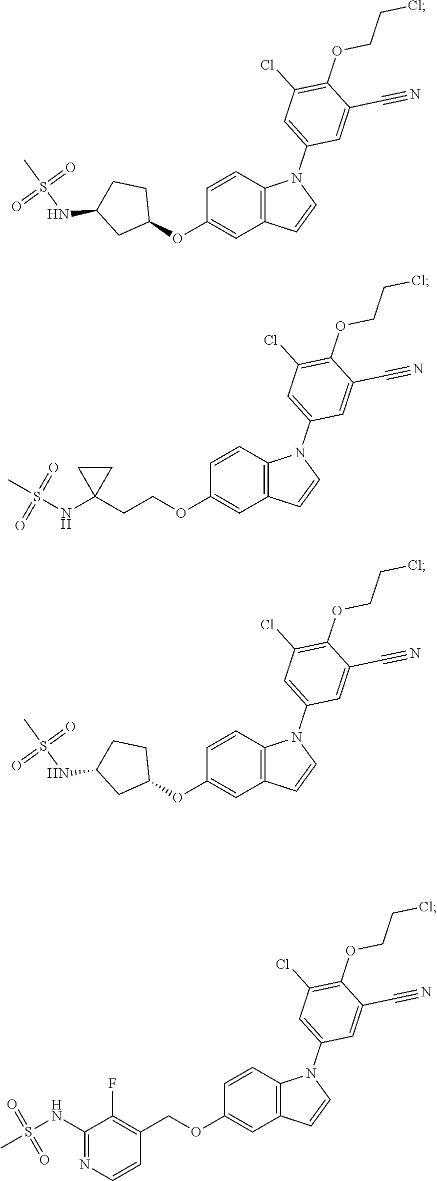 [SMILES: CS(=O)(=O)N[C@H]1CC[C@@H](Oc2ccc3c(ccn3-c3cc(Cl)c(OCCCl)c(C#N)c3)c2)C1 ; CS(=O)(=O)NC1(CCOc2ccc3c(ccn3-c3cc(Cl)c(OCCCl)c(C#N)c3)c2)CC1 ; CS(=O)(=O)N[C@@H]1CC[C@H](Oc2ccc3c(ccn3-c3cc(Cl)c(OCCCl)c(C#N)c3)c2)C1 ; CS(=O)(=O)Nc1nccc(COc2ccc3c(ccn3-c3cc(Cl)c(OCCCl)c(C#N)c3)c2)c1F] 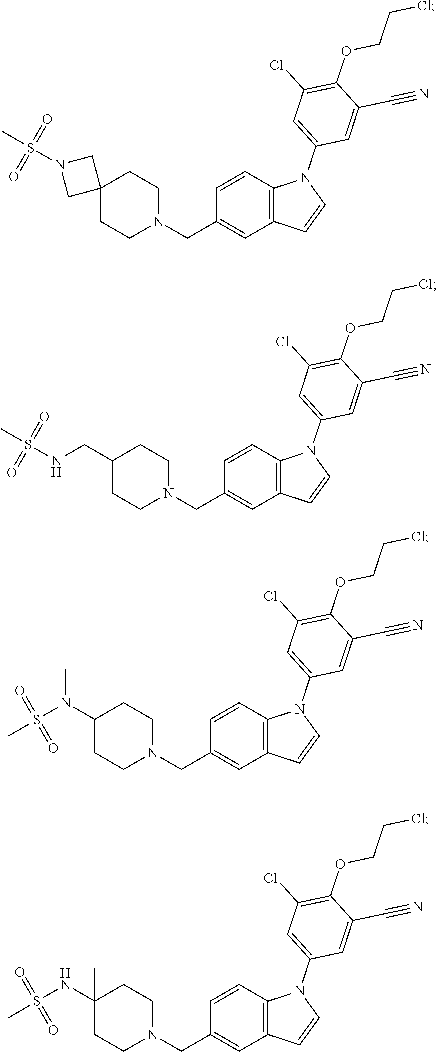 [SMILES: CS(=O)(=O)N1CC2(CCN(Cc3ccc4c(ccn4-c4cc(Cl)c(OCCCl)c(C#N)c4)c3)CC2)C1 ; CS(=O)(=O)NCC1CCN(Cc2ccc3c(ccn3-c3cc(Cl)c(OCCCl)c(C#N)c3)c2)CC1 ; CN(C1CCN(Cc2ccc3c(ccn3-c3cc(Cl)c(OCCCl)c(C#N)c3)c2)CC1)S(C)(=O)=O ; CC1(NS(C)(=O)=O)CCN(Cc2ccc3c(ccn3-c3cc(Cl)c(OCCCl)c(C#N)c3)c2)CC1] 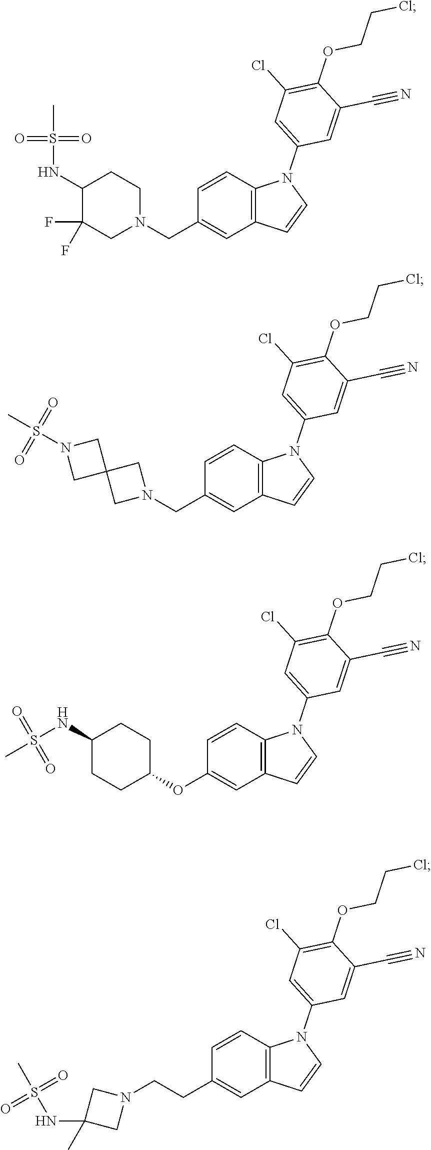 [SMILES: CS(=O)(=O)NC1CCN(Cc2ccc3c(ccn3-c3cc(Cl)c(OCCCl)c(C#N)c3)c2)CC1(F)F ; CS(=O)(=O)N1CC2(CN(Cc3ccc4c(ccn4-c4cc(Cl)c(OCCCl)c(C#N)c4)c3)C2)C1 ; CS(=O)(=O)N[C@H]1CC[C@H](Oc2ccc3c(ccn3-c3cc(Cl)c(OCCCl)c(C#N)c3)c2)CC1 ; CC1(NS(C)(=O)=O)CN(CCc2ccc3c(ccn3-c3cc(Cl)c(OCCCl)c(C#N)c3)c2)C1] 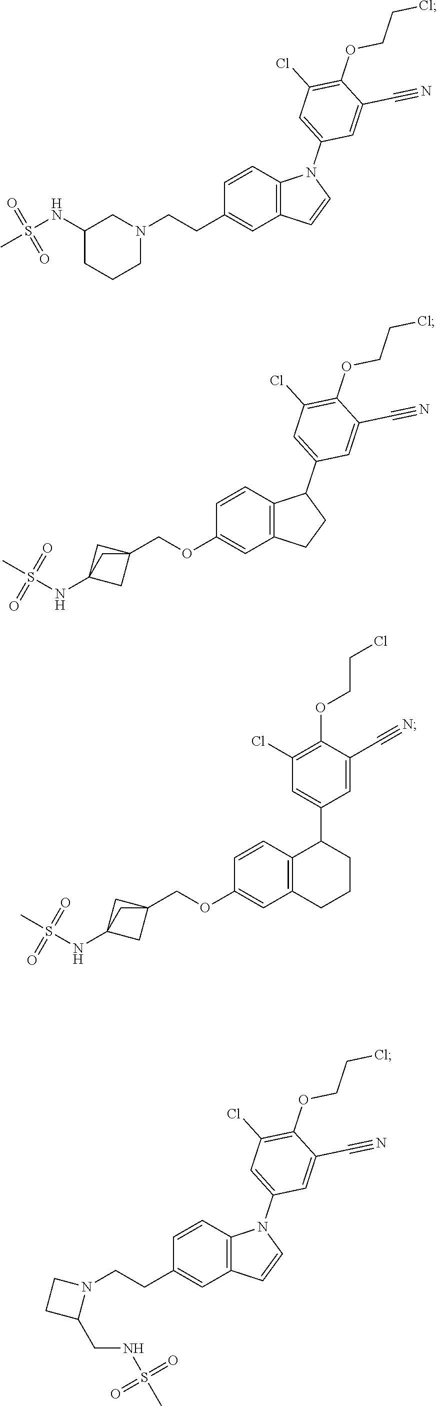 [SMILES: CS(=O)(=O)NC1CCCN(CCc2ccc3c(ccn3-c3cc(Cl)c(OCCCl)c(C#N)c3)c2)C1 ; CS(=O)(=O)NC12CC(COc3ccc4c(c3)CCC4c3cc(Cl)c(OCCCl)c(C#N)c3)(C1)C2 ; CS(=O)(=O)NC12CC(COc3ccc4c(c3)CCCC4c3cc(Cl)c(OCCCl)c(C#N)c3)(C1)C2 ; CS(=O)(=O)NCC1CCN1CCc1ccc2c(ccn2-c2cc(Cl)c(OCCCl)c(C#N)c2)c1] 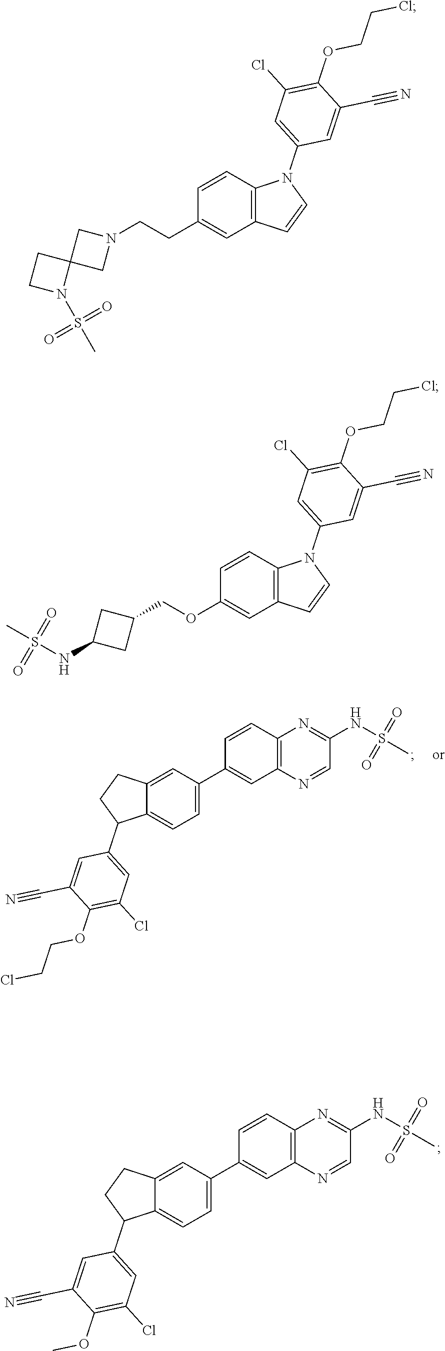 [SMILES: CS(=O)(=O)N1CCC12CN(CCc1ccc3c(ccn3-c3cc(Cl)c(OCCCl)c(C#N)c3)c1)C2 ; CS(=O)(=O)N[C@H]1C[C@H](COc2ccc3c(ccn3-c3cc(Cl)c(OCCCl)c(C#N)c3)c2)C1 ; CS(=O)(=O)Nc1cnc2cc(-c3ccc4c(c3)CCC4c3cc(Cl)c(OCCCl)c(C#N)c3)ccc2n1 ; COc1c(Cl)cc(C2CCc3cc(-c4ccc5nc(NS(C)(=O)=O)cnc5c4)ccc32)cc1C#N]
pharmaceutically acceptable salt thereof.

38. The compound of claim 1 having the structure of: 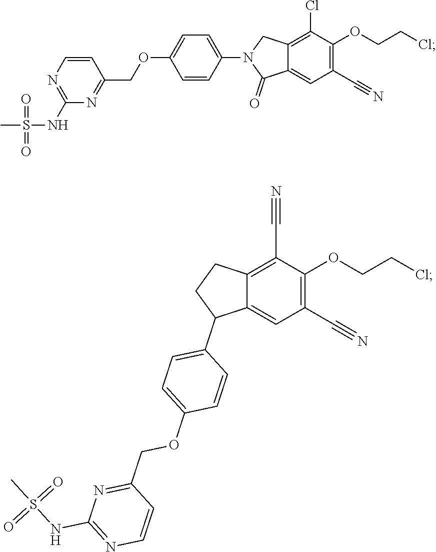 [SMILES: CS(=O)(=O)Nc1nccc(COc2ccc(N3Cc4c(cc(C#N)c(OCCCl)c4Cl)C3=O)cc2)n1 ; CS(=O)(=O)Nc1nccc(COc2ccc(C3CCc4c3cc(C#N)c(OCCCl)c4C#N)cc2)n1] 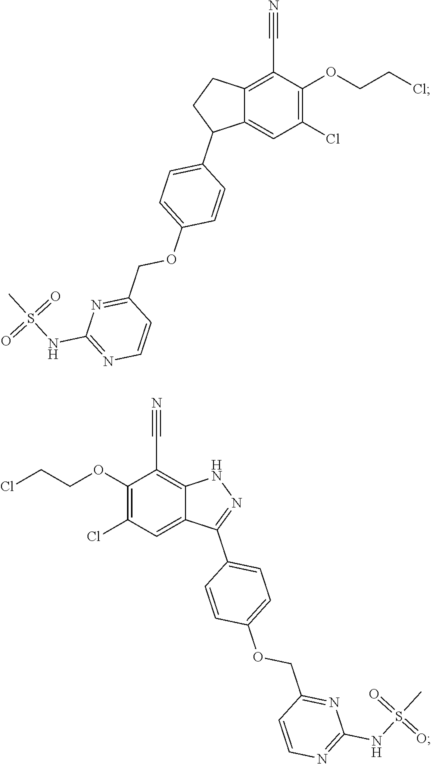 [SMILES: CS(=O)(=O)Nc1nccc(COc2ccc(C3CCc4c3cc(Cl)c(OCCCl)c4C#N)cc2)n1 ; CS(=O)(=O)Nc1nccc(COc2ccc(-c3n[nH]c4c(C#N)c(OCCCl)c(Cl)cc34)cc2)n1] 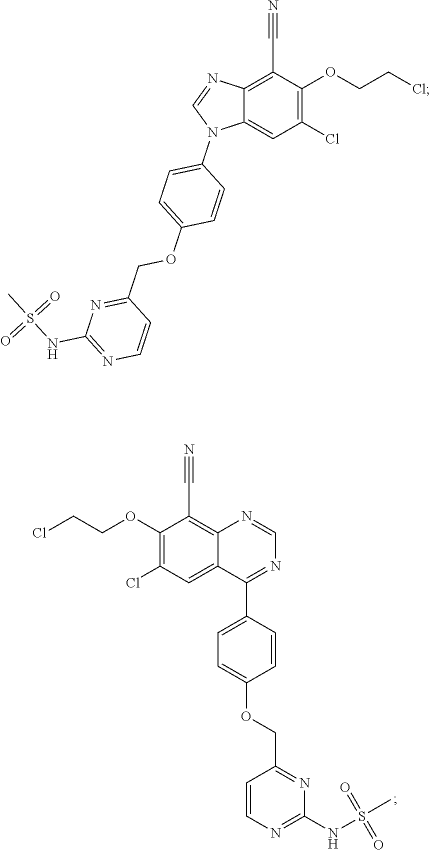 [SMILES: CS(=O)(=O)Nc1nccc(COc2ccc(-n3cnc4c(C#N)c(OCCCl)c(Cl)cc43)cc2)n1 ; CS(=O)(=O)Nc1nccc(COc2ccc(-c3ncnc4c(C#N)c(OCCCl)c(Cl)cc34)cc2)n1] 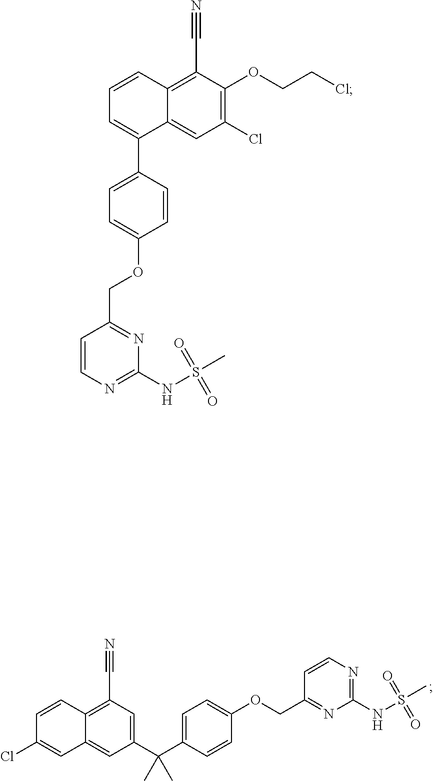 [SMILES: CS(=O)(=O)Nc1nccc(COc2ccc(-c3cccc4c(C#N)c(OCCCl)c(Cl)cc34)cc2)n1 ; CC(C)(c1ccc(OCc2ccnc(NS(C)(=O)=O)n2)cc1)c1cc(C#N)c2ccc(Cl)cc2c1] 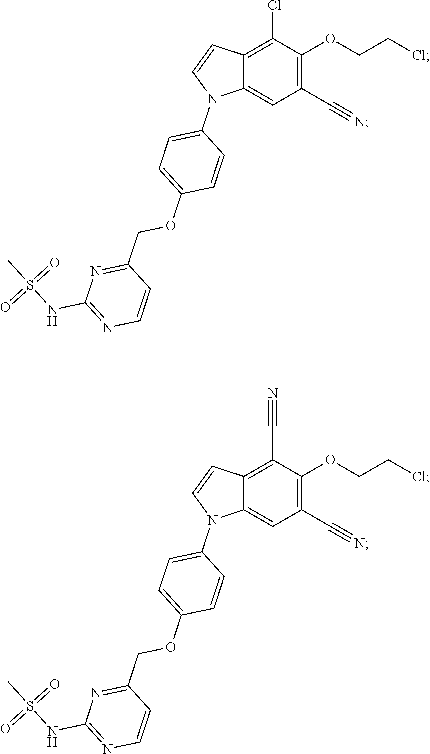 [SMILES: CS(=O)(=O)Nc1nccc(COc2ccc(-n3ccc4c(Cl)c(OCCCl)c(C#N)cc43)cc2)n1 ; CS(=O)(=O)Nc1nccc(COc2ccc(-n3ccc4c(C#N)c(OCCCl)c(C#N)cc43)cc2)n1] 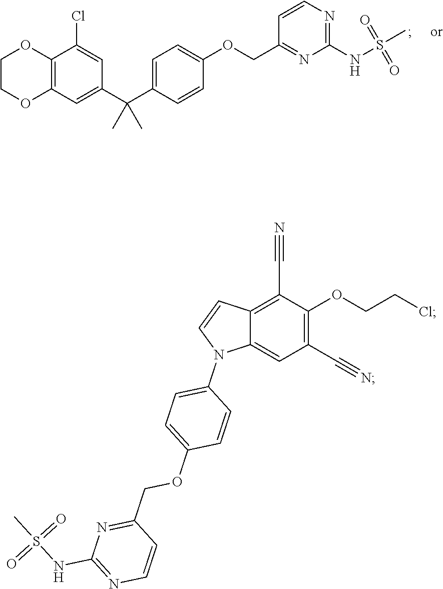 [SMILES: CC(C)(c1ccc(OCc2ccnc(NS(C)(=O)=O)n2)cc1)c1cc(Cl)c2c(c1)OCCO2 ; CS(=O)(=O)Nc1nccc(COc2ccc(-n3ccc4c(C#N)c(OCCCl)c(C#N)cc43)cc2)n1]
or a pharmaceutically acceptable salt thereof.

39. The compound of claim 1, having the structure of formula (G): 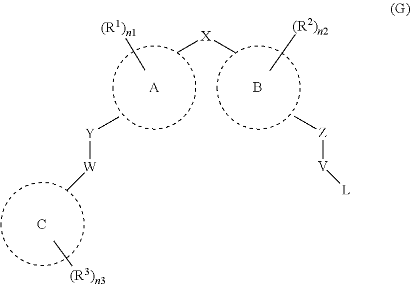
or a pharmaceutically acceptable salt, tautomer, stereoisomer or prodrug thereof, wherein:
A and B are each independently a phenyl ring;
C is a fused or a bridged bicyclic 5- to 15-membered ring selected from aryl, carbocyclyl, heteroaryl, or heterocyclyl;
X is a bond, —CH₂—, —C(CH₃)₂—, or —C(═O)—;
Y is a bond, —CH₂—, —NH—, or —O—;
W is a bond, —CH₂—, —CH₂CH₂—, —C(CH₃)H—, —N(R⁷)CO—, or —CONR⁷—;
Z is a bond —NH—, or —O—;
V is a bond, —CH₂(CR^{8a′}R^{9a′})—, or —(CR^{8a}R^{9a})ₘ—;
L is hydrogen, halogen, —CF₂R¹⁰, —CF₃, —CN, —OR¹⁰, —NR¹¹R¹², or —CONR¹¹R¹²;
R¹ and R² are each independently halogen, —CN, or —CF₃;
at least one R³ is —CH₂NHSO₂CH₃, —CH₂N(CH₃)SO₂CH₃, —NHSO₂CH₃, —NHSO₂CH₂CH₃, —SO₂NH₂, —SO₂CH₃, —NH₂, or —NH(C₁-C₃ alkyl); and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), or —N(CH₃)COO(C₁-C₃ alkyl);
R⁷ is hydrogen or C₁-C₃ alkyl;
R^{8a} and R^{9a} are each independently hydrogen, halogen, or C₁-C₃ alkyl;
R^{8a′} and R^{9a′} taken together form an optionally substituted 3- to 6-membered carbocyclyl or heterocyclyl containing one, two, or three heteroatoms selected from N, S, or O;
R¹⁰ is hydrogen, or C₁-C₃ alkyl;
R¹¹ and R¹² are each independently hydrogen or C₁-C₃ alkyl; or R¹¹ and R¹² taken together form an optionally substituted heterocyclyl which optionally contains one or two additional heteroatoms selected from N, S, or O;
m is 0, 1, or 2;
n1 and n2 are each independently 0, 1, or 2; and
n3 is 1, 2, 3, 4 or 5;
wherein C is not

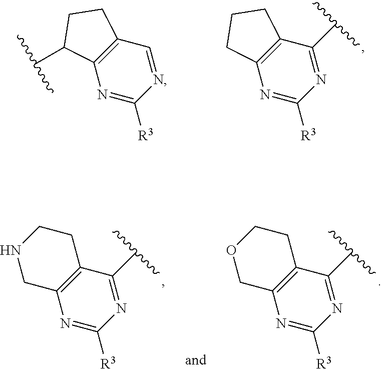

40. The compound of claim 39, wherein C is a 6,6-fused heteroaryl or heterocycle, 5,6-fused heteroaryl or heterocycle, 6,5-fused heteroaryl or heterocycle, or 5,5-fused heteroaryl or heterocycle.

41. The compound of 39, wherein C is

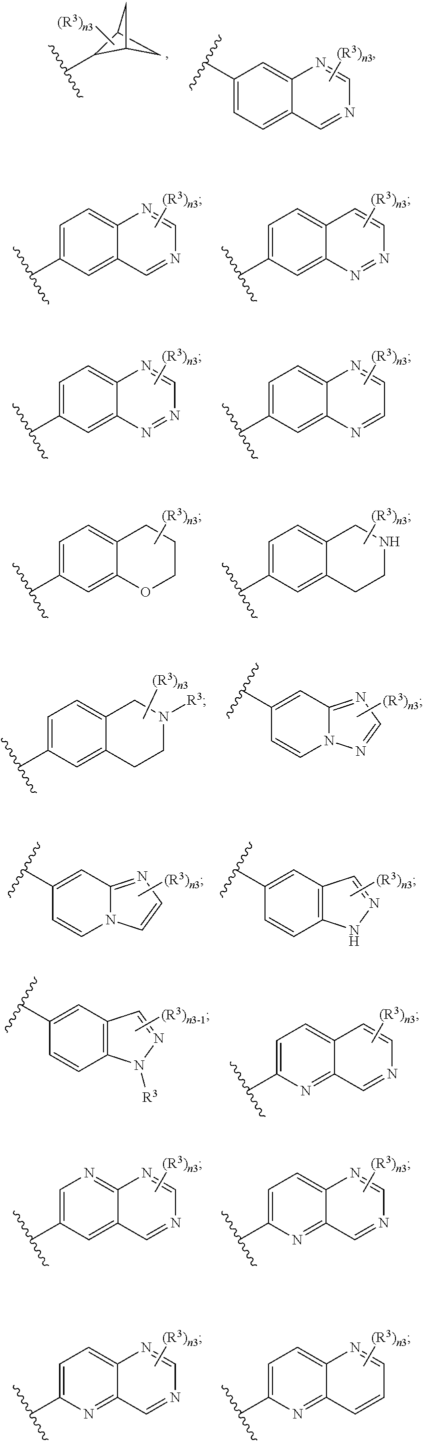

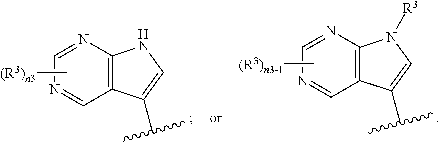

42. The compound of claim 1, having the structure of formula (H): 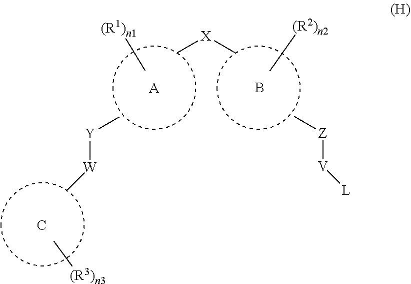
or a pharmaceutically acceptable salt, tautomer, stereoisomer or prodrug thereof, wherein:
A and B are each independently a phenyl or a pyridyl ring;
C is 5- or 6-membered heteroaryl, 4- to 6-membered heterocyclyl, phenyl, or bicyclic 5- or 6-membered carbocycle;
X is a bond, —CH₂—, —C(CH₃)₂—, or —C(═O)—;
Y is a bond, —CH₂—, —NH—, or —O—;
W is —CH₂—, —CH₂CH₂—, —C(CH₃)H—, —N(R⁷)CO—, or —CONR⁷—;
Z is a bond, —NH—, or —O—;
V is a bond, —CH₂(CR^{8a′}R^{9a′})—, or —(CR^{8a}R^{9a})ₘ—;
L is hydrogen, halogen, —CF₂R¹⁰, —CF₃, —CN, —OR¹⁰, —NR¹¹R¹², or —CONR¹¹R¹²;
R¹ and R² are each independently halogen, —CN, or —CF₃;
at least one R³ is —CH₂NHSO₂CH₃, —CH₂N(CH₃)SO₂CH₃, —NHSO₂CH₃, —NHSO₂CH₂CH₃, —SO₂NH₂, —SO₂CH₃, —NH₂, —NH(C₁-C₃ alkyl), —NHCOCF₃ or optionally substituted 5- or 6-membered heterocyclyl or heteroaryl; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁- C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), —N(CH₃)COO(C₁-C₃ alkyl); or optionally substituted 5- or 6-membered heterocyclyl;
R⁷ is hydrogen or C₁-C₃ alkyl;
R^{8a} and R^{9a} are each independently hydrogen, halogen, or C₁-C₃ alkyl;
R^{8a′} and R^{9a′} taken together form an optionally substituted 3- to 6-membered carbocyclyl or heterocyclyl containing one, two, or three heteroatoms selected from N, S, or O;
R¹⁰ is hydrogen, or C₁-C₃ alkyl;
R¹¹ and R¹² are each independently hydrogen or C₁-C₃ alkyl; or R¹¹ and R¹² taken together form an optionally substituted heterocyclyl which optionally contains one or two additional heteroatoms selected from N, S, or O;
m is 0, 1, or 2;
n1 and n2 are each independently 0, 1, or 2; and
n3 is 1, 2, 3, 4 or 5;
wherein:
a) at least one R³ is optionally substituted 4- to 6-membered heterocyclyl or heteroaryl, provided that 5- or 6-membered heterocyclyl is not 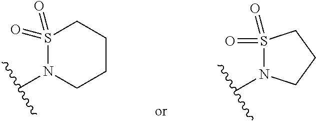 [SMILES: *N1CCCCS1(=O)=O ; *N1CCCS1(=O)=O]
when R³ is pyrimidyl; or
b) Z is a bond; V is a bond; L is —NR¹¹R¹²; and R¹¹ and R¹², taken together with the nitrogen atom to which it is attached, form an optionally substituted heterocyclic ring.

43.-48. (canceled)

49. The compound of claim 39, wherein:
Z is a bond —NH—, or —O—; and
V is —CH₂— and L is halogen, —CHCl₂, —CCl₃, or —CF₃; or
V is —CH₂CH₂— and L is halogen.

50. The compound of claim 1 having the structure of formula (J): 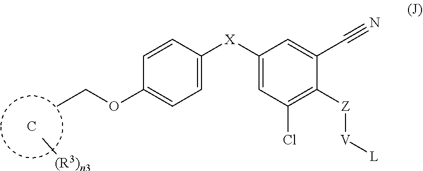
or a pharmaceutically acceptable salt, tautomer, stereoisomer or prodrug thereof, wherein:
C is pyrimidyl;
X is a bond, —CH₂— or —C(CH₃)₂—;
Z is —O— and —V-L is —CH₂CH(OH)CH₃, —CH₂CH(OH)CH₂Cl, or —CH₂CH₂CH₂Cl; or
Z is —NH—, V is —(CR^{8a}R^{9a})ₘ—, and L is hydrogen or halogen;
at least one R³ is —CH₂NHSO₂CH₃, —CH₂N(CH₃)SO₂CH₃, —NHSO₂CH₃, —NHSO₂CH₂CH₃, —SO₂NH₂, —SO₂CH₃, —NH₂, —NH(C₁-C₃ alkyl), —NHCOCF₃ or optionally substituted 5- or 6-membered heterocyclyl or heteroaryl; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁- C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), —N(CH₃)COO(C₁-C₃ alkyl); or optionally substituted 5- or 6-membered heterocyclyl;
R^{8a} and R^{9a} are each independently hydrogen, halogen, or C₁-C₃ alkyl;
m is 1, or 2; and
n3 is 1, 2, 3, 4 or 5.

51. The compound of claim 1 having the structure of formula (K): 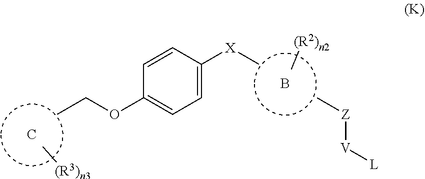
or a pharmaceutically acceptable salt, tautomer, stereoisomer or prodrug thereof, wherein:
B is pyridyl;
C is pyrimidyl;
X is a bond, —CH₂—, —C(CH₃)₂—, or —C(═O)—;
Z is a bond, —NH—, or —O—;
V is a bond, —CH₂(CR^{8a′}R^{9a′})—, or —(CR^{8a}R^{9a})ₘ—;
L is hydrogen, halogen, —CF₂R¹⁰, —CF₃, —CN, —OR¹⁰, —NR¹¹R¹², or —CONR¹¹R¹²;
R² is each independently halogen, —CN, or —CF₃;
at least one R³ is —CH₂NHSO₂CH₃, —CH₂N(CH₃)SO₂CH₃, —NHSO₂CH₃, —NHSO₂CH₂CH₃, —SO₂NH₂, —SO₂CH₃, —NH₂, —NH(C₁-C₃ alkyl), —NHCOCF₃ or optionally substituted 5- or 6-membered heterocyclyl or heteroaryl; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁-C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁- C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), —N(CH₃)COO(C₁-C₃ alkyl); or optionally substituted 5- or 6-membered heterocyclyl;
R^{8a} and R^{9a} are each independently hydrogen, halogen, or C₁-C₃ alkyl;
R^{8a′} and R^{9a′} taken together form an optionally substituted 3- to 6-membered carbocyclyl or heterocyclyl containing one, two, or three heteroatoms selected from N, S, or O;
R¹⁰ is hydrogen, or C₁-C₃ alkyl;
R¹¹ and R¹² are each independently hydrogen or C₁-C₃ alkyl; or R¹¹ and R¹² taken together form an optionally substituted heterocyclyl which optionally contains one or two additional heteroatoms selected from N, S, or O;
m is 1, 2, or 3;
n2 is 0, 1, or 2; and
n3 is 1, 2, 3, 4 or 5.

52. (canceled)

53. The compound of claim 1 having the structure of formula (C): 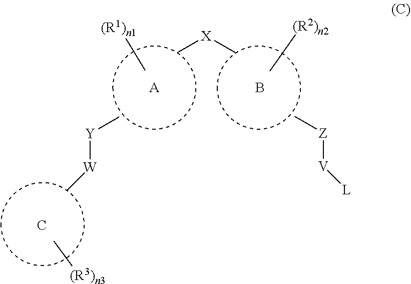
or a pharmaceutically acceptable salt, tautomer, stereoisomer or prodrug thereof, wherein:
A and B are each independently a phenyl ring;
C is a heteroaryl ring;
X is a bond, —CH₂—, —C(CH₃)₂—, or —C(═O)—;
Y is a bond or —O—;
W is —CH₂— or —C(CH₃)H—;
Z is a bond or —O—;
V is a phenylene;
L is halogen, —CHCl₂, —CCl₃, or —CF₃; or
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —NHCOO(C₁-C₃ alkyl), —N(CH₃)CO(C₁-C₃ alkyl), or —N(CH₃)COO(C₁-C₃ alkyl);
n1 and n2 are each independently 0, 1, or 2; and
n3 is 1, 2, 3, 4 or 5.

54. The compound of claim 1 having the structure of formula (E): 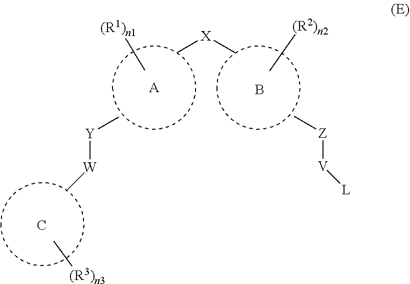
or a pharmaceutically acceptable salt, tautomer, stereoisomer or prodrug thereof, wherein:
A is a 5- or 6-membered heterocyclyl or heteroaryl;
B is phenyl;
C is a heteroaryl ring;
X is a bond, —CH₂—, —C(CH₃)₂—, or —C(═O)—;
Y is a bond or —O—;
W is —CH₂— or —C(CH₃)H—;
Z is a bond or —O—;
V is —CH₂— and L is halogen, —CHCl₂, —CCl₃, or —CF₃; or
V is —CH₂CH₂— and L is halogen;
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —NHCOO(C₁-C₃ alkyl), —N(CH₃)CO(C₁-C₃ alkyl), or —N(CH₃)COO(C₁-C₃ alkyl);
n1 and n2 are each independently 0, 1, or 2; and
n3 is 1, 2, 3, 4 or 5;
wherein the compound is not

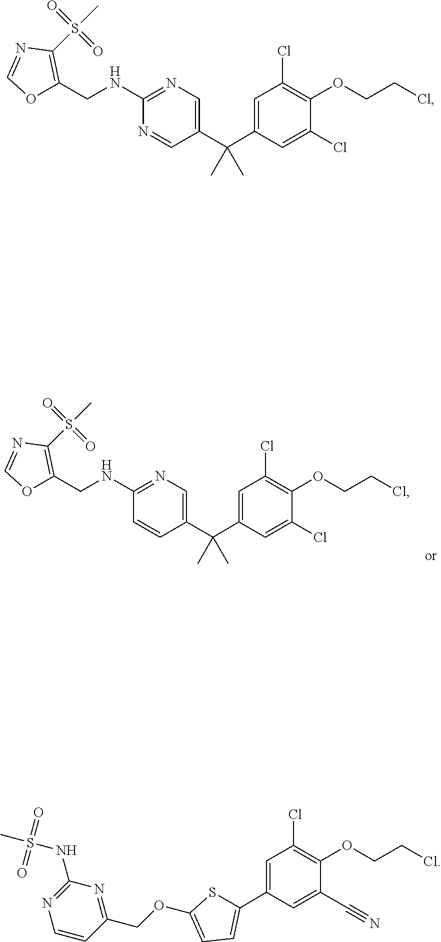 [SMILES: CC(C)(c1cnc(NCc2ocnc2S(C)(=O)=O)nc1)c1cc(Cl)c(OCCCl)c(Cl)c1 ; CC(C)(c1ccc(NCc2ocnc2S(C)(=O)=O)nc1)c1cc(Cl)c(OCCCl)c(Cl)c1 ; CS(=O)(=O)Nc1nccc(COc2ccc(-c3cc(Cl)c(OCCCl)c(C#N)c3)s2)n1]

55.-56. (canceled)

57. The compound of claim 1 having the structure of formula (E-I): 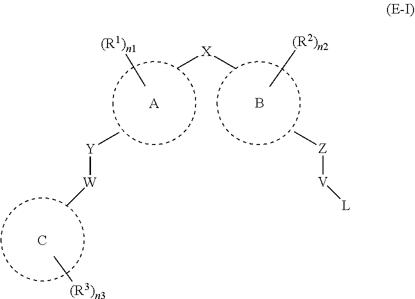
or a pharmaceutically acceptable salt, tautomer, stereoisomer or prodrug thereof, wherein:
A is a 5- or 6-membered heterocyclyl or heteroaryl;
B is phenyl;
C is a 6- to 12-membered heteroaryl ring;
X is a bond, —CH₂—, —CH(CH₃)—, —C(CH₃)₂—, or —C(═O)—;
Y is a bond, —O—, or —NH—;
W is a bond, —CH₂— or —C(CH₃)H—;
Z is a bond —NH—, or —O—;
V is —CH₂— and L is halogen, —CHCl₂, —CCl₃, or —CF₃; or
V is —CH₂CH₂— and L is halogen;
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —NHCOO(C₁-C₃ alkyl), —N(CH₃)CO(C₁-C₃ alkyl), or —N(CH₃)COO(C₁-C₃ alkyl);
n1 and n2 are each independently 0, 1, or 2; and
n3 is 1, 2, 3, or 4;
wherein A is not thiophene.

58.-60. (canceled)

61. The compound of claim 1 having the structure of formula (F): 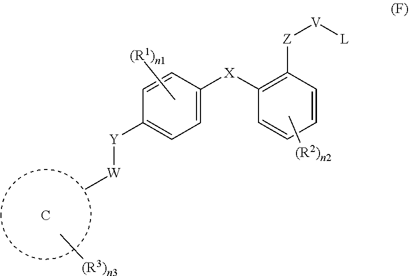
or a pharmaceutically acceptable salt, tautomer, stereoisomer or prodrug thereof, wherein:
A is a 5- or 6-membered heterocyclyl or heteroaryl;
B is phenyl;
C is a pyrimidine, triazine, or thiophene ring;
X is a bond, —CH₂—, —C(CH₃)₂—, or —C(═O)—;
Y is a bond or —O—;
W is —CH₂— or —C(CH₃)H—;
Z is a bond or —O—;
V is —CH₂— and L is halogen, —CHCl₂, —CCl₃, or —CF₃; or
V is —CH₂CH₂— and L is halogen;
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —NHCOO(C₁-C₃ alkyl), —N(CH₃)CO(C₁-C₃ alkyl), or —N(CH₃)COO(C₁-C₃ alkyl);
n1 and n2 are each independently 0, 1, or 2; and
n3 is 1, 2, 3, 4 or 5.

62. (canceled)

63. The compound of claim 39, wherein at least one R³ is —CH₂NHSO₂CH₃, —CH₂N(CH₃)SO₂CH₃, —NHSO₂CH₃, —NHSO₂CH₂CH₃, —SO₂NH₂, —SO₂CH₃, —NH₂, —NH(C₁-C₃ alkyl), or —NHCOCF₃; and the other R³ is, if present, —CN, —CF₃, C₁-C₃ alkyl, C₂-C₃ alkenyl, C₂-C₃ alkynyl, C₁-C₃ alkoxy, oxo, —S(C₁-C₃ alkyl), —SO₂(C₁- C₃ alkyl), —NH₂, —NH(C₁-C₃ alkyl), —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —N(CH₃)CO(C₁-C₃ alkyl), —NHCOCF₃, —N(CH₃)COCF₃, —NHCOO(C₁-C₃ alkyl), —N(CH₃)COO(C₁-C₃ alkyl); or optionally substituted 5- or 6-membered heterocyclyl.

64.-65. (canceled)

66. The compound of claim 1 having the structure: 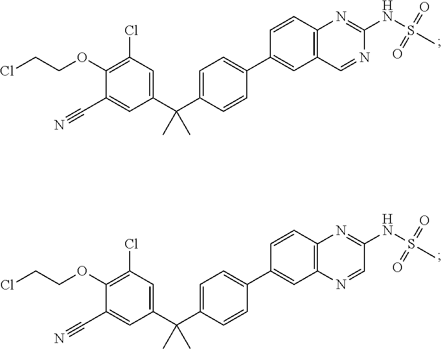 [SMILES: CC(C)(c1ccc(-c2ccc3nc(NS(C)(=O)=O)ncc3c2)cc1)c1cc(Cl)c(OCCCl)c(C#N)c1 ; CC(C)(c1ccc(-c2ccc3nc(NS(C)(=O)=O)cnc3c2)cc1)c1cc(Cl)c(OCCCl)c(C#N)c1] 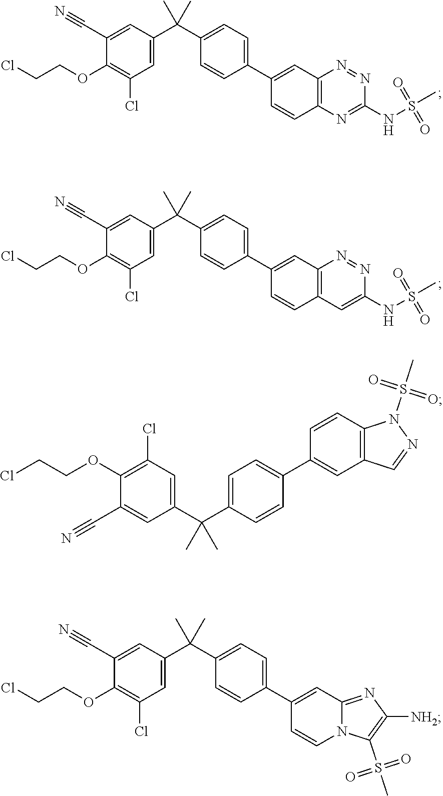 [SMILES: CC(C)(c1ccc(-c2ccc3nc(NS(C)(=O)=O)nnc3c2)cc1)c1cc(Cl)c(OCCCl)c(C#N)c1 ; CC(C)(c1ccc(-c2ccc3cc(NS(C)(=O)=O)nnc3c2)cc1)c1cc(Cl)c(OCCCl)c(C#N)c1 ; CC(C)(c1ccc(-c2ccc3c(cnn3S(C)(=O)=O)c2)cc1)c1cc(Cl)c(OCCCl)c(C#N)c1 ; CC(C)(c1ccc(-c2ccn3c(S(C)(=O)=O)c(N)nc3c2)cc1)c1cc(Cl)c(OCCCl)c(C#N)c1] 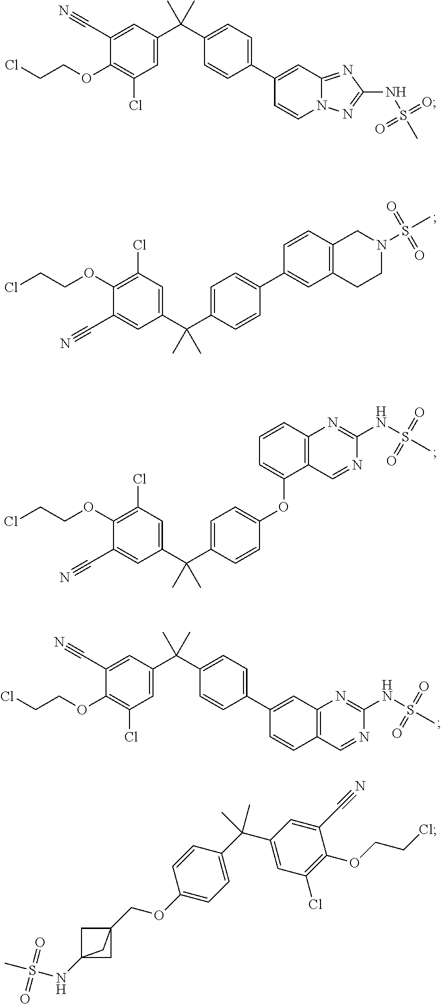 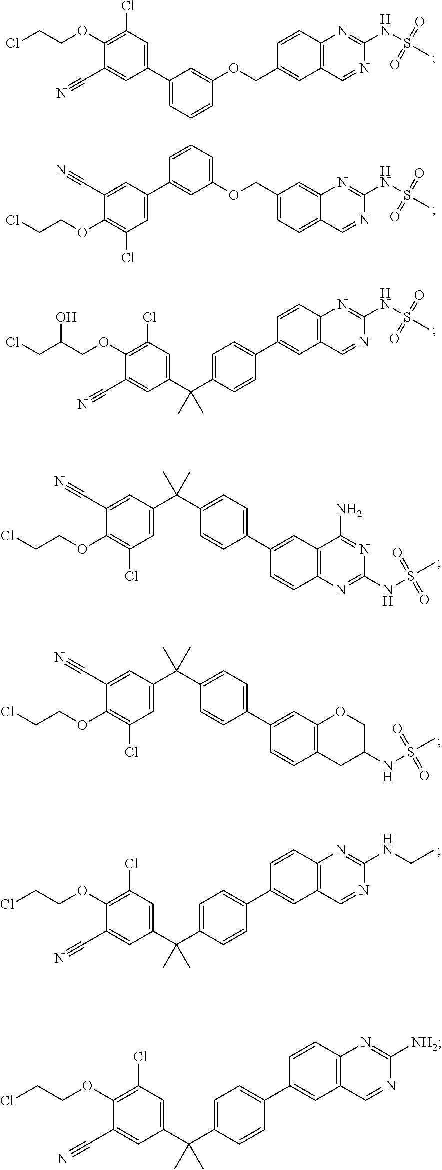 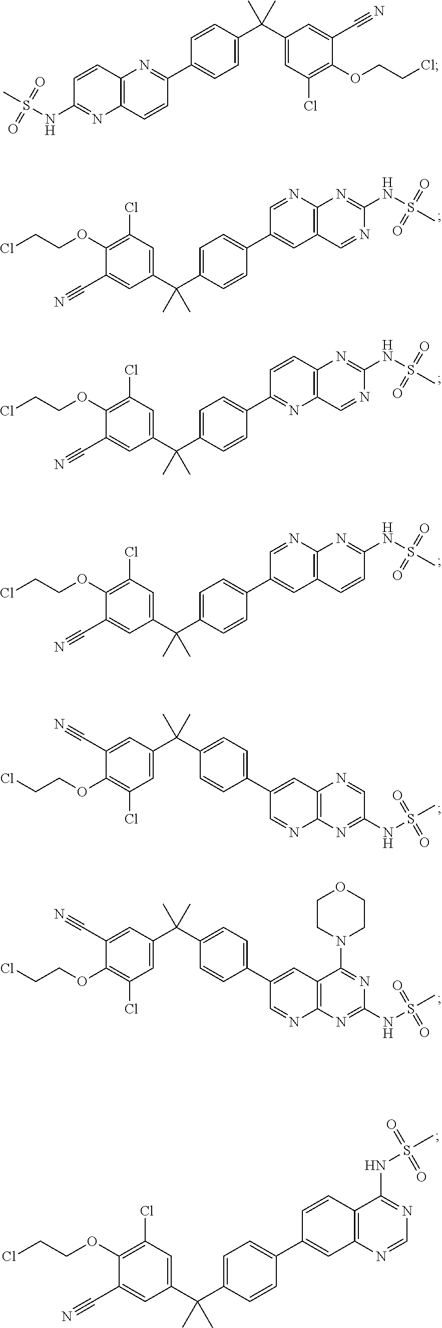 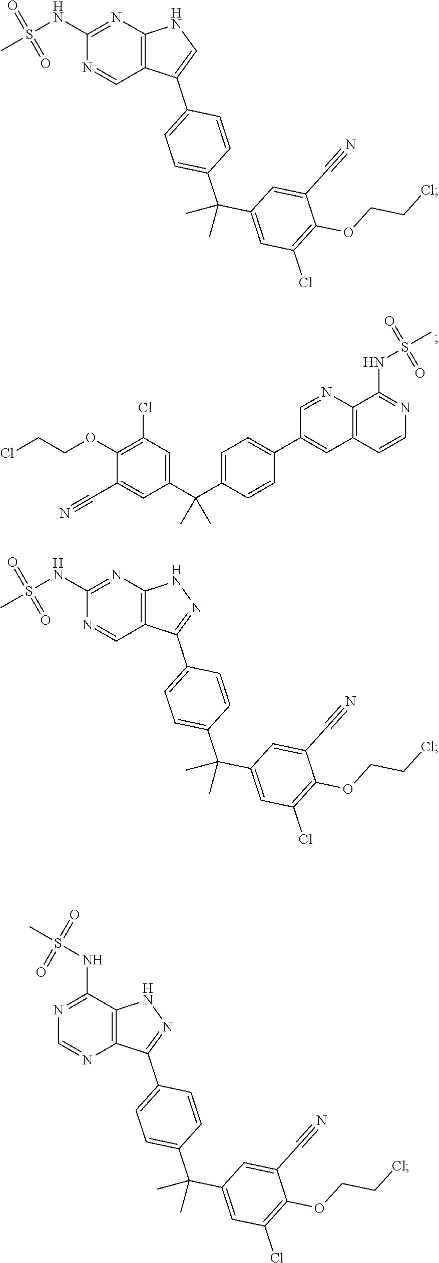 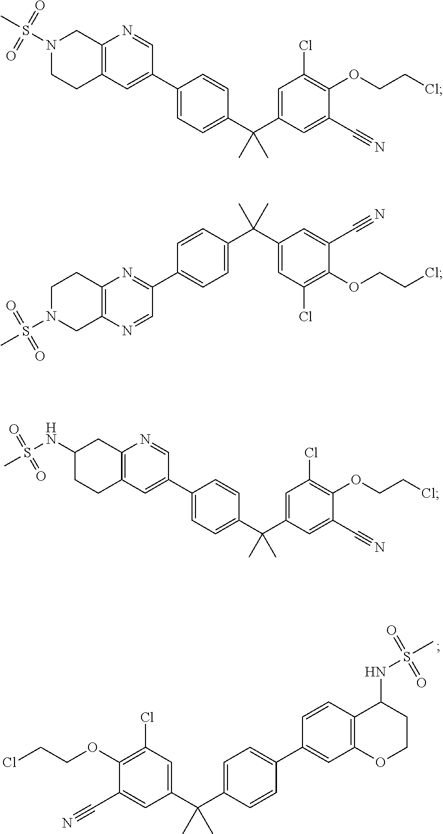 [SMILES: CC(C)(c1ccc(-c2cnc3c(c2)CCN(S(C)(=O)=O)C3)cc1)c1cc(Cl)c(OCCCl)c(C#N)c1 ; CC(C)(c1ccc(-c2cnc3c(n2)CCN(S(C)(=O)=O)C3)cc1)c1cc(Cl)c(OCCCl)c(C#N)c1 ; CC(C)(c1ccc(-c2cnc3c(c2)CCC(NS(C)(=O)=O)C3)cc1)c1cc(Cl)c(OCCCl)c(C#N)c1 ; CC(C)(c1ccc(-c2ccc3c(c2)OCCC3NS(C)(=O)=O)cc1)c1cc(Cl)c(OCCCl)c(C#N)c1] 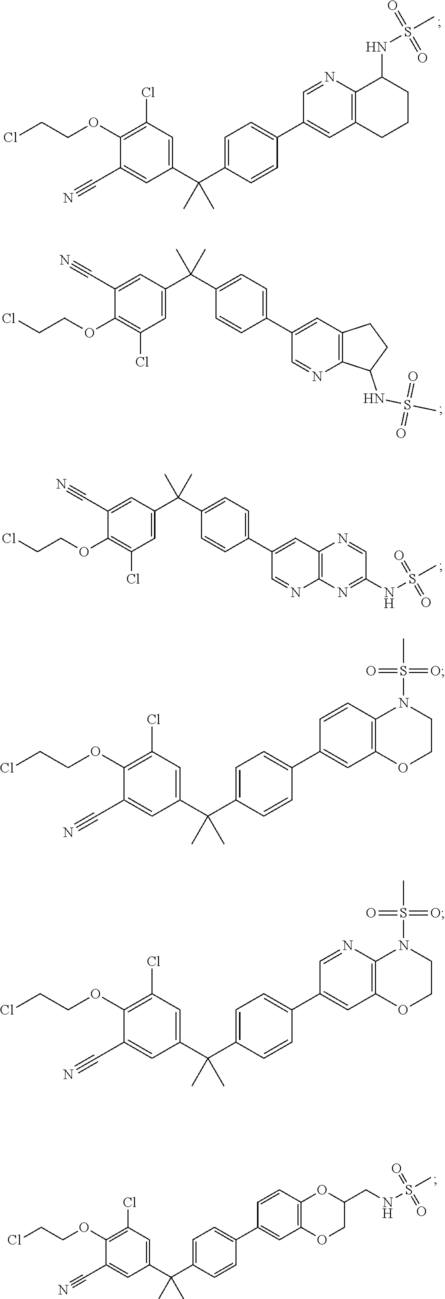 [SMILES: CC(C)(c1ccc(-c2cnc3c(c2)CCCC3NS(C)(=O)=O)cc1)c1cc(Cl)c(OCCCl)c(C#N)c1 ; CC(C)(c1ccc(-c2cnc3c(c2)CCC3NS(C)(=O)=O)cc1)c1cc(Cl)c(OCCCl)c(C#N)c1 ; CC(C)(c1ccc(-c2cnc3nc(NS(C)(=O)=O)cnc3c2)cc1)c1cc(Cl)c(OCCCl)c(C#N)c1 ; CC(C)(c1ccc(-c2ccc3c(c2)OCCN3S(C)(=O)=O)cc1)c1cc(Cl)c(OCCCl)c(C#N)c1 ; CC(C)(c1ccc(-c2cnc3c(c2)OCCN3S(C)(=O)=O)cc1)c1cc(Cl)c(OCCCl)c(C#N)c1 ; CC(C)(c1ccc(-c2ccc3c(c2)OCC(CNS(C)(=O)=O)O3)cc1)c1cc(Cl)c(OCCCl)c(C#N)c1] 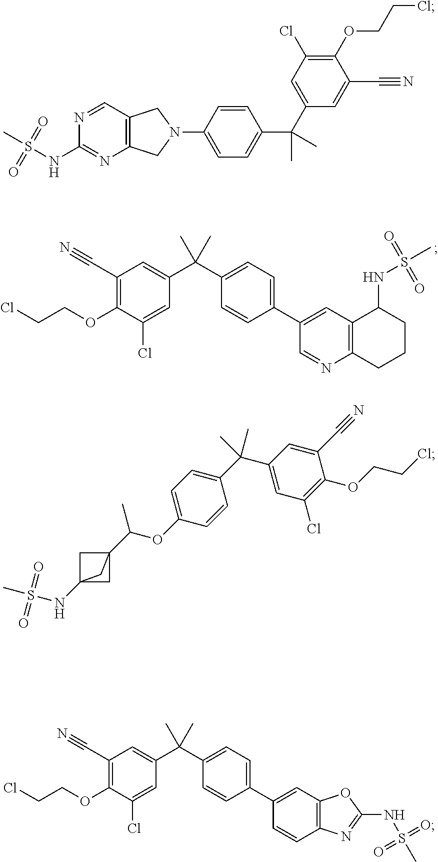 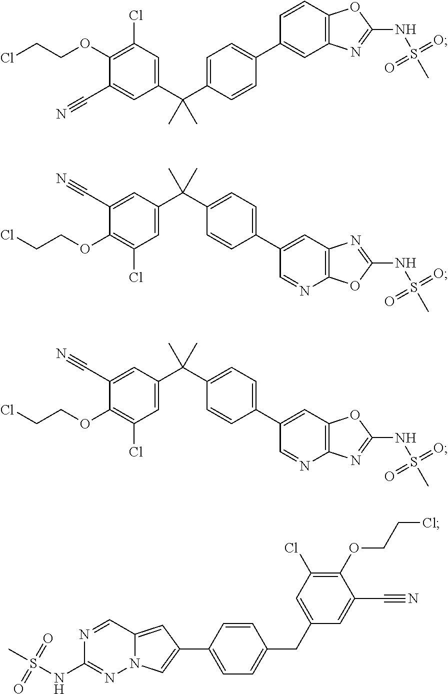 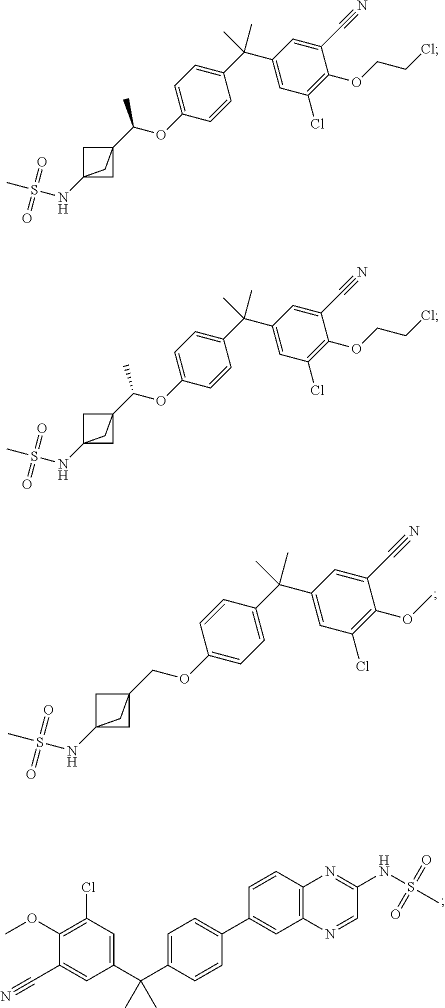 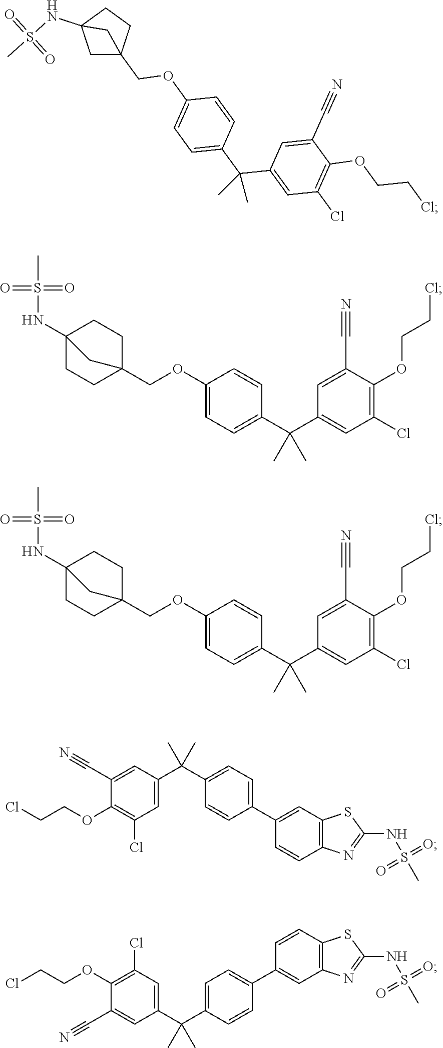 [SMILES: CC(C)(c1ccc(OCC23CCC(NS(C)(=O)=O)(C2)C3)cc1)c1cc(Cl)c(OCCCl)c(C#N)c1 ; CC(C)(c1ccc(OCC23CCC(NS(C)(=O)=O)(CC2)C3)cc1)c1cc(Cl)c(OCCCl)c(C#N)c1 ; CC(C)(c1ccc(OCC23CCC(NS(C)(=O)=O)(CC2)C3)cc1)c1cc(Cl)c(OCCCl)c(C#N)c1 ; CC(C)(c1ccc(-c2ccc3nc(NS(C)(=O)=O)sc3c2)cc1)c1cc(Cl)c(OCCCl)c(C#N)c1 ; CC(C)(c1ccc(-c2ccc3sc(NS(C)(=O)=O)nc3c2)cc1)c1cc(Cl)c(OCCCl)c(C#N)c1] 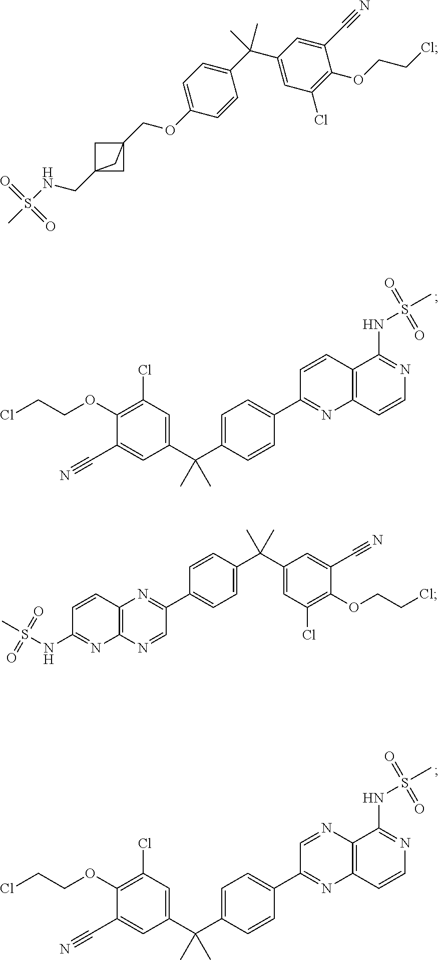 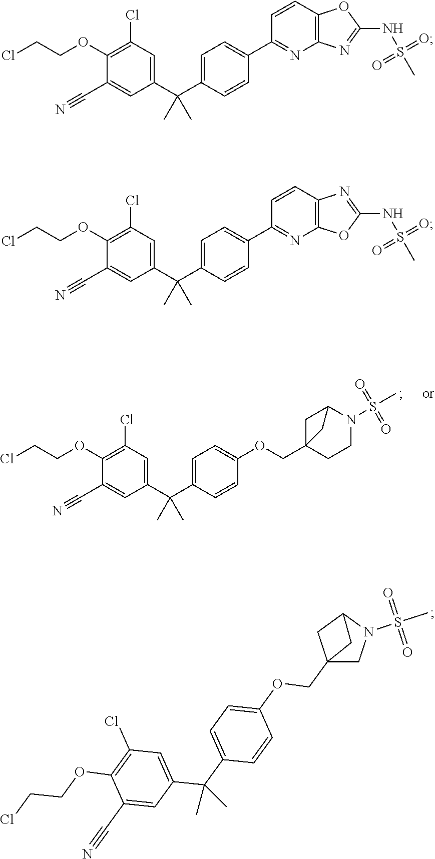 [SMILES: CC(C)(c1ccc(-c2ccc3oc(NS(C)(=O)=O)nc3n2)cc1)c1cc(Cl)c(OCCCl)c(C#N)c1 ; CC(C)(c1ccc(-c2ccc3nc(NS(C)(=O)=O)oc3n2)cc1)c1cc(Cl)c(OCCCl)c(C#N)c1 ; CC(C)(c1ccc(OCC23CCN(S(C)(=O)=O)C(C2)C3)cc1)c1cc(Cl)c(OCCCl)c(C#N)c1 ; CC(C)(c1ccc(OCC23CC(C2)N(S(C)(=O)=O)C3)cc1)c1cc(Cl)c(OCCCl)c(C#N)c1]
or a pharmaceutically acceptable salt thereof.

67. The compound of claim 1 having the structure: 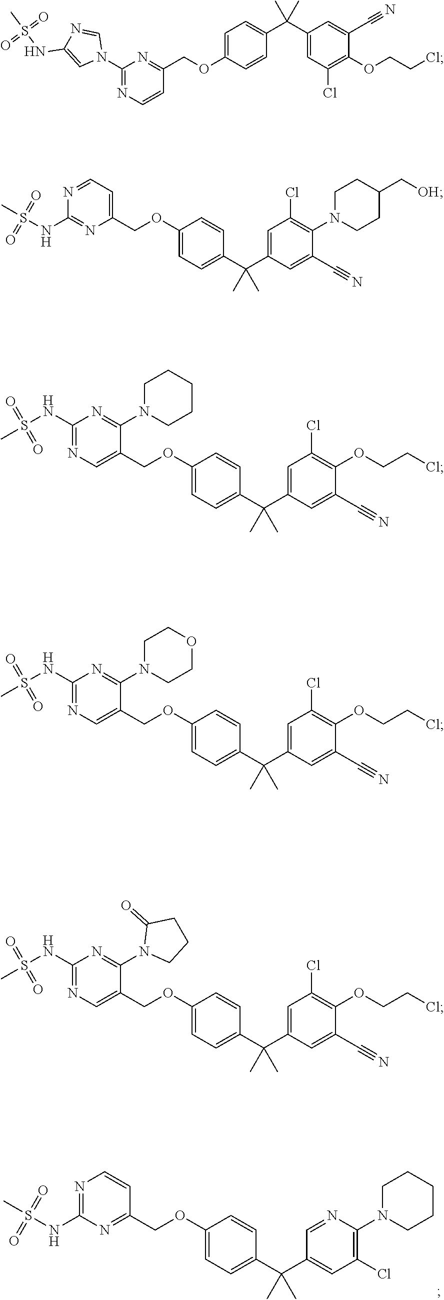 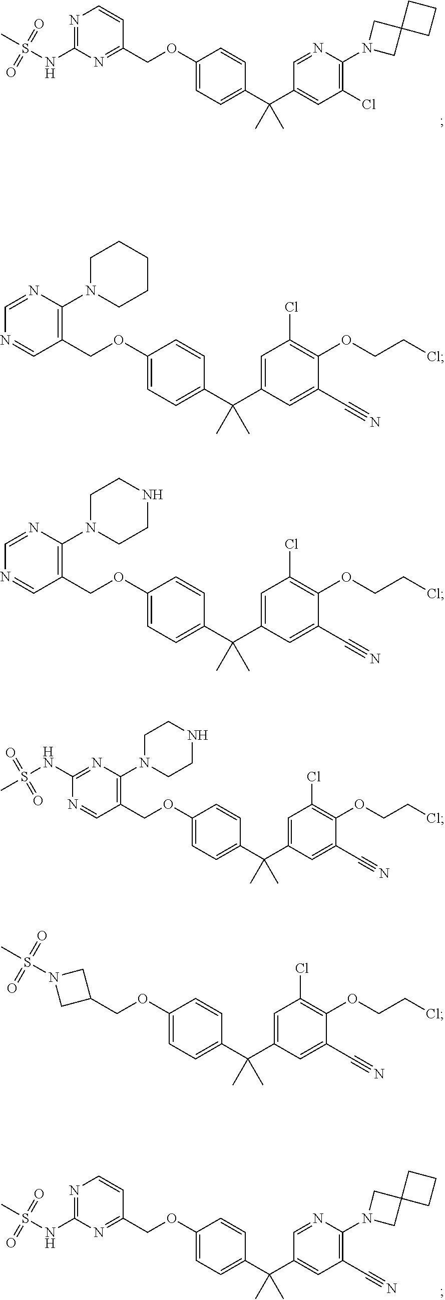 [SMILES: CC(C)(c1ccc(OCc2ccnc(NS(C)(=O)=O)n2)cc1)c1cnc(N2CC3(CCC3)C2)c(Cl)c1 ; CC(C)(c1ccc(OCc2cncnc2N2CCCCC2)cc1)c1cc(Cl)c(OCCCl)c(C#N)c1 ; CC(C)(c1ccc(OCc2cncnc2N2CCNCC2)cc1)c1cc(Cl)c(OCCCl)c(C#N)c1 ; CC(C)(c1ccc(OCc2cnc(NS(C)(=O)=O)nc2N2CCNCC2)cc1)c1cc(Cl)c(OCCCl)c(C#N)c1 ; CC(C)(c1ccc(OCC2CN(S(C)(=O)=O)C2)cc1)c1cc(Cl)c(OCCCl)c(C#N)c1 ; CC(C)(c1ccc(OCc2ccnc(NS(C)(=O)=O)n2)cc1)c1cnc(N2CC3(CCC3)C2)c(C#N)c1] 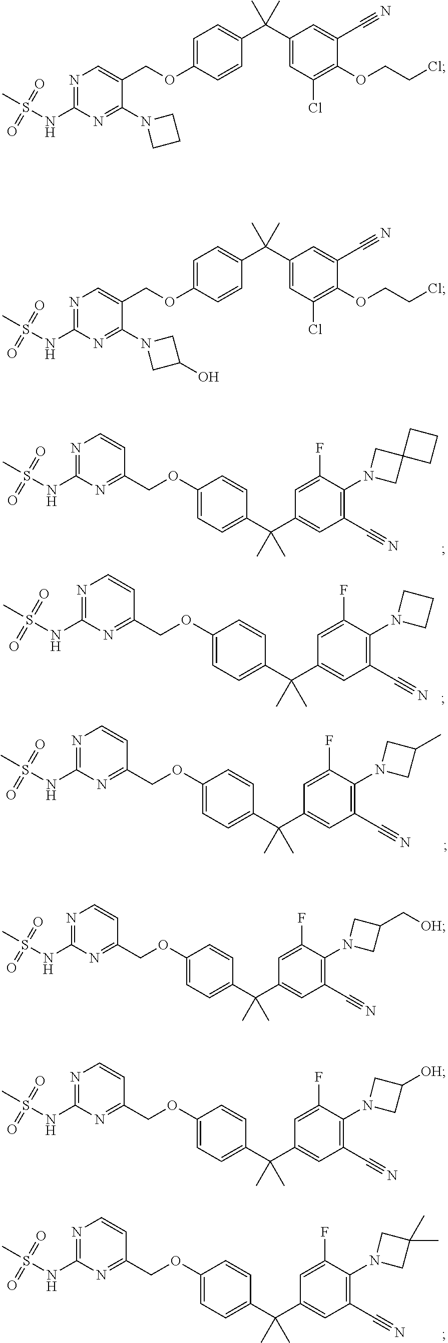 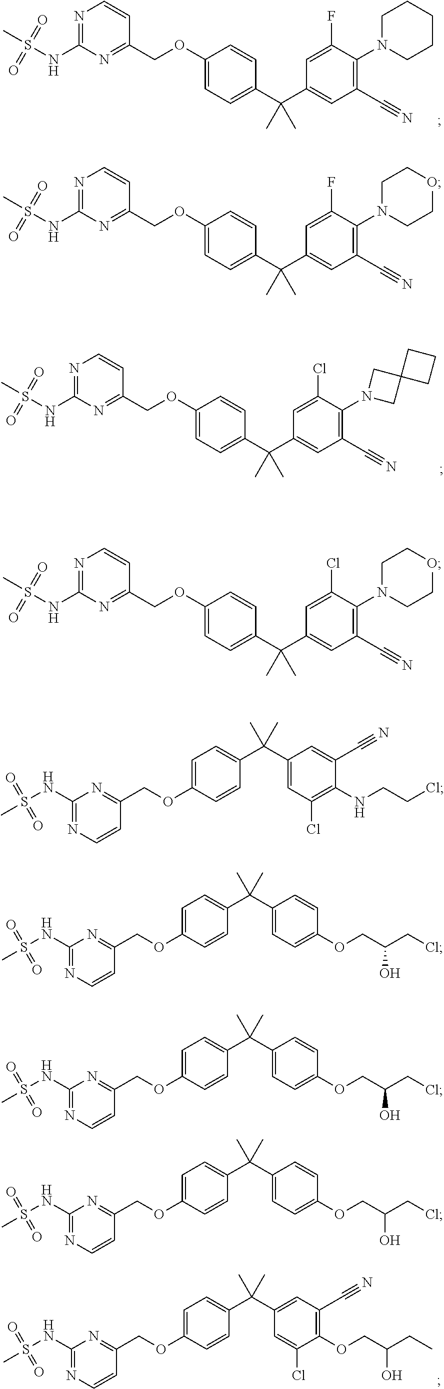 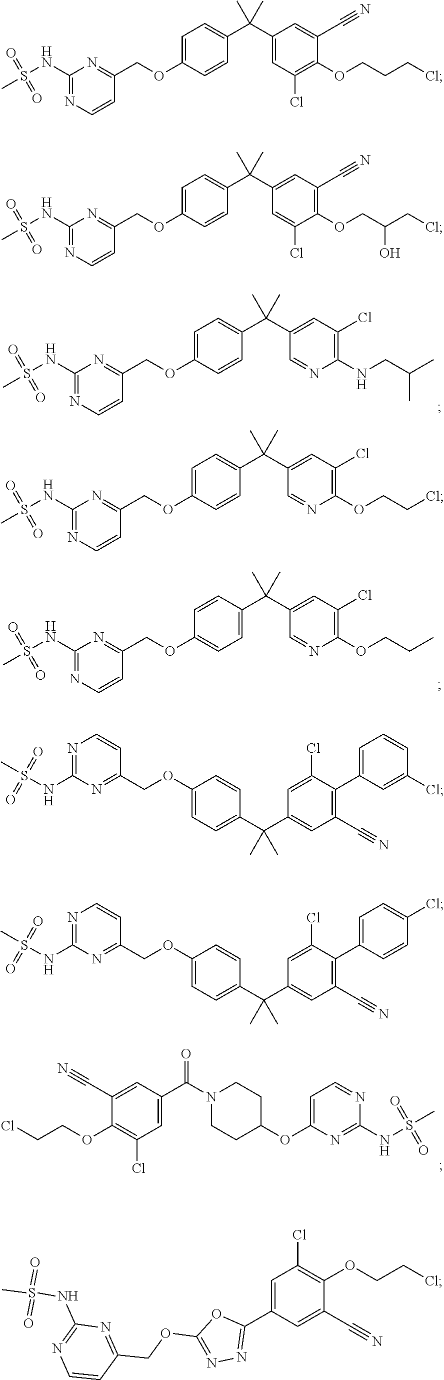 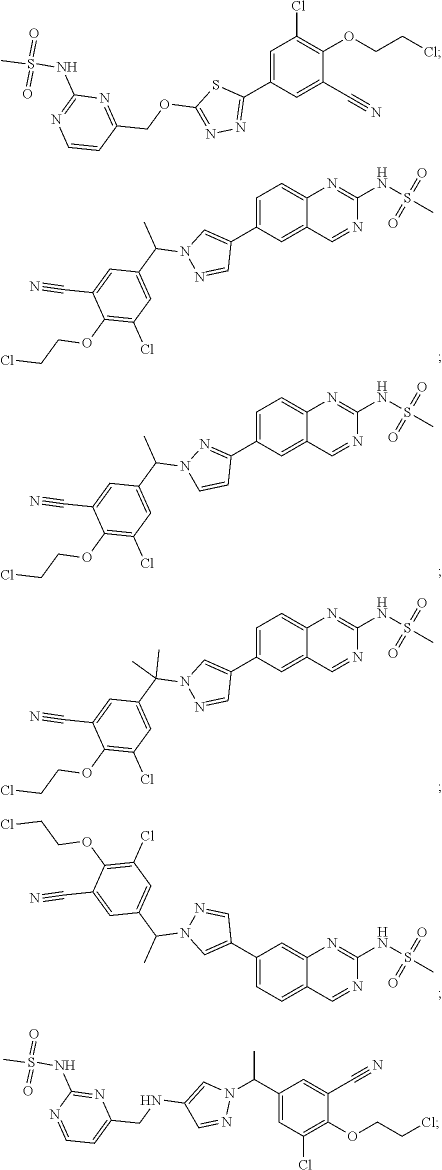 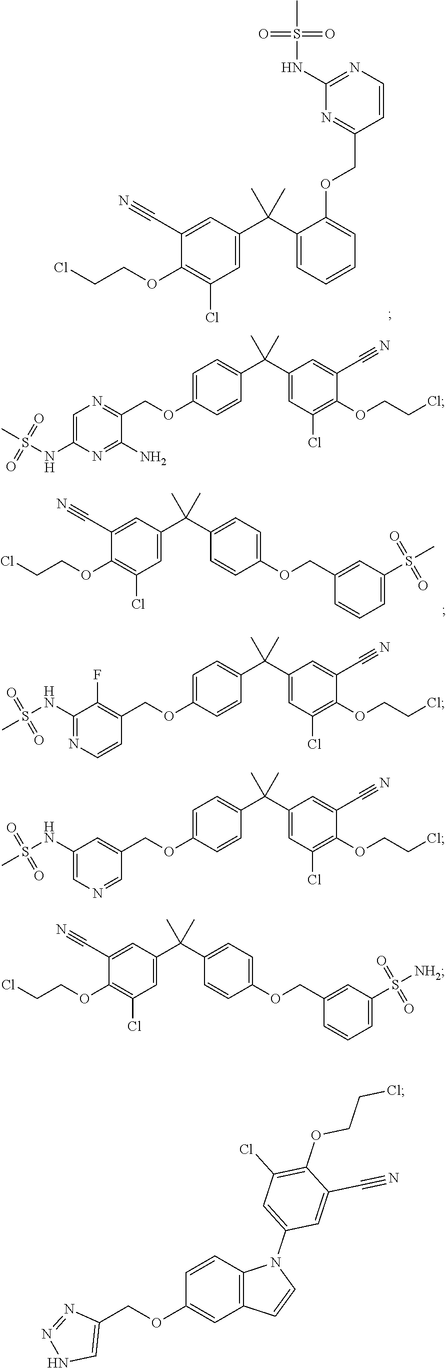 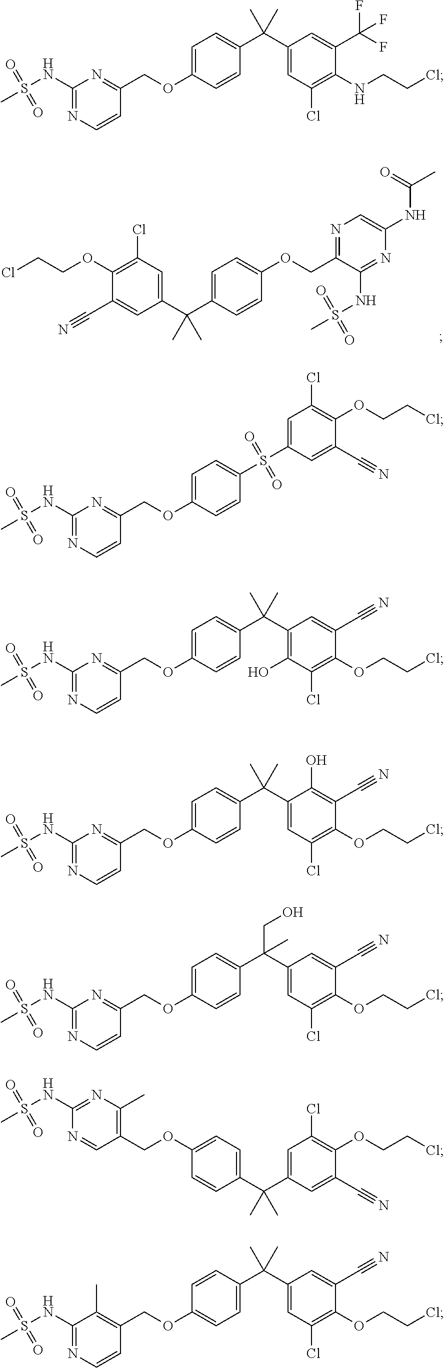 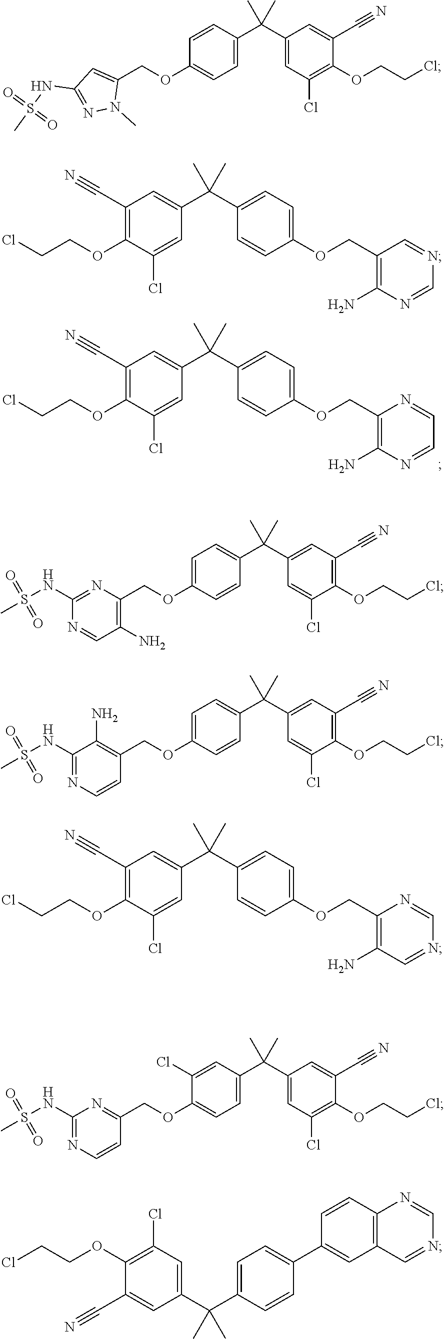 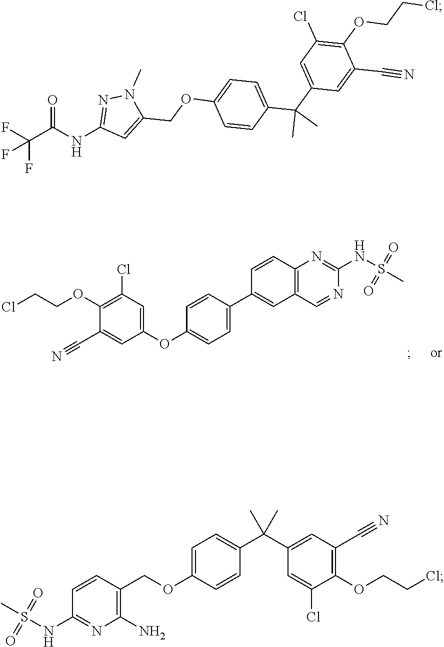
or a pharmaceutically acceptable salt thereof.

68.-72. (canceled)

73. A compound having the structure of formula (D): 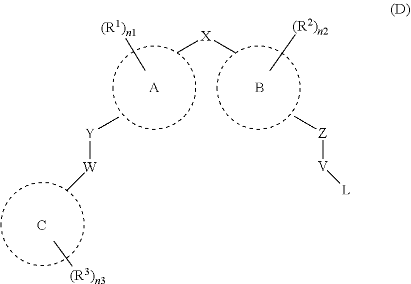
or a pharmaceutically acceptable salt, tautomer, stereoisomer or prodrug thereof, wherein:
A is absent;
B is 6- to 15-membered aryl ring;
C is a pyrimidine, triazine, or thiophene ring;
X is a bond, —CH₂—, —C(CH₃)₂—, or —C(═O)—;
Y is a bond or —O—;
W is —CH₂— or —C(CH₃)H—;
Z is a bond or —O—;
V is —CH₂— and L is halogen, —CHCl₂, —CCl₃, or —CF₃; or
V is —CH₂CH₂— and L is halogen;
R¹ and R² are each independently hydrogen, halogen, —CN, or —CF₃;
at least one R³ is —NHSO₂CH₃, —NHSO₂CH₂CH₃, —SO₂NH₂, or —SO₂CH₃; and the other R³ is, if present, —CN, —CF₃, C₁-C₃ alkyl, C₂-C₃ alkenyl, C₂-C₃ alkynyl, C₁-C₃ alkoxy, —S(C₁-C₃ alkyl), —SO₂(C₁-C₃ alkyl), —NH₂, —(C₁-C₃ alkyl)NH₂, —NHSO₂CH₃, —NHSO₂CF₃, —N(CH₃)SO₂CH₃, —NHSO₂CH₂CH₃, —N(CH₃)SO₂CH₂CH₃, —CH₂NHSO₂CH₃, —CH₂N(CH₃)SO₂CH₃, —SO₂NH₂, —CONH₂, —CON(C₁-C₃ alkyl)₂, —CONH(C₁-C₃ alkyl), —NHCO(C₁-C₃ alkyl), —NHCOO(C₁-C₃ alkyl), —N(CH₃)CO(C₁-C₃ alkyl), or —N(CH₃)COO(C₁-C₃ alkyl);
n1 and n2 are each independently 0, 1, or 2; and
n3 is 1, 2, 3, 4 or 5.

74. (canceled)

75. The compound of claim 73 having the structure: 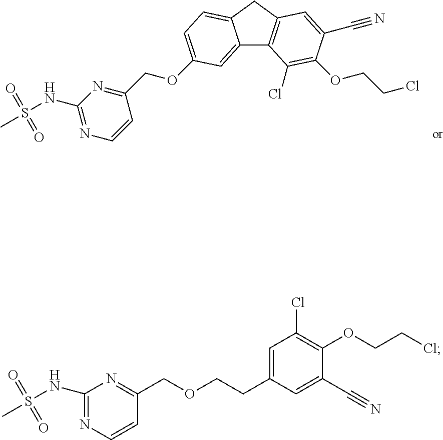 [SMILES: CS(=O)(=O)Nc1nccc(COc2ccc3c(c2)-c2c(cc(C#N)c(OCCCl)c2Cl)C3)n1 ; CS(=O)(=O)Nc1nccc(COCCc2cc(Cl)c(OCCCl)c(C#N)c2)n1]
or a pharmaceutically acceptable salt thereof.

76. (canceled)

77. A pharmaceutical composition comprising a compound of claim 1 and a pharmaceutically acceptable carrier.

78. A method for treating cancer, comprising administering the compound, pharmaceutically acceptable salt, tautomer, stereoisomer or prodrug of the compound of claim 1, to a subject in need thereof.

79. The method of claim 78, wherein the cancer is prostate cancer.

80. The method of claim 79, wherein:
a) the prostate cancer is metastatic castration-resistant prostate cancer or
b) the prostate cancer expresses full-length androgen receptor or truncated androgen receptor splice variant.

81. (canceled)
